(19) United States
(12) Patent Application Publication
(10) Pub. No.: US 2018/0166634 A1
(43) Pub. Date: Jun. 14, 2018

(54) ORGANIC LIGHT-EMITTING DEVICE AND COMPOUND

(71) Applicant: Samsung Electronics Co., Ltd., Suwon-si (KR)

(72) Inventors: Masaki NUMATA, Hwaseong-si (KR); Soonok JEON, Suwon-si (KR); Hiroshi MIYAZAKI, Hwaseong-si (KR); Saeyoun LEE, Suwon-si (KR); Myungsun SIM, Suwon-si (KR); Sooghang IHN, Hwaseong-si (KR); Hasup LEE, Seoul (KR); Yeonsook CHUNG, Seoul (KR); Youngchun KWON, Yongin-si (KR); Taerae KIM, Suwon-si (KR); Youngmin NAM, Seongnam-si (KR); Munbo SHIM, Yongin-si (KR); Won-joon SON, Yongin-si (KR)

(21) Appl. No.: 15/840,491

(22) Filed: Dec. 13, 2017

(30) Foreign Application Priority Data
Dec. 14, 2016 (KR) 10-2016-0170405

(51) Int. Cl. H01L 51/00; C07D 403/14; C09K 11/06; C07D 403/12

(52) U.S. Cl. CPC H01L 51/0067; C07D 403/14; H01L 51/5012; C09K 11/06; C07D 403/12; H01L 51/0072

(57) ABSTRACT

An organic light-emitting device including a first electrode, a second electrode facing the first electrode, and an organic layer disposed between the first electrode and the second electrode, wherein the organic layer includes an emission layer, and wherein the emission layer includes a fluorescent compound represented by a specific formula described in the specification.

### CROSS-REFERENCE TO RELATED APPLICATION

This application claims priority to Korean Patent Application No. 10-2016-0170405, filed on Dec. 14, 2016, in the Korean Intellectual Property Office, and all the benefits accruing therefrom under 35 U.S.C. § 119, the content of which is incorporated herein in its entirety by reference.

### BACKGROUND

### 1. Field

One or more embodiments relate to a compound and an organic light-emitting device including the compound.

### 2. Description of the Related Art

Organic light-emitting devices (OLEDs) are self-emission devices, which produce full-color images, and which have wide viewing angles, high contrast ratios, short response times, and excellent characteristics in terms of brightness, driving voltage, and response speed, compared to the devices in the art.

In an example, an organic light-emitting device includes an anode, a cathode, and an organic layer that is disposed between the anode and the cathode, wherein the organic layer includes an emission layer. A hole transport region may be disposed between the anode and the emission layer, and an electron transport region may be disposed between the emission layer and the cathode. Holes provided from the anode may move toward the emission layer through the hole transport region, and electrons provided from the cathode may move toward the emission layer through the electron transport region. Carriers, such as holes and electrons, recombine in an emission layer region to produce excitons. These excitons transit from an excited state to a ground state, thereby generating light.

Various types of organic light emitting devices are known. However, there still remains a need in OLEDs having low driving voltage, high efficiency, high brightness, and long lifespan.

### SUMMARY

One or more embodiments include an organic light-emitting device having high efficiency and a long lifespan, and a compound usable for manufacturing the organic light-emitting device having high efficiency and a long lifespan.

Additional aspects will be set forth in part in the description which follows and, in part, will be apparent from the description, or may be learned by practice of the presented embodiments.

According to one or more embodiments, an organic light-emitting device includes:

a first electrode,

a second electrode facing the first electrode, and

an organic layer disposed between the first electrode and the second electrode, the organic layer includes an emission layer,

wherein

the emission layer may include a fluorescent compound, of which a difference between a singlet excitation energy level and a triplet excitation energy level is greater than 0 electron volts and equal to or less than 0.5 electron volts,

a proportion of fluorescent emission components with respect to total emission components emitted from the emission layer may be about 90% or more, and the emission layer may not include a phosphorescent compound,

the fluorescent compound may include electron donor groups in the number of n1 and electron acceptor groups in the number of n2, wherein n1 and n2 may each independently be an integer from 1 to 10,

the electron donor groups in the number of n1 and the electron acceptor groups in the number of n2 may be chemically bonded to each other in random order, provided that a chemical bond between the electron donor group and the electron acceptor group is a carbon-carbon single bond,

at least one of the electron donor groups in the number of n1 may be an electron donor group represented by Formula 1A, and

the electron acceptor group may be selected from groups represented by Formula 1B:

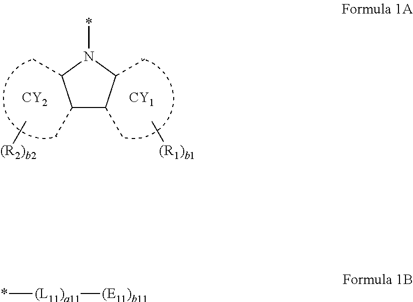

CY₁ and CY₂ in Formula 1A may each independently be selected from a benzene group, a naphthalene group, a carbazole group, a fluorene group, a dibenzofuran group, and a dibenzothiophene group,

R₁ and R₂ in Formula 1A may each independently be selected from:

hydrogen, deuterium, a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, and a π electron-depleted nitrogen-free C₂-C₆₀ heterocyclic group; and

a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, and a π electron-depleted nitrogen-free C₂-C₆₀ heterocyclic group, each substituted with at least one selected from deuterium, a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, a (C₁-C₁₀ alkyl)C₅-C₆₀ carbocyclic group, a di(C₁-C₁₀ alkyl)C₅-C₆₀ carbocyclic group, a (phenyl)C₅-C₆₀ carbocyclic group, a di(phenyl)C₅-C₆₀ carbocyclic group, a (biphenyl)C₅-C₆₀ carbocyclic group, a di(biphenyl)C₅-C₆₀ carbocyclic group, a π electron-depleted nitrogen-free C₂-C₆₀ heterocyclic group, a (C₁-C₁₀ alkyl) π electron-depleted nitrogen-free C₂-C₆₀ heterocyclic group, a di(C₁-C₁₀ alkyl) π electron-depleted nitrogen-free C₂-C₆₀ heterocyclic group, a (phenyl) π electron-depleted nitrogen-free C₂-C₆₀ heterocyclic group, a di(phenyl) π electron-depleted nitrogen-free C₂-C₆₀ heterocyclic group, a (biphenyl) π electron-depleted nitrogen-free C₂-C₆₀ heterocyclic group, and a di(biphenyl) π electron-depleted nitrogen-free C₂-C₆₀ heterocyclic group,

b1 and b2 in Formula 1A may each independently be an integer from 0 to 6,

“*” in Formula 1A indicates a binding site to a neighboring atom, provided that “*” in Formula 1A does not indicate a binding site to an electron acceptor group,

each of CY₁ and CY₂ in Formula 1A may optionally be additionally chemically bonded to at least one of an electron donor group and an electron acceptor group,

L₁₁ in Formula 1B may be selected from:

a single bond, a cyclopentane group, a cyclohexane group, a cycloheptane group, a cyclooctane group, a cyclopentene group, a cyclohexene group, a cycloheptene group, a benzene group, a naphthalene group, a fluorene group, a phenanthrene group, an anthracene group, a fluoranthene group, a triphenylene group, a pyrene group, a chrysene group, a pyrrole group, a thiophene group, a furan group, an imidazole group, a pyrazole group, a thiazole group, an isothiazole group, an oxazole group, an isoxazole group, a pyridine group, a pyrazine group, a pyrimidine group, a pyridazine group, an isoindole group, an indole group, an indazole group, a purine group, a quinoline group, an isoquinoline group, a benzoquinoline group, a quinoxaline group, a quinazoline group, a cinnoline group, a phenanthroline group, a benzimidazole group, a benzofuran group, a benzothiophene group, a benzoxazole group, an isobenzoxazole group, a triazole group, a tetrazole group, an oxadiazole group, a triazine group, a dibenzofuran group, a dibenzothiophene group, a benzocarbazole group, a dibenzocarbazole group, an imidazopyridine group, an imidazopyrimidine group, an azaindole group, an azaindene group, an azabenzofuran group, an azabenzothiophene group, an azacarbazole group, an azafluorene group, an azadibenzofuran group, and an azadibenzothiophene group; and

a cyclopentane group, a cyclohexane group, a cycloheptane group, a cyclooctane group, a cyclopentene group, a cyclohexene group, a cycloheptene group, a benzene group, a naphthalene group, a fluorene group, a phenanthrene group, an anthracene group, a fluoranthene group, a triphenylene group, a pyrene group, a chrysene group, a pyrrole group, a thiophene group, a furan group, an imidazole group, a pyrazole group, a thiazole group, an isothiazole group, an oxazole group, an isoxazole group, a pyridine group, a pyrazine group, a pyrimidine group, a pyridazine group, an isoindole group, an indole group, an indazole group, a purine group, a quinoline group, an isoquinoline group, a benzoquinoline group, a quinoxaline group, a quinazoline group, a cinnoline group, a phenanthroline group, a benzimidazole group, a benzofuran group, a benzothiophene group, a benzoxazole group, an isobenzoxazole group, a triazole group, a tetrazole group, an oxadiazole group, a triazine group, a dibenzofuran group, a dibenzothiophene group, a benzocarbazole group, a dibenzocarbazole group, an imidazopyridine group, an imidazopyrimidine group, an azaindole group, an azaindene group, an azabenzofuran group, an azabenzothiophene group, an azacarbazole group, an azafluorene group, an azadibenzofuran group, and an azadibenzothiophene group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a (C₁-C₂₀ alkyl)phenyl group, a di(C₁-C₂₀ alkyl)phenyl group, a (C₆-C₂₀ aryl)phenyl group, a di(C₆-C₂₀ aryl)phenyl group, a (C₃-C₂₀ heteroaryl)phenyl group, a di(C₃-C₂₀ heteroaryl)phenyl group, a pyridinyl group, a (C₁-C₂₀ alkyl)pyridinyl group, a di(C₁-C₂₀ alkyl)pyridinyl group, a (C₆-C₂₀ aryl)pyridinyl group, a di(C₆-C₂₀ aryl)pyridinyl group, a (C₃-C₂₀ heteroaryl)pyridinyl group, a di(C₃-C₂₀ heteroaryl)pyridinyl group, a pyrimidinyl group, a (C₁-C₂₀ alkyl)pyrimidinyl group, a di(C₁-C₂₀ alkyl)pyrimidinyl group, a (C₆-C₂₀ aryl)pyrimidinyl group, a di(C₆-C₂₀ aryl)pyrimidinyl group, a (C₃-C₂₀ heteroaryl)pyrimidinyl group, a di(C₃-C₂₀ heteroaryl)pyrimidinyl group, a triazinyl group, a (C₁-C₂₀ alkyl)triazinyl group, a di(C₁-C₂₀ alkyl)triazinyl group, a (C₆-C₂₀ aryl)triazinyl group, a di(C₆-C₂₀ aryl)triazinyl group, a (C₃-C₂₀ heteroaryl)triazinyl group, and a di(C₃-C₂₀ heteroaryl)triazinyl group,

a11 in Formula 1B may be an integer from 1 to 3,

E₁₁ in Formula 1B may be selected from:

—F, —CFH₂, —CF₂H, —CF₃, and —CN;

a C₁-C₆₀ alkyl group or a C₁-C₆₀ alkoxy group, substituted with at least one selected from —F, —CFH₂, —CF₂H, —CF₃, and —CN; and

a substituted or unsubstituted π electron-depleted nitrogen-containing C₂-C₆₀ heterocyclic group,

b11 in Formula 1B may be an integer from 1 to 5,

a bond between L₁₁ and E₁₁ in Formula 1B may be a carbon-carbon single bond or a carbon-fluorine single bond,

“*” in Formula 1B indicates a binding site to a neighboring carbon, and

at least one substituent of the substituted π electron-depleted nitrogen-containing C₂-C₆₀ heterocyclic group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and

—N(Q₃₁)(Q₃₂), —Si(Q₃₃)(Q₃₄)(Q₃₅), —B(Q₃₆)(Q₃₇), and —P(═O)(Q₃₈)(Q₃₉); and

Q₁₁ to Q₁₉, Q₂₁ to Q₂₉, and Q₃₁ to Q₃₉ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one of a C₁-C₆₀ alkyl group and a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group.

According to one or more embodiments, a compound represented by one of Formulae 9-1 to 9-9 is provided:

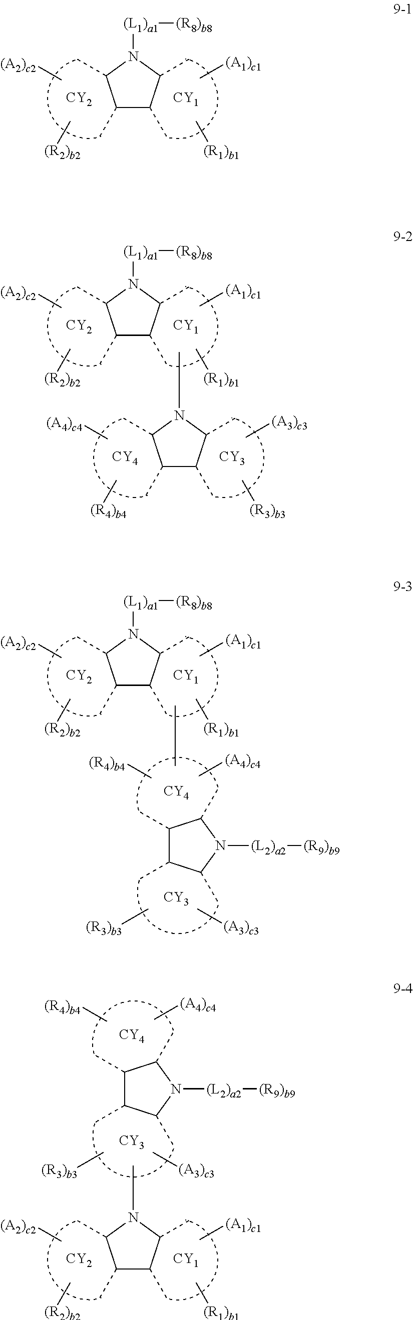

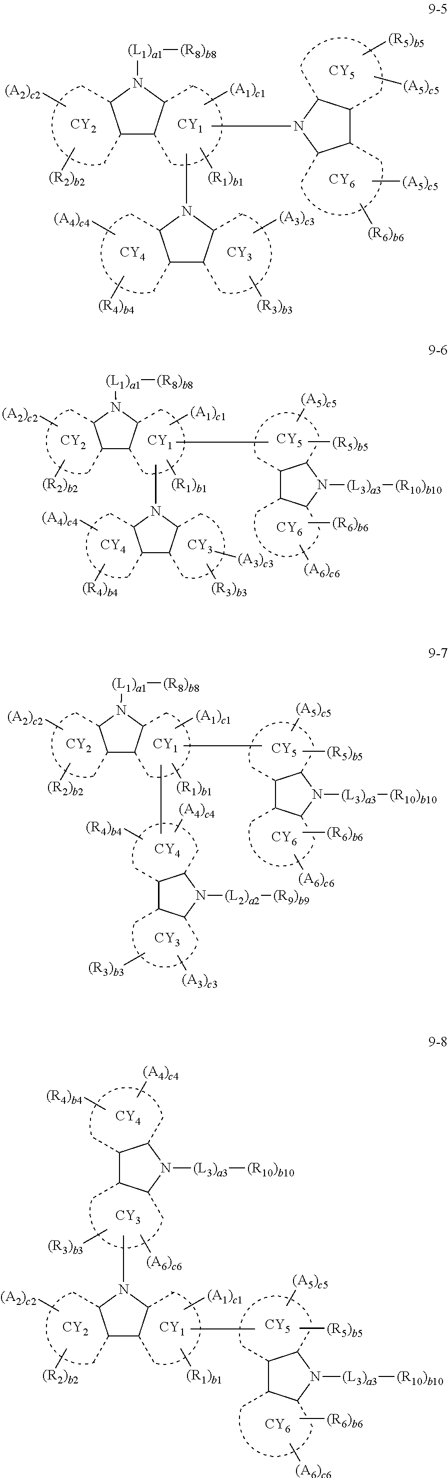

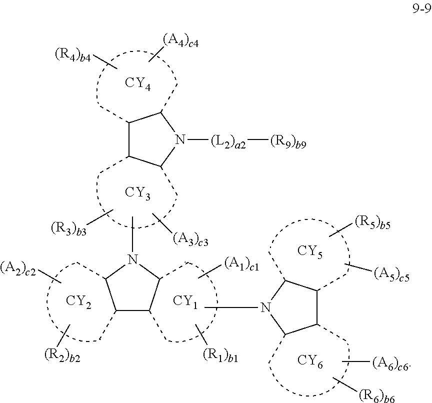

The groups and variables in Formulae 9-1 to 9-9 are the same as described below.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic view of an organic light-emitting device according to an embodiment;

FIG. 2 is a graph of UV absorbance (arbitrary units, a. u.) versus wavelength (nanometers, nm), showing an ultraviolet (UV) absorption spectrum of Compound FD(5) and a photoluminescence (PL) spectrum of Compound 1;

FIG. 3 is a graph of UV absorbance (arbitrary units, a. u.) versus wavelength (nanometers, nm), showing a UV absorption spectrum of Compound FD(5) and a PL spectrum of Compound 2;

FIG. 4 is a graph of UV absorbance (arbitrary units, a. u.) versus wavelength (nanometers, nm), showing a UV absorption spectrum of Compound FD(5) and a PL spectrum of Compound 3; and

FIG. 5 is a graph of UV absorbance (arbitrary units, a. u.) versus wavelength (nanometers, nm), showing a UV absorption spectrum of Compound FD(5) and a PL spectrum of Compound 4.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

In an embodiment, an organic light-emitting device is provided. The organic light-emitting device according to an embodiment may include a first electrode, a second electrode facing the first electrode, and an organic layer between the first electrode and the second electrode, the organic layer including an emission layer.

The emission layer may include a fluorescent compound, of which a difference between a singlet excitation energy level and a triplet excitation energy level is greater than 0 electron volts (eV) and equal to or less than 0.5 eV, for example, greater than 0.01 eV and equal to or less than 0.3 eV. While not wishing to be bound by theory, when the difference between the singlet excitation energy level and the triplet excitation energy level of the fluorescent compound is within the above-described range, a delayed fluorescence caused by reverse intersystem crossing of the fluorescent compound may be effectively achieved. That is, the fluorescent compound may be a thermally activated delayed fluorescent (TADF) compound.

A proportion of fluorescent emission components with respect to total emission components emitted from the emission layer may be about 90% or more, for example, about 95% or more (for example, about 98% or more), and the emission layer may not include a phosphorescent emission compound capable of emitting phosphorescence light (for example, an organometallic compound including a heavy metal). Therefore, the emission layer is quite different from a phosphorescent emission layer which includes a phosphorescent dopant, and in which a proportion of phosphorescent emission components with respect to total emission components is, for example, about 80% or more.

The fluorescent compound may include electron donor groups in the number of n1 and electron acceptor groups in the number of n2, wherein n1 and n2 may each independently be an integer from 1 to 10. For example, n1 and n2 may each independently be 1, 2, or 3, but embodiments of the present disclosure are not limited thereto.

The electron donor groups in the number of n1 and the electron acceptor groups in the number of n2 may be chemically bonded to each other in random order, provided that at least one of chemical bonds between the electron donor groups and the electron acceptor groups, for example, all chemical bonds between the electron donor groups and the electron acceptor groups, may be a carbon-carbon single bond. Due to this bonding, the fluorescent compound may have excellent decomposition resistance. Thus, it is possible to prevent the electron donor groups and the electron acceptor groups of the fluorescent compound from being separated from each other during the storing and/or driving of the organic light-emitting device including the fluorescent compound. Consequently, it is possible to prevent a reduction in the efficiency and/or lifespan of the organic light-emitting device. Therefore, the organic light-emitting device including the fluorescent compound may have high efficiency and a long lifespan while emitting “fluorescence”.

At least one of the electron donor groups in the number of n1 may be an electron donor group represented by Formula 1A, and the electron acceptor group may be selected from groups represented by Formula 1B:

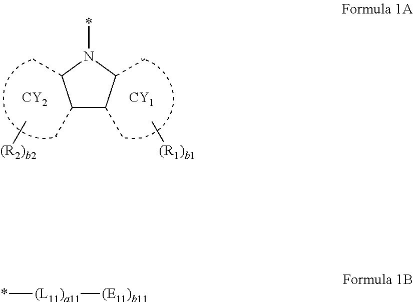

CY₁ and CY₂ in Formula 1A may each independently be selected from a benzene group, a naphthalene group, a carbazole group, a fluorene group, a dibenzofuran group, and a dibenzothiophene group.

For example, at least one of CY₁ and CY₂ in Formula 1A may be a benzene group, but embodiments of the present disclosure are not limited thereto.

R₁ and R₂ in Formula 1A may each independently be selected from:

hydrogen, deuterium, a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, and a π electron-depleted nitrogen-free C₂-C₆₀ heterocyclic group; and

a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, and a π electron-depleted nitrogen-free C₂-C₆₀ heterocyclic group, each substituted with at least one selected from deuterium, a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, a (C₁-C₁₀ alkyl)C₅-C₆₀ carbocyclic group, a di(C₁-C₁₀ alkyl)C₅-C₆₀ carbocyclic group, a (phenyl)C₅-C₆₀ carbocyclic group, a di(phenyl)C₅-C₆₀ carbocyclic group, a (biphenyl)C₅-C₆₀ carbocyclic group, a di(biphenyl)C₅-C₆₀ carbocyclic group, a π electron-depleted nitrogen-free C₂-C₆₀ heterocyclic group, a (C₁-C₁₀ alkyl) π electron-depleted nitrogen-free C₂-C₆₀ heterocyclic group, a di(C₁-C₁₀ alkyl) π electron-depleted nitrogen-free C₂-C₆₀ heterocyclic group, a (phenyl) π electron-depleted nitrogen-free C₂-C₆₀ heterocyclic group, a di(phenyl) π electron-depleted nitrogen-free C₂-C₆₀ heterocyclic group, a (biphenyl) π electron-depleted nitrogen-free C₂-C₆₀ heterocyclic group, and a di(biphenyl) π electron-depleted nitrogen-free C₂-C₆₀ heterocyclic group.

The term “π electron-depleted nitrogen-free C₂-C₆₀ heterocyclic group” as used herein means a C₂-C₆₀ heterocyclic group that includes carbon atoms and at least one heteroatom selected from N, O, S, Si, and P as a ring-forming atom, but does not include “π electron-depleted nitrogen”. Also, the term “π electron-depleted nitrogen-containing C₂-C₆₀ heterocyclic group” as used herein means a C₂-C₆₀ heterocyclic group that essentially includes at least one “π electron-depleted nitrogen” as a ring-forming atom.

For example, a “carbazole group” belongs to the “π electron-depleted nitrogen-free C₂-C₆₀ heterocyclic group”, and a “triazine group” belongs to a “π electron-depleted nitrogen-containing C₂-C₆₀ heterocyclic group”.

The term “(C₁-C₁₀ alkyl)C₅-C₆₀ carbocyclic group” as used herein means a C₅-C₆₀ carbocyclic group substituted with one C₁-C₁₀ alkyl group, and the term “di(C₁-C₁₀ alkyl)C₅-C₆₀ carbocyclic group” as used herein means a C₅-C₆₀ carbocyclic group substituted with two C₁-C₁₀ alkyl groups. Similarly, other terms may also be understood.

In one or more embodiments, R₁ and R₂ in Formula 1A may each independently be selected from:

hydrogen, deuterium, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a furanyl group, a thiophenyl group, an indolyl group, a benzofuranyl group, a benzothiophenyl group, a carbazolyl group, a dibenzofuranyl group, a dibenzothiophenyl group, a benzocarbazolyl group, a naphtho benzofuranyl group, a naphtho benzothiophenyl group, a dibenzocarbazolyl group, a dinaphthofuranyl group, a dinaphthothiophenyl group, an indolofluorenyl group, an indolocarbazolyl group, an indolodibenzofuranyl group, and an indolodibenzothiophenyl group; and

a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a furanyl group, a thiophenyl group, an indolyl group, a benzofuranyl group, a benzothiophenyl group, a carbazolyl group, a dibenzofuranyl group, a dibenzothiophenyl group, a benzocarbazolyl group, a naphtho benzofuranyl group, a naphtho benzothiophenyl group, a dibenzocarbazolyl group, a dinaphthofuranyl group, a dinaphthothiophenyl group, an indolofluorenyl group, an indolocarbazolyl group, an indolodibenzofuranyl group, and an indolodibenzothiophenyl group, each substituted with at least one selected from deuterium, a C₁-C₂₀ alkyl group, a C₁-C₂₀ alkoxy group, a phenyl group, a biphenyl group, a terphenyl group, a naphthyl group, a fluorenyl group, a dimethylfluorenyl group, a diphenylfluorenyl group, a carbazolyl group, a phenylcarbazolyl group, a biphenylcarbazolyl group, a dibenzofuranyl group, and a dibenzothiophenyl group,

but embodiments of the present disclosure are not limited thereto.

In one or more embodiments, R₁ and R₂ in Formula 1A may each independently be selected from hydrogen, deuterium, a C₁-C₂₀ alkyl group, a C₁-C₂₀ alkoxy group, and groups represented by Formulae 2-1 to 2-26, but embodiments of the present disclosure are not limited thereto:

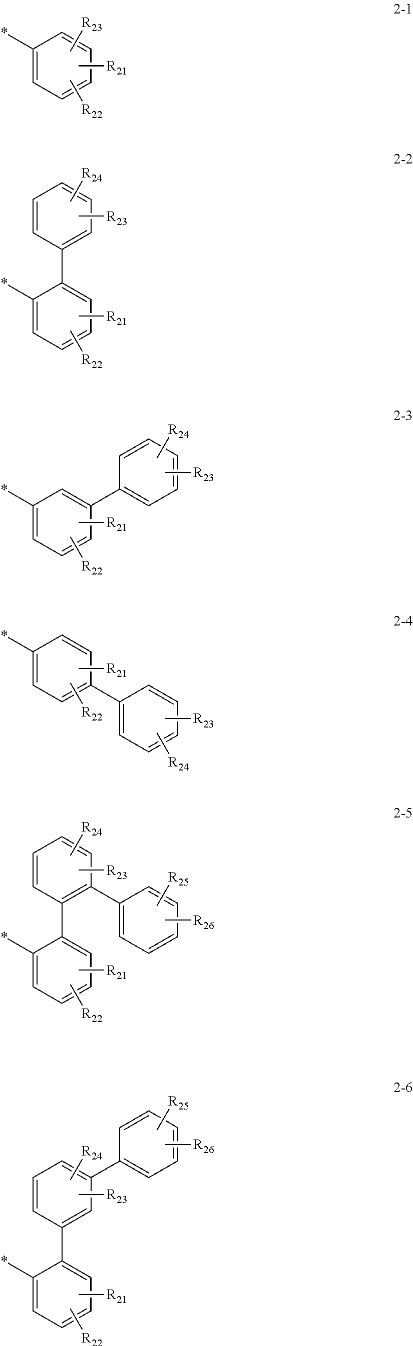

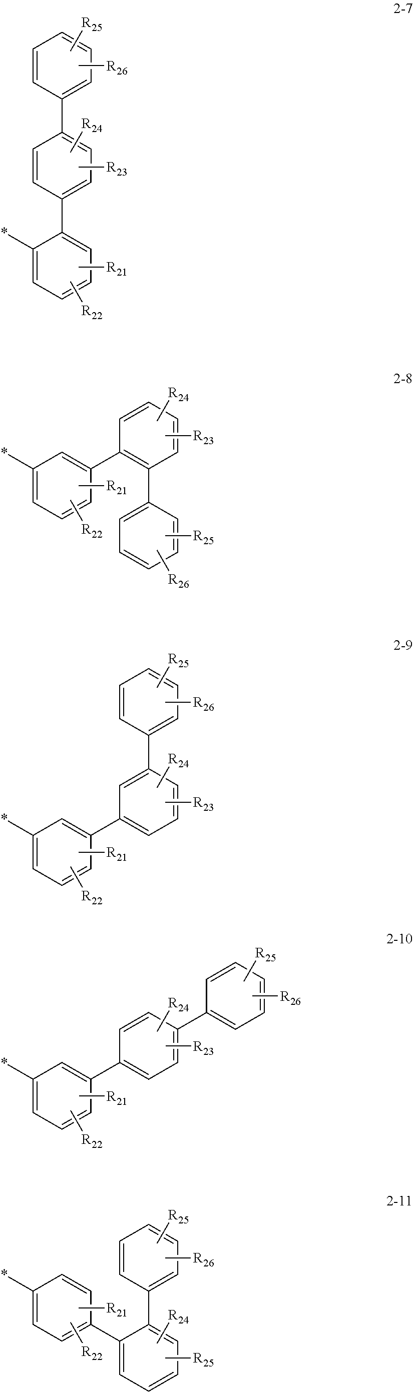

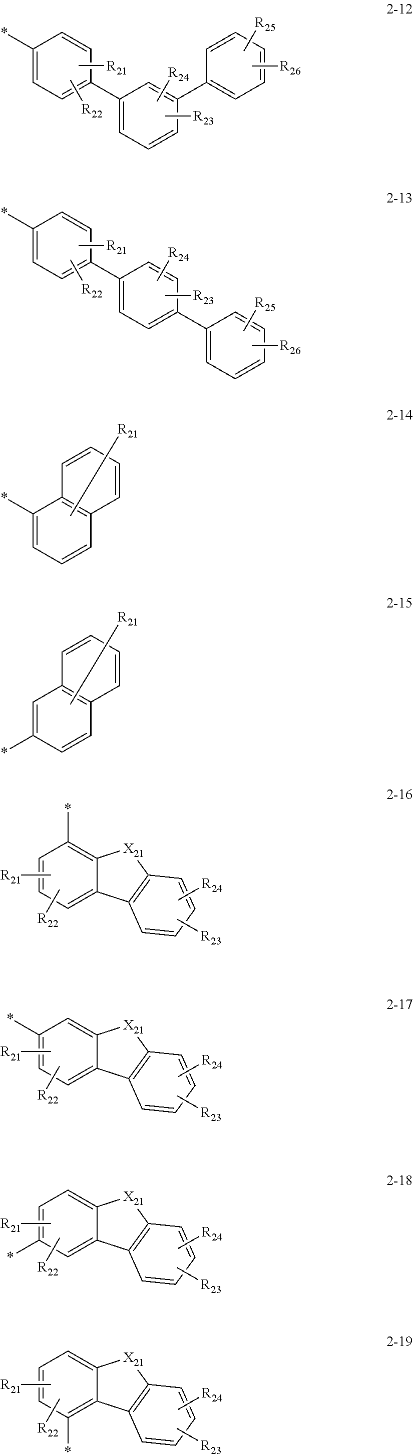

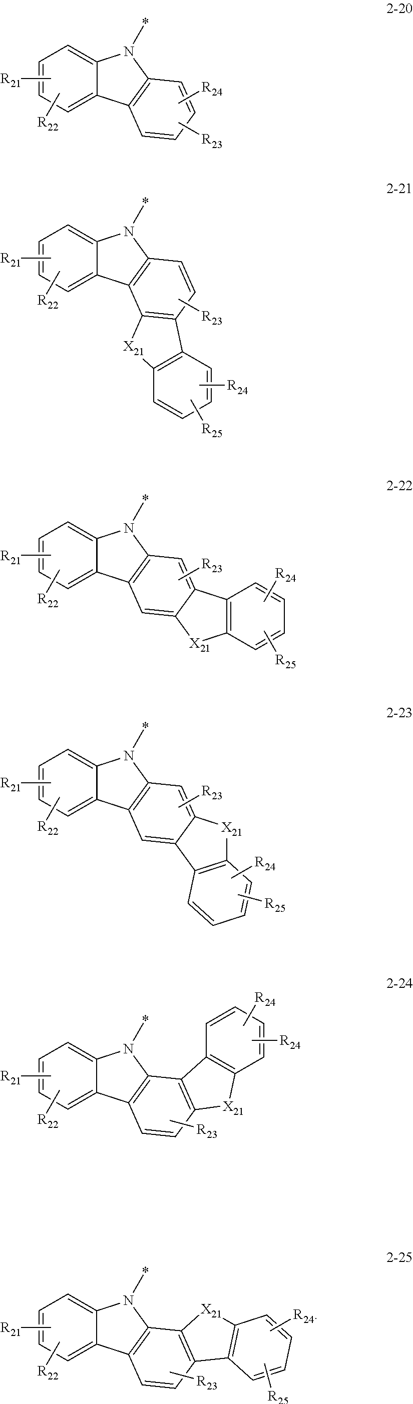

In Formulae 2-1 to 2-26,

X₂₁ may be C(R₂₇)(R₂₈), N(R₂₉), O, or S,

R₂₁ to R₂₉ may each independently be selected from hydrogen, deuterium, a C₁-C₂₀ alkyl group, a C₁-C₂₀ alkoxy group, a phenyl group, a biphenyl group, a terphenyl group, a naphthyl group, a fluorenyl group, a dimethylfluorenyl group, a diphenylfluorenyl group, a carbazolyl group, a phenylcarbazolyl group, a biphenylcarbazolyl group, a dibenzofuranyl group, and a dibenzothiophenyl group, and

“*” indicates a binding site to a neighboring atom.

b1 and b2 in Formula 1A respectively indicate the number of groups R₁ and the number of groups R₂ and may each independently be an integer from 0 to 6, wherein, when b1 is two or more, two or more groups R₁ may be identical to or different from each other, and when b2 is two or more, two or more groups R₂ may be identical to or different from each other.

In one or more embodiments, b1 and b2 in Formula 1A may each independently be 0, 1, or 2, and the sum of b1 and b2 may be 0, 1, or 2.

In one or more embodiments, R₁ and R₂ in Formula 1A may each independently be selected from hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, a di(phenyl)phenyl group, a fluorenyl group, a (C₁-C₁₀ alkyl)fluorenyl group, a di(C₁-C₁₀ alkyl)fluorenyl group, a (phenyl)fluorenyl group, a di(phenyl)fluorenyl group, a carbazolyl group, a (C₁-C₁₀ alkyl)carbazolyl group, a di(C₁-C₁₀ alkyl)carbazolyl group, a (phenyl)carbazolyl group, a di(phenyl)carbazolyl group, a dibenzofuranyl group, a (C₁-C₁₀ alkyl)dibenzofuranyl group, a di(C₁-C₁₀ alkyl)dibenzofuranyl group, a (phenyl)dibenzofuranyl group, a di(phenyl)dibenzofuranyl group, a dibenzothiophenyl group, a (C₁-C₁₀ alkyl)dibenzothiophenyl group, a di(C₁-C₁₀ alkyl)dibenzothiophenyl group, a (phenyl)dibenzothiophenyl group, a di(phenyl)dibenzothiophenyl group, an indolofluorenyl group, a (C₁-C₁₀ alkyl)indolofluorenyl group, a di(C₁-C₁₀ alkyl)indolofluorenyl group, a (phenyl)indolofluorenyl group, a di(phenyl)indolofluorenyl group, an indolocarbazole group, a (C₁-C₁₀ alkyl)indolocarbazole group, a di(C₁-C₁₀ alkyl)indolocarbazole group, a (phenyl)indolocarbazole group, a di(phenyl)indolocarbazole group, an indolodibenzofuranyl group, a (C₁-C₁₀ alkyl)indolodibenzofuranyl group, a di(C₁-C₁₀ alkyl)indolodibenzofuranyl group, a (phenyl)indolodibenzofuranyl group, a di(phenyl)indolodibenzofuranyl group, an indolodibenzothiophenyl group, a (C₁-C₁₀ alkyl)indolodibenzothiophenyl group, a di(C₁-C₁₀ alkyl)indolodibenzothiophenyl group, a (phenyl)indolodibenzothiophenyl group, and a di(phenyl)indolodibenzothiophenyl group, and b1 and b2 may each independently be 0, 1, or 2, but embodiments of the present disclosure are not limited thereto.

“*” in Formula 1A indicates a binding site to a neighboring atom, provided that “*” in Formula 1A does not indicate a binding site to an electron acceptor group represented by Formula 1B. For example, “*” in Formula 1A indicates a binding site to a group represented by *-(L₁)ₐ₁-(R₈)ₐ₈, a binding site to a group represented by *-(L₂)ₐ₂-(R₉)ₐ₉, a binding site to a group represented by *-(L₃)ₐ₃-(R₁₀)ₐ₁₀, or a binding site to a neighboring electron donor group in Formulae 9-1 to 9-9.

Each of CY₁ and CY₂ in Formula 1A may optionally be additionally chemically bonded to at least one of an electron donor group and an electron acceptor group.

The electron donor group represented by Formula 1A may be derived from any combination of CY₁, CY₂, R₁, R₂, a1, and a2 described herein.

L₁₁ in Formula 1B may be selected from:

a single bond, a cyclopentane group, a cyclohexane group, a cycloheptane group, a cyclooctane group, a cyclopentene group, a cyclohexene group, a cycloheptene group, a benzene group, a naphthalene group, a fluorene group, a phenanthrene group, an anthracene group, a fluoranthene group, a triphenylene group, a pyrene group, a chrysene group, a pyrrole group, a thiophene group, a furan group, an imidazole group, a pyrazole group, a thiazole group, an isothiazole group, an oxazole group, an isoxazole group, a pyridine group, a pyrazine group, a pyrimidine group, a pyridazine group, an isoindole group, an indole group, an indazole group, a purine group, a quinoline group, an isoquinoline group, a benzoquinoline group, a quinoxaline group, a quinazoline group, a cinnoline group, a phenanthroline group, a benzimidazole group, a benzofuran group, a benzothiophene group, a benzoxazole group, an isobenzoxazole group, a triazole group, a tetrazole group, an oxadiazole group, a triazine group, a dibenzofuran group, a dibenzothiophene group, a benzocarbazole group, a dibenzocarbazole group, an imidazopyridine group, an imidazopyrimidine group, an azaindole group, an azaindene group, an azabenzofuran group, an azabenzothiophene group, an azacarbazole group, an azafluorene group, an azadibenzofuran group, and an azadibenzothiophene group; and

a cyclopentane group, a cyclohexane group, a cycloheptane group, a cyclooctane group, a cyclopentene group, a cyclohexene group, a cycloheptene group, a benzene group, a naphthalene group, a fluorene group, a phenanthrene group, an anthracene group, a fluoranthene group, a triphenylene group, a pyrene group, a chrysene group, a pyrrole group, a thiophene group, a furan group, an imidazole group, a pyrazole group, a thiazole group, an isothiazole group, an oxazole group, an isoxazole group, a pyridine group, a pyrazine group, a pyrimidine group, a pyridazine group, an isoindole group, an indole group, an indazole group, a purine group, a quinoline group, an isoquinoline group, a benzoquinoline group, a quinoxaline group, a quinazoline group, a cinnoline group, a phenanthroline group, a benzimidazole group, a benzofuran group, a benzothiophene group, a benzoxazole group, an isobenzoxazole group, a triazole group, a tetrazole group, an oxadiazole group, a triazine group, a dibenzofuran group, a dibenzothiophene group, a benzocarbazole group, a dibenzocarbazole group, an imidazopyridine group, an imidazopyrimidine group, an azaindole group, an azaindene group, an azabenzofuran group, an azabenzothiophene group, an azacarbazole group, an azafluorene group, an azadibenzofuran group, and an azadibenzothiophene group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a (C₁-C₂₀ alkyl)phenyl group, a di(C₁-C₂₀ alkyl)phenyl group, a (C₆-C₂₀ aryl)phenyl group, a di(C₆-C₂₀ aryl)phenyl group, a (C₃-C₂₀ heteroaryl)phenyl group, a di(C₃-C₂₀ heteroaryl)phenyl group, a pyridinyl group, a (C₁-C₂₀ alkyl)pyridinyl group, a di(C₁-C₂₀ alkyl)pyridinyl group, a (C₆-C₂₀ aryl)pyridinyl group, a di(C₆-C₂₀ aryl)pyridinyl group, a (C₃-C₂₀ heteroaryl)pyridinyl group, a di(C₃-C₂₀ heteroaryl)pyridinyl group, a pyrimidinyl group, a (C₁-C₂₀ alkyl)pyrimidinyl group, a di(C₁-C₂₀ alkyl)pyrimidinyl group, a (C₆-C₂₀ aryl)pyrimidinyl group, a di(C₆-C₂₀ aryl)pyrimidinyl group, a (C₃-C₂₀ heteroaryl)pyrimidinyl group, a di(C₃-C₂₀ heteroaryl)pyrimidinyl group, a triazinyl group, a (C₁-C₂₀ alkyl)triazinyl group, a di(C₁-C₂₀ alkyl)triazinyl group, a (C₆-C₂₀ aryl)triazinyl group, a di(C₆-C₂₀ aryl)triazinyl group, a (C₃-C₂₀ heteroaryl)triazinyl group, and a di(C₃-C₂₀ heteroaryl)triazinyl group.

L₁₁ in Formula 1B does not include a “carbazole ring”.

In one or more embodiments, L₁₁ in Formula 1B may be selected from:

a single bond, a benzene group, a naphthalene group, a fluorene group, a pyridine group, a pyrazine group, a pyrimidine group, a pyridazine group, a quinoline group, an isoquinoline group, a benzoquinoline group, a quinoxaline group, a quinazoline group, and a triazine group; and

a benzene group, a naphthalene group, a fluorene group, a pyridine group, a pyrazine group, a pyrimidine group, a pyridazine group, a quinoline group, an isoquinoline group, a benzoquinoline group, a quinoxaline group, a quinazoline group, and a triazine group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a (C₁-C₂₀ alkyl)phenyl group, a di(C₁-C₂₀ alkyl)phenyl group, a (C₆-C₂₀ aryl)phenyl group, a di(C₆-C₂₀ aryl)phenyl group, a (C₃-C₂₀ heteroaryl)phenyl group, a di(C₃-C₂₀ heteroaryl)phenyl group, a pyridinyl group, a (C₁-C₂₀ alkyl)pyridinyl group, a di(C₁-C₂₀ alkyl)pyridinyl group, a (C₆-C₂₀ aryl)pyridinyl group, a di(C₆-C₂₀ aryl)pyridinyl group, a (C₃-C₂₀ heteroaryl)pyridinyl group, a di(C₃-C₂₀ heteroaryl)pyridinyl group, a pyrimidinyl group, a (C₁-C₂₀ alkyl)pyrimidinyl group, a di(C₁-C₂₀ alkyl)pyrimidinyl group, a (C₆-C₂₀ aryl)pyrimidinyl group, a di(C₆-C₂₀ aryl)pyrimidinyl group, a (C₃-C₂₀ heteroaryl)pyrimidinyl group, a di(C₃-C₂₀ heteroaryl)pyrimidinyl group, a triazinyl group, a (C₁-C₂₀ alkyl)triazinyl group, a di(C₁-C₂₀ alkyl)triazinyl group, a (C₆-C₂₀ aryl)triazinyl group, a di(C₆-C₂₀ aryl)triazinyl group, a (C₃-C₂₀ heteroaryl)triazinyl group, and a di(C₃-C₂₀ heteroaryl)triazinyl group,

but embodiments of the present disclosure are not limited thereto.

a11 in Formula 1B indicates the number of groups L₁₁ and may be an integer from 1 to 3, wherein, when a11 is two or more, two or more groups L₁₁ may be identical to or different from each other.

In one or more embodiments, a11 may be 1 or 2, but embodiments of the present disclosure are not limited thereto.

E₁₁ in Formula 1B may be selected from:

—F, —CFH₂, —CF₂H, —CF₃, and —CN;

a C₁-C₆₀ alkyl group or a C₁-C₆₀ alkoxy group, substituted with at least one selected from —F, —CFH₂, —CF₂H, —CF₃, and —CN; and

a substituted or unsubstituted π electron-depleted nitrogen-containing C₂-C₆₀ heterocyclic group.

For example, E₁₁ in Formula 1B may be selected from:

—F, —CFH₂, —CF₂H, —CF₃, and —CN;

a C₁-C₂₀ alkyl group substituted with at least one selected from —F, —CFH₂, —CF₂H, —CF₃, and —CN; and

groups represented by Formulae 3-1 to 3-14:

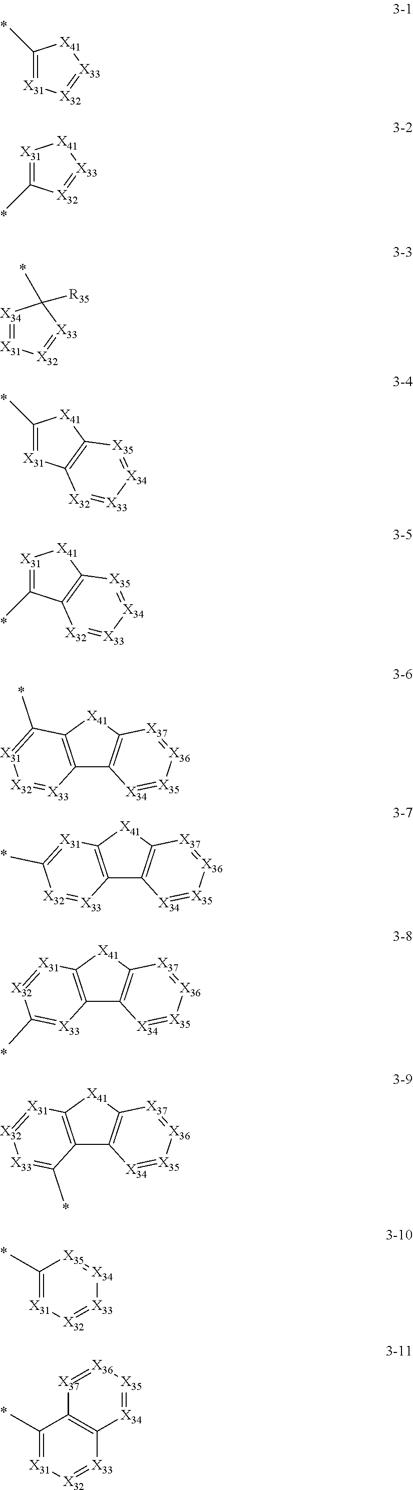

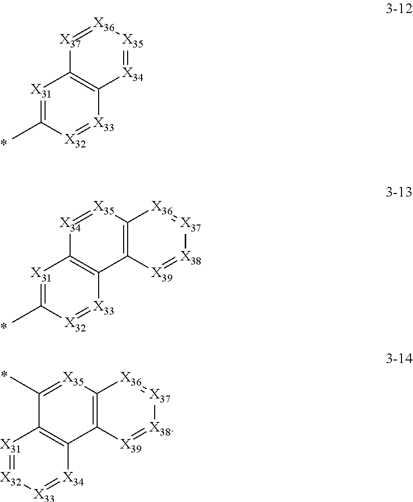

In Formulae 3-1 to 3-14, X₃₁ may be N or C(R₃₁), X₃₂ may be N or C(R₃₂), X₃₃ may be N or C(R₃₃), X₃₄ may be N or C(R₃₄), X₃₅ may be N or C(R₃₅), X₃₆ may be N or C(R₃₆), X₃₇ may be N or C(R₃₇), X₃₈ may be N or C(R₃₈), and X₃₉ may be N or C(R₃₉),

X₄₁ in Formulae 3-1, 3-2, and 3-4 to 3-9 may be N(R₄₁), C(R₄₂)(R₄₃), O, or S,

at least one of X₃₁ to X₃₃ in Formulae 3-1 and 3-2 may be N, at least one of X₃₁ to X₃₄ in Formula 3-3 may be N, at least one of X₃₁ to X₃₅ in Formulae 3-4, 3-5, and 3-10 may be N, at least one of X₃₁ to X₃₇ in Formulae 3-6 to 3-9, 3-11, and 3-12 may be N, and at least one of X₃₁ to X₃₉ in Formulae 3-13 and 3-14 may be N,

R₃₁ to R₃₉ and R₄₁ to R₄₃ may each independently be selected from hydrogen, deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a (C₁-C₂₀ alkyl)phenyl group, a di(C₁-C₂₀ alkyl)phenyl group, a (C₆-C₂₀ aryl)phenyl group, a di(C₆-C₂₀ aryl)phenyl group, a (C₃-C₂₀ heteroaryl)phenyl group, a di(C₃-C₂₀ heteroaryl)phenyl group, a pyridinyl group, a (C₁-C₂₀ alkyl)pyridinyl group, a di(C₁-C₂₀ alkyl)pyridinyl group, a (C₆-C₂₀ aryl)pyridinyl group, a di(C₆-C₂₀ aryl)pyridinyl group, a (C₃-C₂₀ heteroaryl)pyridinyl group, a di(C₃-C₂₀ heteroaryl)pyridinyl group, a pyrimidinyl group, a (C₁-C₂₀ alkyl)pyrimidinyl group, a di(C₁-C₂₀ alkyl)pyrimidinyl group, a (C₆-C₂₀ aryl)pyrimidinyl group, a di(C₆-C₂₀ aryl)pyrimidinyl group, a (C₃-C₂₀ heteroaryl)pyrimidinyl group, a di(C₃-C₂₀ heteroaryl)pyrimidinyl group, a triazinyl group, a (C₁-C₂₀ alkyl)triazinyl group, a di(C₁-C₂₀ alkyl)triazinyl group, a (C₆-C₂₀ aryl)triazinyl group, a di(C₆-C₂₀ aryl)triazinyl group, a (C₃-C₂₀ heteroaryl)triazinyl group, and a di(C₃-C₂₀ heteroaryl)triazinyl group, and

“*” indicates a binding site to a neighboring atom.

For example, R₃₁ to R₃₉ and R₄₁ to R₄₃ may each independently be selected from hydrogen, deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a (C₁-C₁₀ alkyl)phenyl group, a di(C₁-C₁₀ alkyl)phenyl group, a biphenyl group, a di(phenyl)phenyl group, a (pyridinyl)phenyl group, a di(pyridinyl)phenyl group, a (pyrimidinyl)phenyl group, a di(pyrimidinyl)phenyl group, a (triazinyl)phenyl group, a di(triazinyl)phenyl group, a pyridinyl group, a (C₁-C₁₀ alkyl)pyridinyl group, a di(C₁-C₁₀ alkyl)pyridinyl group, a (phenyl)pyridinyl group, a di(phenyl)pyridinyl group, a (pyridinyl)pyridinyl group, a di(pyridinyl)pyridinyl group, a (pyrimidinyl)pyridinyl group, a di(pyrimidinyl)pyridinyl group, a (triazinyl)pyridinyl group, a di(triazinyl)pyridinyl group, a triazinyl group, a (C₁-C₁₀ alkyl)triazinyl group, a di(C₁-C₁₀ alkyl)triazinyl group, a (phenyl)triazinyl group, a di(phenyl)triazinyl group, a (pyridinyl)triazinyl group, a di(pyridinyl)triazinyl group, a (pyrimidinyl)triazinyl group, a di(pyrimidinyl)triazinyl group, a (triazinyl)triazinyl group, and a di(triazinyl)triazinyl group, but embodiments of the present disclosure are not limited thereto.

In one or more embodiments, E₁₁ in Formula 1B may be selected from:

—CN;

a C₁-C₂₀ alkyl group substituted with at least one —CN; and

groups represented by Formulae 3-4(1) to 3-4(4), 3-5(1) to 3-5(4), 3-6(1), 3-7(1), 3-8(1), 3-9(1), 3-10(1) to 3-10(8), 3-11(1) to 3-11(23), and 3-12(1) to 3-12(23),

but embodiments of the present disclosure are not limited thereto:

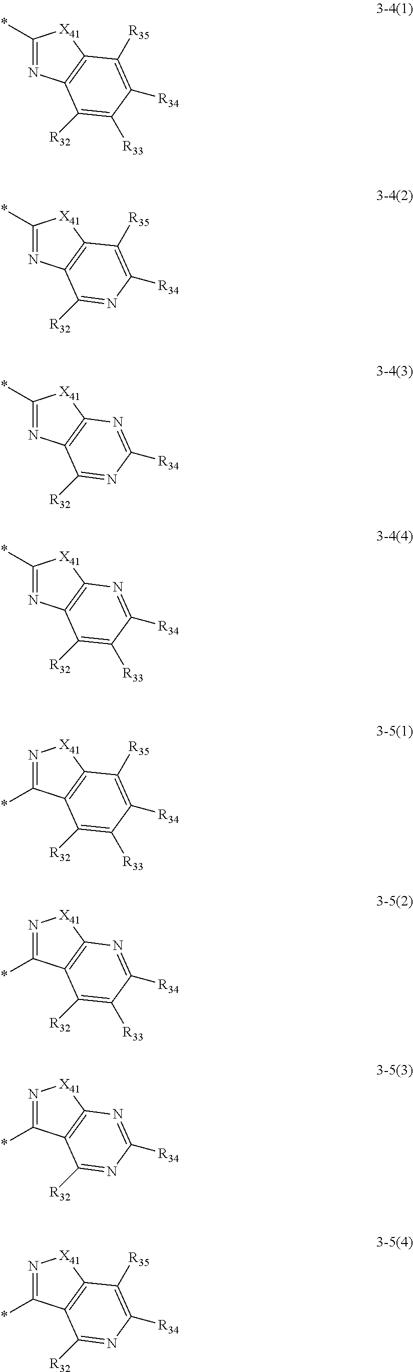

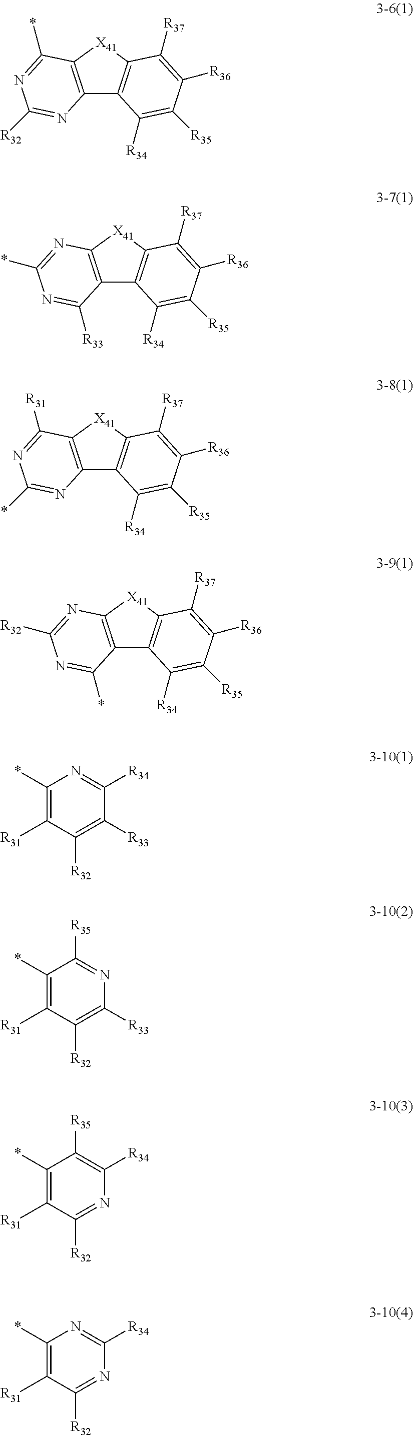

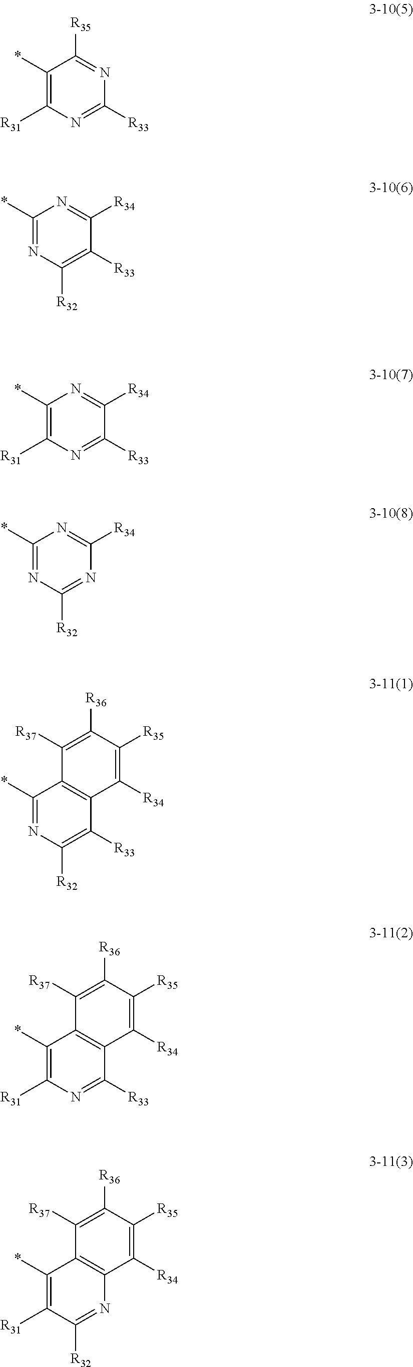

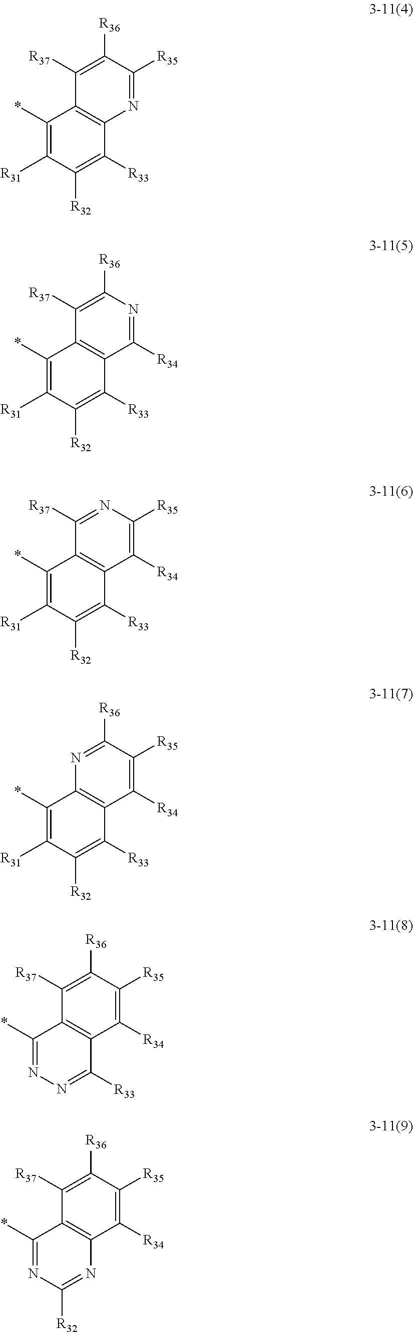

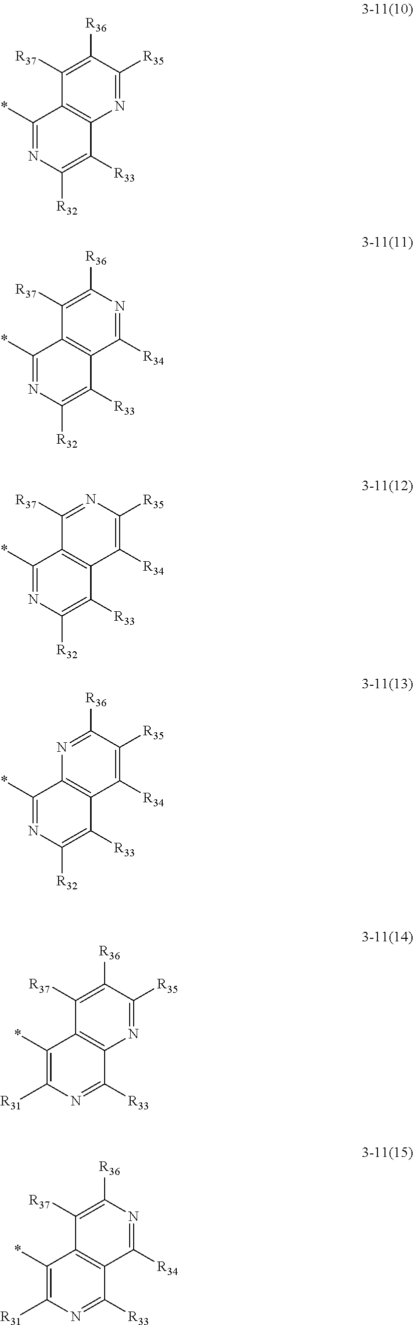

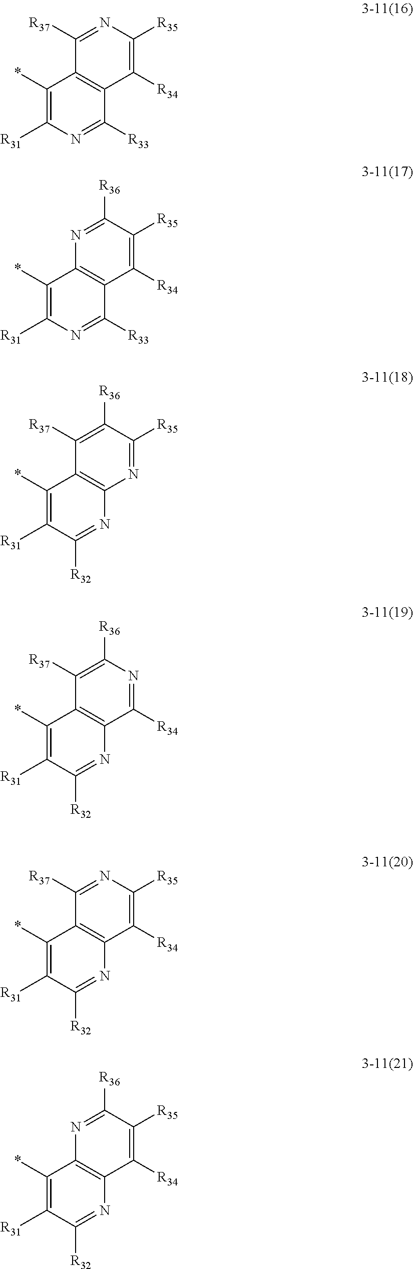

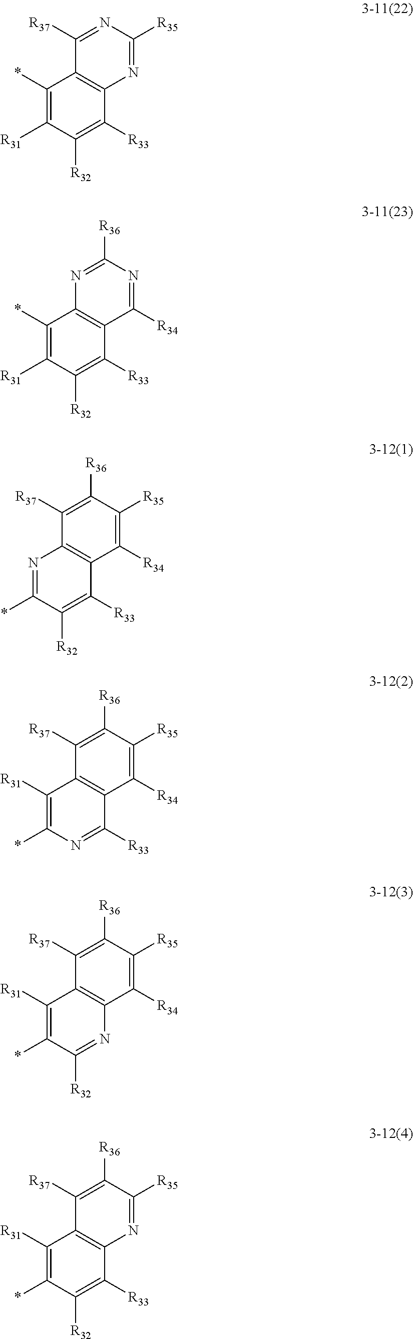

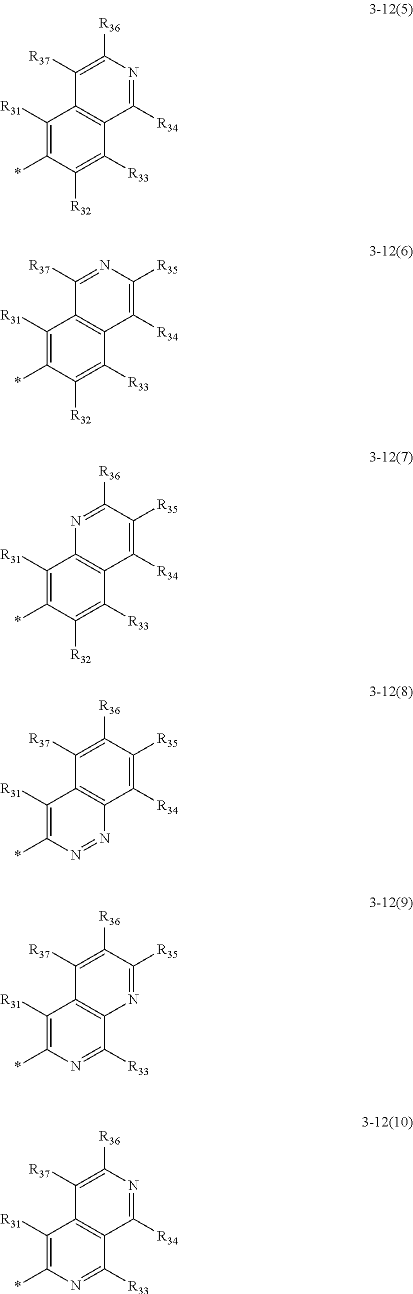

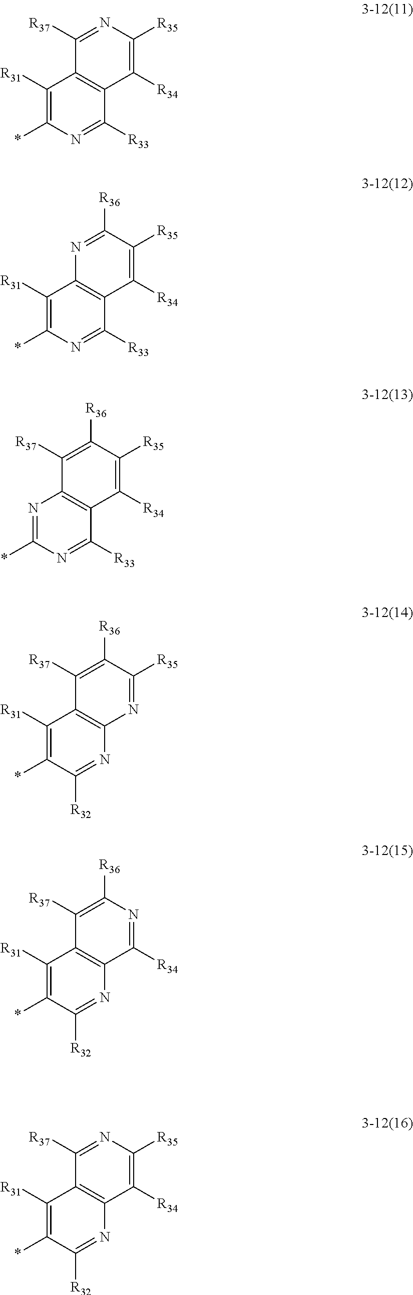

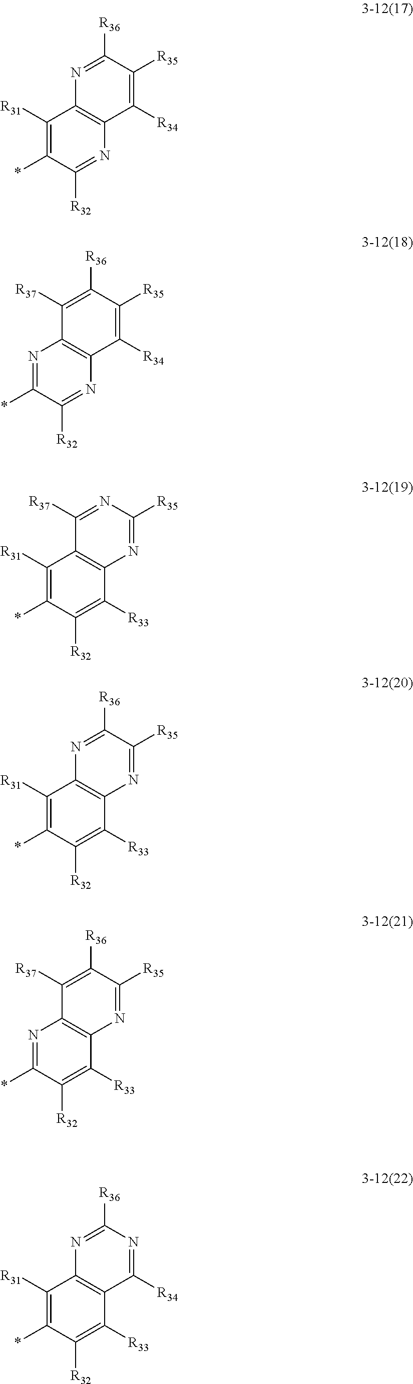

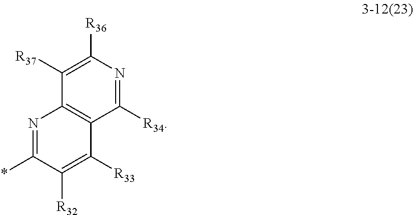

In Formulae 3-4(1) to 3-4(4), 3-5(1) to 3-5(4), 3-6(1), 3-7(1), 3-8(1), 3-9(1), 3-10(1) to 3-10(8), 3-11(1) to 3-11(23), and 3-12(1) to 3-12(23), X₄₁ may be N(R₄₁), C(R₄₂)(R₄₃), O, or S,

R₃₁ to R₃₇ and R₄₁ to R₄₃ are each independently the same as described herein, and

“*” indicates a binding site to a neighboring atom.

b11 in Formula 1B indicates the number of groups E₁₁ and may be an integer from 1 to 5, wherein, when b11 is two or more, two or more groups E₁₁ may be identical to or different from each other.

In one or more embodiments, b11 may be 1, 2, or 3, but embodiments of the present disclosure are not limited thereto.

A bond between L₁₁ and E₁₁ in Formula 1B may be a carbon-carbon single bond or a carbon-fluorine single bond.

“*” in Formula 1B indicates a binding site to a neighboring carbon.

The electron acceptor group represented by Formula 1B may be derived from any combination of L₁₁, a₁₁, E₁₁, and b₁₁ described herein.

For example, the electron acceptor group represented by Formula 1B may be selected from —CN and groups represented by Formulae 1B-1 to 1B-30, but embodiments of the present disclosure are not limited thereto:

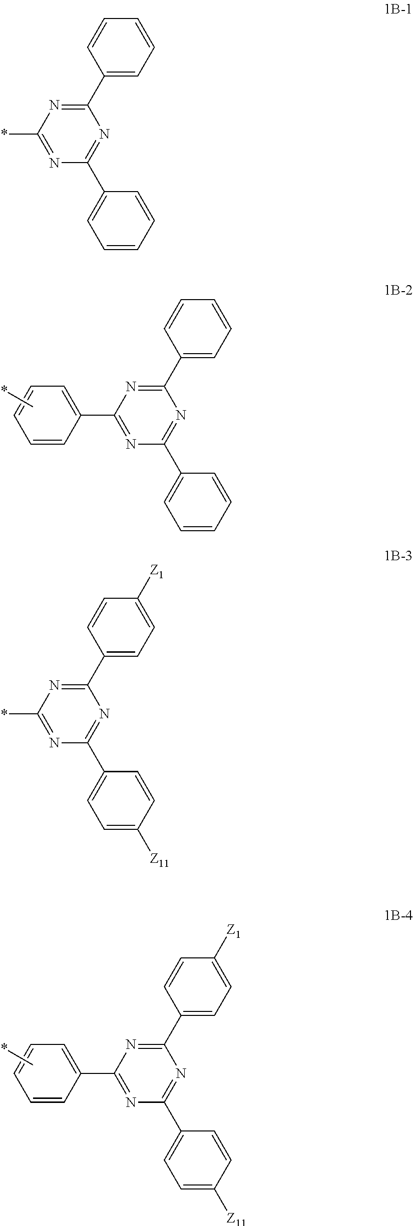

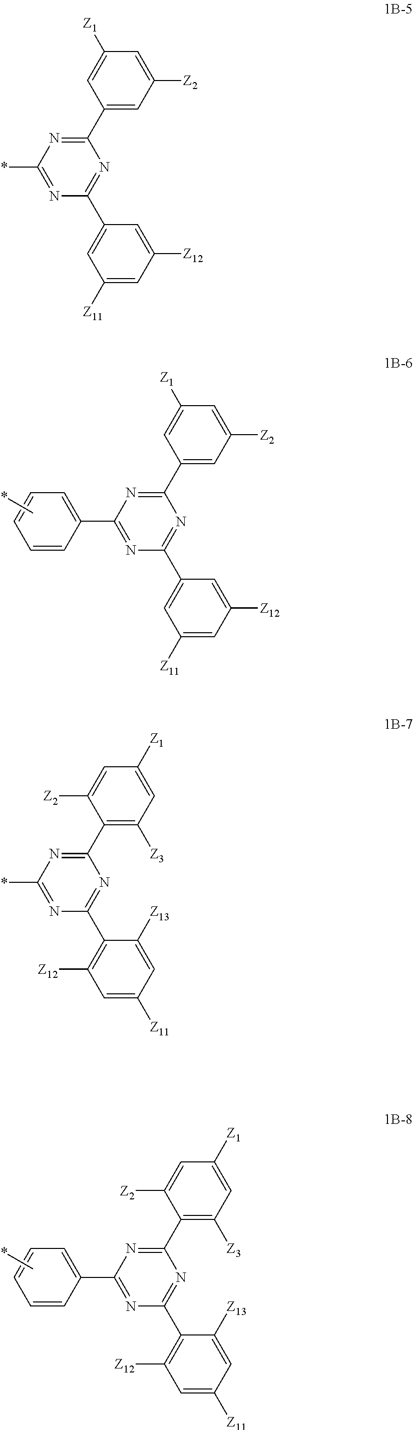

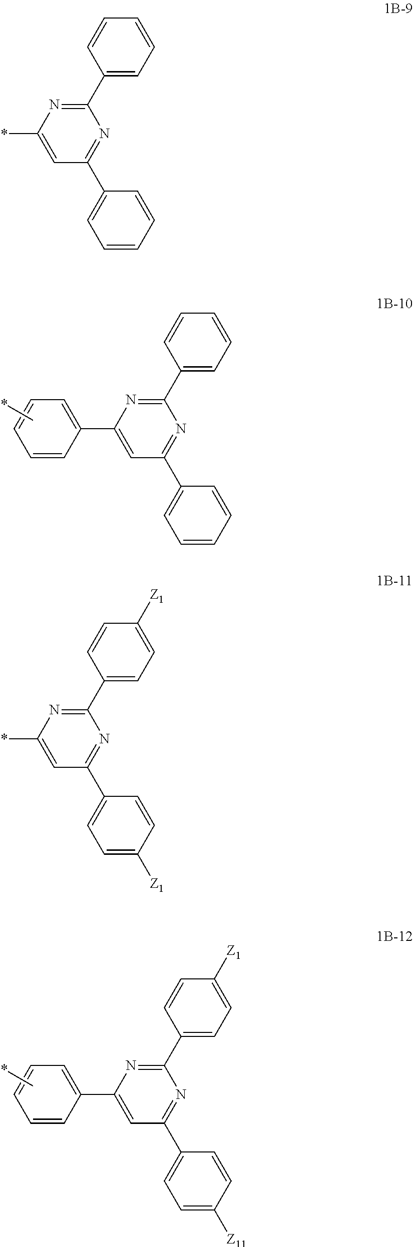

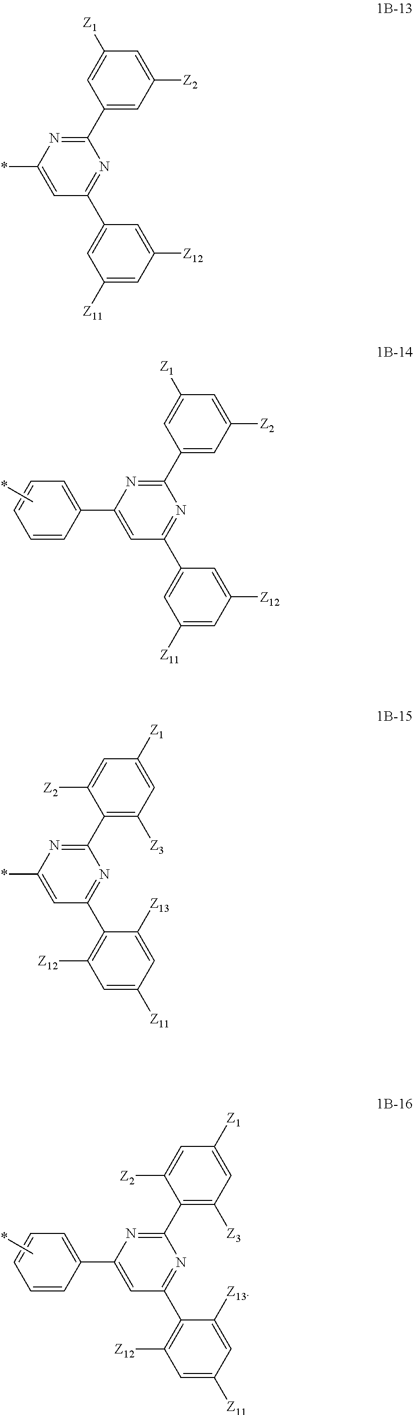

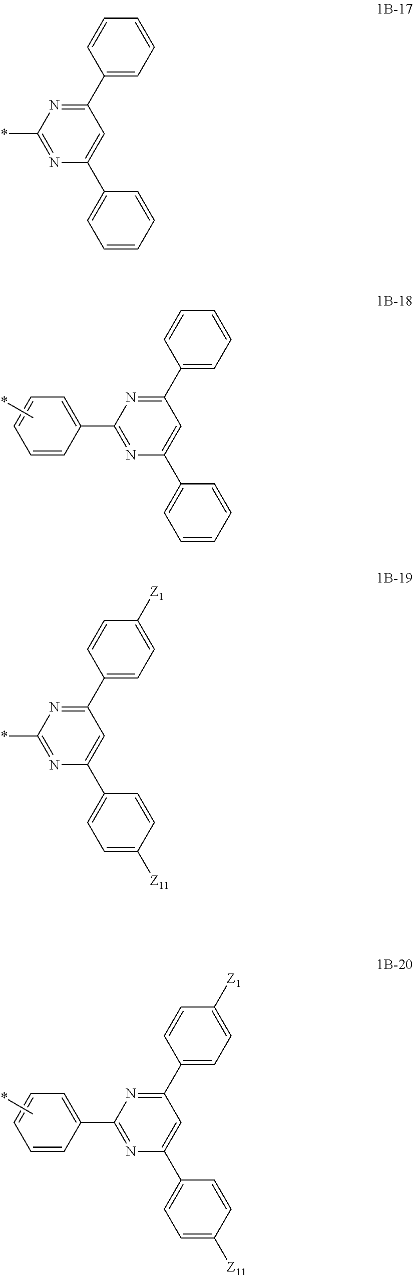

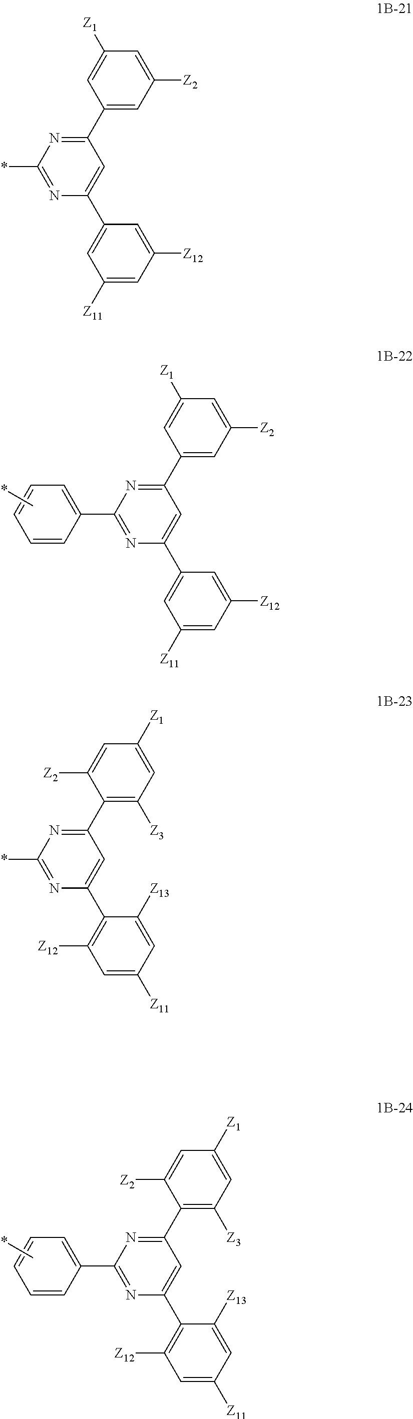

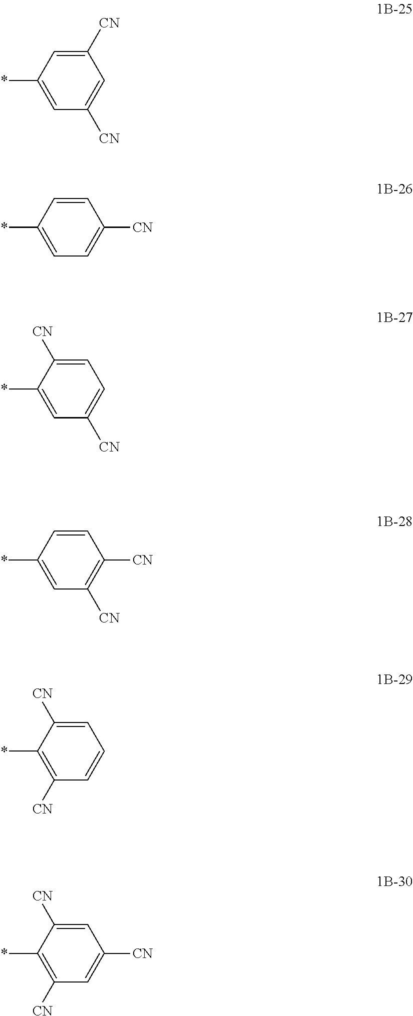 [SMILES: *c1cc(C#N)cc(C#N)c1 ; *c1ccc(C#N)cc1 ; *c1cc(C#N)ccc1C#N ; *c1ccc(C#N)c(C#N)c1 ; *c1c(C#N)cccc1C#N ; *c1c(C#N)cc(C#N)cc1C#N]

In Formulae 1B-1 to 1B-30, Z₁ to Z₃ and Z₁₁ to Z₁₃ may each independently be selected from a C₁-C₁₀ alkyl group and a phenyl group, and “*” indicates a binding site to a neighboring atom.

Since the fluorescent compound includes the electron acceptor group represented by Formula 1B as defined above, the fluorescent compound may have excellent electron transport characteristics while having a difference between a singlet excitation energy level and a triplet excitation energy level. Thus, an electronic device (for example, an organic light-emitting device) including the fluorescent compound may have both high efficiency and a long lifespan.

The fluorescent compound may be represented by one of Formulae 10-1 to 10-6:

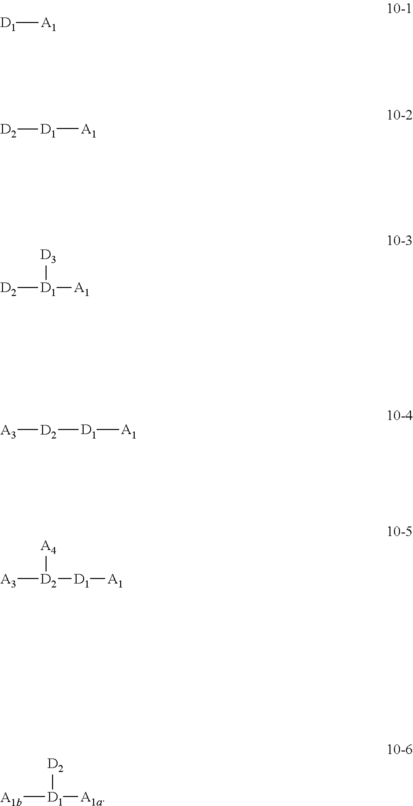

In Formulae 10-1 to 10-6,

D₁ to D₃ may each independently be selected from electron donor groups represented by Formula 1A, and

A₁, A₁ₐ, A_{1b}, A₃, and A₄ may each independently be selected from electron acceptor groups represented by Formula 1B.

In one or more embodiments, the fluorescent compound may be represented by one of Formulae 9-1 to 9-9:

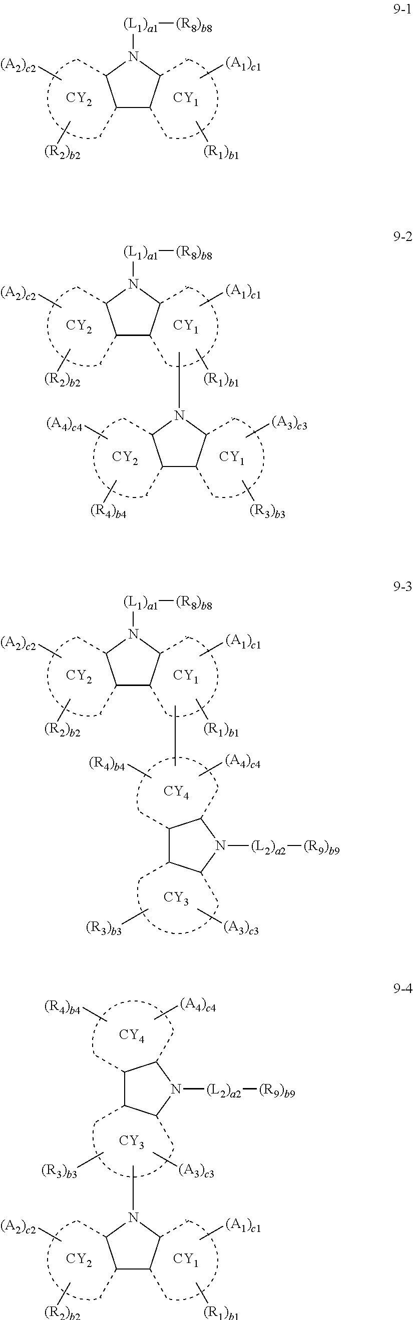

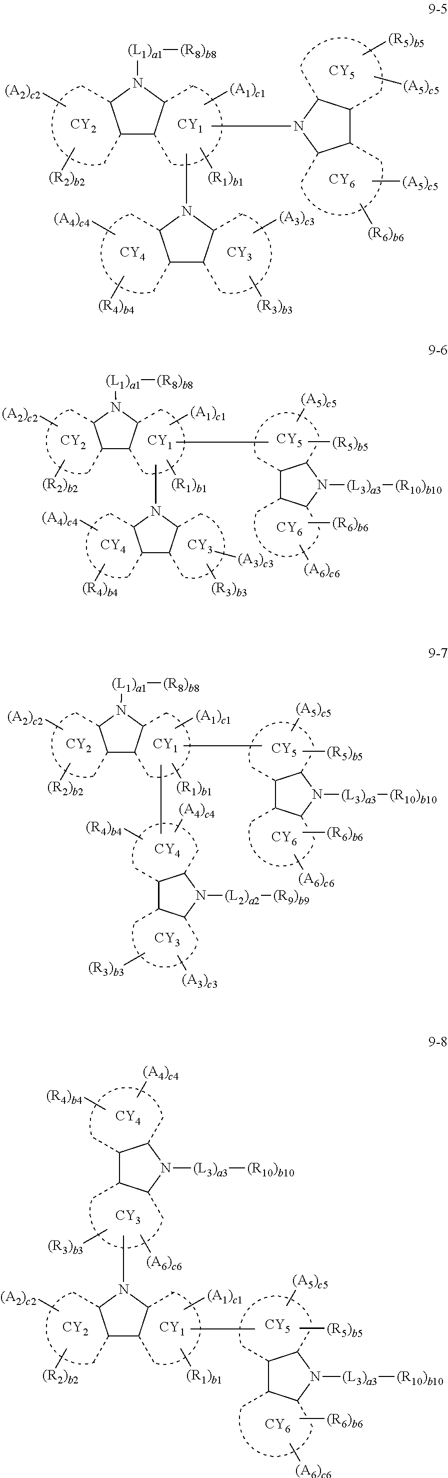

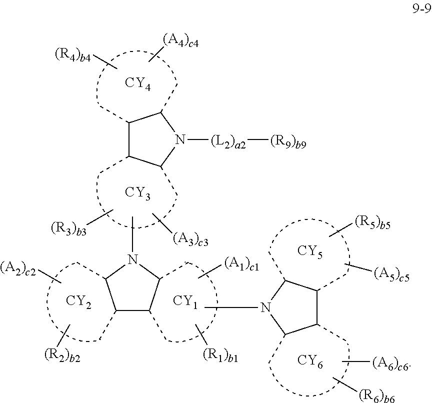

In Formulae 9-1 to 9-9,

CY₁, CY₂, R₁, R₂, b1, and b2 are the same as described herein,

L₁ to L₃ may each independently be selected from:

a single bond, a C₅-C₆₀ carbocyclic group, and a π electron-depleted nitrogen-free C₂-C₆₀ heterocyclic group; and

a C₅-C₆₀ carbocyclic group and a π electron-depleted nitrogen-free C₂-C₆₀ heterocyclic group, each substituted with at least one selected from deuterium, a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, a (C₁-C₁₀ alkyl)C₅-C₆₀ carbocyclic group, a di(C₁-C₁₀ alkyl)C₅-C₆₀ carbocyclic group, a (phenyl)C₅-C₆₀ carbocyclic group, a di(phenyl)C₅-C₆₀ carbocyclic group, a (biphenyl)C₅-C₆₀ carbocyclic group, a di(biphenyl)C₅-C₆₀ carbocyclic group, a π electron-depleted nitrogen-free C₂-C₆₀ heterocyclic group, a (C₁-C₁₀ alkyl) π electron-depleted nitrogen-free C₂-C₆₀ heterocyclic group, a di(C₁-C₁₀ alkyl) π electron-depleted nitrogen-free C₂-C₆₀ heterocyclic group, a (phenyl) π electron-depleted nitrogen-free C₂-C₆₀ heterocyclic group, a di(phenyl) π electron-depleted nitrogen-free C₂-C₆₀ heterocyclic group, a (biphenyl) π electron-depleted nitrogen-free C₂-C₆₀ heterocyclic group, and a di(biphenyl) π electron-depleted nitrogen-free C₂-C₆₀ heterocyclic group,

a1 to a3 may each independently be an integer from 1 to 3,

R₈ to R₁₀ are the same as described in connection with R₁, provided that R₈ to R₁₀ are not hydrogen,

b8 to b10 may each independently be an integer from 1 to 5,

R₁ to R₆ are the same as described in connection with R₁,

b1 to b6 are the same as described in connection with b1,

A₁ to A₆ may each independently be selected from electron acceptor groups represented by Formula 1B,

c1 to c6 may each independently be 0, 1, 2, or 3, provided that the sum of c1 and c2 in Formula 9-1 is one or more, the sum of c1 to c4 in Formulae 9-2 to 9-4 is one or more, and the sum of c1 to c6 in Formulae 9-4 to 9-6 is one or more, and

a bond between A₁ and CY₁, a bond between A₂ and CY₂, a bond between A₃ and CY₃, a bond between A₄ and CY₄, a bond between A₅ and CY₅, and a bond between A₆ and CY₆ may each be a carbon-carbon single bond.

a1, a2, and a3 in Formulae 9-1 to 9-9 respectively indicate the number of groups L₁, the number of groups L₂, and the number of groups L₃ and may each independently be an integer from 1 to 3, wherein, when a1 is two or more, two or more groups L₁ may be identical to or different from each other, when a2 is two or more, two or more groups L₂ may be identical to or different from each other, and when a3 is two or more, two or more groups L₃ may be identical to or different from each other.

For example, in Formulae 9-1 to 9-9,

L₁ to L₃ may each independently be selected from:

a single bond, a benzene group, a naphthalene group, a fluorene group, a carbazole group, a dibenzofuran group, and a dibenzothiophene group; and

a benzene group, a naphthalene group, a fluorene group, a carbazole group, a dibenzofuran group, and a dibenzothiophene group, each substituted with at least one selected from deuterium, a C₁-C₂₀ alkyl group, a C₁-C₂₀ alkoxy group, a phenyl group, a biphenyl group, a terphenyl group, a naphthyl group, a fluorenyl group, a dimethylfluorenyl group, a diphenylfluorenyl group, a carbazolyl group, a phenylcarbazolyl group, a biphenylcarbazolyl group, a dibenzofuranyl group, and a dibenzothiophenyl group,

R₁ to R₆ and R₈ to R₁₀ may each independently be selected from hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, a di(phenyl)phenyl group, a fluorenyl group, a (C₁-C₁₀ alkyl)fluorenyl group, a di(C₁-C₁₀ alkyl)fluorenyl group, a (phenyl)fluorenyl group, a di(phenyl)fluorenyl group, a carbazolyl group, a (C₁-C₁₀ alkyl)carbazolyl group, a di(C₁-C₁₀ alkyl)carbazolyl group, a (phenyl)carbazolyl group, a di(phenyl)carbazolyl group, a dibenzofuranyl group, a (C₁-C₁₀ alkyl)dibenzofuranyl group, a di(C₁-C₁₀ alkyl)dibenzofuranyl group, a (phenyl)dibenzofuranyl group, a di(phenyl)dibenzofuranyl group, a dibenzothiophenyl group, a (C₁-C₁₀ alkyl)dibenzothiophenyl group, a di(C₁-C₁₀ alkyl)dibenzothiophenyl group, a (phenyl)dibenzothiophenyl group, a di(phenyl)dibenzothiophenyl group, a di(phenyl)dibenzophenyl group, an indolofluorenyl group, a (C₁-C₁₀ alkyl)indolofluorenyl group, a di(C₁-C₁₀ alkyl)indolofluorenyl group, a (phenyl)indolofluorenyl group, a di(phenyl)indolofluorenyl group, an indolocarbazolyl group, a (C₁-C₁₀ alkyl)indolocarbazolyl group, a di(C₁-C₁₀ alkyl)indolocarbazolyl group, a (phenyl)indolocarbazolyl group, a di(phenyl)indolocarbazolyl group, an indolodibenzofuranyl group, a (C₁-C₁₀ alkyl)indolodibenzofuranyl group, a di(C₁-C₁₀ alkyl)indolodibenzofuranyl group, a (phenyl)indolodibenzofuranyl group, a di(phenyl)indolodibenzofuranyl group, an indolodibenzothiophenyl group, a (C₁-C₁₀ alkyl)indolodibenzothiophenyl group, a di(C₁-C₁₀ alkyl)indolodibenzothiophenyl group, a (phenyl)indolodibenzothiophenyl group, a di(phenyl)indolodibenzothiophenyl group and a di(phenyl)indolodibenzophenyl group, provided that R₈ to R₁₀ are not hydrogen,

b1 to b6 may each independently be 0, 1, or 2, and

b8 to b10 may each independently be 1 or 2, but embodiments of the present disclosure are not limited thereto.

In Formulae 9-1 to 9-9, a moiety represented by

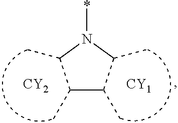

a moiety represented by

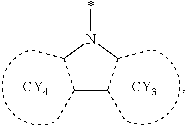

and a moiety represented by

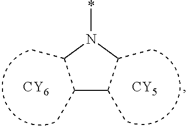

may each independently be derived from groups represented by Formulae 8-1 to 8-7, and those of ordinary skill in the art may understand from Formulae 9-1 to 9-9 and definitions thereof that at least one hydrogen of a core represented by Formulae 8-1 to 8-7 may optionally be substituted with substituents defined by Formulae 9-1 to 9-9:

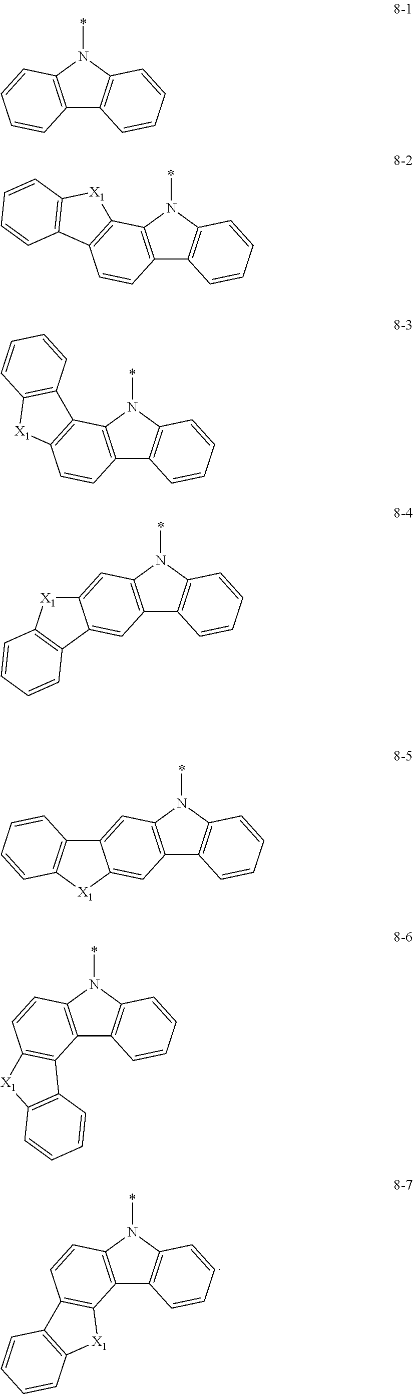

In Formulae 8-2 to 8-7, X₁ may be N(R′), C(R′)(R″), O, or S, R′ and R″ are the same as described in connection with R₁, and “*” indicates a binding site to a neighboring atom.

In an embodiment,

1) in Formula 9-1, c1 may be 1 or 2 and c2 may be 0,

2) in Formulae 9-2 to 9-4, i) c1 may be 1 or 2, c2 may be 0, and the sum of c3 and c4 may be 0, 1, or 2, or ii) c1 may be 0, c2 may be 1 or 2, and the sum of c3 and c4 may be 0, 1, or 2, and

3) in Formulae 9-5 to 9-9, i) c1 may be 1 or 2, c2 may be 0, and the sum of c3, c4, c5, and c6 may be 1 or 2, or ii) c1 may be 0, c2 may be 1 or 2, and the sum of c3, c4, c5, and c6 may be 0, 1, or 2, but embodiments of the present disclosure are not limited thereto.

In one or more embodiments, the fluorescent compound may be represented by one of Formulae 1-1 to 1-7:

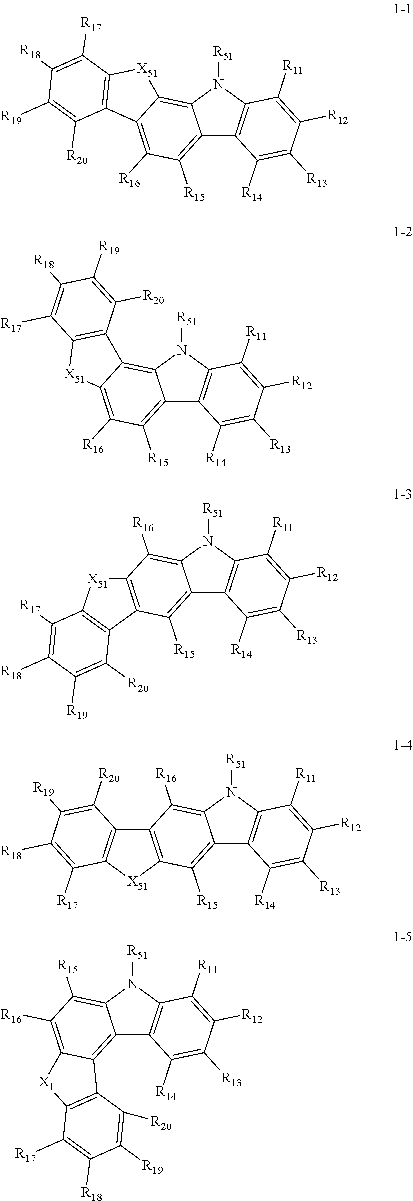

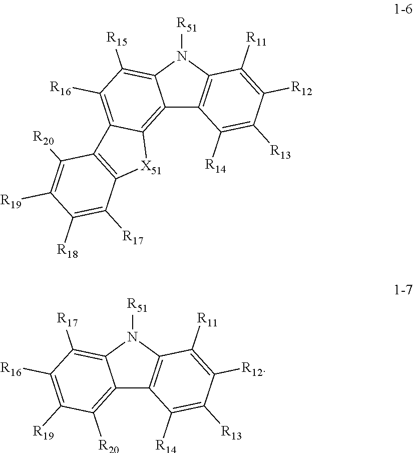

In Formulae 1-1 to 1-7,

X₅₁ may be C(R₅₂)(R₅₃), N(R₅₂), O, or S, and

1) i) R₁₁ may be an electron acceptor group represented by Formula 1B, and ii) one selected from R₁₂ to R₂₀ and R₅₁ to R₅₃ may be a group represented by one of Formulae 6-1 to 6-7, and the rest may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;

2) i) R₁₂ may be an electron acceptor group represented by Formula 1B, and ii) one selected from R₁₁, R₁₃ to R₂₀, and R₅₁ to R₅₃ may be a group represented by one selected from Formulae 6-1 to 6-7, and the rest may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;

3) i) R₁₃ may be an electron acceptor group represented by Formula 1B, and ii) one selected from R₁₁, R₁₂, R₁₄ to R₂₀, and R₅₁ to R₅₃ may be a group represented by Formulae 6-1 to 6-7, and the rest may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;

4) i) R₁₄ may be an electron acceptor group represented by Formula 1B, and ii) one selected from R₁₁ to R₁₃, R₁₅ to R₂₀, and R₅₁ to R₅₃ may be a group a group represented by one selected from Formulae 6-1 to 6-7, and the rest may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;

5) i) R₁₅ may be an electron acceptor group represented by Formula 1B, and ii) one selected from R₁₁ to R₁₄, R₁₆ to R₂₀, and R₅₁ to R₅₃ may be a group represented by one selected from Formulae 6-1 to 6-7, and the rest may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;

6) i) R₁₆ may be an electron acceptor group represented by Formula 1B, and ii) one selected from R₁₁ to R₁₅, R₁₇ to R₂₀, and R₅₁ to R₅₃ may be a group represented by one selected from Formulae 6-1 to 6-7, and the rest may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;

7) i) R₁₂ may be an electron acceptor group represented by Formula 1B, ii) R₁₁ and R₁₃ may each independently be a group represented by one selected from Formulae 6-1 to 6-7, and iii) R₁₄ to R₂₀ and R₅₁ to R₅₃ may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;

8) i) R₁₃ may be an electron acceptor group represented by Formula 1B, ii) R₁₂ and R₁₄ may each independently be a group represented by one selected from Formulae 6-1 to 6-7, and iii) R₁₁, R₁₅ to R₂₀, and R₅₁ to R₅₃ may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;

9) i) R₁₂ and R₁₃ may each independently be an electron acceptor group represented by Formula 1B, and ii) one selected from R₁₁, R₁₄ to R₂₀, and R₅₁ to R₅₃ may be a group represented by one selected from Formulae 6-1 to 6-7, and the rest may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;

10) i) R₁₁ and R₁₂ may each independently be an electron acceptor group represented by Formula 1B, and ii) one selected from R₁₃ to R₂₀ and R₅₁ to R₅₃ may be a group represented by one selected from Formulae 6-1 to 6-7, and the rest may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group; or

11) i) R₁₁ may be an electron acceptor group represented by Formula 1B, ii) R₁₂ and R₁₃ may each independently be a group represented by one selected from Formulae 6-1 to 6-7, and iii) R₁₄ to R₂₀ and R₅₁ to R₅₃ may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group.

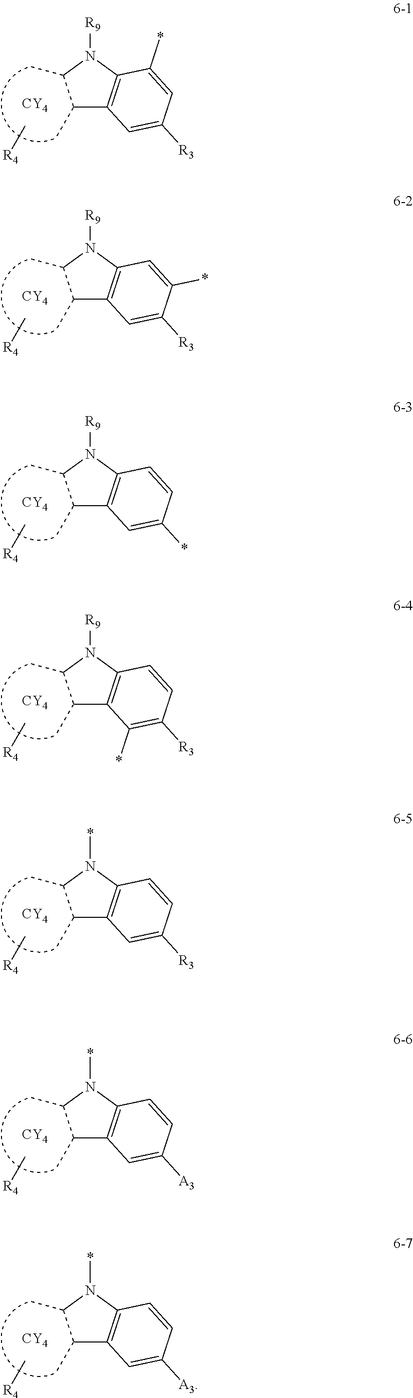

In Formulae 6-1 to 6-7,

CY₄ may be a benzene group, a fluorene group, a dimethylfluorene group, a diphenylfluorene group, a carbazole group, a phehylcarbazole group, a biphenylcarbazole group, a dibenzofuran group, or a dibenzothiophene group,

R₃, R₄, and R₉ may each independently be hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group,

A₃ and A₄ may each independently be selected from acceptor groups represented by Formula 1B, and

“*” indicates a binding site to a neighboring atom, but embodiments of the present disclosure are not limited thereto.

The fluorescent compound may be one of Compounds 1 to 936, but embodiments of the present disclosure are not limited thereto:

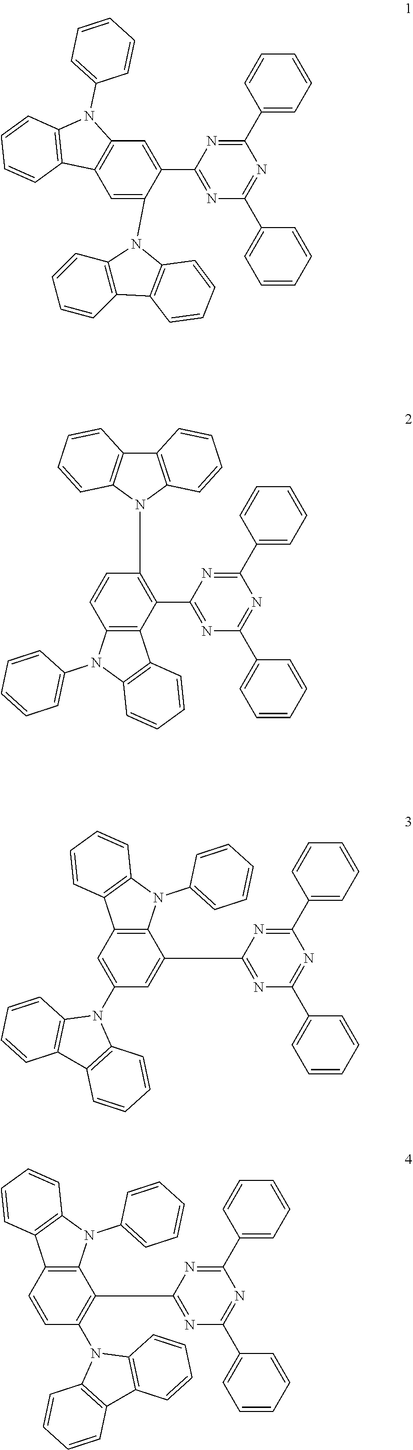 [SMILES: c1ccc(-c2nc(-c3ccccc3)nc(-c3cc4c(cc3-n3c5ccccc5c5ccccc53)c3ccccc3n4-c3ccccc3)n2)cc1 ; c1ccc(-c2nc(-c3ccccc3)nc(-c3c(-n4c5ccccc5c5ccccc54)ccc4c3c3ccccc3n4-c3ccccc3)n2)cc1 ; c1ccc(-c2nc(-c3ccccc3)nc(-c3cc(-n4c5ccccc5c5ccccc54)cc4c5ccccc5n(-c5ccccc5)c34)n2)cc1 ; c1ccc(-c2nc(-c3ccccc3)nc(-c3c(-n4c5ccccc5c5ccccc54)ccc4c5ccccc5n(-c5ccccc5)c34)n2)cc1]

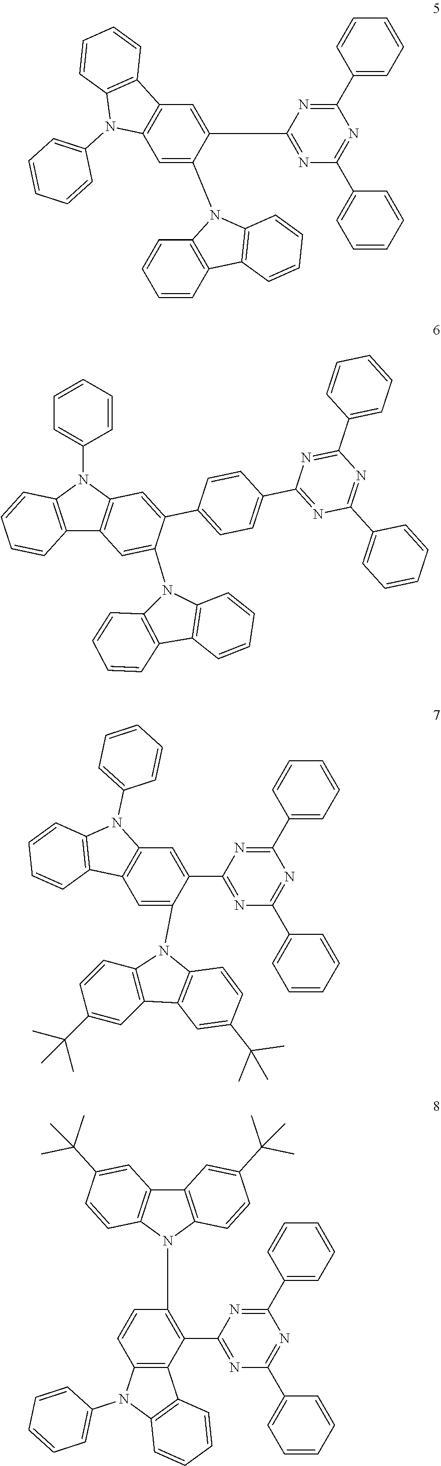

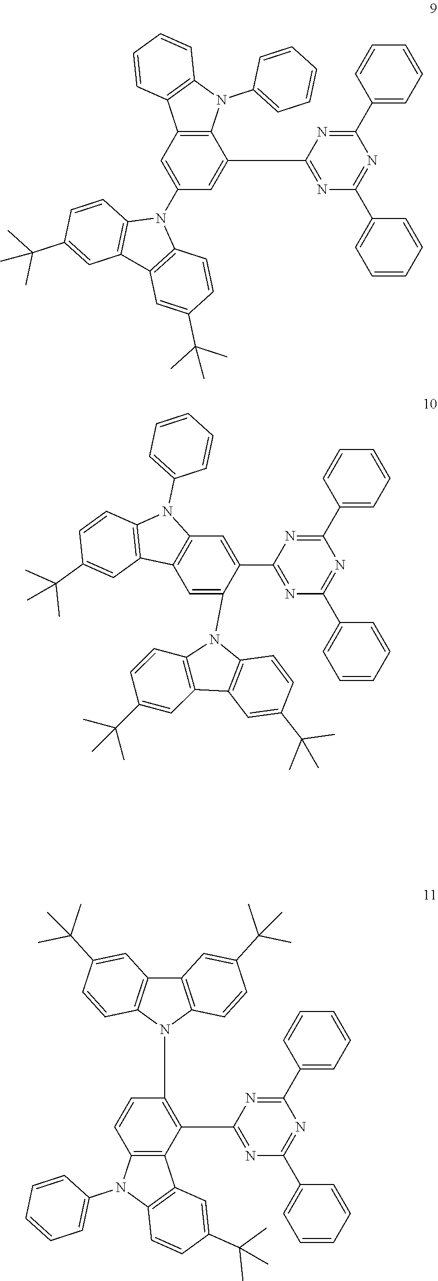

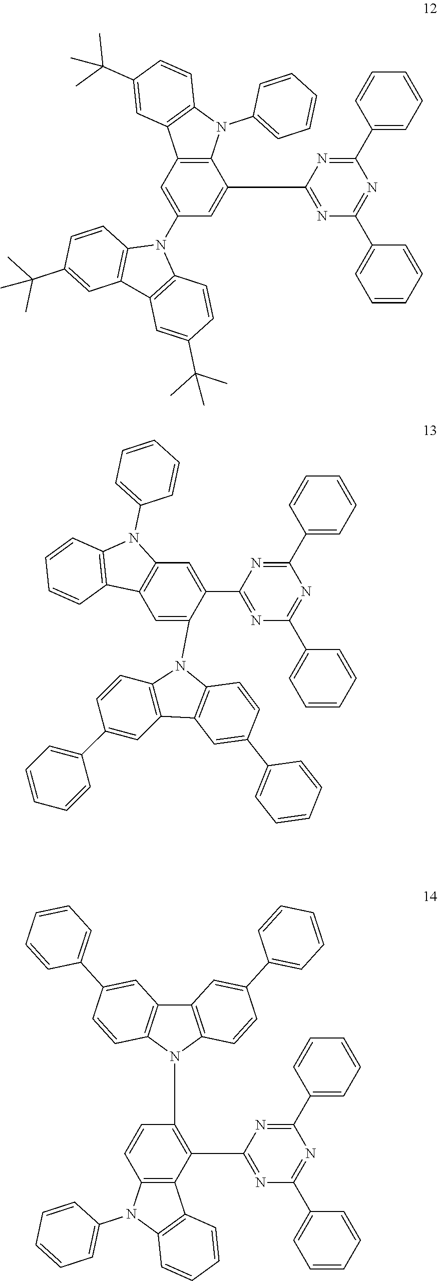 [SMILES: CC(C)(C)c1ccc2c(c1)c1cc(C(C)(C)C)ccc1n2-c1cc(-c2nc(-c3ccccc3)nc(-c3ccccc3)n2)c2c(c1)c1cc(C(C)(C)C)ccc1n2-c1ccccc1 ; c1ccc(-c2ccc3c(c2)c2cc(-c4ccccc4)ccc2n3-c2cc3c4ccccc4n(-c4ccccc4)c3cc2-c2nc(-c3ccccc3)nc(-c3ccccc3)n2)cc1 ; c1ccc(-c2ccc3c(c2)c2cc(-c4ccccc4)ccc2n3-c2ccc3c(c2-c2nc(-c4ccccc4)nc(-c4ccccc4)n2)c2ccccc2n3-c2ccccc2)cc1]

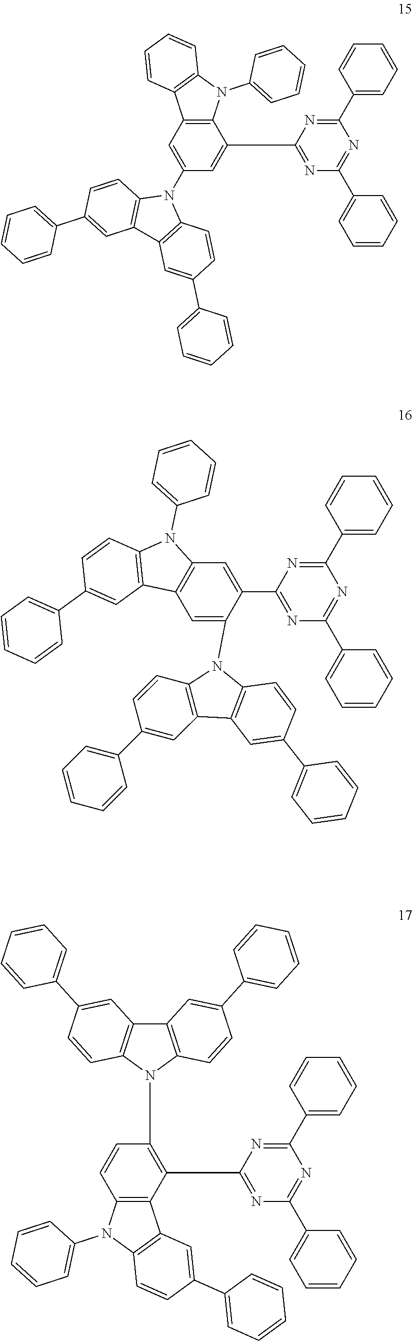 [SMILES: c1ccc(-c2ccc3c(c2)c2cc(-c4ccccc4)ccc2n3-c2cc(-c3nc(-c4ccccc4)nc(-c4ccccc4)n3)c3c(c2)c2ccccc2n3-c2ccccc2)cc1 ; c1ccc(-c2ccc3c(c2)c2cc(-n4c5ccc(-c6ccccc6)cc5c5cc(-c6ccccc6)ccc54)c(-c4nc(-c5ccccc5)nc(-c5ccccc5)n4)cc2n3-c2ccccc2)cc1 ; c1ccc(-c2ccc3c(c2)c2cc(-c4ccccc4)ccc2n3-c2ccc3c(c2-c2nc(-c4ccccc4)nc(-c4ccccc4)n2)c2cc(-c4ccccc4)ccc2n3-c2ccccc2)cc1]

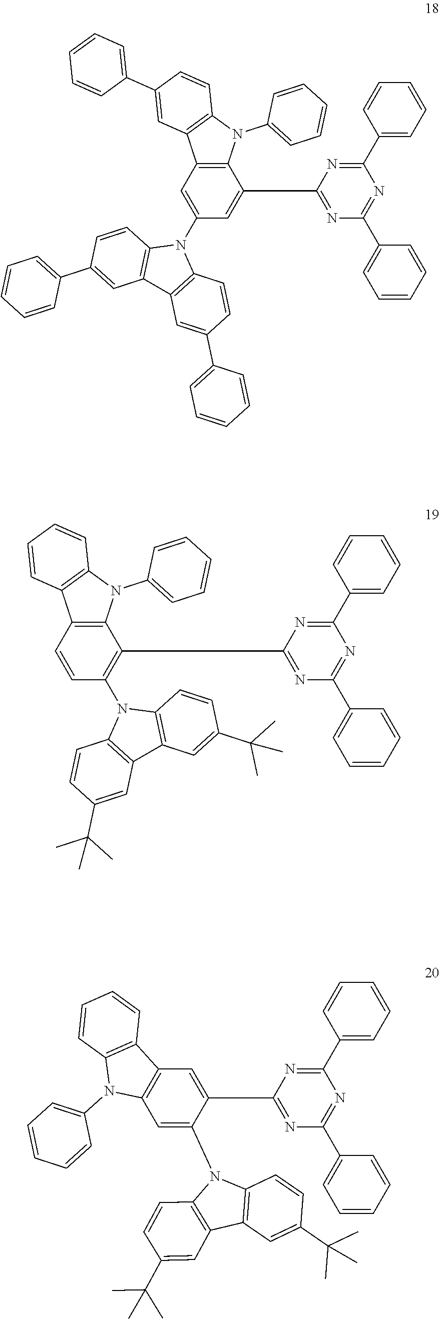 [SMILES: c1ccc(-c2ccc3c(c2)c2cc(-c4ccccc4)ccc2n3-c2cc(-c3nc(-c4ccccc4)nc(-c4ccccc4)n3)c3c(c2)c2cc(-c4ccccc4)ccc2n3-c2ccccc2)cc1 ; CC(C)(C)c1ccc2c(c1)c1cc(C(C)(C)C)ccc1n2-c1ccc2c3ccccc3n(-c3ccccc3)c2c1-c1nc(-c2ccccc2)nc(-c2ccccc2)n1 ; CC(C)(C)c1ccc2c(c1)c1cc(C(C)(C)C)ccc1n2-c1cc2c(cc1-c1nc(-c3ccccc3)nc(-c3ccccc3)n1)c1ccccc1n2-c1ccccc1]

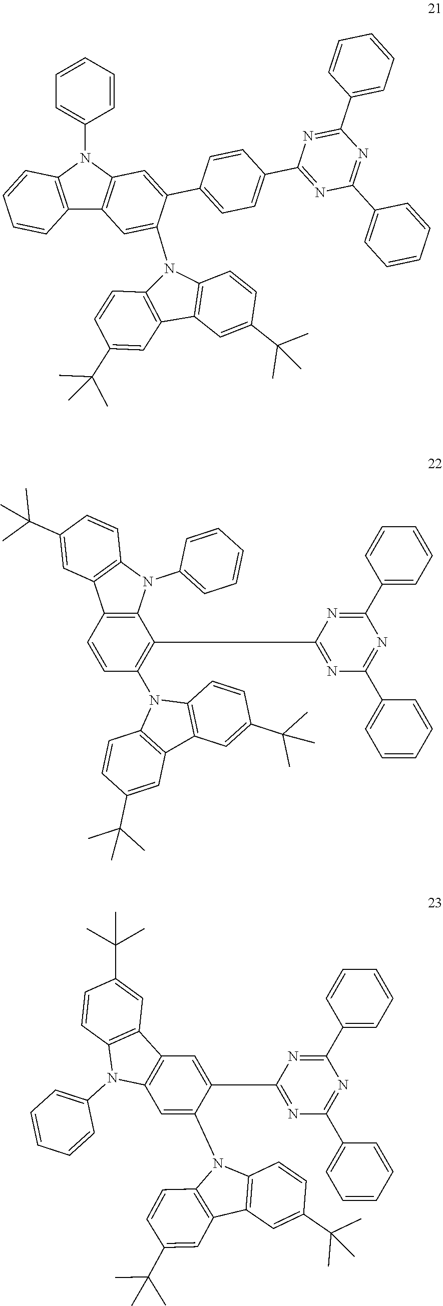 [SMILES: CC(C)(C)c1ccc2c(c1)c1cc(C(C)(C)C)ccc1n2-c1cc2c3ccccc3n(-c3ccccc3)c2cc1-c1ccc(-c2nc(-c3ccccc3)nc(-c3ccccc3)n2)cc1 ; CC(C)(C)c1ccc2c(c1)c1cc(C(C)(C)C)ccc1n2-c1ccc2c3cc(C(C)(C)C)ccc3n(-c3ccccc3)c2c1-c1nc(-c2ccccc2)nc(-c2ccccc2)n1 ; CC(C)(C)c1ccc2c(c1)c1cc(-c3nc(-c4ccccc4)nc(-c4ccccc4)n3)c(-n3c4ccc(C(C)(C)C)cc4c4cc(C(C)(C)C)ccc43)cc1n2-c1ccccc1]

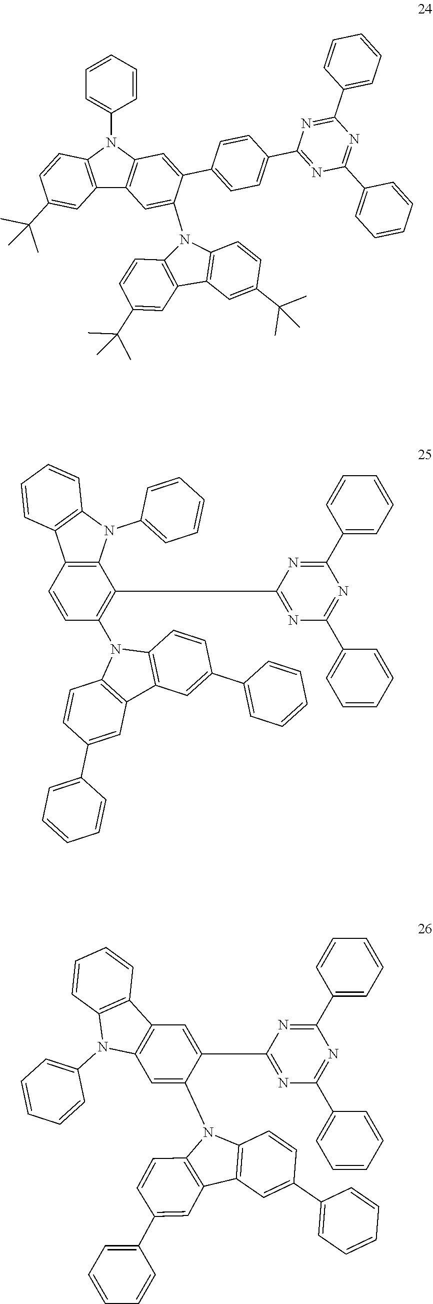 [SMILES: CC(C)(C)c1ccc2c(c1)c1cc(-n3c4ccc(C(C)(C)C)cc4c4cc(C(C)(C)C)ccc43)c(-c3ccc(-c4nc(-c5ccccc5)nc(-c5ccccc5)n4)cc3)cc1n2-c1ccccc1 ; c1ccc(-c2ccc3c(c2)c2cc(-c4ccccc4)ccc2n3-c2ccc3c4ccccc4n(-c4ccccc4)c3c2-c2nc(-c3ccccc3)nc(-c3ccccc3)n2)cc1 ; c1ccc(-c2ccc3c(c2)c2cc(-c4ccccc4)ccc2n3-c2cc3c(cc2-c2nc(-c4ccccc4)nc(-c4ccccc4)n2)c2ccccc2n3-c2ccccc2)cc1]

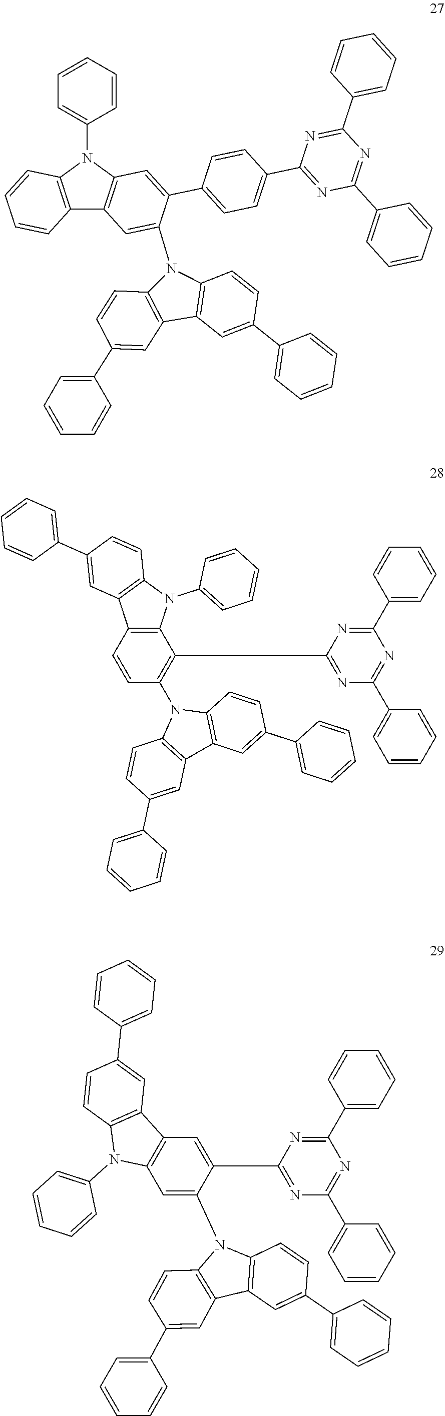 [SMILES: c1ccc(-c2ccc3c(c2)c2cc(-c4ccccc4)ccc2n3-c2cc3c4ccccc4n(-c4ccccc4)c3cc2-c2ccc(-c3nc(-c4ccccc4)nc(-c4ccccc4)n3)cc2)cc1 ; c1ccc(-c2ccc3c(c2)c2cc(-c4ccccc4)ccc2n3-c2ccc3c4cc(-c5ccccc5)ccc4n(-c4ccccc4)c3c2-c2nc(-c3ccccc3)nc(-c3ccccc3)n2)cc1 ; c1ccc(-c2ccc3c(c2)c2cc(-c4nc(-c5ccccc5)nc(-c5ccccc5)n4)c(-n4c5ccc(-c6ccccc6)cc5c5cc(-c6ccccc6)ccc54)cc2n3-c2ccccc2)cc1]

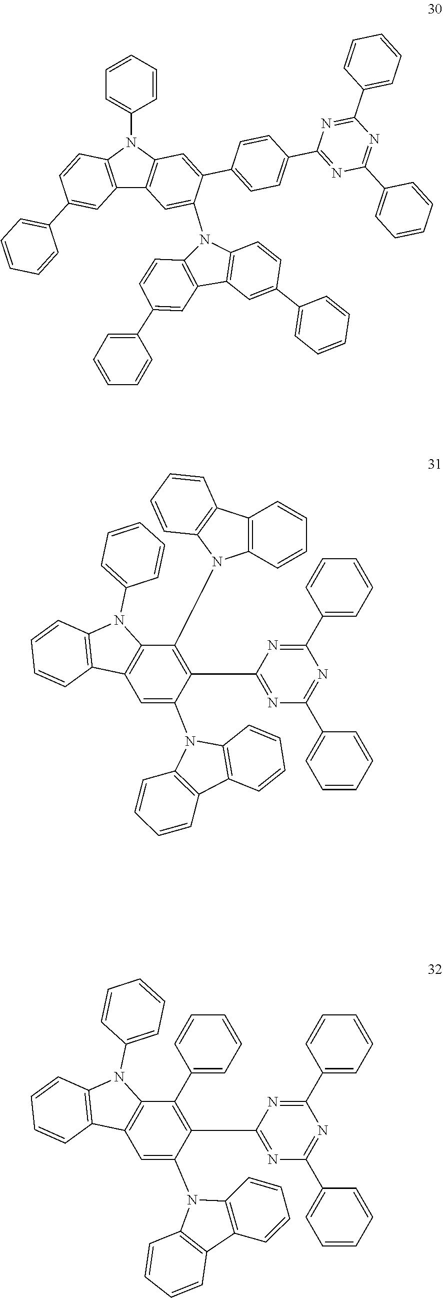 [SMILES: c1ccc(-c2ccc3c(c2)c2cc(-n4c5ccc(-c6ccccc6)cc5c5cc(-c6ccccc6)ccc54)c(-c4ccc(-c5nc(-c6ccccc6)nc(-c6ccccc6)n5)cc4)cc2n3-c2ccccc2)cc1 ; c1ccc(-c2nc(-c3ccccc3)nc(-c3c(-n4c5ccccc5c5ccccc54)cc4c5ccccc5n(-c5ccccc5)c4c3-n3c4ccccc4c4ccccc43)n2)cc1 ; c1ccc(-c2nc(-c3ccccc3)nc(-c3c(-n4c5ccccc5c5ccccc54)cc4c5ccccc5n(-c5ccccc5)c4c3-c3ccccc3)n2)cc1]

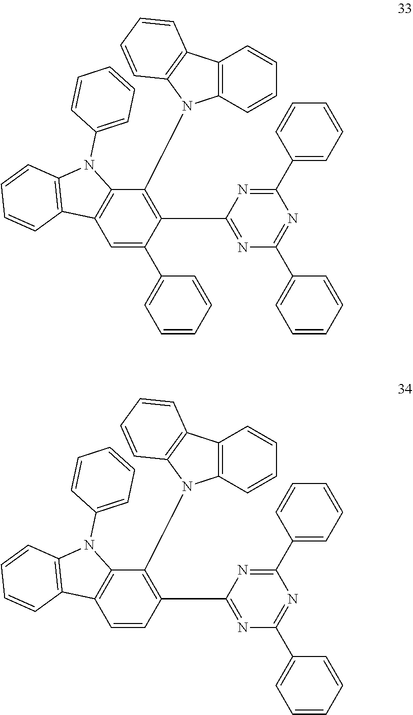 [SMILES: c1ccc(-c2nc(-c3ccccc3)nc(-c3c(-c4ccccc4)cc4c5ccccc5n(-c5ccccc5)c4c3-n3c4ccccc4c4ccccc43)n2)cc1 ; c1ccc(-c2nc(-c3ccccc3)nc(-c3ccc4c5ccccc5n(-c5ccccc5)c4c3-n3c4ccccc4c4ccccc43)n2)cc1]

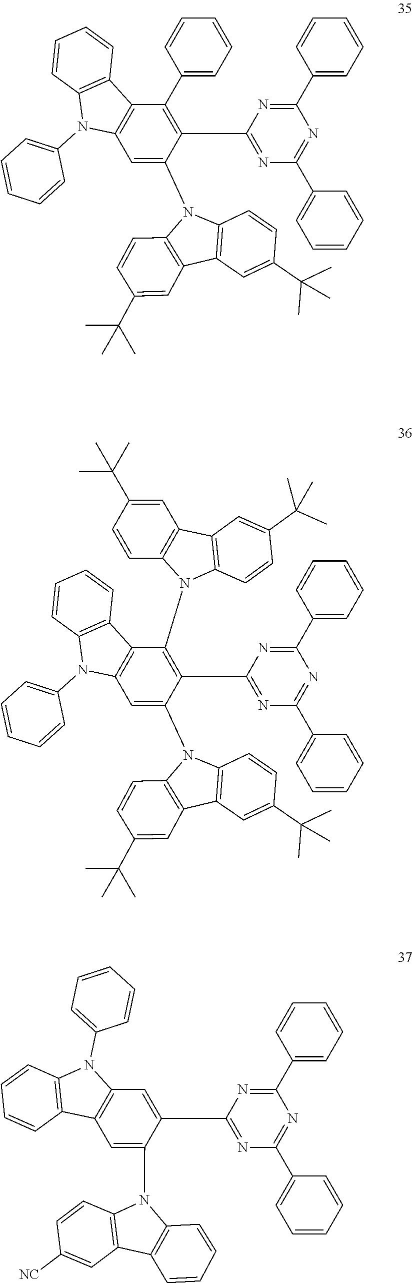 [SMILES: CC(C)(C)c1ccc2c(c1)c1cc(C(C)(C)C)ccc1n2-c1cc2c(c(-c3ccccc3)c1-c1nc(-c3ccccc3)nc(-c3ccccc3)n1)c1ccccc1n2-c1ccccc1 ; CC(C)(C)c1ccc2c(c1)c1cc(C(C)(C)C)ccc1n2-c1cc2c(c(-n3c4ccc(C(C)(C)C)cc4c4cc(C(C)(C)C)ccc43)c1-c1nc(-c3ccccc3)nc(-c3ccccc3)n1)c1ccccc1n2-c1ccccc1 ; N#Cc1ccc2c(c1)c1ccccc1n2-c1cc2c3ccccc3n(-c3ccccc3)c2cc1-c1nc(-c2ccccc2)nc(-c2ccccc2)n1]

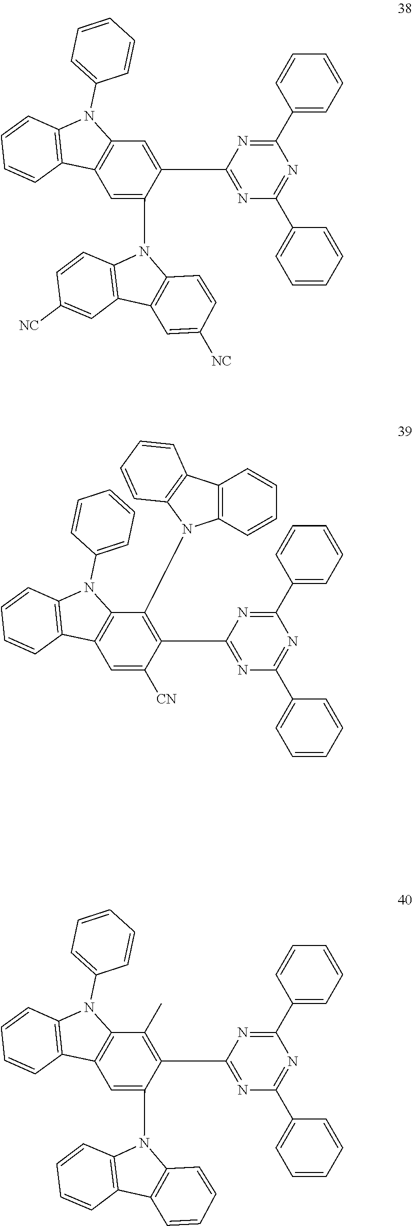

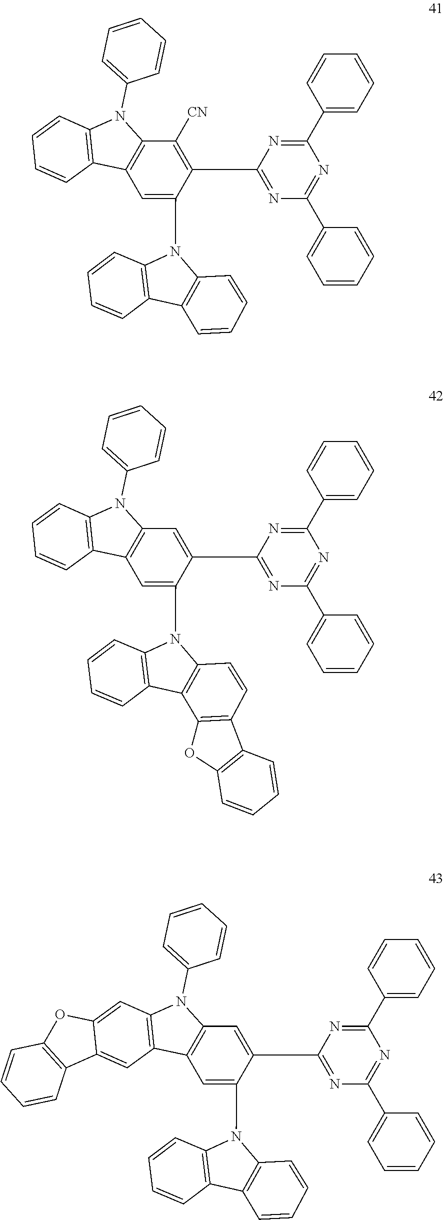 [SMILES: N#Cc1c(-c2nc(-c3ccccc3)nc(-c3ccccc3)n2)c(-n2c3ccccc3c3ccccc32)cc2c3ccccc3n(-c3ccccc3)c12 ; c1ccc(-c2nc(-c3ccccc3)nc(-c3cc4c(cc3-n3c5ccccc5c5c6oc7ccccc7c6ccc53)c3ccccc3n4-c3ccccc3)n2)cc1 ; c1ccc(-c2nc(-c3ccccc3)nc(-c3cc4c(cc3-n3c5ccccc5c5ccccc53)c3cc5c(cc3n4-c3ccccc3)oc3ccccc35)n2)cc1]

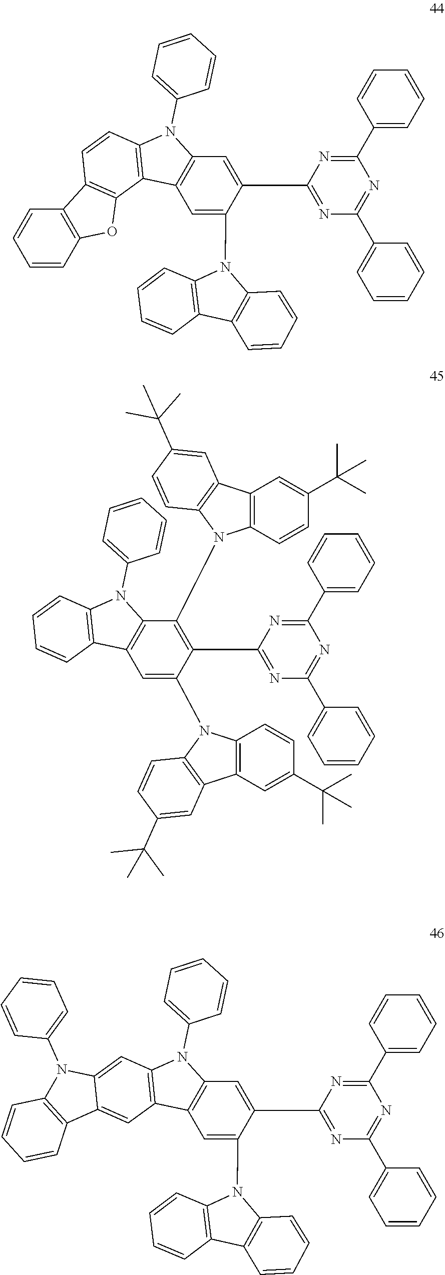 [SMILES: c1ccc(-c2nc(-c3ccccc3)nc(-c3cc4c(cc3-n3c5ccccc5c5ccccc53)c3c5oc6ccccc6c5ccc3n4-c3ccccc3)n2)cc1 ; CC(C)(C)c1ccc2c(c1)c1cc(C(C)(C)C)ccc1n2-c1cc2c3ccccc3n(-c3ccccc3)c2c(-n2c3ccc(C(C)(C)C)cc3c3cc(C(C)(C)C)ccc32)c1-c1nc(-c2ccccc2)nc(-c2ccccc2)n1 ; c1ccc(-c2nc(-c3ccccc3)nc(-c3cc4c(cc3-n3c5ccccc5c5ccccc53)c3cc5c6ccccc6n(-c6ccccc6)c5cc3n4-c3ccccc3)n2)cc1]

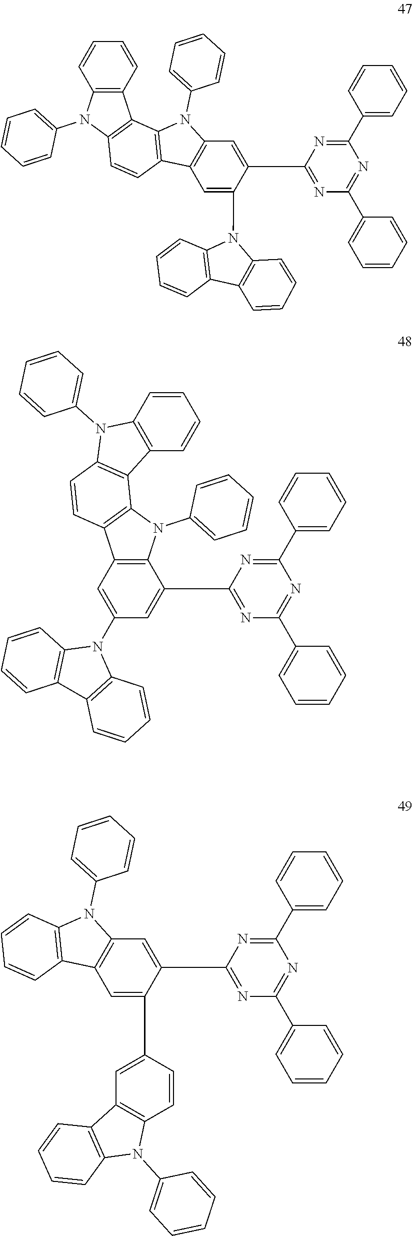 [SMILES: c1ccc(-c2nc(-c3ccccc3)nc(-c3cc4c(cc3-n3c5ccccc5c5ccccc53)c3ccc5c(c6ccccc6n5-c5ccccc5)c3n4-c3ccccc3)n2)cc1 ; c1ccc(-c2nc(-c3ccccc3)nc(-c3cc(-n4c5ccccc5c5ccccc54)cc4c5ccc6c(c7ccccc7n6-c6ccccc6)c5n(-c5ccccc5)c34)n2)cc1 ; c1ccc(-c2nc(-c3ccccc3)nc(-c3cc4c(cc3-c3ccc5c(c3)c3ccccc3n5-c3ccccc3)c3ccccc3n4-c3ccccc3)n2)cc1]

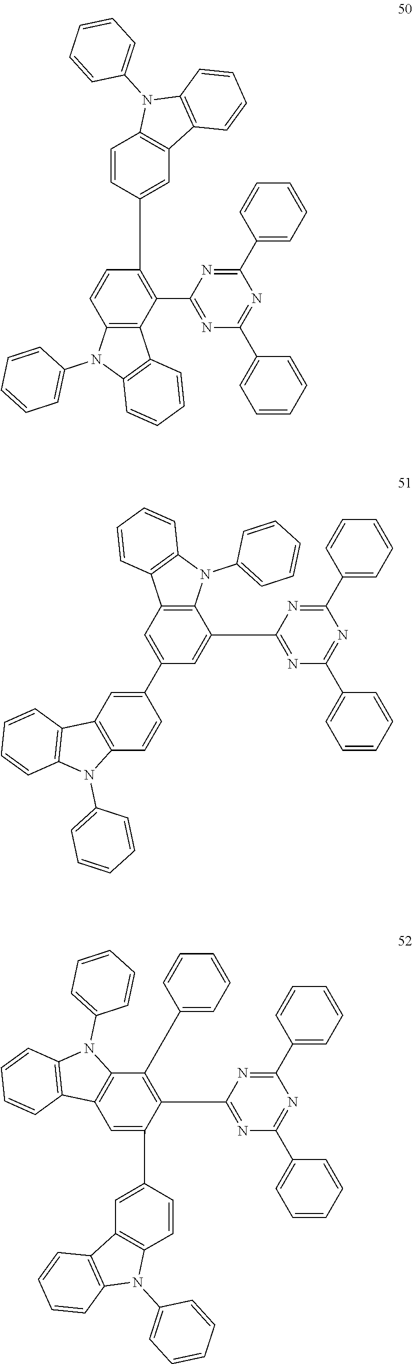 [SMILES: c1ccc(-c2nc(-c3ccccc3)nc(-c3c(-c4ccc5c(c4)c4ccccc4n5-c4ccccc4)ccc4c3c3ccccc3n4-c3ccccc3)n2)cc1 ; c1ccc(-c2nc(-c3ccccc3)nc(-c3cc(-c4ccc5c(c4)c4ccccc4n5-c4ccccc4)cc4c5ccccc5n(-c5ccccc5)c34)n2)cc1 ; c1ccc(-c2nc(-c3ccccc3)nc(-c3c(-c4ccc5c(c4)c4ccccc4n5-c4ccccc4)cc4c5ccccc5n(-c5ccccc5)c4c3-c3ccccc3)n2)cc1]

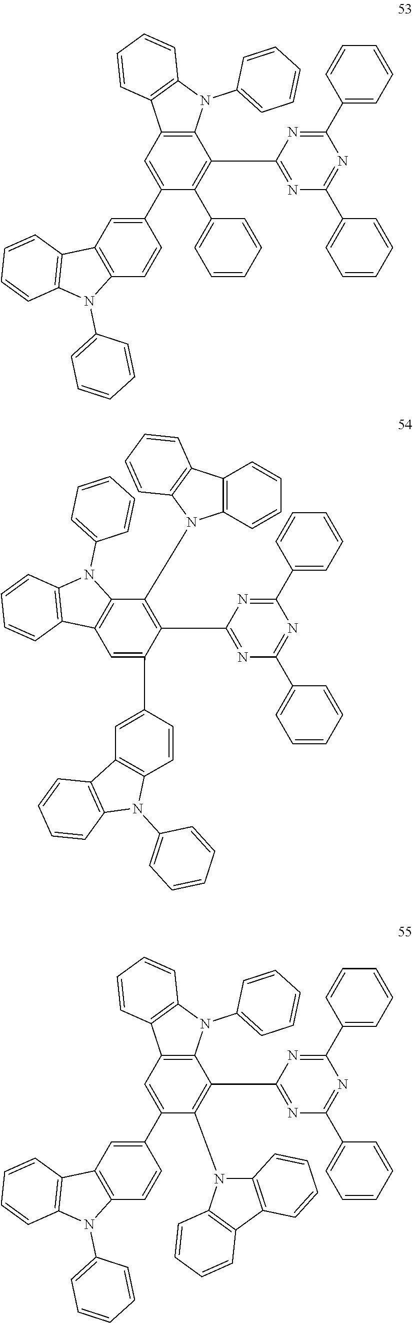 [SMILES: c1ccc(-c2nc(-c3ccccc3)nc(-c3c(-c4ccccc4)c(-c4ccc5c(c4)c4ccccc4n5-c4ccccc4)cc4c5ccccc5n(-c5ccccc5)c34)n2)cc1 ; c1ccc(-c2nc(-c3ccccc3)nc(-c3c(-c4ccc5c(c4)c4ccccc4n5-c4ccccc4)cc4c5ccccc5n(-c5ccccc5)c4c3-n3c4ccccc4c4ccccc43)n2)cc1 ; c1ccc(-c2nc(-c3ccccc3)nc(-c3c(-n4c5ccccc5c5ccccc54)c(-c4ccc5c(c4)c4ccccc4n5-c4ccccc4)cc4c5ccccc5n(-c5ccccc5)c34)n2)cc1]

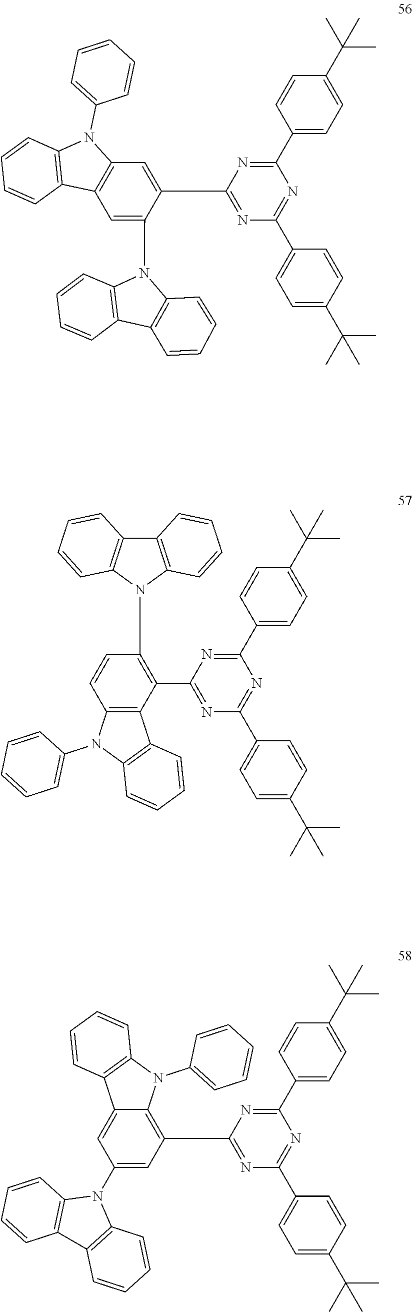 [SMILES: CC(C)(C)c1ccc(-c2nc(-c3ccc(C(C)(C)C)cc3)nc(-c3cc4c(cc3-n3c5ccccc5c5ccccc53)c3ccccc3n4-c3ccccc3)n2)cc1 ; CC(C)(C)c1ccc(-c2nc(-c3ccc(C(C)(C)C)cc3)nc(-c3c(-n4c5ccccc5c5ccccc54)ccc4c3c3ccccc3n4-c3ccccc3)n2)cc1 ; CC(C)(C)c1ccc(-c2nc(-c3ccc(C(C)(C)C)cc3)nc(-c3cc(-n4c5ccccc5c5ccccc54)cc4c5ccccc5n(-c5ccccc5)c34)n2)cc1]

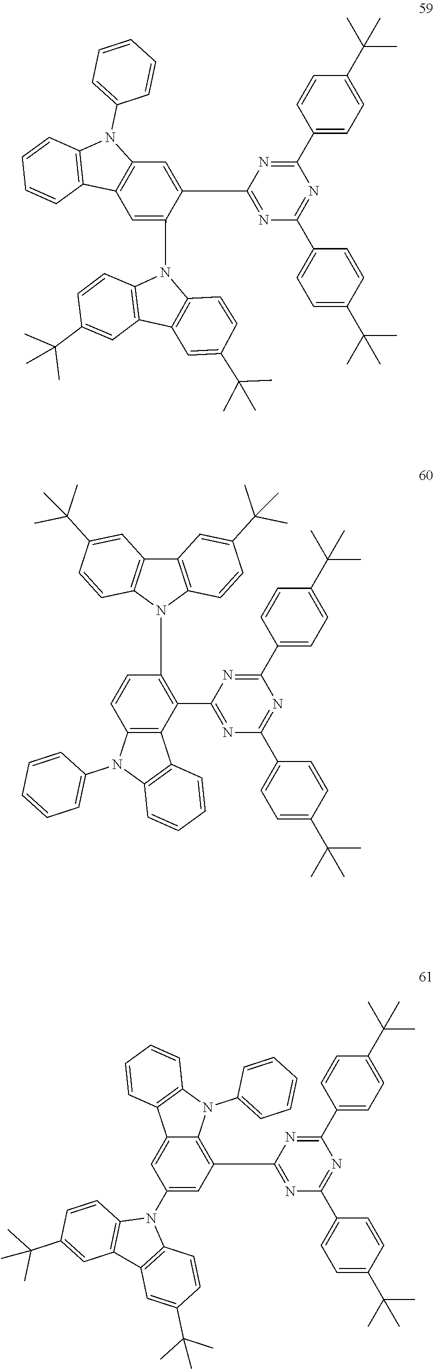

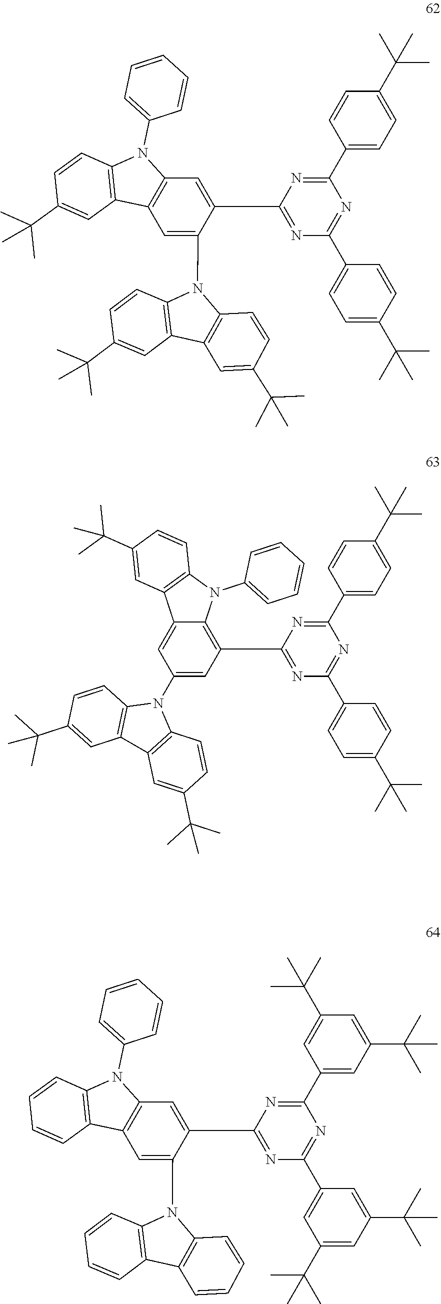

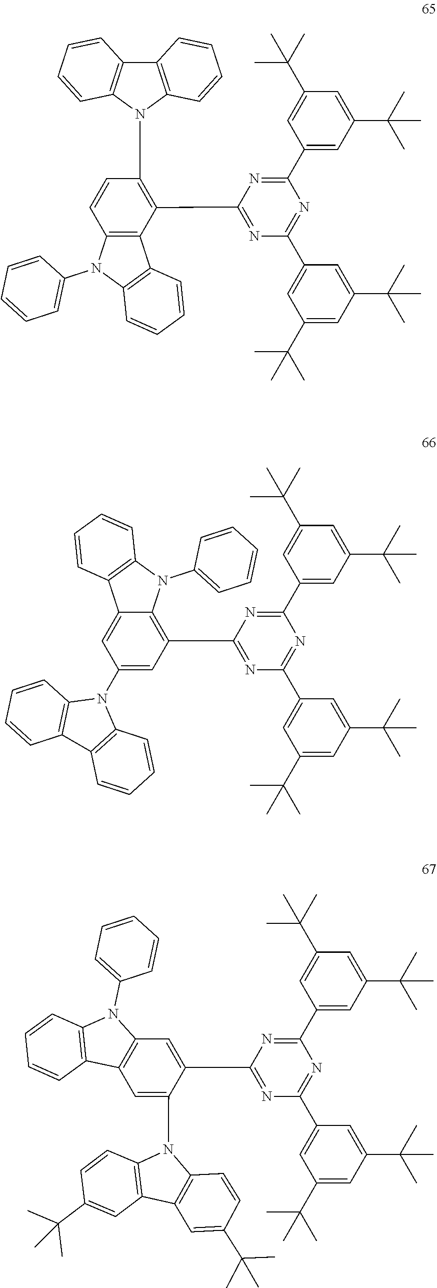 [SMILES: CC(C)(C)c1cc(-c2nc(-c3cc(C(C)(C)C)cc(C(C)(C)C)c3)nc(-c3c(-n4c5ccccc5c5ccccc54)ccc4c3c3ccccc3n4-c3ccccc3)n2)cc(C(C)(C)C)c1 ; CC(C)(C)c1cc(-c2nc(-c3cc(C(C)(C)C)cc(C(C)(C)C)c3)nc(-c3cc(-n4c5ccccc5c5ccccc54)cc4c5ccccc5n(-c5ccccc5)c34)n2)cc(C(C)(C)C)c1 ; CC(C)(C)c1cc(-c2nc(-c3cc(C(C)(C)C)cc(C(C)(C)C)c3)nc(-c3cc4c(cc3-n3c5ccc(C(C)(C)C)cc5c5cc(C(C)(C)C)ccc53)c3ccccc3n4-c3ccccc3)n2)cc(C(C)(C)C)c1]

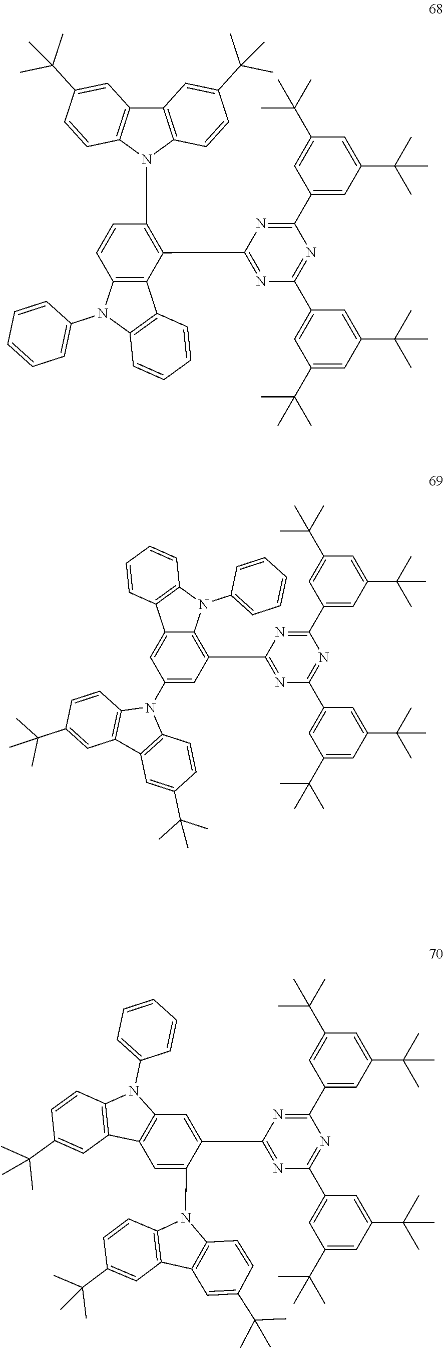

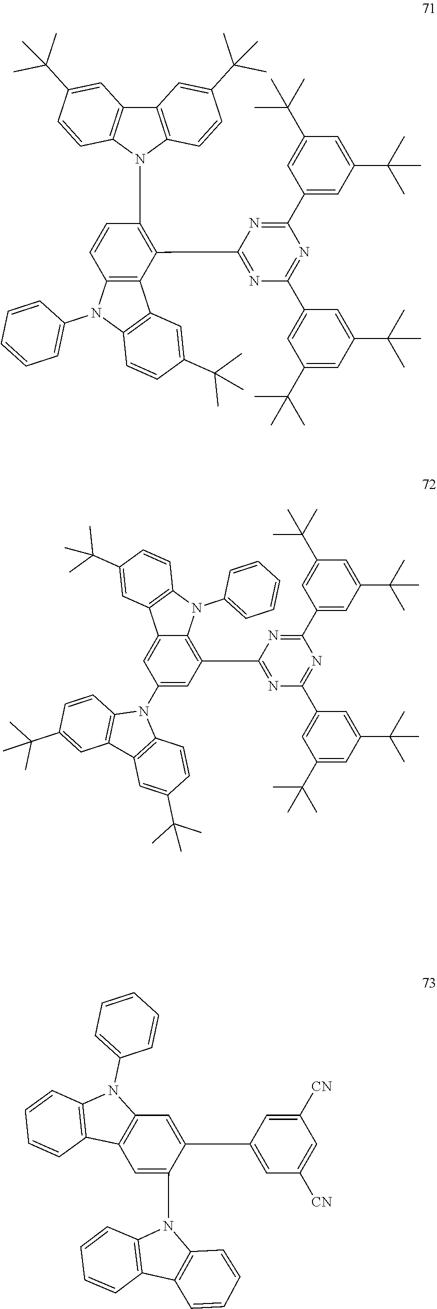

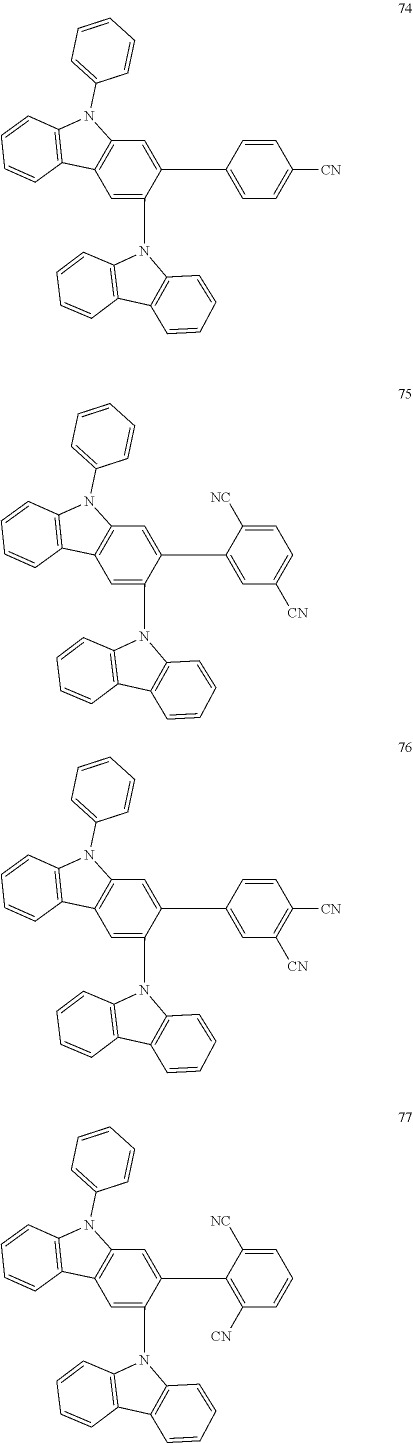

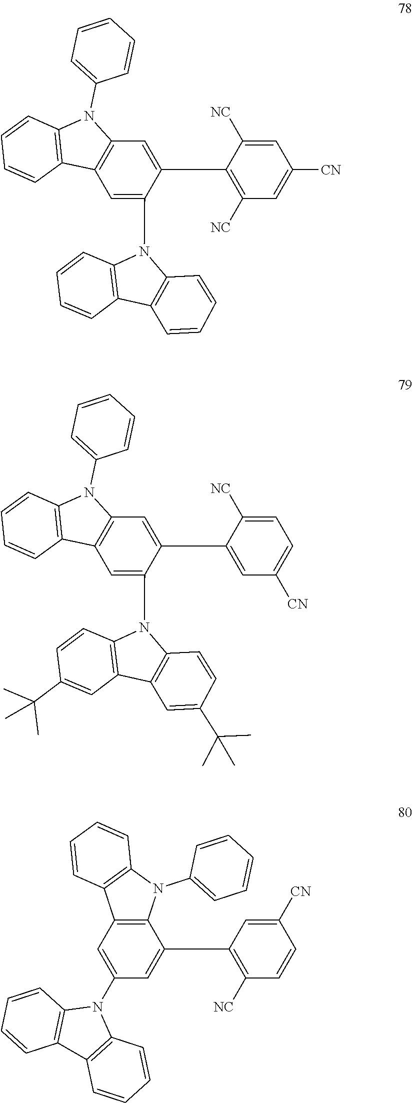 [SMILES: N#Cc1cc(C#N)c(-c2cc3c(cc2-n2c4ccccc4c4ccccc42)c2ccccc2n3-c2ccccc2)c(C#N)c1 ; CC(C)(C)c1ccc2c(c1)c1cc(C(C)(C)C)ccc1n2-c1cc2c3ccccc3n(-c3ccccc3)c2cc1-c1cc(C#N)ccc1C#N ; N#Cc1ccc(C#N)c(-c2cc(-n3c4ccccc4c4ccccc43)cc3c4ccccc4n(-c4ccccc4)c23)c1]

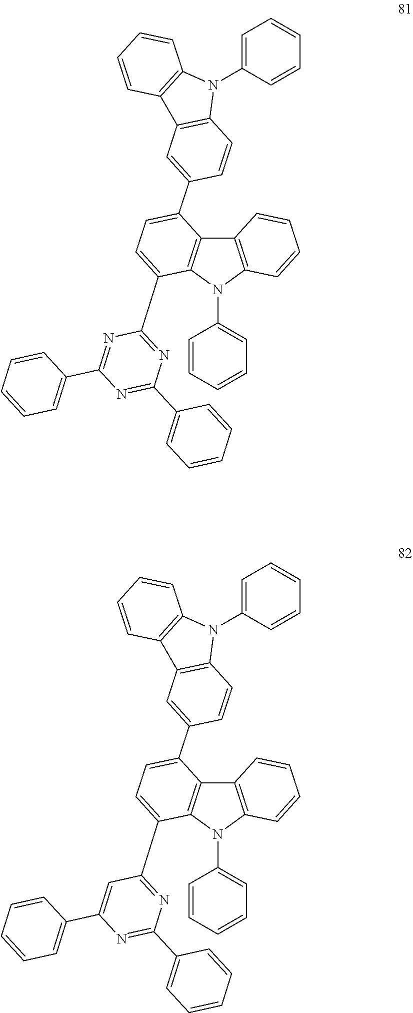 [SMILES: c1ccc(-c2nc(-c3ccccc3)nc(-c3ccc(-c4ccc5c(c4)c4ccccc4n5-c4ccccc4)c4c5ccccc5n(-c5ccccc5)c34)n2)cc1 ; c1ccc(-c2cc(-c3ccc(-c4ccc5c(c4)c4ccccc4n5-c4ccccc4)c4c5ccccc5n(-c5ccccc5)c34)nc(-c3ccccc3)n2)cc1]

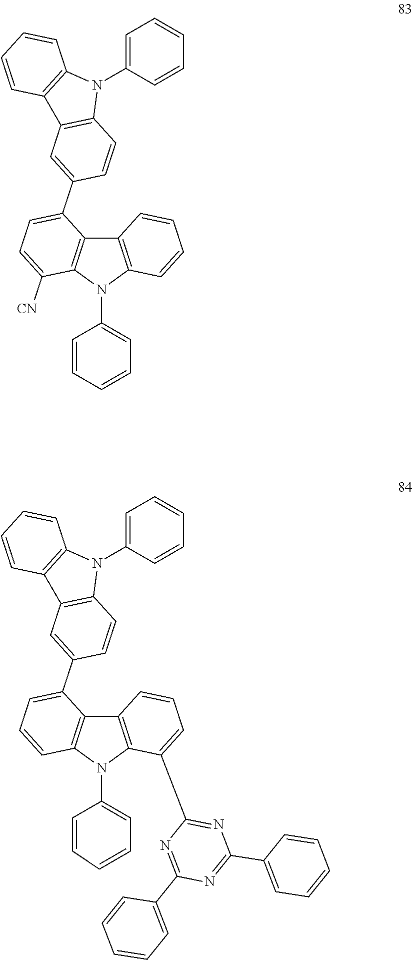

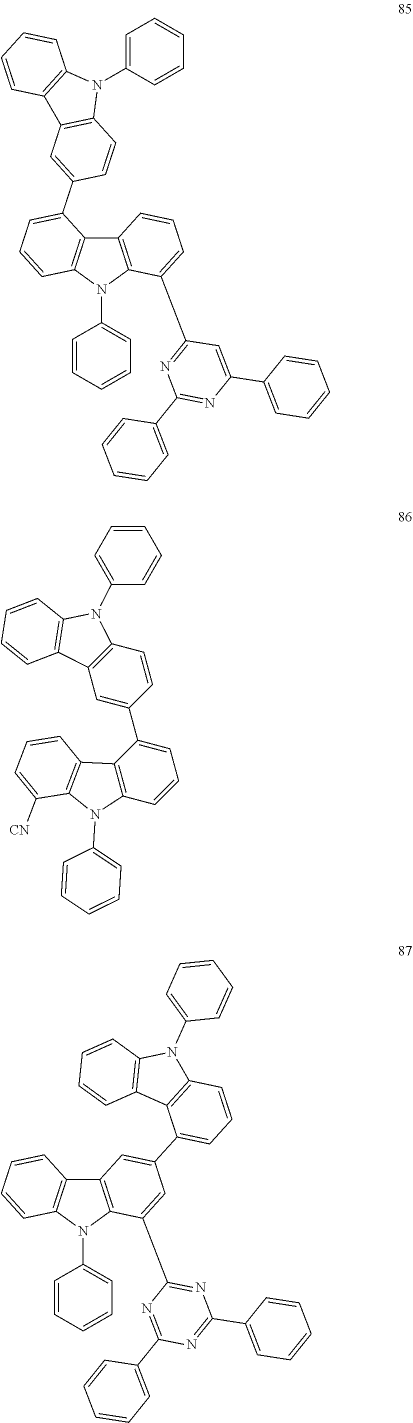

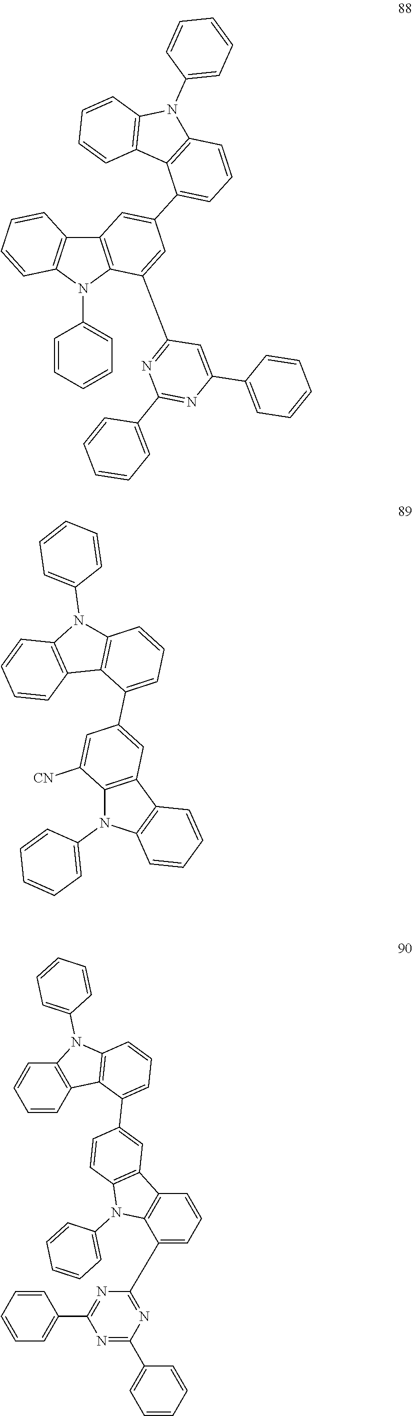

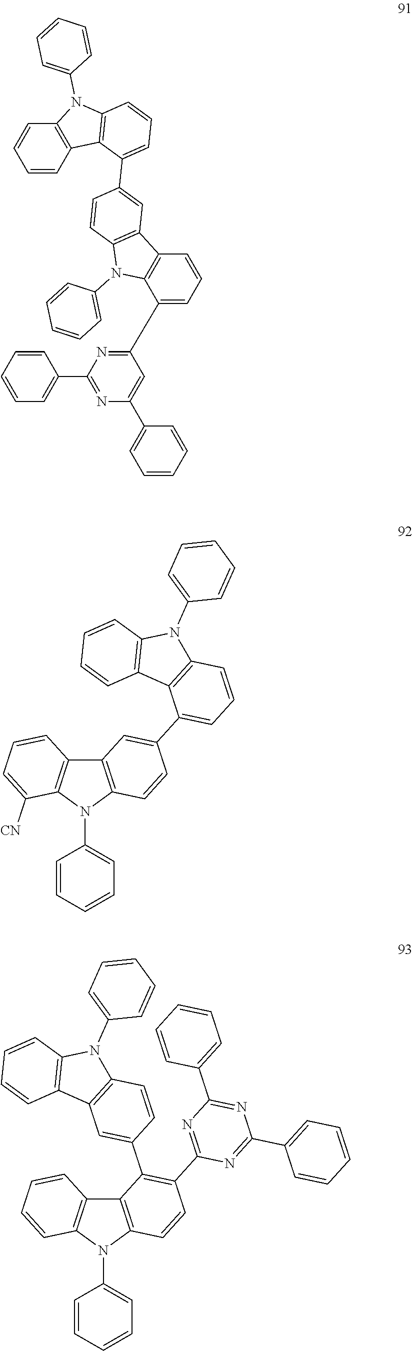

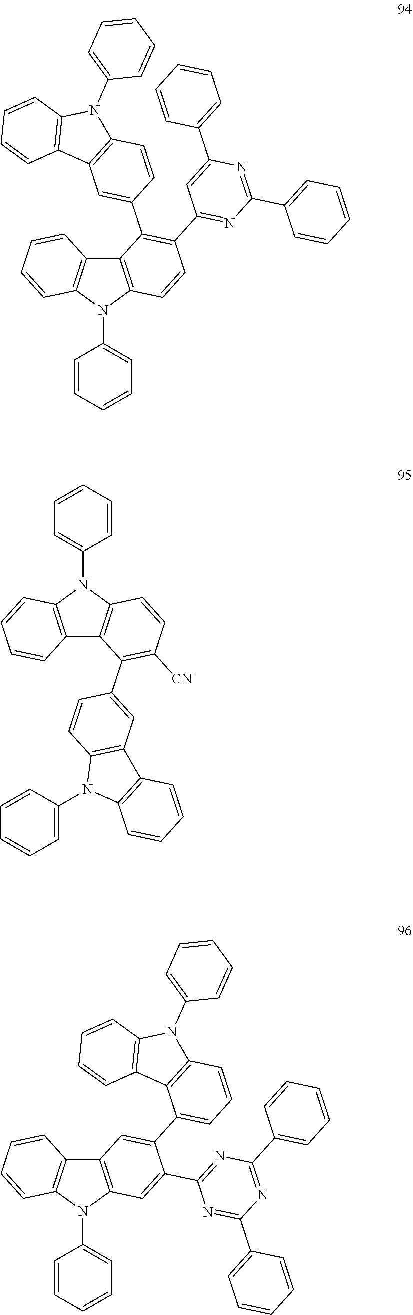 [SMILES: c1ccc(-c2cc(-c3ccc4c(c3-c3ccc5c(c3)c3ccccc3n5-c3ccccc3)c3ccccc3n4-c3ccccc3)nc(-c3ccccc3)n2)cc1 ; N#Cc1ccc2c(c1-c1ccc3c(c1)c1ccccc1n3-c1ccccc1)c1ccccc1n2-c1ccccc1 ; c1ccc(-c2nc(-c3ccccc3)nc(-c3cc4c(cc3-c3cccc5c3c3ccccc3n5-c3ccccc3)c3ccccc3n4-c3ccccc3)n2)cc1]

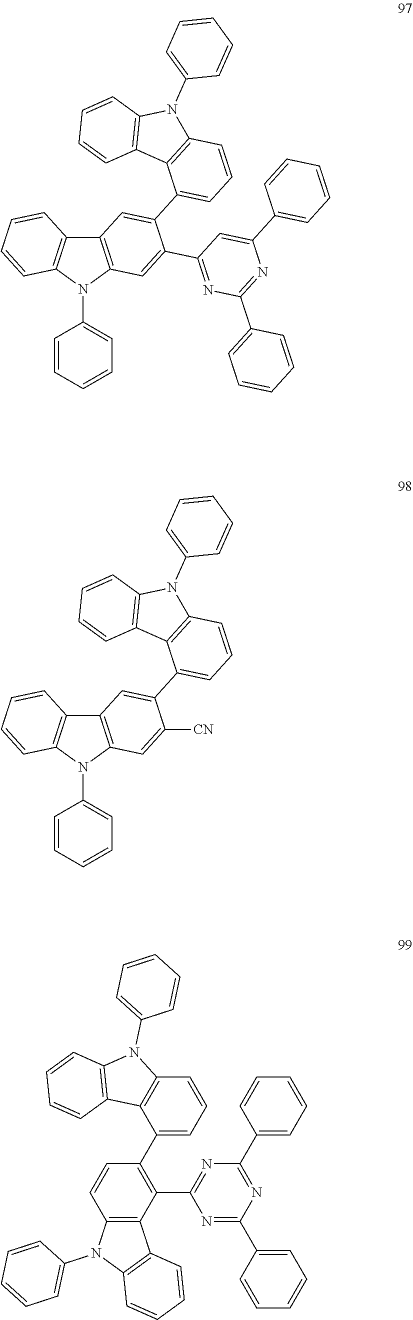 [SMILES: c1ccc(-c2cc(-c3cc4c(cc3-c3cccc5c3c3ccccc3n5-c3ccccc3)c3ccccc3n4-c3ccccc3)nc(-c3ccccc3)n2)cc1 ; N#Cc1cc2c(cc1-c1cccc3c1c1ccccc1n3-c1ccccc1)c1ccccc1n2-c1ccccc1 ; c1ccc(-c2nc(-c3ccccc3)nc(-c3c(-c4cccc5c4c4ccccc4n5-c4ccccc4)ccc4c3c3ccccc3n4-c3ccccc3)n2)cc1]

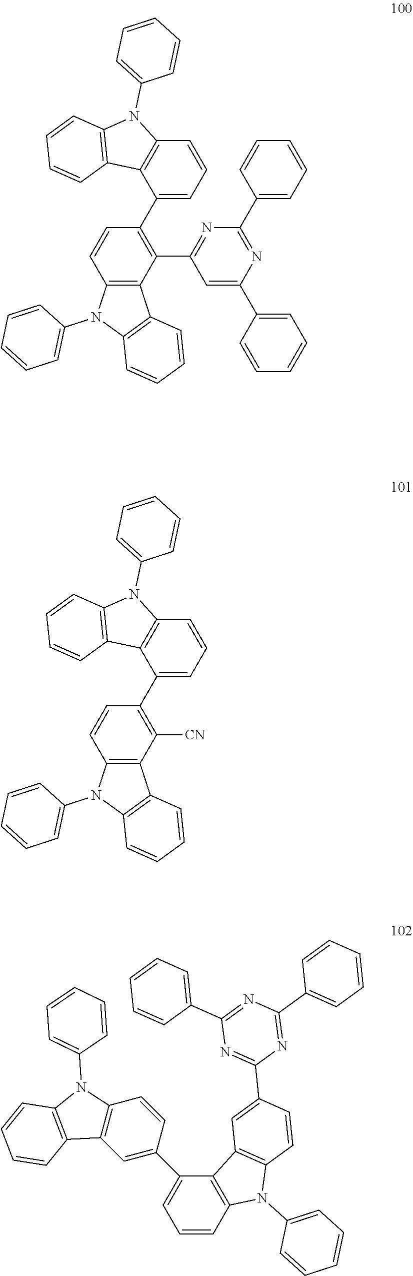 [SMILES: c1ccc(-c2cc(-c3c(-c4cccc5c4c4ccccc4n5-c4ccccc4)ccc4c3c3ccccc3n4-c3ccccc3)nc(-c3ccccc3)n2)cc1 ; N#Cc1c(-c2cccc3c2c2ccccc2n3-c2ccccc2)ccc2c1c1ccccc1n2-c1ccccc1 ; c1ccc(-c2nc(-c3ccccc3)nc(-c3ccc4c(c3)c3c(-c5ccc6c(c5)c5ccccc5n6-c5ccccc5)cccc3n4-c3ccccc3)n2)cc1]

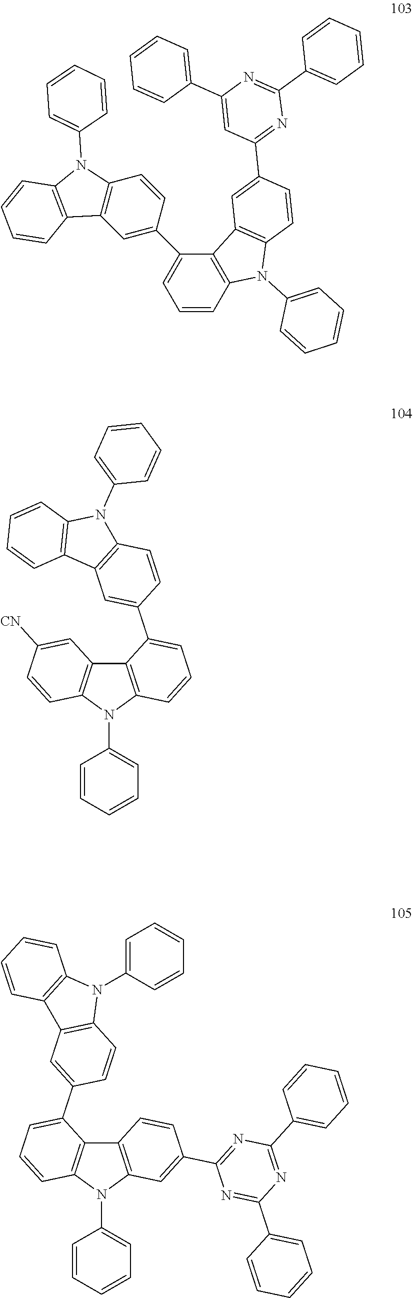

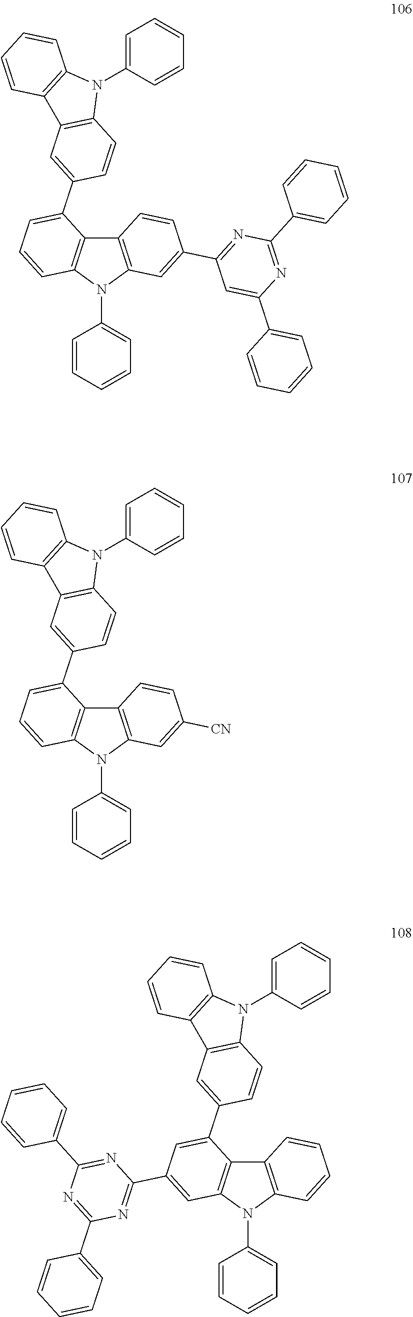 [SMILES: c1ccc(-c2cc(-c3ccc4c5c(-c6ccc7c(c6)c6ccccc6n7-c6ccccc6)cccc5n(-c5ccccc5)c4c3)nc(-c3ccccc3)n2)cc1 ; N#Cc1ccc2c3c(-c4ccc5c(c4)c4ccccc4n5-c4ccccc4)cccc3n(-c3ccccc3)c2c1 ; c1ccc(-c2nc(-c3ccccc3)nc(-c3cc(-c4ccc5c(c4)c4ccccc4n5-c4ccccc4)c4c5ccccc5n(-c5ccccc5)c4c3)n2)cc1]

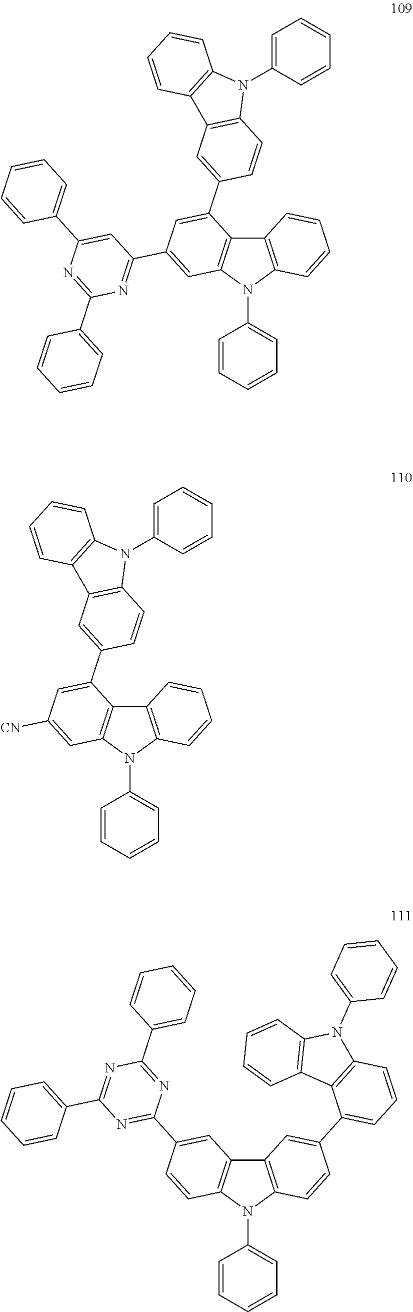

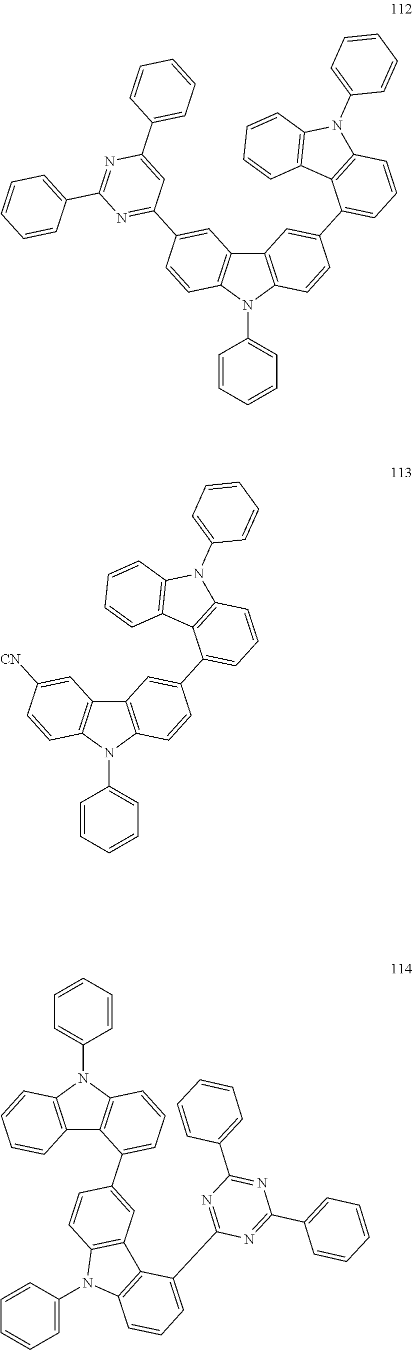

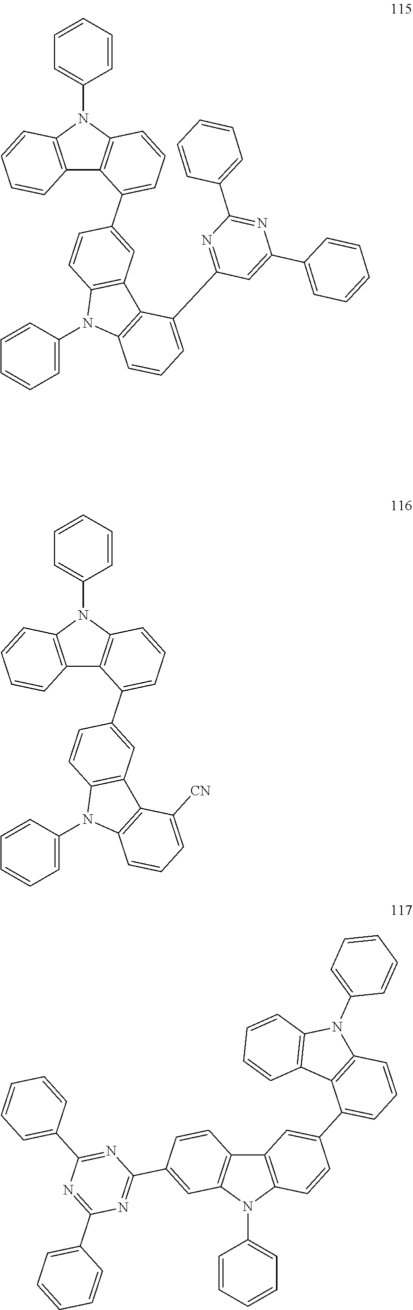 [SMILES: c1ccc(-c2cc(-c3cccc4c3c3cc(-c5cccc6c5c5ccccc5n6-c5ccccc5)ccc3n4-c3ccccc3)nc(-c3ccccc3)n2)cc1 ; N#Cc1cccc2c1c1cc(-c3cccc4c3c3ccccc3n4-c3ccccc3)ccc1n2-c1ccccc1 ; c1ccc(-c2nc(-c3ccccc3)nc(-c3ccc4c5cc(-c6cccc7c6c6ccccc6n7-c6ccccc6)ccc5n(-c5ccccc5)c4c3)n2)cc1]

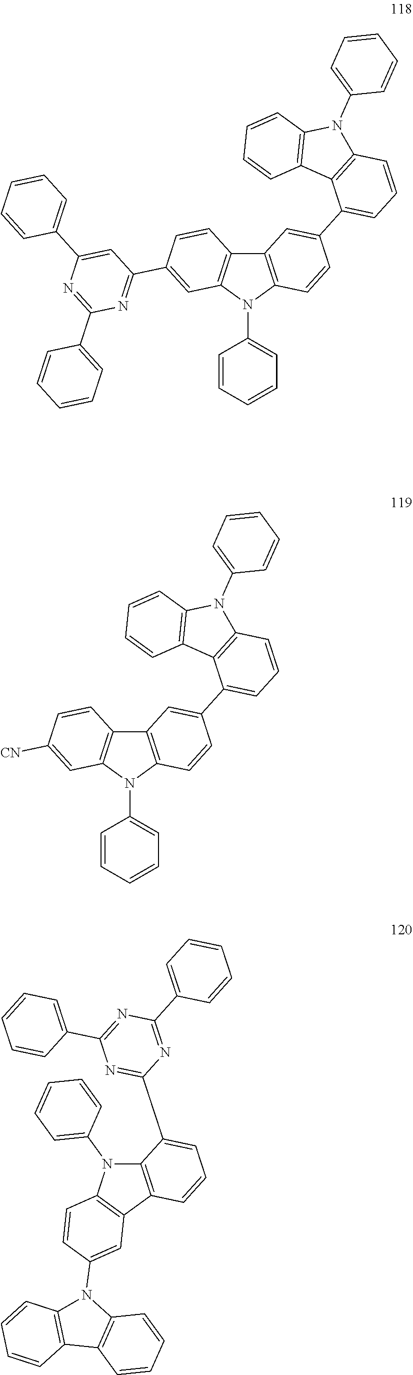

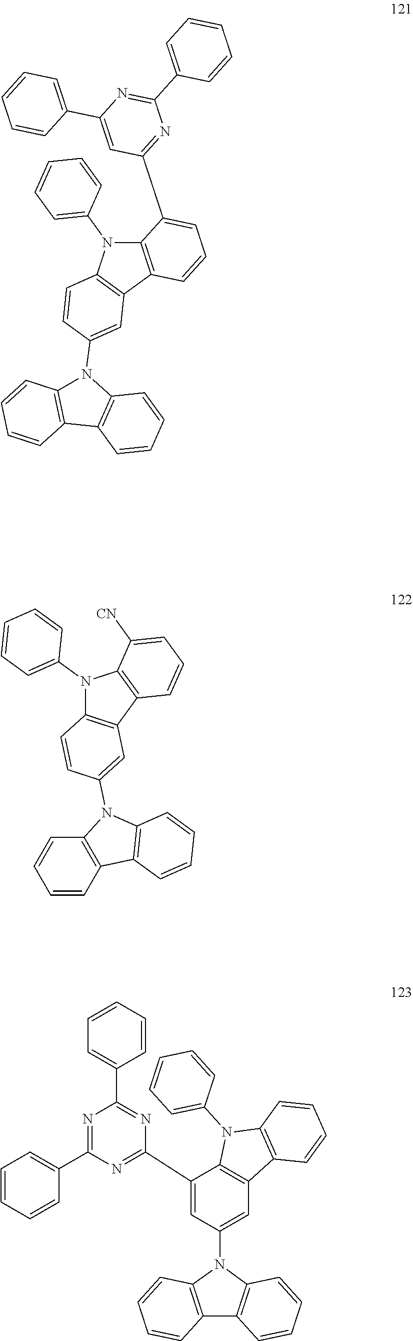

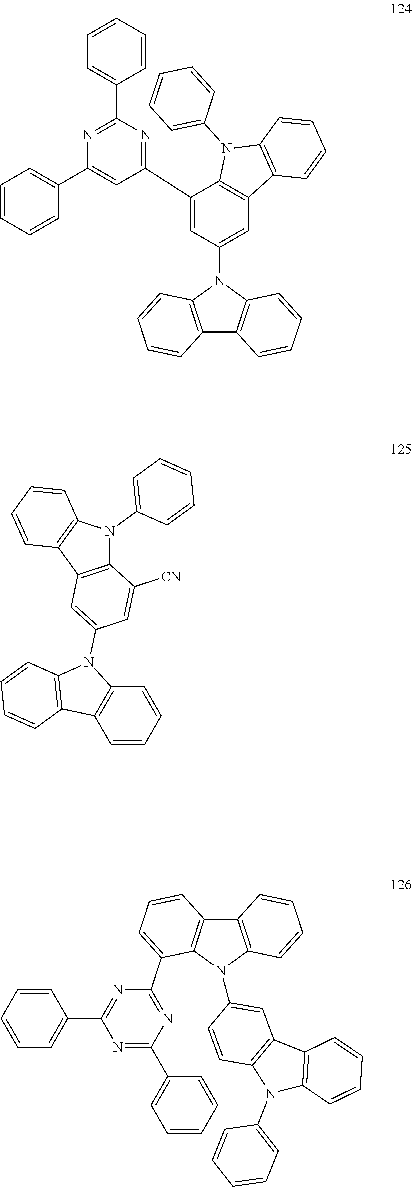 [SMILES: c1ccc(-c2cc(-c3cc(-n4c5ccccc5c5ccccc54)cc4c5ccccc5n(-c5ccccc5)c34)nc(-c3ccccc3)n2)cc1 ; N#Cc1cc(-n2c3ccccc3c3ccccc32)cc2c3ccccc3n(-c3ccccc3)c12 ; c1ccc(-c2nc(-c3ccccc3)nc(-c3cccc4c5ccccc5n(-c5ccc6c(c5)c5ccccc5n6-c5ccccc5)c34)n2)cc1]

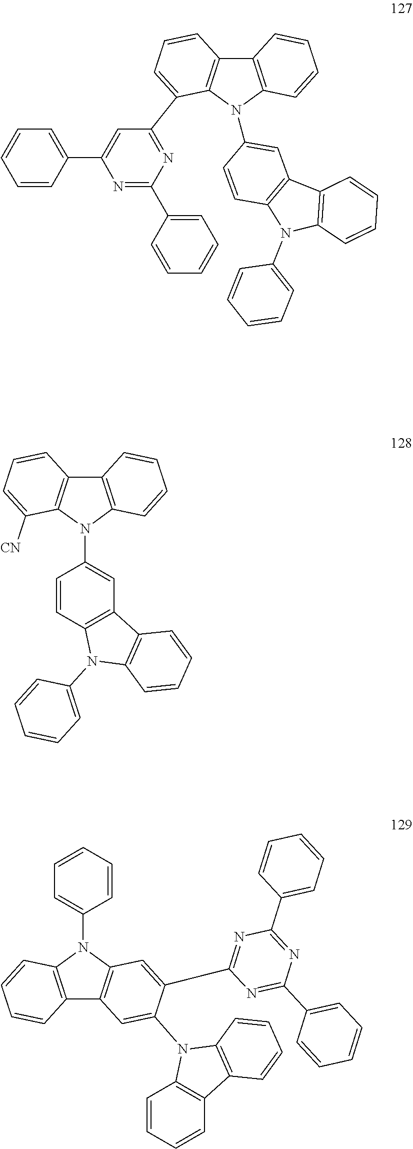

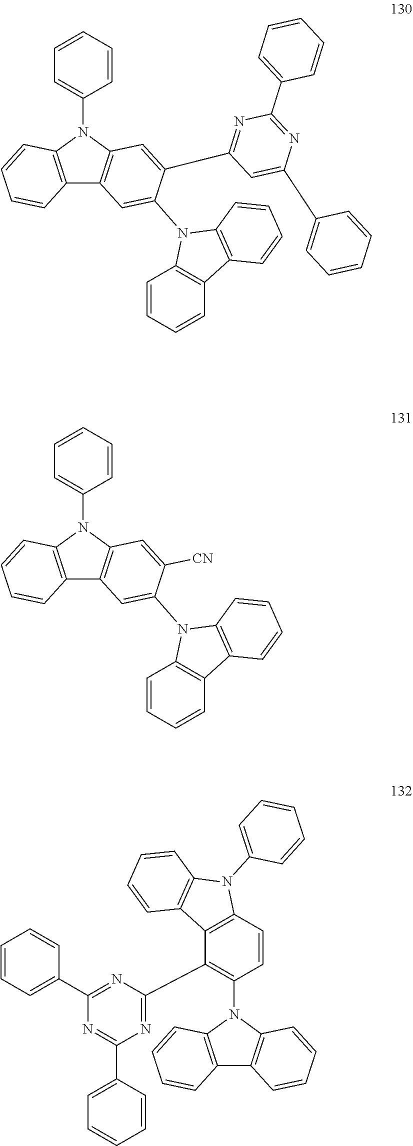 [SMILES: c1ccc(-c2cc(-c3cc4c(cc3-n3c5ccccc5c5ccccc53)c3ccccc3n4-c3ccccc3)nc(-c3ccccc3)n2)cc1 ; N#Cc1cc2c(cc1-n1c3ccccc3c3ccccc31)c1ccccc1n2-c1ccccc1 ; c1ccc(-c2nc(-c3ccccc3)nc(-c3c(-n4c5ccccc5c5ccccc54)ccc4c3c3ccccc3n4-c3ccccc3)n2)cc1]

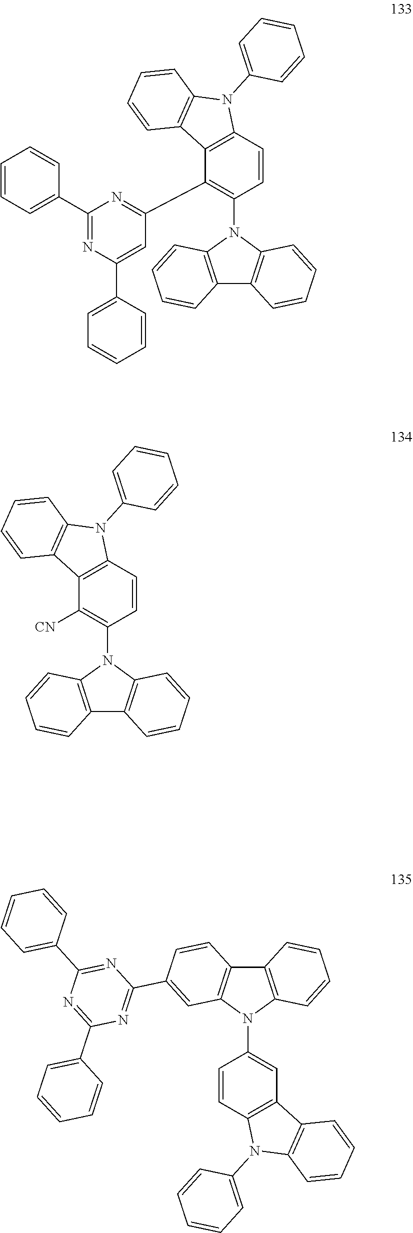

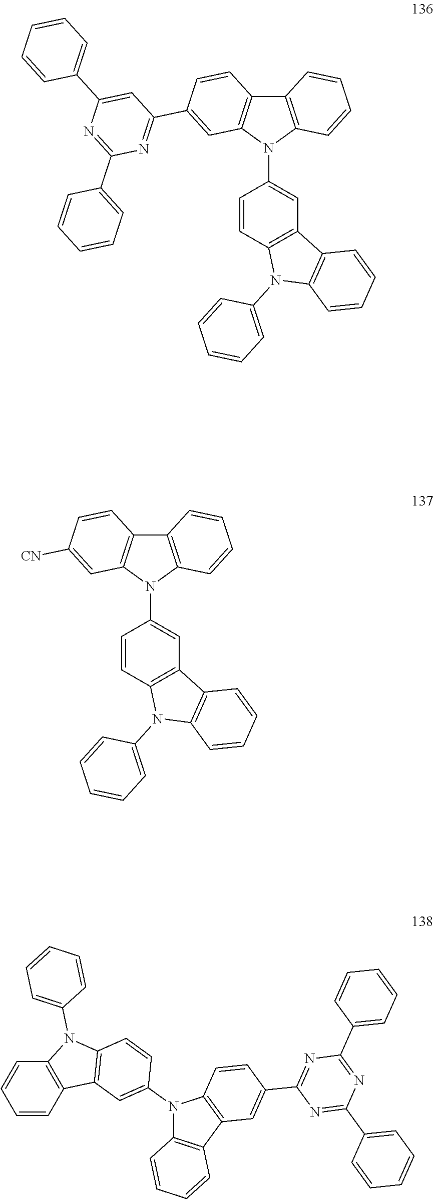

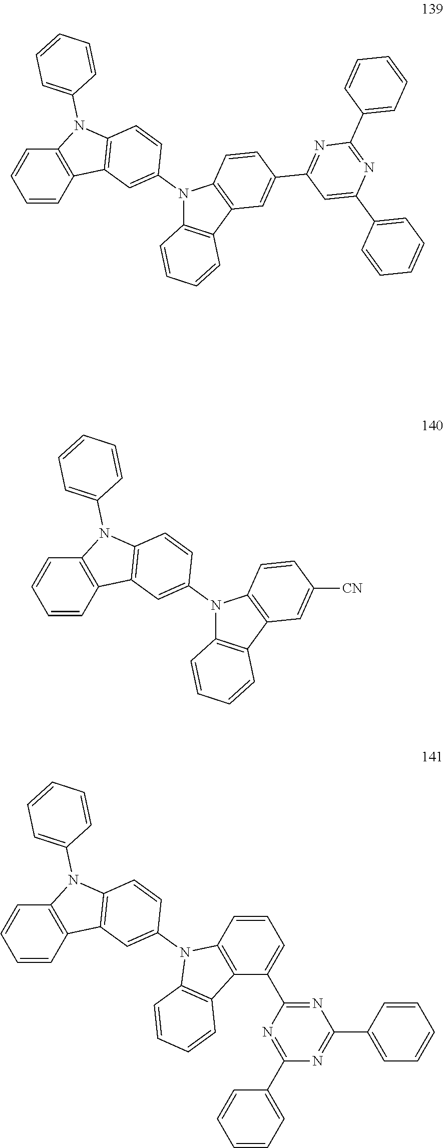 [SMILES: c1ccc(-c2cc(-c3ccc4c(c3)c3ccccc3n4-c3ccc4c(c3)c3ccccc3n4-c3ccccc3)nc(-c3ccccc3)n2)cc1 ; N#Cc1ccc2c(c1)c1ccccc1n2-c1ccc2c(c1)c1ccccc1n2-c1ccccc1 ; c1ccc(-c2nc(-c3ccccc3)nc(-c3cccc4c3c3ccccc3n4-c3ccc4c(c3)c3ccccc3n4-c3ccccc3)n2)cc1]

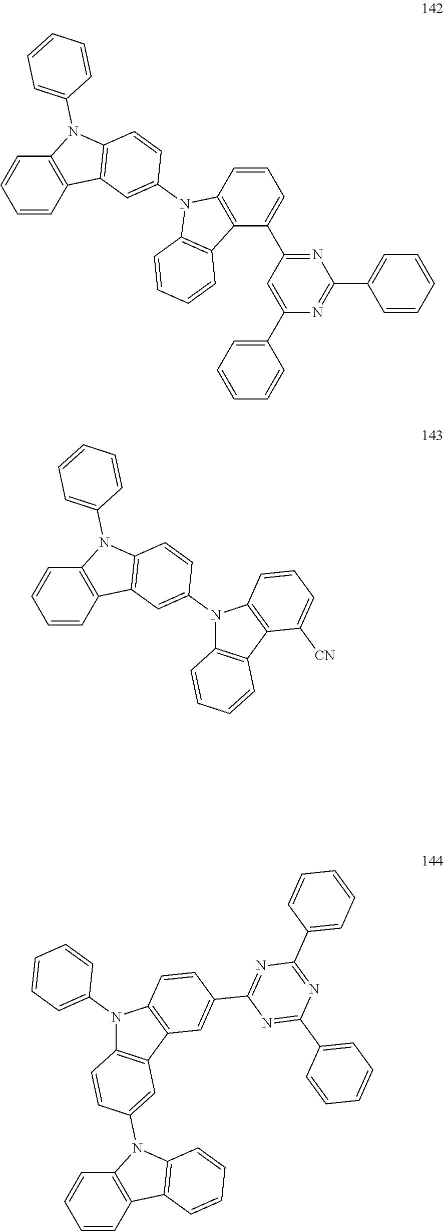 [SMILES: c1ccc(-c2cc(-c3cccc4c3c3ccccc3n4-c3ccc4c(c3)c3ccccc3n4-c3ccccc3)nc(-c3ccccc3)n2)cc1 ; N#Cc1cccc2c1c1ccccc1n2-c1ccc2c(c1)c1ccccc1n2-c1ccccc1 ; c1ccc(-c2nc(-c3ccccc3)nc(-c3ccc4c(c3)c3cc(-n5c6ccccc6c6ccccc65)ccc3n4-c3ccccc3)n2)cc1]

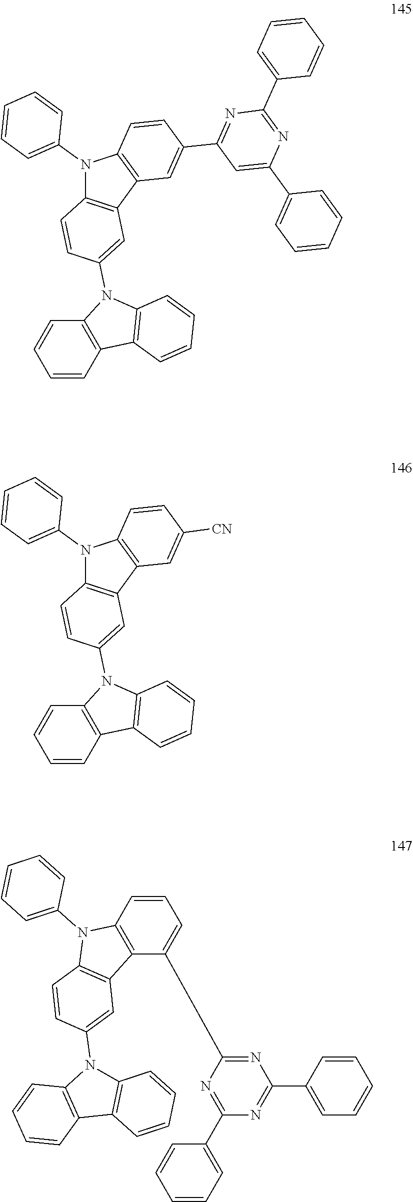 [SMILES: c1ccc(-c2cc(-c3ccc4c(c3)c3cc(-n5c6ccccc6c6ccccc65)ccc3n4-c3ccccc3)nc(-c3ccccc3)n2)cc1 ; N#Cc1ccc2c(c1)c1cc(-n3c4ccccc4c4ccccc43)ccc1n2-c1ccccc1 ; c1ccc(-c2nc(-c3ccccc3)nc(-c3cccc4c3c3cc(-n5c6ccccc6c6ccccc65)ccc3n4-c3ccccc3)n2)cc1]

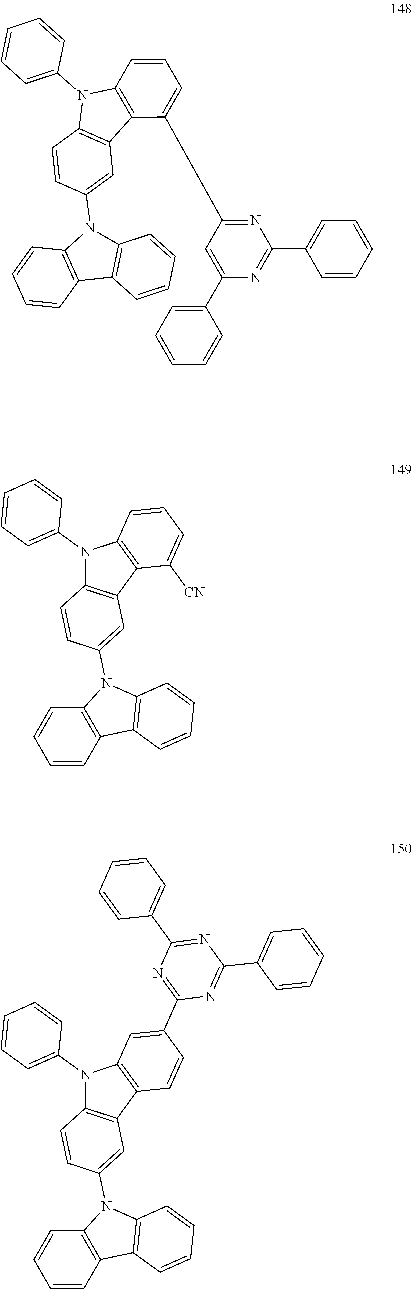 [SMILES: c1ccc(-c2cc(-c3cccc4c3c3cc(-n5c6ccccc6c6ccccc65)ccc3n4-c3ccccc3)nc(-c3ccccc3)n2)cc1 ; N#Cc1cccc2c1c1cc(-n3c4ccccc4c4ccccc43)ccc1n2-c1ccccc1 ; c1ccc(-c2nc(-c3ccccc3)nc(-c3ccc4c5cc(-n6c7ccccc7c7ccccc76)ccc5n(-c5ccccc5)c4c3)n2)cc1]

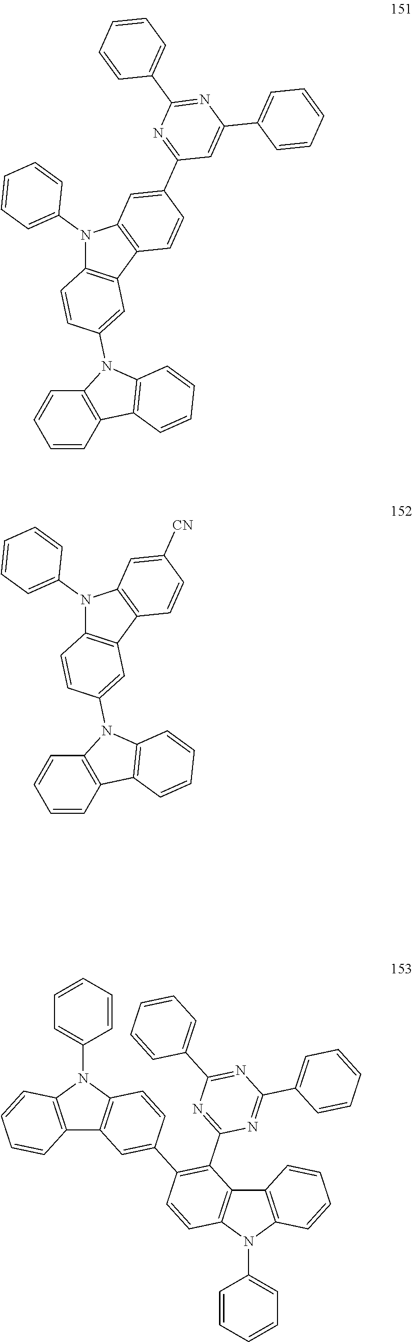 [SMILES: c1ccc(-c2cc(-c3ccc4c5cc(-n6c7ccccc7c7ccccc76)ccc5n(-c5ccccc5)c4c3)nc(-c3ccccc3)n2)cc1 ; N#Cc1ccc2c3cc(-n4c5ccccc5c5ccccc54)ccc3n(-c3ccccc3)c2c1 ; c1ccc(-c2nc(-c3ccccc3)nc(-c3c(-c4ccc5c(c4)c4ccccc4n5-c4ccccc4)ccc4c3c3ccccc3n4-c3ccccc3)n2)cc1]

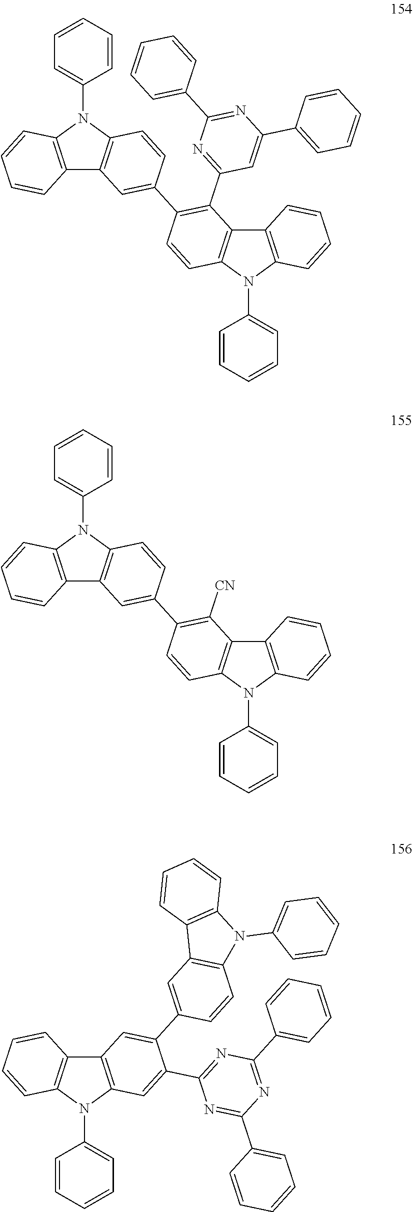

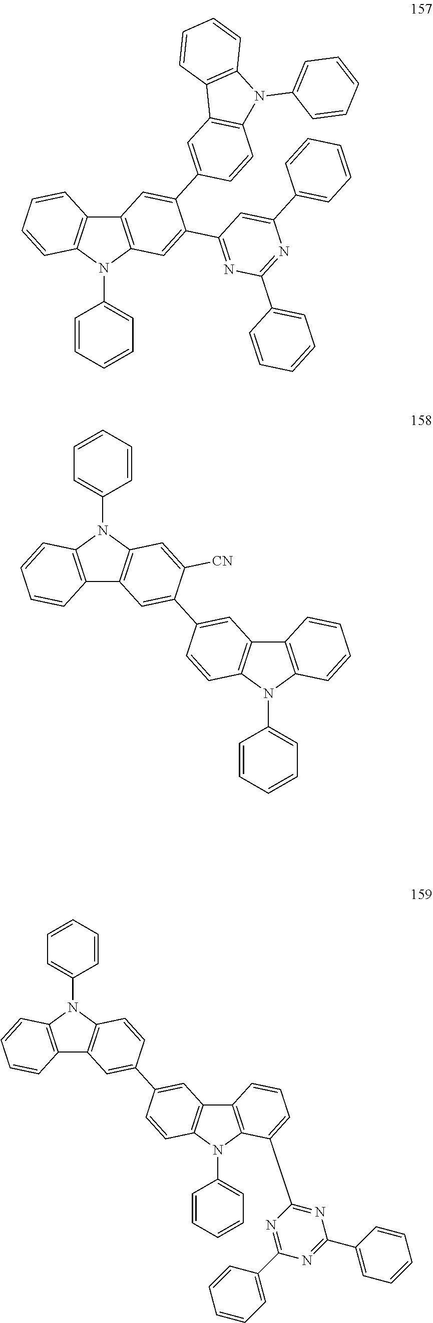

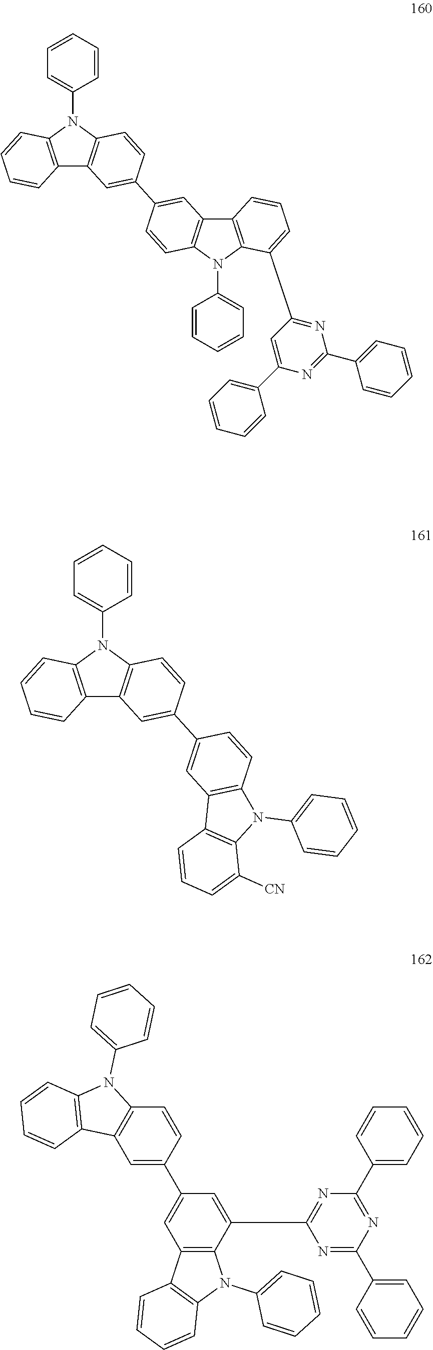 [SMILES: c1ccc(-c2cc(-c3cccc4c5cc(-c6ccc7c(c6)c6ccccc6n7-c6ccccc6)ccc5n(-c5ccccc5)c34)nc(-c3ccccc3)n2)cc1 ; N#Cc1cccc2c3cc(-c4ccc5c(c4)c4ccccc4n5-c4ccccc4)ccc3n(-c3ccccc3)c12 ; c1ccc(-c2nc(-c3ccccc3)nc(-c3cc(-c4ccc5c(c4)c4ccccc4n5-c4ccccc4)cc4c5ccccc5n(-c5ccccc5)c34)n2)cc1]

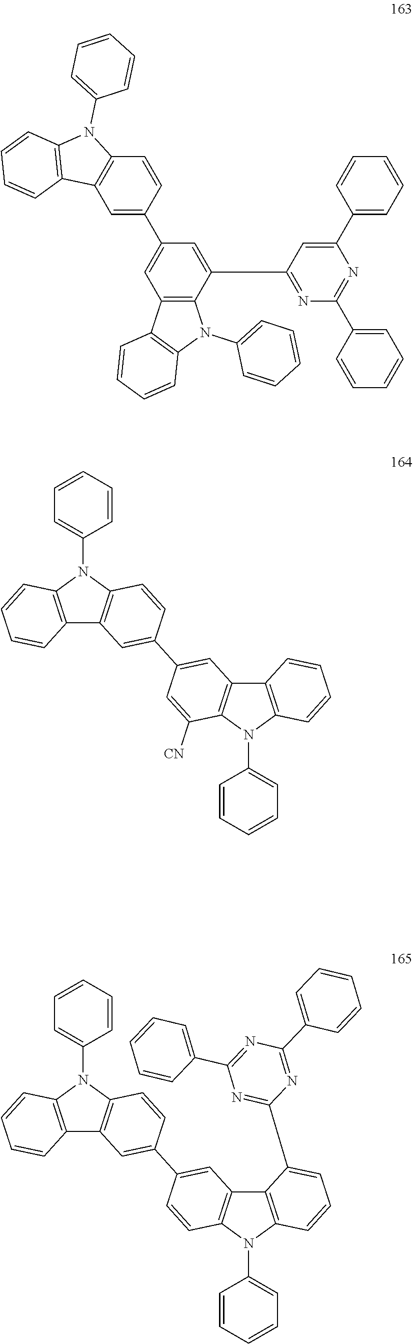

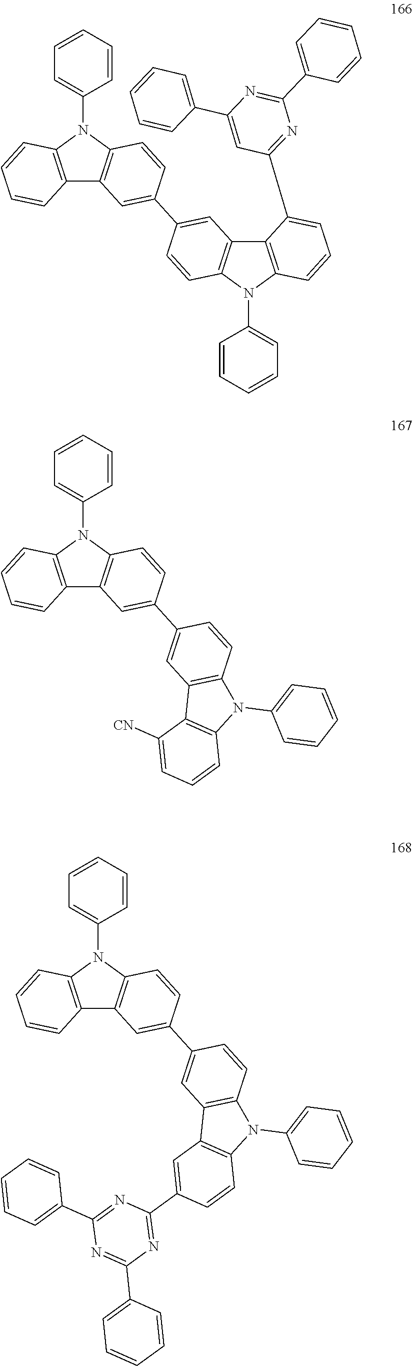

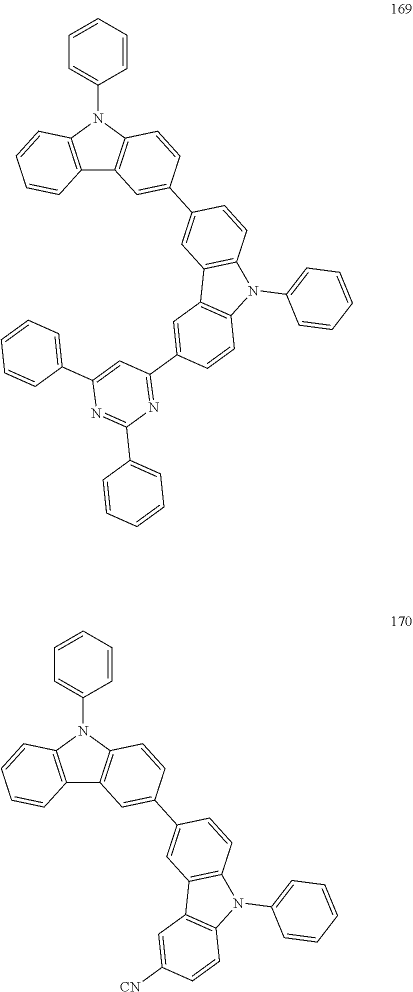

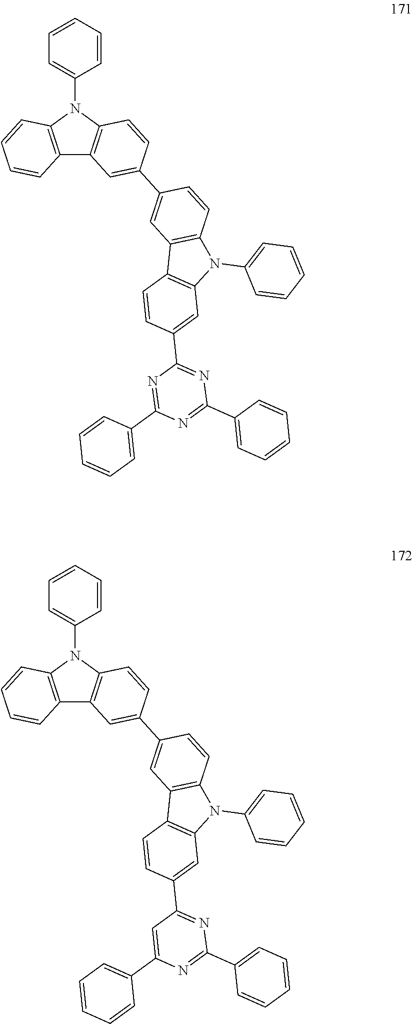 [SMILES: c1ccc(-c2nc(-c3ccccc3)nc(-c3ccc4c5cc(-c6ccc7c(c6)c6ccccc6n7-c6ccccc6)ccc5n(-c5ccccc5)c4c3)n2)cc1 ; c1ccc(-c2cc(-c3ccc4c5cc(-c6ccc7c(c6)c6ccccc6n7-c6ccccc6)ccc5n(-c5ccccc5)c4c3)nc(-c3ccccc3)n2)cc1]

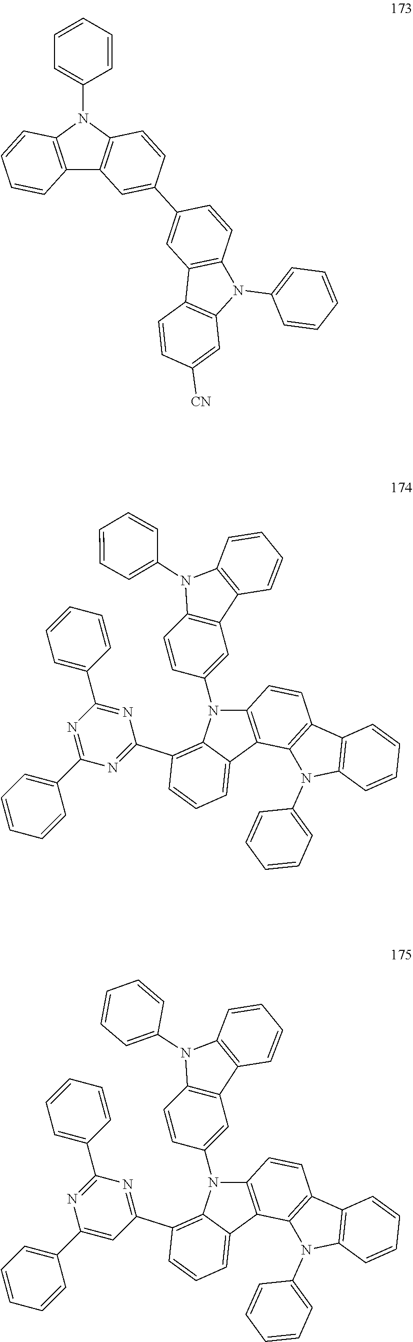 [SMILES: N#Cc1ccc2c3cc(-c4ccc5c(c4)c4ccccc4n5-c4ccccc4)ccc3n(-c3ccccc3)c2c1 ; c1ccc(-c2nc(-c3ccccc3)nc(-c3cccc4c5c(ccc6c7ccccc7n(-c7ccccc7)c65)n(-c5ccc6c(c5)c5ccccc5n6-c5ccccc5)c34)n2)cc1 ; c1ccc(-c2cc(-c3cccc4c5c(ccc6c7ccccc7n(-c7ccccc7)c65)n(-c5ccc6c(c5)c5ccccc5n6-c5ccccc5)c34)nc(-c3ccccc3)n2)cc1]

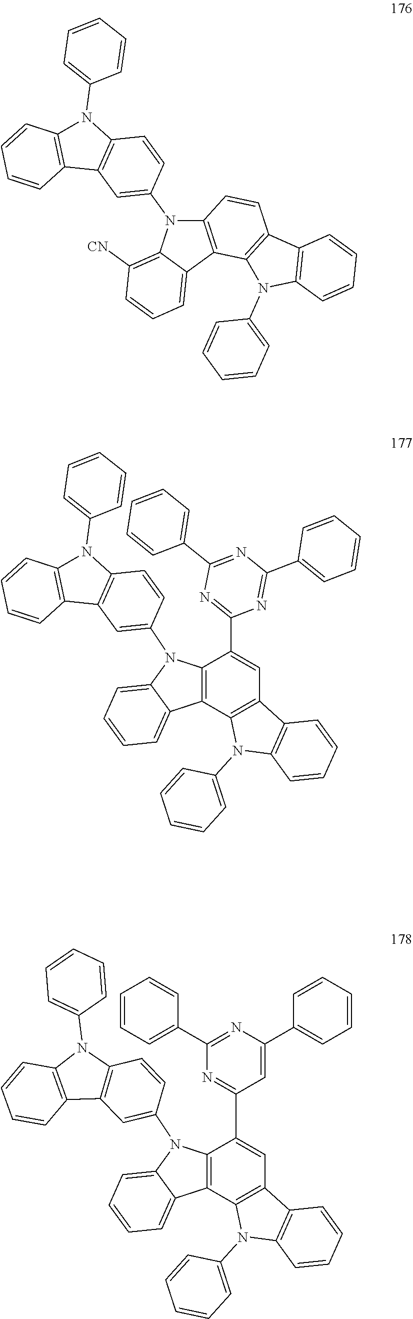

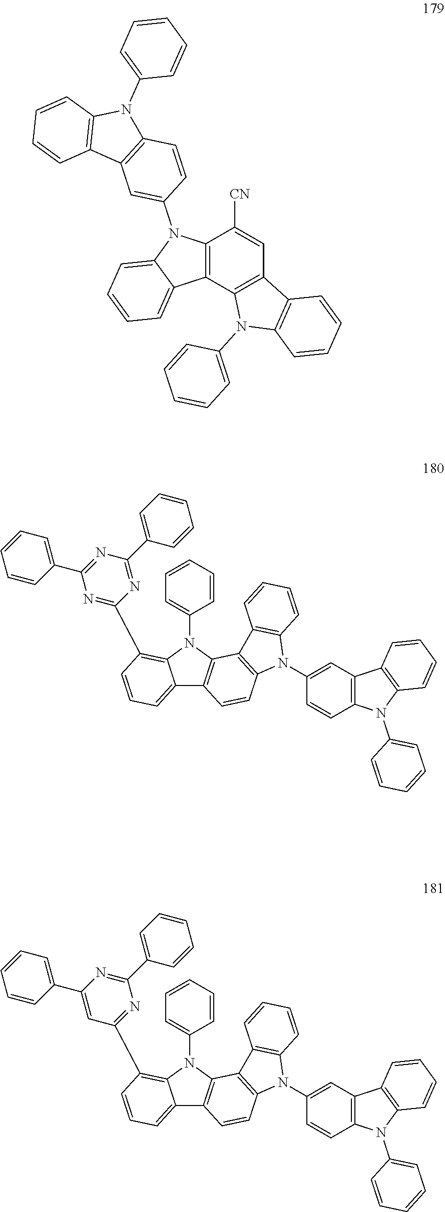 [SMILES: N#Cc1cc2c3ccccc3n(-c3ccccc3)c2c2c3ccccc3n(-c3ccc4c(c3)c3ccccc3n4-c3ccccc3)c12 ; c1ccc(-c2nc(-c3ccccc3)nc(-c3cccc4c5ccc6c(c7ccccc7n6-c6ccc7c(c6)c6ccccc6n7-c6ccccc6)c5n(-c5ccccc5)c34)n2)cc1 ; c1ccc(-c2cc(-c3cccc4c5ccc6c(c7ccccc7n6-c6ccc7c(c6)c6ccccc6n7-c6ccccc6)c5n(-c5ccccc5)c34)nc(-c3ccccc3)n2)cc1]

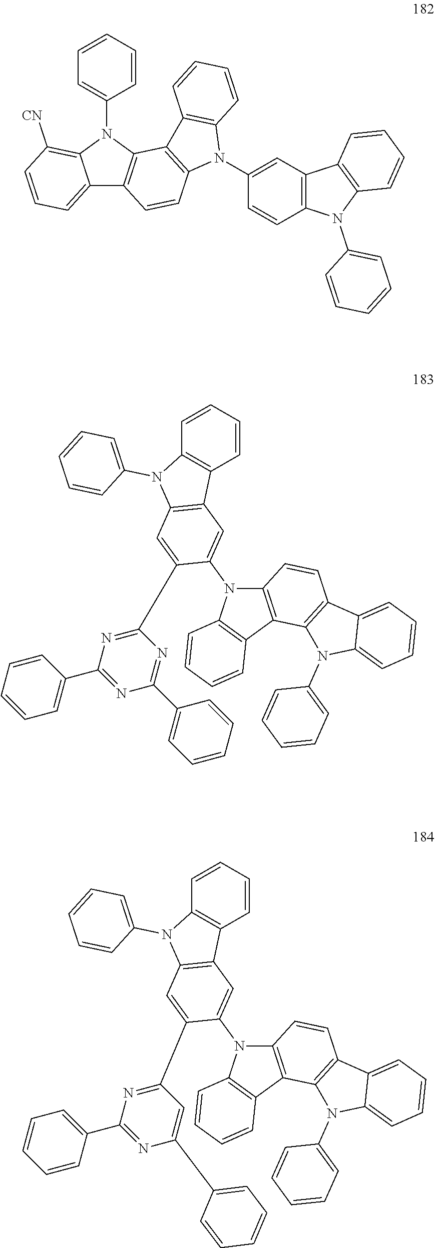

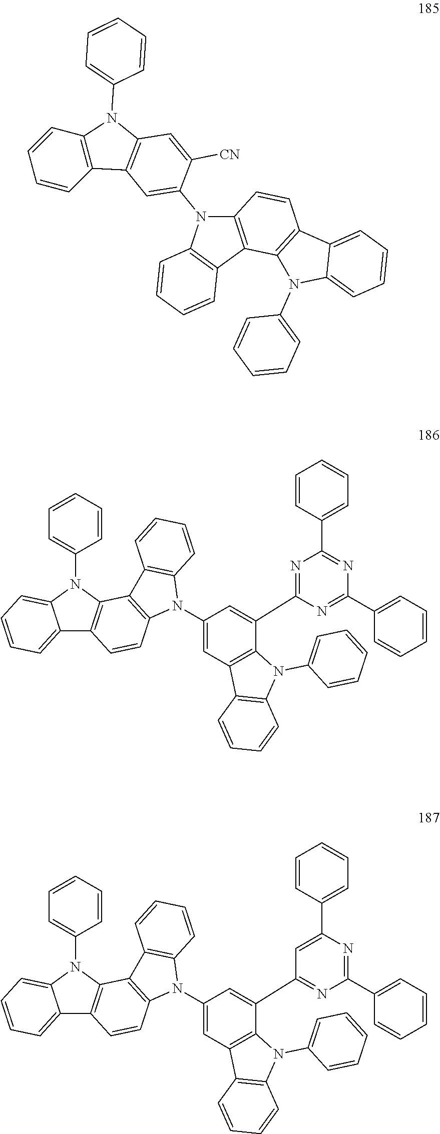 [SMILES: N#Cc1cc2c(cc1-n1c3ccccc3c3c1ccc1c4ccccc4n(-c4ccccc4)c13)c1ccccc1n2-c1ccccc1 ; c1ccc(-c2nc(-c3ccccc3)nc(-c3cc(-n4c5ccccc5c5c4ccc4c6ccccc6n(-c6ccccc6)c45)cc4c5ccccc5n(-c5ccccc5)c34)n2)cc1 ; c1ccc(-c2cc(-c3cc(-n4c5ccccc5c5c4ccc4c6ccccc6n(-c6ccccc6)c45)cc4c5ccccc5n(-c5ccccc5)c34)nc(-c3ccccc3)n2)cc1]

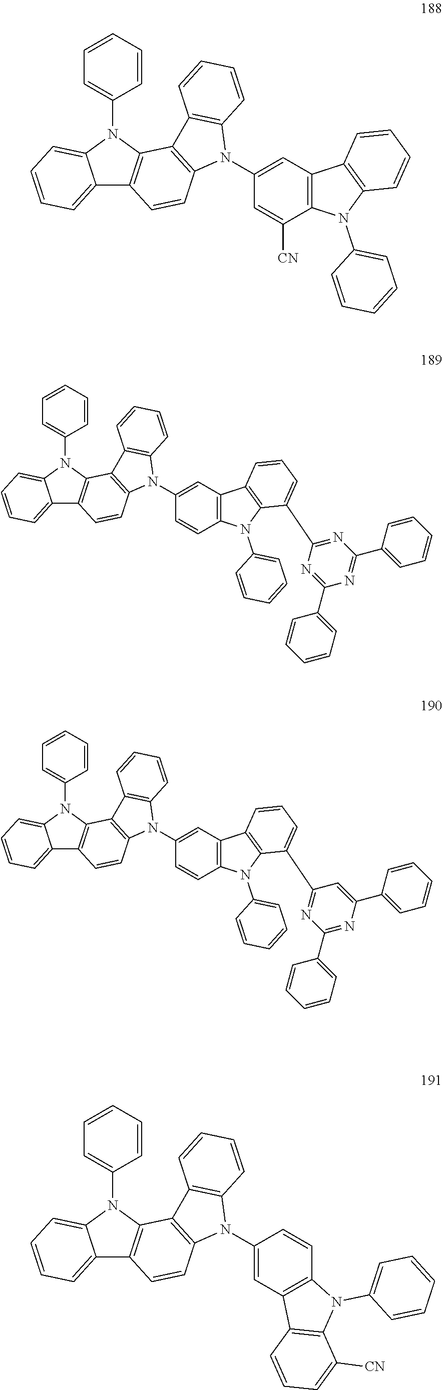 [SMILES: N#Cc1cc(-n2c3ccccc3c3c2ccc2c4ccccc4n(-c4ccccc4)c23)cc2c3ccccc3n(-c3ccccc3)c12 ; c1ccc(-c2nc(-c3ccccc3)nc(-c3cccc4c5cc(-n6c7ccccc7c7c6ccc6c8ccccc8n(-c8ccccc8)c67)ccc5n(-c5ccccc5)c34)n2)cc1 ; c1ccc(-c2cc(-c3cccc4c5cc(-n6c7ccccc7c7c6ccc6c8ccccc8n(-c8ccccc8)c67)ccc5n(-c5ccccc5)c34)nc(-c3ccccc3)n2)cc1 ; N#Cc1cccc2c3cc(-n4c5ccccc5c5c4ccc4c6ccccc6n(-c6ccccc6)c45)ccc3n(-c3ccccc3)c12]

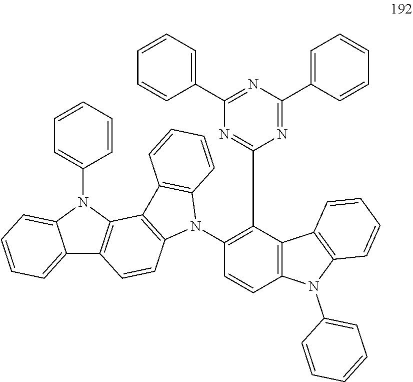 [SMILES: c1ccc(-c2nc(-c3ccccc3)nc(-c3c(-n4c5ccccc5c5c4ccc4c6ccccc6n(-c6ccccc6)c45)ccc4c3c3ccccc3n4-c3ccccc3)n2)cc1]

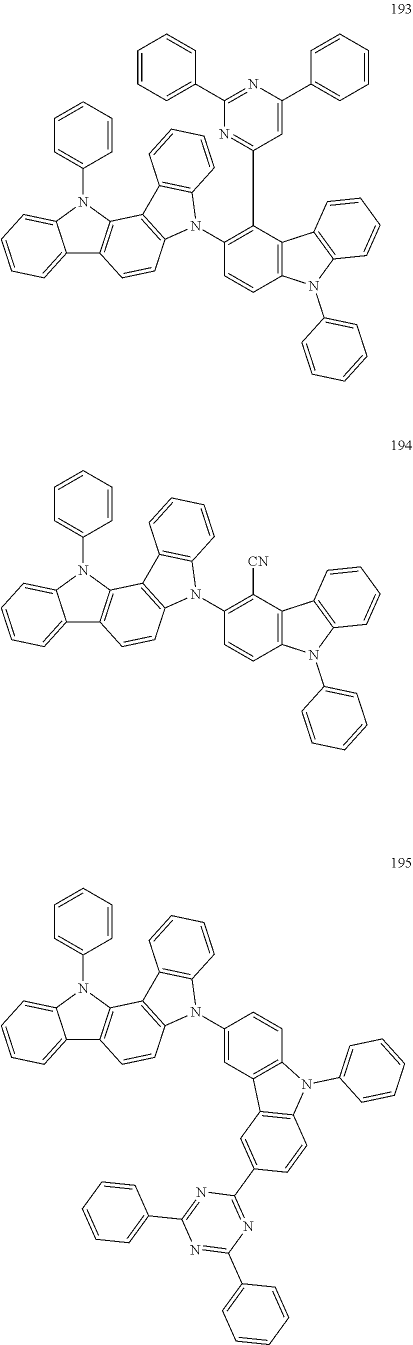 [SMILES: c1ccc(-c2cc(-c3c(-n4c5ccccc5c5c4ccc4c6ccccc6n(-c6ccccc6)c45)ccc4c3c3ccccc3n4-c3ccccc3)nc(-c3ccccc3)n2)cc1 ; N#Cc1c(-n2c3ccccc3c3c2ccc2c4ccccc4n(-c4ccccc4)c23)ccc2c1c1ccccc1n2-c1ccccc1 ; c1ccc(-c2nc(-c3ccccc3)nc(-c3ccc4c(c3)c3cc(-n5c6ccccc6c6c5ccc5c7ccccc7n(-c7ccccc7)c56)ccc3n4-c3ccccc3)n2)cc1]

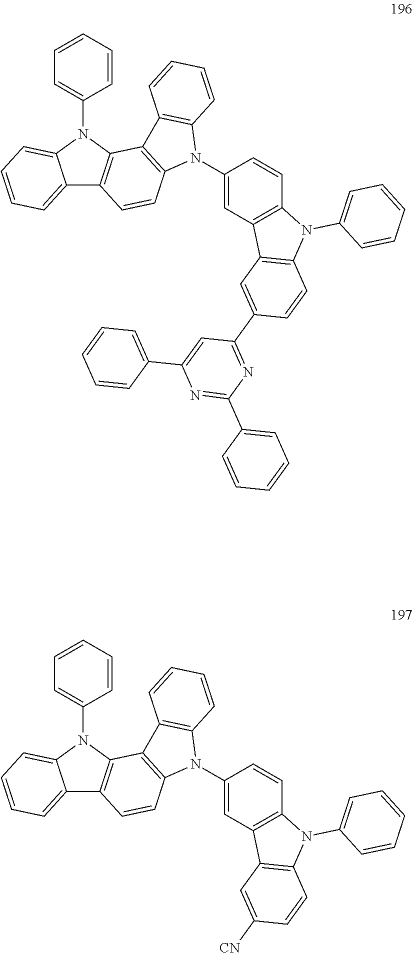

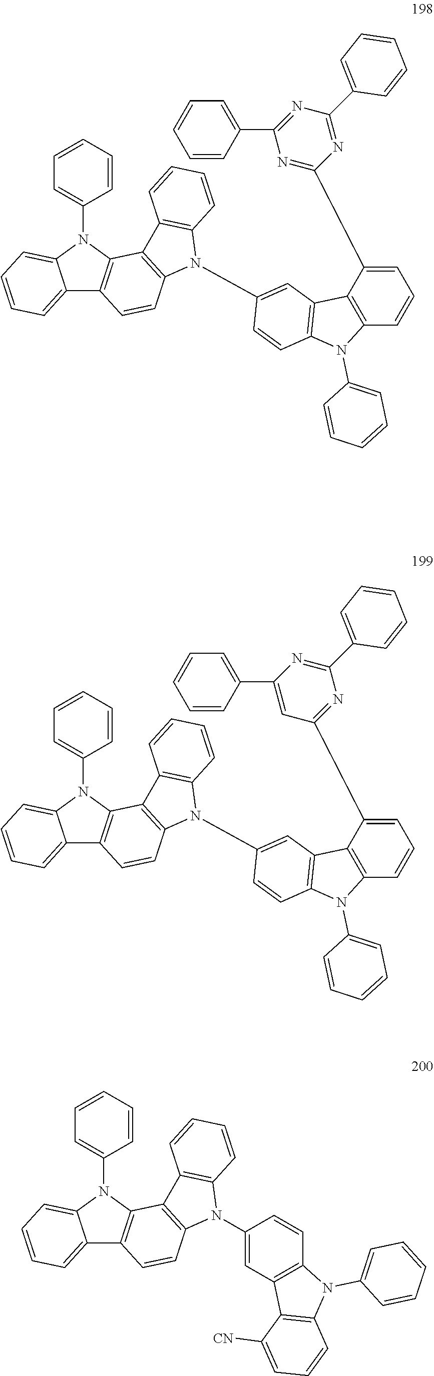

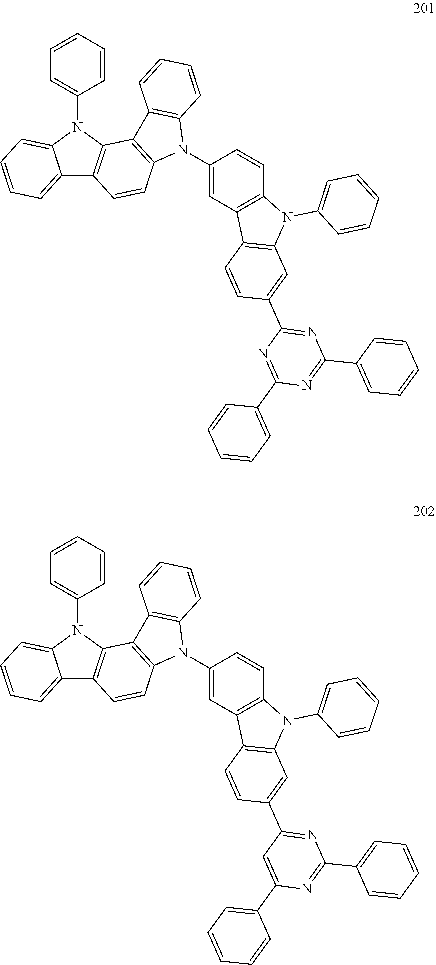 [SMILES: c1ccc(-c2nc(-c3ccccc3)nc(-c3ccc4c5cc(-n6c7ccccc7c7c6ccc6c8ccccc8n(-c8ccccc8)c67)ccc5n(-c5ccccc5)c4c3)n2)cc1 ; c1ccc(-c2cc(-c3ccc4c5cc(-n6c7ccccc7c7c6ccc6c8ccccc8n(-c8ccccc8)c67)ccc5n(-c5ccccc5)c4c3)nc(-c3ccccc3)n2)cc1]

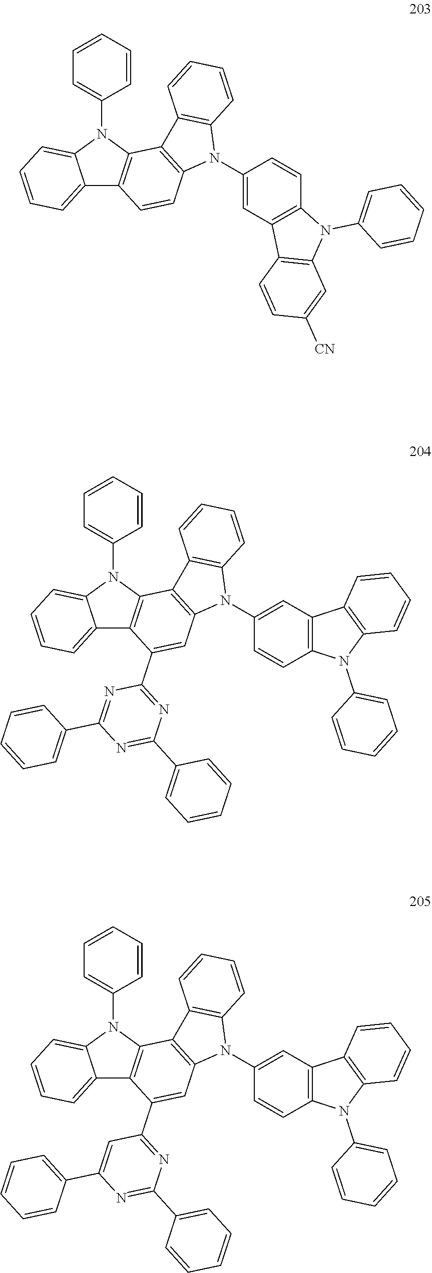 [SMILES: N#Cc1ccc2c3cc(-n4c5ccccc5c5c4ccc4c6ccccc6n(-c6ccccc6)c45)ccc3n(-c3ccccc3)c2c1 ; c1ccc(-c2nc(-c3ccccc3)nc(-c3cc4c(c5ccccc5n4-c4ccc5c(c4)c4ccccc4n5-c4ccccc4)c4c3c3ccccc3n4-c3ccccc3)n2)cc1 ; c1ccc(-c2cc(-c3cc4c(c5ccccc5n4-c4ccc5c(c4)c4ccccc4n5-c4ccccc4)c4c3c3ccccc3n4-c3ccccc3)nc(-c3ccccc3)n2)cc1]

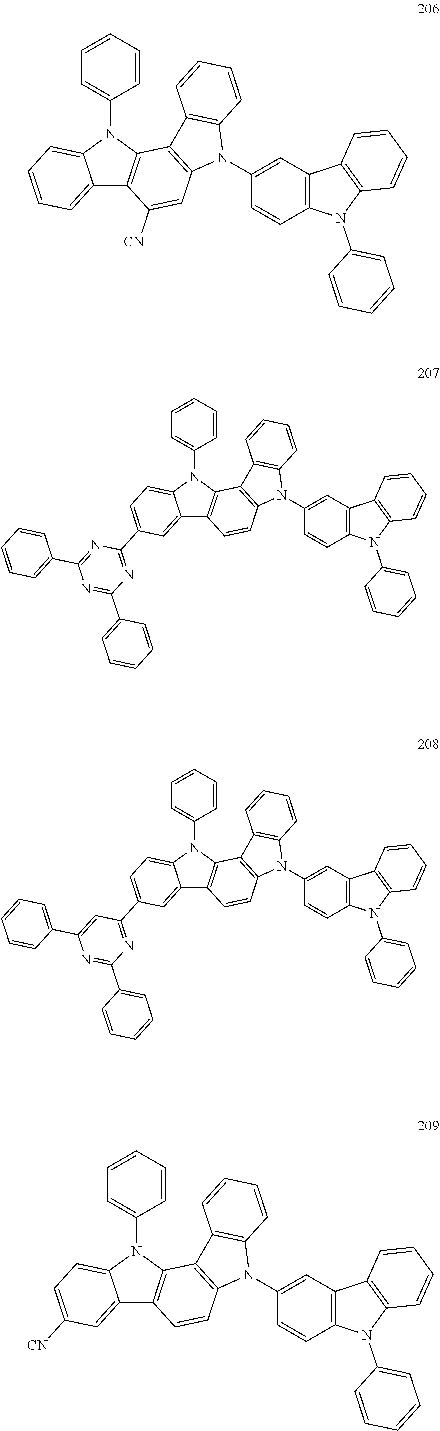

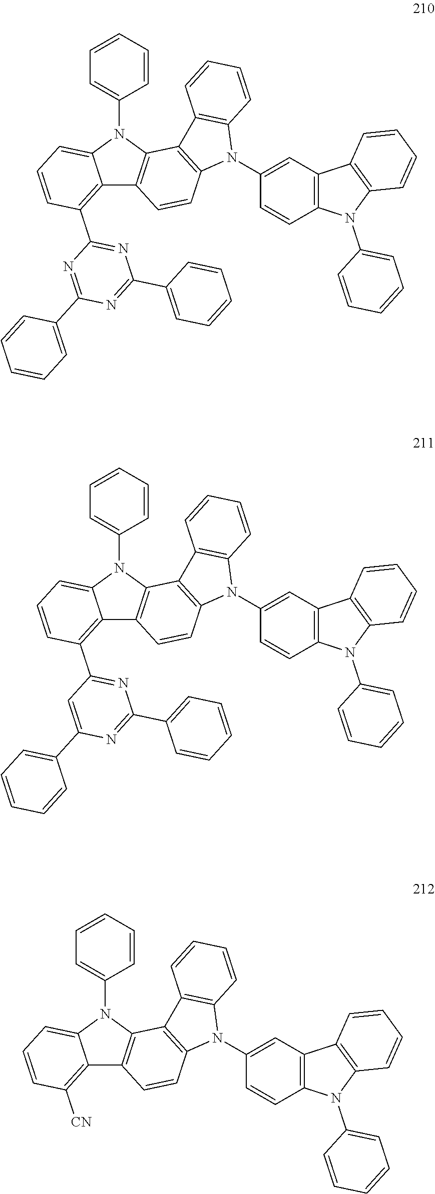 [SMILES: c1ccc(-c2nc(-c3ccccc3)nc(-c3cccc4c3c3ccc5c(c6ccccc6n5-c5ccc6c(c5)c5ccccc5n6-c5ccccc5)c3n4-c3ccccc3)n2)cc1 ; c1ccc(-c2cc(-c3cccc4c3c3ccc5c(c6ccccc6n5-c5ccc6c(c5)c5ccccc5n6-c5ccccc5)c3n4-c3ccccc3)nc(-c3ccccc3)n2)cc1 ; N#Cc1cccc2c1c1ccc3c(c4ccccc4n3-c3ccc4c(c3)c3ccccc3n4-c3ccccc3)c1n2-c1ccccc1]

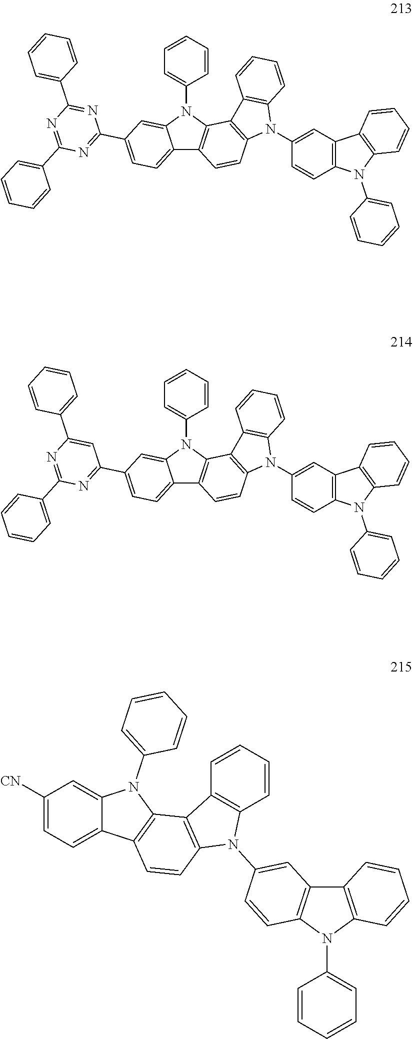

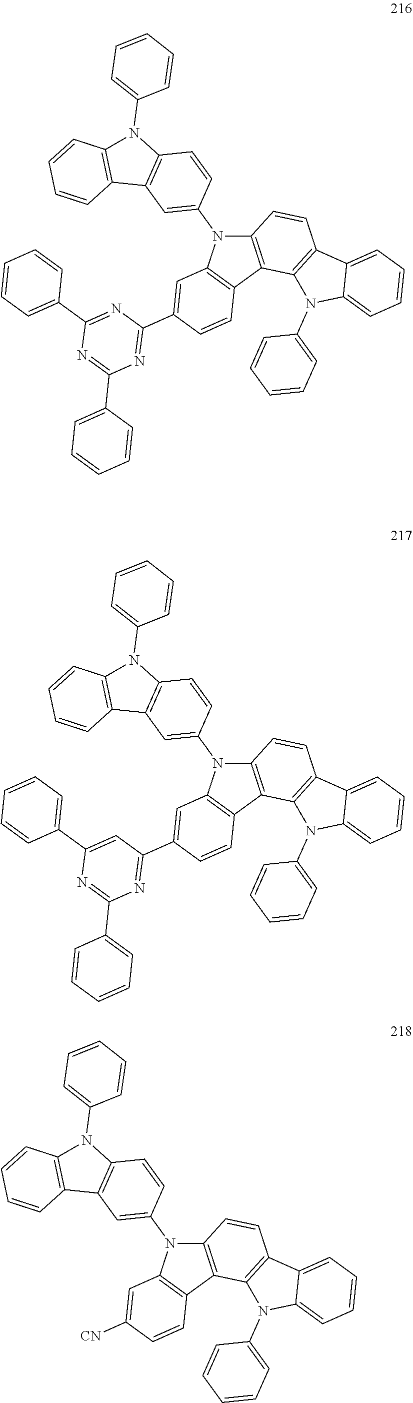

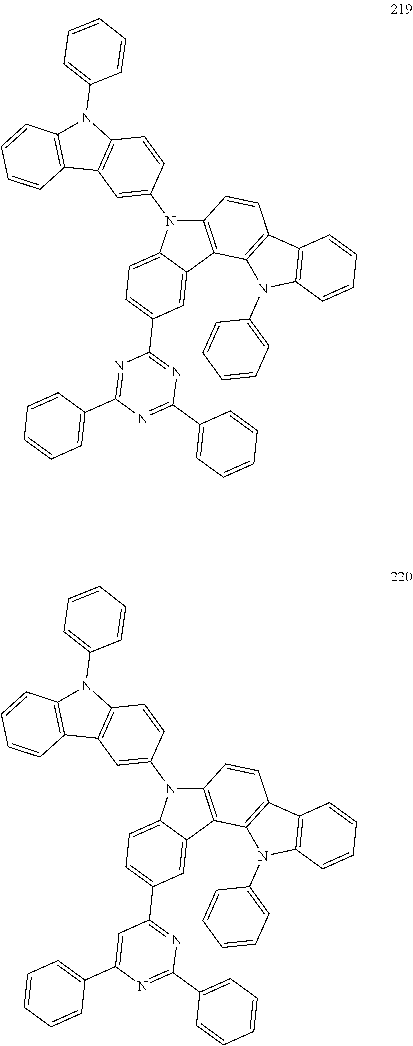 [SMILES: c1ccc(-c2nc(-c3ccccc3)nc(-c3ccc4c(c3)c3c(ccc5c6ccccc6n(-c6ccccc6)c53)n4-c3ccc4c(c3)c3ccccc3n4-c3ccccc3)n2)cc1 ; c1ccc(-c2cc(-c3ccc4c(c3)c3c(ccc5c6ccccc6n(-c6ccccc6)c53)n4-c3ccc4c(c3)c3ccccc3n4-c3ccccc3)nc(-c3ccccc3)n2)cc1]

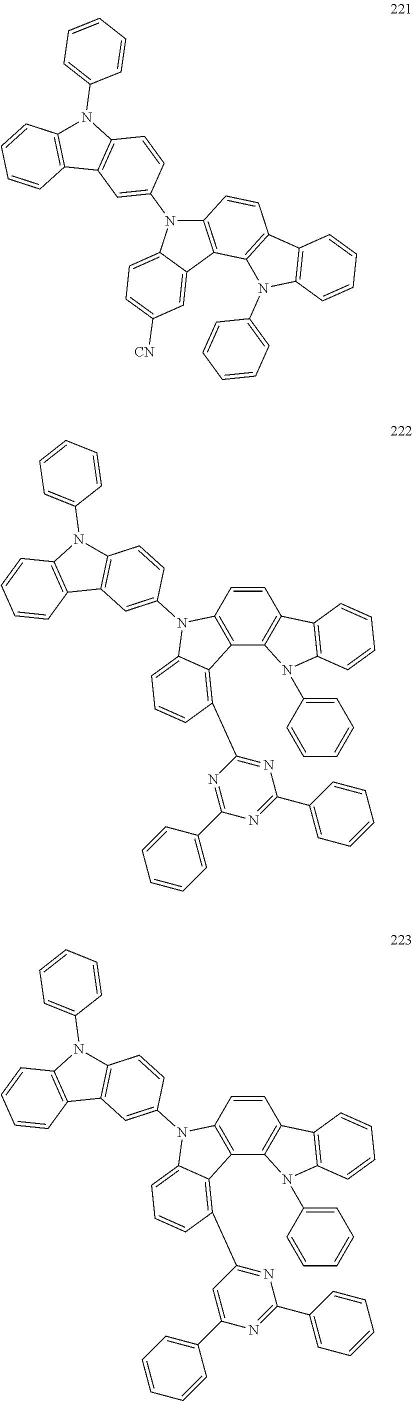

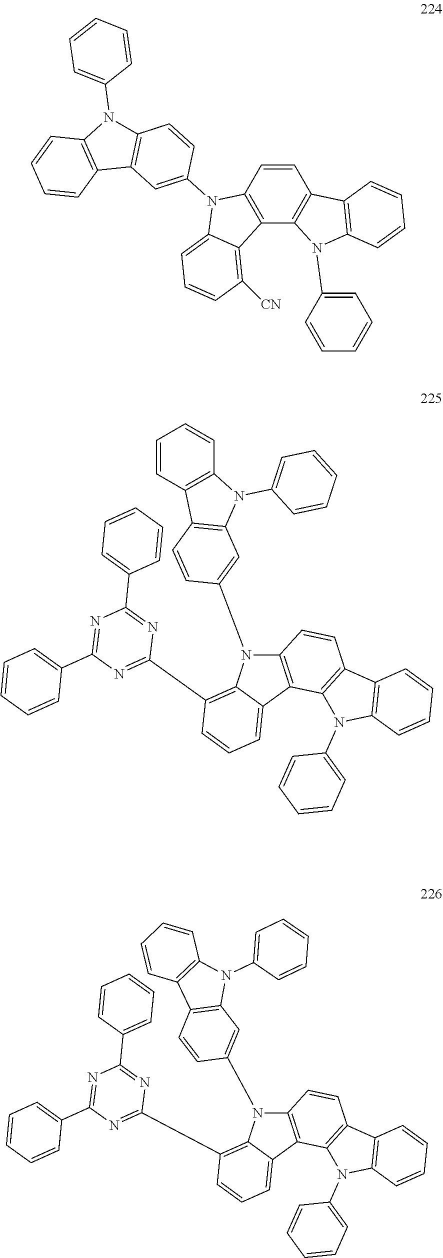 [SMILES: N#Cc1cccc2c1c1c(ccc3c4ccccc4n(-c4ccccc4)c31)n2-c1ccc2c(c1)c1ccccc1n2-c1ccccc1 ; c1ccc(-c2nc(-c3ccccc3)nc(-c3cccc4c5c(ccc6c7ccccc7n(-c7ccccc7)c65)n(-c5ccc6c7ccccc7n(-c7ccccc7)c6c5)c34)n2)cc1 ; c1ccc(-c2nc(-c3ccccc3)nc(-c3cccc4c5c(ccc6c7ccccc7n(-c7ccccc7)c65)n(-c5ccc6c7ccccc7n(-c7ccccc7)c6c5)c34)n2)cc1]

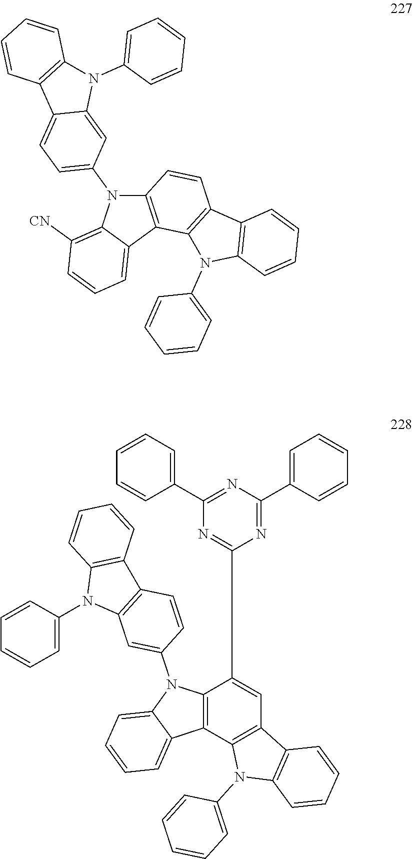

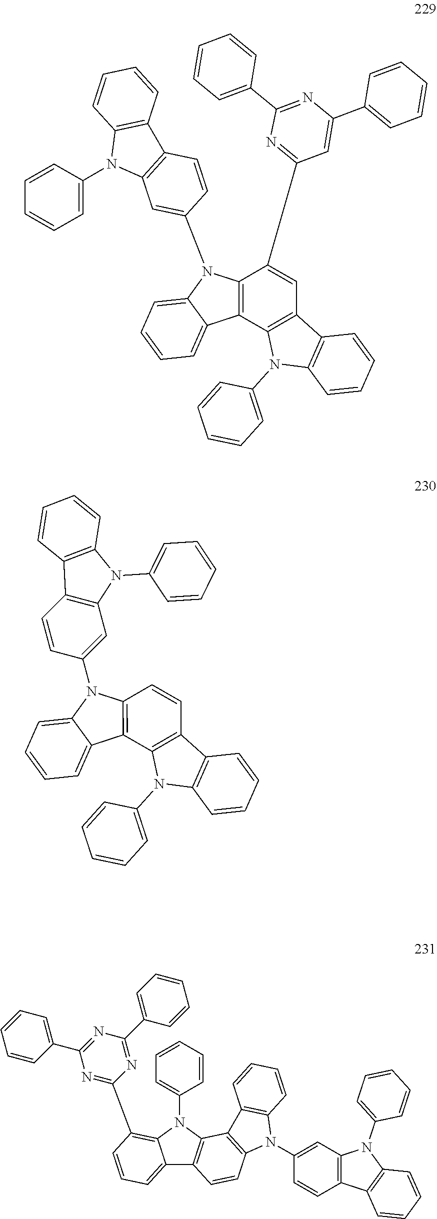 [SMILES: c1ccc(-c2cc(-c3cc4c5ccccc5n(-c5ccccc5)c4c4c5ccccc5n(-c5ccc6c7ccccc7n(-c7ccccc7)c6c5)c34)nc(-c3ccccc3)n2)cc1 ; c1ccc(-n2c3ccccc3c3ccc(-n4c5ccccc5c5c4ccc4c6ccccc6n(-c6ccccc6)c45)cc32)cc1 ; c1ccc(-c2nc(-c3ccccc3)nc(-c3cccc4c5ccc6c(c7ccccc7n6-c6ccc7c8ccccc8n(-c8ccccc8)c7c6)c5n(-c5ccccc5)c34)n2)cc1]

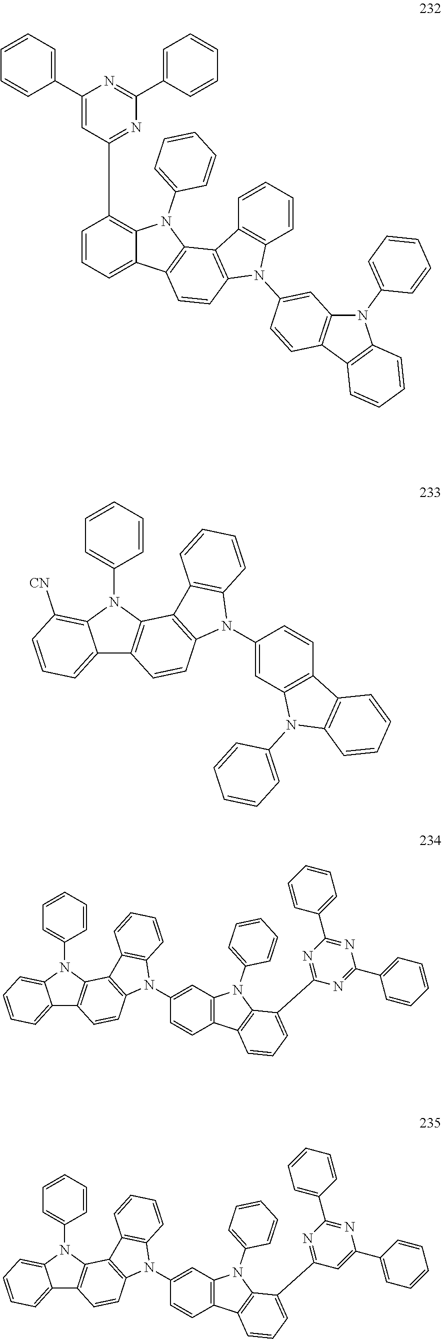

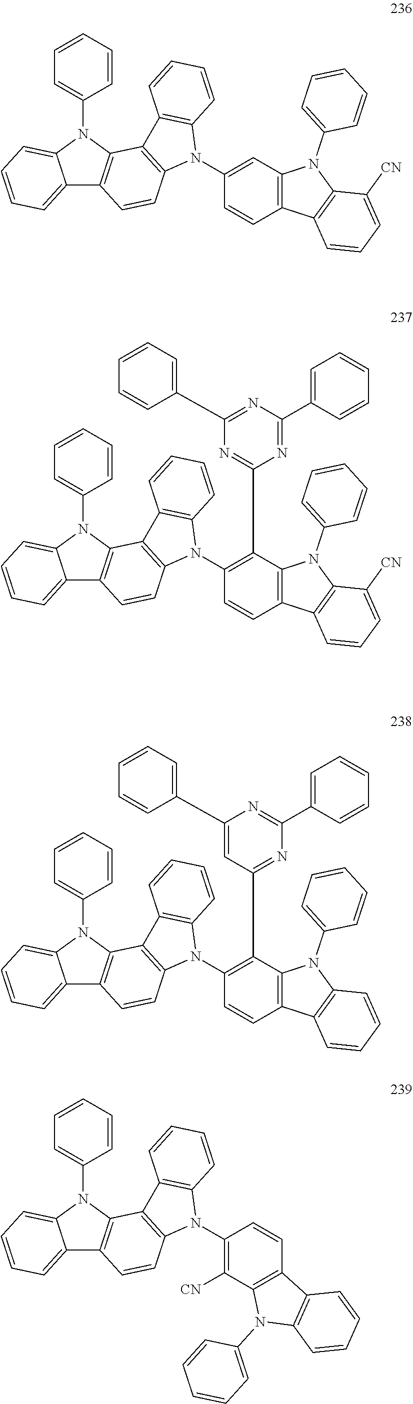

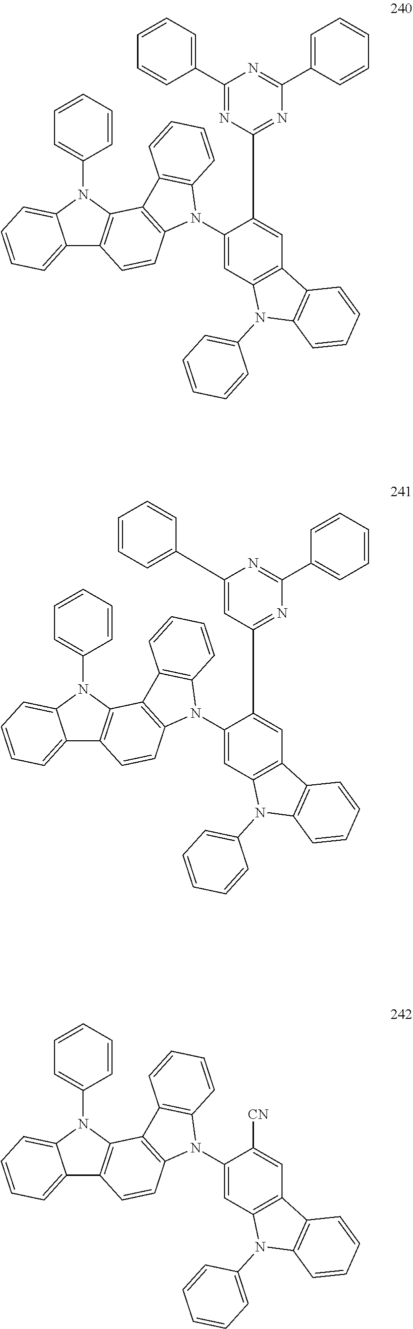 [SMILES: c1ccc(-c2nc(-c3ccccc3)nc(-c3cc4c5ccccc5n(-c5ccccc5)c4cc3-n3c4ccccc4c4c3ccc3c5ccccc5n(-c5ccccc5)c34)n2)cc1 ; c1ccc(-c2cc(-c3cc4c5ccccc5n(-c5ccccc5)c4cc3-n3c4ccccc4c4c3ccc3c5ccccc5n(-c5ccccc5)c34)nc(-c3ccccc3)n2)cc1 ; N#Cc1cc2c3ccccc3n(-c3ccccc3)c2cc1-n1c2ccccc2c2c1ccc1c3ccccc3n(-c3ccccc3)c12]

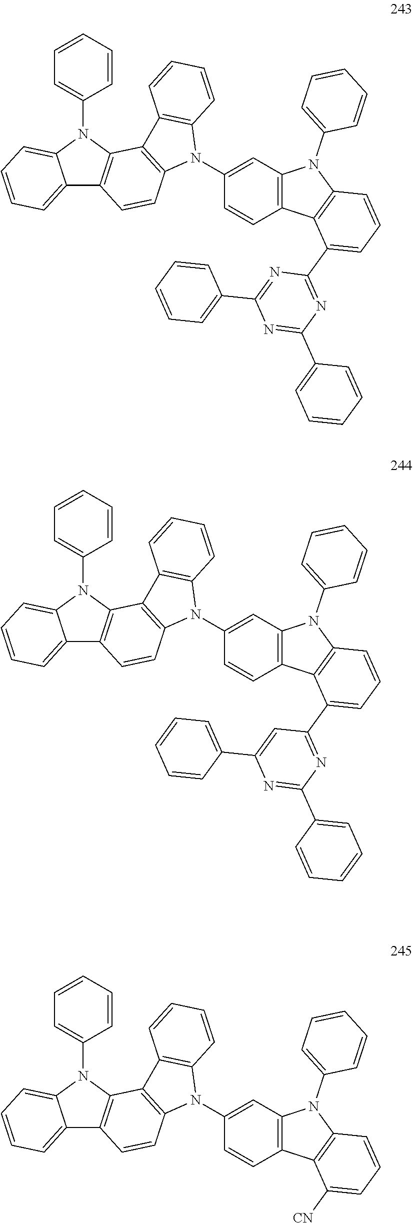

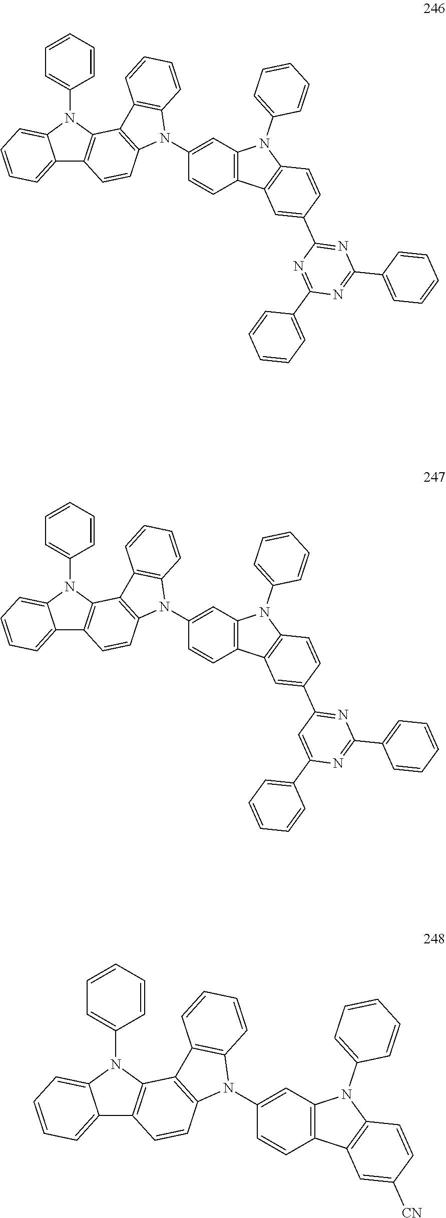 [SMILES: c1ccc(-c2nc(-c3ccccc3)nc(-c3ccc4c(c3)c3ccc(-n5c6ccccc6c6c5ccc5c7ccccc7n(-c7ccccc7)c56)cc3n4-c3ccccc3)n2)cc1 ; c1ccc(-c2cc(-c3ccc4c(c3)c3ccc(-n5c6ccccc6c6c5ccc5c7ccccc7n(-c7ccccc7)c56)cc3n4-c3ccccc3)nc(-c3ccccc3)n2)cc1 ; N#Cc1ccc2c(c1)c1ccc(-n3c4ccccc4c4c3ccc3c5ccccc5n(-c5ccccc5)c34)cc1n2-c1ccccc1]

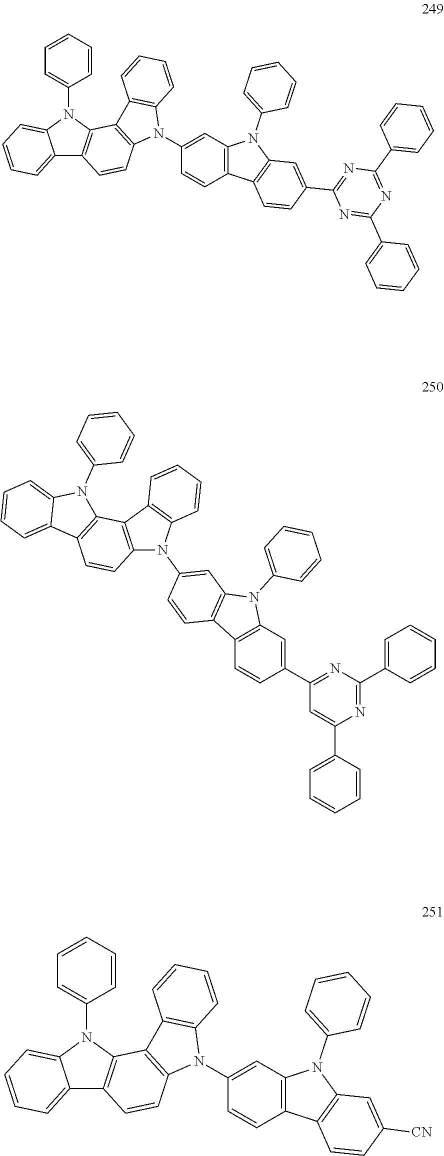 [SMILES: c1ccc(-c2nc(-c3ccccc3)nc(-c3ccc4c5ccc(-n6c7ccccc7c7c6ccc6c8ccccc8n(-c8ccccc8)c67)cc5n(-c5ccccc5)c4c3)n2)cc1 ; c1ccc(-c2cc(-c3ccc4c5ccc(-n6c7ccccc7c7c6ccc6c8ccccc8n(-c8ccccc8)c67)cc5n(-c5ccccc5)c4c3)nc(-c3ccccc3)n2)cc1 ; N#Cc1ccc2c3ccc(-n4c5ccccc5c5c4ccc4c6ccccc6n(-c6ccccc6)c45)cc3n(-c3ccccc3)c2c1]

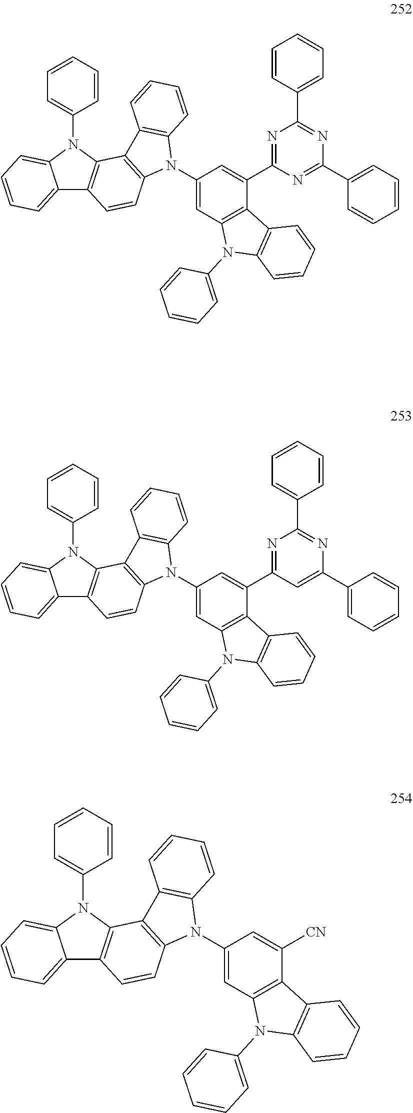 [SMILES: c1ccc(-c2nc(-c3ccccc3)nc(-c3cc(-n4c5ccccc5c5c4ccc4c6ccccc6n(-c6ccccc6)c45)cc4c3c3ccccc3n4-c3ccccc3)n2)cc1 ; c1ccc(-c2cc(-c3cc(-n4c5ccccc5c5c4ccc4c6ccccc6n(-c6ccccc6)c45)cc4c3c3ccccc3n4-c3ccccc3)nc(-c3ccccc3)n2)cc1 ; N#Cc1cc(-n2c3ccccc3c3c2ccc2c4ccccc4n(-c4ccccc4)c23)cc2c1c1ccccc1n2-c1ccccc1]

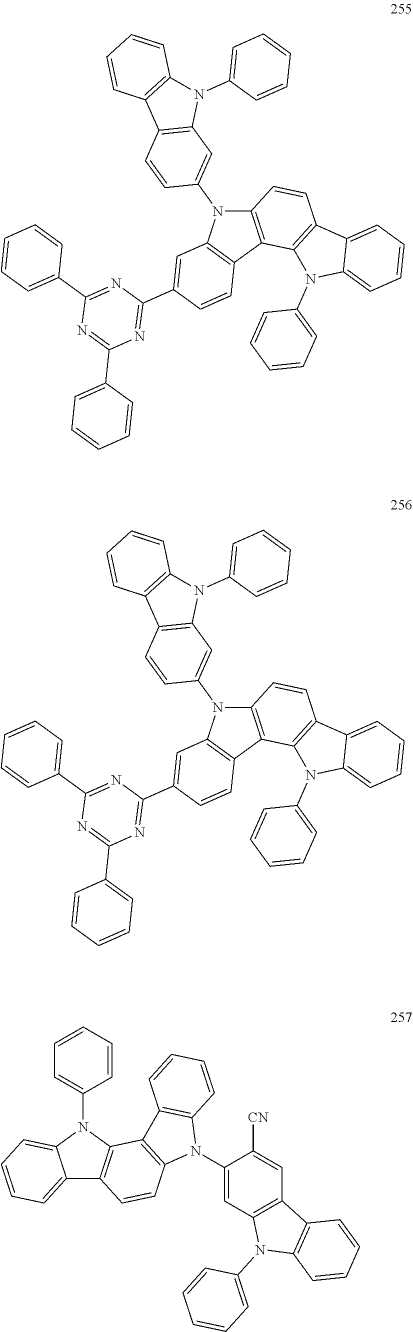 [SMILES: c1ccc(-c2nc(-c3ccccc3)nc(-c3ccc4c5c(ccc6c7ccccc7n(-c7ccccc7)c65)n(-c5ccc6c7ccccc7n(-c7ccccc7)c6c5)c4c3)n2)cc1 ; c1ccc(-c2nc(-c3ccccc3)nc(-c3ccc4c5c(ccc6c7ccccc7n(-c7ccccc7)c65)n(-c5ccc6c7ccccc7n(-c7ccccc7)c6c5)c4c3)n2)cc1 ; N#Cc1cc2c3ccccc3n(-c3ccccc3)c2cc1-n1c2ccccc2c2c1ccc1c3ccccc3n(-c3ccccc3)c12]

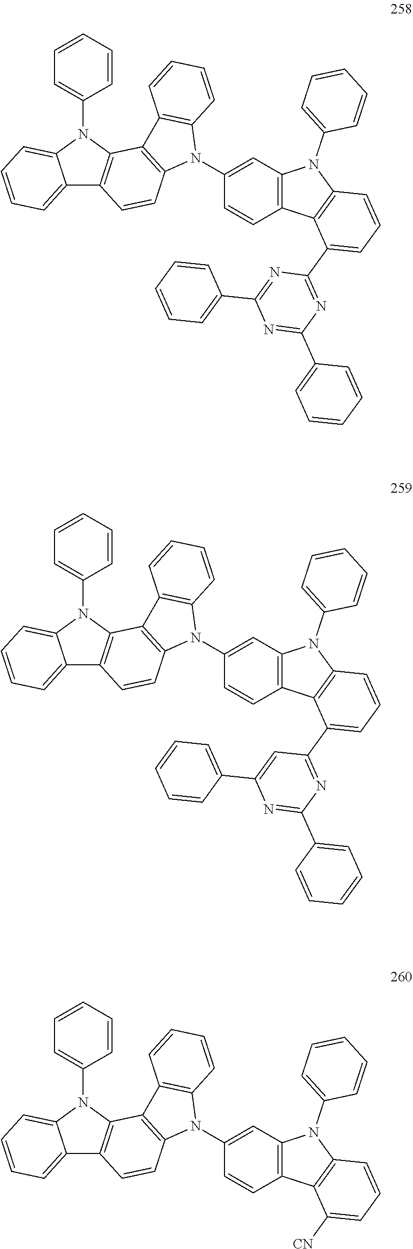

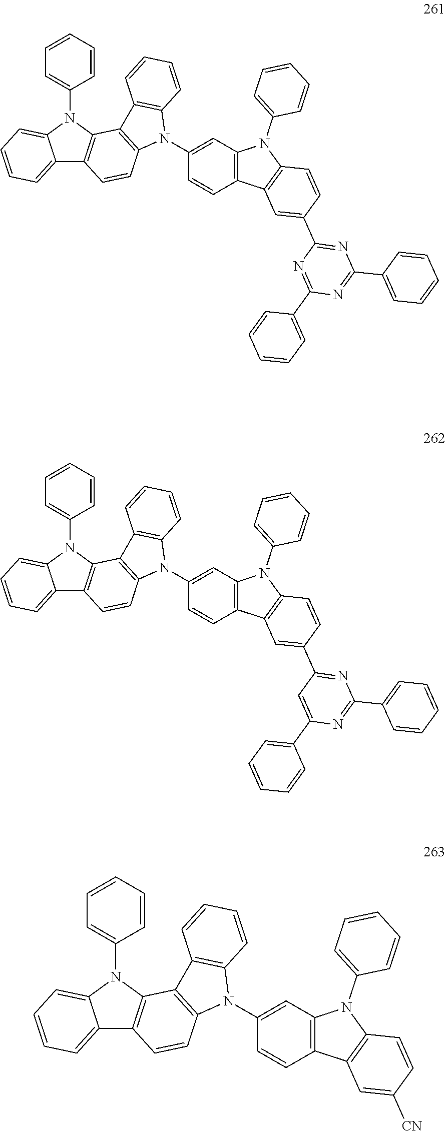 [SMILES: c1ccc(-c2nc(-c3ccccc3)nc(-c3ccc4c(c3)c3ccc(-n5c6ccccc6c6c5ccc5c7ccccc7n(-c7ccccc7)c56)cc3n4-c3ccccc3)n2)cc1 ; c1ccc(-c2cc(-c3ccc4c(c3)c3ccc(-n5c6ccccc6c6c5ccc5c7ccccc7n(-c7ccccc7)c56)cc3n4-c3ccccc3)nc(-c3ccccc3)n2)cc1 ; N#Cc1ccc2c(c1)c1ccc(-n3c4ccccc4c4c3ccc3c5ccccc5n(-c5ccccc5)c34)cc1n2-c1ccccc1]

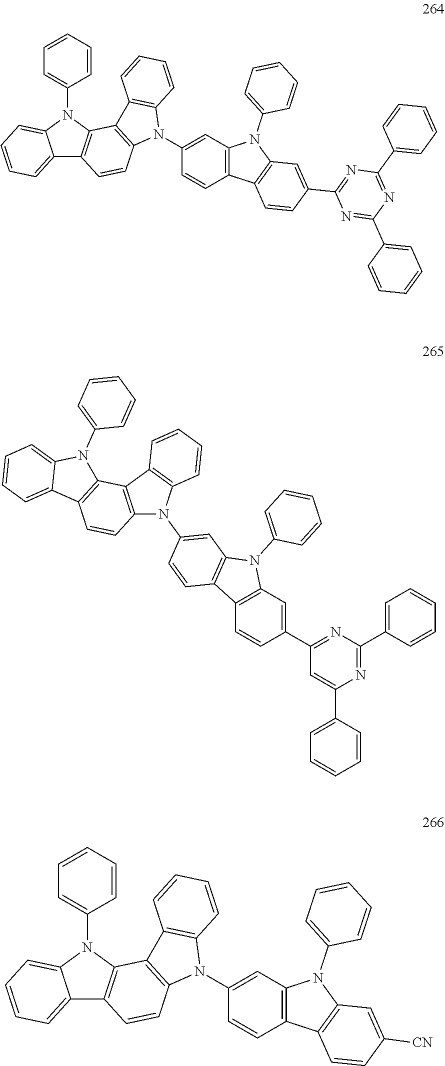 [SMILES: c1ccc(-c2nc(-c3ccccc3)nc(-c3ccc4c5ccc(-n6c7ccccc7c7c6ccc6c8ccccc8n(-c8ccccc8)c67)cc5n(-c5ccccc5)c4c3)n2)cc1 ; c1ccc(-c2cc(-c3ccc4c5ccc(-n6c7ccccc7c7c6ccc6c8ccccc8n(-c8ccccc8)c67)cc5n(-c5ccccc5)c4c3)nc(-c3ccccc3)n2)cc1 ; N#Cc1ccc2c3ccc(-n4c5ccccc5c5c4ccc4c6ccccc6n(-c6ccccc6)c45)cc3n(-c3ccccc3)c2c1]

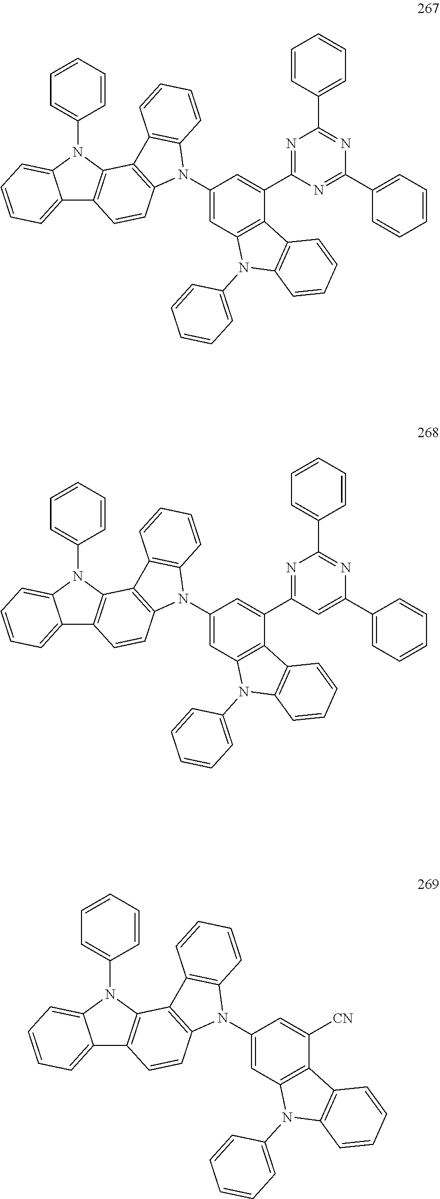 [SMILES: c1ccc(-c2nc(-c3ccccc3)nc(-c3cc(-n4c5ccccc5c5c4ccc4c6ccccc6n(-c6ccccc6)c45)cc4c3c3ccccc3n4-c3ccccc3)n2)cc1 ; c1ccc(-c2cc(-c3cc(-n4c5ccccc5c5c4ccc4c6ccccc6n(-c6ccccc6)c45)cc4c3c3ccccc3n4-c3ccccc3)nc(-c3ccccc3)n2)cc1 ; N#Cc1cc(-n2c3ccccc3c3c2ccc2c4ccccc4n(-c4ccccc4)c23)cc2c1c1ccccc1n2-c1ccccc1]

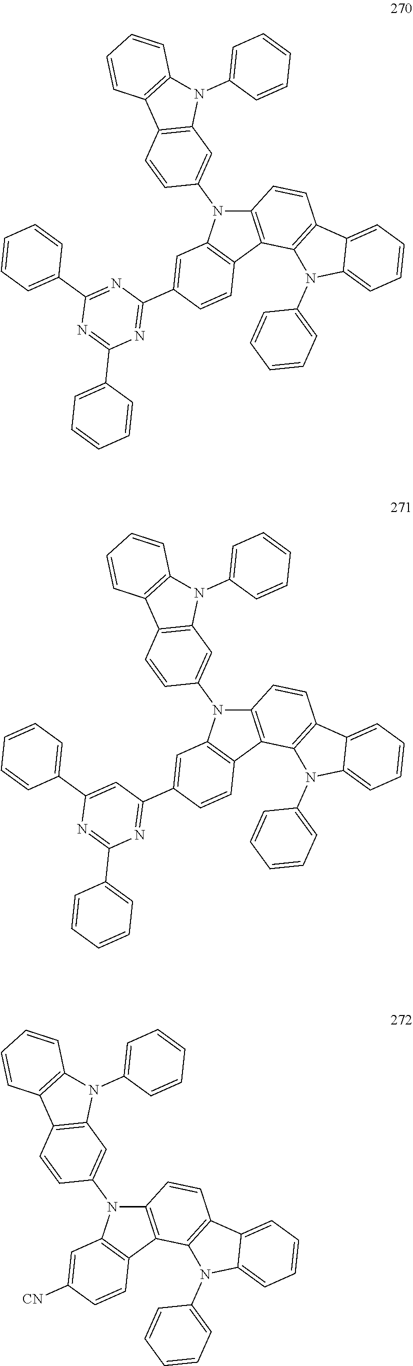

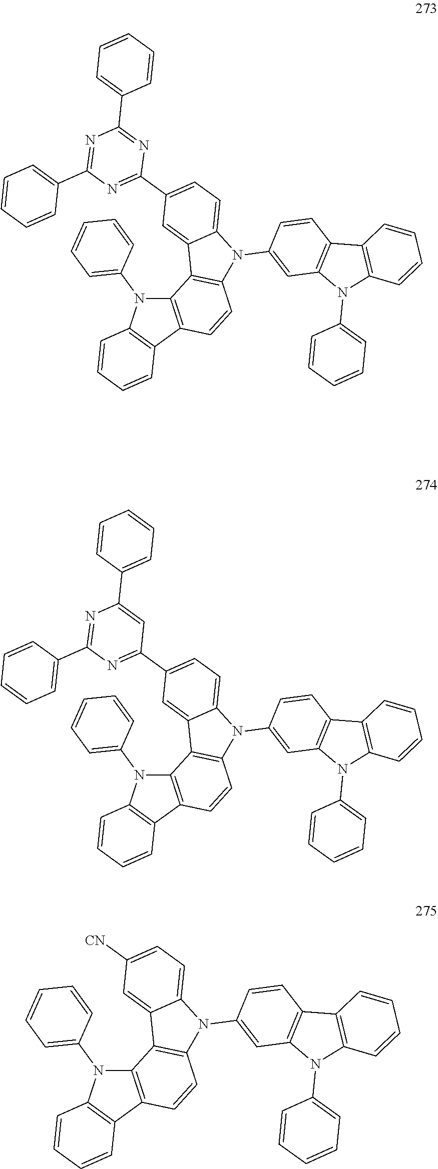

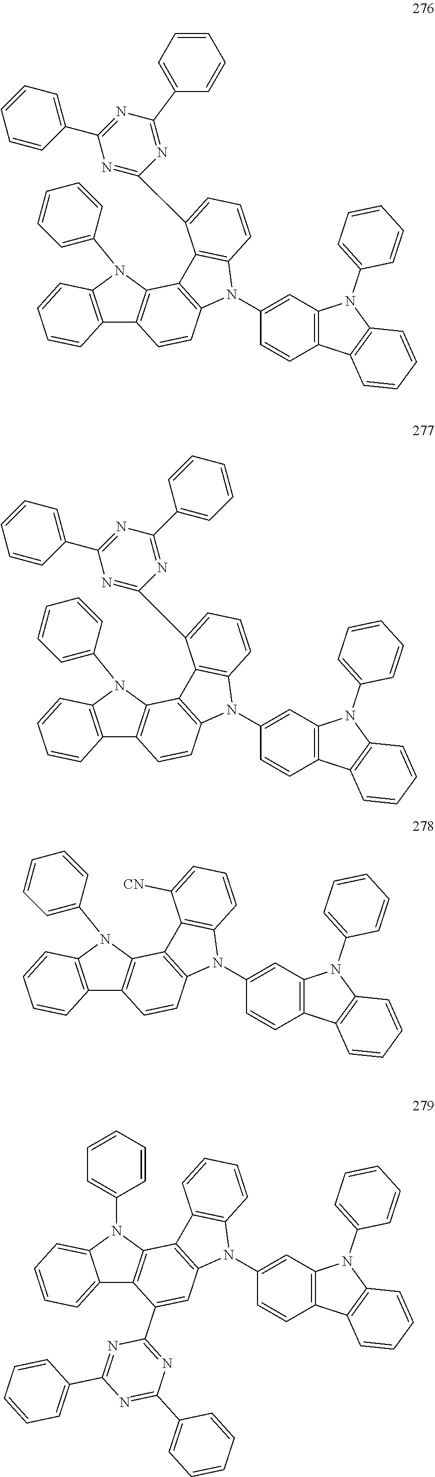

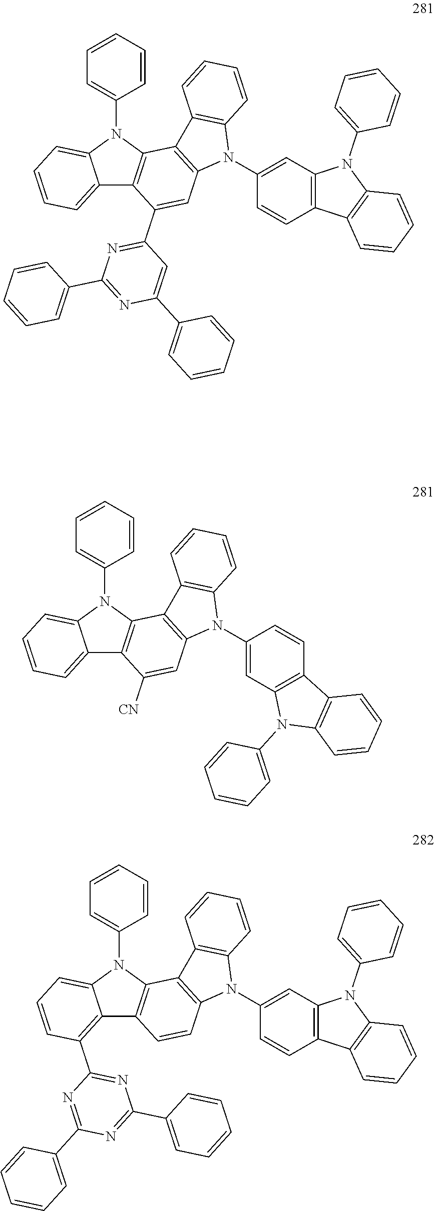

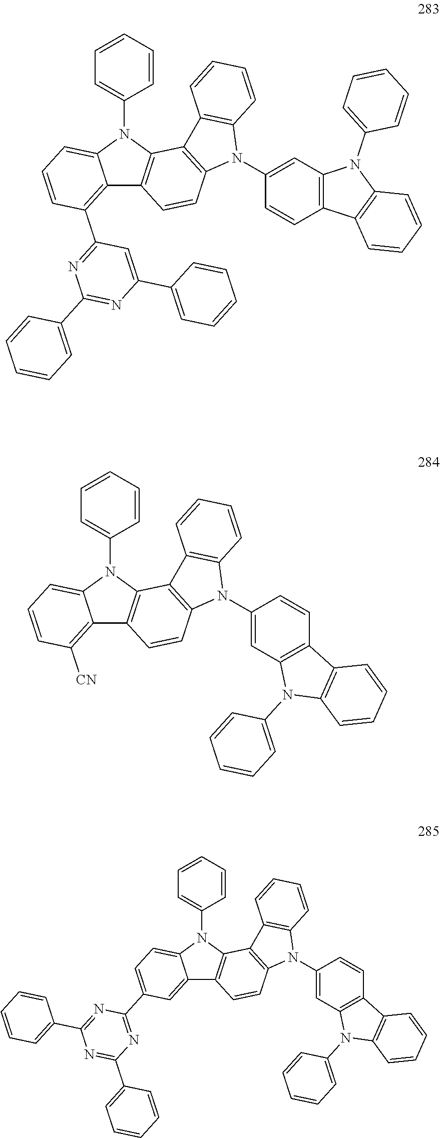 [SMILES: c1ccc(-c2cc(-c3cccc4c3c3ccc5c(c6ccccc6n5-c5ccc6c7ccccc7n(-c7ccccc7)c6c5)c3n4-c3ccccc3)nc(-c3ccccc3)n2)cc1 ; N#Cc1cccc2c1c1ccc3c(c4ccccc4n3-c3ccc4c5ccccc5n(-c5ccccc5)c4c3)c1n2-c1ccccc1 ; c1ccc(-c2nc(-c3ccccc3)nc(-c3ccc4c(c3)c3ccc5c(c6ccccc6n5-c5ccc6c7ccccc7n(-c7ccccc7)c6c5)c3n4-c3ccccc3)n2)cc1]

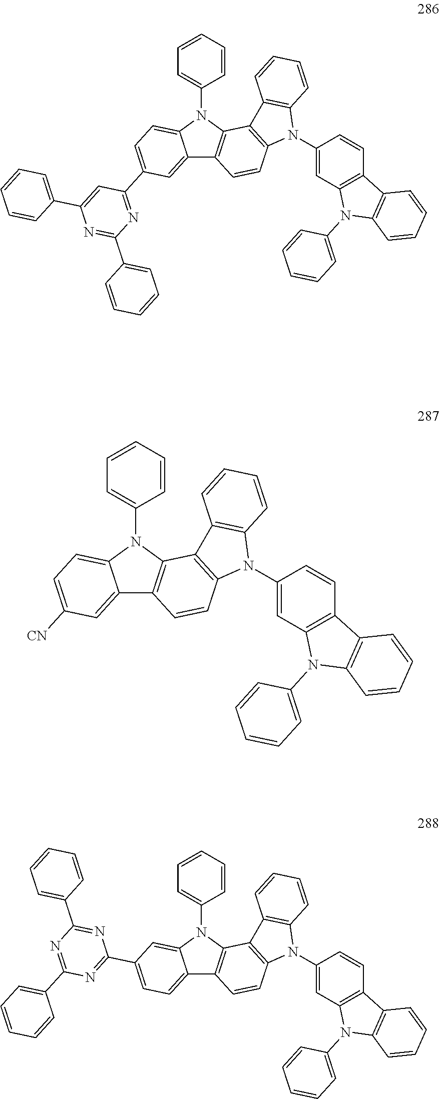

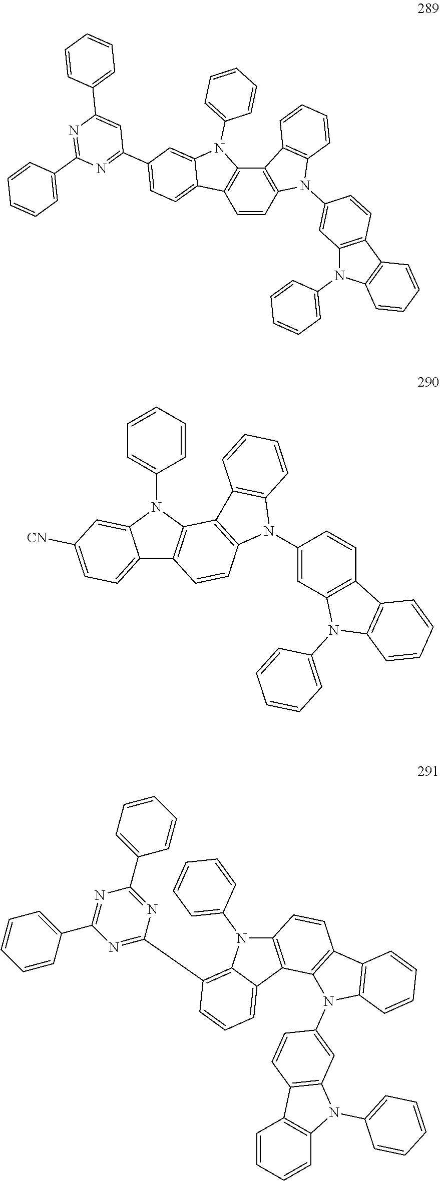

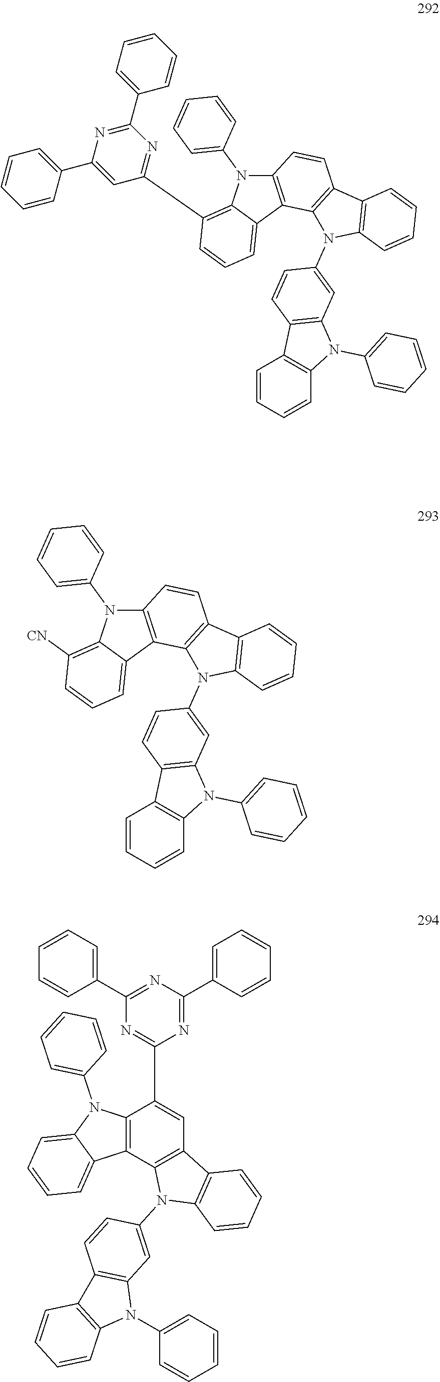

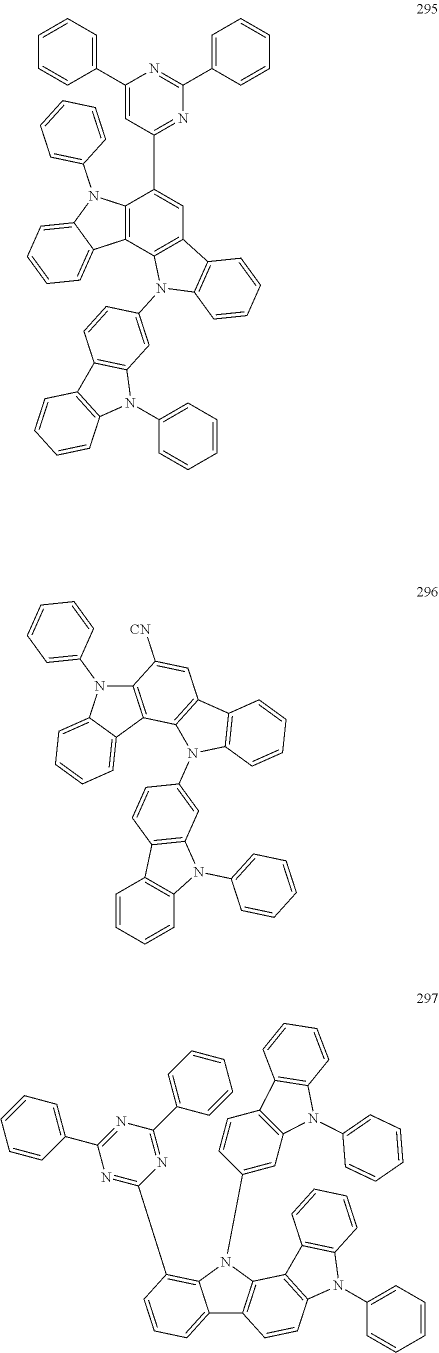

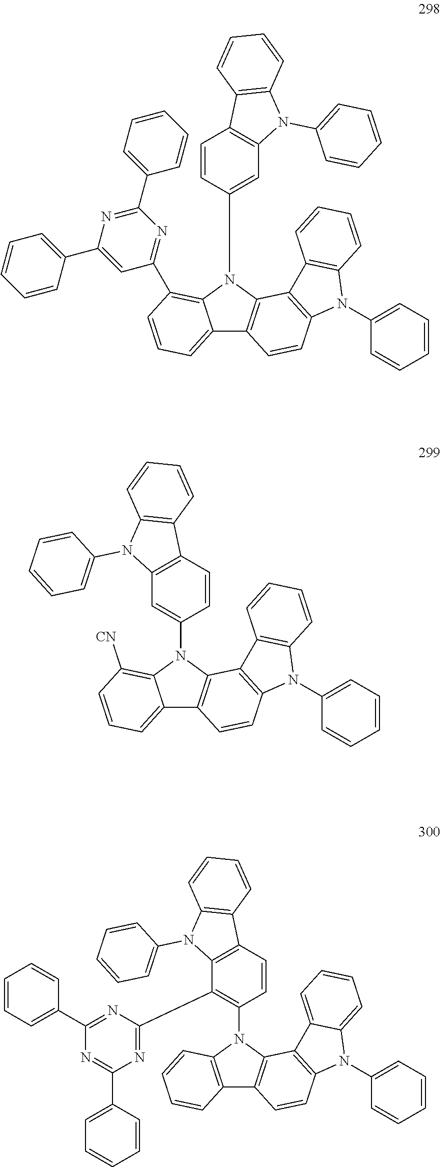

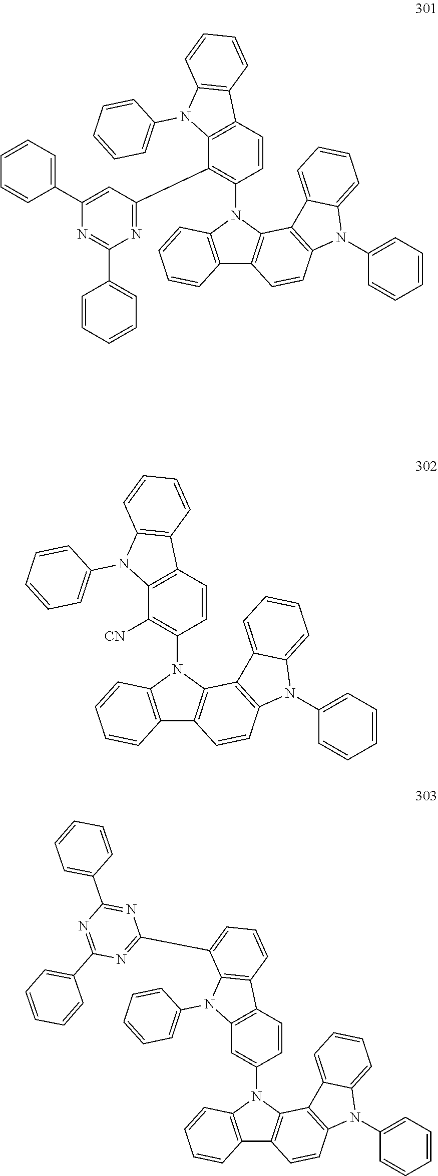

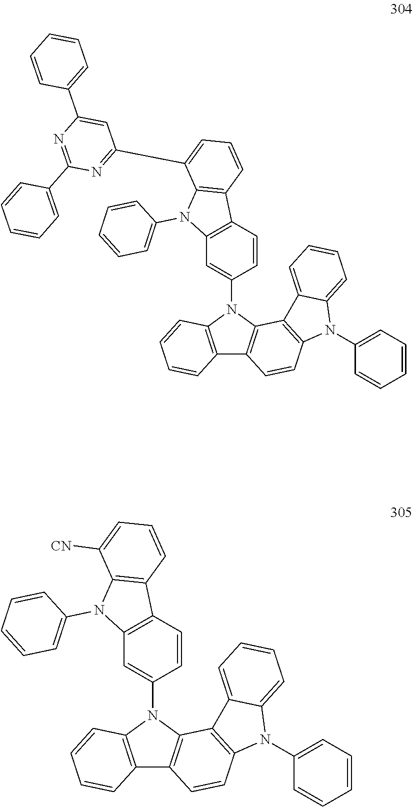

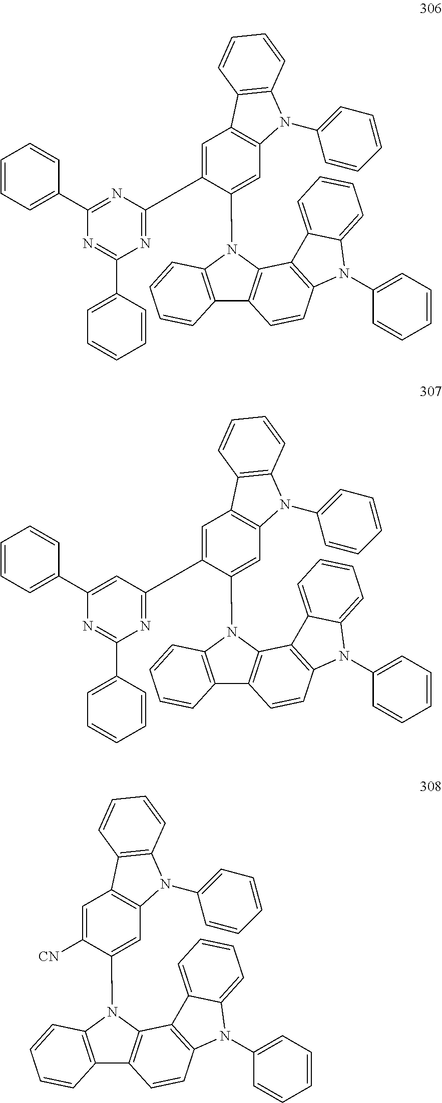

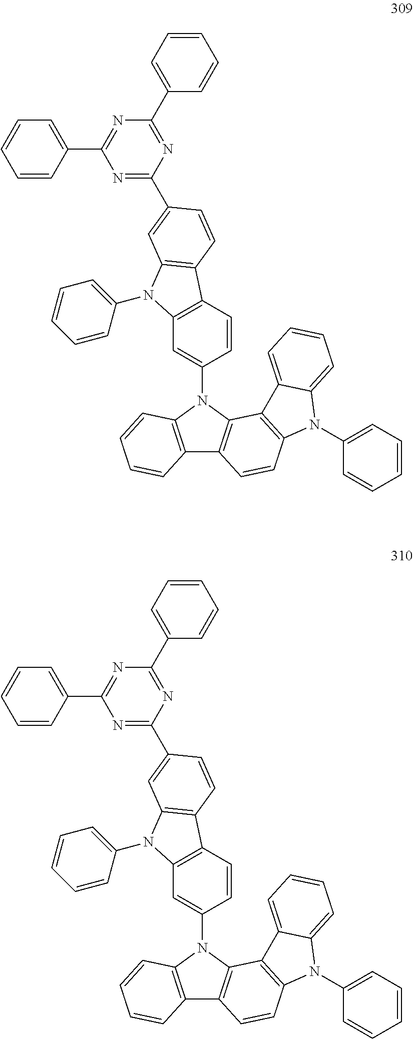 [SMILES: c1ccc(-c2nc(-c3ccccc3)nc(-c3ccc4c5ccc(-n6c7ccccc7c7ccc8c(c9ccccc9n8-c8ccccc8)c76)cc5n(-c5ccccc5)c4c3)n2)cc1 ; c1ccc(-c2nc(-c3ccccc3)nc(-c3ccc4c5ccc(-n6c7ccccc7c7ccc8c(c9ccccc9n8-c8ccccc8)c76)cc5n(-c5ccccc5)c4c3)n2)cc1]

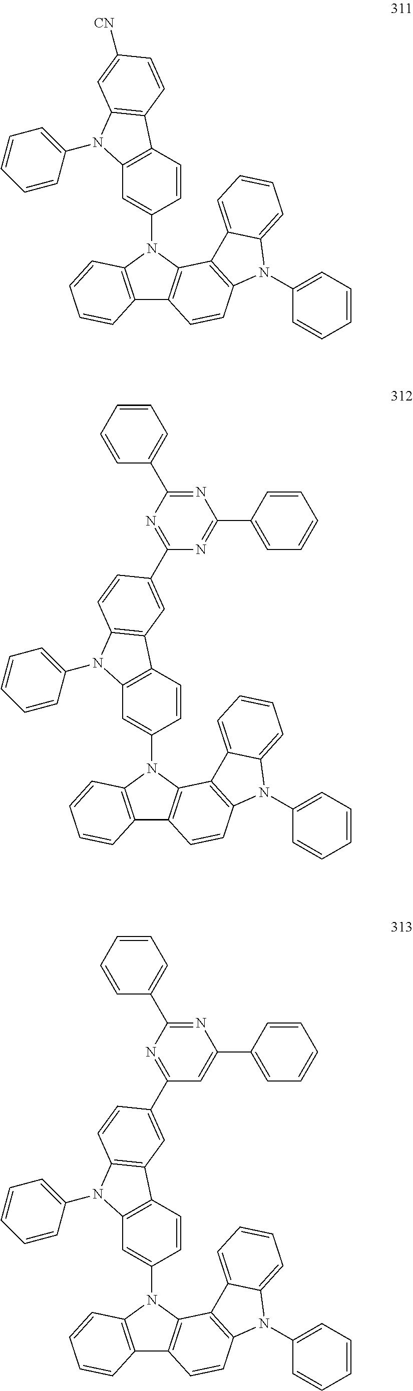

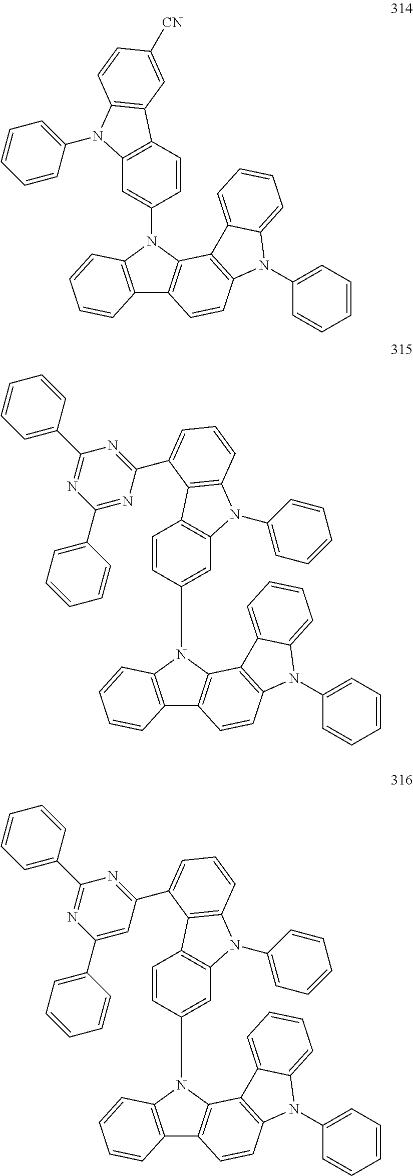 [SMILES: N#Cc1ccc2c(c1)c1ccc(-n3c4ccccc4c4ccc5c(c6ccccc6n5-c5ccccc5)c43)cc1n2-c1ccccc1 ; c1ccc(-c2nc(-c3ccccc3)nc(-c3cccc4c3c3ccc(-n5c6ccccc6c6ccc7c(c8ccccc8n7-c7ccccc7)c65)cc3n4-c3ccccc3)n2)cc1 ; c1ccc(-c2cc(-c3cccc4c3c3ccc(-n5c6ccccc6c6ccc7c(c8ccccc8n7-c7ccccc7)c65)cc3n4-c3ccccc3)nc(-c3ccccc3)n2)cc1]

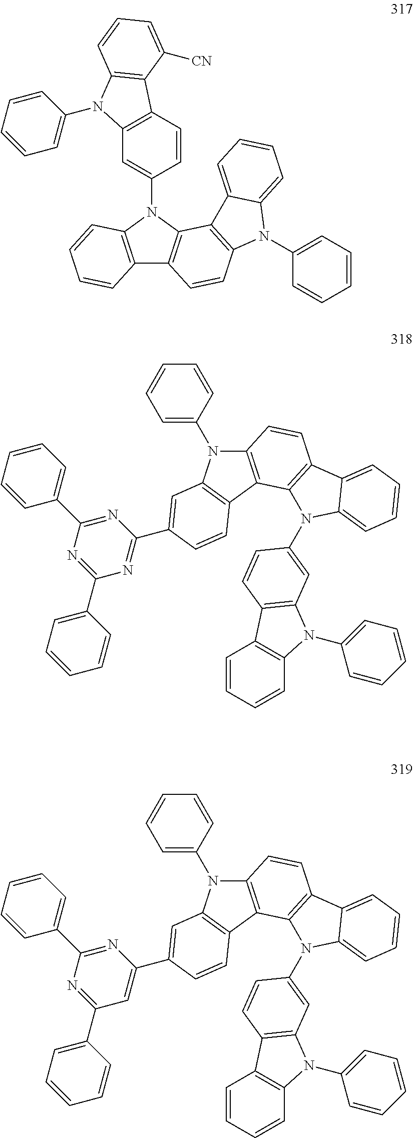 [SMILES: N#Cc1cccc2c1c1ccc(-n3c4ccccc4c4ccc5c(c6ccccc6n5-c5ccccc5)c43)cc1n2-c1ccccc1 ; c1ccc(-c2nc(-c3ccccc3)nc(-c3ccc4c5c(ccc6c7ccccc7n(-c7ccc8c9ccccc9n(-c9ccccc9)c8c7)c65)n(-c5ccccc5)c4c3)n2)cc1 ; c1ccc(-c2cc(-c3ccc4c5c(ccc6c7ccccc7n(-c7ccc8c9ccccc9n(-c9ccccc9)c8c7)c65)n(-c5ccccc5)c4c3)nc(-c3ccccc3)n2)cc1]

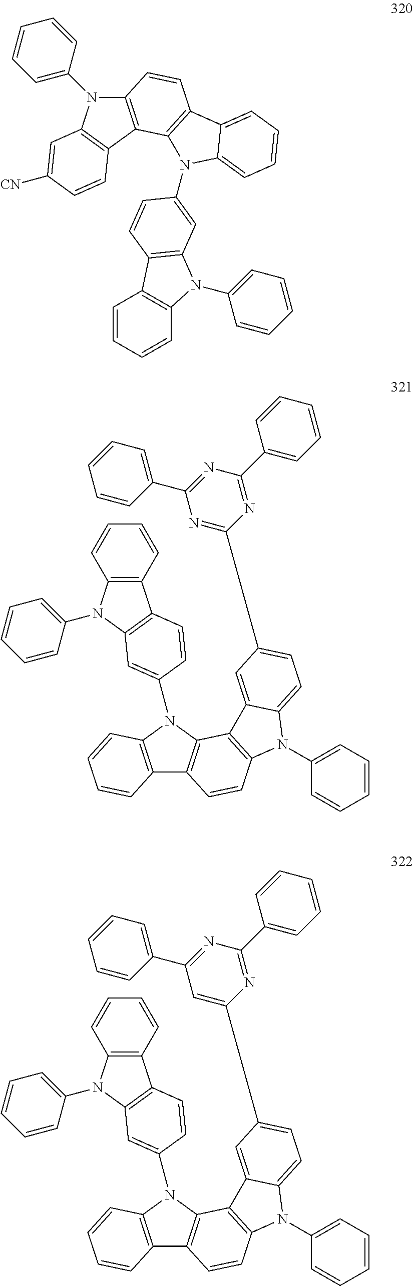

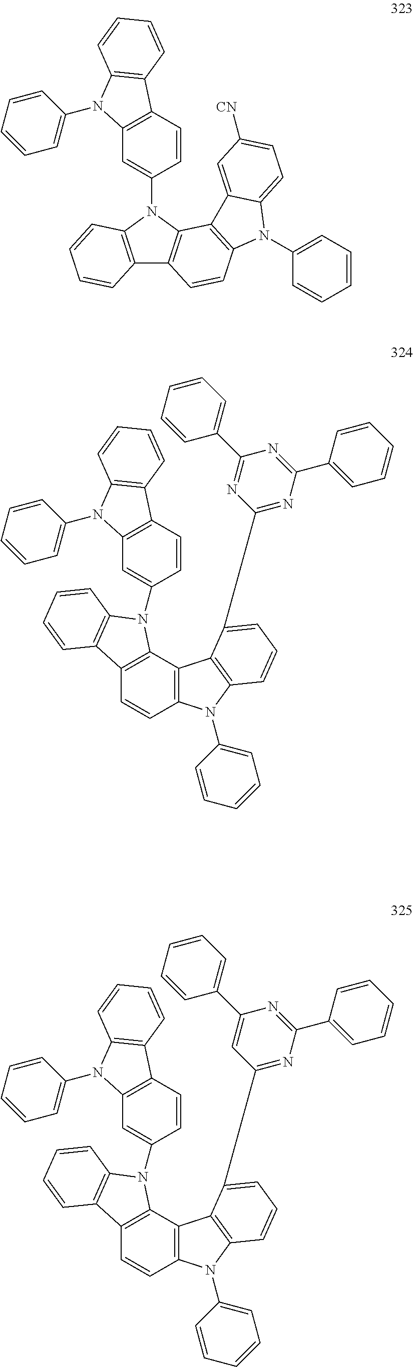

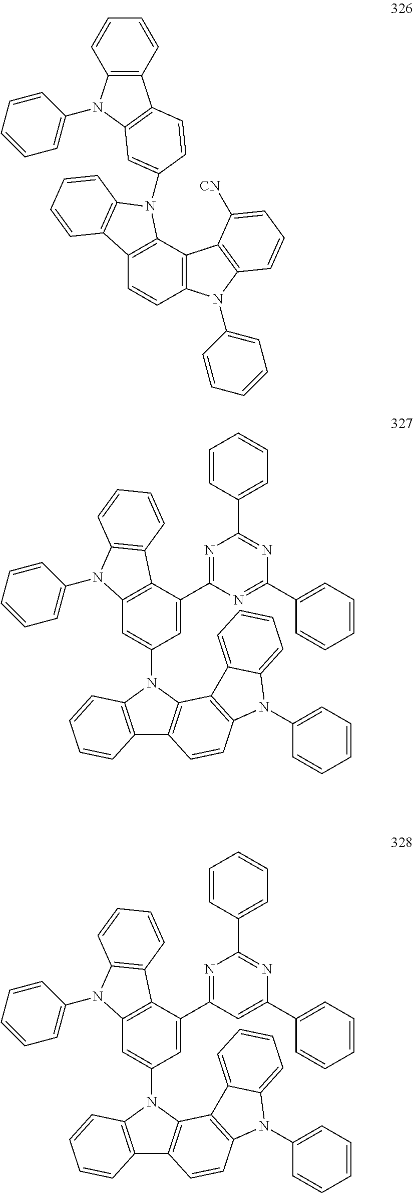

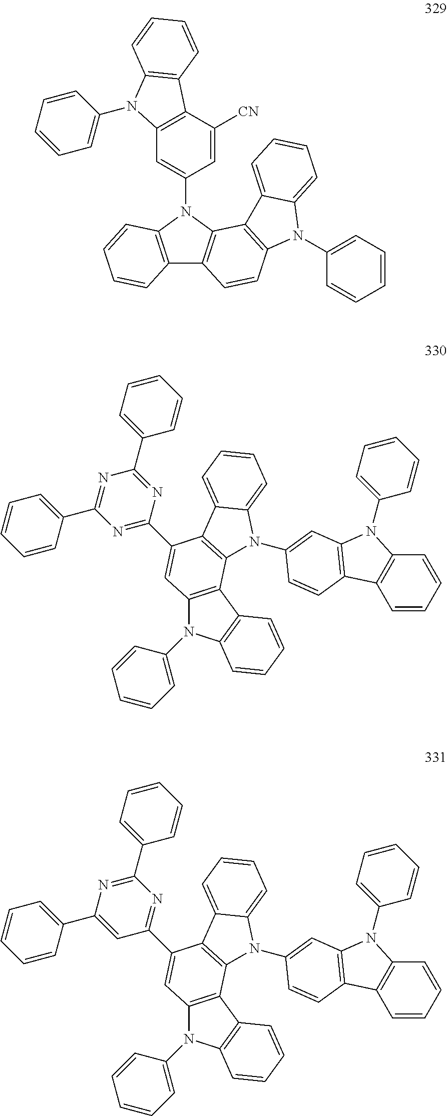 [SMILES: N#Cc1cc(-n2c3ccccc3c3ccc4c(c5ccccc5n4-c4ccccc4)c32)cc2c1c1ccccc1n2-c1ccccc1 ; c1ccc(-c2nc(-c3ccccc3)nc(-c3cc4c(c5ccccc5n4-c4ccccc4)c4c3c3ccccc3n4-c3ccc4c5ccccc5n(-c5ccccc5)c4c3)n2)cc1 ; c1ccc(-c2cc(-c3cc4c(c5ccccc5n4-c4ccccc4)c4c3c3ccccc3n4-c3ccc4c5ccccc5n(-c5ccccc5)c4c3)nc(-c3ccccc3)n2)cc1]

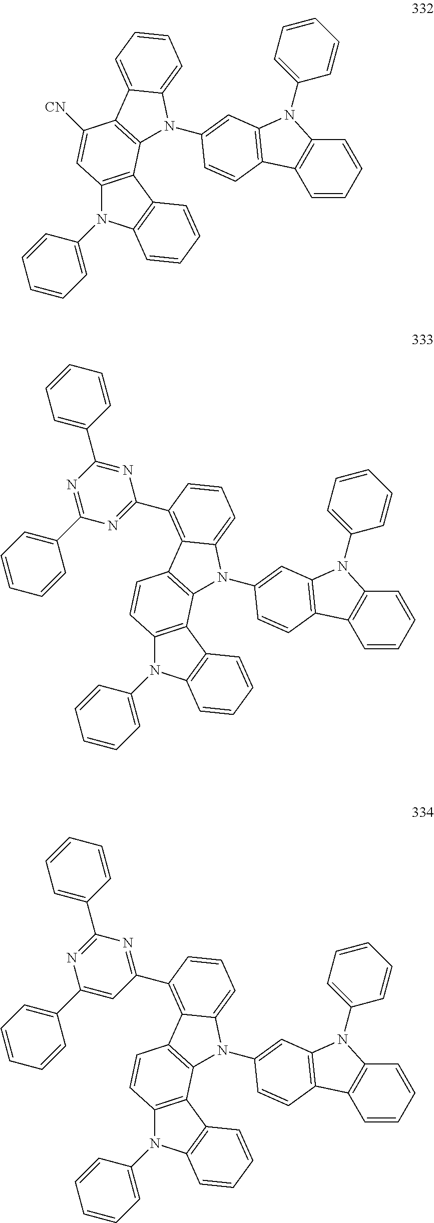

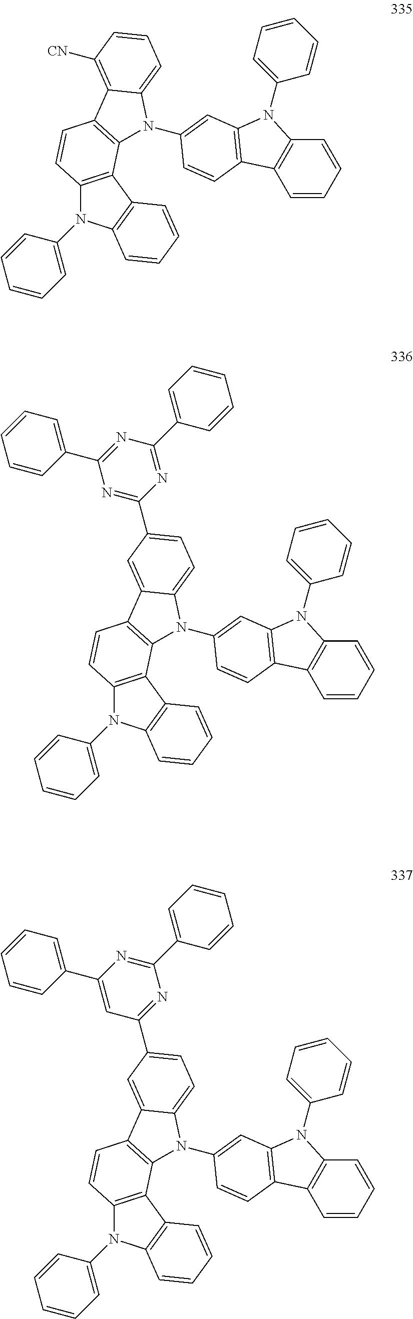

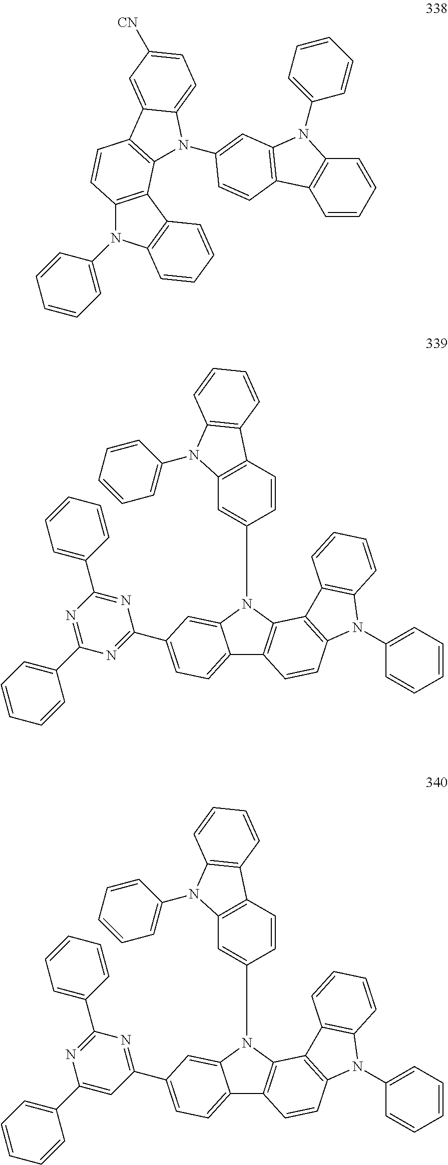

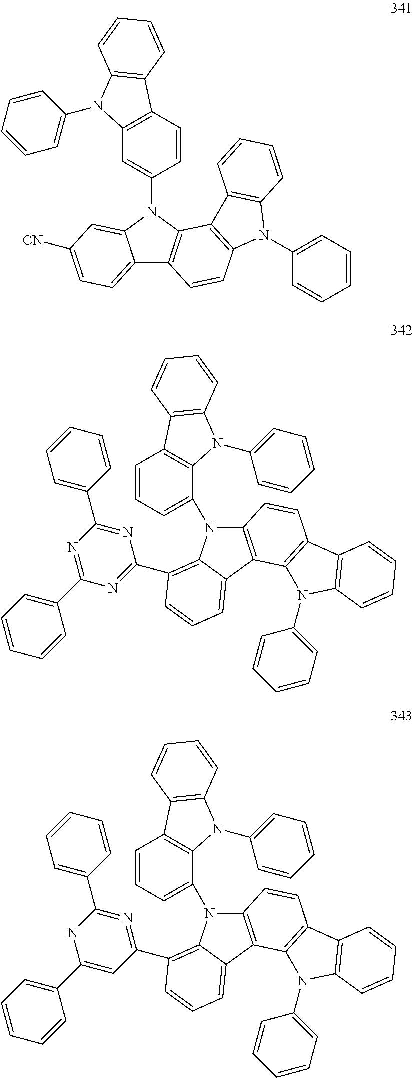

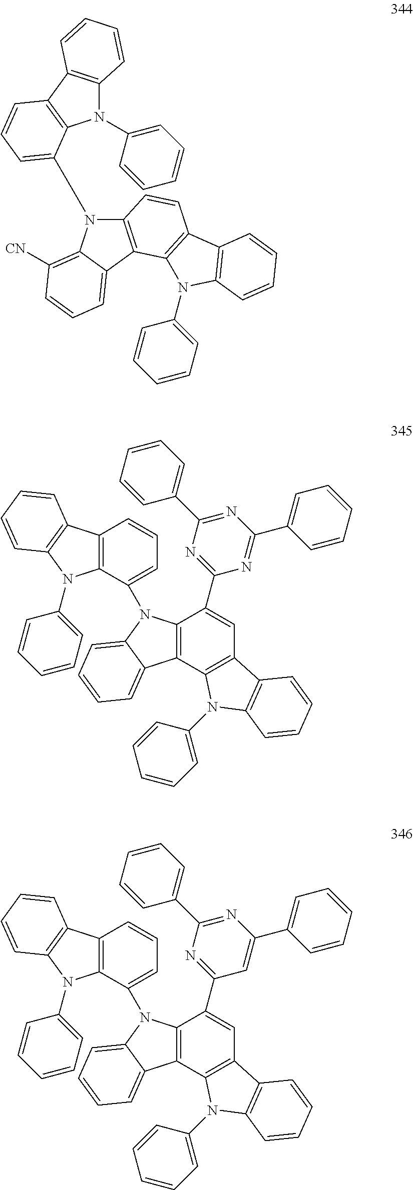

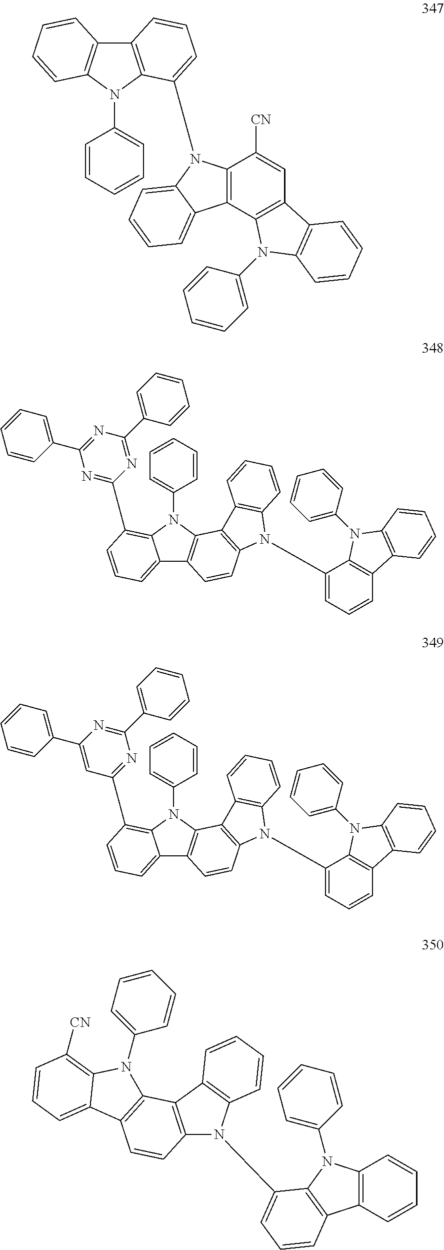 [SMILES: N#Cc1cc2c3ccccc3n(-c3ccccc3)c2c2c3ccccc3n(-c3cccc4c5ccccc5n(-c5ccccc5)c34)c12 ; c1ccc(-c2nc(-c3ccccc3)nc(-c3cccc4c5ccc6c(c7ccccc7n6-c6cccc7c8ccccc8n(-c8ccccc8)c67)c5n(-c5ccccc5)c34)n2)cc1 ; c1ccc(-c2cc(-c3cccc4c5ccc6c(c7ccccc7n6-c6cccc7c8ccccc8n(-c8ccccc8)c67)c5n(-c5ccccc5)c34)nc(-c3ccccc3)n2)cc1 ; N#Cc1cccc2c3ccc4c(c5ccccc5n4-c4cccc5c6ccccc6n(-c6ccccc6)c45)c3n(-c3ccccc3)c12]

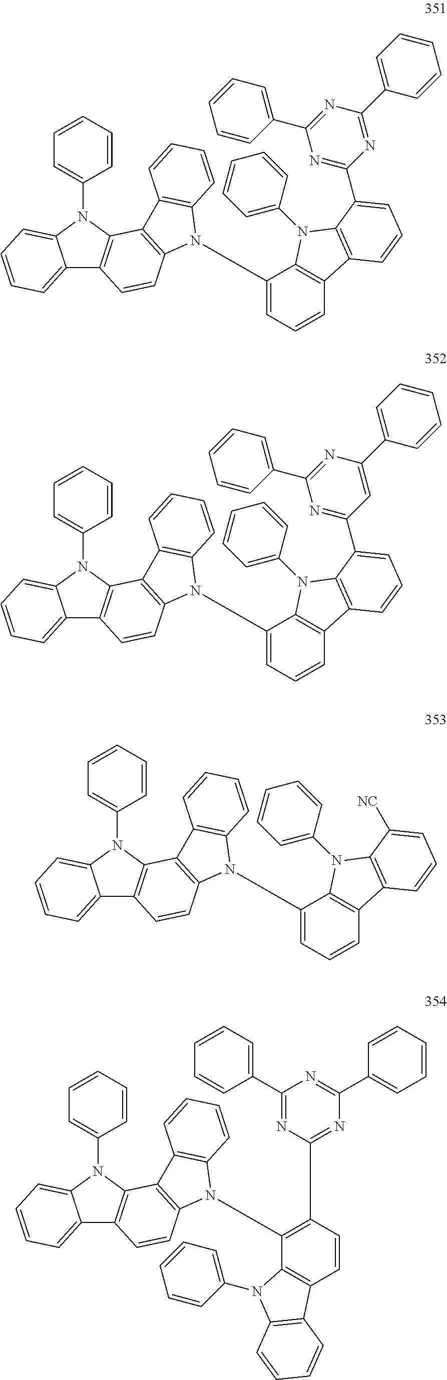

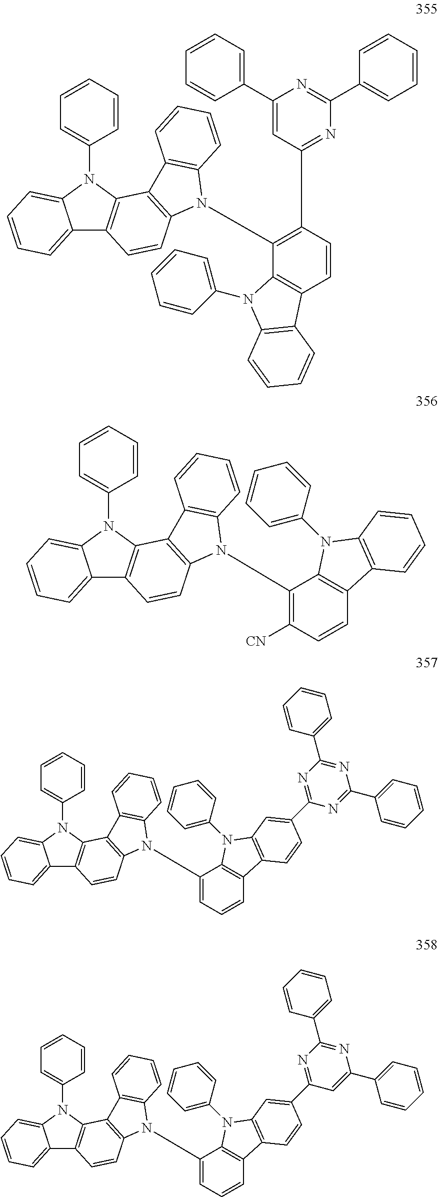

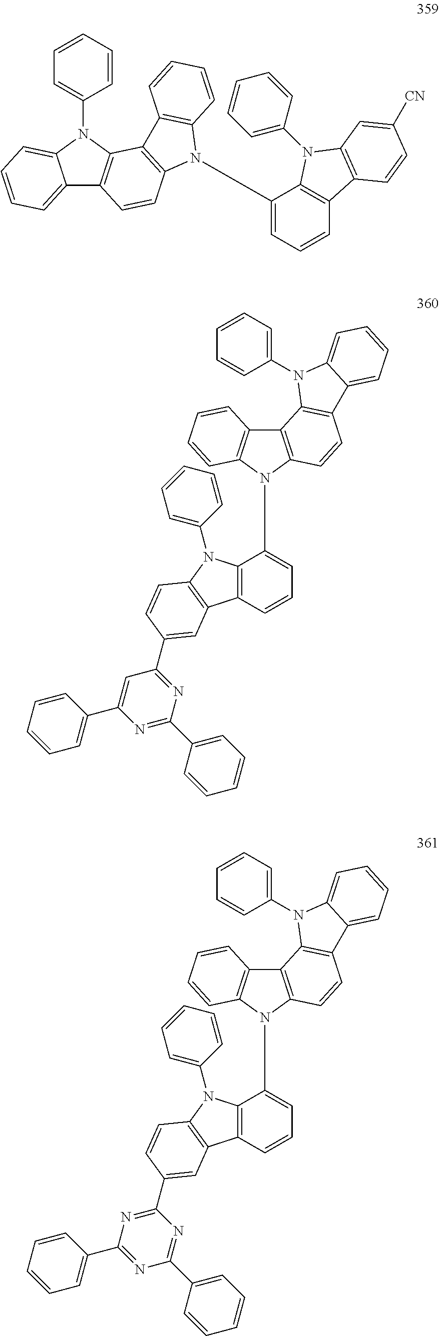 [SMILES: N#Cc1ccc2c3cccc(-n4c5ccccc5c5c4ccc4c6ccccc6n(-c6ccccc6)c45)c3n(-c3ccccc3)c2c1 ; c1ccc(-c2cc(-c3ccc4c(c3)c3cccc(-n5c6ccccc6c6c5ccc5c7ccccc7n(-c7ccccc7)c56)c3n4-c3ccccc3)nc(-c3ccccc3)n2)cc1 ; c1ccc(-c2nc(-c3ccccc3)nc(-c3ccc4c(c3)c3cccc(-n5c6ccccc6c6c5ccc5c7ccccc7n(-c7ccccc7)c56)c3n4-c3ccccc3)n2)cc1]

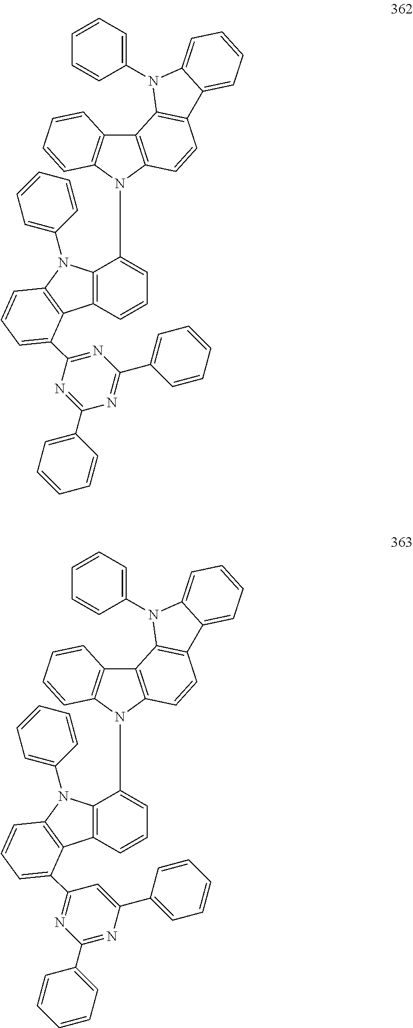 [SMILES: c1ccc(-c2nc(-c3ccccc3)nc(-c3cccc4c3c3cccc(-n5c6ccccc6c6c5ccc5c7ccccc7n(-c7ccccc7)c56)c3n4-c3ccccc3)n2)cc1 ; c1ccc(-c2cc(-c3cccc4c3c3cccc(-n5c6ccccc6c6c5ccc5c7ccccc7n(-c7ccccc7)c56)c3n4-c3ccccc3)nc(-c3ccccc3)n2)cc1]

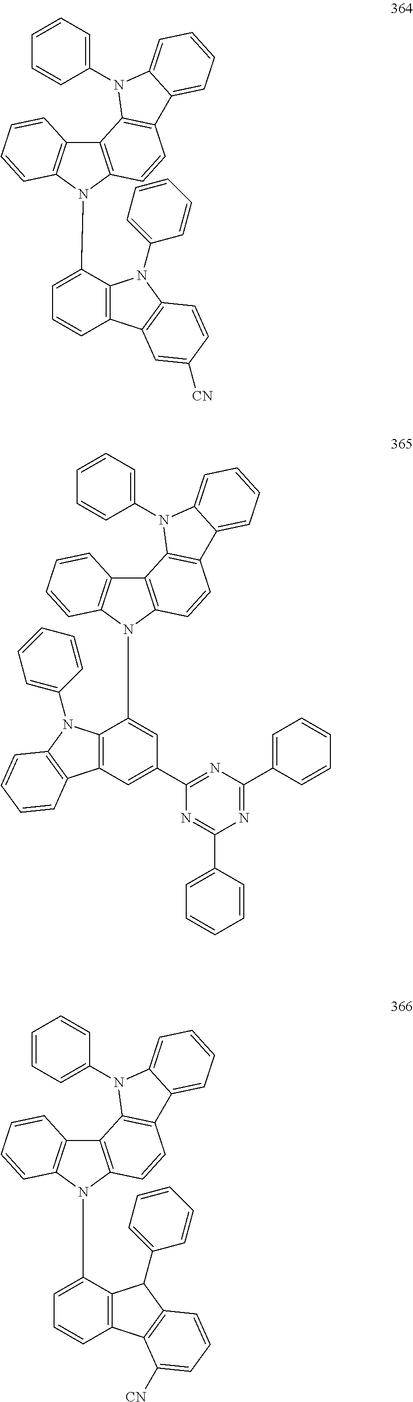

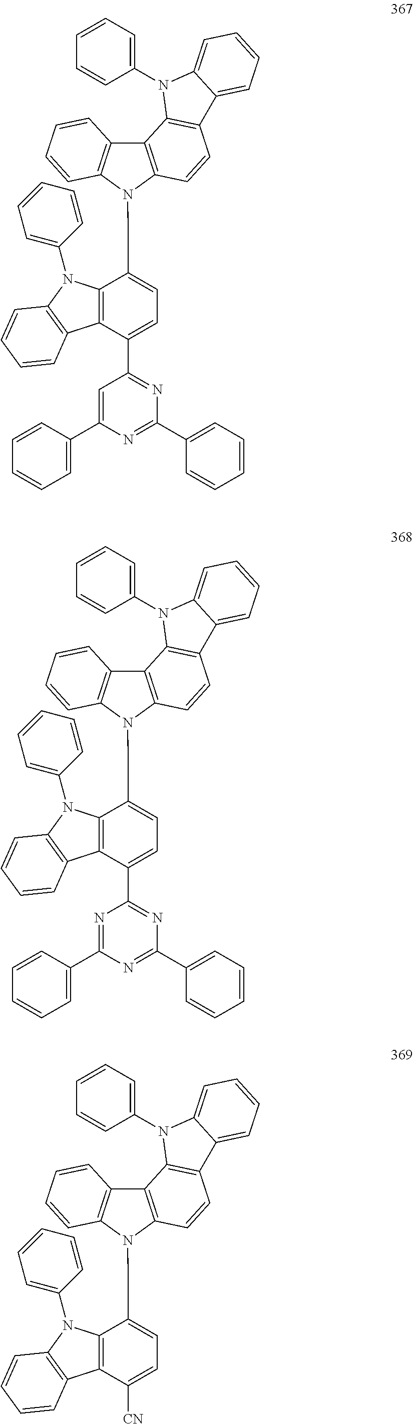 [SMILES: c1ccc(-c2cc(-c3ccc(-n4c5ccccc5c5c4ccc4c6ccccc6n(-c6ccccc6)c45)c4c3c3ccccc3n4-c3ccccc3)nc(-c3ccccc3)n2)cc1 ; c1ccc(-c2nc(-c3ccccc3)nc(-c3ccc(-n4c5ccccc5c5c4ccc4c6ccccc6n(-c6ccccc6)c45)c4c3c3ccccc3n4-c3ccccc3)n2)cc1 ; N#Cc1ccc(-n2c3ccccc3c3c2ccc2c4ccccc4n(-c4ccccc4)c23)c2c1c1ccccc1n2-c1ccccc1]

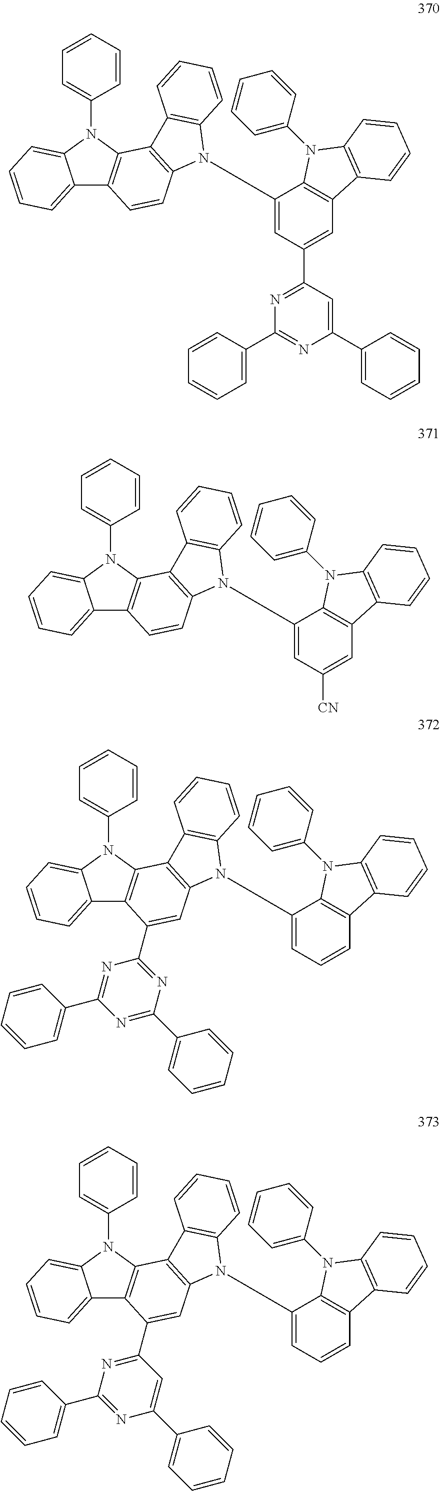 [SMILES: c1ccc(-c2cc(-c3cc(-n4c5ccccc5c5c4ccc4c6ccccc6n(-c6ccccc6)c45)c4c(c3)c3ccccc3n4-c3ccccc3)nc(-c3ccccc3)n2)cc1 ; N#Cc1cc(-n2c3ccccc3c3c2ccc2c4ccccc4n(-c4ccccc4)c23)c2c(c1)c1ccccc1n2-c1ccccc1 ; c1ccc(-c2nc(-c3ccccc3)nc(-c3cc4c(c5ccccc5n4-c4cccc5c6ccccc6n(-c6ccccc6)c45)c4c3c3ccccc3n4-c3ccccc3)n2)cc1 ; c1ccc(-c2cc(-c3cc4c(c5ccccc5n4-c4cccc5c6ccccc6n(-c6ccccc6)c45)c4c3c3ccccc3n4-c3ccccc3)nc(-c3ccccc3)n2)cc1]

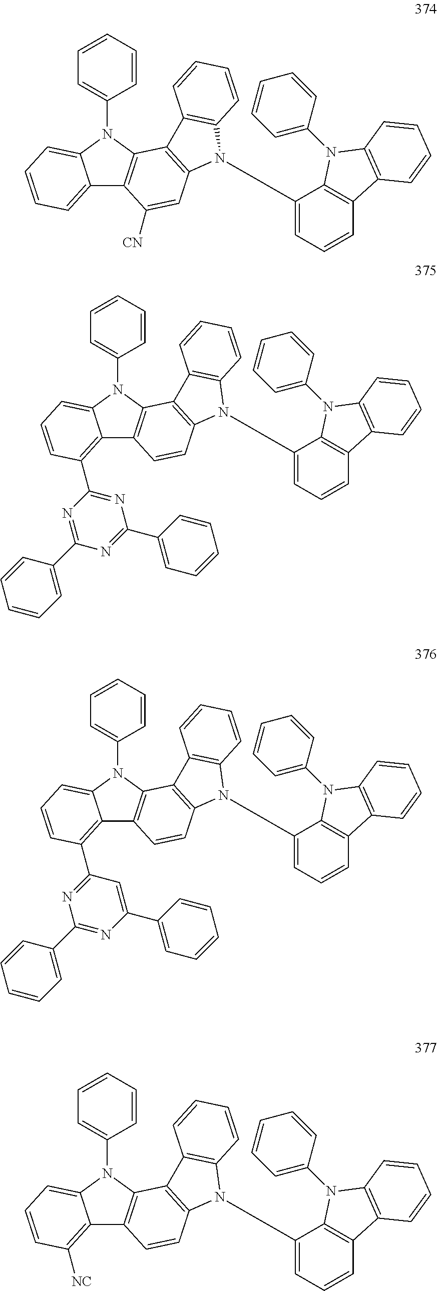

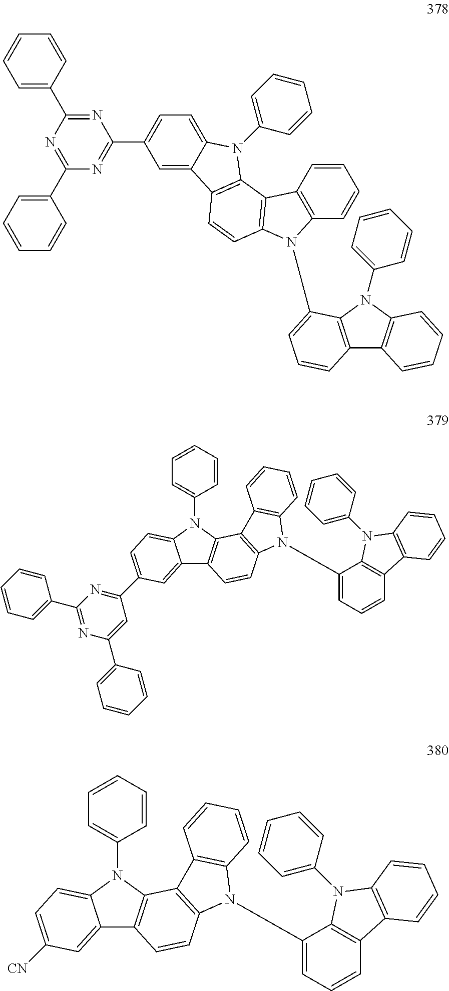

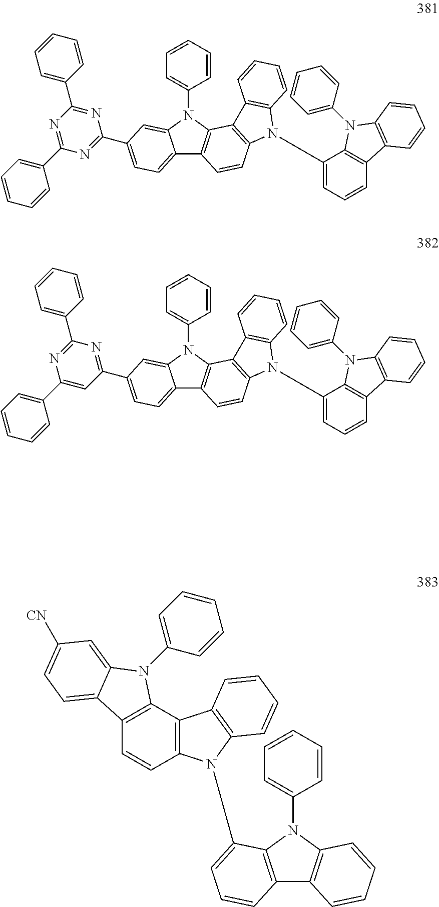

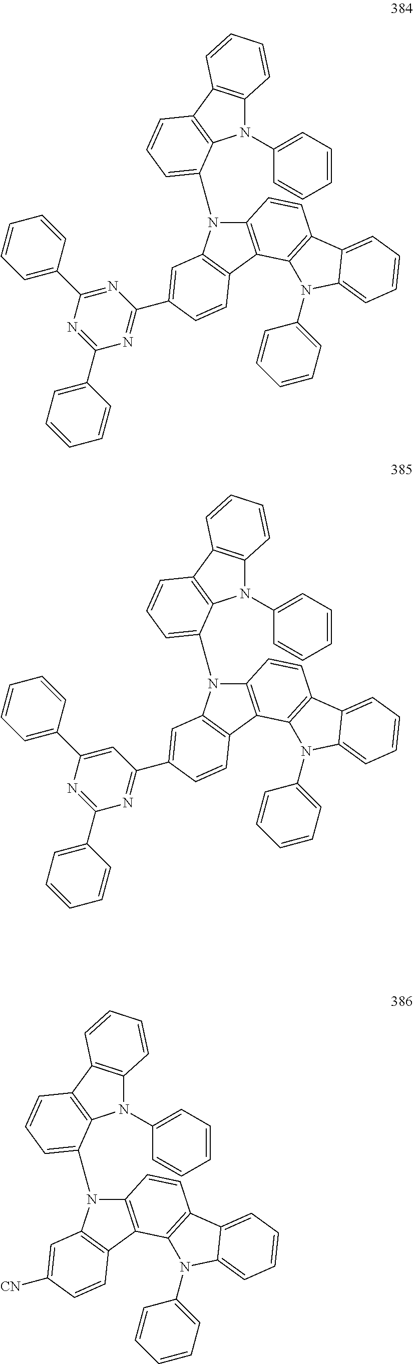

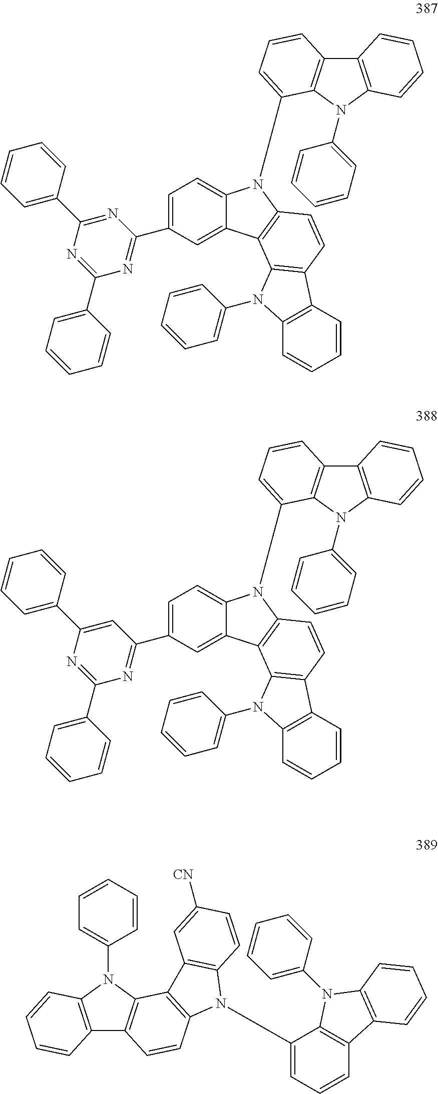

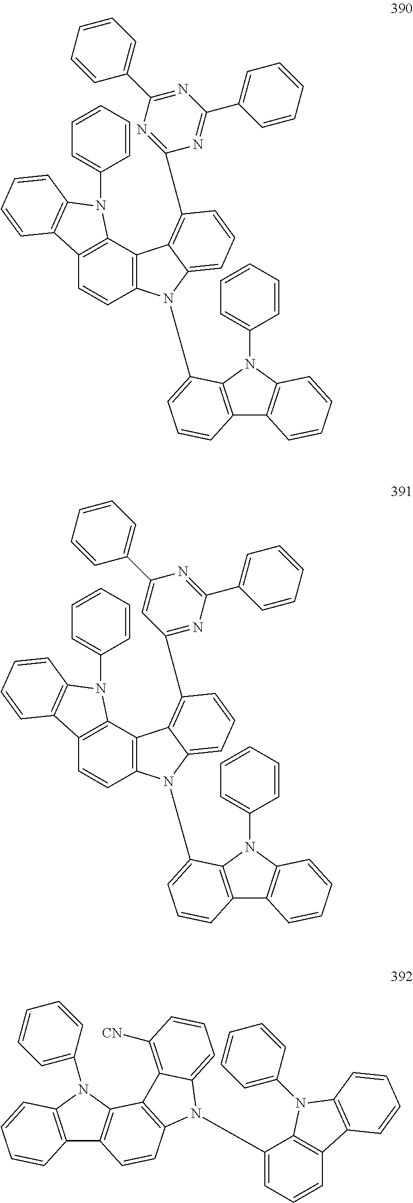

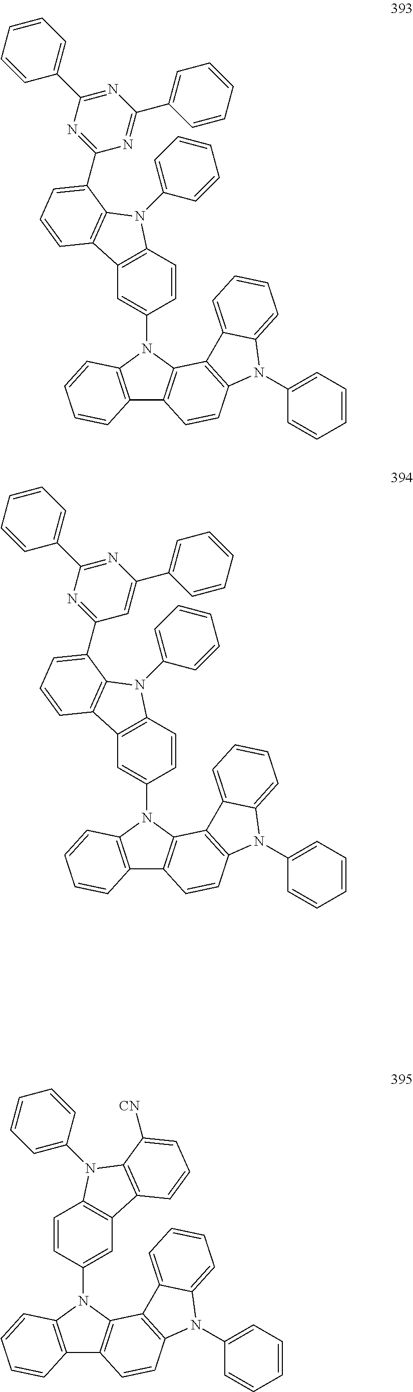

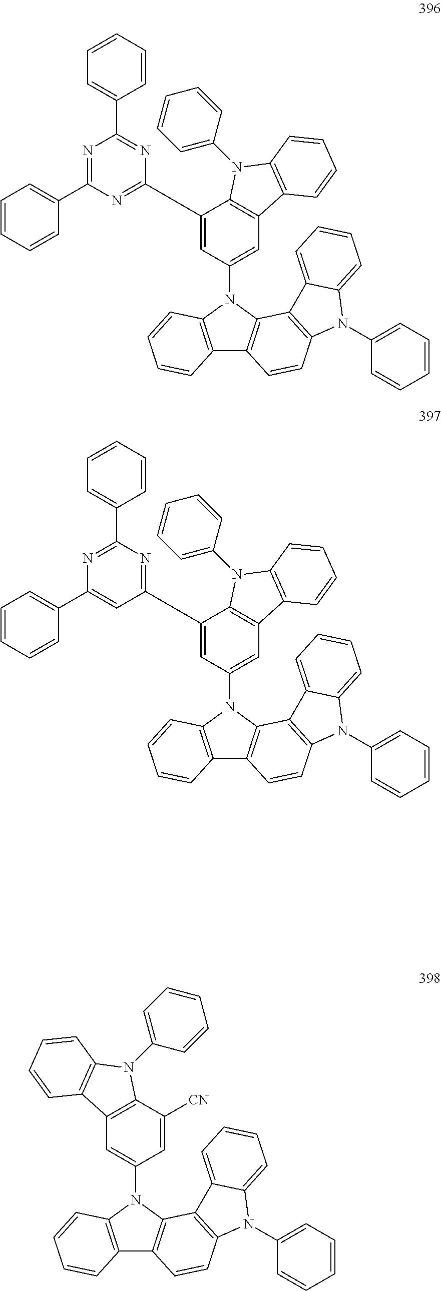 [SMILES: c1ccc(-c2nc(-c3ccccc3)nc(-c3cc(-n4c5ccccc5c5ccc6c(c7ccccc7n6-c6ccccc6)c54)cc4c5ccccc5n(-c5ccccc5)c34)n2)cc1 ; c1ccc(-c2cc(-c3cc(-n4c5ccccc5c5ccc6c(c7ccccc7n6-c6ccccc6)c54)cc4c5ccccc5n(-c5ccccc5)c34)nc(-c3ccccc3)n2)cc1 ; N#Cc1cc(-n2c3ccccc3c3ccc4c(c5ccccc5n4-c4ccccc4)c32)cc2c3ccccc3n(-c3ccccc3)c12]

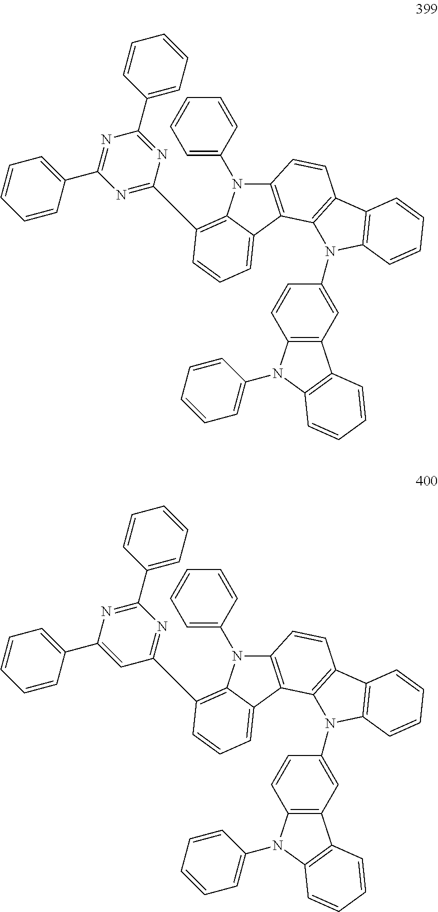 [SMILES: c1ccc(-c2nc(-c3ccccc3)nc(-c3cccc4c5c(ccc6c7ccccc7n(-c7ccc8c(c7)c7ccccc7n8-c7ccccc7)c65)n(-c5ccccc5)c34)n2)cc1 ; c1ccc(-c2cc(-c3cccc4c5c(ccc6c7ccccc7n(-c7ccc8c(c7)c7ccccc7n8-c7ccccc7)c65)n(-c5ccccc5)c34)nc(-c3ccccc3)n2)cc1]

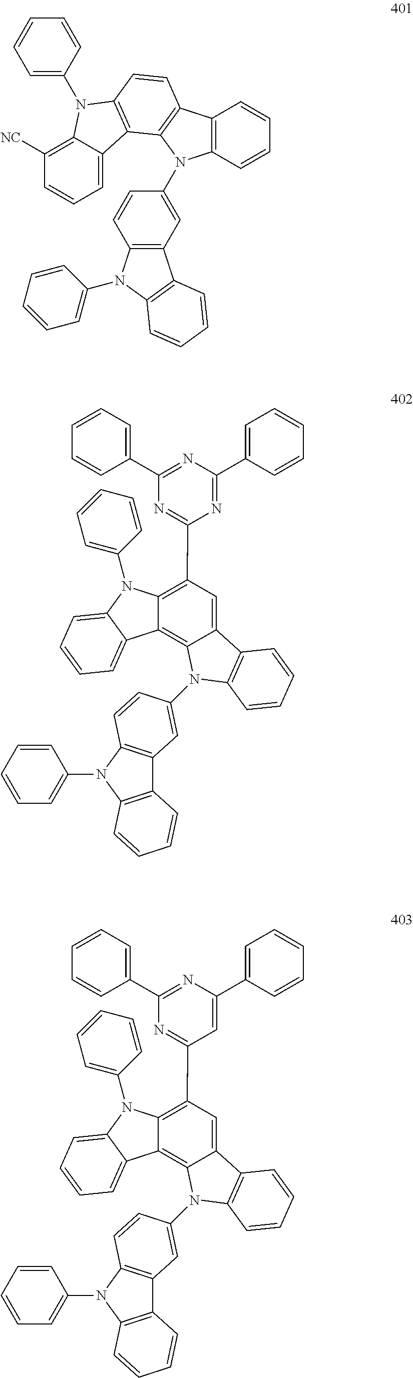 [SMILES: N#Cc1cccc2c3c(ccc4c5ccccc5n(-c5ccc6c(c5)c5ccccc5n6-c5ccccc5)c43)n(-c3ccccc3)c12 ; c1ccc(-c2nc(-c3ccccc3)nc(-c3cc4c5ccccc5n(-c5ccc6c(c5)c5ccccc5n6-c5ccccc5)c4c4c5ccccc5n(-c5ccccc5)c34)n2)cc1 ; c1ccc(-c2cc(-c3cc4c5ccccc5n(-c5ccc6c(c5)c5ccccc5n6-c5ccccc5)c4c4c5ccccc5n(-c5ccccc5)c34)nc(-c3ccccc3)n2)cc1]

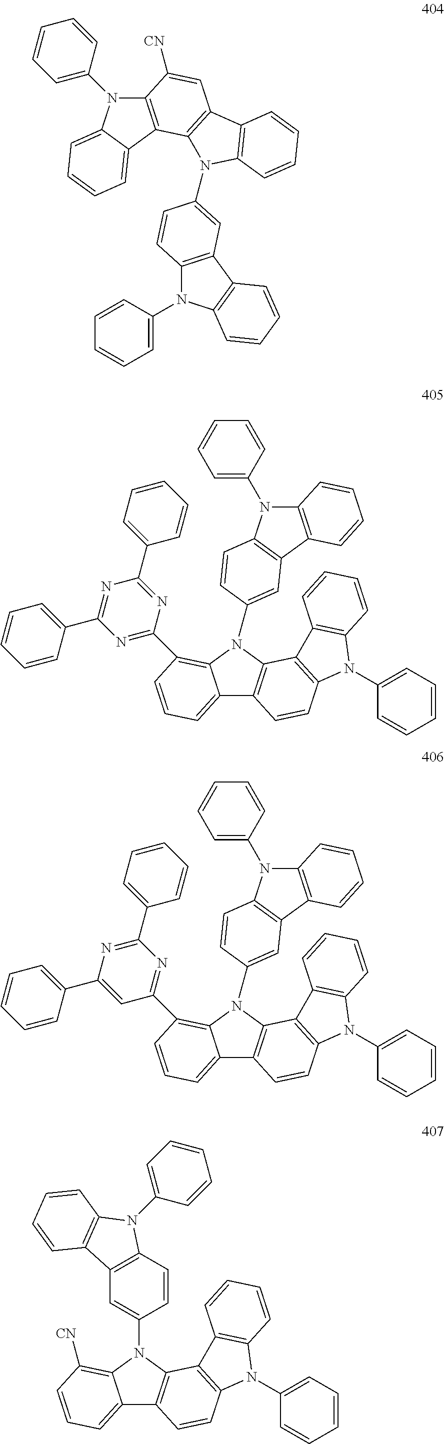

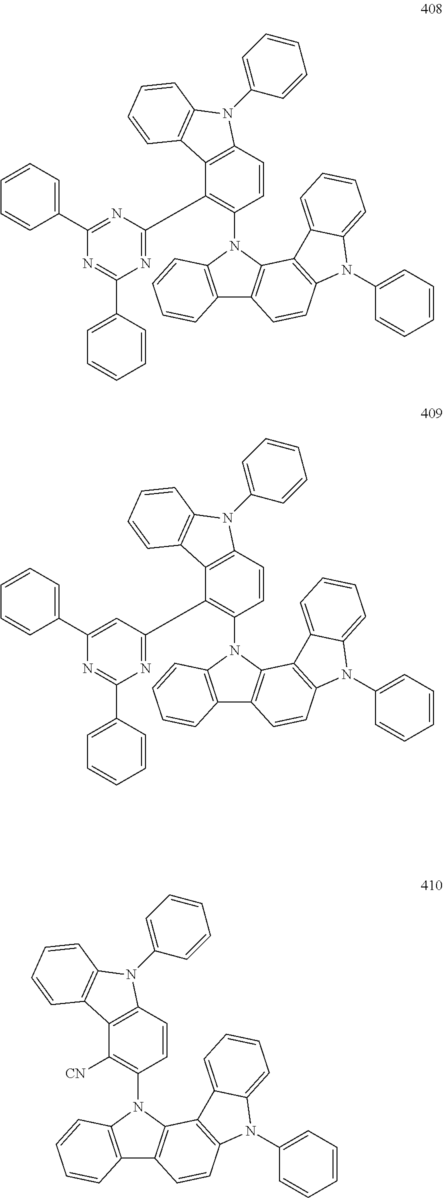

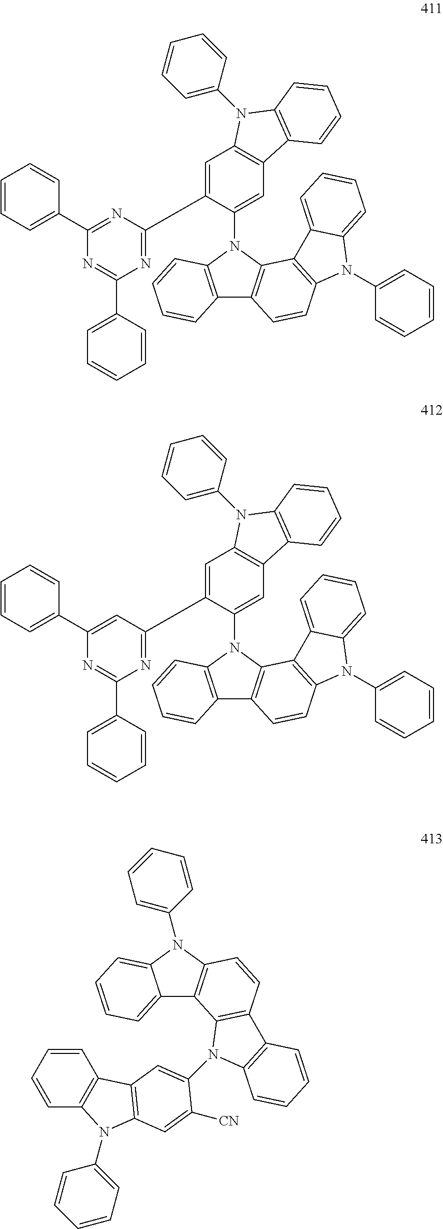 [SMILES: c1ccc(-c2nc(-c3ccccc3)nc(-c3cc4c(cc3-n3c5ccccc5c5ccc6c(c7ccccc7n6-c6ccccc6)c53)c3ccccc3n4-c3ccccc3)n2)cc1 ; c1ccc(-c2cc(-c3cc4c(cc3-n3c5ccccc5c5ccc6c(c7ccccc7n6-c6ccccc6)c53)c3ccccc3n4-c3ccccc3)nc(-c3ccccc3)n2)cc1 ; N#Cc1cc2c(cc1-n1c3ccccc3c3ccc4c(c5ccccc5n4-c4ccccc4)c31)c1ccccc1n2-c1ccccc1]

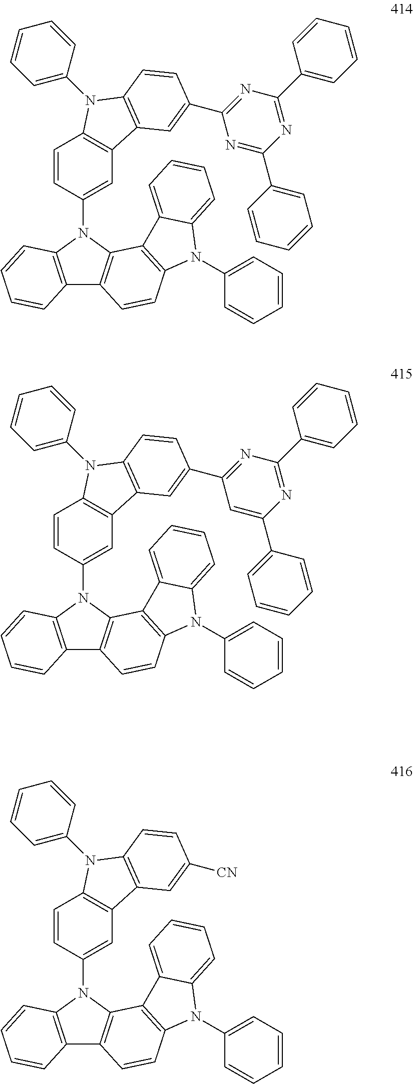 [SMILES: c1ccc(-c2nc(-c3ccccc3)nc(-c3ccc4c(c3)c3cc(-n5c6ccccc6c6ccc7c(c8ccccc8n7-c7ccccc7)c65)ccc3n4-c3ccccc3)n2)cc1 ; c1ccc(-c2cc(-c3ccc4c(c3)c3cc(-n5c6ccccc6c6ccc7c(c8ccccc8n7-c7ccccc7)c65)ccc3n4-c3ccccc3)nc(-c3ccccc3)n2)cc1 ; N#Cc1ccc2c(c1)c1cc(-n3c4ccccc4c4ccc5c(c6ccccc6n5-c5ccccc5)c43)ccc1n2-c1ccccc1]

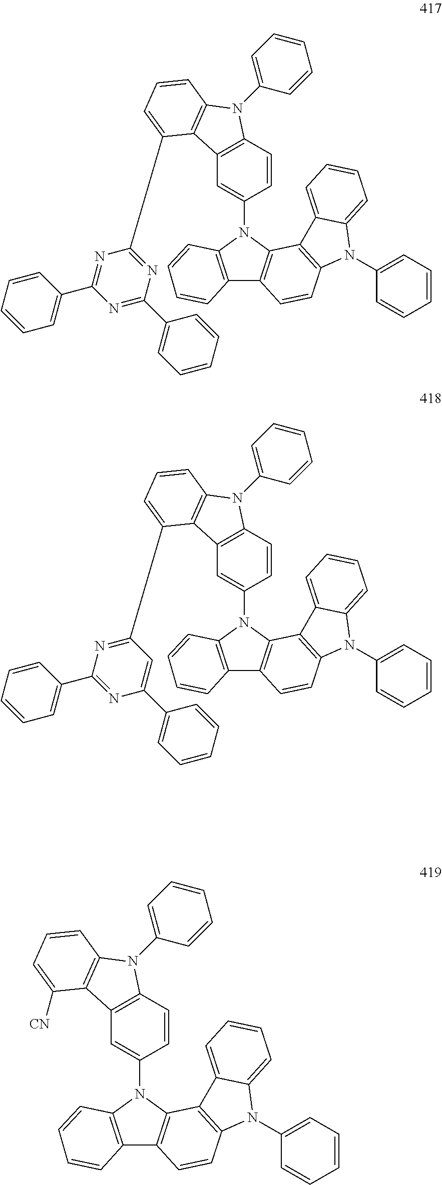

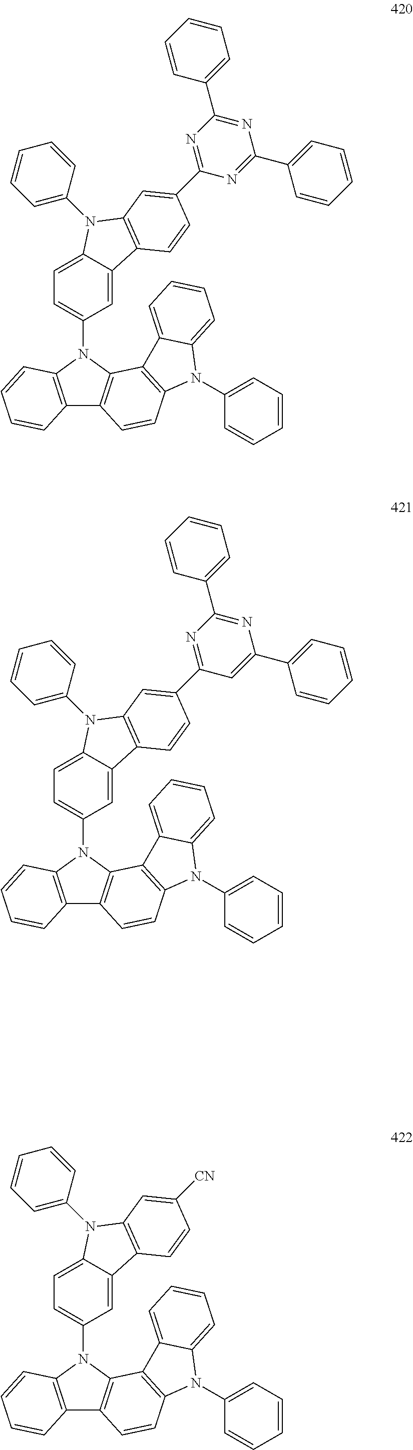 [SMILES: c1ccc(-c2nc(-c3ccccc3)nc(-c3ccc4c5cc(-n6c7ccccc7c7ccc8c(c9ccccc9n8-c8ccccc8)c76)ccc5n(-c5ccccc5)c4c3)n2)cc1 ; c1ccc(-c2cc(-c3ccc4c5cc(-n6c7ccccc7c7ccc8c(c9ccccc9n8-c8ccccc8)c76)ccc5n(-c5ccccc5)c4c3)nc(-c3ccccc3)n2)cc1 ; N#Cc1ccc2c3cc(-n4c5ccccc5c5ccc6c(c7ccccc7n6-c6ccccc6)c54)ccc3n(-c3ccccc3)c2c1]

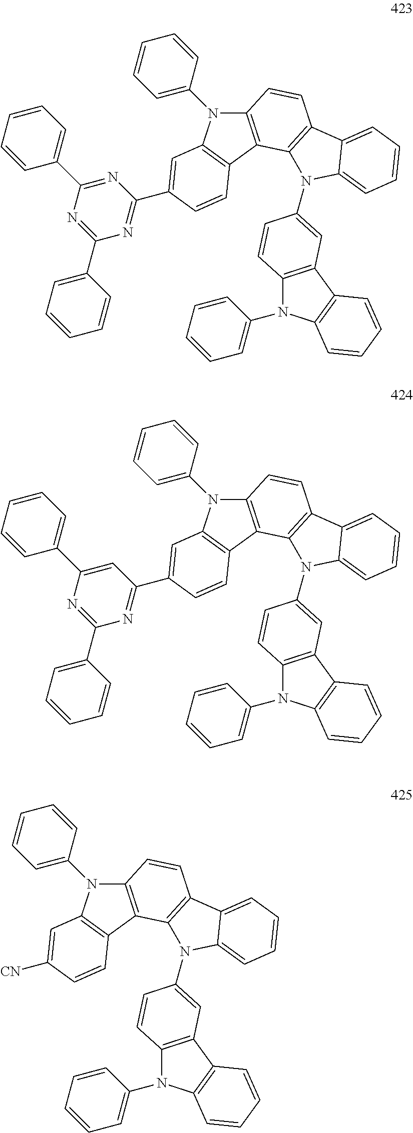

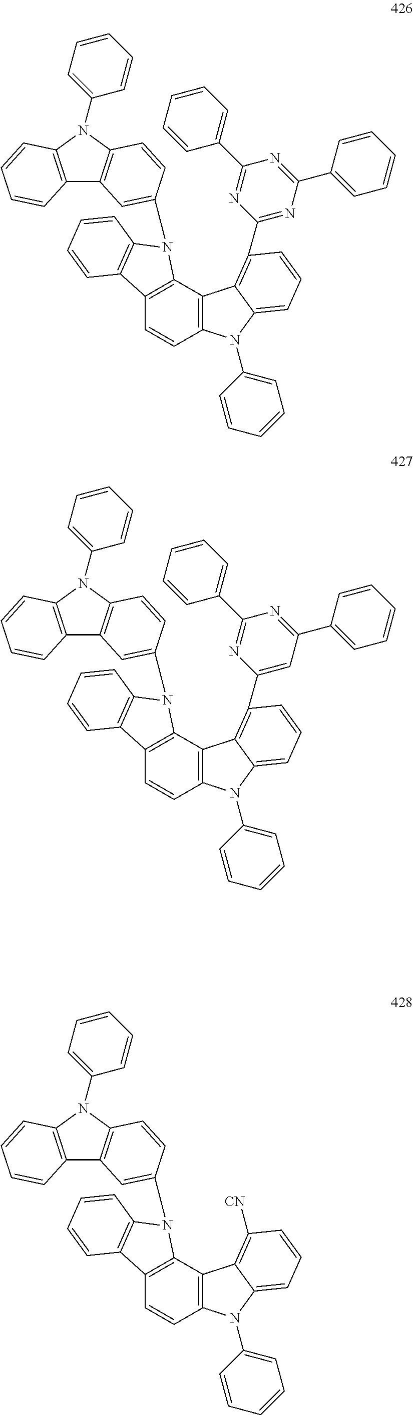

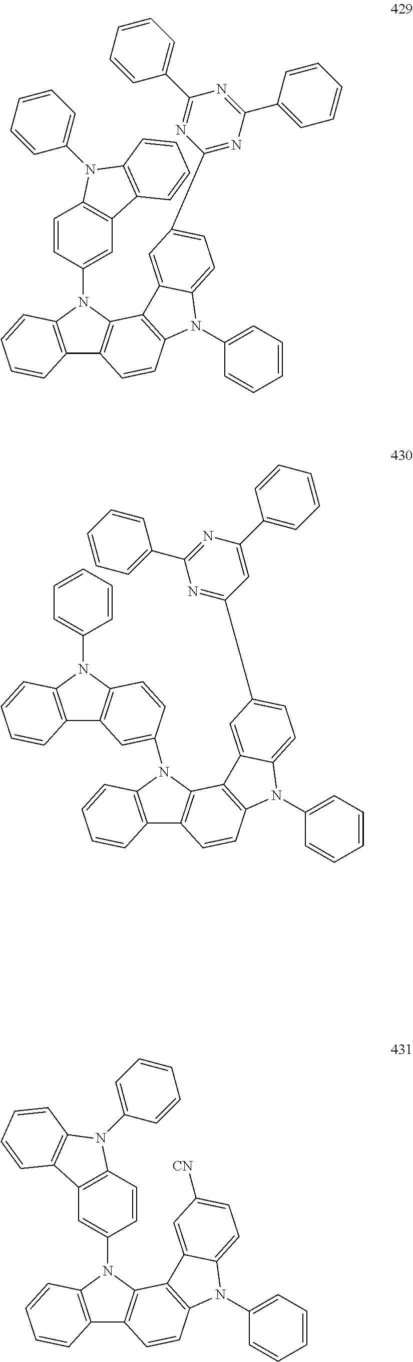

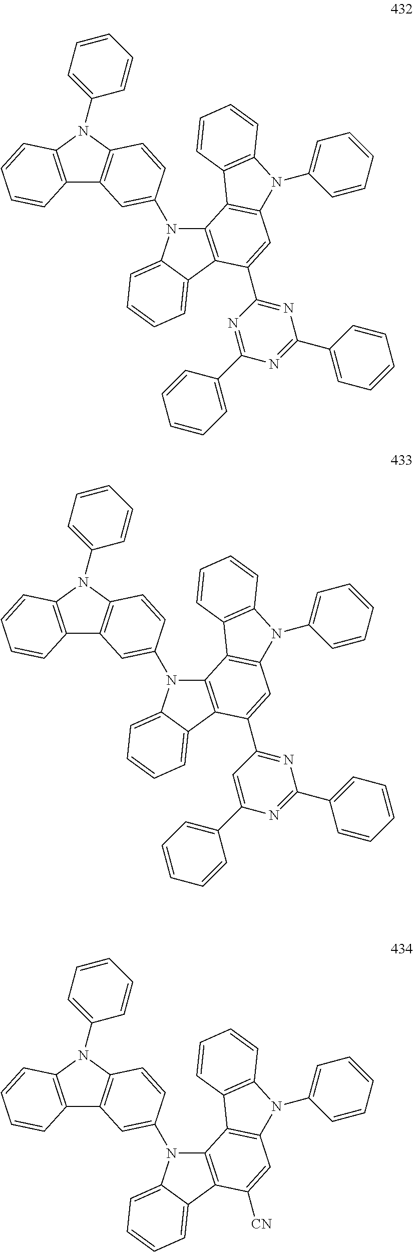 [SMILES: c1ccc(-c2nc(-c3ccccc3)nc(-c3cc4c(c5ccccc5n4-c4ccccc4)c4c3c3ccccc3n4-c3ccc4c(c3)c3ccccc3n4-c3ccccc3)n2)cc1 ; c1ccc(-c2cc(-c3cc4c(c5ccccc5n4-c4ccccc4)c4c3c3ccccc3n4-c3ccc4c(c3)c3ccccc3n4-c3ccccc3)nc(-c3ccccc3)n2)cc1 ; N#Cc1cc2c(c3ccccc3n2-c2ccccc2)c2c1c1ccccc1n2-c1ccc2c(c1)c1ccccc1n2-c1ccccc1]

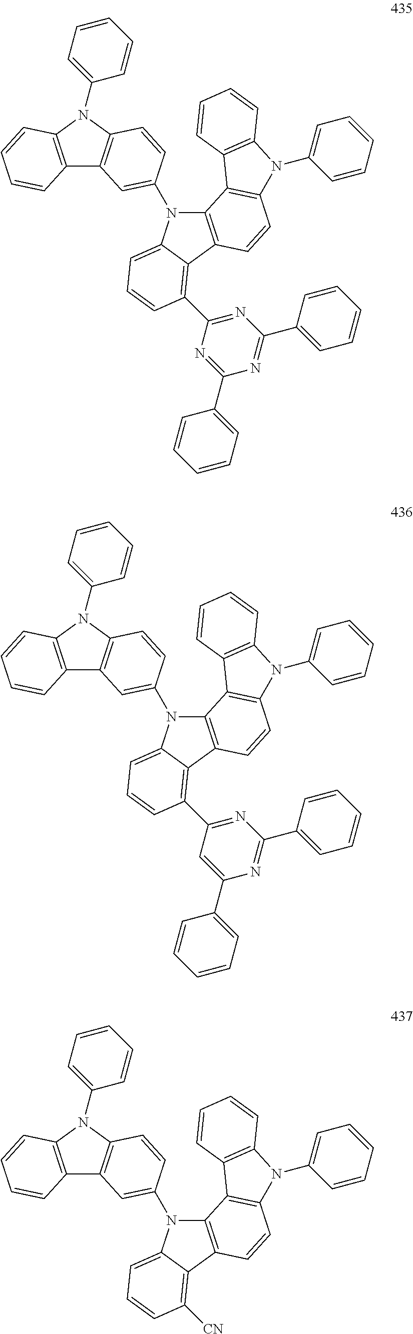 [SMILES: c1ccc(-c2nc(-c3ccccc3)nc(-c3cccc4c3c3ccc5c(c6ccccc6n5-c5ccccc5)c3n4-c3ccc4c(c3)c3ccccc3n4-c3ccccc3)n2)cc1 ; c1ccc(-c2cc(-c3cccc4c3c3ccc5c(c6ccccc6n5-c5ccccc5)c3n4-c3ccc4c(c3)c3ccccc3n4-c3ccccc3)nc(-c3ccccc3)n2)cc1 ; N#Cc1cccc2c1c1ccc3c(c4ccccc4n3-c3ccccc3)c1n2-c1ccc2c(c1)c1ccccc1n2-c1ccccc1]

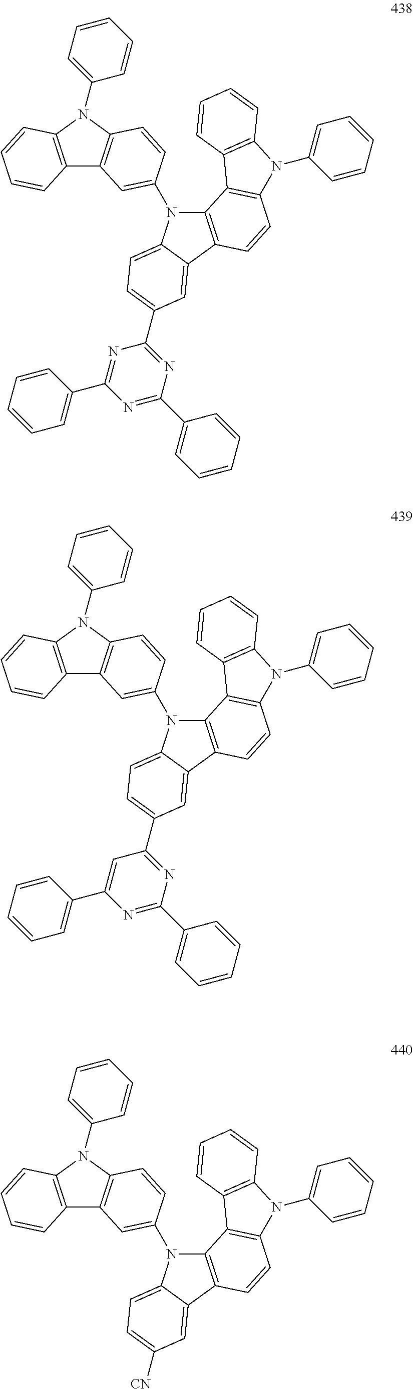

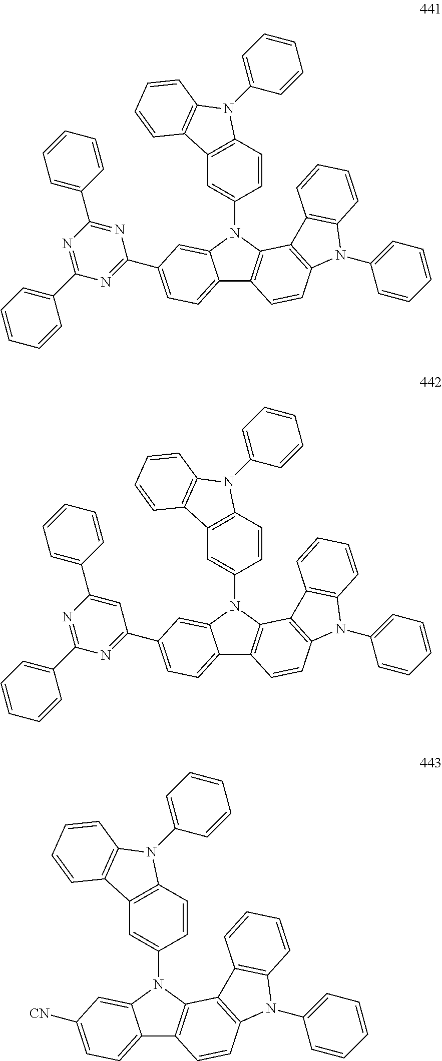

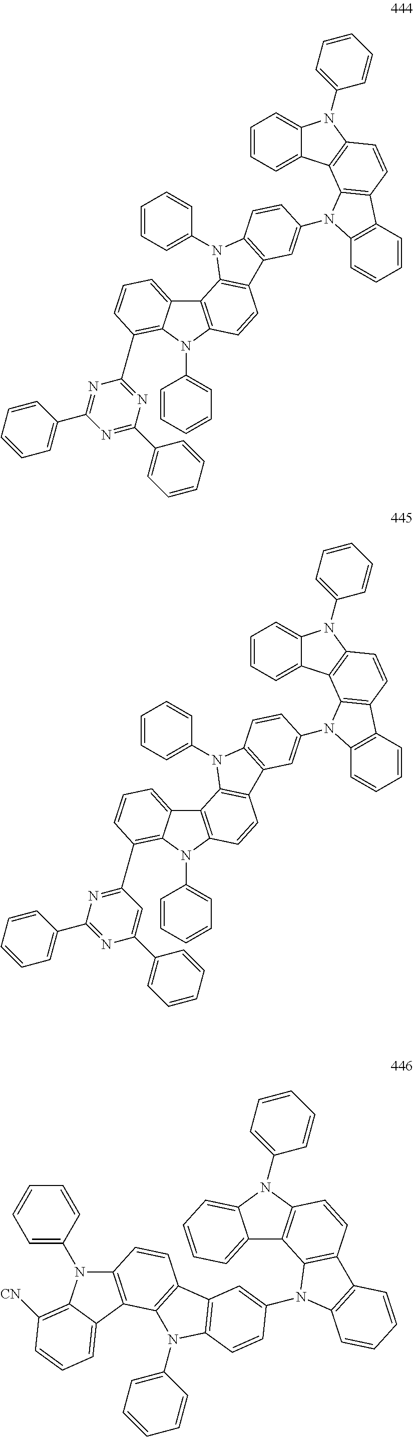

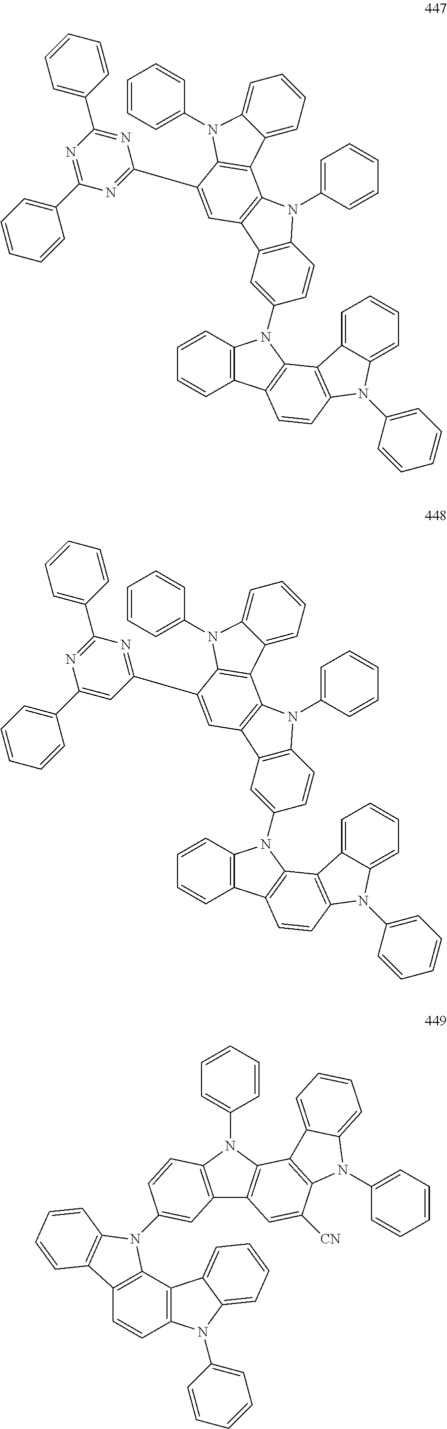 [SMILES: c1ccc(-c2nc(-c3ccccc3)nc(-c3cc4c5cc(-n6c7ccccc7c7ccc8c(c9ccccc9n8-c8ccccc8)c76)ccc5n(-c5ccccc5)c4c4c5ccccc5n(-c5ccccc5)c34)n2)cc1 ; c1ccc(-c2cc(-c3cc4c5cc(-n6c7ccccc7c7ccc8c(c9ccccc9n8-c8ccccc8)c76)ccc5n(-c5ccccc5)c4c4c5ccccc5n(-c5ccccc5)c34)nc(-c3ccccc3)n2)cc1 ; N#Cc1cc2c3cc(-n4c5ccccc5c5ccc6c(c7ccccc7n6-c6ccccc6)c54)ccc3n(-c3ccccc3)c2c2c3ccccc3n(-c3ccccc3)c12]

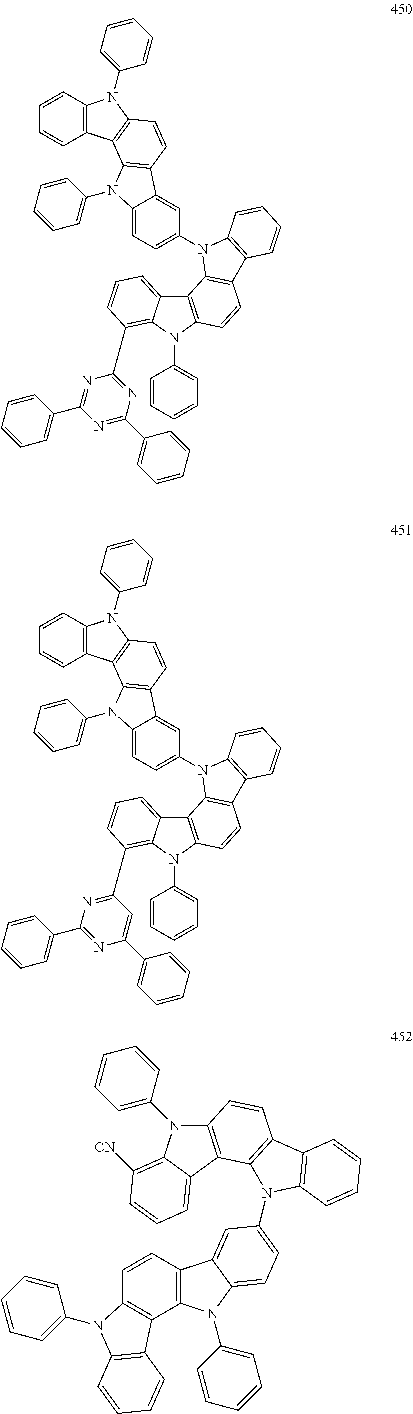

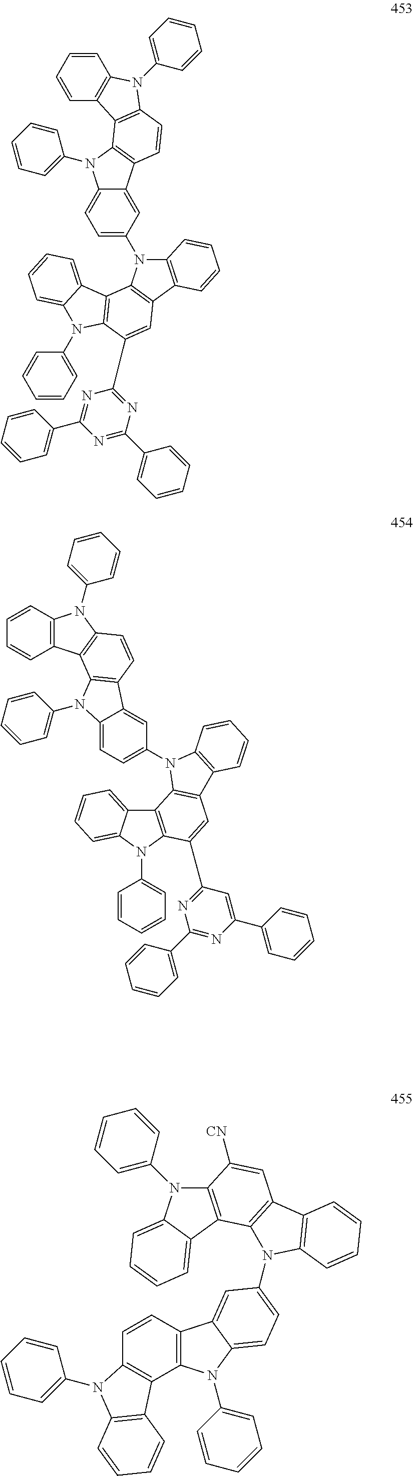

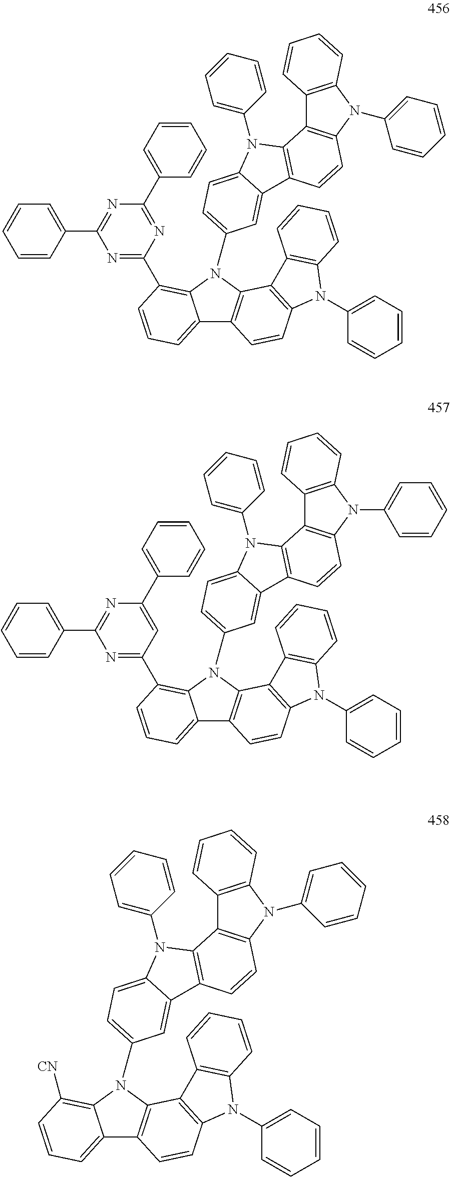

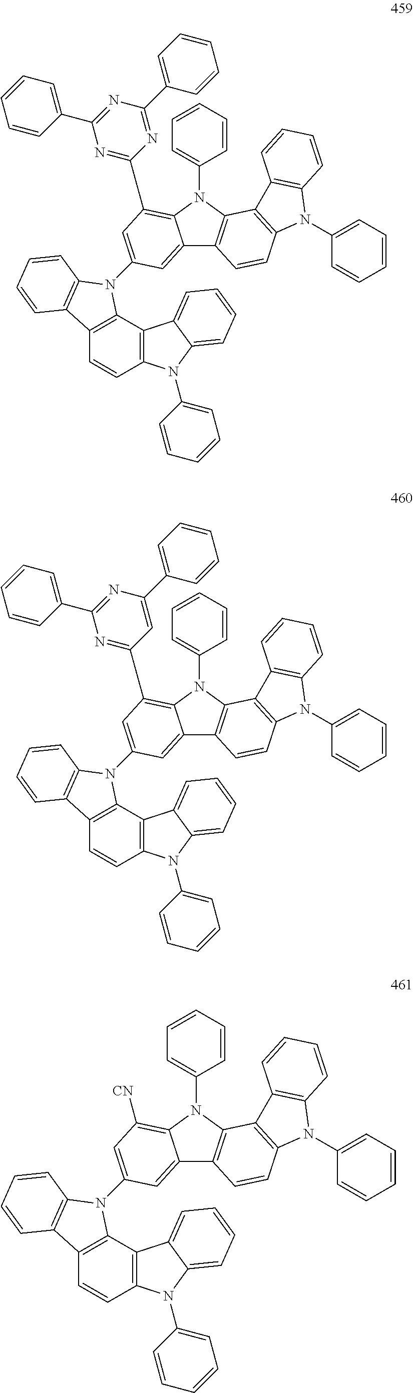

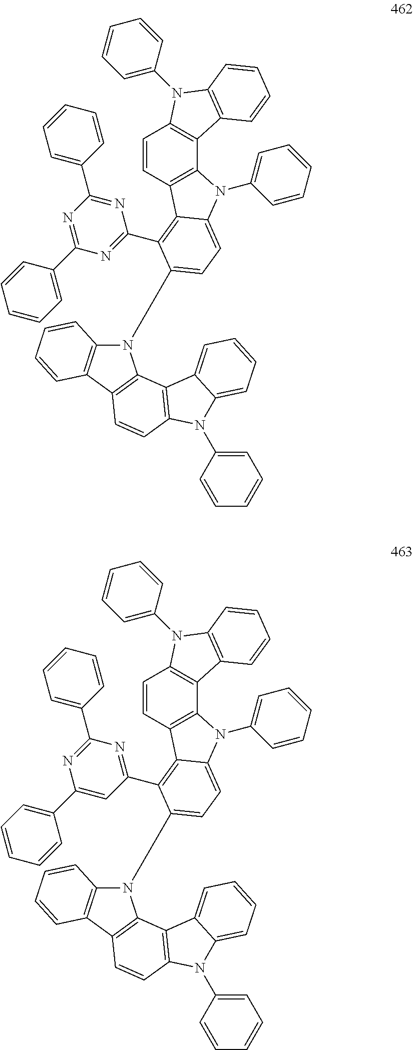 [SMILES: c1ccc(-c2nc(-c3ccccc3)nc(-c3c(-n4c5ccccc5c5ccc6c(c7ccccc7n6-c6ccccc6)c54)ccc4c3c3ccc5c(c6ccccc6n5-c5ccccc5)c3n4-c3ccccc3)n2)cc1 ; c1ccc(-c2cc(-c3c(-n4c5ccccc5c5ccc6c(c7ccccc7n6-c6ccccc6)c54)ccc4c3c3ccc5c(c6ccccc6n5-c5ccccc5)c3n4-c3ccccc3)nc(-c3ccccc3)n2)cc1]

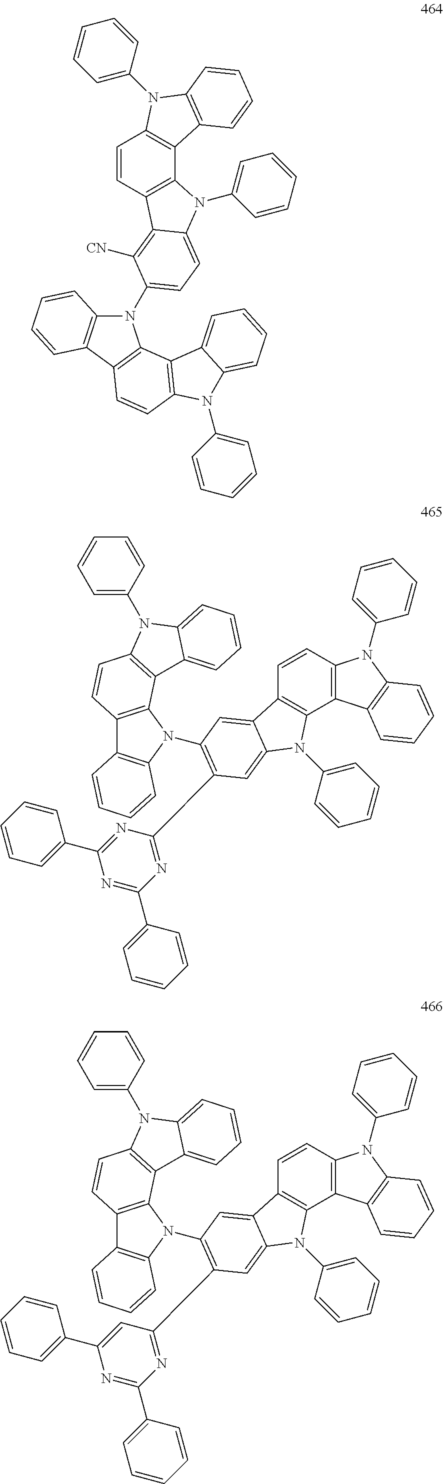

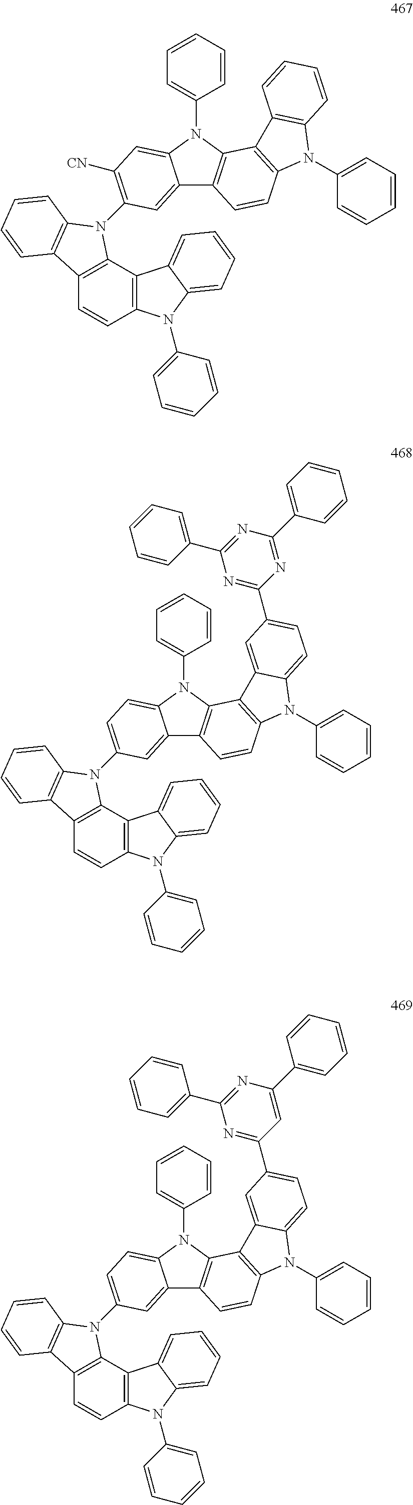

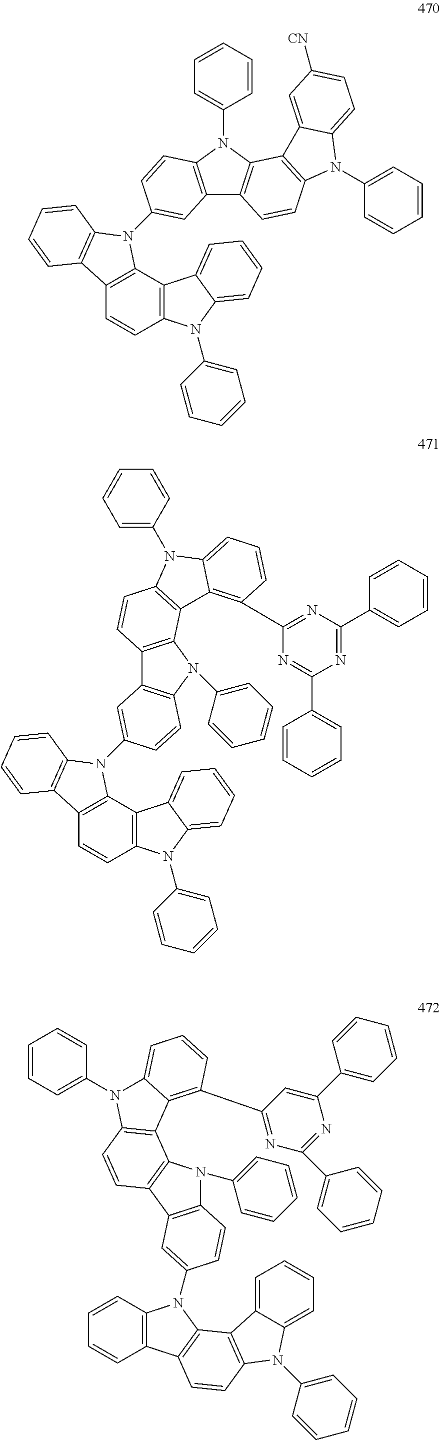

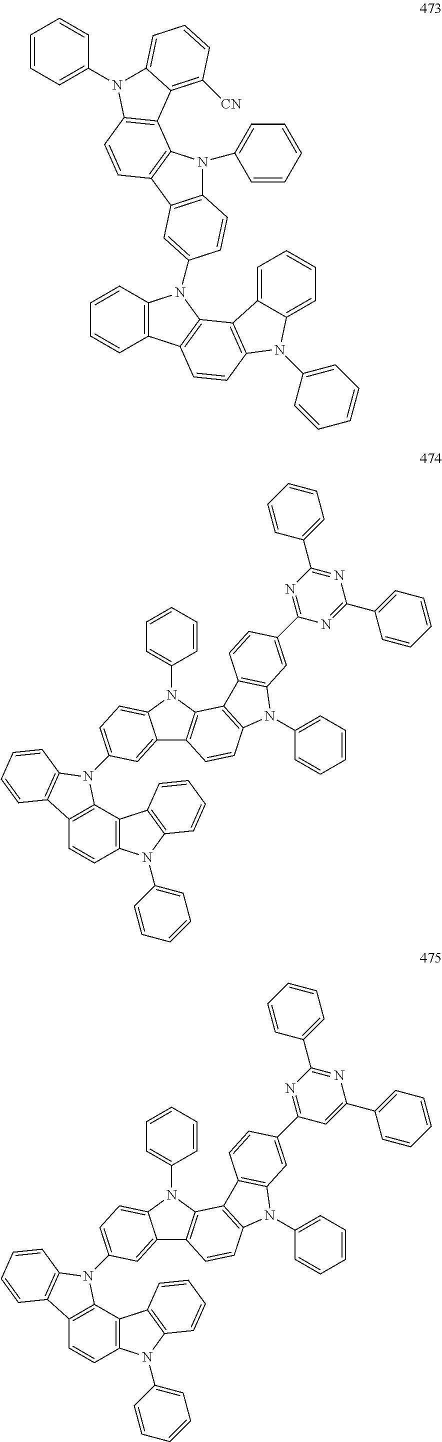 [SMILES: N#Cc1cccc2c1c1c(ccc3c4cc(-n5c6ccccc6c6ccc7c(c8ccccc8n7-c7ccccc7)c65)ccc4n(-c4ccccc4)c31)n2-c1ccccc1 ; c1ccc(-c2nc(-c3ccccc3)nc(-c3ccc4c5c(ccc6c7cc(-n8c9ccccc9c9ccc%10c(c%11ccccc%11n%10-c%10ccccc%10)c98)ccc7n(-c7ccccc7)c65)n(-c5ccccc5)c4c3)n2)cc1 ; c1ccc(-c2cc(-c3ccc4c5c(ccc6c7cc(-n8c9ccccc9c9ccc%10c(c%11ccccc%11n%10-c%10ccccc%10)c98)ccc7n(-c7ccccc7)c65)n(-c5ccccc5)c4c3)nc(-c3ccccc3)n2)cc1]

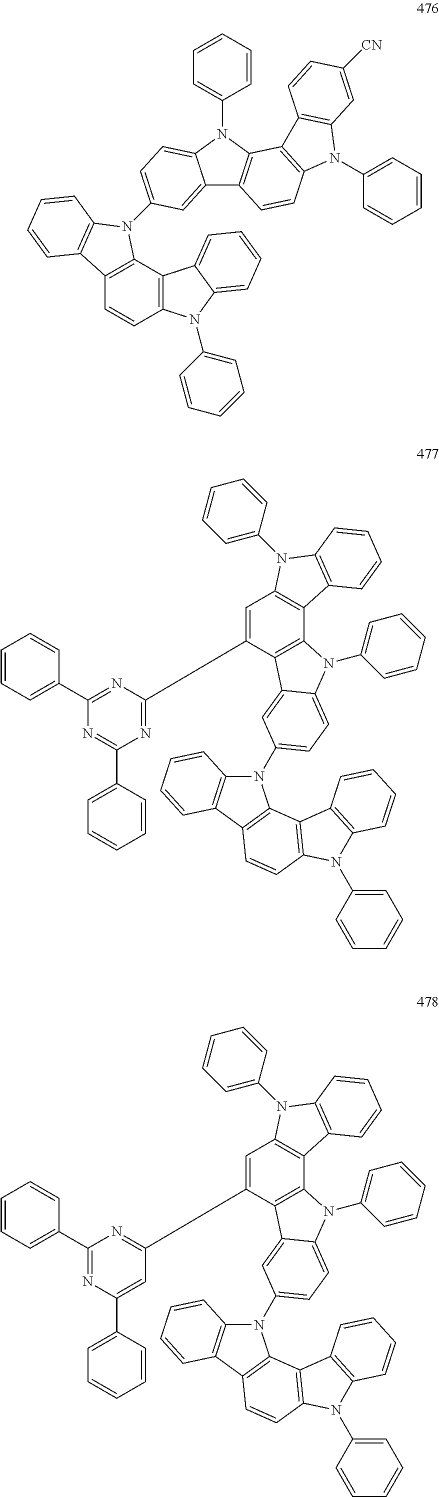 [SMILES: N#Cc1ccc2c3c(ccc4c5cc(-n6c7ccccc7c7ccc8c(c9ccccc9n8-c8ccccc8)c76)ccc5n(-c5ccccc5)c43)n(-c3ccccc3)c2c1 ; c1ccc(-c2nc(-c3ccccc3)nc(-c3cc4c(c5ccccc5n4-c4ccccc4)c4c3c3cc(-n5c6ccccc6c6ccc7c(c8ccccc8n7-c7ccccc7)c65)ccc3n4-c3ccccc3)n2)cc1 ; c1ccc(-c2cc(-c3cc4c(c5ccccc5n4-c4ccccc4)c4c3c3cc(-n5c6ccccc6c6ccc7c(c8ccccc8n7-c7ccccc7)c65)ccc3n4-c3ccccc3)nc(-c3ccccc3)n2)cc1]

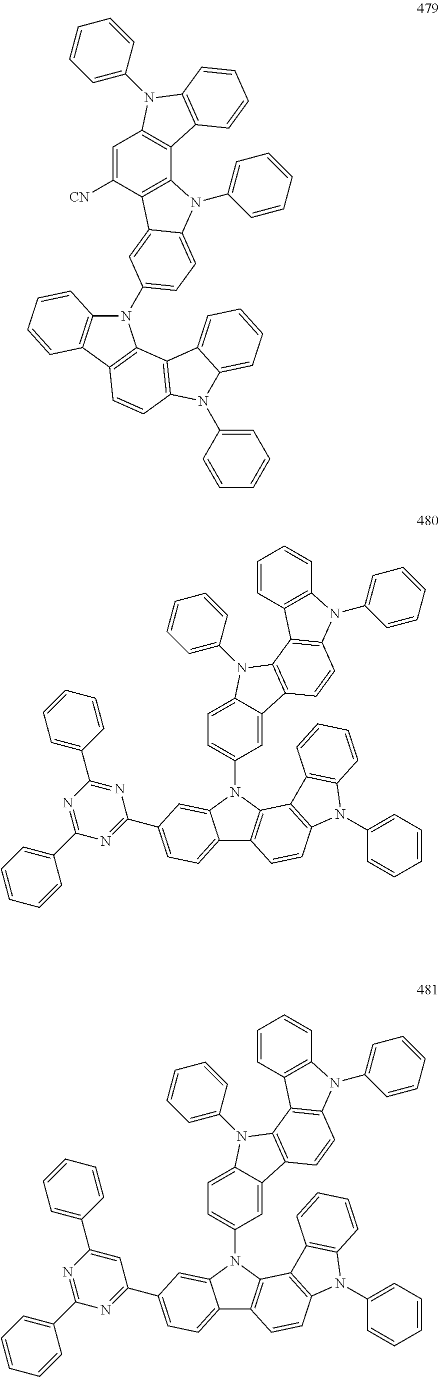

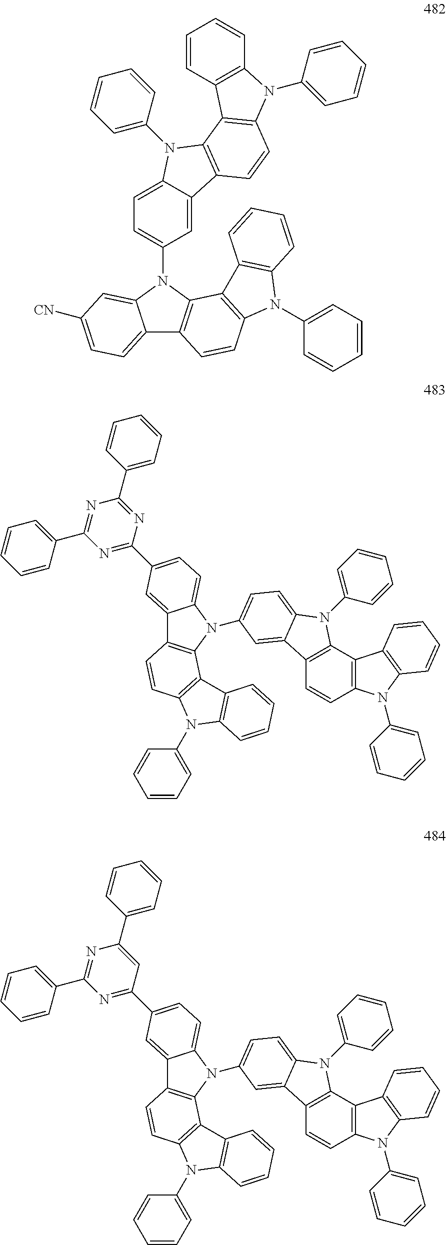

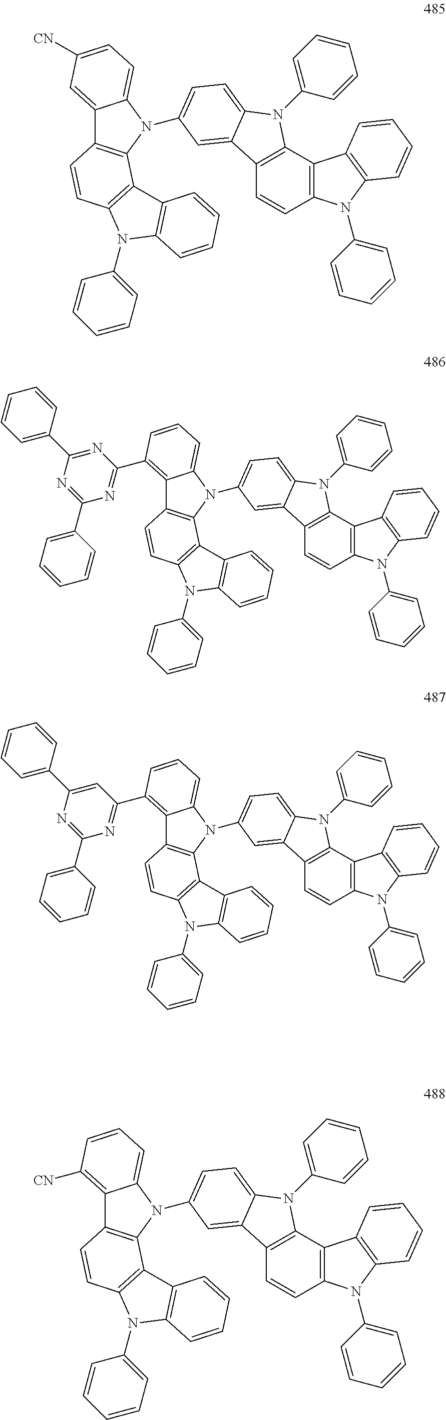

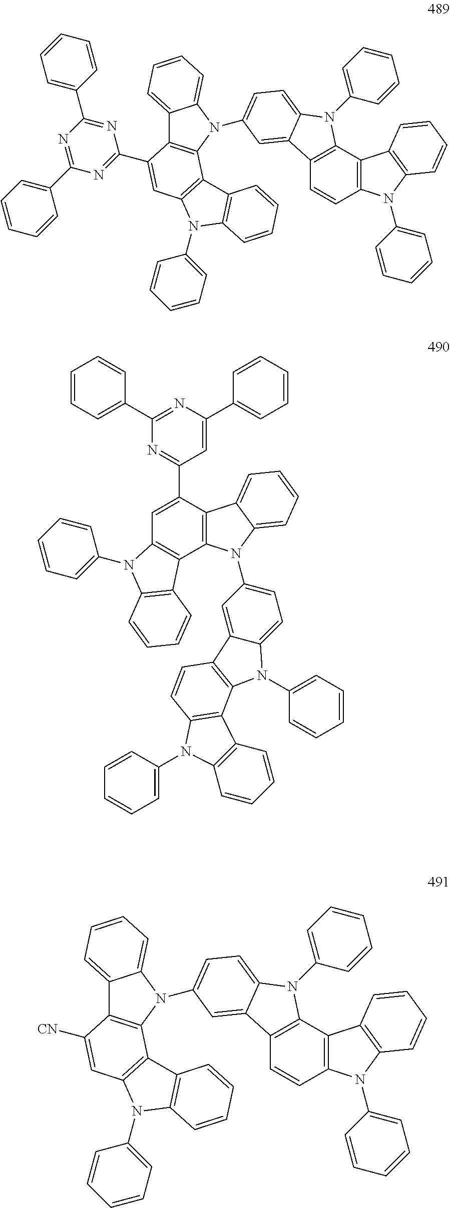

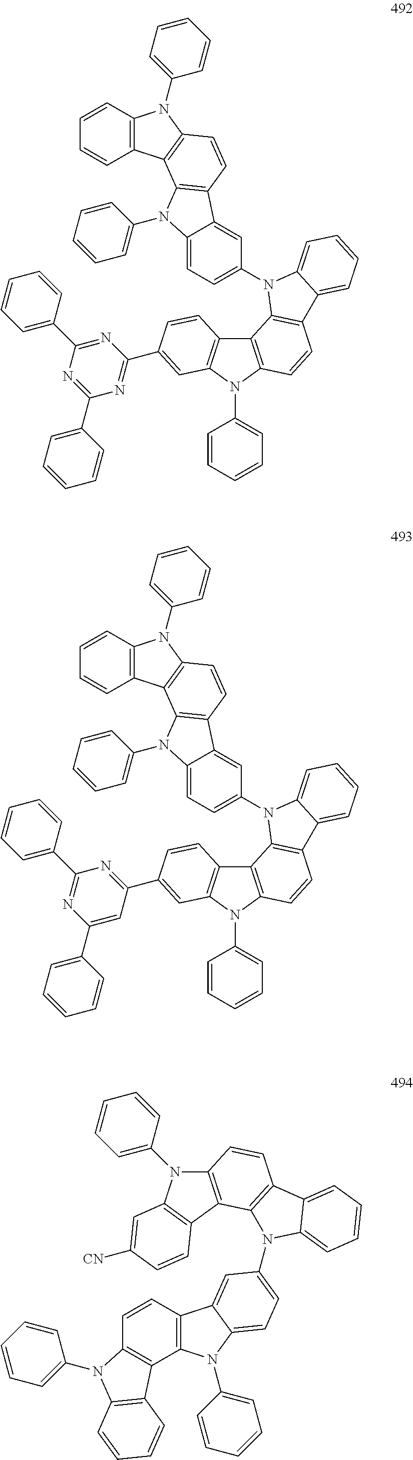

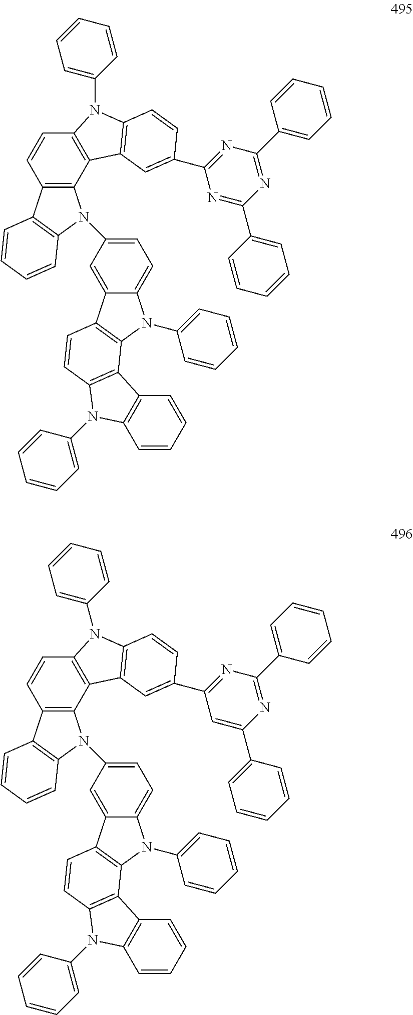 [SMILES: c1ccc(-c2nc(-c3ccccc3)nc(-c3ccc4c(c3)c3c(ccc5c6ccccc6n(-c6ccc7c(c6)c6ccc8c(c9ccccc9n8-c8ccccc8)c6n7-c6ccccc6)c53)n4-c3ccccc3)n2)cc1 ; c1ccc(-c2cc(-c3ccc4c(c3)c3c(ccc5c6ccccc6n(-c6ccc7c(c6)c6ccc8c(c9ccccc9n8-c8ccccc8)c6n7-c6ccccc6)c53)n4-c3ccccc3)nc(-c3ccccc3)n2)cc1]

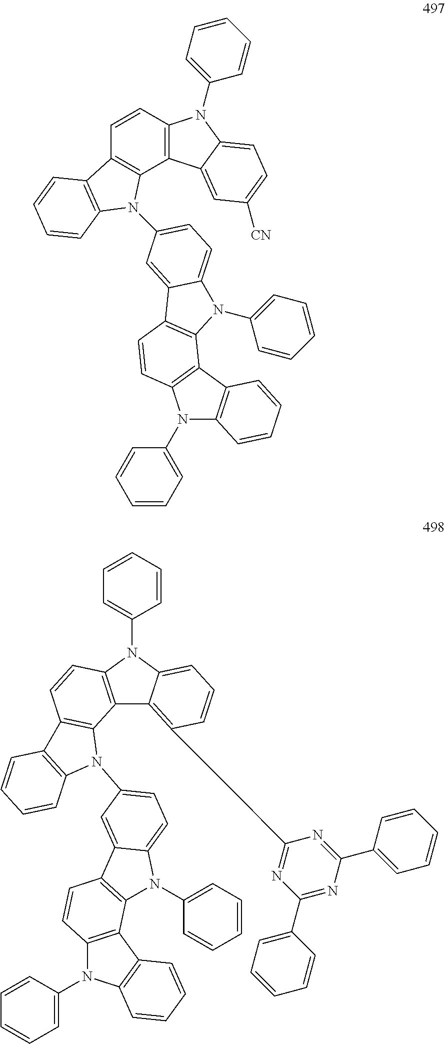 [SMILES: N#Cc1ccc2c(c1)c1c(ccc3c4ccccc4n(-c4ccc5c(c4)c4ccc6c(c7ccccc7n6-c6ccccc6)c4n5-c4ccccc4)c31)n2-c1ccccc1 ; c1ccc(-c2nc(-c3ccccc3)nc(-c3cccc4c3c3c(ccc5c6ccccc6n(-c6ccc7c(c6)c6ccc8c(c9ccccc9n8-c8ccccc8)c6n7-c6ccccc6)c53)n4-c3ccccc3)n2)cc1]

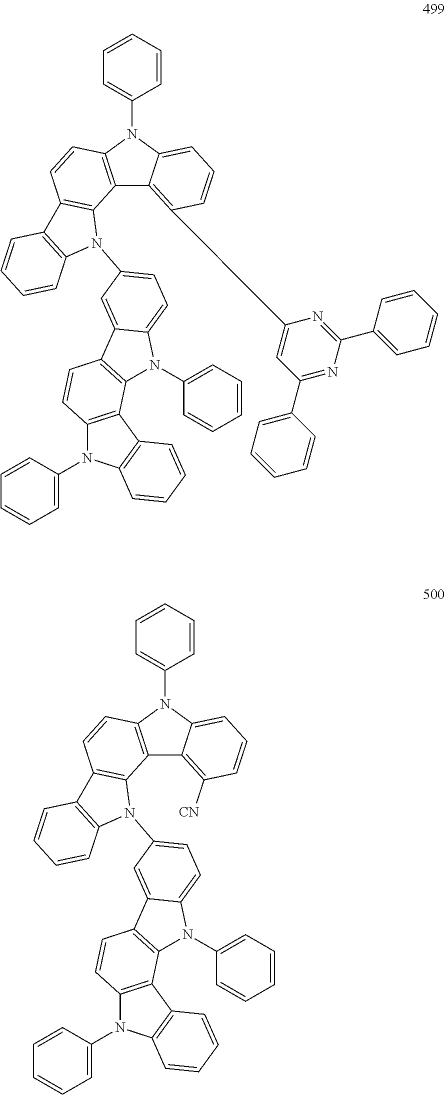

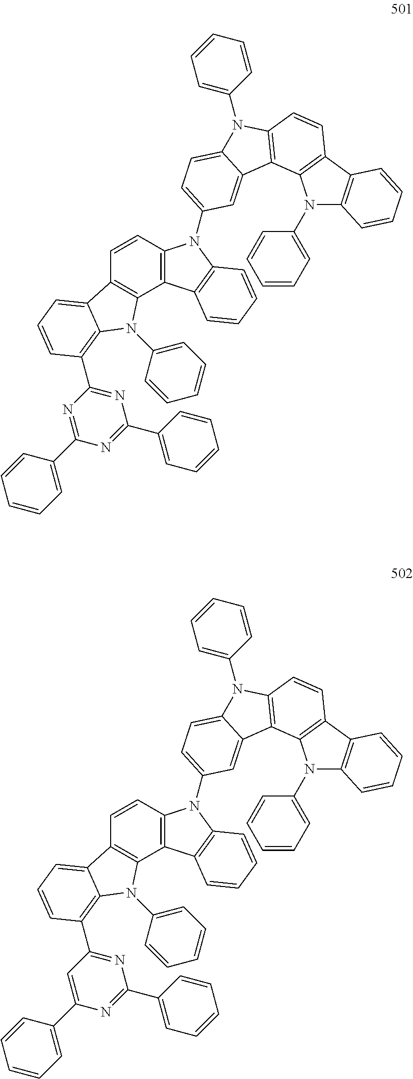 [SMILES: c1ccc(-c2nc(-c3ccccc3)nc(-c3cccc4c5ccc6c(c7ccccc7n6-c6ccc7c(c6)c6c(ccc8c9ccccc9n(-c9ccccc9)c86)n7-c6ccccc6)c5n(-c5ccccc5)c34)n2)cc1 ; c1ccc(-c2cc(-c3cccc4c5ccc6c(c7ccccc7n6-c6ccc7c(c6)c6c(ccc8c9ccccc9n(-c9ccccc9)c86)n7-c6ccccc6)c5n(-c5ccccc5)c34)nc(-c3ccccc3)n2)cc1]

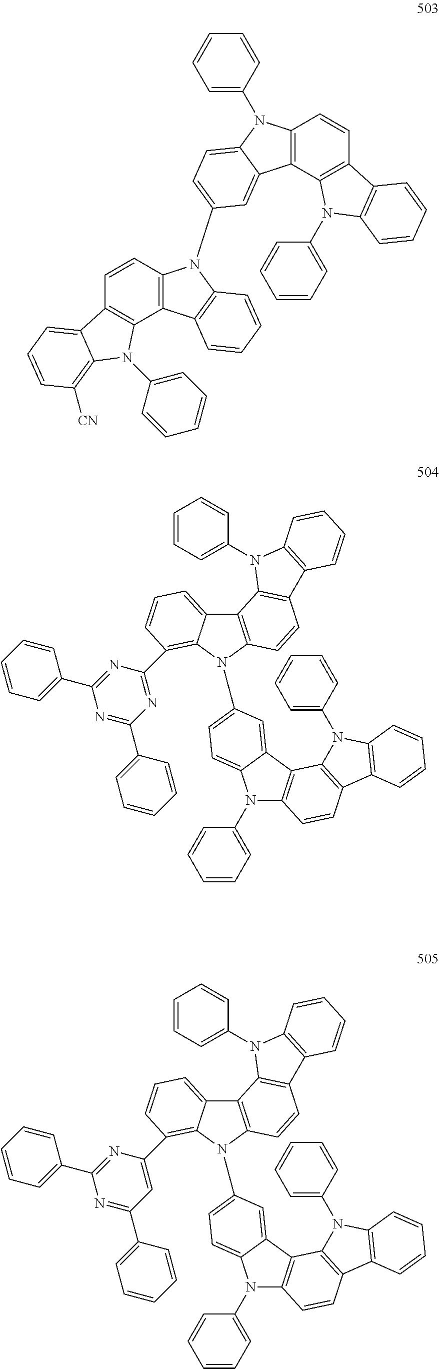 [SMILES: N#Cc1cccc2c3ccc4c(c5ccccc5n4-c4ccc5c(c4)c4c(ccc6c7ccccc7n(-c7ccccc7)c64)n5-c4ccccc4)c3n(-c3ccccc3)c12 ; c1ccc(-c2nc(-c3ccccc3)nc(-c3cccc4c5c(ccc6c7ccccc7n(-c7ccccc7)c65)n(-c5ccc6c(c5)c5c(ccc7c8ccccc8n(-c8ccccc8)c75)n6-c5ccccc5)c34)n2)cc1 ; c1ccc(-c2cc(-c3cccc4c5c(ccc6c7ccccc7n(-c7ccccc7)c65)n(-c5ccc6c(c5)c5c(ccc7c8ccccc8n(-c8ccccc8)c75)n6-c5ccccc5)c34)nc(-c3ccccc3)n2)cc1]

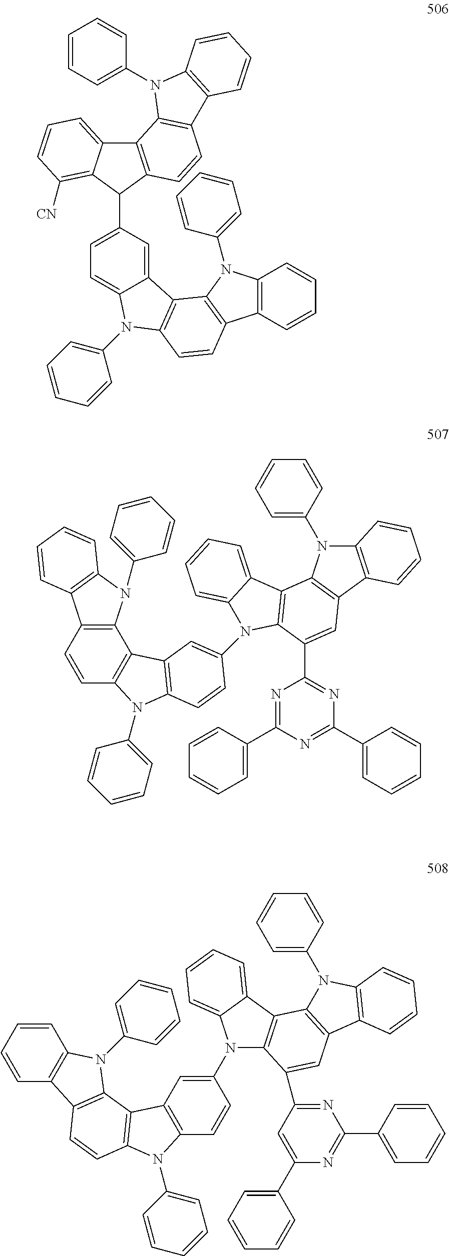

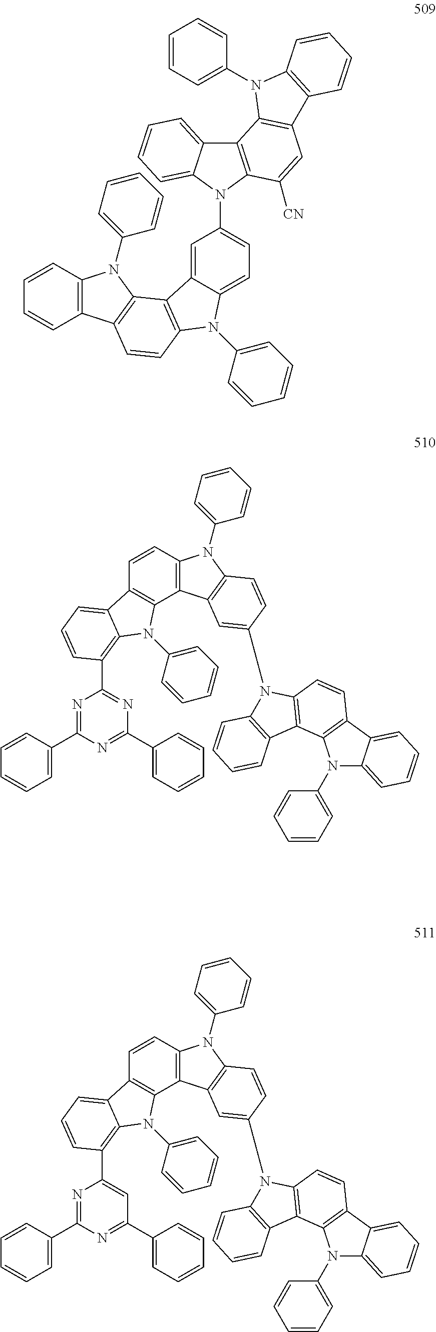

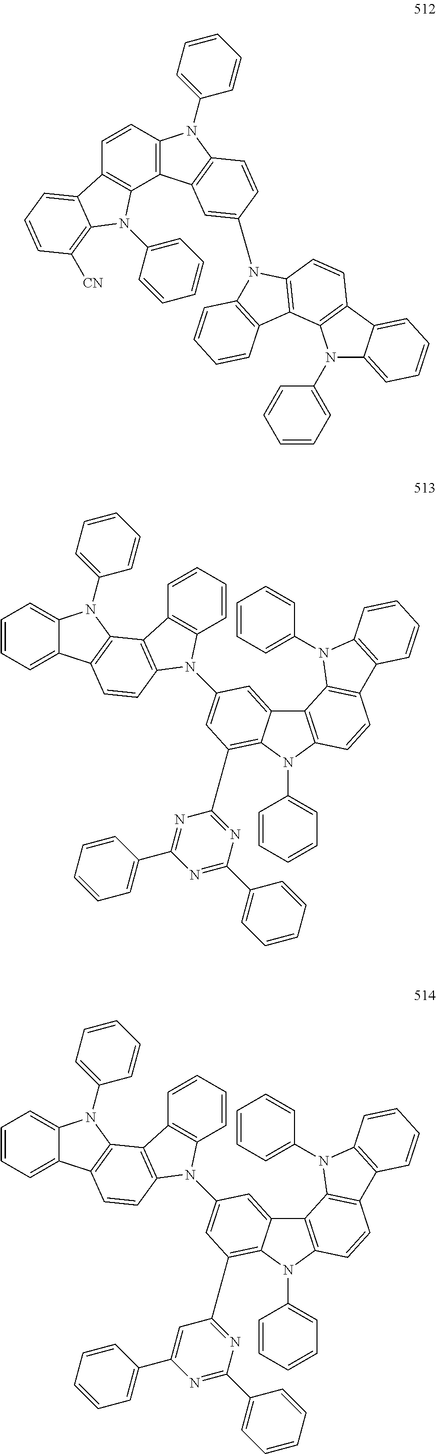 [SMILES: N#Cc1cccc2c3ccc4c(c5cc(-n6c7ccccc7c7c6ccc6c8ccccc8n(-c8ccccc8)c67)ccc5n4-c4ccccc4)c3n(-c3ccccc3)c12 ; c1ccc(-c2nc(-c3ccccc3)nc(-c3cc(-n4c5ccccc5c5c4ccc4c6ccccc6n(-c6ccccc6)c45)cc4c5c(ccc6c7ccccc7n(-c7ccccc7)c65)n(-c5ccccc5)c34)n2)cc1 ; c1ccc(-c2cc(-c3cc(-n4c5ccccc5c5c4ccc4c6ccccc6n(-c6ccccc6)c45)cc4c5c(ccc6c7ccccc7n(-c7ccccc7)c65)n(-c5ccccc5)c34)nc(-c3ccccc3)n2)cc1]

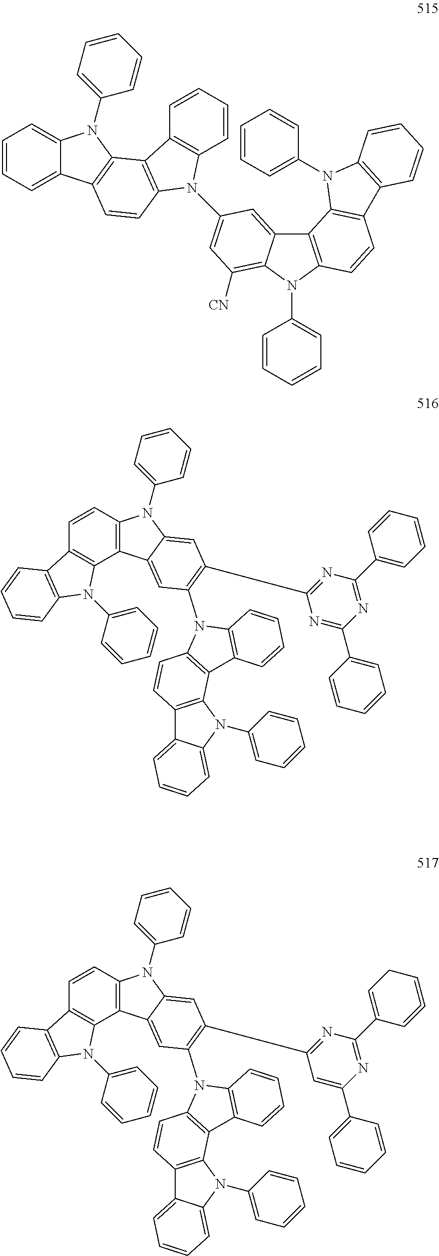

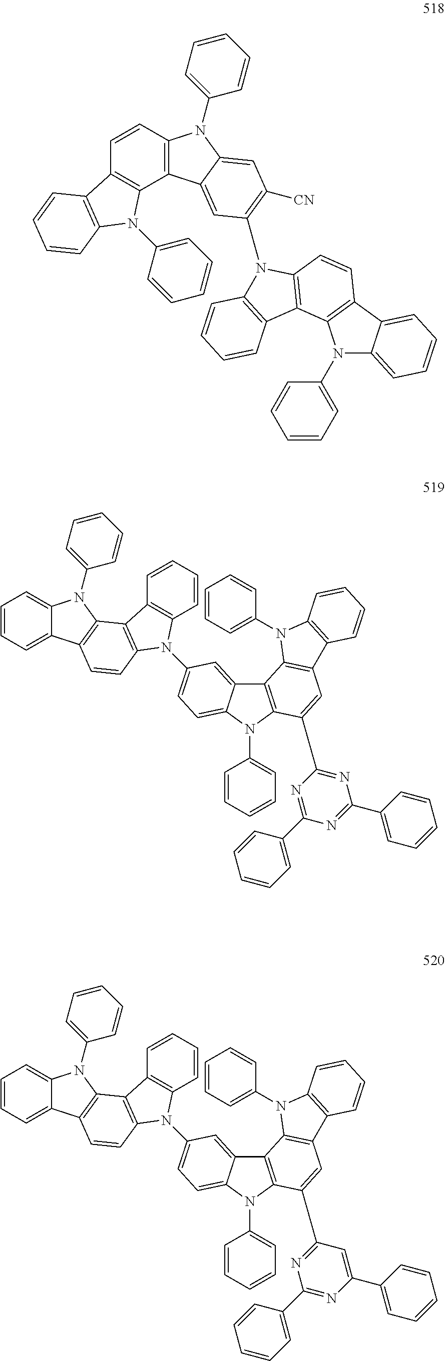 [SMILES: N#Cc1cc2c(cc1-n1c3ccccc3c3c1ccc1c4ccccc4n(-c4ccccc4)c13)c1c(ccc3c4ccccc4n(-c4ccccc4)c31)n2-c1ccccc1 ; c1ccc(-c2nc(-c3ccccc3)nc(-c3cc4c5ccccc5n(-c5ccccc5)c4c4c5cc(-n6c7ccccc7c7c6ccc6c8ccccc8n(-c8ccccc8)c67)ccc5n(-c5ccccc5)c34)n2)cc1 ; c1ccc(-c2cc(-c3cc4c5ccccc5n(-c5ccccc5)c4c4c5cc(-n6c7ccccc7c7c6ccc6c8ccccc8n(-c8ccccc8)c67)ccc5n(-c5ccccc5)c34)nc(-c3ccccc3)n2)cc1]

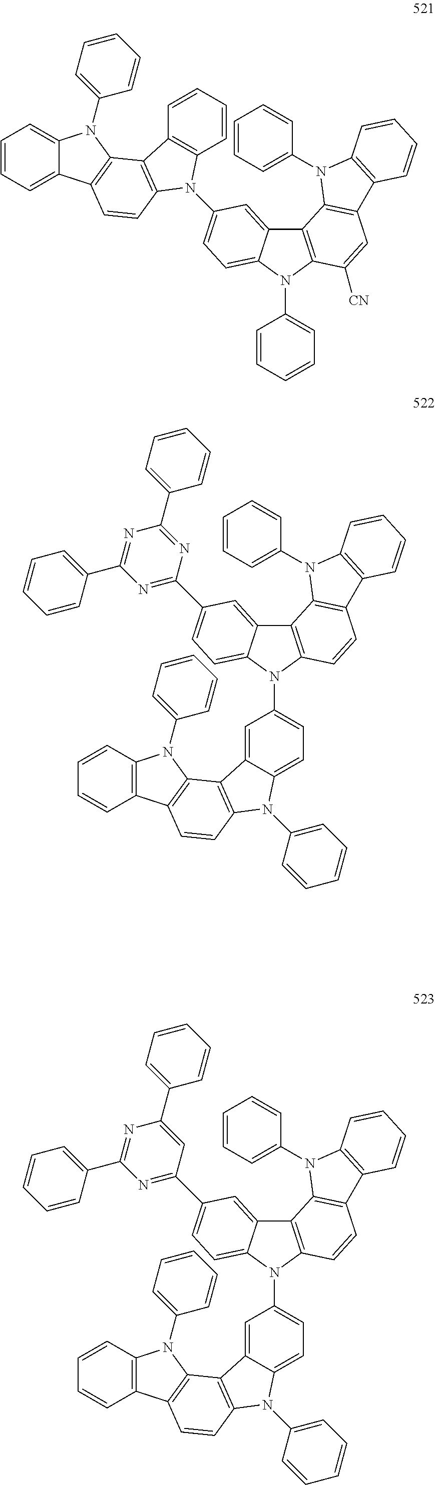 [SMILES: N#Cc1cc2c3ccccc3n(-c3ccccc3)c2c2c3cc(-n4c5ccccc5c5c4ccc4c6ccccc6n(-c6ccccc6)c45)ccc3n(-c3ccccc3)c12 ; c1ccc(-c2nc(-c3ccccc3)nc(-c3ccc4c(c3)c3c(ccc5c6ccccc6n(-c6ccccc6)c53)n4-c3ccc4c(c3)c3c(ccc5c6ccccc6n(-c6ccccc6)c53)n4-c3ccccc3)n2)cc1 ; c1ccc(-c2cc(-c3ccc4c(c3)c3c(ccc5c6ccccc6n(-c6ccccc6)c53)n4-c3ccc4c(c3)c3c(ccc5c6ccccc6n(-c6ccccc6)c53)n4-c3ccccc3)nc(-c3ccccc3)n2)cc1]

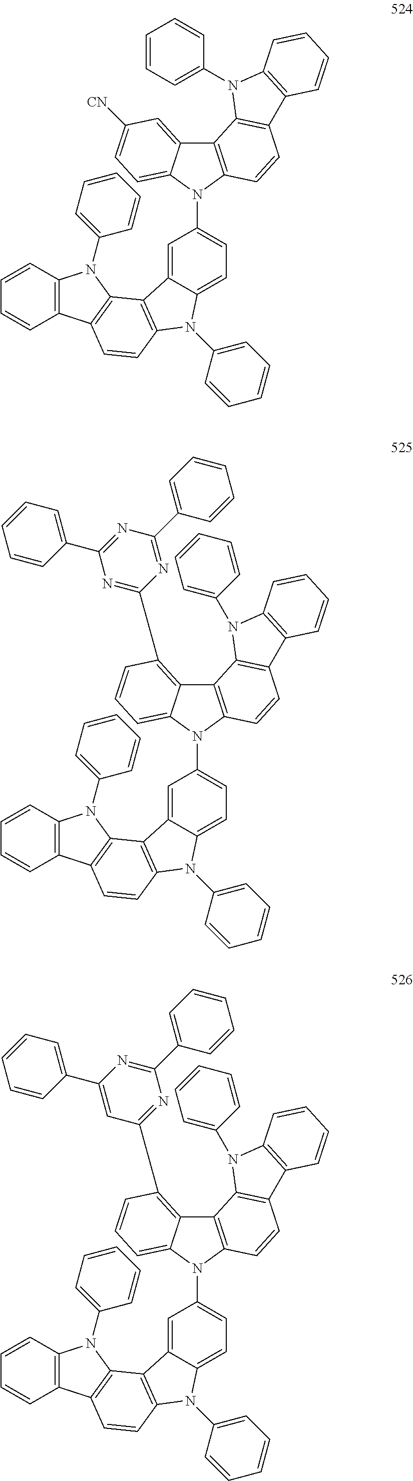

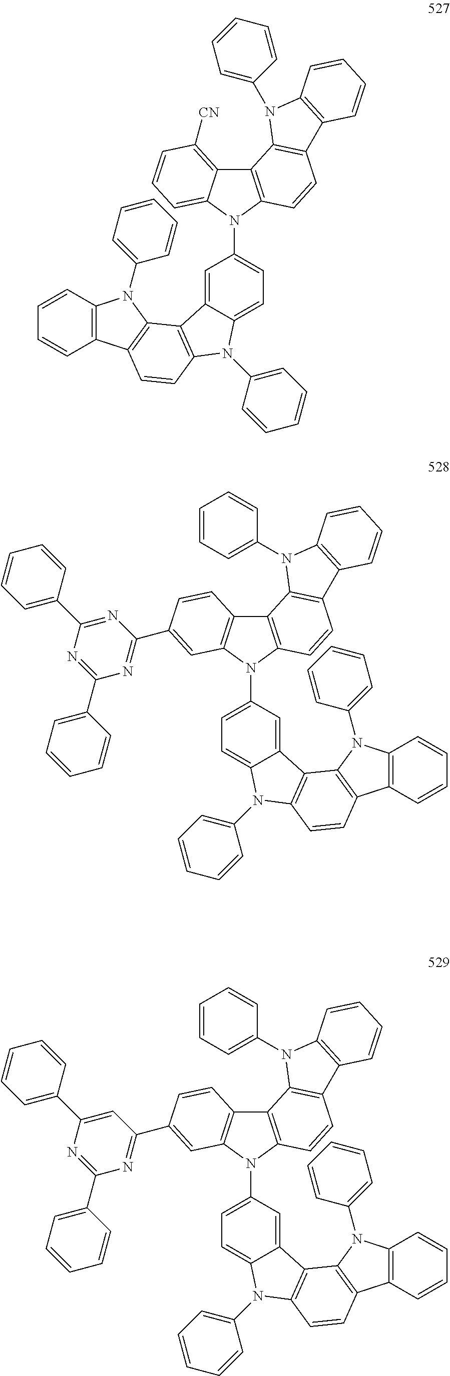 [SMILES: N#Cc1cccc2c1c1c(ccc3c4ccccc4n(-c4ccccc4)c31)n2-c1ccc2c(c1)c1c(ccc3c4ccccc4n(-c4ccccc4)c31)n2-c1ccccc1 ; c1ccc(-c2nc(-c3ccccc3)nc(-c3ccc4c5c(ccc6c7ccccc7n(-c7ccccc7)c65)n(-c5ccc6c(c5)c5c(ccc7c8ccccc8n(-c8ccccc8)c75)n6-c5ccccc5)c4c3)n2)cc1 ; c1ccc(-c2cc(-c3ccc4c5c(ccc6c7ccccc7n(-c7ccccc7)c65)n(-c5ccc6c(c5)c5c(ccc7c8ccccc8n(-c8ccccc8)c75)n6-c5ccccc5)c4c3)nc(-c3ccccc3)n2)cc1]

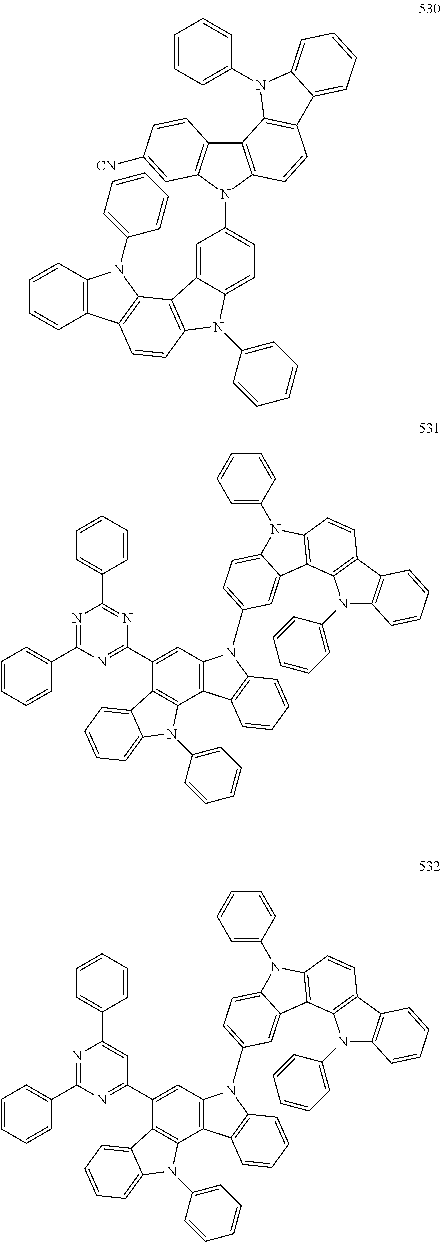

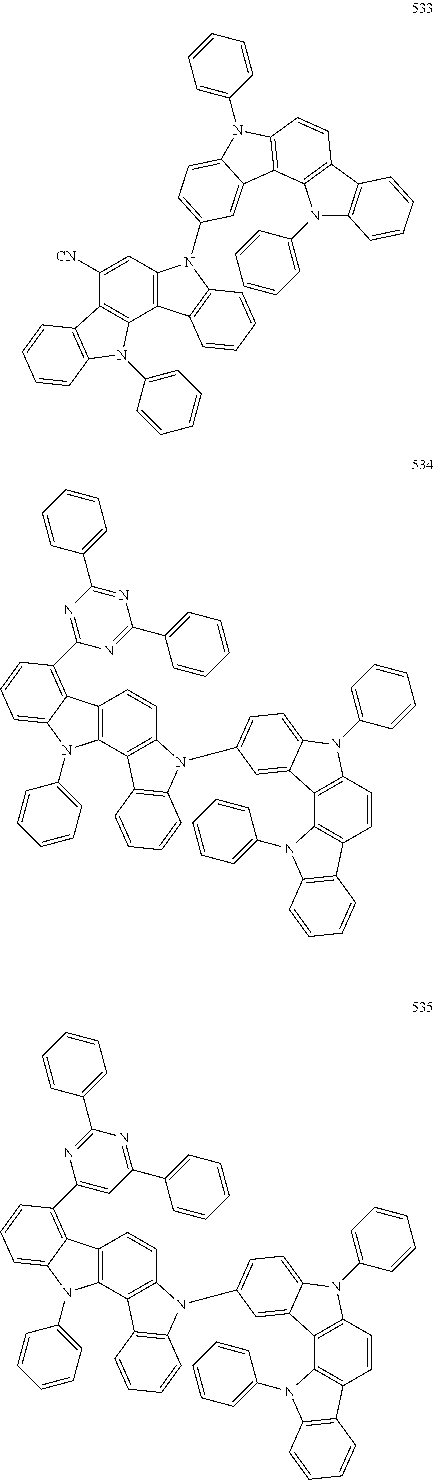

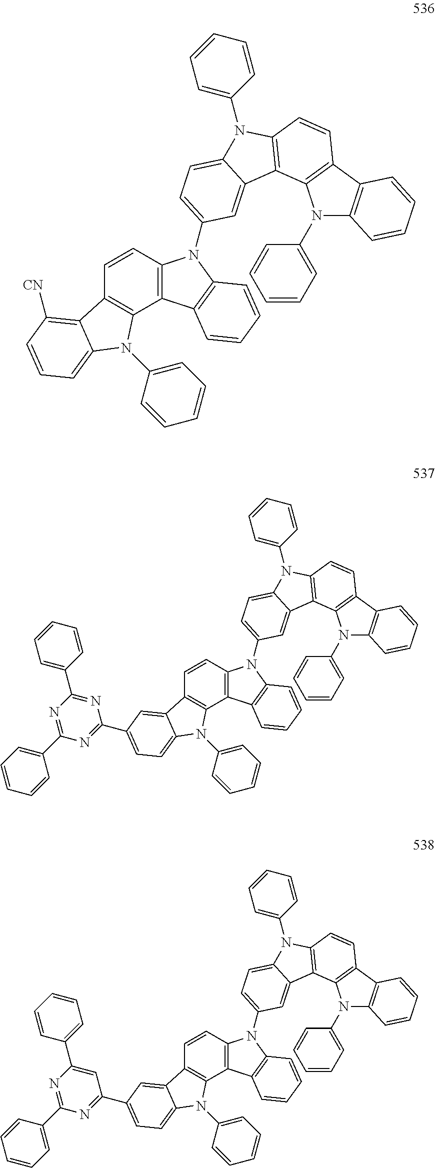

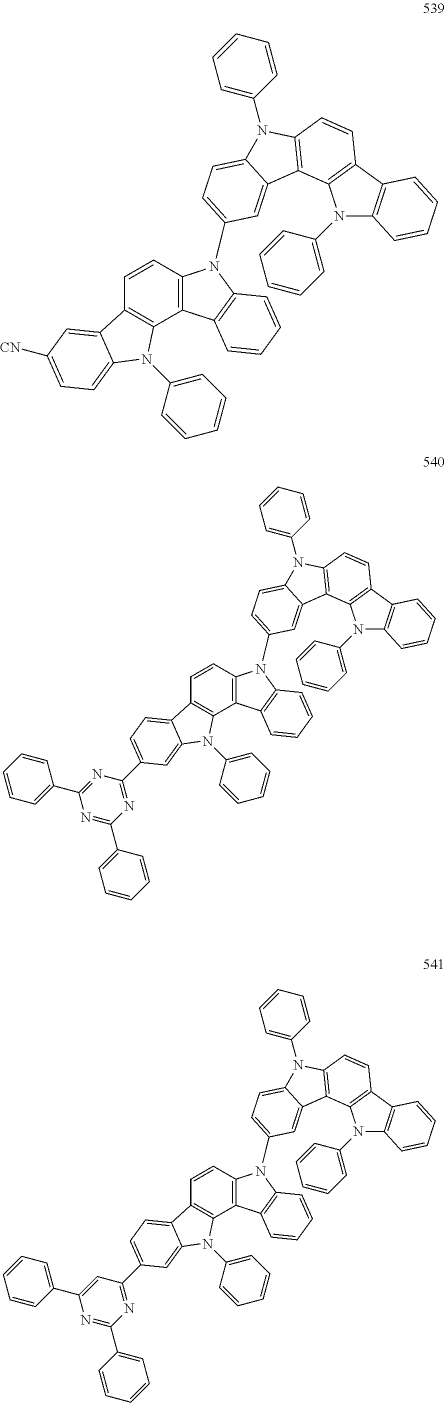

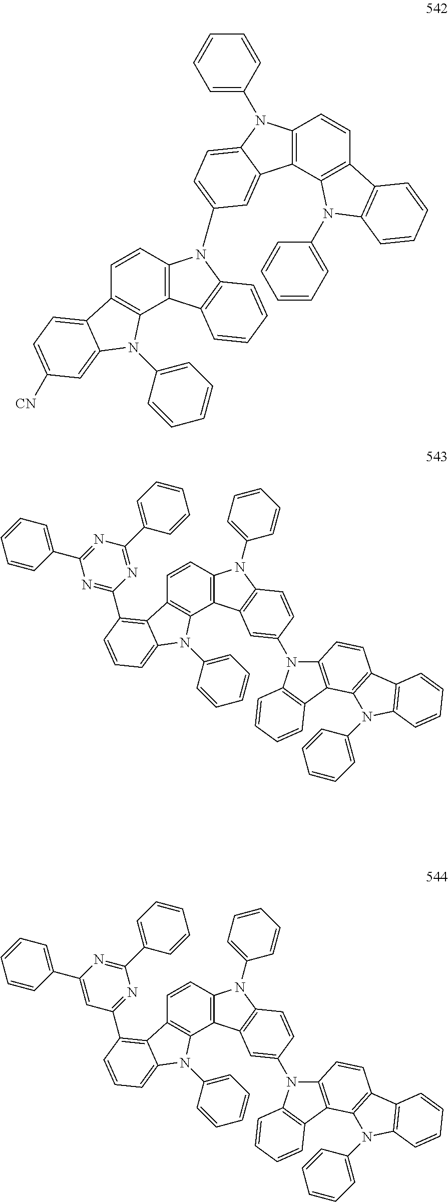

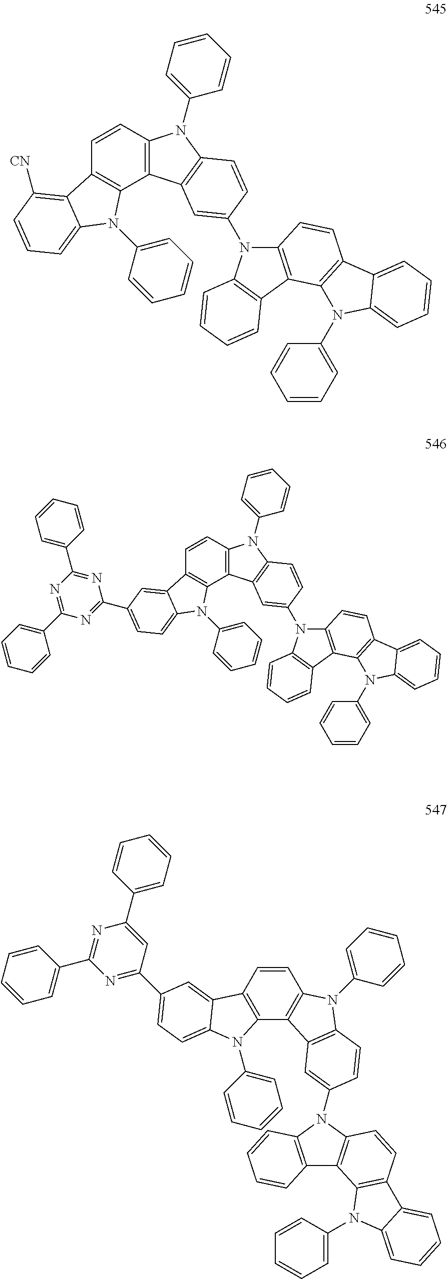

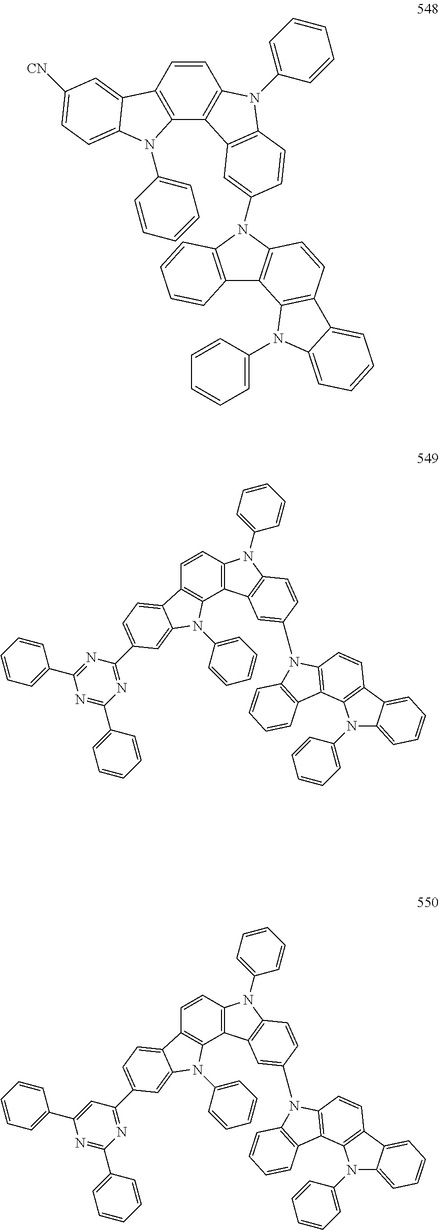

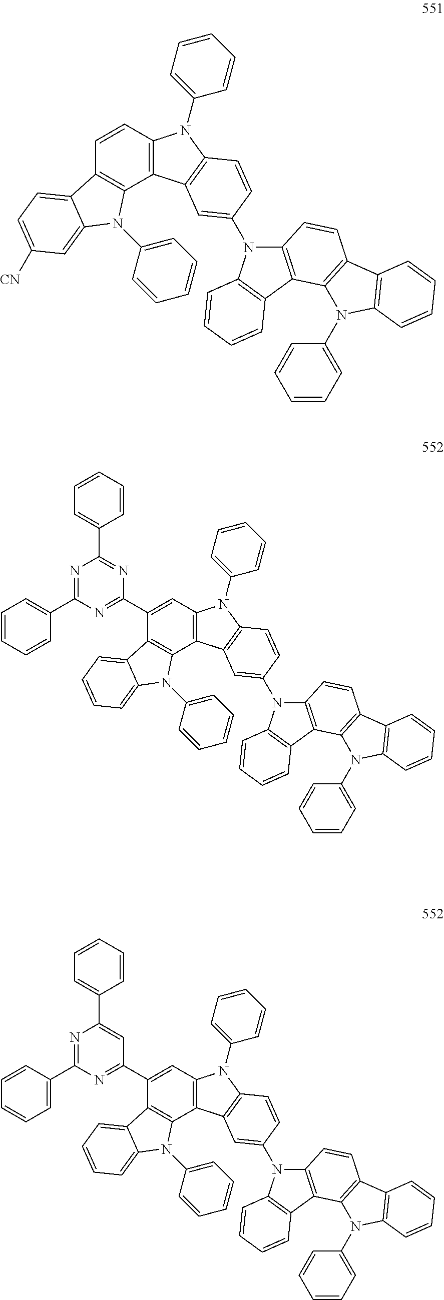

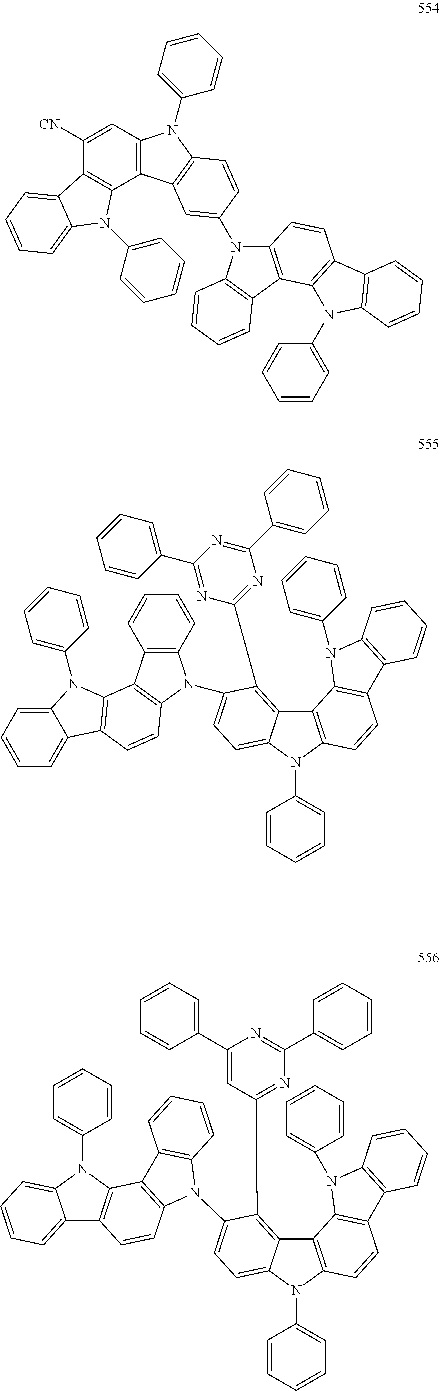

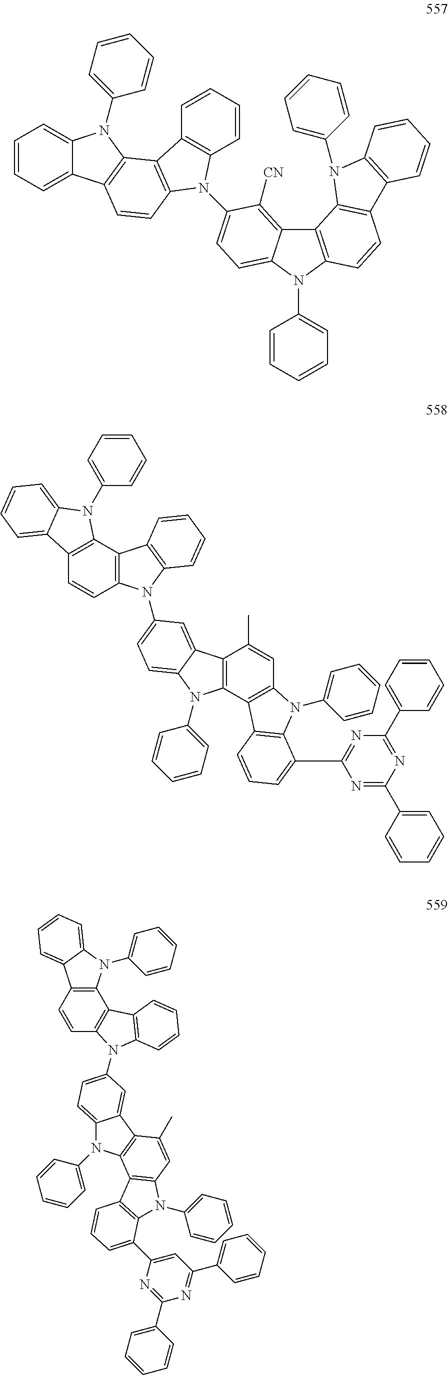 [SMILES: N#Cc1c(-n2c3ccccc3c3c2ccc2c4ccccc4n(-c4ccccc4)c23)ccc2c1c1c(ccc3c4ccccc4n(-c4ccccc4)c31)n2-c1ccccc1 ; Cc1cc2c(c3cccc(-c4nc(-c5ccccc5)nc(-c5ccccc5)n4)c3n2-c2ccccc2)c2c1c1cc(-n3c4ccccc4c4c3ccc3c5ccccc5n(-c5ccccc5)c34)ccc1n2-c1ccccc1 ; Cc1cc2c(c3cccc(-c4cc(-c5ccccc5)nc(-c5ccccc5)n4)c3n2-c2ccccc2)c2c1c1cc(-n3c4ccccc4c4c3ccc3c5ccccc5n(-c5ccccc5)c34)ccc1n2-c1ccccc1]

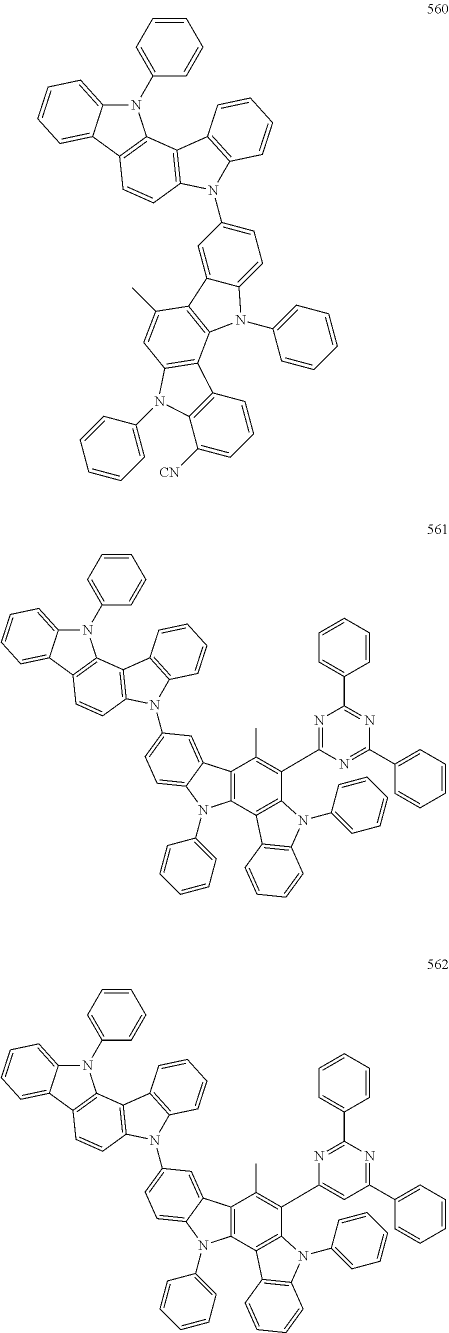

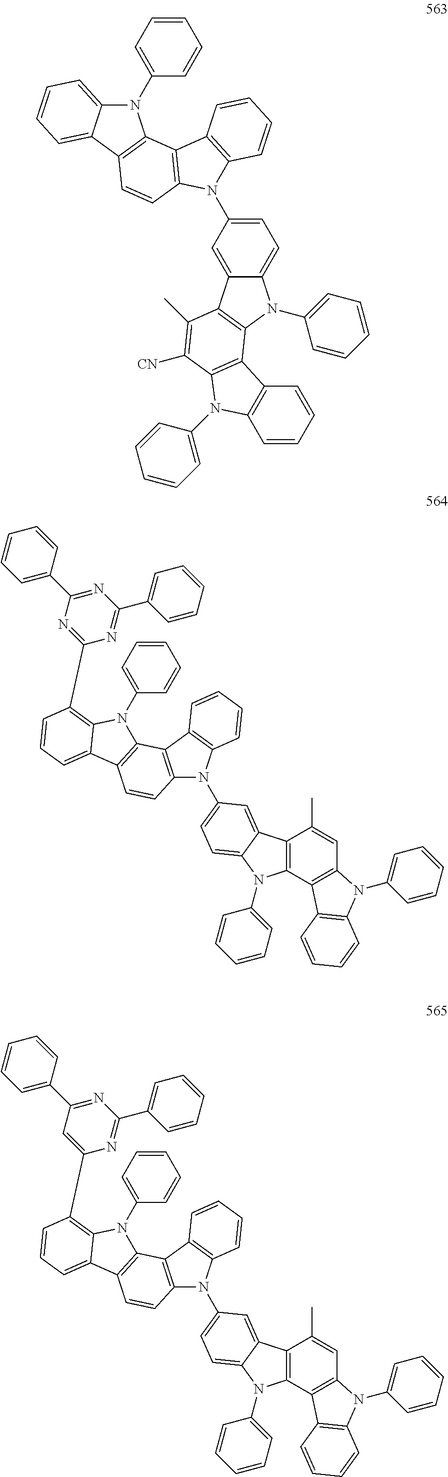

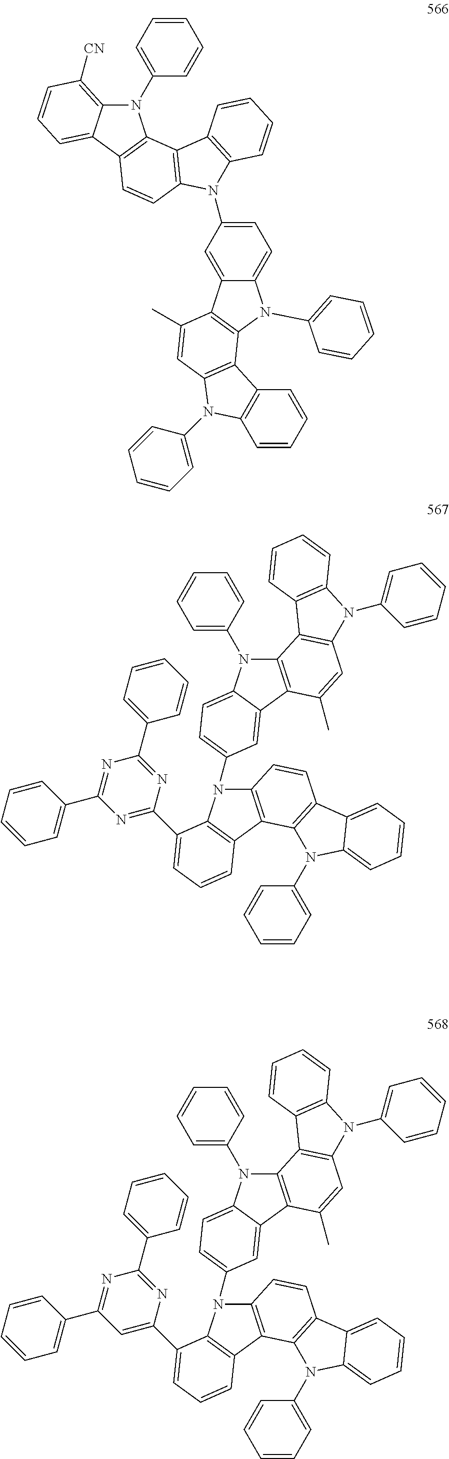 [SMILES: Cc1cc2c(c3ccccc3n2-c2ccccc2)c2c1c1cc(-n3c4ccccc4c4c3ccc3c5cccc(C#N)c5n(-c5ccccc5)c34)ccc1n2-c1ccccc1 ; Cc1cc2c(c3ccccc3n2-c2ccccc2)c2c1c1cc(-n3c4ccc5c6ccccc6n(-c6ccccc6)c5c4c4cccc(-c5nc(-c6ccccc6)nc(-c6ccccc6)n5)c43)ccc1n2-c1ccccc1 ; Cc1cc2c(c3ccccc3n2-c2ccccc2)c2c1c1cc(-n3c4ccc5c6ccccc6n(-c6ccccc6)c5c4c4cccc(-c5cc(-c6ccccc6)nc(-c6ccccc6)n5)c43)ccc1n2-c1ccccc1]

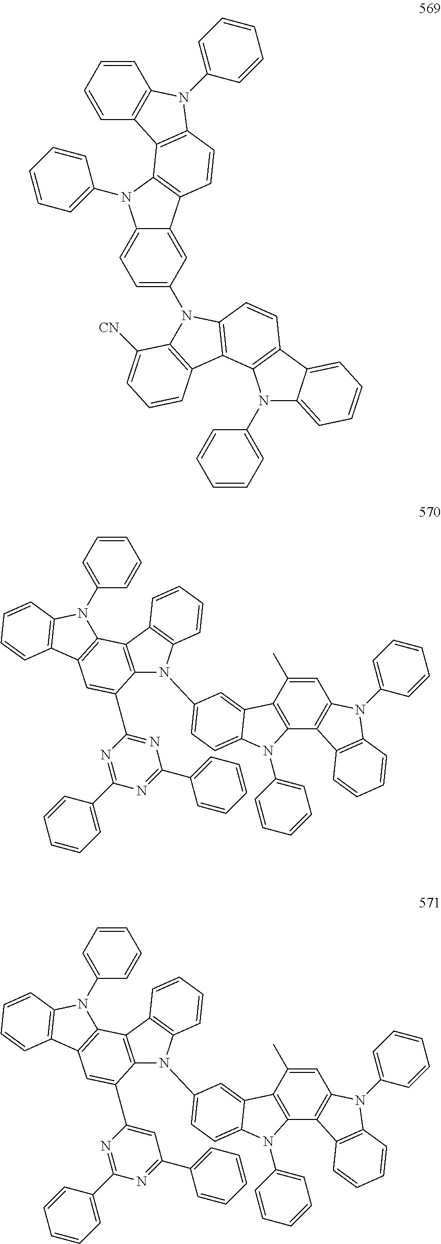

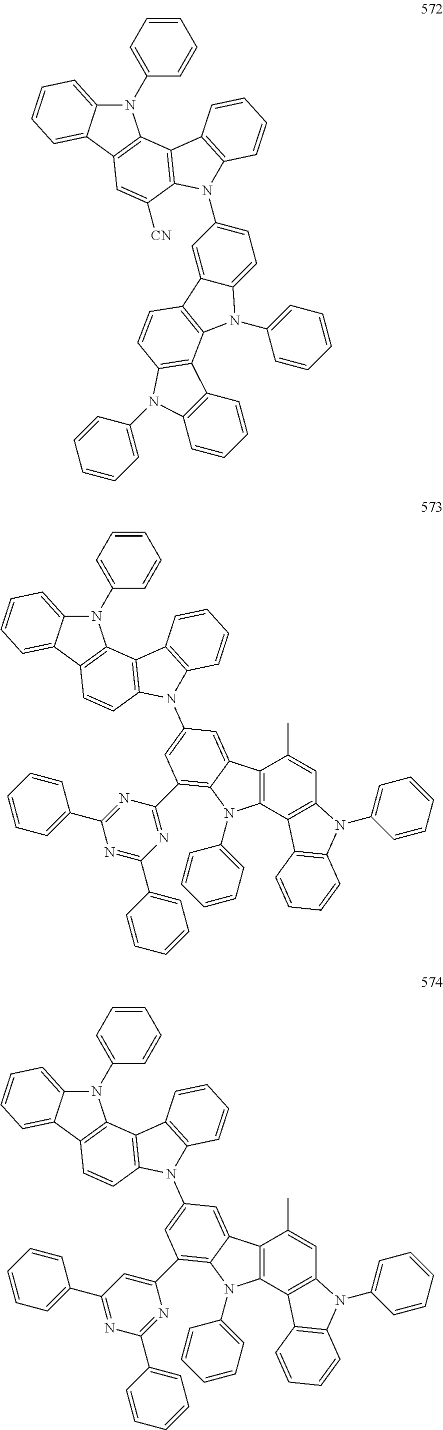 [SMILES: N#Cc1cc2c3ccccc3n(-c3ccccc3)c2c2c3ccccc3n(-c3ccc4c(c3)c3ccc5c(c6ccccc6n5-c5ccccc5)c3n4-c3ccccc3)c12 ; Cc1cc2c(c3ccccc3n2-c2ccccc2)c2c1c1cc(-n3c4ccccc4c4c3ccc3c5ccccc5n(-c5ccccc5)c34)cc(-c3nc(-c4ccccc4)nc(-c4ccccc4)n3)c1n2-c1ccccc1 ; Cc1cc2c(c3ccccc3n2-c2ccccc2)c2c1c1cc(-n3c4ccccc4c4c3ccc3c5ccccc5n(-c5ccccc5)c34)cc(-c3cc(-c4ccccc4)nc(-c4ccccc4)n3)c1n2-c1ccccc1]

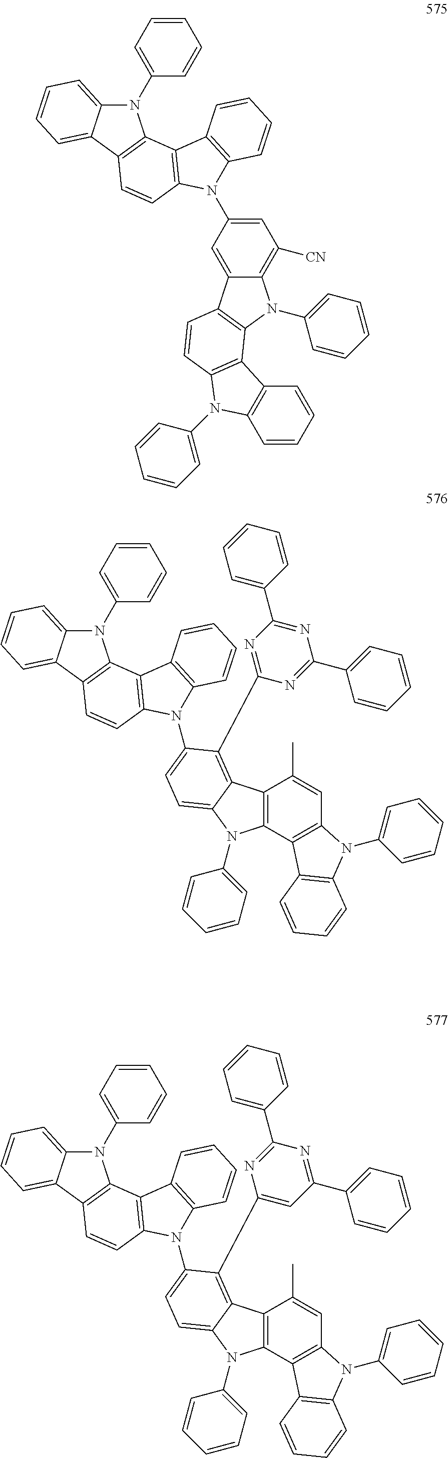 [SMILES: N#Cc1cc(-n2c3ccccc3c3c2ccc2c4ccccc4n(-c4ccccc4)c23)cc2c3ccc4c(c5ccccc5n4-c4ccccc4)c3n(-c3ccccc3)c12 ; Cc1cc2c(c3ccccc3n2-c2ccccc2)c2c1c1c(-c3nc(-c4ccccc4)nc(-c4ccccc4)n3)c(-n3c4ccccc4c4c3ccc3c5ccccc5n(-c5ccccc5)c34)ccc1n2-c1ccccc1 ; Cc1cc2c(c3ccccc3n2-c2ccccc2)c2c1c1c(-c3cc(-c4ccccc4)nc(-c4ccccc4)n3)c(-n3c4ccccc4c4c3ccc3c5ccccc5n(-c5ccccc5)c34)ccc1n2-c1ccccc1]

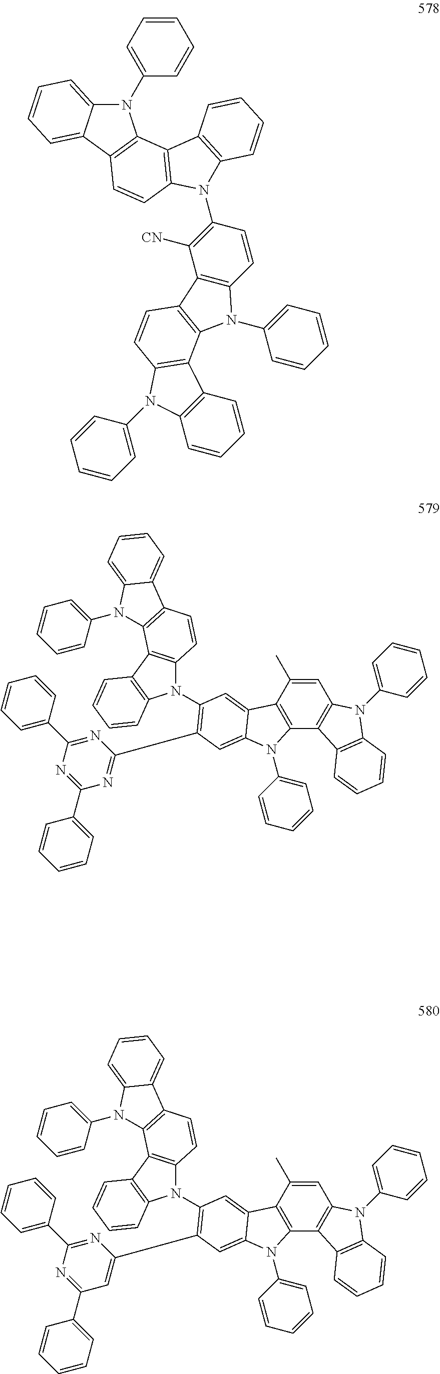

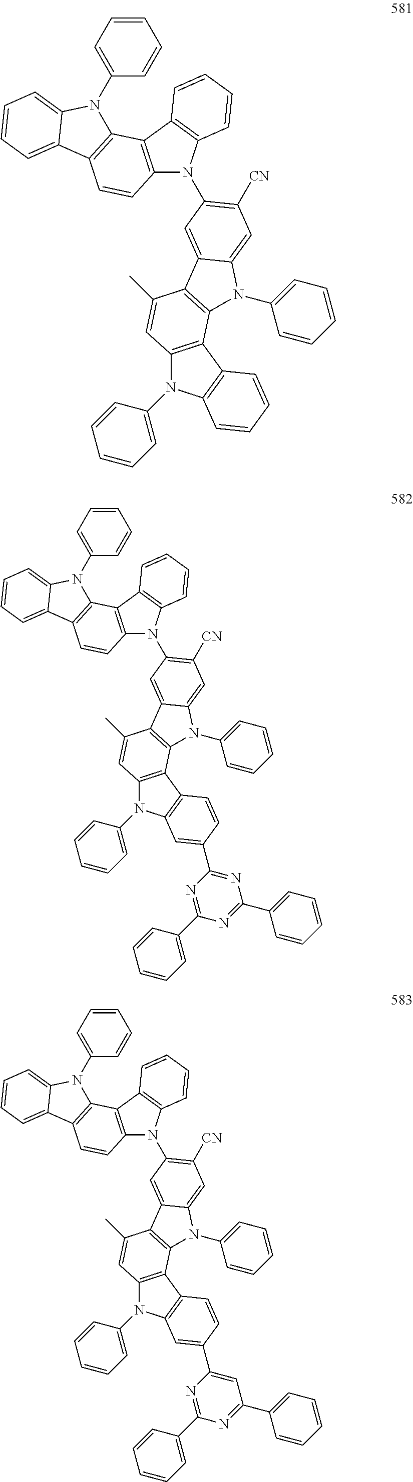 [SMILES: Cc1cc2c(c3ccccc3n2-c2ccccc2)c2c1c1cc(-n3c4ccccc4c4c3ccc3c5ccccc5n(-c5ccccc5)c34)c(C#N)cc1n2-c1ccccc1 ; Cc1cc2c(c3ccc(-c4nc(-c5ccccc5)nc(-c5ccccc5)n4)cc3n2-c2ccccc2)c2c1c1cc(-n3c4ccccc4c4c3ccc3c5ccccc5n(-c5ccccc5)c34)c(C#N)cc1n2-c1ccccc1 ; Cc1cc2c(c3ccc(-c4cc(-c5ccccc5)nc(-c5ccccc5)n4)cc3n2-c2ccccc2)c2c1c1cc(-n3c4ccccc4c4c3ccc3c5ccccc5n(-c5ccccc5)c34)c(C#N)cc1n2-c1ccccc1]

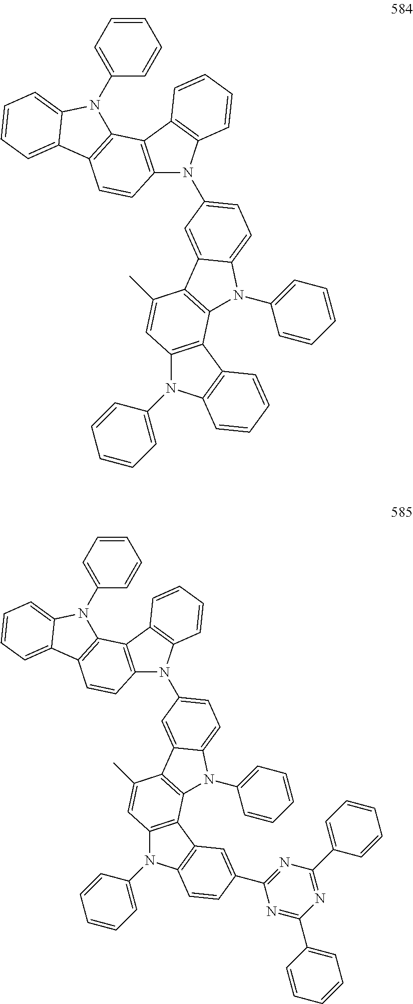 [SMILES: Cc1cc2c(c3ccccc3n2-c2ccccc2)c2c1c1cc(-n3c4ccccc4c4c3ccc3c5ccccc5n(-c5ccccc5)c34)ccc1n2-c1ccccc1 ; Cc1cc2c(c3cc(-c4nc(-c5ccccc5)nc(-c5ccccc5)n4)ccc3n2-c2ccccc2)c2c1c1cc(-n3c4ccccc4c4c3ccc3c5ccccc5n(-c5ccccc5)c34)ccc1n2-c1ccccc1]

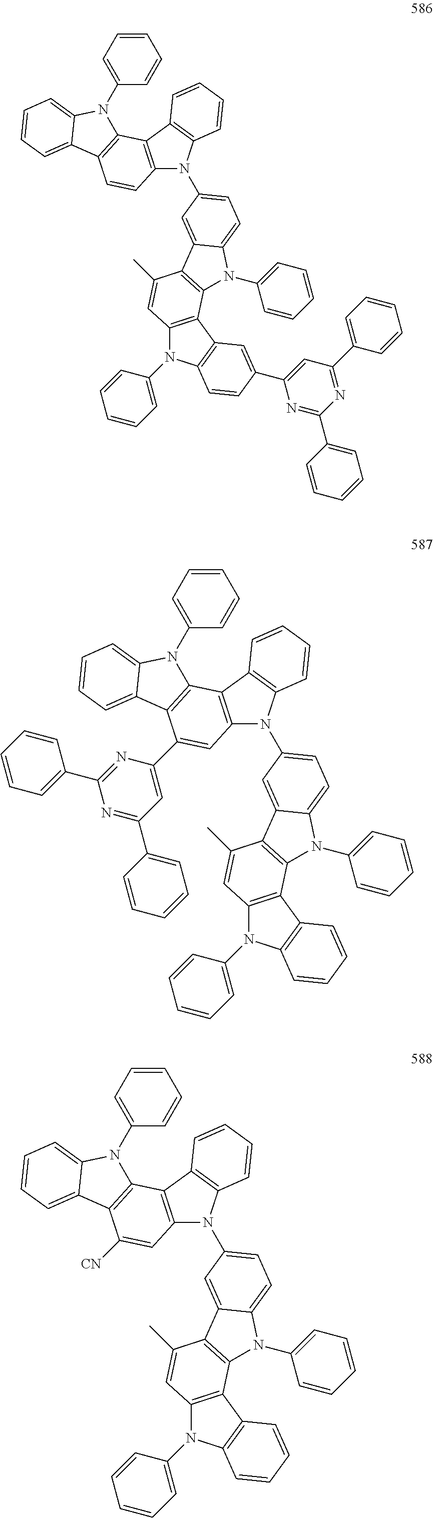

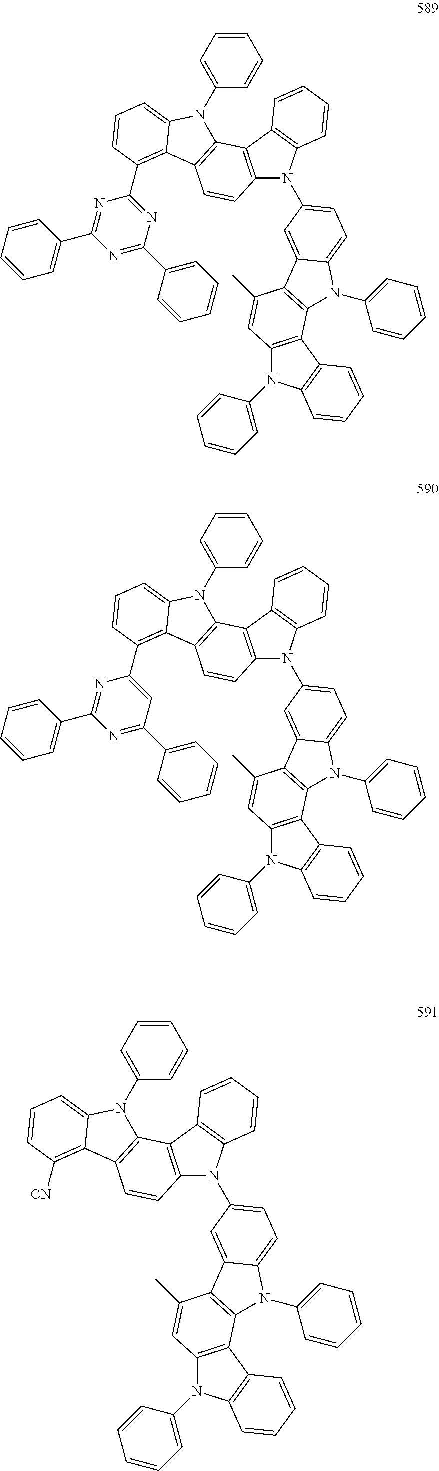

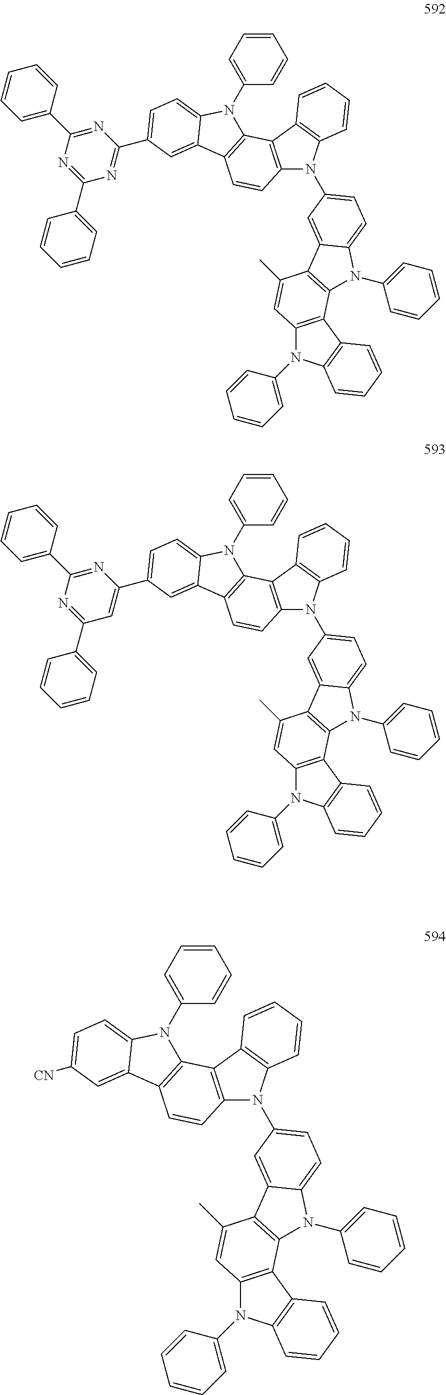 [SMILES: Cc1cc2c(c3ccccc3n2-c2ccccc2)c2c1c1cc(-n3c4ccccc4c4c3ccc3c5cc(-c6nc(-c7ccccc7)nc(-c7ccccc7)n6)ccc5n(-c5ccccc5)c34)ccc1n2-c1ccccc1 ; Cc1cc2c(c3ccccc3n2-c2ccccc2)c2c1c1cc(-n3c4ccccc4c4c3ccc3c5cc(-c6cc(-c7ccccc7)nc(-c7ccccc7)n6)ccc5n(-c5ccccc5)c34)ccc1n2-c1ccccc1 ; Cc1cc2c(c3ccccc3n2-c2ccccc2)c2c1c1cc(-n3c4ccccc4c4c3ccc3c5cc(C#N)ccc5n(-c5ccccc5)c34)ccc1n2-c1ccccc1]

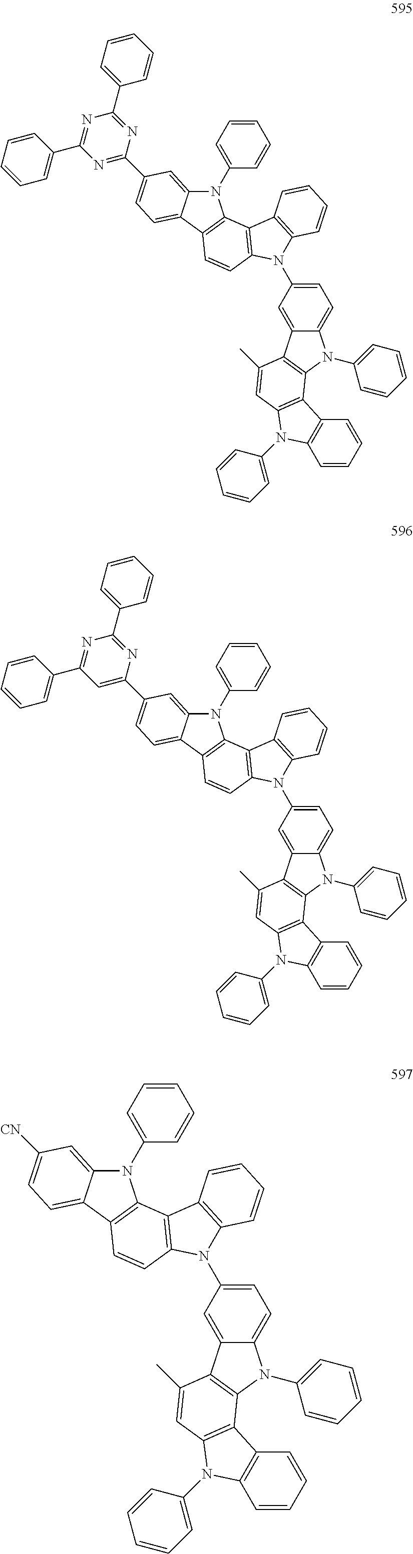

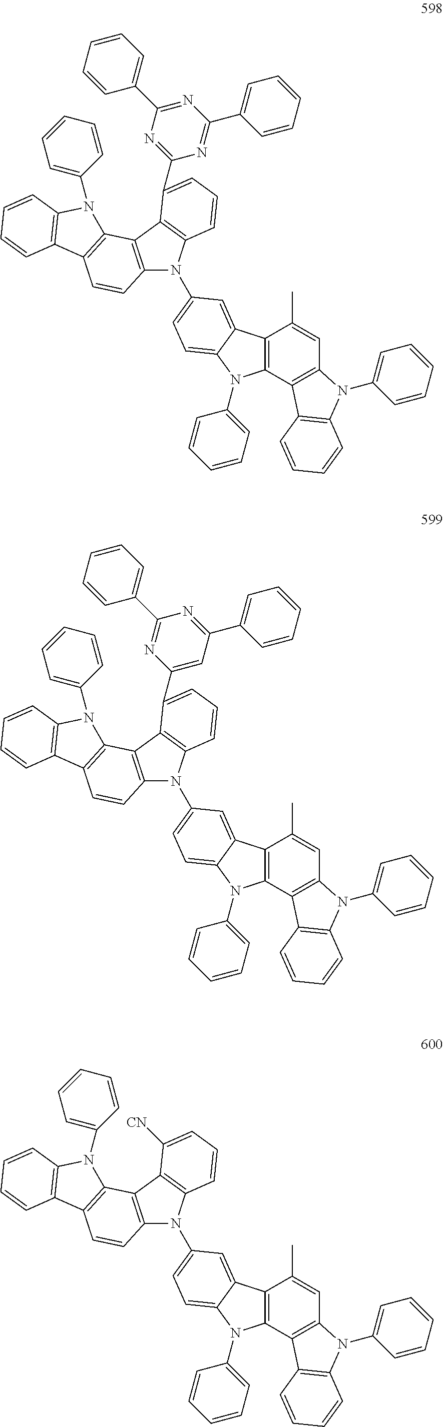

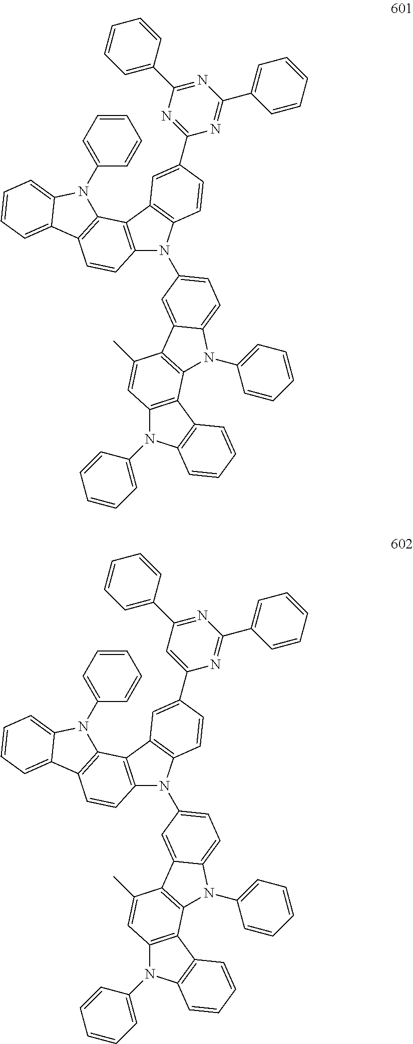 [SMILES: Cc1cc2c(c3ccccc3n2-c2ccccc2)c2c1c1cc(-n3c4ccc(-c5nc(-c6ccccc6)nc(-c6ccccc6)n5)cc4c4c3ccc3c5ccccc5n(-c5ccccc5)c34)ccc1n2-c1ccccc1 ; Cc1cc2c(c3ccccc3n2-c2ccccc2)c2c1c1cc(-n3c4ccc(-c5cc(-c6ccccc6)nc(-c6ccccc6)n5)cc4c4c3ccc3c5ccccc5n(-c5ccccc5)c34)ccc1n2-c1ccccc1]

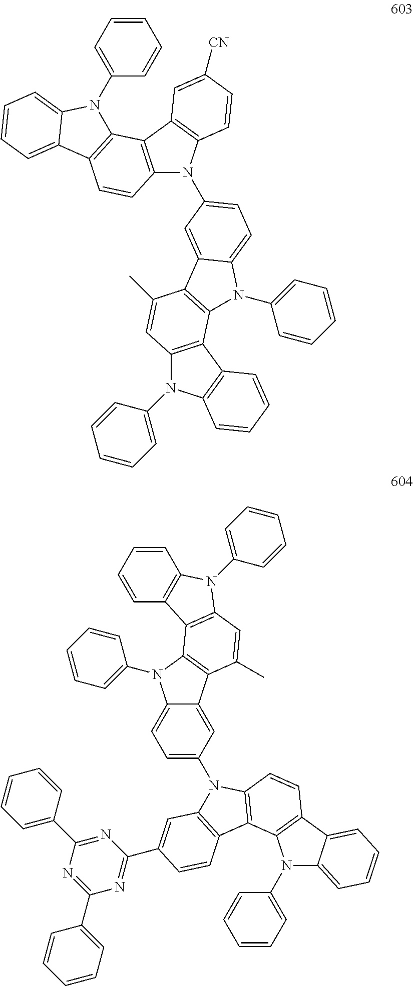 [SMILES: Cc1cc2c(c3ccccc3n2-c2ccccc2)c2c1c1cc(-n3c4ccc(C#N)cc4c4c3ccc3c5ccccc5n(-c5ccccc5)c34)ccc1n2-c1ccccc1 ; Cc1cc2c(c3ccccc3n2-c2ccccc2)c2c1c1cc(-n3c4cc(-c5nc(-c6ccccc6)nc(-c6ccccc6)n5)ccc4c4c3ccc3c5ccccc5n(-c5ccccc5)c34)ccc1n2-c1ccccc1]

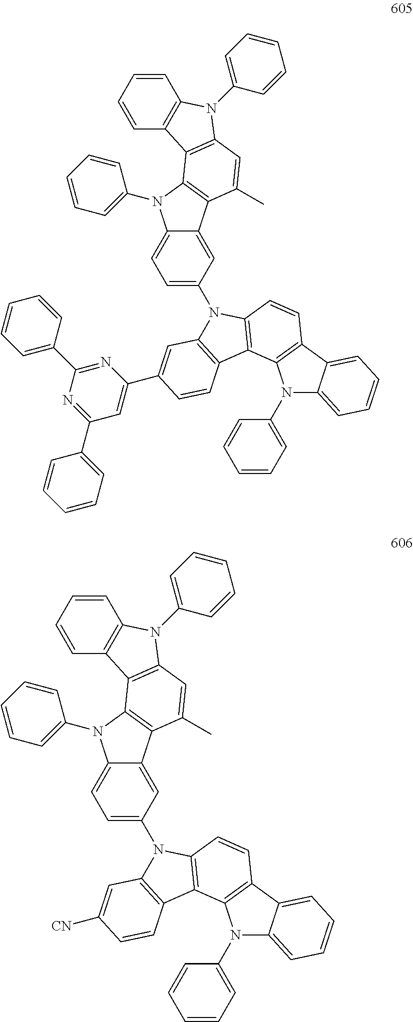

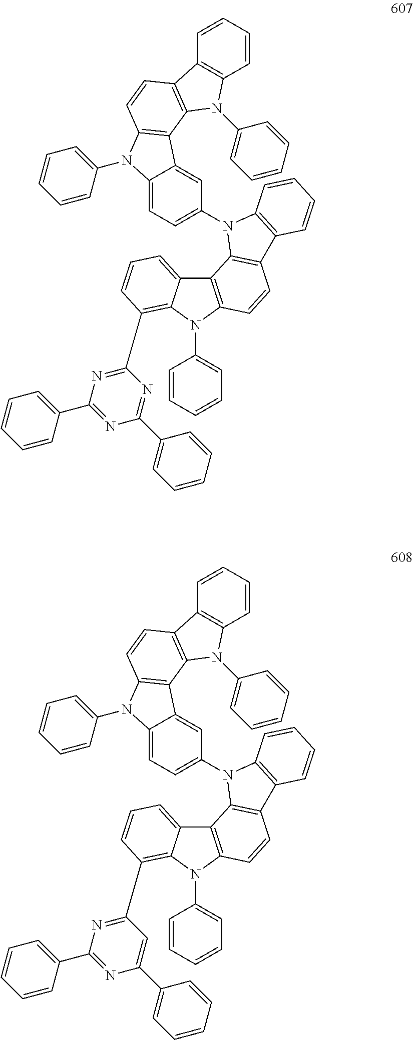 [SMILES: c1ccc(-c2nc(-c3ccccc3)nc(-c3cccc4c5c(ccc6c7ccccc7n(-c7ccc8c(c7)c7c(ccc9c%10ccccc%10n(-c%10ccccc%10)c97)n8-c7ccccc7)c65)n(-c5ccccc5)c34)n2)cc1 ; c1ccc(-c2cc(-c3cccc4c5c(ccc6c7ccccc7n(-c7ccc8c(c7)c7c(ccc9c%10ccccc%10n(-c%10ccccc%10)c97)n8-c7ccccc7)c65)n(-c5ccccc5)c34)nc(-c3ccccc3)n2)cc1]

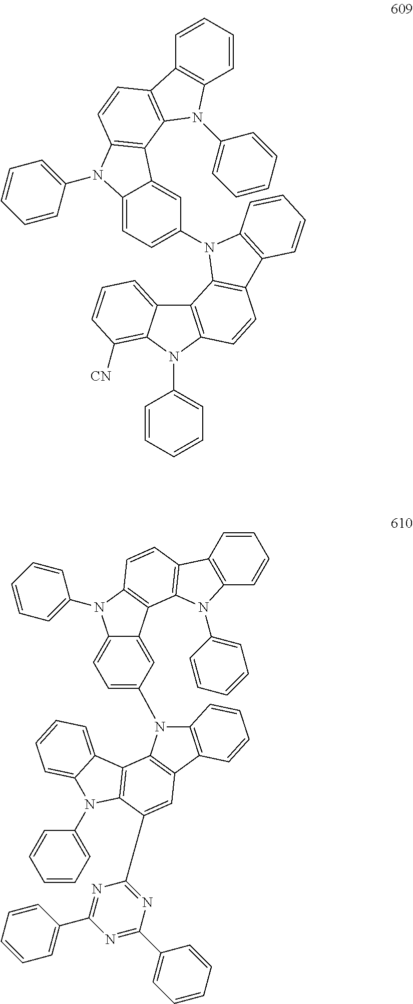

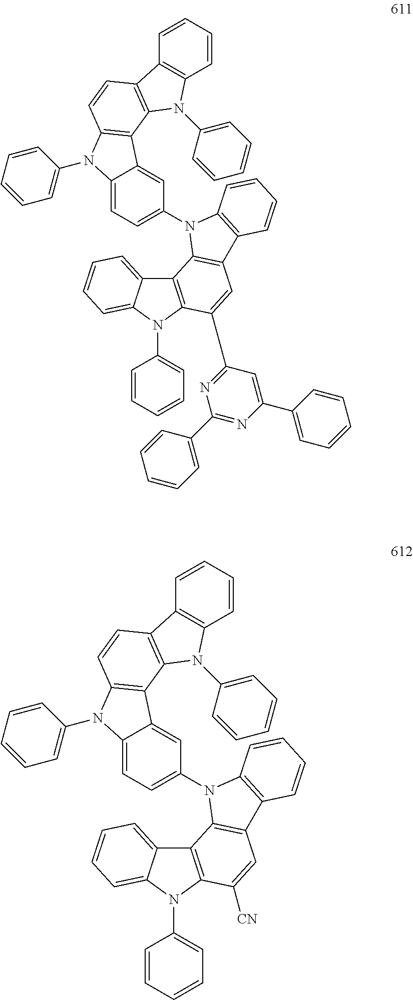 [SMILES: c1ccc(-c2cc(-c3cc4c5ccccc5n(-c5ccc6c(c5)c5c(ccc7c8ccccc8n(-c8ccccc8)c75)n6-c5ccccc5)c4c4c5ccccc5n(-c5ccccc5)c34)nc(-c3ccccc3)n2)cc1 ; N#Cc1cc2c3ccccc3n(-c3ccc4c(c3)c3c(ccc5c6ccccc6n(-c6ccccc6)c53)n4-c3ccccc3)c2c2c3ccccc3n(-c3ccccc3)c12]

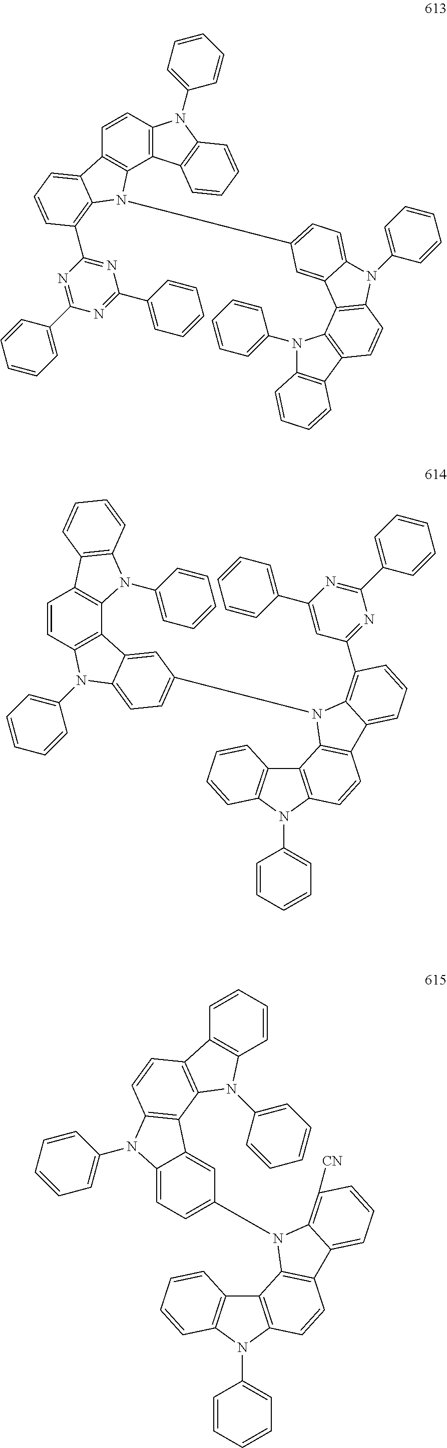 [SMILES: c1ccc(-c2nc(-c3ccccc3)nc(-c3cccc4c5ccc6c(c7ccccc7n6-c6ccccc6)c5n(-c5ccc6c(c5)c5c(ccc7c8ccccc8n(-c8ccccc8)c75)n6-c5ccccc5)c34)n2)cc1 ; c1ccc(-c2cc(-c3cccc4c5ccc6c(c7ccccc7n6-c6ccccc6)c5n(-c5ccc6c(c5)c5c(ccc7c8ccccc8n(-c8ccccc8)c75)n6-c5ccccc5)c34)nc(-c3ccccc3)n2)cc1 ; N#Cc1cccc2c3ccc4c(c5ccccc5n4-c4ccccc4)c3n(-c3ccc4c(c3)c3c(ccc5c6ccccc6n(-c6ccccc6)c53)n4-c3ccccc3)c12]

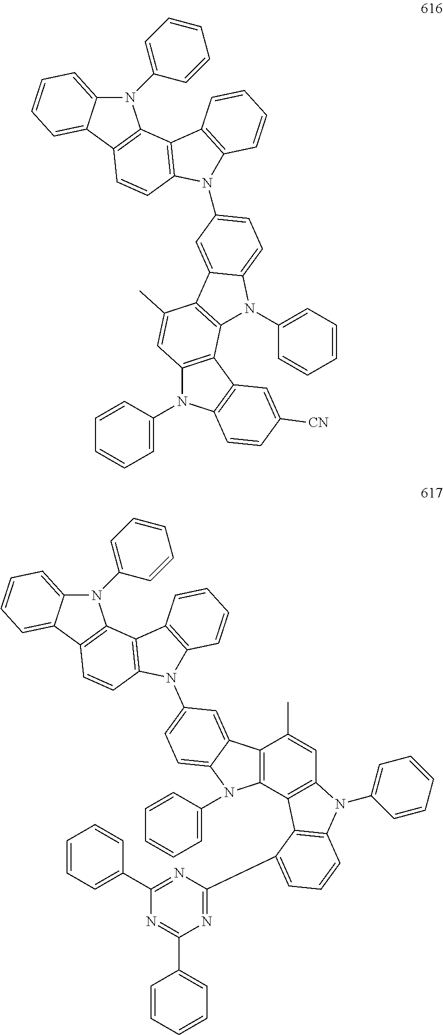 [SMILES: Cc1cc2c(c3cc(C#N)ccc3n2-c2ccccc2)c2c1c1cc(-n3c4ccccc4c4c3ccc3c5ccccc5n(-c5ccccc5)c34)ccc1n2-c1ccccc1 ; Cc1cc2c(c3c(-c4nc(-c5ccccc5)nc(-c5ccccc5)n4)cccc3n2-c2ccccc2)c2c1c1cc(-n3c4ccccc4c4c3ccc3c5ccccc5n(-c5ccccc5)c34)ccc1n2-c1ccccc1]

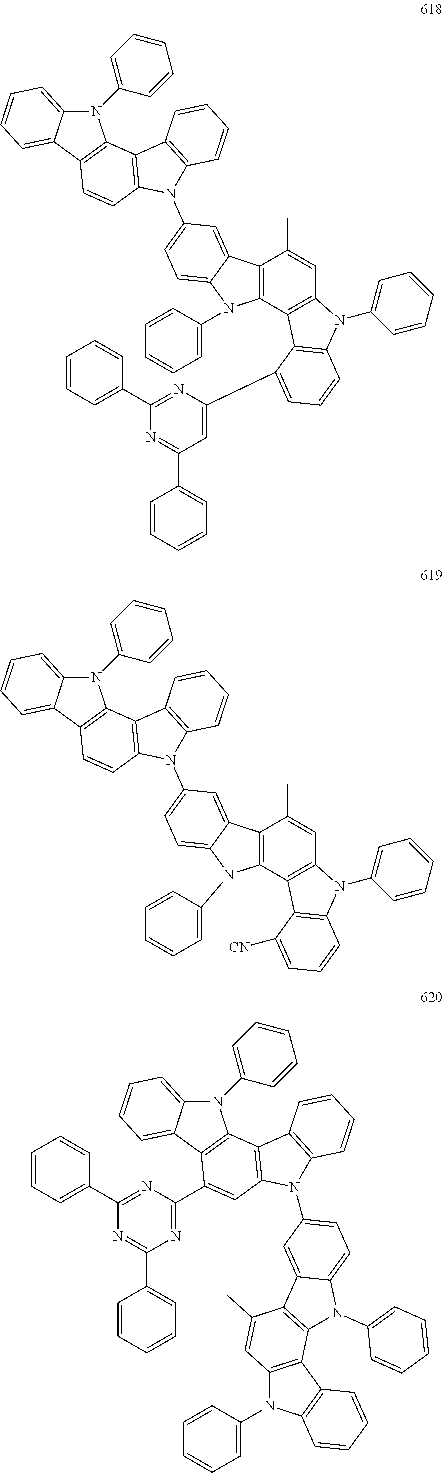

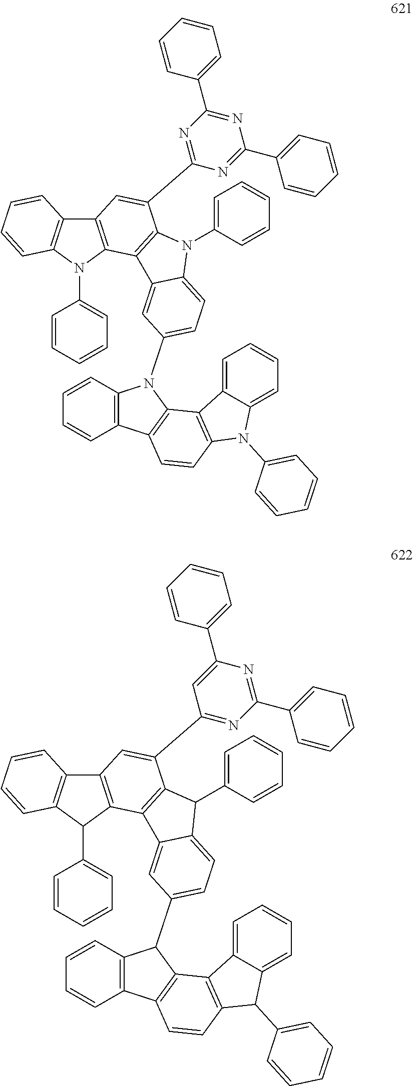 [SMILES: c1ccc(-c2nc(-c3ccccc3)nc(-c3cc4c5ccccc5n(-c5ccccc5)c4c4c5cc(-n6c7ccccc7c7ccc8c(c9ccccc9n8-c8ccccc8)c76)ccc5n(-c5ccccc5)c34)n2)cc1 ; c1ccc(-c2cc(-c3cc4c(c5c3C(c3ccccc3)c3ccc(C6c7ccccc7-c7ccc8c(c76)-c6ccccc6C8c6ccccc6)cc3-5)C(c3ccccc3)c3ccccc3-4)nc(-c3ccccc3)n2)cc1]

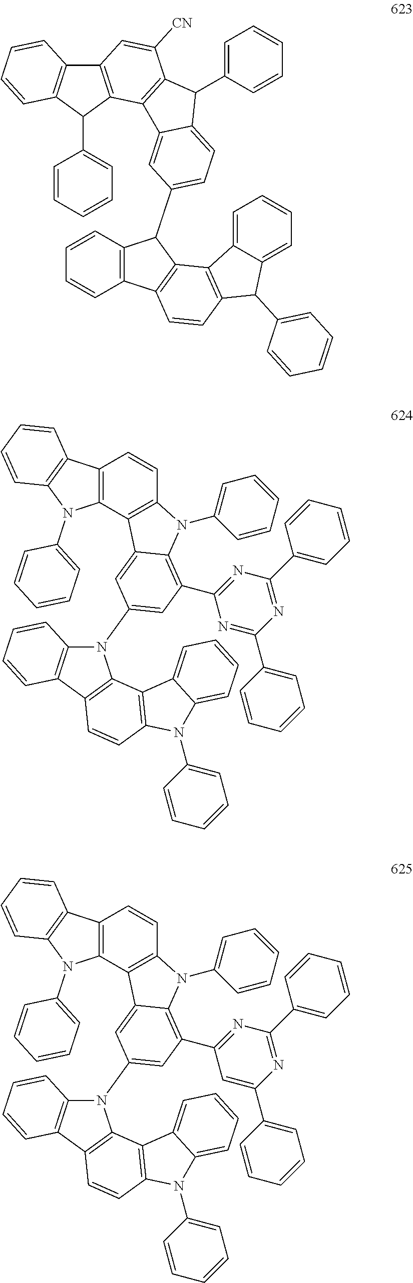 [SMILES: N#Cc1cc2c(c3c1C(c1ccccc1)c1ccc(C4c5ccccc5-c5ccc6c(c54)-c4ccccc4C6c4ccccc4)cc1-3)C(c1ccccc1)c1ccccc1-2 ; c1ccc(-c2nc(-c3ccccc3)nc(-c3cc(-n4c5ccccc5c5ccc6c(c7ccccc7n6-c6ccccc6)c54)cc4c5c(ccc6c7ccccc7n(-c7ccccc7)c65)n(-c5ccccc5)c34)n2)cc1 ; c1ccc(-c2cc(-c3cc(-n4c5ccccc5c5ccc6c(c7ccccc7n6-c6ccccc6)c54)cc4c5c(ccc6c7ccccc7n(-c7ccccc7)c65)n(-c5ccccc5)c34)nc(-c3ccccc3)n2)cc1]

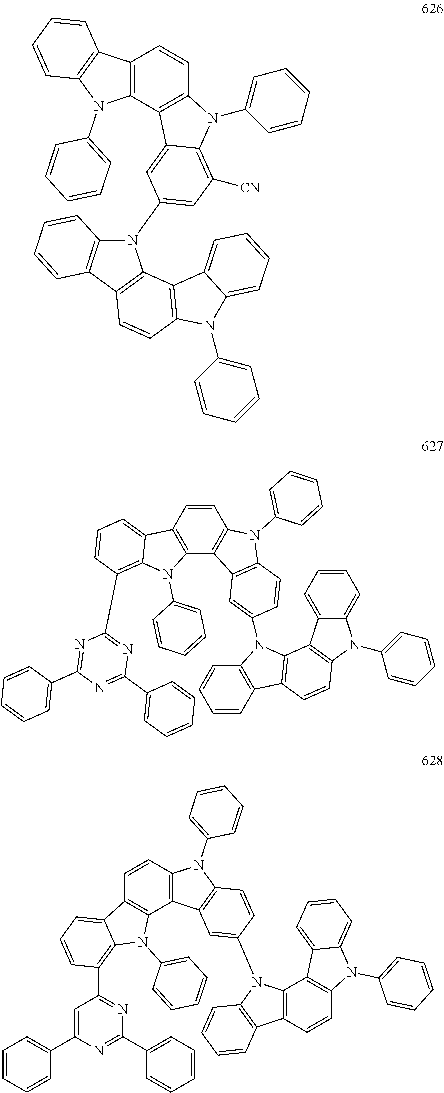 [SMILES: N#Cc1cc(-n2c3ccccc3c3ccc4c(c5ccccc5n4-c4ccccc4)c32)cc2c3c(ccc4c5ccccc5n(-c5ccccc5)c43)n(-c3ccccc3)c12 ; c1ccc(-c2nc(-c3ccccc3)nc(-c3cccc4c5ccc6c(c7cc(-n8c9ccccc9c9ccc%10c(c%11ccccc%11n%10-c%10ccccc%10)c98)ccc7n6-c6ccccc6)c5n(-c5ccccc5)c34)n2)cc1 ; c1ccc(-c2cc(-c3cccc4c5ccc6c(c7cc(-n8c9ccccc9c9ccc%10c(c%11ccccc%11n%10-c%10ccccc%10)c98)ccc7n6-c6ccccc6)c5n(-c5ccccc5)c34)nc(-c3ccccc3)n2)cc1]

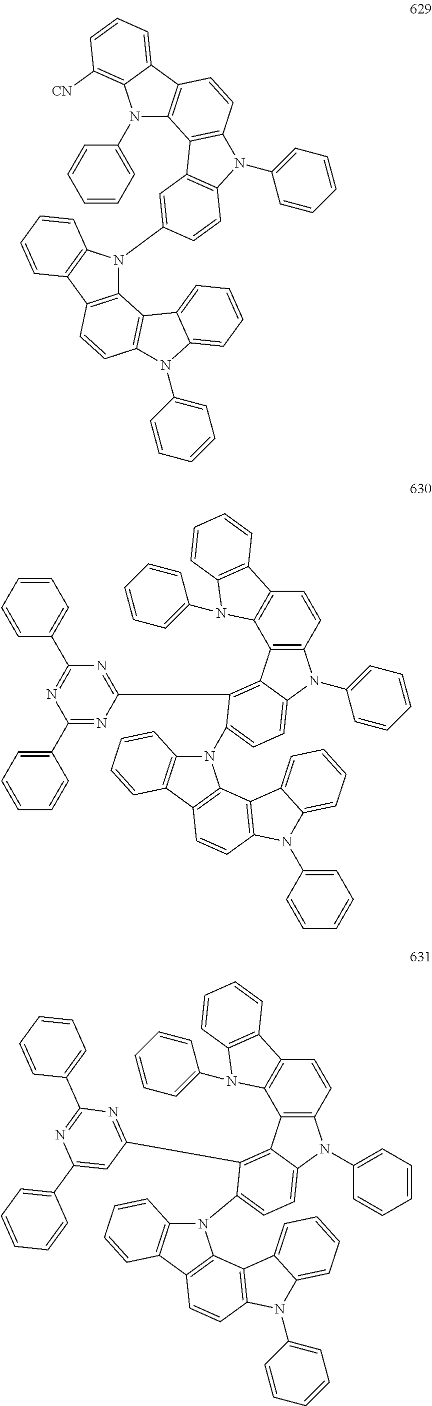

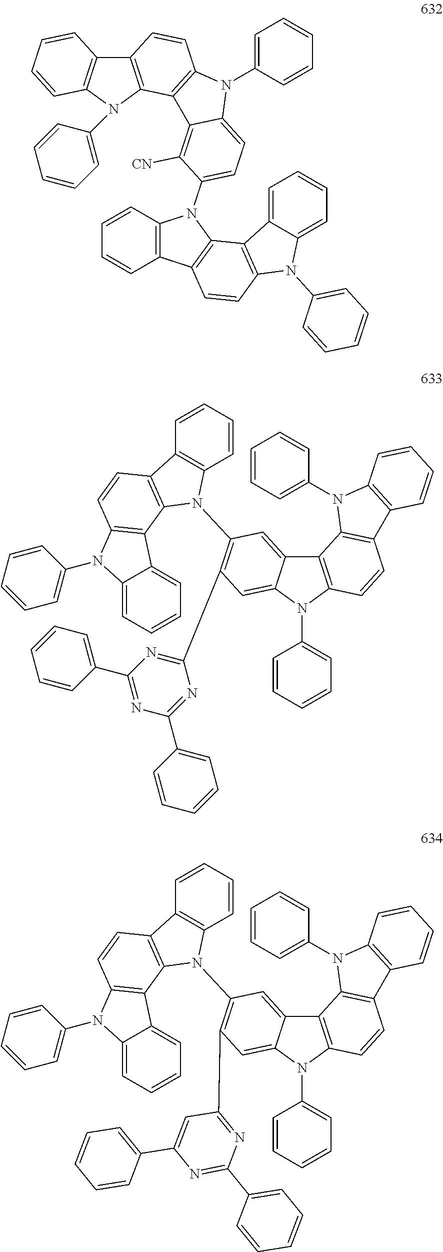

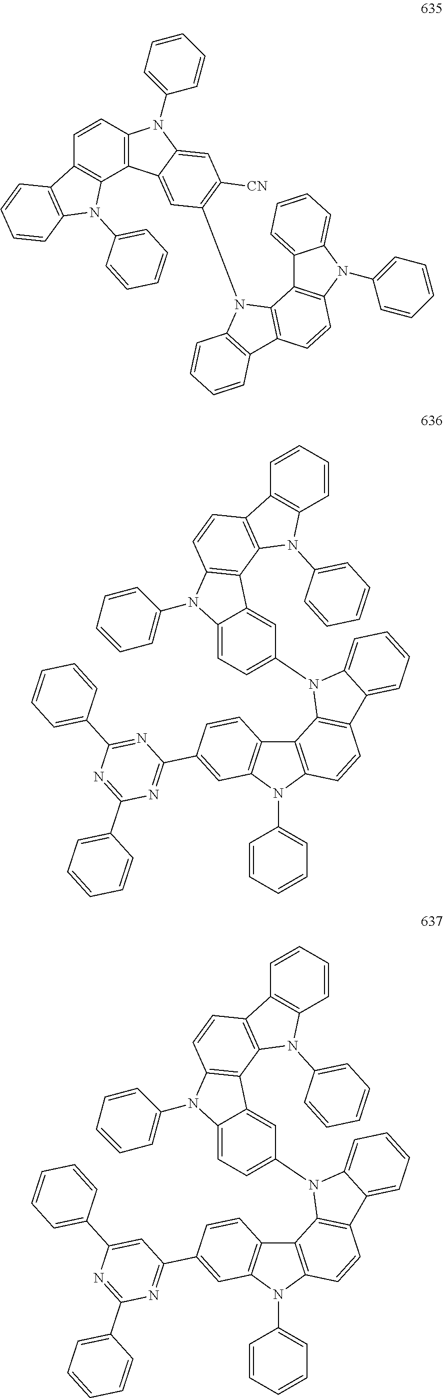 [SMILES: N#Cc1cc2c(cc1-n1c3ccccc3c3ccc4c(c5ccccc5n4-c4ccccc4)c31)c1c(ccc3c4ccccc4n(-c4ccccc4)c31)n2-c1ccccc1 ; c1ccc(-c2nc(-c3ccccc3)nc(-c3ccc4c5c(ccc6c7ccccc7n(-c7ccc8c(c7)c7c(ccc9c%10ccccc%10n(-c%10ccccc%10)c97)n8-c7ccccc7)c65)n(-c5ccccc5)c4c3)n2)cc1 ; c1ccc(-c2cc(-c3ccc4c5c(ccc6c7ccccc7n(-c7ccc8c(c7)c7c(ccc9c%10ccccc%10n(-c%10ccccc%10)c97)n8-c7ccccc7)c65)n(-c5ccccc5)c4c3)nc(-c3ccccc3)n2)cc1]

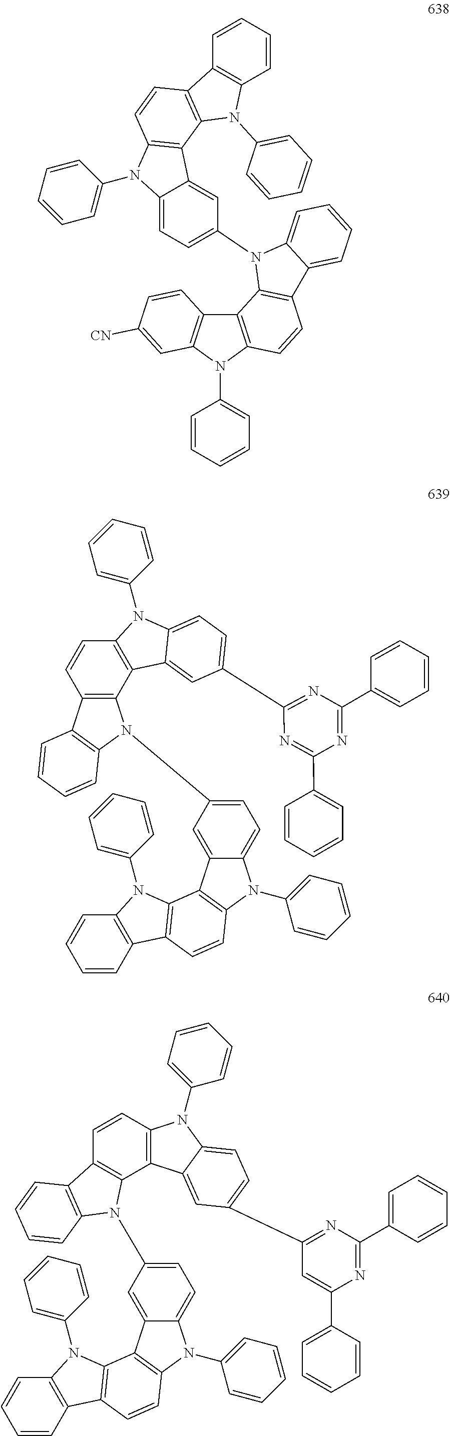

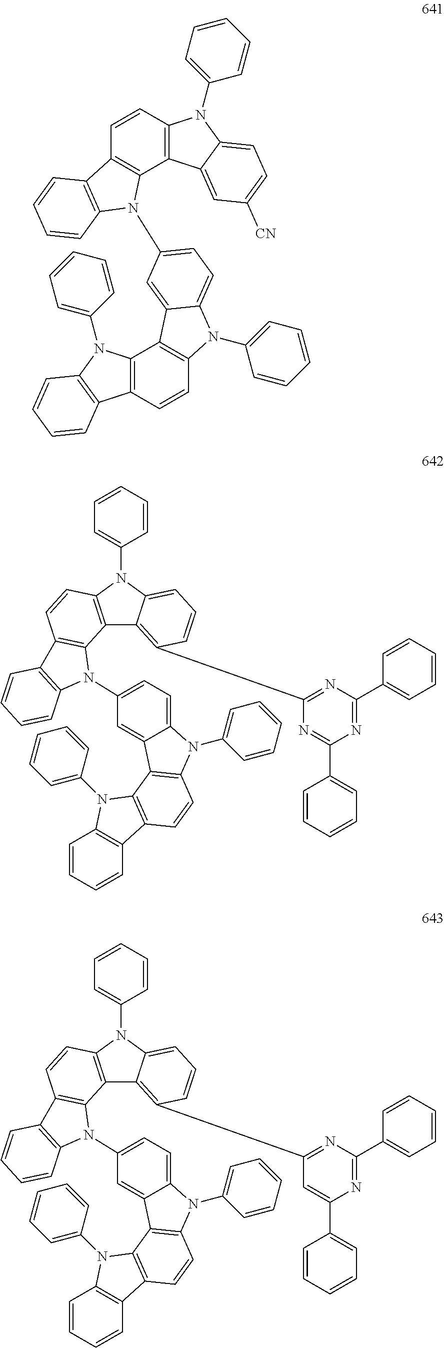 [SMILES: N#Cc1ccc2c(c1)c1c(ccc3c4ccccc4n(-c4ccc5c(c4)c4c(ccc6c7ccccc7n(-c7ccccc7)c64)n5-c4ccccc4)c31)n2-c1ccccc1 ; c1ccc(-c2nc(-c3ccccc3)nc(-c3cccc4c3c3c(ccc5c6ccccc6n(-c6ccc7c(c6)c6c(ccc8c9ccccc9n(-c9ccccc9)c86)n7-c6ccccc6)c53)n4-c3ccccc3)n2)cc1 ; c1ccc(-c2cc(-c3cccc4c3c3c(ccc5c6ccccc6n(-c6ccc7c(c6)c6c(ccc8c9ccccc9n(-c9ccccc9)c86)n7-c6ccccc6)c53)n4-c3ccccc3)nc(-c3ccccc3)n2)cc1]

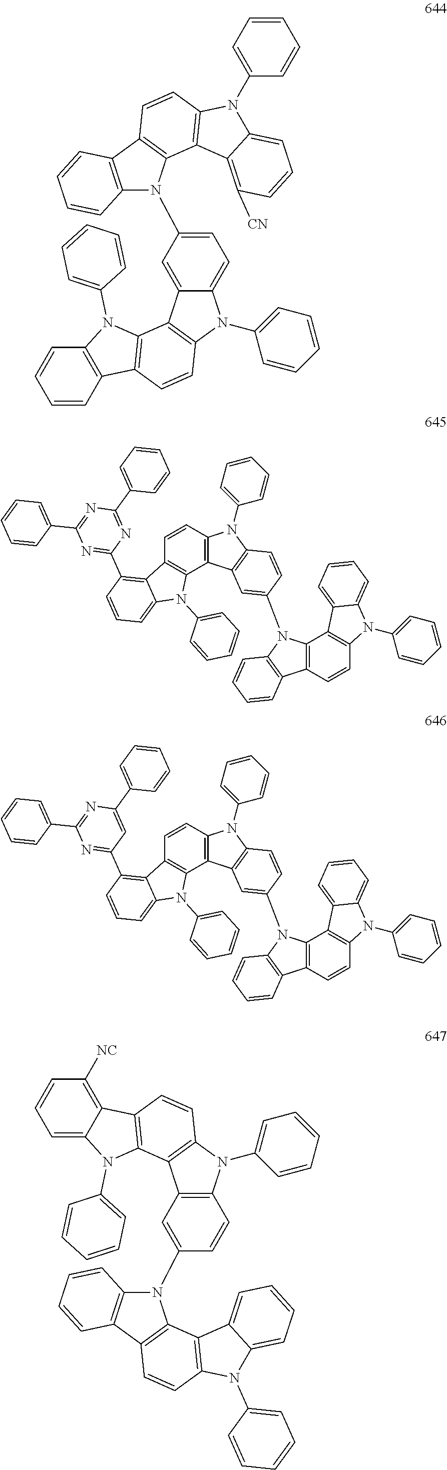

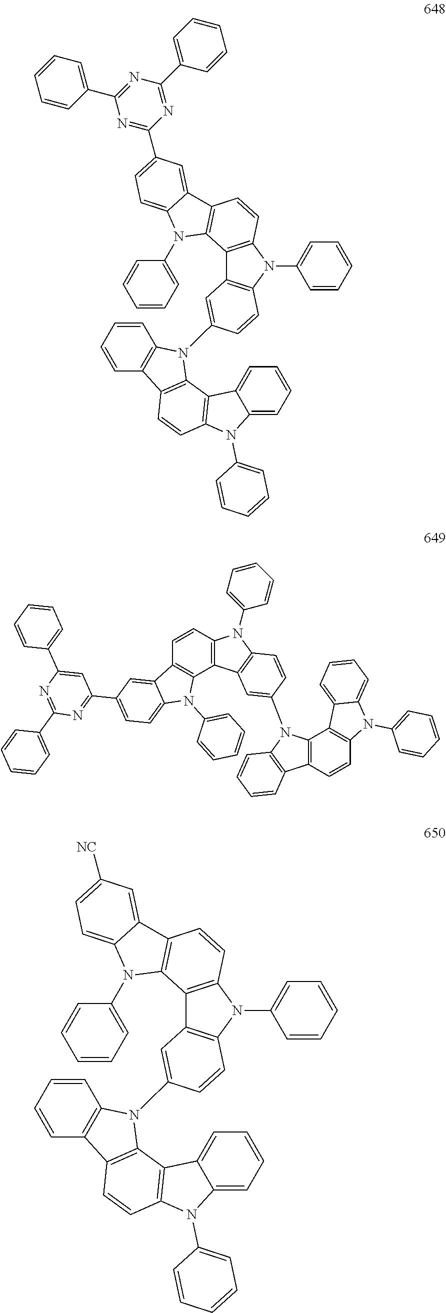 [SMILES: c1ccc(-c2nc(-c3ccccc3)nc(-c3ccc4c(c3)c3ccc5c(c6cc(-n7c8ccccc8c8ccc9c(c%10ccccc%10n9-c9ccccc9)c87)ccc6n5-c5ccccc5)c3n4-c3ccccc3)n2)cc1 ; c1ccc(-c2cc(-c3ccc4c(c3)c3ccc5c(c6cc(-n7c8ccccc8c8ccc9c(c%10ccccc%10n9-c9ccccc9)c87)ccc6n5-c5ccccc5)c3n4-c3ccccc3)nc(-c3ccccc3)n2)cc1 ; N#Cc1ccc2c(c1)c1ccc3c(c4cc(-n5c6ccccc6c6ccc7c(c8ccccc8n7-c7ccccc7)c65)ccc4n3-c3ccccc3)c1n2-c1ccccc1]

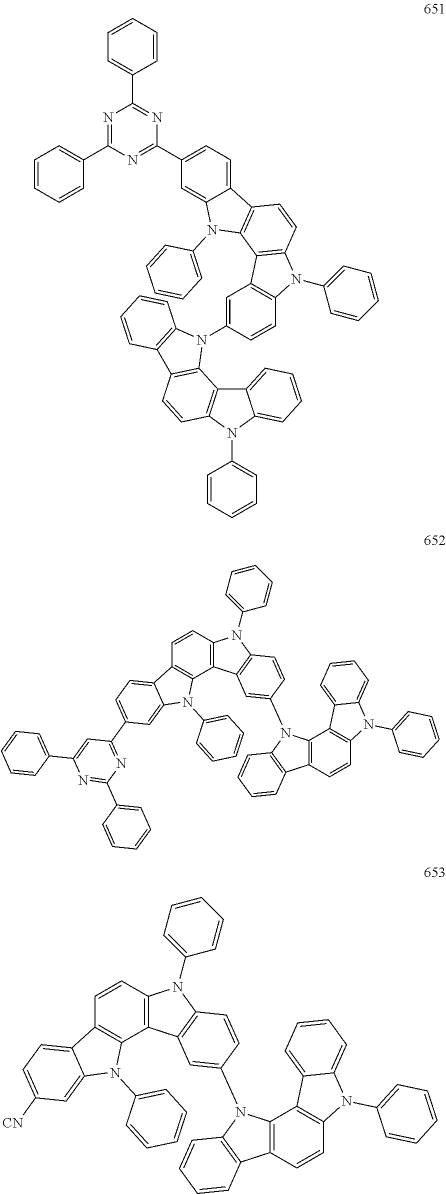

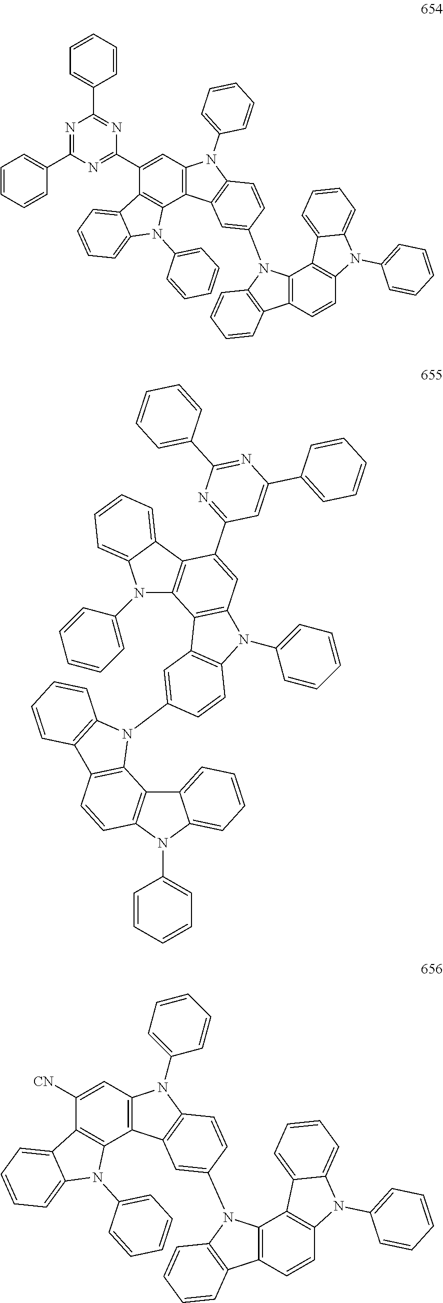

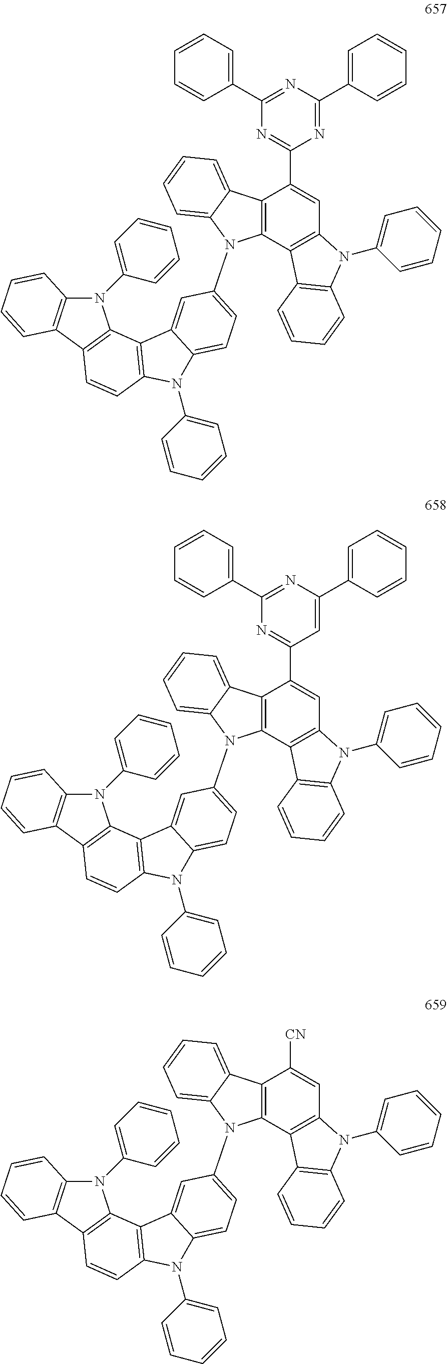 [SMILES: c1ccc(-c2nc(-c3ccccc3)nc(-c3cc4c(c5ccccc5n4-c4ccccc4)c4c3c3ccccc3n4-c3ccc4c(c3)c3c(ccc5c6ccccc6n(-c6ccccc6)c53)n4-c3ccccc3)n2)cc1 ; c1ccc(-c2cc(-c3cc4c(c5ccccc5n4-c4ccccc4)c4c3c3ccccc3n4-c3ccc4c(c3)c3c(ccc5c6ccccc6n(-c6ccccc6)c53)n4-c3ccccc3)nc(-c3ccccc3)n2)cc1 ; N#Cc1cc2c(c3ccccc3n2-c2ccccc2)c2c1c1ccccc1n2-c1ccc2c(c1)c1c(ccc3c4ccccc4n(-c4ccccc4)c31)n2-c1ccccc1]

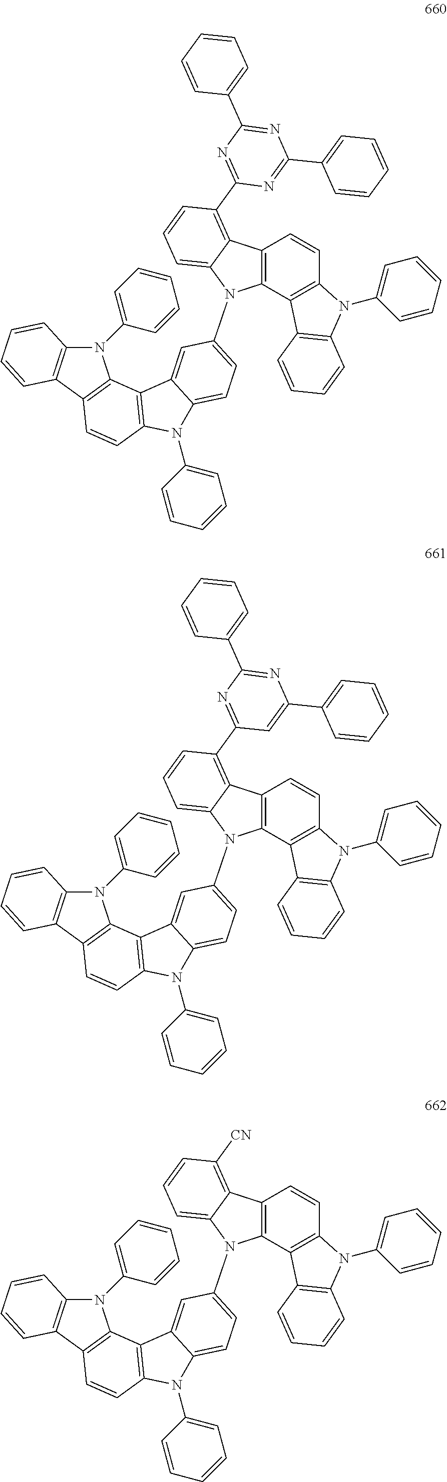 [SMILES: c1ccc(-c2nc(-c3ccccc3)nc(-c3cccc4c3c3ccc5c(c6ccccc6n5-c5ccccc5)c3n4-c3ccc4c(c3)c3c(ccc5c6ccccc6n(-c6ccccc6)c53)n4-c3ccccc3)n2)cc1 ; c1ccc(-c2cc(-c3cccc4c3c3ccc5c(c6ccccc6n5-c5ccccc5)c3n4-c3ccc4c(c3)c3c(ccc5c6ccccc6n(-c6ccccc6)c53)n4-c3ccccc3)nc(-c3ccccc3)n2)cc1 ; N#Cc1cccc2c1c1ccc3c(c4ccccc4n3-c3ccccc3)c1n2-c1ccc2c(c1)c1c(ccc3c4ccccc4n(-c4ccccc4)c31)n2-c1ccccc1]

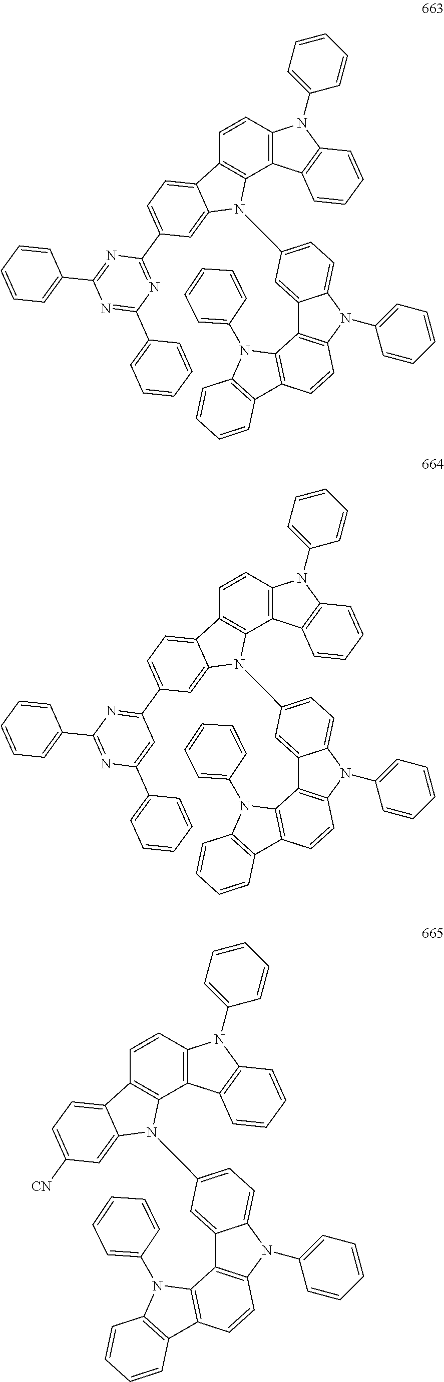

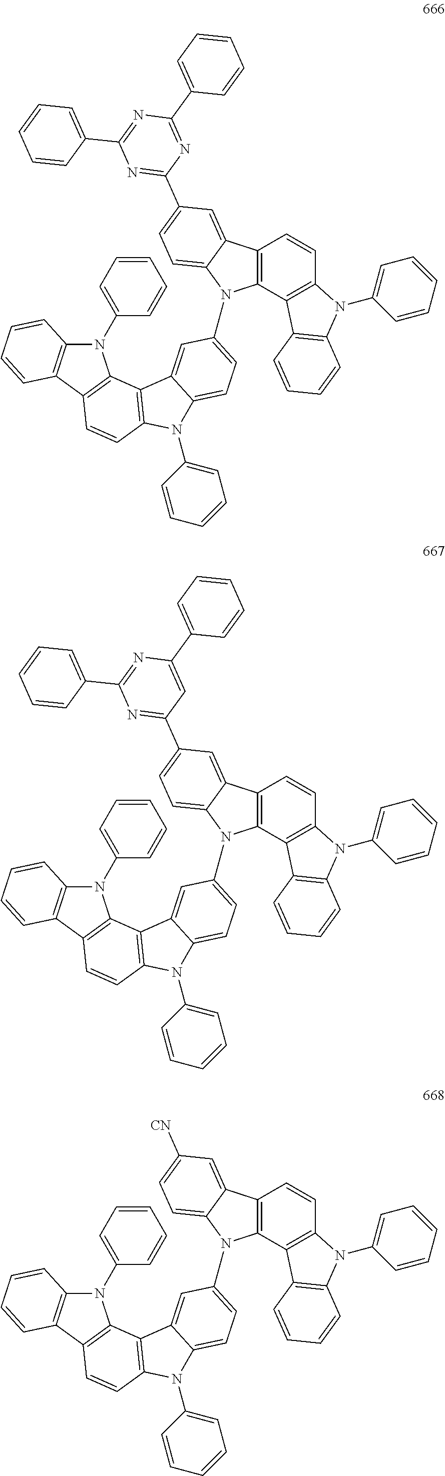

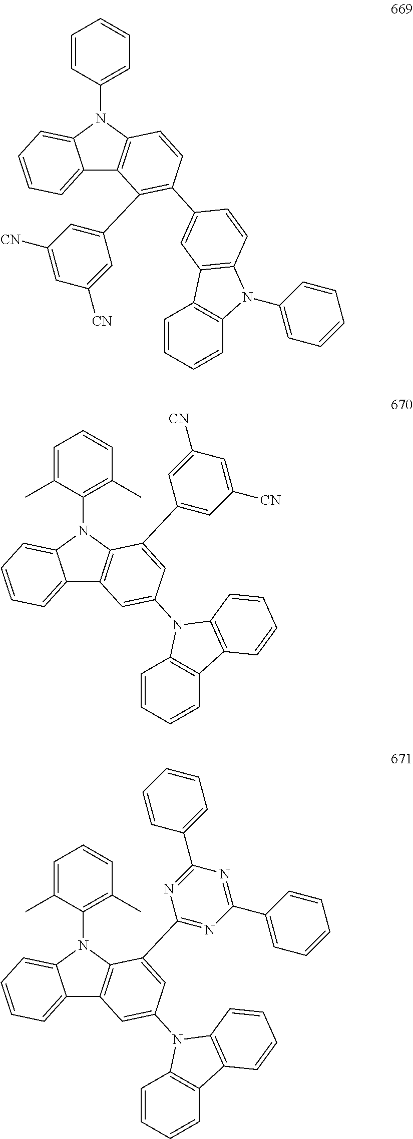

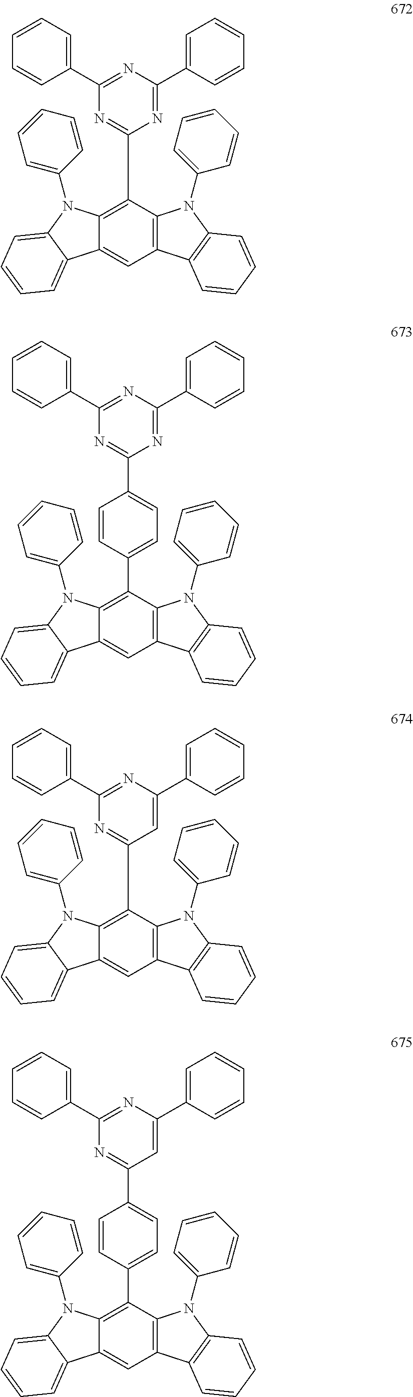 [SMILES: c1ccc(-c2nc(-c3ccccc3)nc(-c3c4c(cc5c6ccccc6n(-c6ccccc6)c35)c3ccccc3n4-c3ccccc3)n2)cc1 ; c1ccc(-c2nc(-c3ccccc3)nc(-c3ccc(-c4c5c(cc6c7ccccc7n(-c7ccccc7)c46)c4ccccc4n5-c4ccccc4)cc3)n2)cc1 ; c1ccc(-c2cc(-c3c4c(cc5c6ccccc6n(-c6ccccc6)c35)c3ccccc3n4-c3ccccc3)nc(-c3ccccc3)n2)cc1 ; c1ccc(-c2cc(-c3ccc(-c4c5c(cc6c7ccccc7n(-c7ccccc7)c46)c4ccccc4n5-c4ccccc4)cc3)nc(-c3ccccc3)n2)cc1]

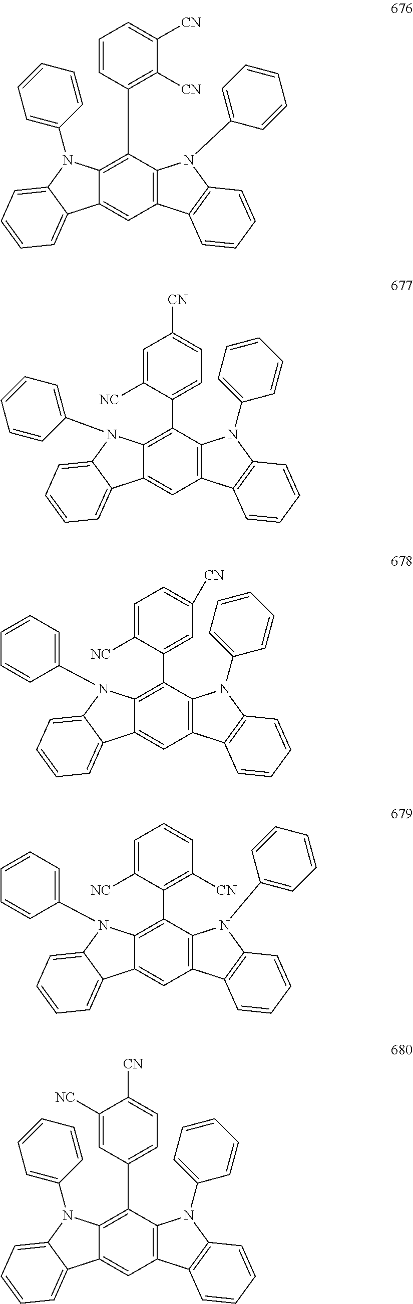 [SMILES: N#Cc1cccc(-c2c3c(cc4c5ccccc5n(-c5ccccc5)c24)c2ccccc2n3-c2ccccc2)c1C#N ; N#Cc1ccc(-c2c3c(cc4c5ccccc5n(-c5ccccc5)c24)c2ccccc2n3-c2ccccc2)c(C#N)c1 ; N#Cc1ccc(C#N)c(-c2c3c(cc4c5ccccc5n(-c5ccccc5)c24)c2ccccc2n3-c2ccccc2)c1 ; N#Cc1cccc(C#N)c1-c1c2c(cc3c4ccccc4n(-c4ccccc4)c13)c1ccccc1n2-c1ccccc1 ; N#Cc1ccc(-c2c3c(cc4c5ccccc5n(-c5ccccc5)c24)c2ccccc2n3-c2ccccc2)cc1C#N]

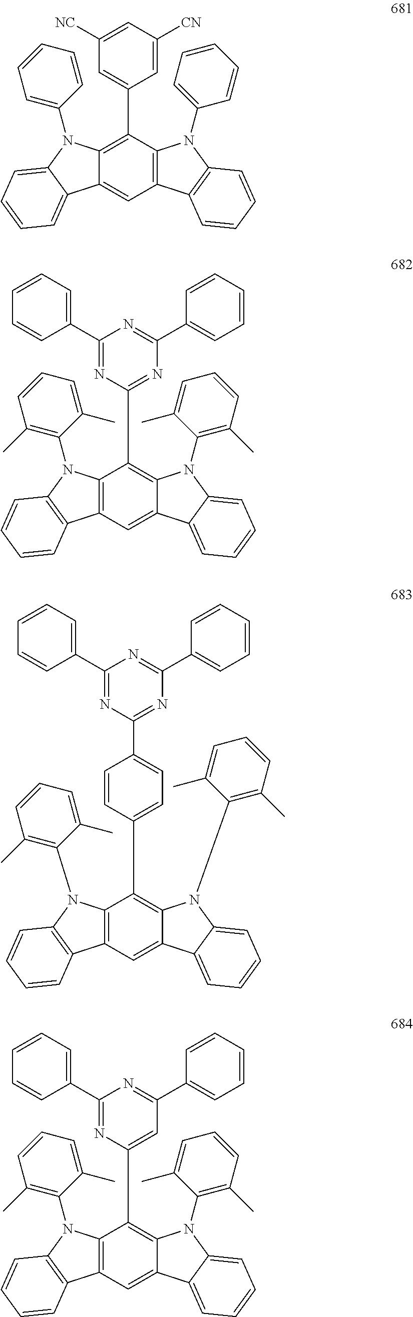

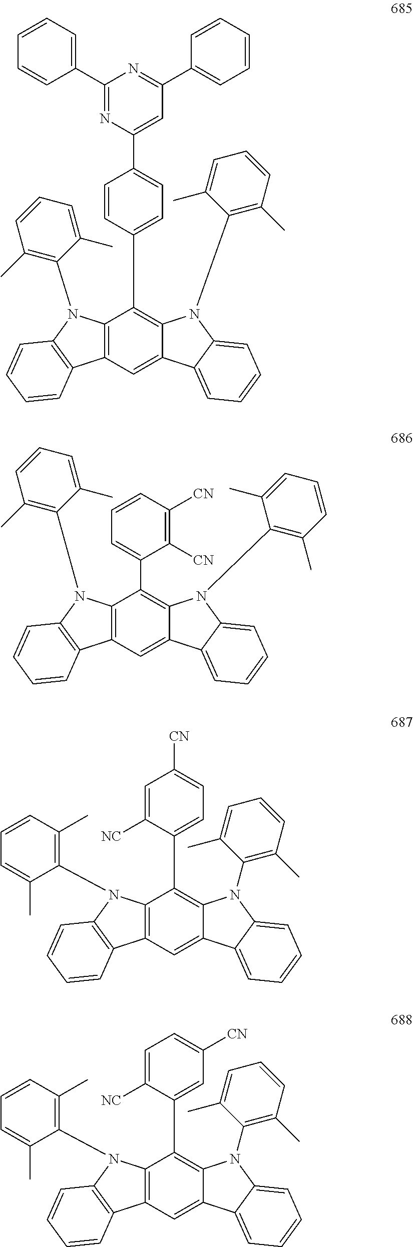

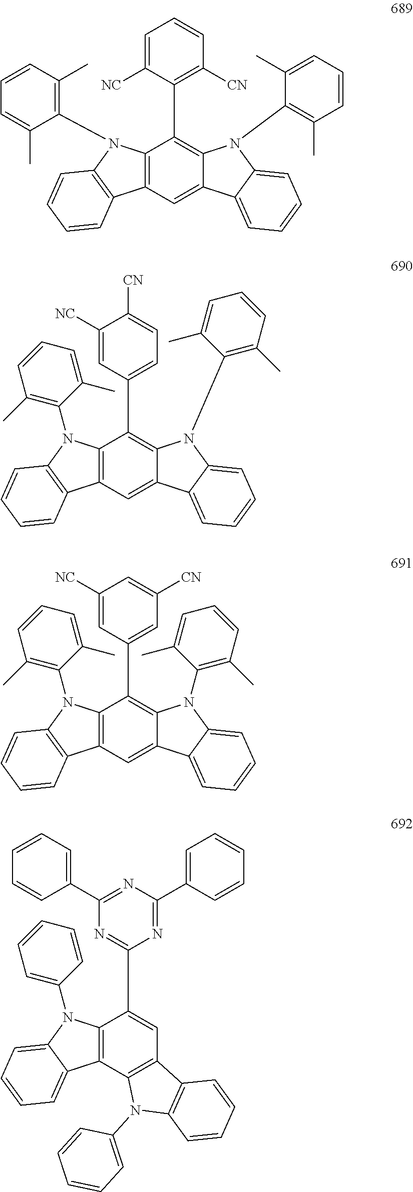 [SMILES: Cc1cccc(C)c1-n1c2ccccc2c2cc3c4ccccc4n(-c4c(C)cccc4C)c3c(-c3c(C#N)cccc3C#N)c21 ; Cc1cccc(C)c1-n1c2ccccc2c2cc3c4ccccc4n(-c4c(C)cccc4C)c3c(-c3ccc(C#N)c(C#N)c3)c21 ; Cc1cccc(C)c1-n1c2ccccc2c2cc3c4ccccc4n(-c4c(C)cccc4C)c3c(-c3cc(C#N)cc(C#N)c3)c21 ; c1ccc(-c2nc(-c3ccccc3)nc(-c3cc4c5ccccc5n(-c5ccccc5)c4c4c5ccccc5n(-c5ccccc5)c34)n2)cc1]

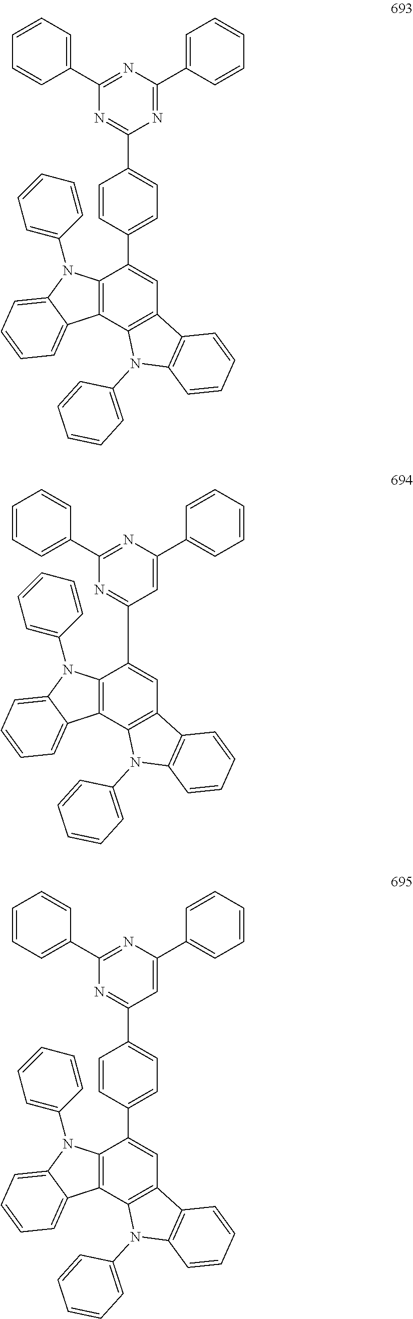 [SMILES: c1ccc(-c2nc(-c3ccccc3)nc(-c3ccc(-c4cc5c6ccccc6n(-c6ccccc6)c5c5c6ccccc6n(-c6ccccc6)c45)cc3)n2)cc1 ; c1ccc(-c2cc(-c3cc4c5ccccc5n(-c5ccccc5)c4c4c5ccccc5n(-c5ccccc5)c34)nc(-c3ccccc3)n2)cc1 ; c1ccc(-c2cc(-c3ccc(-c4cc5c6ccccc6n(-c6ccccc6)c5c5c6ccccc6n(-c6ccccc6)c45)cc3)nc(-c3ccccc3)n2)cc1]

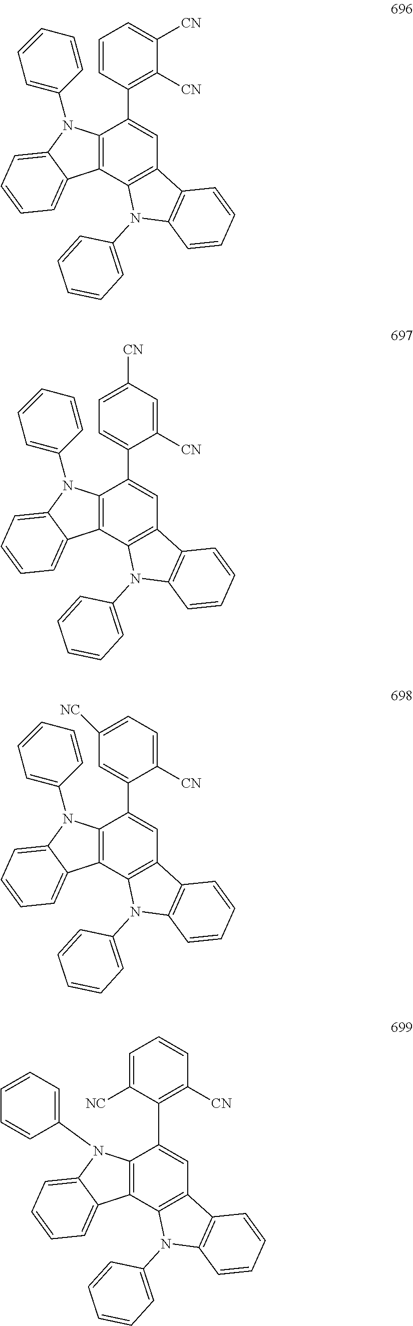 [SMILES: N#Cc1cccc(-c2cc3c4ccccc4n(-c4ccccc4)c3c3c4ccccc4n(-c4ccccc4)c23)c1C#N ; N#Cc1ccc(-c2cc3c4ccccc4n(-c4ccccc4)c3c3c4ccccc4n(-c4ccccc4)c23)c(C#N)c1 ; N#Cc1ccc(C#N)c(-c2cc3c4ccccc4n(-c4ccccc4)c3c3c4ccccc4n(-c4ccccc4)c23)c1 ; N#Cc1cccc(C#N)c1-c1cc2c3ccccc3n(-c3ccccc3)c2c2c3ccccc3n(-c3ccccc3)c12]

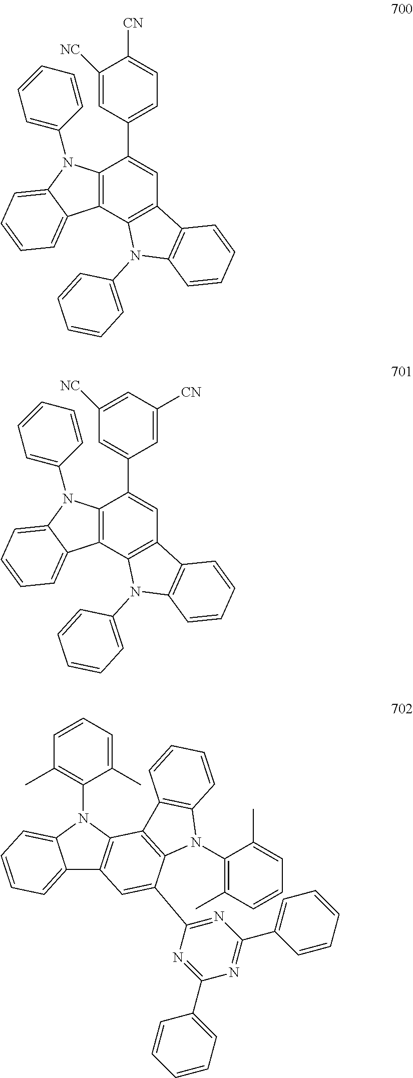 [SMILES: N#Cc1ccc(-c2cc3c4ccccc4n(-c4ccccc4)c3c3c4ccccc4n(-c4ccccc4)c23)cc1C#N ; N#Cc1cc(C#N)cc(-c2cc3c4ccccc4n(-c4ccccc4)c3c3c4ccccc4n(-c4ccccc4)c23)c1 ; Cc1cccc(C)c1-n1c2ccccc2c2c1c(-c1nc(-c3ccccc3)nc(-c3ccccc3)n1)cc1c3ccccc3n(-c3c(C)cccc3C)c12]

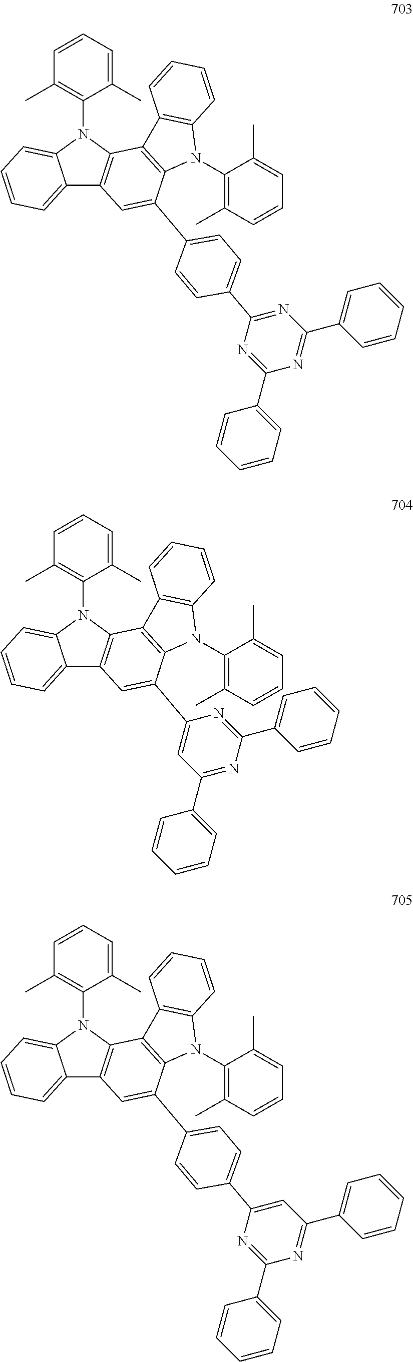 [SMILES: Cc1cccc(C)c1-n1c2ccccc2c2c1c(-c1ccc(-c3nc(-c4ccccc4)nc(-c4ccccc4)n3)cc1)cc1c3ccccc3n(-c3c(C)cccc3C)c12 ; Cc1cccc(C)c1-n1c2ccccc2c2c1c(-c1cc(-c3ccccc3)nc(-c3ccccc3)n1)cc1c3ccccc3n(-c3c(C)cccc3C)c12 ; Cc1cccc(C)c1-n1c2ccccc2c2c1c(-c1ccc(-c3cc(-c4ccccc4)nc(-c4ccccc4)n3)cc1)cc1c3ccccc3n(-c3c(C)cccc3C)c12]

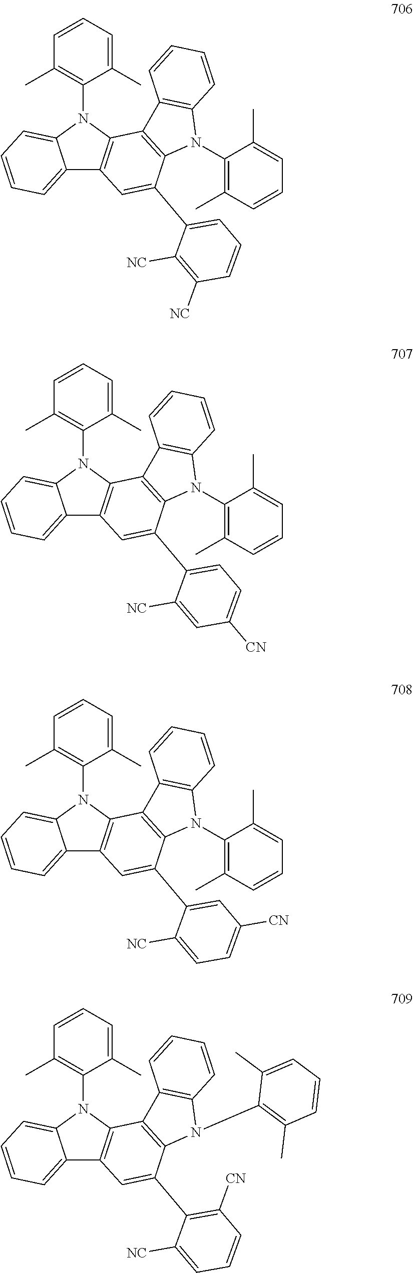 [SMILES: Cc1cccc(C)c1-n1c2ccccc2c2c1c(-c1cccc(C#N)c1C#N)cc1c3ccccc3n(-c3c(C)cccc3C)c12 ; Cc1cccc(C)c1-n1c2ccccc2c2c1c(-c1ccc(C#N)cc1C#N)cc1c3ccccc3n(-c3c(C)cccc3C)c12 ; Cc1cccc(C)c1-n1c2ccccc2c2c1c(-c1cc(C#N)ccc1C#N)cc1c3ccccc3n(-c3c(C)cccc3C)c12 ; Cc1cccc(C)c1-n1c2ccccc2c2c1c(-c1c(C#N)cccc1C#N)cc1c3ccccc3n(-c3c(C)cccc3C)c12]

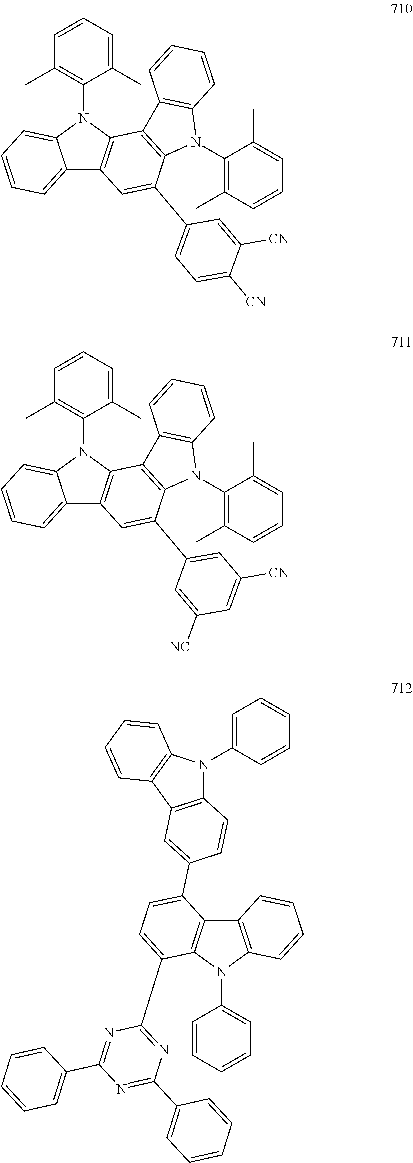 [SMILES: Cc1cccc(C)c1-n1c2ccccc2c2c1c(-c1ccc(C#N)c(C#N)c1)cc1c3ccccc3n(-c3c(C)cccc3C)c12 ; Cc1cccc(C)c1-n1c2ccccc2c2c1c(-c1cc(C#N)cc(C#N)c1)cc1c3ccccc3n(-c3c(C)cccc3C)c12 ; c1ccc(-c2nc(-c3ccccc3)nc(-c3ccc(-c4ccc5c(c4)c4ccccc4n5-c4ccccc4)c4c5ccccc5n(-c5ccccc5)c34)n2)cc1]

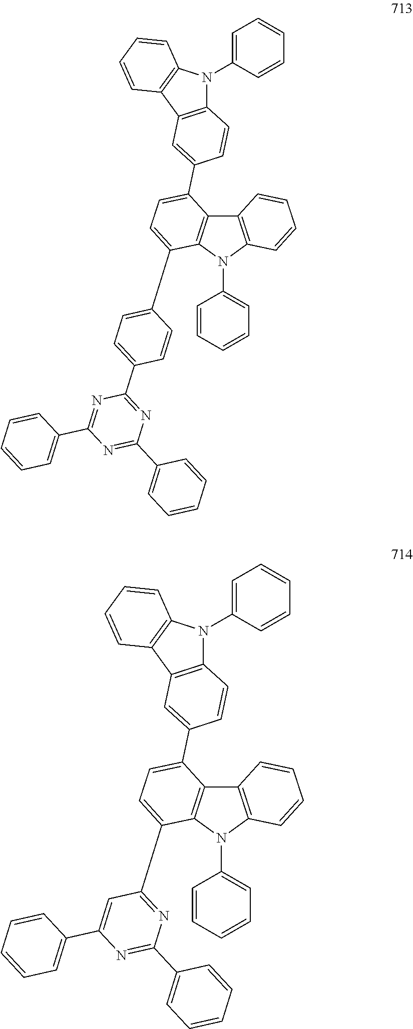 [SMILES: c1ccc(-c2nc(-c3ccccc3)nc(-c3ccc(-c4ccc(-c5ccc6c(c5)c5ccccc5n6-c5ccccc5)c5c6ccccc6n(-c6ccccc6)c45)cc3)n2)cc1 ; c1ccc(-c2cc(-c3ccc(-c4ccc5c(c4)c4ccccc4n5-c4ccccc4)c4c5ccccc5n(-c5ccccc5)c34)nc(-c3ccccc3)n2)cc1]

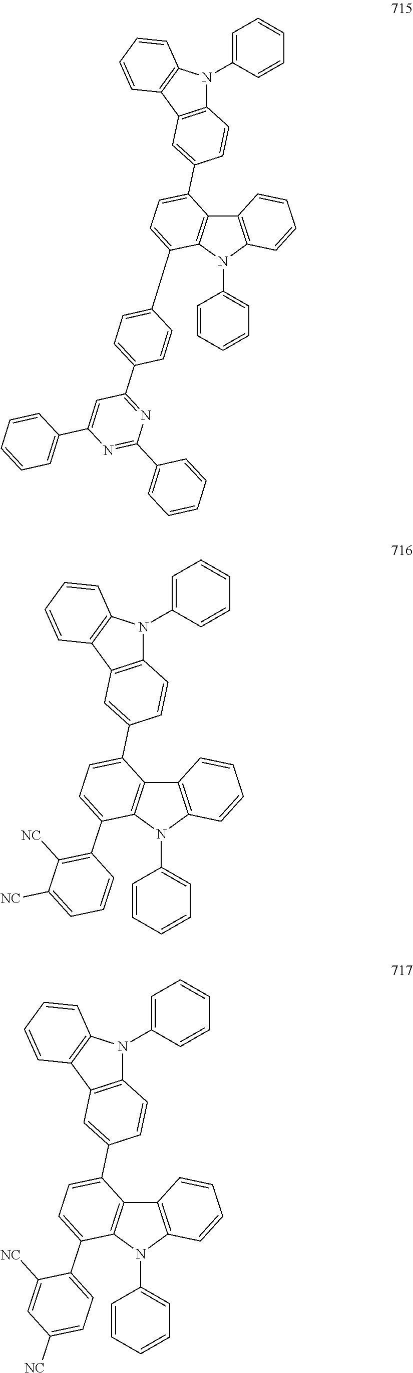 [SMILES: c1ccc(-c2cc(-c3ccc(-c4ccc(-c5ccc6c(c5)c5ccccc5n6-c5ccccc5)c5c6ccccc6n(-c6ccccc6)c45)cc3)nc(-c3ccccc3)n2)cc1 ; N#Cc1cccc(-c2ccc(-c3ccc4c(c3)c3ccccc3n4-c3ccccc3)c3c4ccccc4n(-c4ccccc4)c23)c1C#N ; N#Cc1ccc(-c2ccc(-c3ccc4c(c3)c3ccccc3n4-c3ccccc3)c3c4ccccc4n(-c4ccccc4)c23)c(C#N)c1]

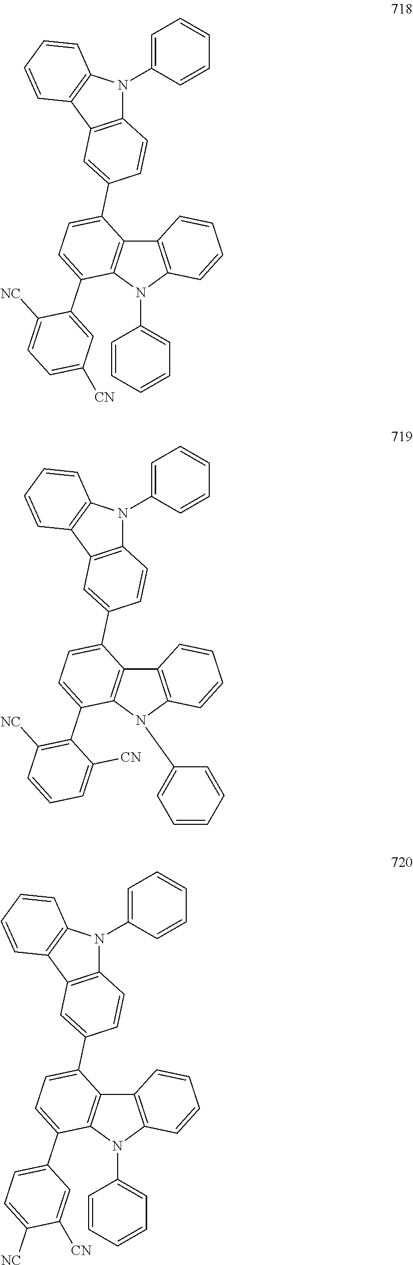 [SMILES: N#Cc1ccc(C#N)c(-c2ccc(-c3ccc4c(c3)c3ccccc3n4-c3ccccc3)c3c4ccccc4n(-c4ccccc4)c23)c1 ; N#Cc1cccc(C#N)c1-c1ccc(-c2ccc3c(c2)c2ccccc2n3-c2ccccc2)c2c3ccccc3n(-c3ccccc3)c12 ; N#Cc1ccc(-c2ccc(-c3ccc4c(c3)c3ccccc3n4-c3ccccc3)c3c4ccccc4n(-c4ccccc4)c23)cc1C#N]

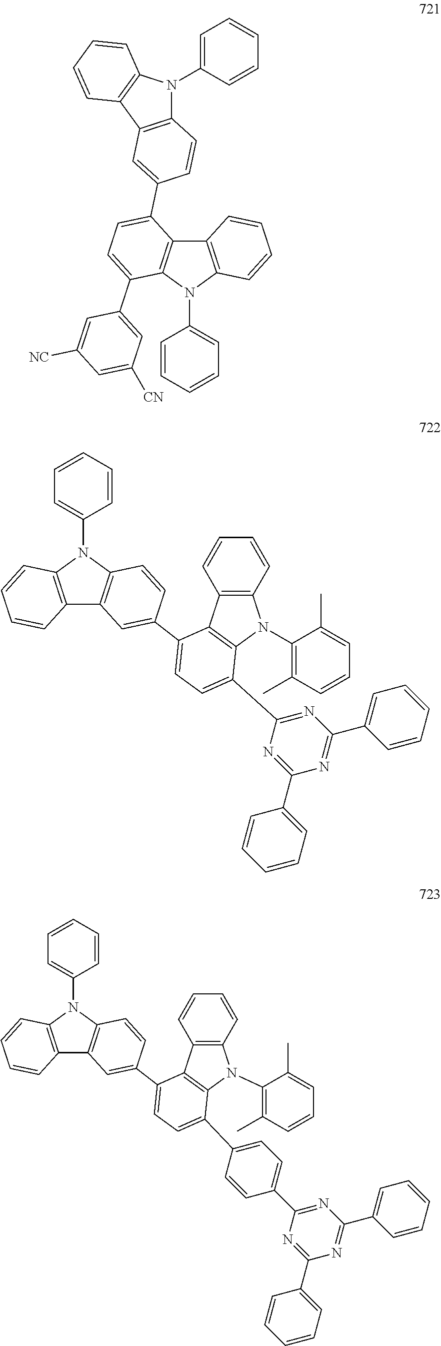 [SMILES: N#Cc1cc(C#N)cc(-c2ccc(-c3ccc4c(c3)c3ccccc3n4-c3ccccc3)c3c4ccccc4n(-c4ccccc4)c23)c1 ; Cc1cccc(C)c1-n1c2ccccc2c2c(-c3ccc4c(c3)c3ccccc3n4-c3ccccc3)ccc(-c3nc(-c4ccccc4)nc(-c4ccccc4)n3)c21 ; Cc1cccc(C)c1-n1c2ccccc2c2c(-c3ccc4c(c3)c3ccccc3n4-c3ccccc3)ccc(-c3ccc(-c4nc(-c5ccccc5)nc(-c5ccccc5)n4)cc3)c21]

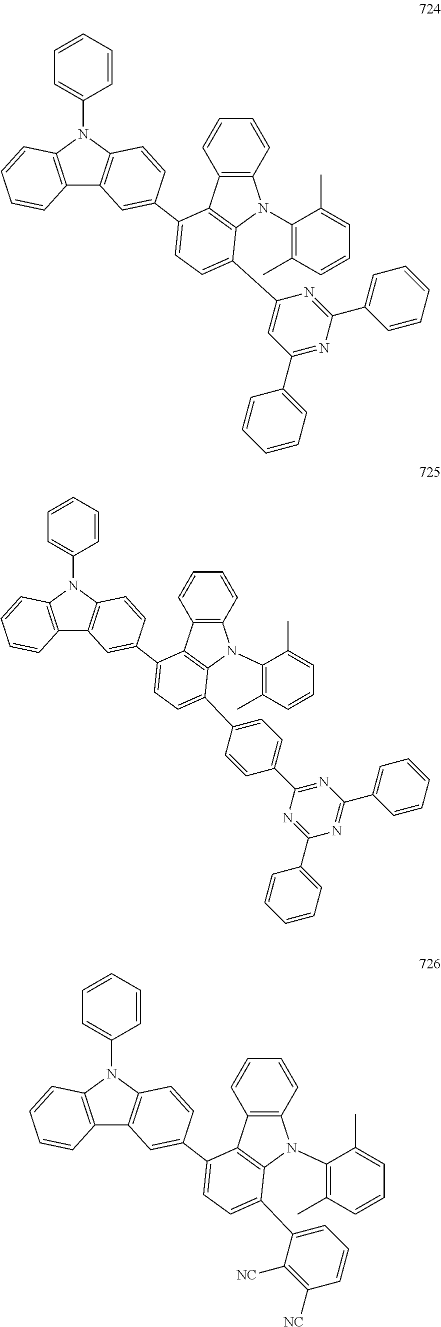 [SMILES: Cc1cccc(C)c1-n1c2ccccc2c2c(-c3ccc4c(c3)c3ccccc3n4-c3ccccc3)ccc(-c3cc(-c4ccccc4)nc(-c4ccccc4)n3)c21 ; Cc1cccc(C)c1-n1c2ccccc2c2c(-c3ccc4c(c3)c3ccccc3n4-c3ccccc3)ccc(-c3ccc(-c4nc(-c5ccccc5)nc(-c5ccccc5)n4)cc3)c21 ; Cc1cccc(C)c1-n1c2ccccc2c2c(-c3ccc4c(c3)c3ccccc3n4-c3ccccc3)ccc(-c3cccc(C#N)c3C#N)c21]

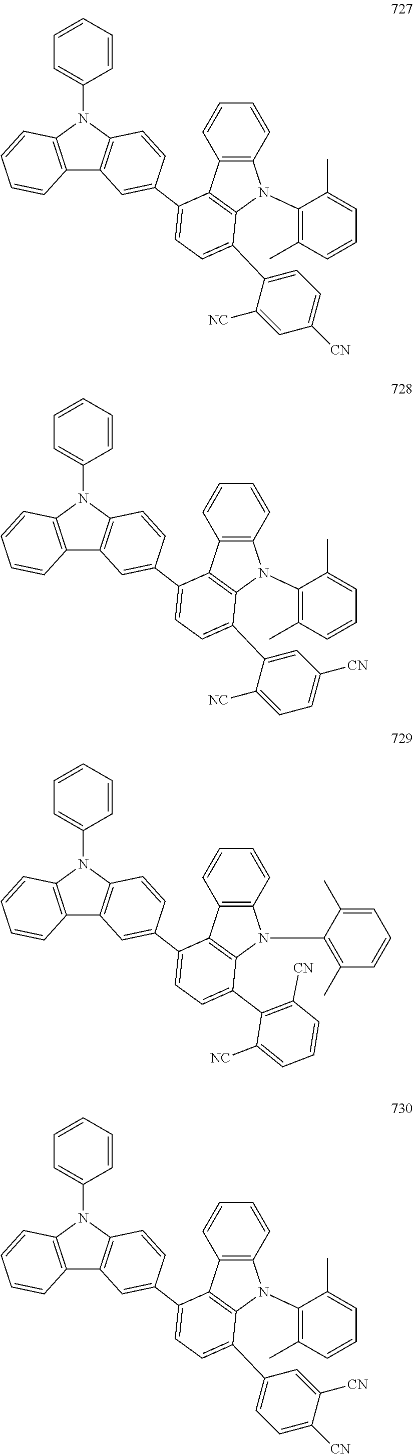 [SMILES: Cc1cccc(C)c1-n1c2ccccc2c2c(-c3ccc4c(c3)c3ccccc3n4-c3ccccc3)ccc(-c3ccc(C#N)cc3C#N)c21 ; Cc1cccc(C)c1-n1c2ccccc2c2c(-c3ccc4c(c3)c3ccccc3n4-c3ccccc3)ccc(-c3cc(C#N)ccc3C#N)c21 ; Cc1cccc(C)c1-n1c2ccccc2c2c(-c3ccc4c(c3)c3ccccc3n4-c3ccccc3)ccc(-c3c(C#N)cccc3C#N)c21 ; Cc1cccc(C)c1-n1c2ccccc2c2c(-c3ccc4c(c3)c3ccccc3n4-c3ccccc3)ccc(-c3ccc(C#N)c(C#N)c3)c21]

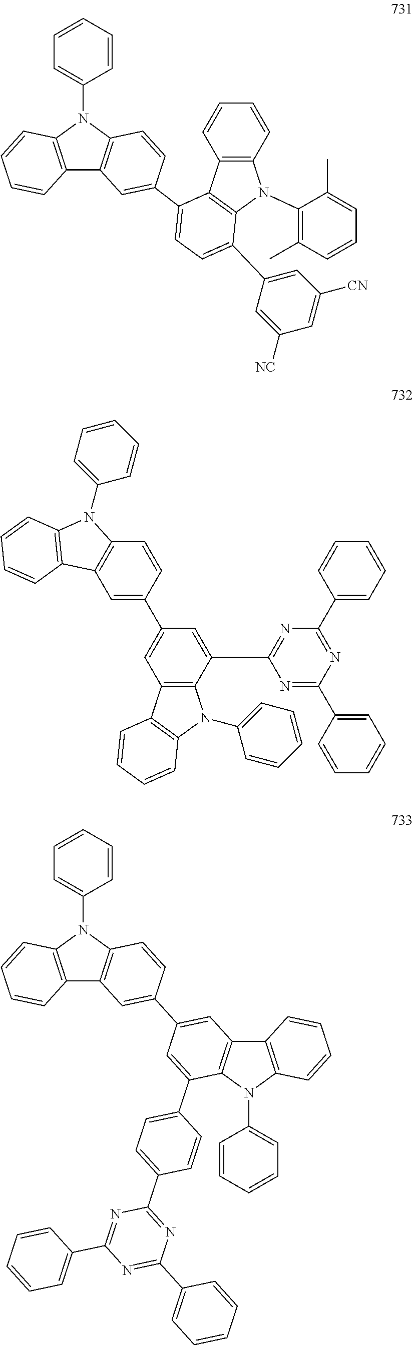 [SMILES: Cc1cccc(C)c1-n1c2ccccc2c2c(-c3ccc4c(c3)c3ccccc3n4-c3ccccc3)ccc(-c3cc(C#N)cc(C#N)c3)c21 ; c1ccc(-c2nc(-c3ccccc3)nc(-c3cc(-c4ccc5c(c4)c4ccccc4n5-c4ccccc4)cc4c5ccccc5n(-c5ccccc5)c34)n2)cc1 ; c1ccc(-c2nc(-c3ccccc3)nc(-c3ccc(-c4cc(-c5ccc6c(c5)c5ccccc5n6-c5ccccc5)cc5c6ccccc6n(-c6ccccc6)c45)cc3)n2)cc1]

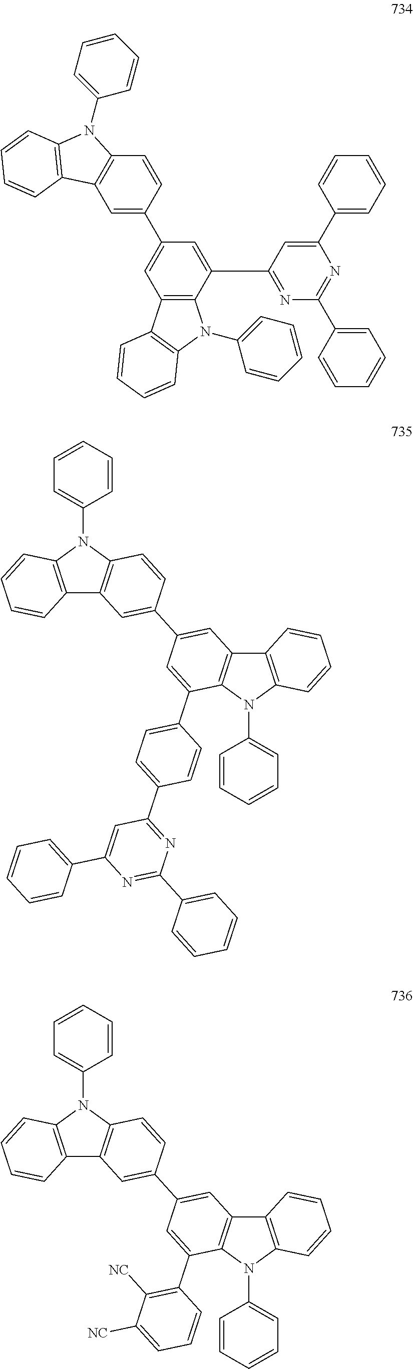 [SMILES: c1ccc(-c2cc(-c3cc(-c4ccc5c(c4)c4ccccc4n5-c4ccccc4)cc4c5ccccc5n(-c5ccccc5)c34)nc(-c3ccccc3)n2)cc1 ; c1ccc(-c2cc(-c3ccc(-c4cc(-c5ccc6c(c5)c5ccccc5n6-c5ccccc5)cc5c6ccccc6n(-c6ccccc6)c45)cc3)nc(-c3ccccc3)n2)cc1 ; N#Cc1cccc(-c2cc(-c3ccc4c(c3)c3ccccc3n4-c3ccccc3)cc3c4ccccc4n(-c4ccccc4)c23)c1C#N]

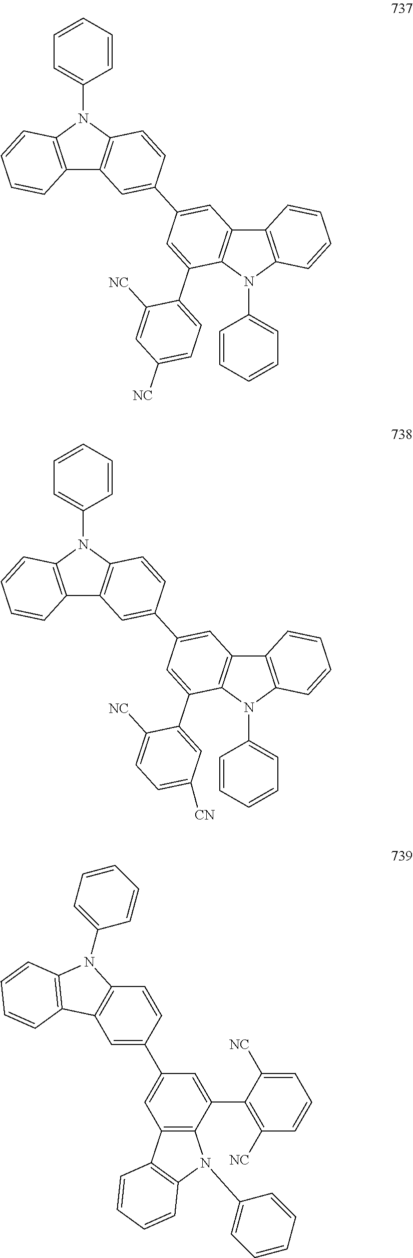 [SMILES: N#Cc1ccc(-c2cc(-c3ccc4c(c3)c3ccccc3n4-c3ccccc3)cc3c4ccccc4n(-c4ccccc4)c23)c(C#N)c1 ; N#Cc1ccc(C#N)c(-c2cc(-c3ccc4c(c3)c3ccccc3n4-c3ccccc3)cc3c4ccccc4n(-c4ccccc4)c23)c1 ; N#Cc1cccc(C#N)c1-c1cc(-c2ccc3c(c2)c2ccccc2n3-c2ccccc2)cc2c3ccccc3n(-c3ccccc3)c12]

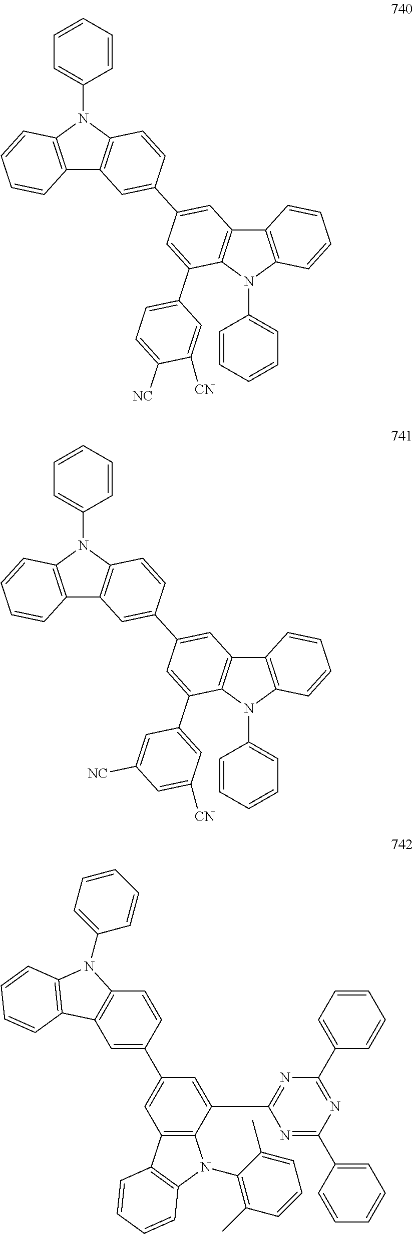 [SMILES: N#Cc1ccc(-c2cc(-c3ccc4c(c3)c3ccccc3n4-c3ccccc3)cc3c4ccccc4n(-c4ccccc4)c23)cc1C#N ; N#Cc1cc(C#N)cc(-c2cc(-c3ccc4c(c3)c3ccccc3n4-c3ccccc3)cc3c4ccccc4n(-c4ccccc4)c23)c1 ; Cc1cccc(C)c1-n1c2ccccc2c2cc(-c3ccc4c(c3)c3ccccc3n4-c3ccccc3)cc(-c3nc(-c4ccccc4)nc(-c4ccccc4)n3)c21]

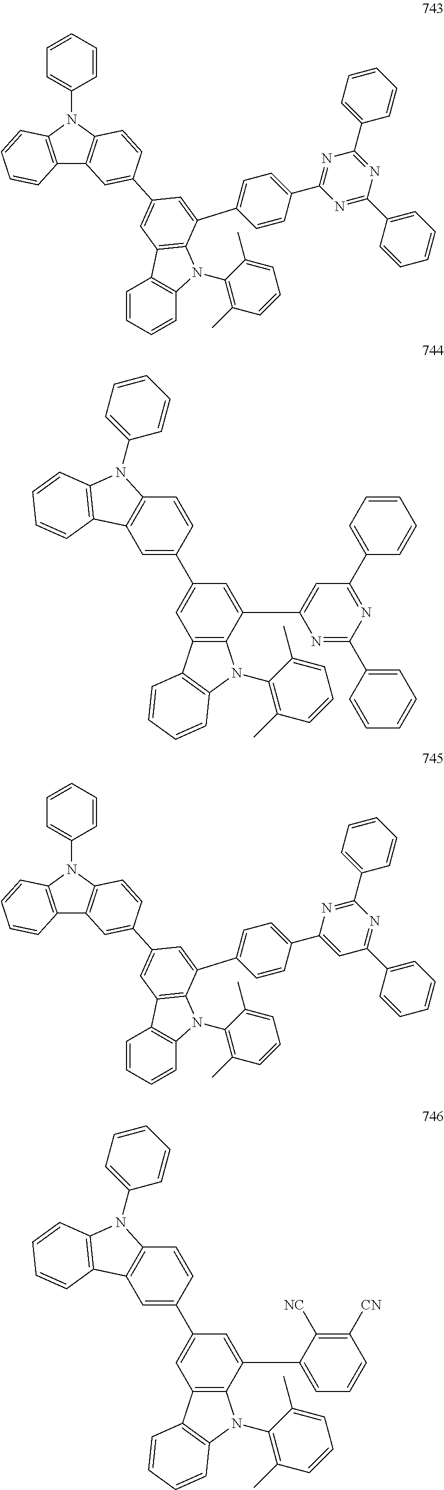 [SMILES: Cc1cccc(C)c1-n1c2ccccc2c2cc(-c3ccc4c(c3)c3ccccc3n4-c3ccccc3)cc(-c3ccc(-c4nc(-c5ccccc5)nc(-c5ccccc5)n4)cc3)c21 ; Cc1cccc(C)c1-n1c2ccccc2c2cc(-c3ccc4c(c3)c3ccccc3n4-c3ccccc3)cc(-c3cc(-c4ccccc4)nc(-c4ccccc4)n3)c21 ; Cc1cccc(C)c1-n1c2ccccc2c2cc(-c3ccc4c(c3)c3ccccc3n4-c3ccccc3)cc(-c3ccc(-c4cc(-c5ccccc5)nc(-c5ccccc5)n4)cc3)c21 ; Cc1cccc(C)c1-n1c2ccccc2c2cc(-c3ccc4c(c3)c3ccccc3n4-c3ccccc3)cc(-c3cccc(C#N)c3C#N)c21]

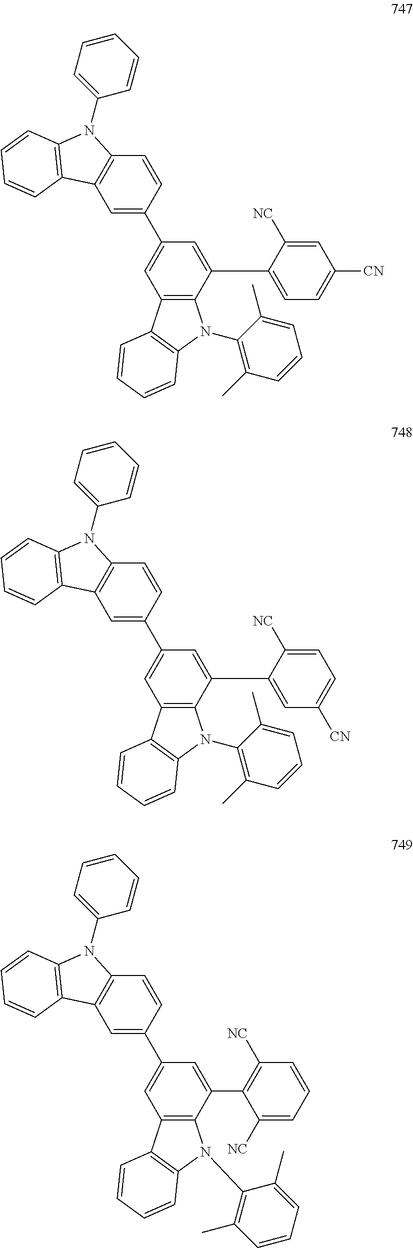 [SMILES: Cc1cccc(C)c1-n1c2ccccc2c2cc(-c3ccc4c(c3)c3ccccc3n4-c3ccccc3)cc(-c3ccc(C#N)cc3C#N)c21 ; Cc1cccc(C)c1-n1c2ccccc2c2cc(-c3ccc4c(c3)c3ccccc3n4-c3ccccc3)cc(-c3cc(C#N)ccc3C#N)c21 ; Cc1cccc(C)c1-n1c2ccccc2c2cc(-c3ccc4c(c3)c3ccccc3n4-c3ccccc3)cc(-c3c(C#N)cccc3C#N)c21]

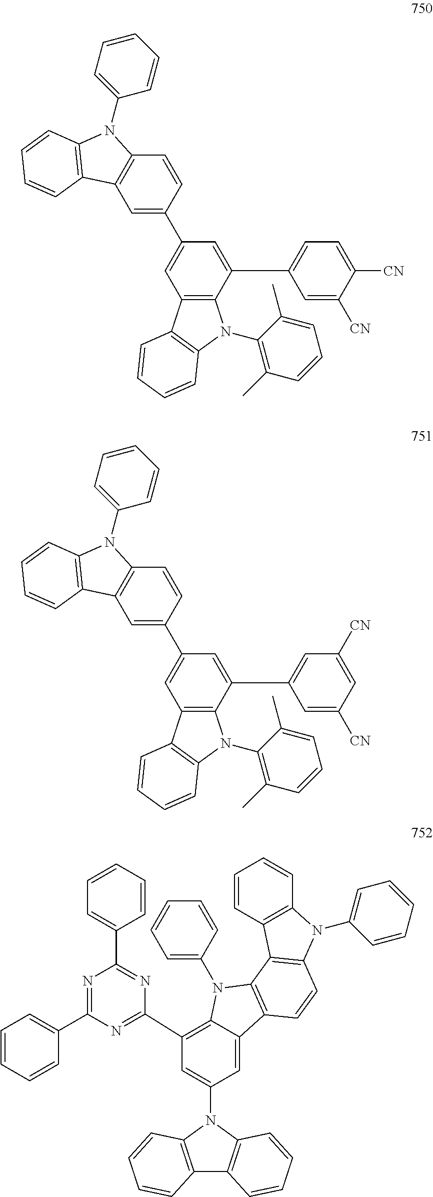 [SMILES: Cc1cccc(C)c1-n1c2ccccc2c2cc(-c3ccc4c(c3)c3ccccc3n4-c3ccccc3)cc(-c3ccc(C#N)c(C#N)c3)c21 ; Cc1cccc(C)c1-n1c2ccccc2c2cc(-c3ccc4c(c3)c3ccccc3n4-c3ccccc3)cc(-c3cc(C#N)cc(C#N)c3)c21 ; c1ccc(-c2nc(-c3ccccc3)nc(-c3cc(-n4c5ccccc5c5ccccc54)cc4c5ccc6c(c7ccccc7n6-c6ccccc6)c5n(-c5ccccc5)c34)n2)cc1]

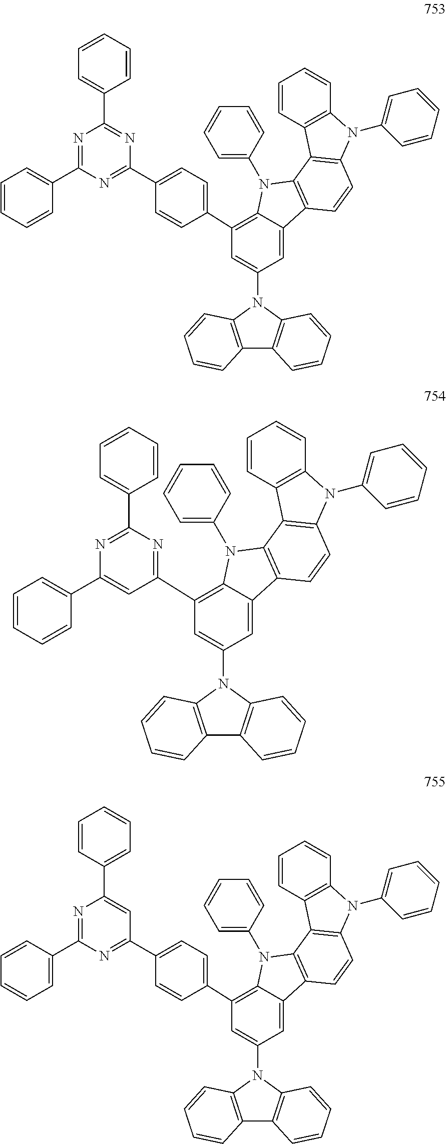 [SMILES: c1ccc(-c2nc(-c3ccccc3)nc(-c3ccc(-c4cc(-n5c6ccccc6c6ccccc65)cc5c6ccc7c(c8ccccc8n7-c7ccccc7)c6n(-c6ccccc6)c45)cc3)n2)cc1 ; c1ccc(-c2cc(-c3cc(-n4c5ccccc5c5ccccc54)cc4c5ccc6c(c7ccccc7n6-c6ccccc6)c5n(-c5ccccc5)c34)nc(-c3ccccc3)n2)cc1 ; c1ccc(-c2cc(-c3ccc(-c4cc(-n5c6ccccc6c6ccccc65)cc5c6ccc7c(c8ccccc8n7-c7ccccc7)c6n(-c6ccccc6)c45)cc3)nc(-c3ccccc3)n2)cc1]

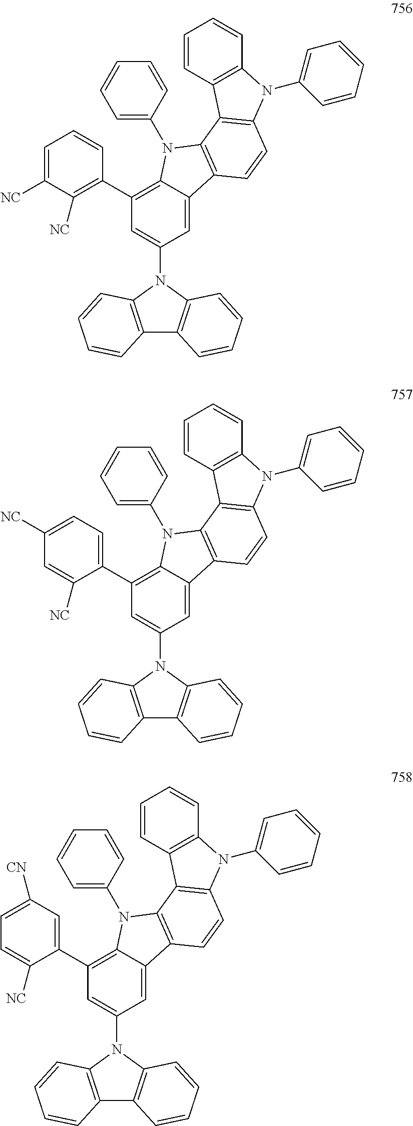

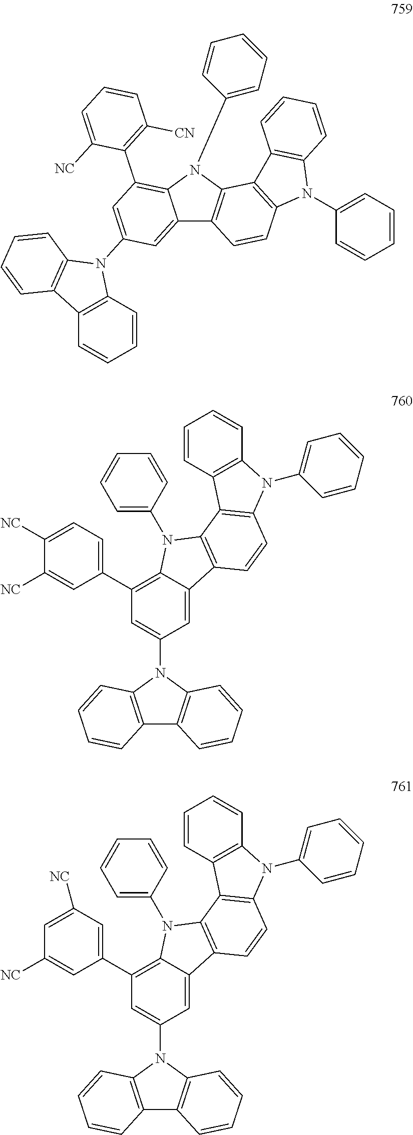 [SMILES: N#Cc1cccc(C#N)c1-c1cc(-n2c3ccccc3c3ccccc32)cc2c3ccc4c(c5ccccc5n4-c4ccccc4)c3n(-c3ccccc3)c12 ; N#Cc1ccc(-c2cc(-n3c4ccccc4c4ccccc43)cc3c4ccc5c(c6ccccc6n5-c5ccccc5)c4n(-c4ccccc4)c23)cc1C#N ; N#Cc1cc(C#N)cc(-c2cc(-n3c4ccccc4c4ccccc43)cc3c4ccc5c(c6ccccc6n5-c5ccccc5)c4n(-c4ccccc4)c23)c1]

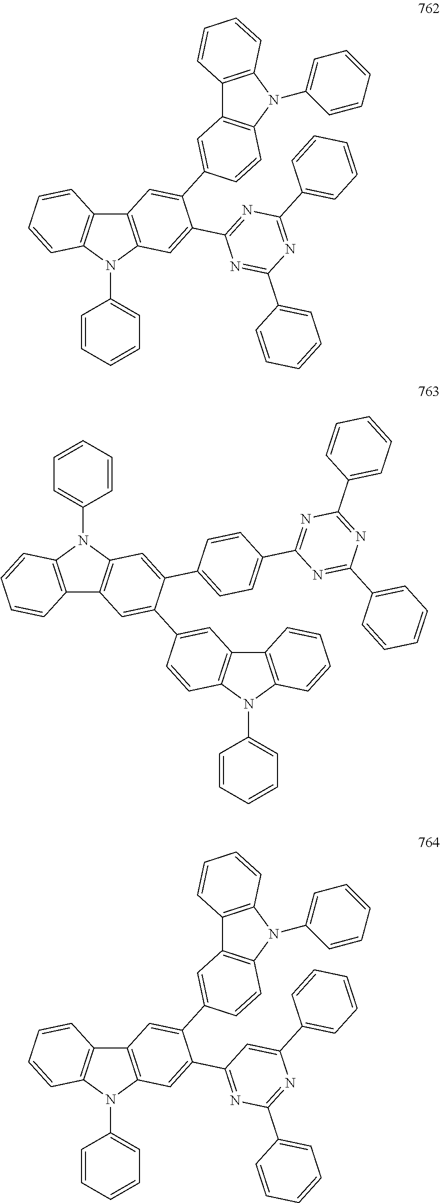 [SMILES: c1ccc(-c2nc(-c3ccccc3)nc(-c3cc4c(cc3-c3ccc5c(c3)c3ccccc3n5-c3ccccc3)c3ccccc3n4-c3ccccc3)n2)cc1 ; c1ccc(-c2nc(-c3ccccc3)nc(-c3ccc(-c4cc5c(cc4-c4ccc6c(c4)c4ccccc4n6-c4ccccc4)c4ccccc4n5-c4ccccc4)cc3)n2)cc1 ; c1ccc(-c2cc(-c3cc4c(cc3-c3ccc5c(c3)c3ccccc3n5-c3ccccc3)c3ccccc3n4-c3ccccc3)nc(-c3ccccc3)n2)cc1]

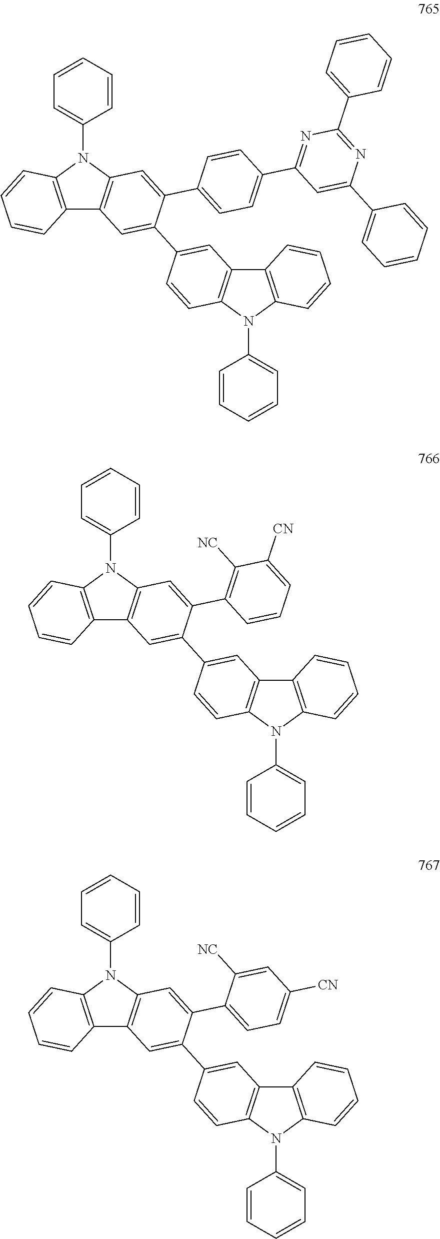 [SMILES: c1ccc(-c2cc(-c3ccc(-c4cc5c(cc4-c4ccc6c(c4)c4ccccc4n6-c4ccccc4)c4ccccc4n5-c4ccccc4)cc3)nc(-c3ccccc3)n2)cc1 ; N#Cc1cccc(-c2cc3c(cc2-c2ccc4c(c2)c2ccccc2n4-c2ccccc2)c2ccccc2n3-c2ccccc2)c1C#N ; N#Cc1ccc(-c2cc3c(cc2-c2ccc4c(c2)c2ccccc2n4-c2ccccc2)c2ccccc2n3-c2ccccc2)c(C#N)c1]

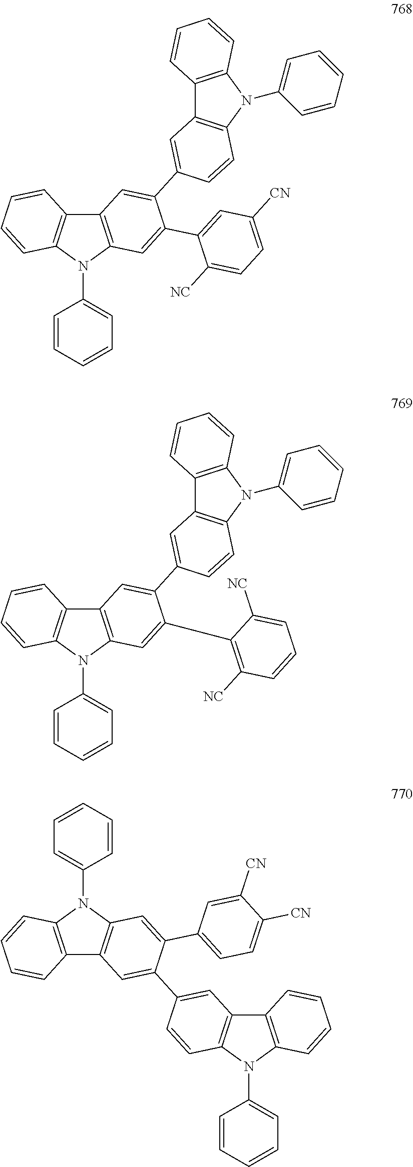

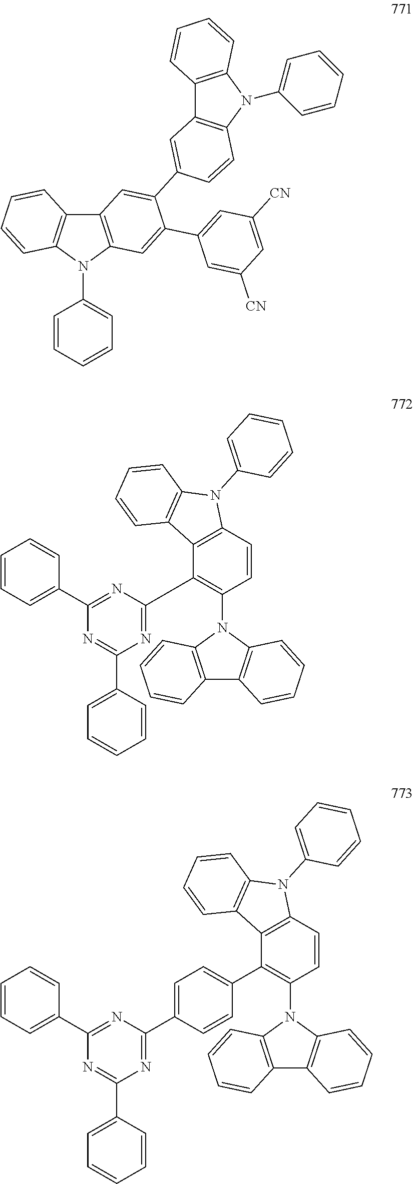 [SMILES: N#Cc1cc(C#N)cc(-c2cc3c(cc2-c2ccc4c(c2)c2ccccc2n4-c2ccccc2)c2ccccc2n3-c2ccccc2)c1 ; c1ccc(-c2nc(-c3ccccc3)nc(-c3c(-n4c5ccccc5c5ccccc54)ccc4c3c3ccccc3n4-c3ccccc3)n2)cc1 ; c1ccc(-c2nc(-c3ccccc3)nc(-c3ccc(-c4c(-n5c6ccccc6c6ccccc65)ccc5c4c4ccccc4n5-c4ccccc4)cc3)n2)cc1]

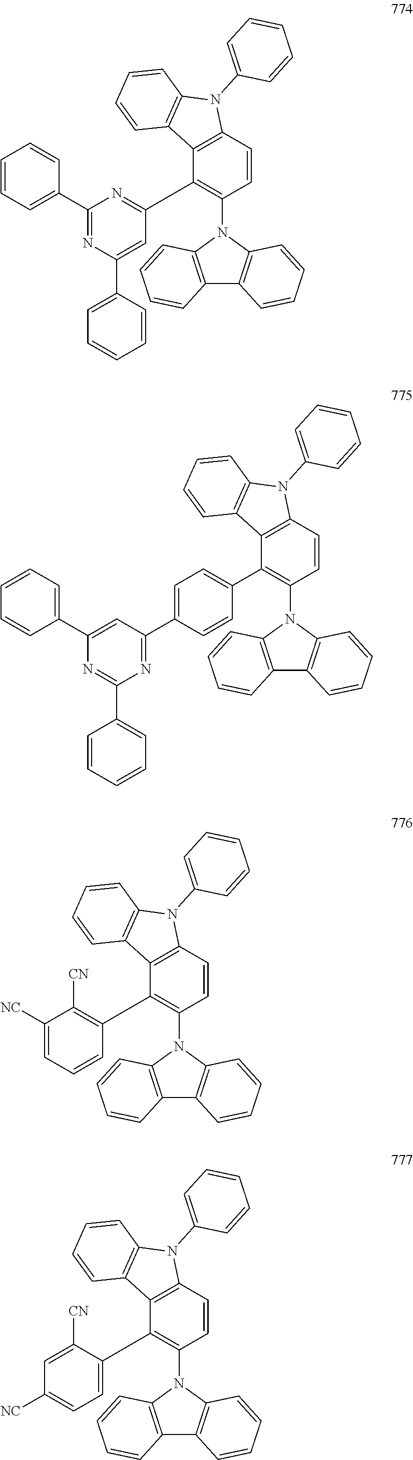 [SMILES: c1ccc(-c2cc(-c3c(-n4c5ccccc5c5ccccc54)ccc4c3c3ccccc3n4-c3ccccc3)nc(-c3ccccc3)n2)cc1 ; c1ccc(-c2cc(-c3ccc(-c4c(-n5c6ccccc6c6ccccc65)ccc5c4c4ccccc4n5-c4ccccc4)cc3)nc(-c3ccccc3)n2)cc1 ; N#Cc1cccc(-c2c(-n3c4ccccc4c4ccccc43)ccc3c2c2ccccc2n3-c2ccccc2)c1C#N ; N#Cc1ccc(-c2c(-n3c4ccccc4c4ccccc43)ccc3c2c2ccccc2n3-c2ccccc2)c(C#N)c1]

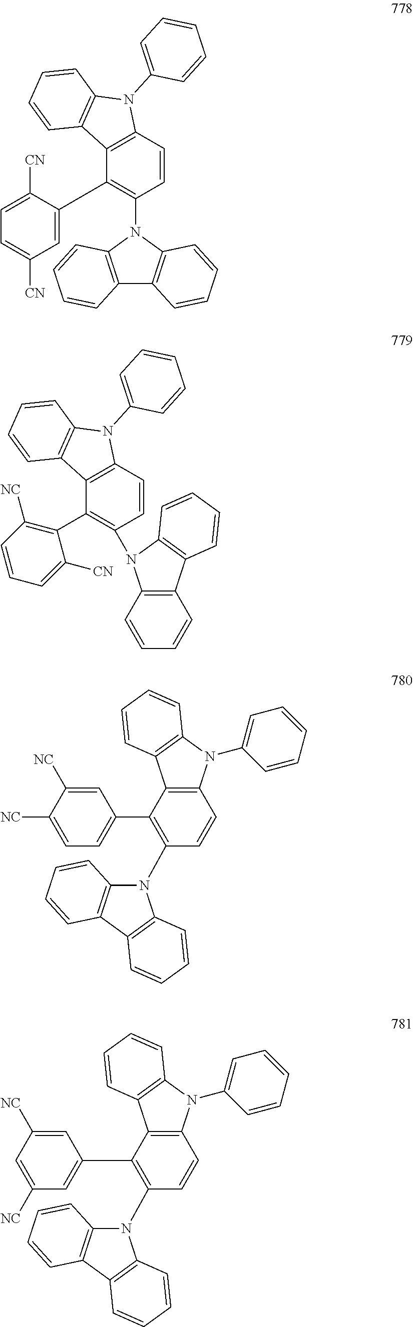

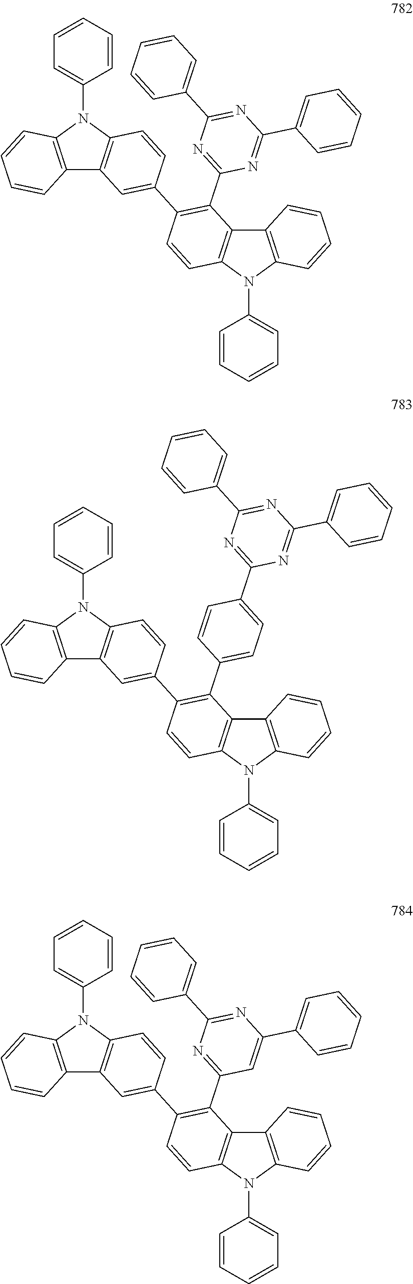 [SMILES: c1ccc(-c2nc(-c3ccccc3)nc(-c3c(-c4ccc5c(c4)c4ccccc4n5-c4ccccc4)ccc4c3c3ccccc3n4-c3ccccc3)n2)cc1 ; c1ccc(-c2nc(-c3ccccc3)nc(-c3ccc(-c4c(-c5ccc6c(c5)c5ccccc5n6-c5ccccc5)ccc5c4c4ccccc4n5-c4ccccc4)cc3)n2)cc1 ; c1ccc(-c2cc(-c3c(-c4ccc5c(c4)c4ccccc4n5-c4ccccc4)ccc4c3c3ccccc3n4-c3ccccc3)nc(-c3ccccc3)n2)cc1]

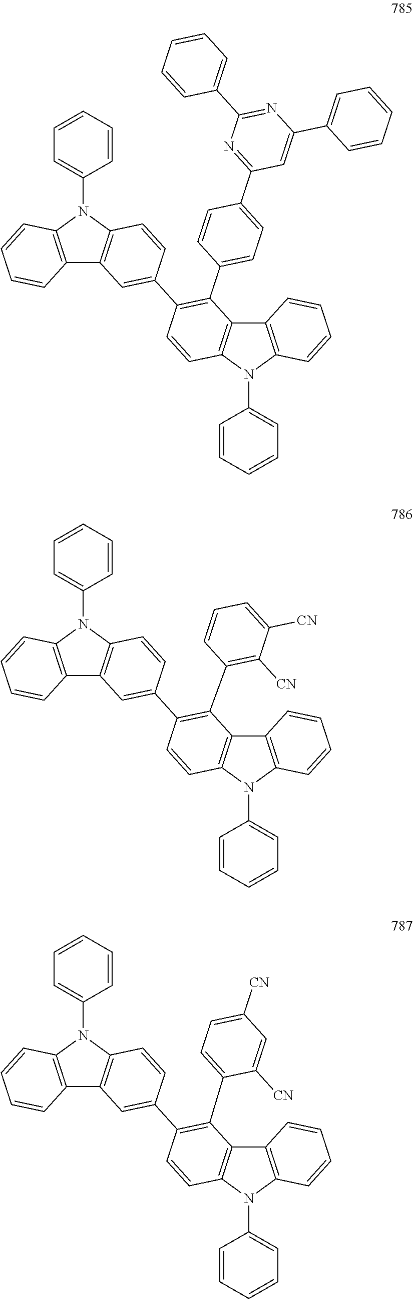 [SMILES: c1ccc(-c2cc(-c3ccc(-c4c(-c5ccc6c(c5)c5ccccc5n6-c5ccccc5)ccc5c4c4ccccc4n5-c4ccccc4)cc3)nc(-c3ccccc3)n2)cc1 ; N#Cc1cccc(-c2c(-c3ccc4c(c3)c3ccccc3n4-c3ccccc3)ccc3c2c2ccccc2n3-c2ccccc2)c1C#N ; N#Cc1ccc(-c2c(-c3ccc4c(c3)c3ccccc3n4-c3ccccc3)ccc3c2c2ccccc2n3-c2ccccc2)c(C#N)c1]

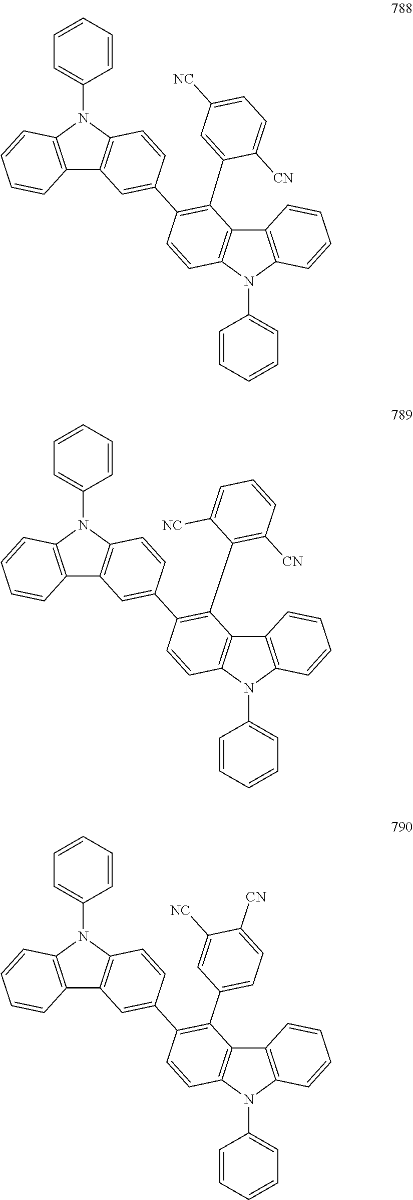 [SMILES: N#Cc1ccc(C#N)c(-c2c(-c3ccc4c(c3)c3ccccc3n4-c3ccccc3)ccc3c2c2ccccc2n3-c2ccccc2)c1 ; N#Cc1cccc(C#N)c1-c1c(-c2ccc3c(c2)c2ccccc2n3-c2ccccc2)ccc2c1c1ccccc1n2-c1ccccc1 ; N#Cc1ccc(-c2c(-c3ccc4c(c3)c3ccccc3n4-c3ccccc3)ccc3c2c2ccccc2n3-c2ccccc2)cc1C#N]

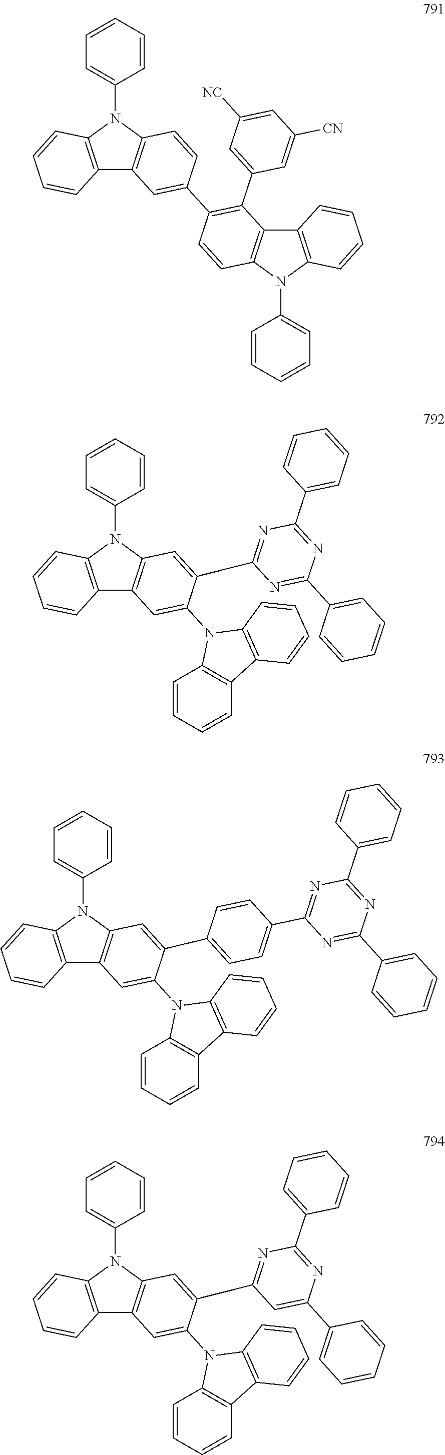 [SMILES: N#Cc1cc(C#N)cc(-c2c(-c3ccc4c(c3)c3ccccc3n4-c3ccccc3)ccc3c2c2ccccc2n3-c2ccccc2)c1 ; c1ccc(-c2nc(-c3ccccc3)nc(-c3cc4c(cc3-n3c5ccccc5c5ccccc53)c3ccccc3n4-c3ccccc3)n2)cc1 ; c1ccc(-c2nc(-c3ccccc3)nc(-c3ccc(-c4cc5c(cc4-n4c6ccccc6c6ccccc64)c4ccccc4n5-c4ccccc4)cc3)n2)cc1 ; c1ccc(-c2cc(-c3cc4c(cc3-n3c5ccccc5c5ccccc53)c3ccccc3n4-c3ccccc3)nc(-c3ccccc3)n2)cc1]

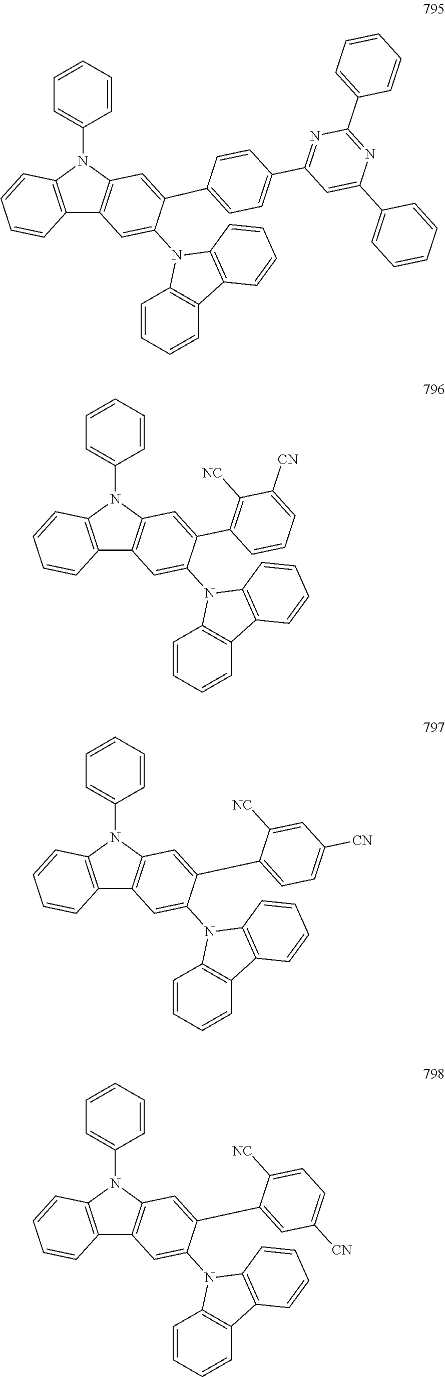 [SMILES: c1ccc(-c2cc(-c3ccc(-c4cc5c(cc4-n4c6ccccc6c6ccccc64)c4ccccc4n5-c4ccccc4)cc3)nc(-c3ccccc3)n2)cc1 ; N#Cc1cccc(-c2cc3c(cc2-n2c4ccccc4c4ccccc42)c2ccccc2n3-c2ccccc2)c1C#N ; N#Cc1ccc(-c2cc3c(cc2-n2c4ccccc4c4ccccc42)c2ccccc2n3-c2ccccc2)c(C#N)c1 ; N#Cc1ccc(C#N)c(-c2cc3c(cc2-n2c4ccccc4c4ccccc42)c2ccccc2n3-c2ccccc2)c1]

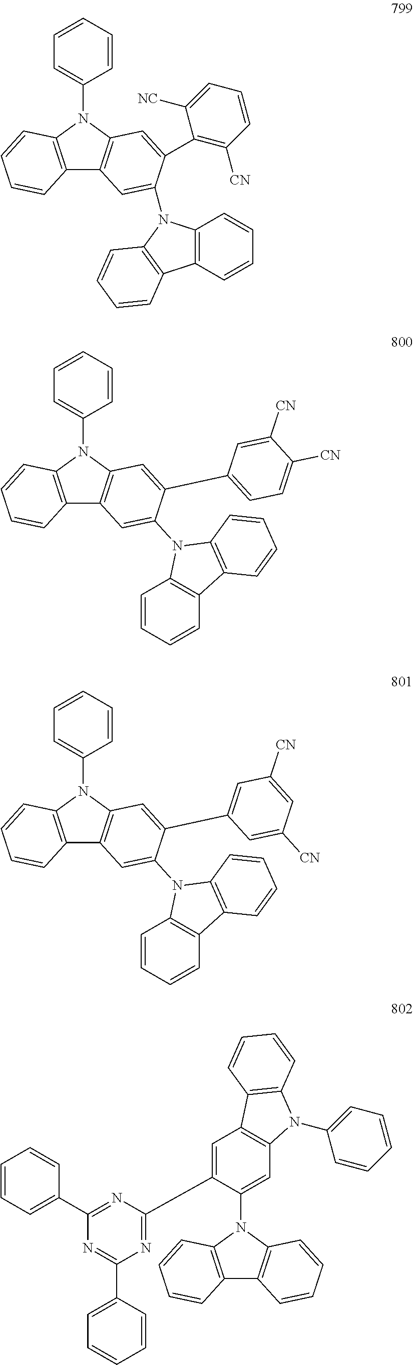 [SMILES: N#Cc1cccc(C#N)c1-c1cc2c(cc1-n1c3ccccc3c3ccccc31)c1ccccc1n2-c1ccccc1 ; N#Cc1ccc(-c2cc3c(cc2-n2c4ccccc4c4ccccc42)c2ccccc2n3-c2ccccc2)cc1C#N ; N#Cc1cc(C#N)cc(-c2cc3c(cc2-n2c4ccccc4c4ccccc42)c2ccccc2n3-c2ccccc2)c1 ; c1ccc(-c2nc(-c3ccccc3)nc(-c3cc4c5ccccc5n(-c5ccccc5)c4cc3-n3c4ccccc4c4ccccc43)n2)cc1]

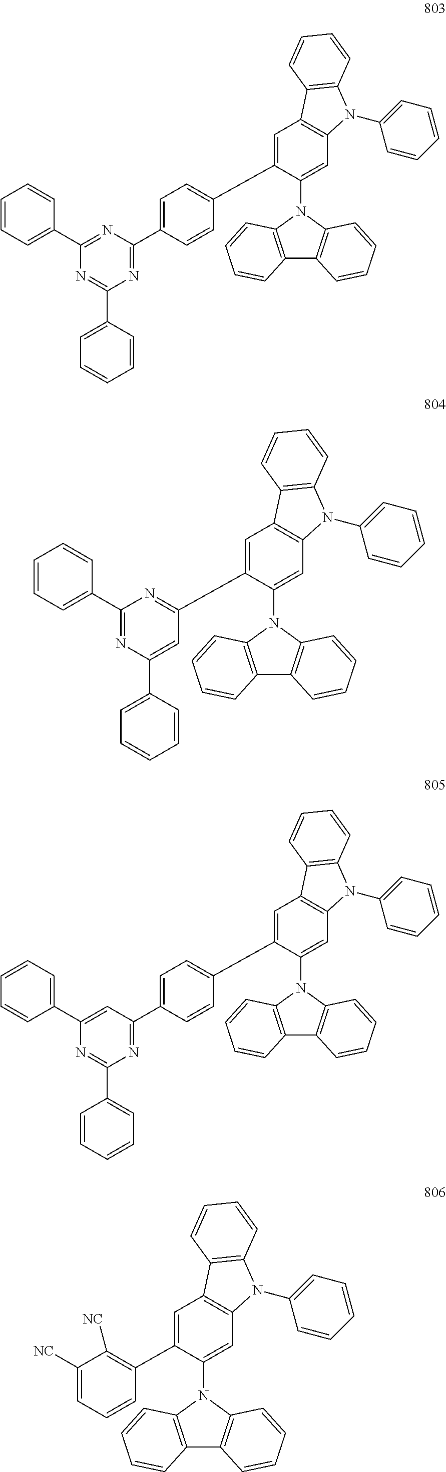 [SMILES: c1ccc(-c2nc(-c3ccccc3)nc(-c3ccc(-c4cc5c6ccccc6n(-c6ccccc6)c5cc4-n4c5ccccc5c5ccccc54)cc3)n2)cc1 ; c1ccc(-c2cc(-c3cc4c5ccccc5n(-c5ccccc5)c4cc3-n3c4ccccc4c4ccccc43)nc(-c3ccccc3)n2)cc1 ; c1ccc(-c2cc(-c3ccc(-c4cc5c6ccccc6n(-c6ccccc6)c5cc4-n4c5ccccc5c5ccccc54)cc3)nc(-c3ccccc3)n2)cc1 ; N#Cc1cccc(-c2cc3c4ccccc4n(-c4ccccc4)c3cc2-n2c3ccccc3c3ccccc32)c1C#N]

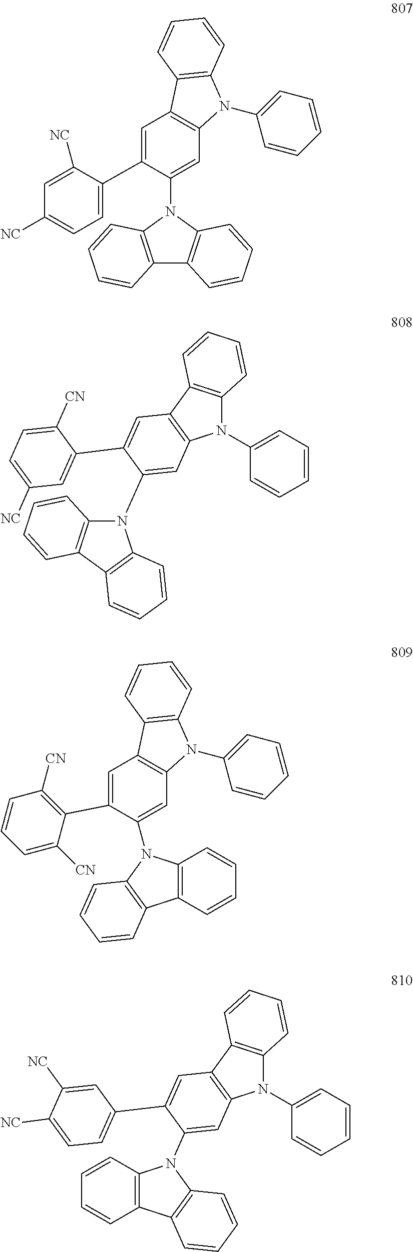

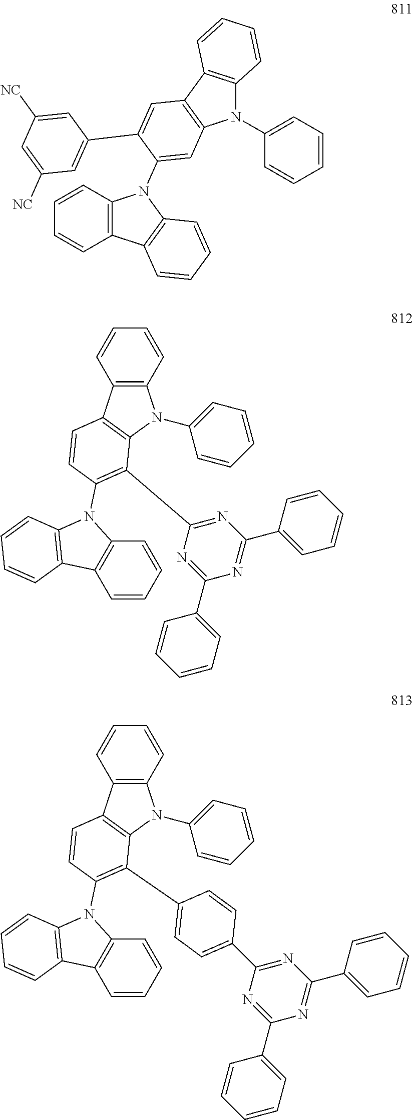 [SMILES: N#Cc1cc(C#N)cc(-c2cc3c4ccccc4n(-c4ccccc4)c3cc2-n2c3ccccc3c3ccccc32)c1 ; c1ccc(-c2nc(-c3ccccc3)nc(-c3c(-n4c5ccccc5c5ccccc54)ccc4c5ccccc5n(-c5ccccc5)c34)n2)cc1 ; c1ccc(-c2nc(-c3ccccc3)nc(-c3ccc(-c4c(-n5c6ccccc6c6ccccc65)ccc5c6ccccc6n(-c6ccccc6)c45)cc3)n2)cc1]

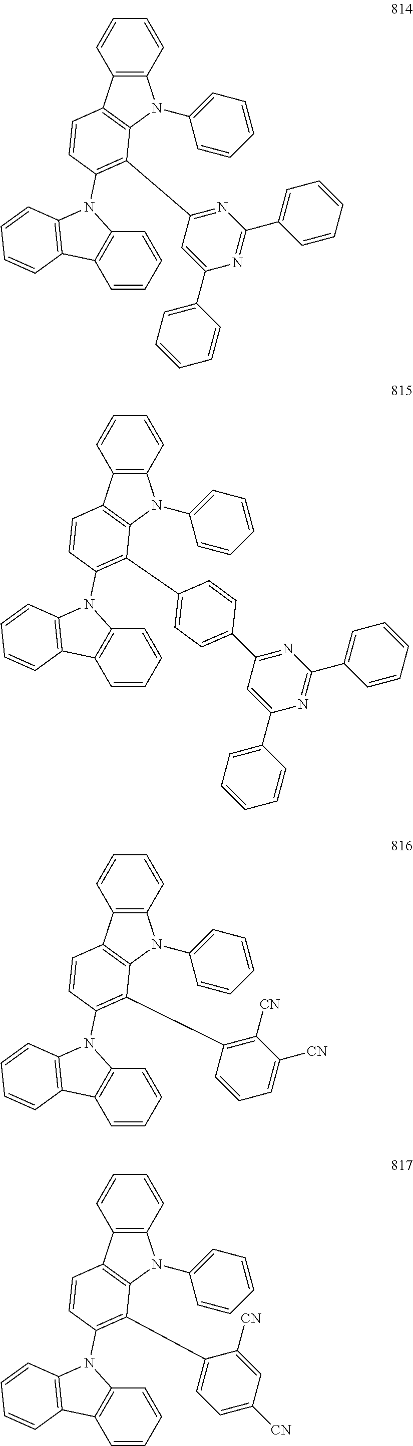 [SMILES: c1ccc(-c2cc(-c3c(-n4c5ccccc5c5ccccc54)ccc4c5ccccc5n(-c5ccccc5)c34)nc(-c3ccccc3)n2)cc1 ; c1ccc(-c2cc(-c3ccc(-c4c(-n5c6ccccc6c6ccccc65)ccc5c6ccccc6n(-c6ccccc6)c45)cc3)nc(-c3ccccc3)n2)cc1 ; N#Cc1cccc(-c2c(-n3c4ccccc4c4ccccc43)ccc3c4ccccc4n(-c4ccccc4)c23)c1C#N ; N#Cc1ccc(-c2c(-n3c4ccccc4c4ccccc43)ccc3c4ccccc4n(-c4ccccc4)c23)c(C#N)c1]

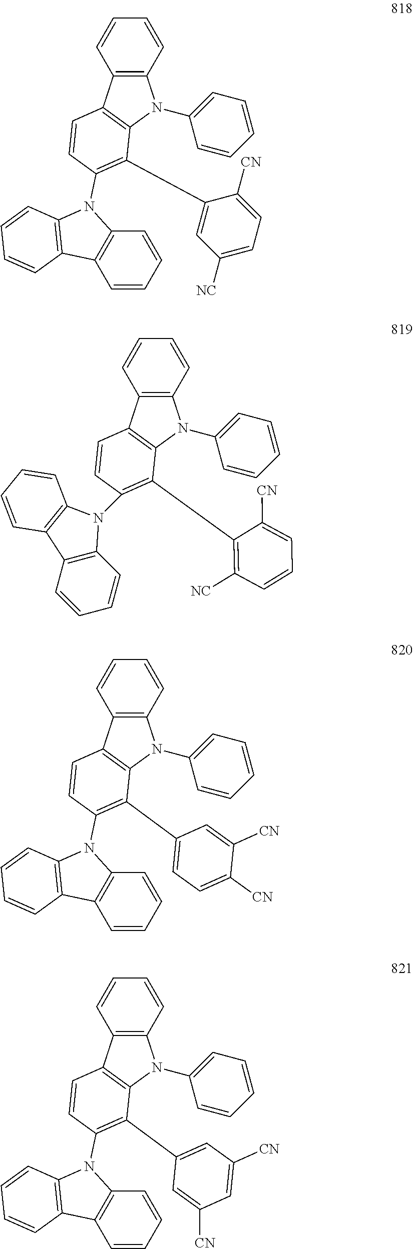 [SMILES: N#Cc1ccc(C#N)c(-c2c(-n3c4ccccc4c4ccccc43)ccc3c4ccccc4n(-c4ccccc4)c23)c1 ; N#Cc1cccc(C#N)c1-c1c(-n2c3ccccc3c3ccccc32)ccc2c3ccccc3n(-c3ccccc3)c12 ; N#Cc1ccc(-c2c(-n3c4ccccc4c4ccccc43)ccc3c4ccccc4n(-c4ccccc4)c23)cc1C#N ; N#Cc1cc(C#N)cc(-c2c(-n3c4ccccc4c4ccccc43)ccc3c4ccccc4n(-c4ccccc4)c23)c1]

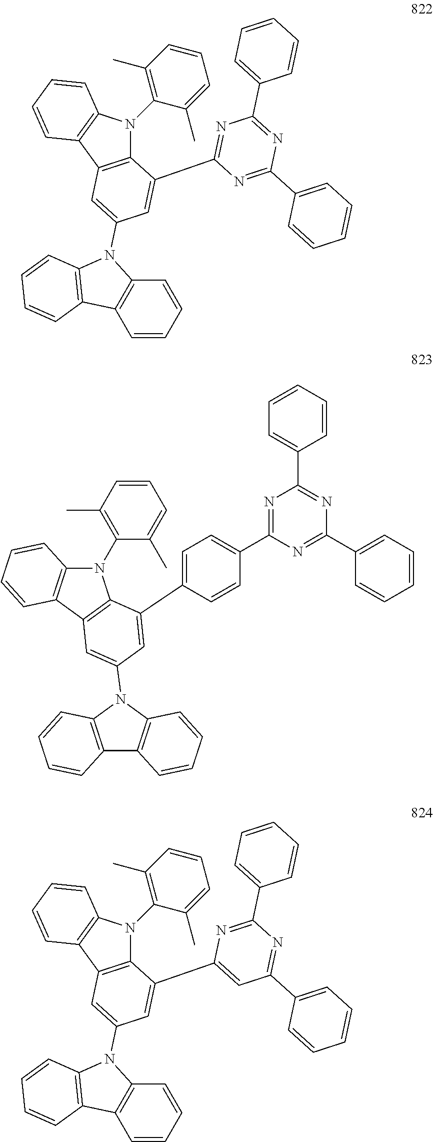 [SMILES: Cc1cccc(C)c1-n1c2ccccc2c2cc(-n3c4ccccc4c4ccccc43)cc(-c3nc(-c4ccccc4)nc(-c4ccccc4)n3)c21 ; Cc1cccc(C)c1-n1c2ccccc2c2cc(-n3c4ccccc4c4ccccc43)cc(-c3ccc(-c4nc(-c5ccccc5)nc(-c5ccccc5)n4)cc3)c21 ; Cc1cccc(C)c1-n1c2ccccc2c2cc(-n3c4ccccc4c4ccccc43)cc(-c3cc(-c4ccccc4)nc(-c4ccccc4)n3)c21]

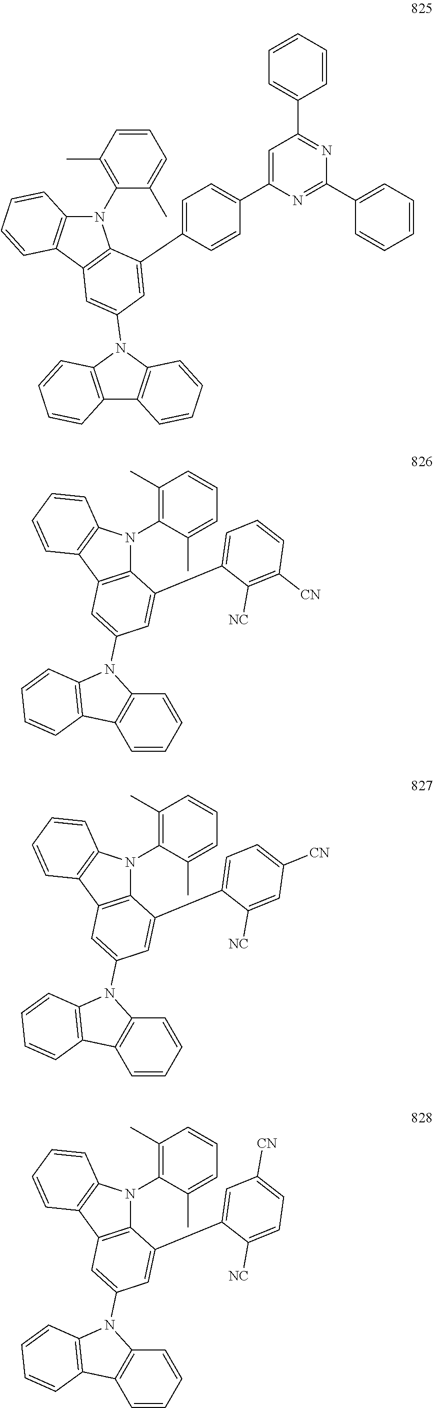

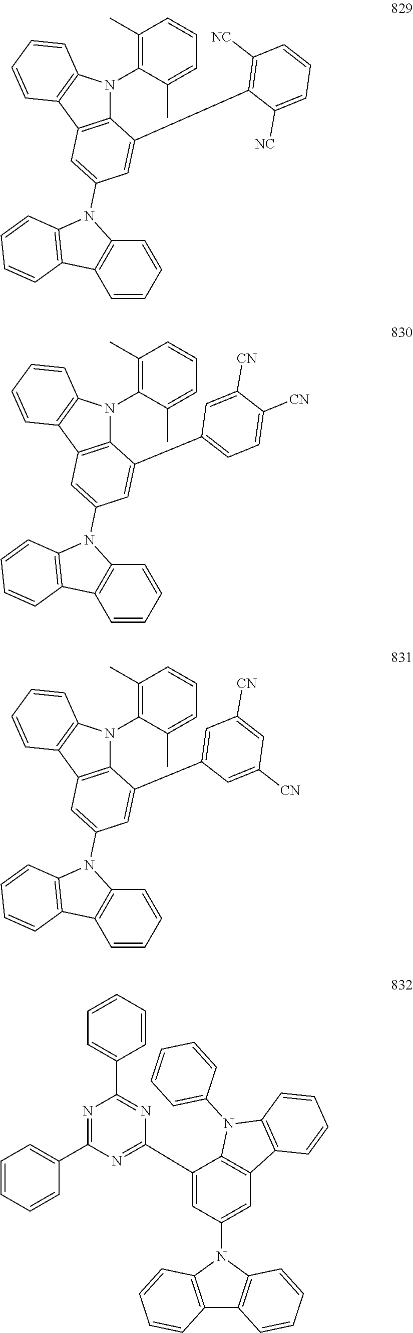

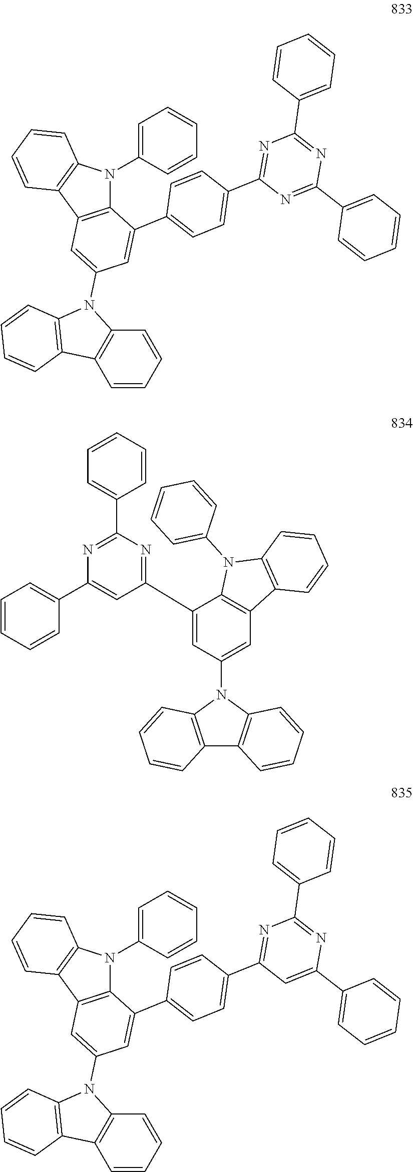

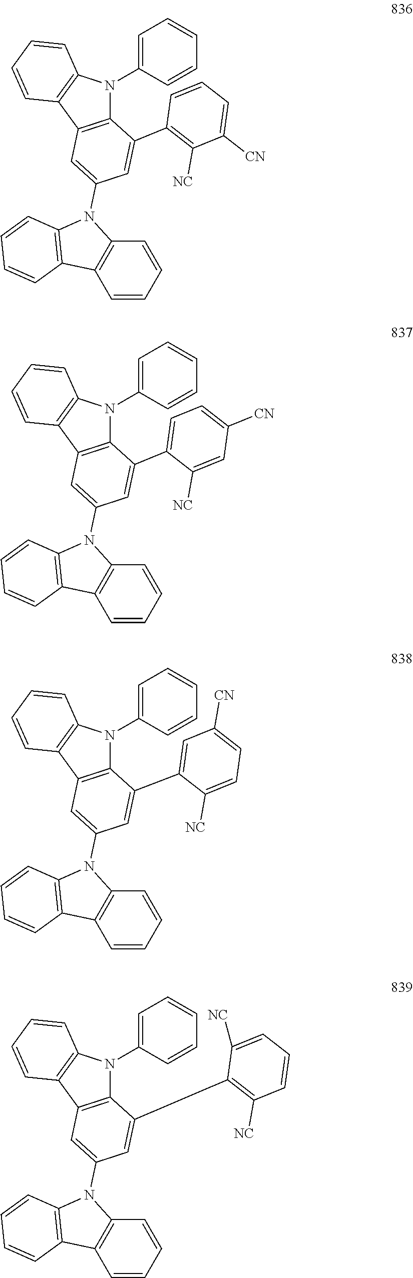 [SMILES: N#Cc1cccc(-c2cc(-n3c4ccccc4c4ccccc43)cc3c4ccccc4n(-c4ccccc4)c23)c1C#N ; N#Cc1ccc(-c2cc(-n3c4ccccc4c4ccccc43)cc3c4ccccc4n(-c4ccccc4)c23)c(C#N)c1 ; N#Cc1ccc(C#N)c(-c2cc(-n3c4ccccc4c4ccccc43)cc3c4ccccc4n(-c4ccccc4)c23)c1 ; N#Cc1cccc(C#N)c1-c1cc(-n2c3ccccc3c3ccccc32)cc2c3ccccc3n(-c3ccccc3)c12]

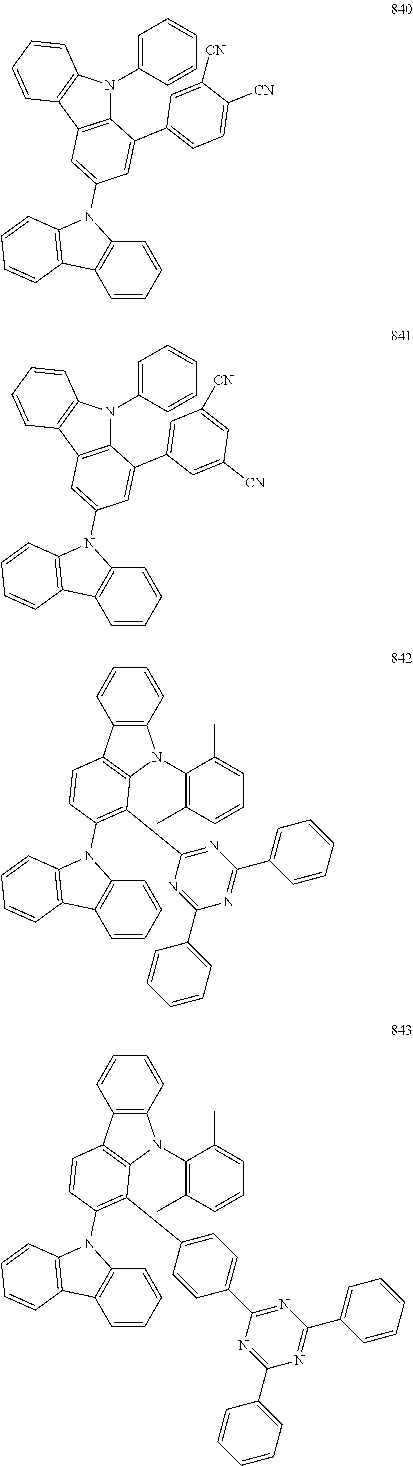

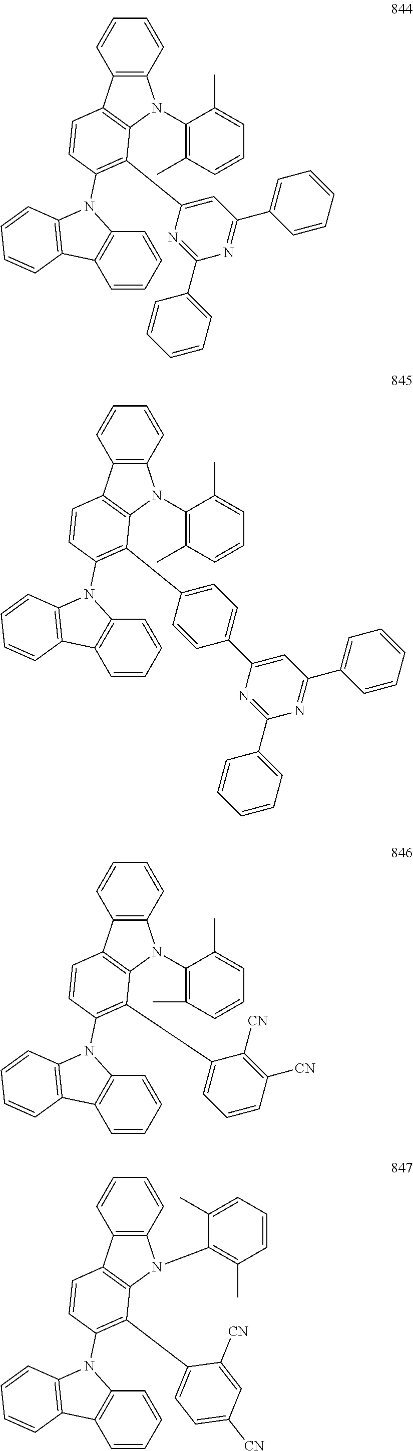

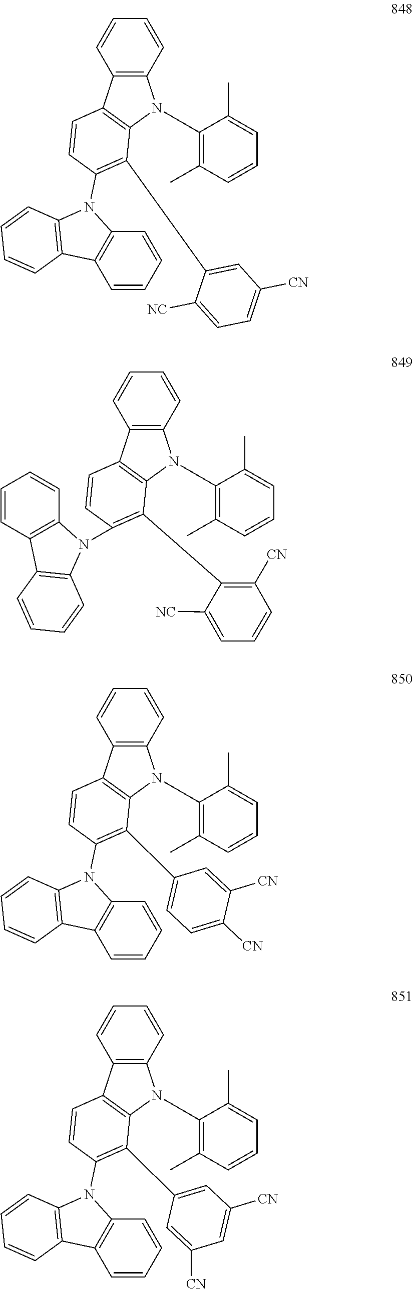 [SMILES: Cc1cccc(C)c1-n1c2ccccc2c2ccc(-n3c4ccccc4c4ccccc43)c(-c3cc(C#N)ccc3C#N)c21 ; Cc1cccc(C)c1-n1c2ccccc2c2ccc(-n3c4ccccc4c4ccccc43)c(-c3c(C#N)cccc3C#N)c21 ; Cc1cccc(C)c1-n1c2ccccc2c2ccc(-n3c4ccccc4c4ccccc43)c(-c3ccc(C#N)c(C#N)c3)c21 ; Cc1cccc(C)c1-n1c2ccccc2c2ccc(-n3c4ccccc4c4ccccc43)c(-c3cc(C#N)cc(C#N)c3)c21]

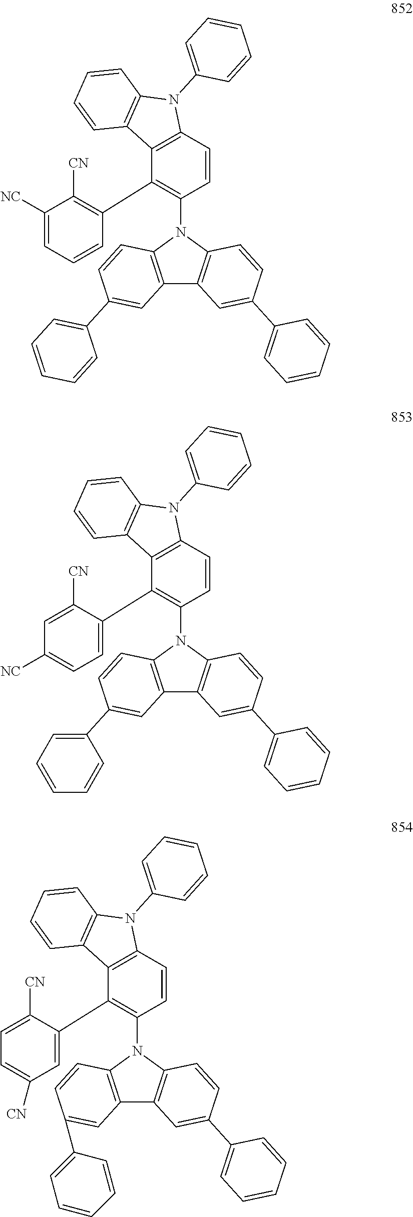

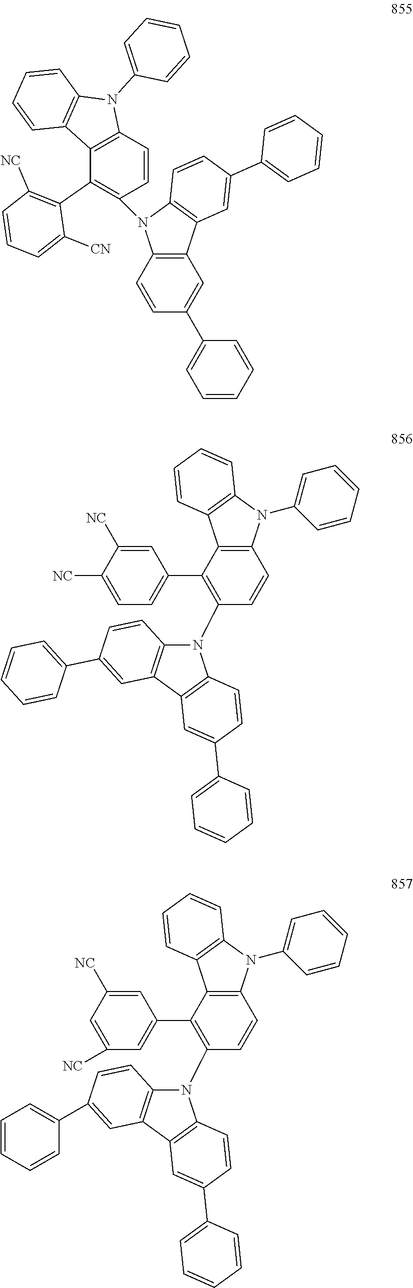 [SMILES: N#Cc1cccc(C#N)c1-c1c(-n2c3ccc(-c4ccccc4)cc3c3cc(-c4ccccc4)ccc32)ccc2c1c1ccccc1n2-c1ccccc1 ; N#Cc1ccc(-c2c(-n3c4ccc(-c5ccccc5)cc4c4cc(-c5ccccc5)ccc43)ccc3c2c2ccccc2n3-c2ccccc2)cc1C#N ; N#Cc1cc(C#N)cc(-c2c(-n3c4ccc(-c5ccccc5)cc4c4cc(-c5ccccc5)ccc43)ccc3c2c2ccccc2n3-c2ccccc2)c1]

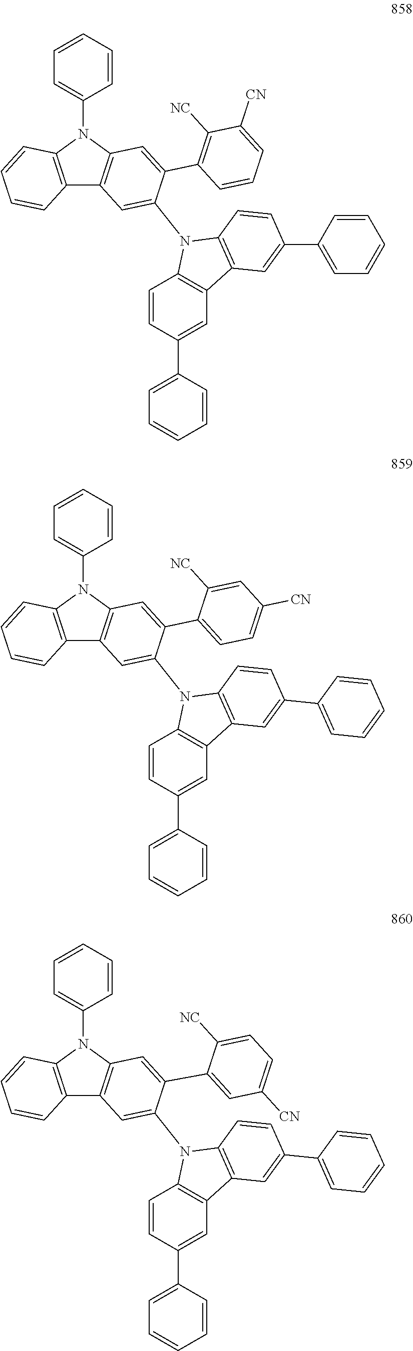 [SMILES: N#Cc1cccc(-c2cc3c(cc2-n2c4ccc(-c5ccccc5)cc4c4cc(-c5ccccc5)ccc42)c2ccccc2n3-c2ccccc2)c1C#N ; N#Cc1ccc(-c2cc3c(cc2-n2c4ccc(-c5ccccc5)cc4c4cc(-c5ccccc5)ccc42)c2ccccc2n3-c2ccccc2)c(C#N)c1 ; N#Cc1ccc(C#N)c(-c2cc3c(cc2-n2c4ccc(-c5ccccc5)cc4c4cc(-c5ccccc5)ccc42)c2ccccc2n3-c2ccccc2)c1]

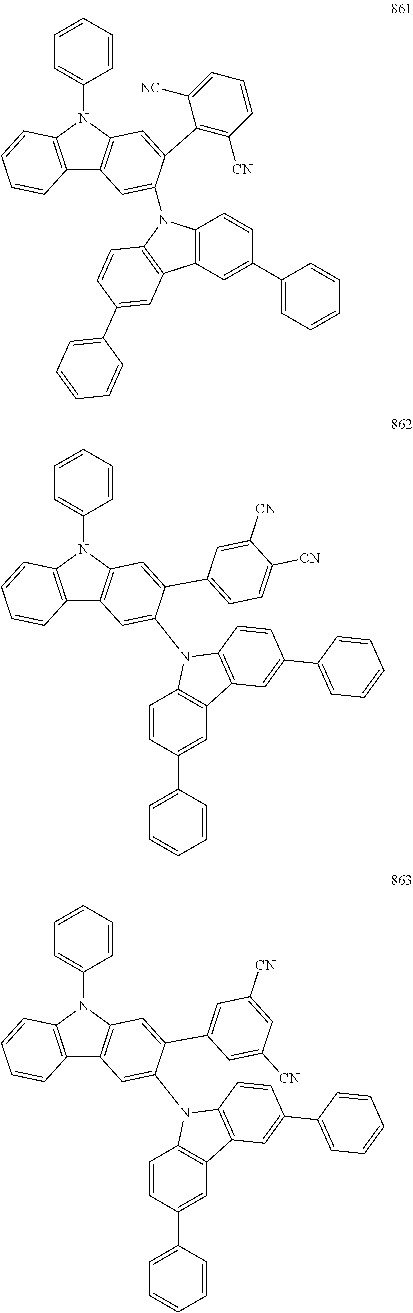 [SMILES: N#Cc1cccc(C#N)c1-c1cc2c(cc1-n1c3ccc(-c4ccccc4)cc3c3cc(-c4ccccc4)ccc31)c1ccccc1n2-c1ccccc1 ; N#Cc1ccc(-c2cc3c(cc2-n2c4ccc(-c5ccccc5)cc4c4cc(-c5ccccc5)ccc42)c2ccccc2n3-c2ccccc2)cc1C#N ; N#Cc1cc(C#N)cc(-c2cc3c(cc2-n2c4ccc(-c5ccccc5)cc4c4cc(-c5ccccc5)ccc42)c2ccccc2n3-c2ccccc2)c1]

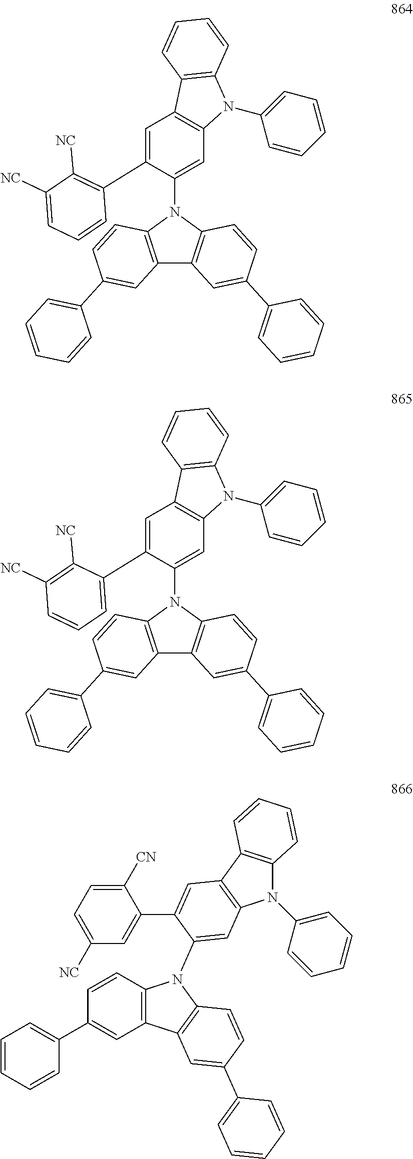 [SMILES: N#Cc1cccc(-c2cc3c4ccccc4n(-c4ccccc4)c3cc2-n2c3ccc(-c4ccccc4)cc3c3cc(-c4ccccc4)ccc32)c1C#N ; N#Cc1cccc(-c2cc3c4ccccc4n(-c4ccccc4)c3cc2-n2c3ccc(-c4ccccc4)cc3c3cc(-c4ccccc4)ccc32)c1C#N ; N#Cc1ccc(C#N)c(-c2cc3c4ccccc4n(-c4ccccc4)c3cc2-n2c3ccc(-c4ccccc4)cc3c3cc(-c4ccccc4)ccc32)c1]

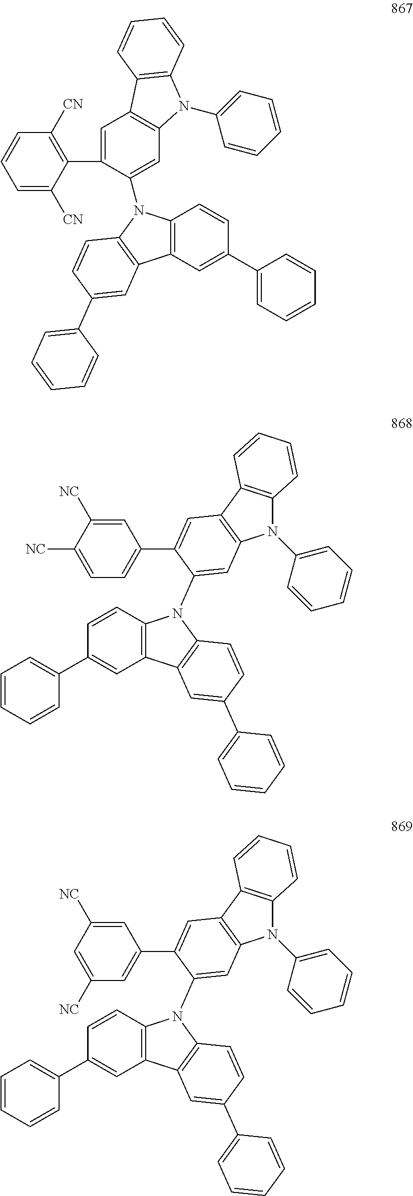

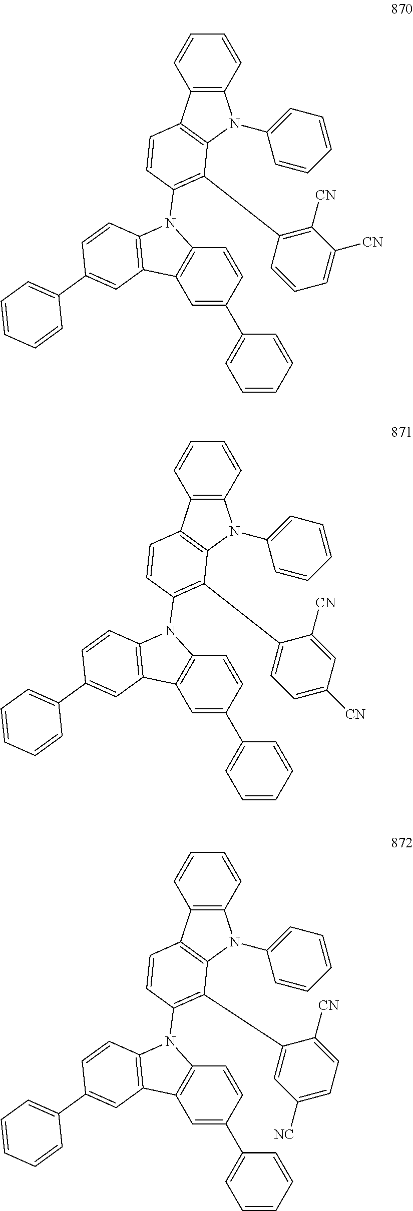 [SMILES: N#Cc1cccc(-c2c(-n3c4ccc(-c5ccccc5)cc4c4cc(-c5ccccc5)ccc43)ccc3c4ccccc4n(-c4ccccc4)c23)c1C#N ; N#Cc1ccc(-c2c(-n3c4ccc(-c5ccccc5)cc4c4cc(-c5ccccc5)ccc43)ccc3c4ccccc4n(-c4ccccc4)c23)c(C#N)c1 ; N#Cc1ccc(C#N)c(-c2c(-n3c4ccc(-c5ccccc5)cc4c4cc(-c5ccccc5)ccc43)ccc3c4ccccc4n(-c4ccccc4)c23)c1]

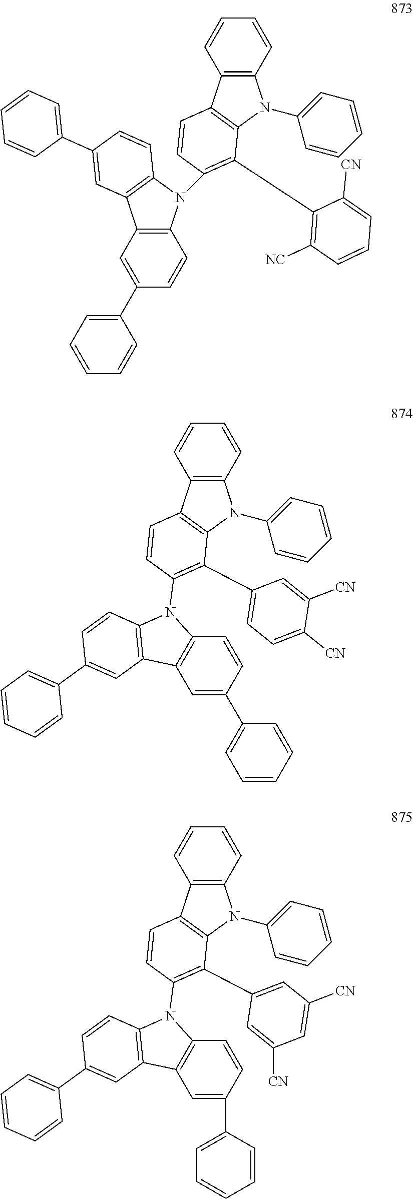 [SMILES: N#Cc1cccc(C#N)c1-c1c(-n2c3ccc(-c4ccccc4)cc3c3cc(-c4ccccc4)ccc32)ccc2c3ccccc3n(-c3ccccc3)c12 ; N#Cc1ccc(-c2c(-n3c4ccc(-c5ccccc5)cc4c4cc(-c5ccccc5)ccc43)ccc3c4ccccc4n(-c4ccccc4)c23)cc1C#N ; N#Cc1cc(C#N)cc(-c2c(-n3c4ccc(-c5ccccc5)cc4c4cc(-c5ccccc5)ccc43)ccc3c4ccccc4n(-c4ccccc4)c23)c1]

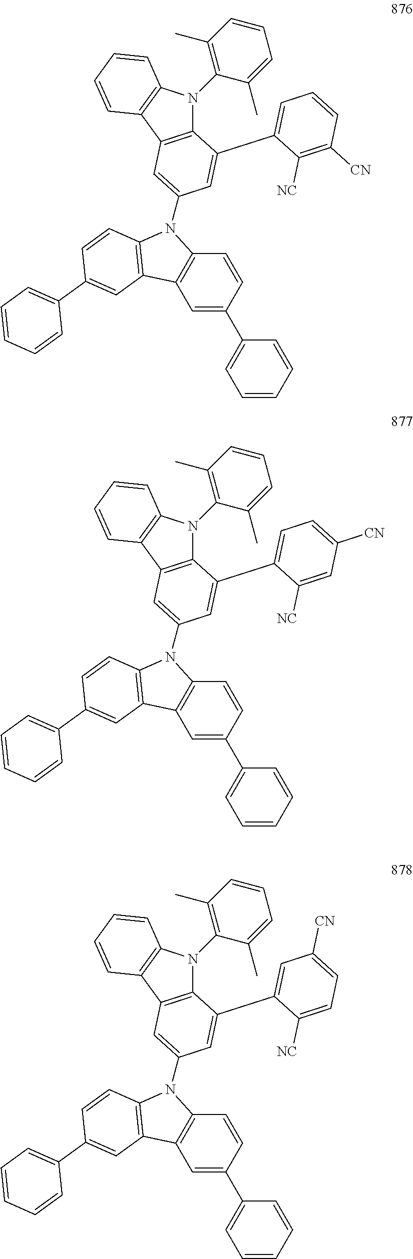 [SMILES: Cc1cccc(C)c1-n1c2ccccc2c2cc(-n3c4ccc(-c5ccccc5)cc4c4cc(-c5ccccc5)ccc43)cc(-c3cccc(C#N)c3C#N)c21 ; Cc1cccc(C)c1-n1c2ccccc2c2cc(-n3c4ccc(-c5ccccc5)cc4c4cc(-c5ccccc5)ccc43)cc(-c3ccc(C#N)cc3C#N)c21 ; Cc1cccc(C)c1-n1c2ccccc2c2cc(-n3c4ccc(-c5ccccc5)cc4c4cc(-c5ccccc5)ccc43)cc(-c3cc(C#N)ccc3C#N)c21]

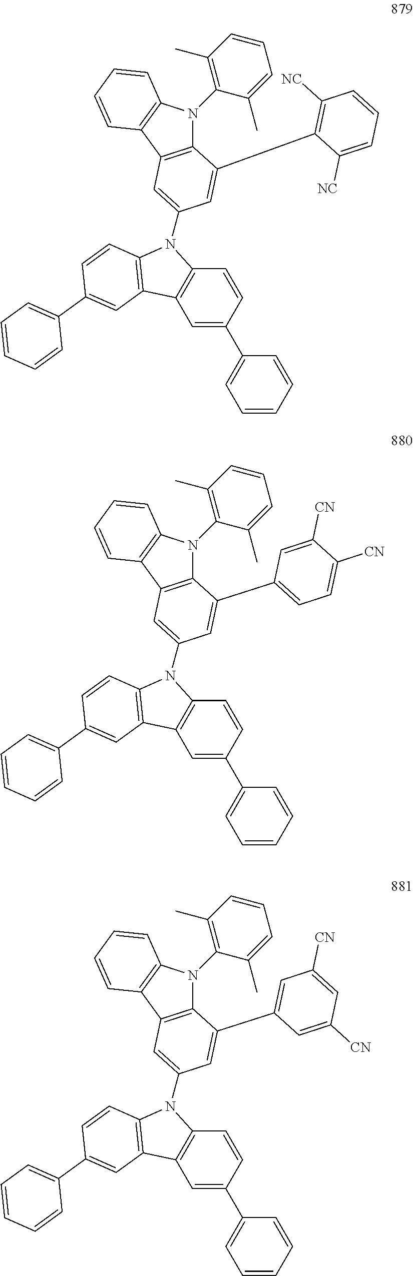 [SMILES: Cc1cccc(C)c1-n1c2ccccc2c2cc(-n3c4ccc(-c5ccccc5)cc4c4cc(-c5ccccc5)ccc43)cc(-c3c(C#N)cccc3C#N)c21 ; Cc1cccc(C)c1-n1c2ccccc2c2cc(-n3c4ccc(-c5ccccc5)cc4c4cc(-c5ccccc5)ccc43)cc(-c3ccc(C#N)c(C#N)c3)c21 ; Cc1cccc(C)c1-n1c2ccccc2c2cc(-n3c4ccc(-c5ccccc5)cc4c4cc(-c5ccccc5)ccc43)cc(-c3cc(C#N)cc(C#N)c3)c21]

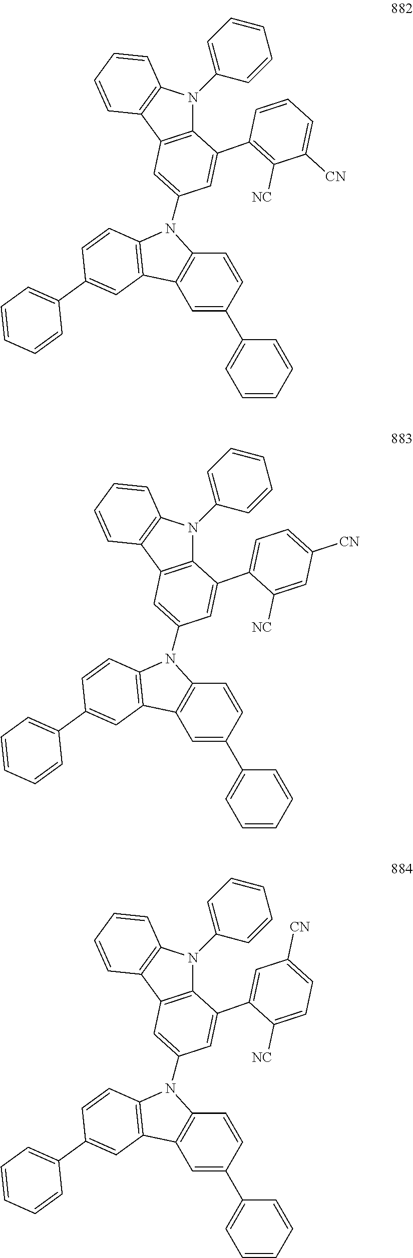 [SMILES: N#Cc1cccc(-c2cc(-n3c4ccc(-c5ccccc5)cc4c4cc(-c5ccccc5)ccc43)cc3c4ccccc4n(-c4ccccc4)c23)c1C#N ; N#Cc1ccc(-c2cc(-n3c4ccc(-c5ccccc5)cc4c4cc(-c5ccccc5)ccc43)cc3c4ccccc4n(-c4ccccc4)c23)c(C#N)c1 ; N#Cc1ccc(C#N)c(-c2cc(-n3c4ccc(-c5ccccc5)cc4c4cc(-c5ccccc5)ccc43)cc3c4ccccc4n(-c4ccccc4)c23)c1]

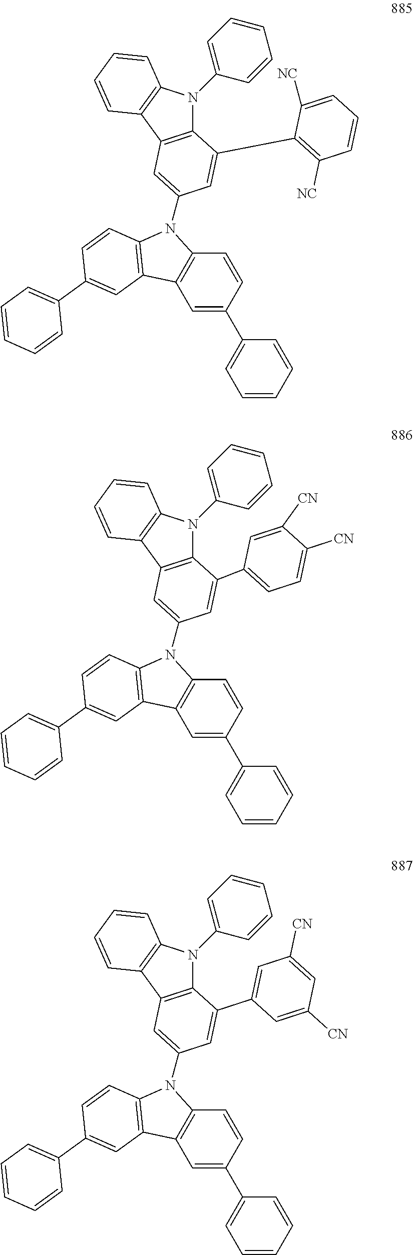 [SMILES: N#Cc1cccc(C#N)c1-c1cc(-n2c3ccc(-c4ccccc4)cc3c3cc(-c4ccccc4)ccc32)cc2c3ccccc3n(-c3ccccc3)c12 ; N#Cc1ccc(-c2cc(-n3c4ccc(-c5ccccc5)cc4c4cc(-c5ccccc5)ccc43)cc3c4ccccc4n(-c4ccccc4)c23)cc1C#N ; N#Cc1cc(C#N)cc(-c2cc(-n3c4ccc(-c5ccccc5)cc4c4cc(-c5ccccc5)ccc43)cc3c4ccccc4n(-c4ccccc4)c23)c1]

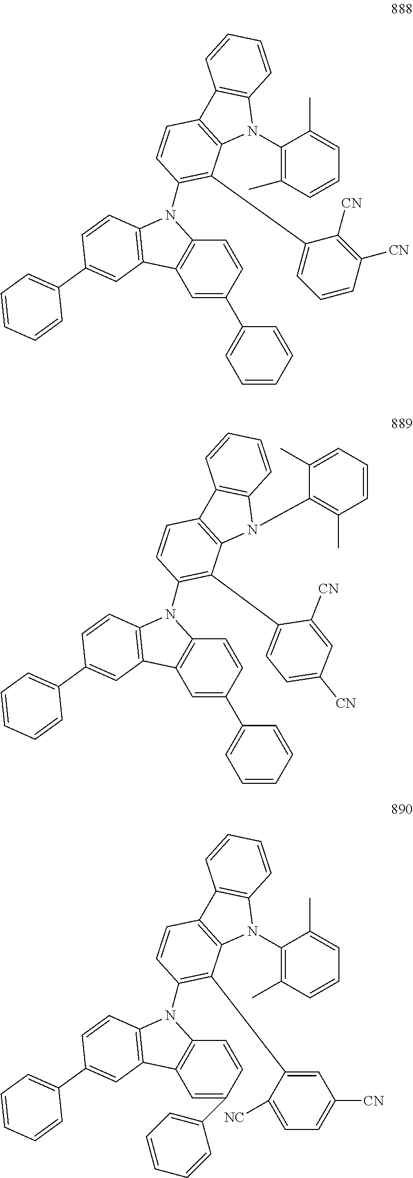 [SMILES: Cc1cccc(C)c1-n1c2ccccc2c2ccc(-n3c4ccc(-c5ccccc5)cc4c4cc(-c5ccccc5)ccc43)c(-c3cccc(C#N)c3C#N)c21 ; Cc1cccc(C)c1-n1c2ccccc2c2ccc(-n3c4ccc(-c5ccccc5)cc4c4cc(-c5ccccc5)ccc43)c(-c3ccc(C#N)cc3C#N)c21 ; Cc1cccc(C)c1-n1c2ccccc2c2ccc(-n3c4ccc(-c5ccccc5)cc4c4cc(-c5ccccc5)ccc43)c(-c3cc(C#N)ccc3C#N)c21]

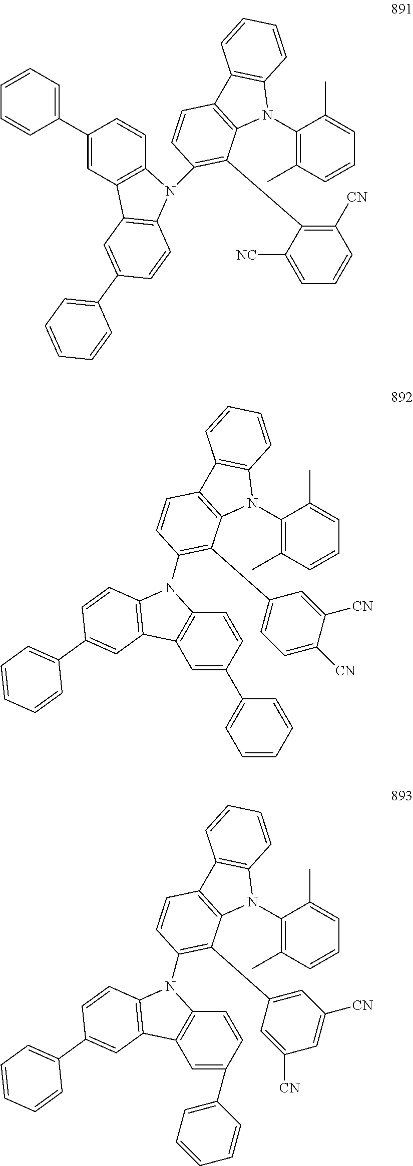 [SMILES: Cc1cccc(C)c1-n1c2ccccc2c2ccc(-n3c4ccc(-c5ccccc5)cc4c4cc(-c5ccccc5)ccc43)c(-c3c(C#N)cccc3C#N)c21 ; Cc1cccc(C)c1-n1c2ccccc2c2ccc(-n3c4ccc(-c5ccccc5)cc4c4cc(-c5ccccc5)ccc43)c(-c3ccc(C#N)c(C#N)c3)c21 ; Cc1cccc(C)c1-n1c2ccccc2c2ccc(-n3c4ccc(-c5ccccc5)cc4c4cc(-c5ccccc5)ccc43)c(-c3cc(C#N)cc(C#N)c3)c21]

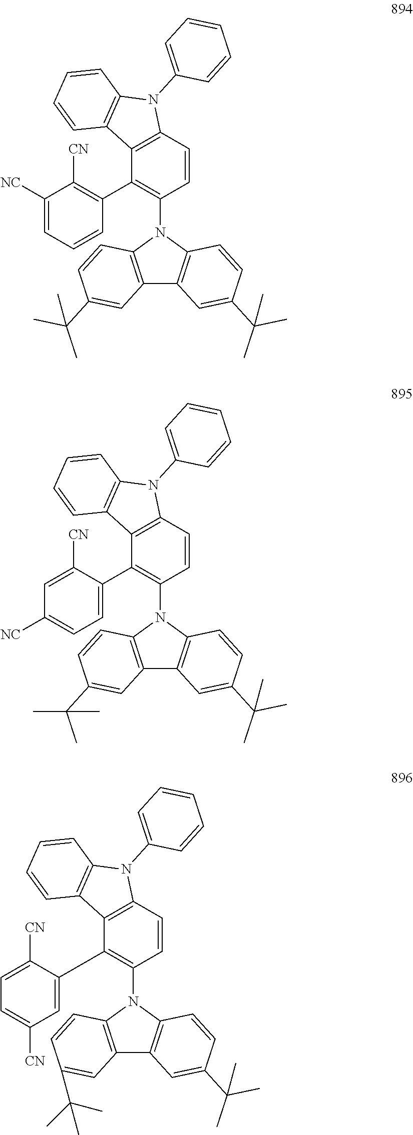 [SMILES: CC(C)(C)c1ccc2c(c1)c1cc(C(C)(C)C)ccc1n2-c1ccc2c(c1-c1cccc(C#N)c1C#N)c1ccccc1n2-c1ccccc1 ; CC(C)(C)c1ccc2c(c1)c1cc(C(C)(C)C)ccc1n2-c1ccc2c(c1-c1ccc(C#N)cc1C#N)c1ccccc1n2-c1ccccc1 ; CC(C)(C)c1ccc2c(c1)c1cc(C(C)(C)C)ccc1n2-c1ccc2c(c1-c1cc(C#N)ccc1C#N)c1ccccc1n2-c1ccccc1]

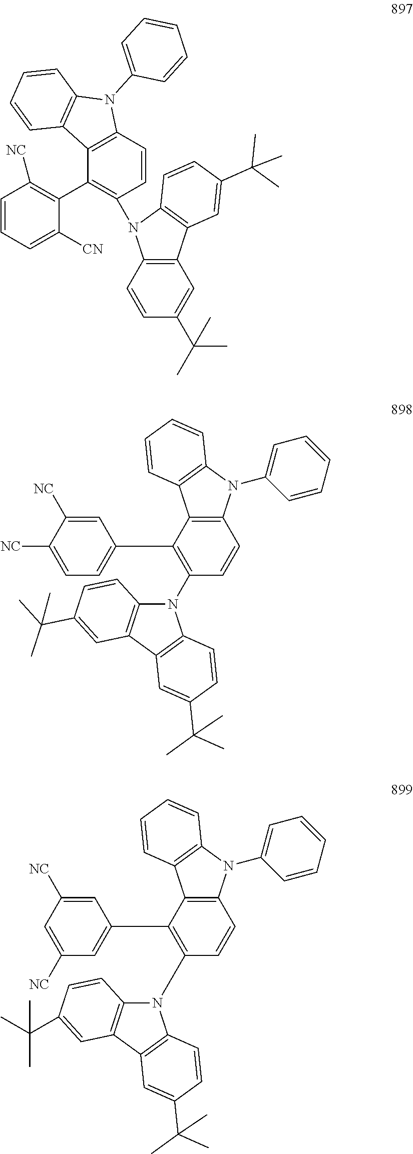

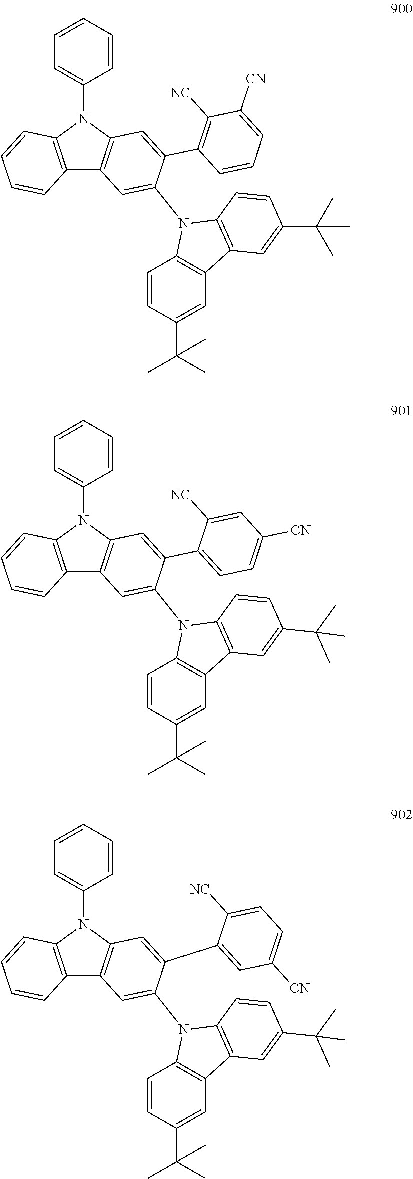 [SMILES: CC(C)(C)c1ccc2c(c1)c1cc(C(C)(C)C)ccc1n2-c1cc2c3ccccc3n(-c3ccccc3)c2cc1-c1cccc(C#N)c1C#N ; CC(C)(C)c1ccc2c(c1)c1cc(C(C)(C)C)ccc1n2-c1cc2c3ccccc3n(-c3ccccc3)c2cc1-c1ccc(C#N)cc1C#N ; CC(C)(C)c1ccc2c(c1)c1cc(C(C)(C)C)ccc1n2-c1cc2c3ccccc3n(-c3ccccc3)c2cc1-c1cc(C#N)ccc1C#N]

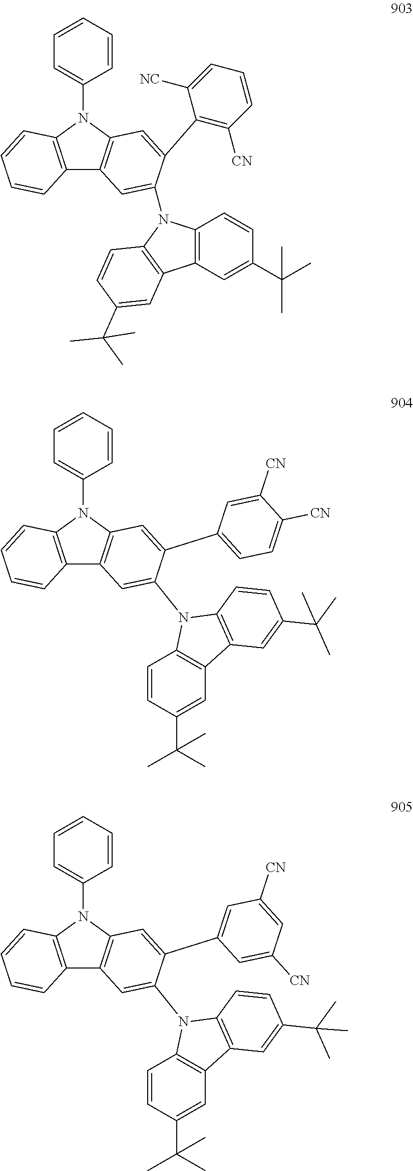 [SMILES: CC(C)(C)c1ccc2c(c1)c1cc(C(C)(C)C)ccc1n2-c1cc2c3ccccc3n(-c3ccccc3)c2cc1-c1c(C#N)cccc1C#N ; CC(C)(C)c1ccc2c(c1)c1cc(C(C)(C)C)ccc1n2-c1cc2c3ccccc3n(-c3ccccc3)c2cc1-c1ccc(C#N)c(C#N)c1 ; CC(C)(C)c1ccc2c(c1)c1cc(C(C)(C)C)ccc1n2-c1cc2c3ccccc3n(-c3ccccc3)c2cc1-c1cc(C#N)cc(C#N)c1]

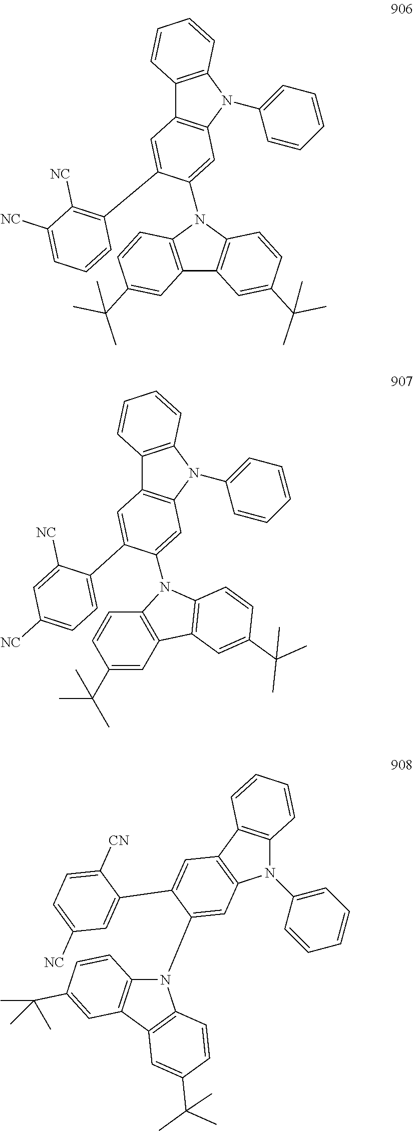

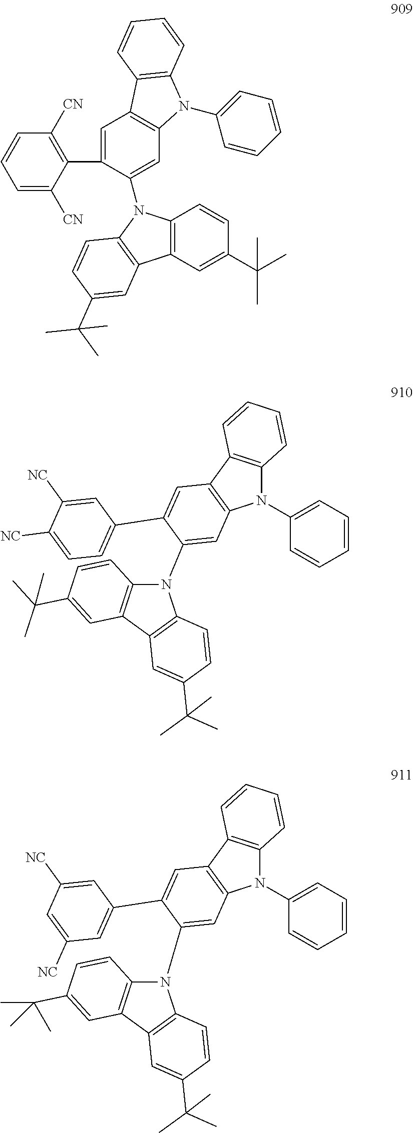

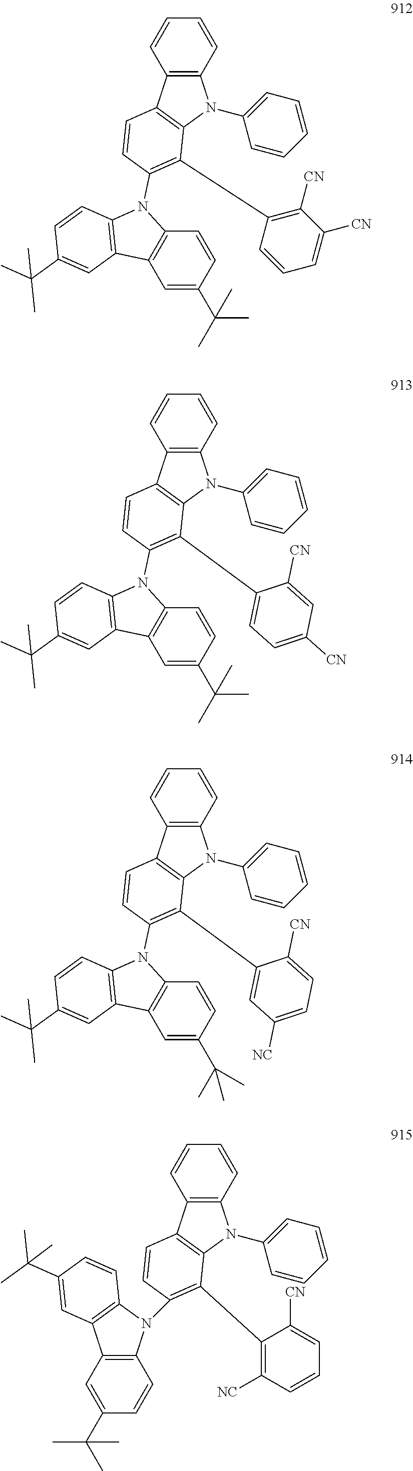

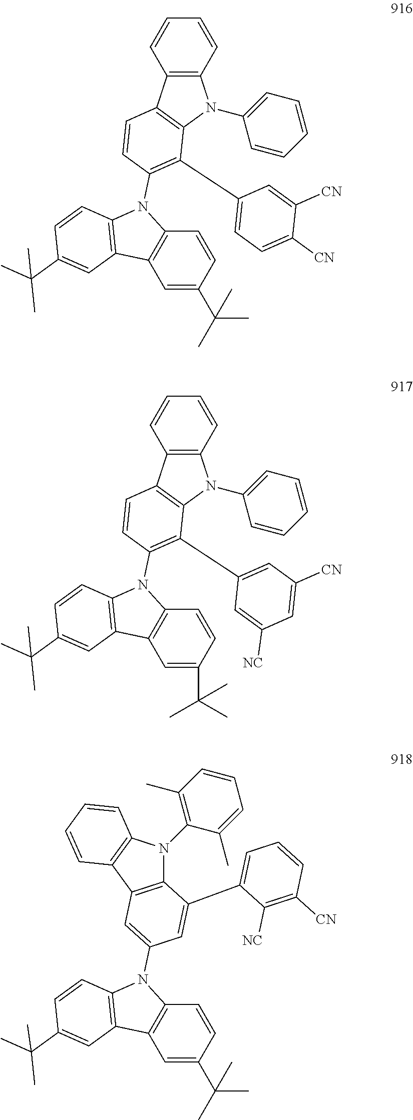 [SMILES: CC(C)(C)c1ccc2c(c1)c1cc(C(C)(C)C)ccc1n2-c1ccc2c3ccccc3n(-c3ccccc3)c2c1-c1ccc(C#N)c(C#N)c1 ; CC(C)(C)c1ccc2c(c1)c1cc(C(C)(C)C)ccc1n2-c1ccc2c3ccccc3n(-c3ccccc3)c2c1-c1cc(C#N)cc(C#N)c1 ; Cc1cccc(C)c1-n1c2ccccc2c2cc(-n3c4ccc(C(C)(C)C)cc4c4cc(C(C)(C)C)ccc43)cc(-c3cccc(C#N)c3C#N)c21]

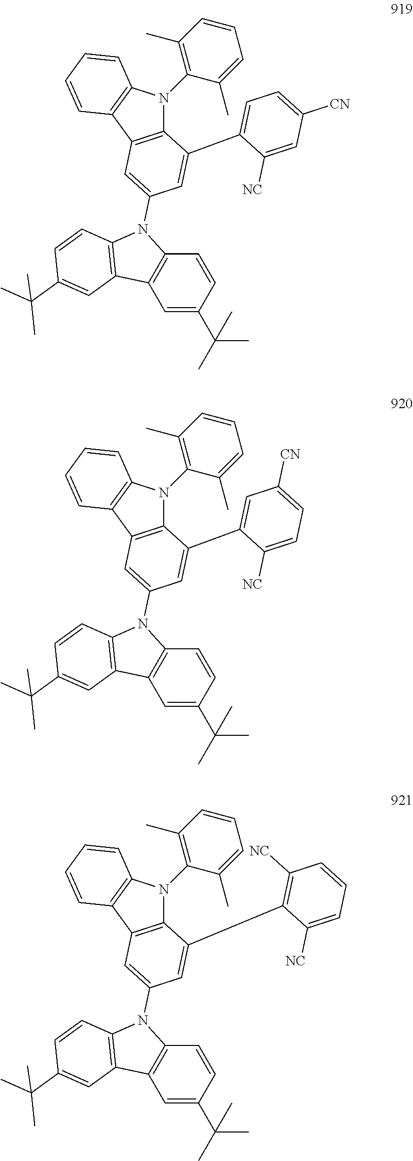

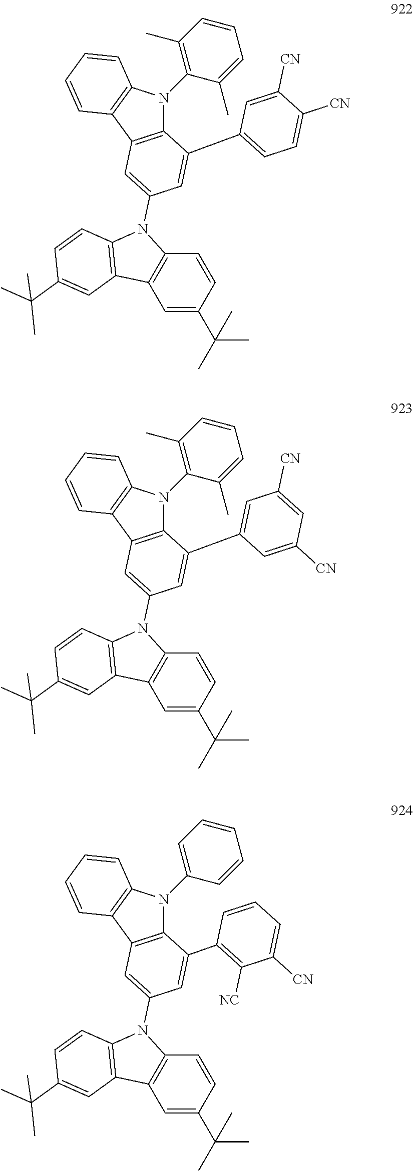 [SMILES: Cc1cccc(C)c1-n1c2ccccc2c2cc(-n3c4ccc(C(C)(C)C)cc4c4cc(C(C)(C)C)ccc43)cc(-c3ccc(C#N)c(C#N)c3)c21 ; Cc1cccc(C)c1-n1c2ccccc2c2cc(-n3c4ccc(C(C)(C)C)cc4c4cc(C(C)(C)C)ccc43)cc(-c3cc(C#N)cc(C#N)c3)c21 ; CC(C)(C)c1ccc2c(c1)c1cc(C(C)(C)C)ccc1n2-c1cc(-c2cccc(C#N)c2C#N)c2c(c1)c1ccccc1n2-c1ccccc1]

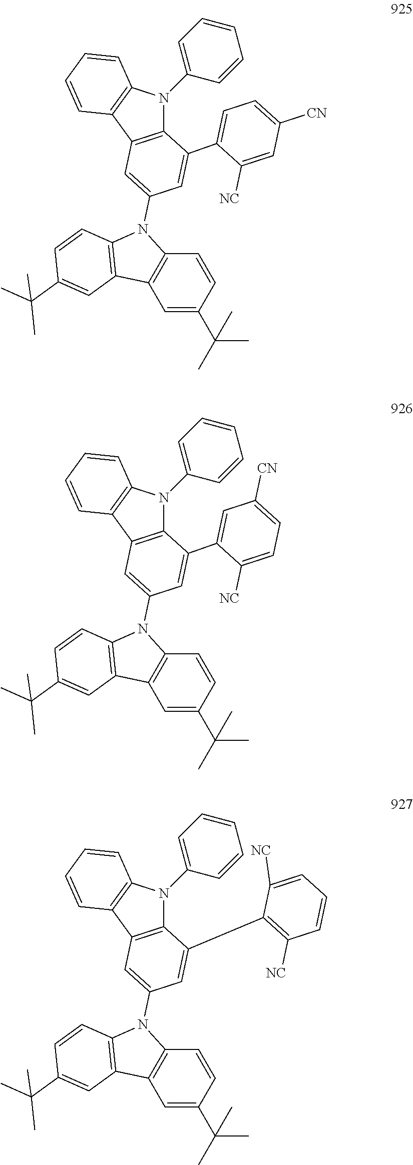

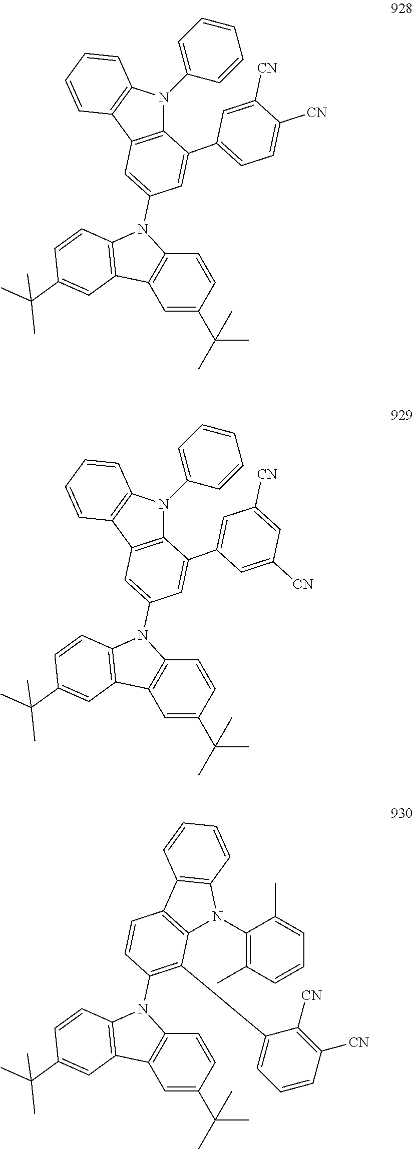 [SMILES: CC(C)(C)c1ccc2c(c1)c1cc(C(C)(C)C)ccc1n2-c1cc(-c2ccc(C#N)c(C#N)c2)c2c(c1)c1ccccc1n2-c1ccccc1 ; CC(C)(C)c1ccc2c(c1)c1cc(C(C)(C)C)ccc1n2-c1cc(-c2cc(C#N)cc(C#N)c2)c2c(c1)c1ccccc1n2-c1ccccc1 ; Cc1cccc(C)c1-n1c2ccccc2c2ccc(-n3c4ccc(C(C)(C)C)cc4c4cc(C(C)(C)C)ccc43)c(-c3cccc(C#N)c3C#N)c21]

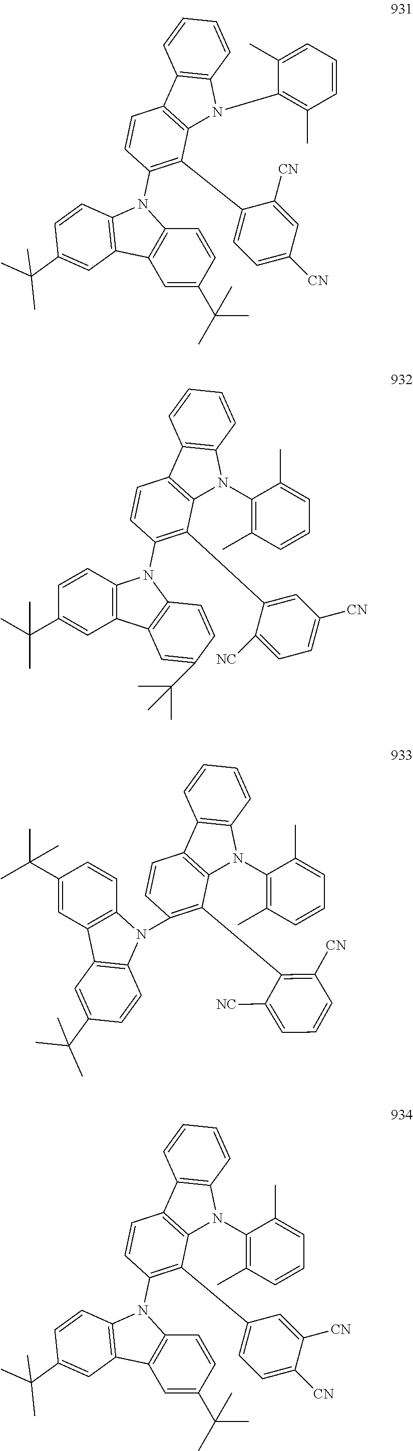

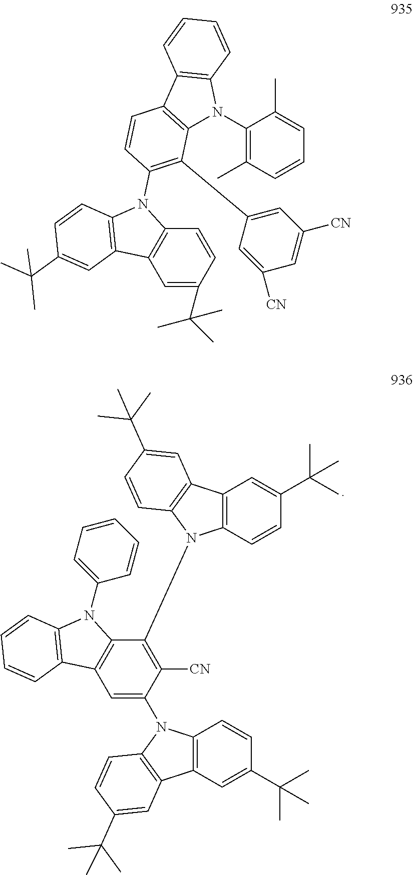 [SMILES: Cc1cccc(C)c1-n1c2ccccc2c2ccc(-n3c4ccc(C(C)(C)C)cc4c4cc(C(C)(C)C)ccc43)c(-c3cc(C#N)cc(C#N)c3)c21 ; CC(C)(C)c1ccc2c(c1)c1cc(C(C)(C)C)ccc1n2-c1cc2c3ccccc3n(-c3ccccc3)c2c(-n2c3ccc(C(C)(C)C)cc3c3cc(C(C)(C)C)ccc32)c1C#N]

The fluorescent compound described above may include the electron donor groups in the number of n1 and the electron acceptor groups in the number of n2 (wherein n1 and n2 may each independently be an integer from 1 to 10), and the electron donor groups in the number of n1 and the electron acceptor groups in the number of n2 may be chemically bonded to each other in random order, provided that chemical bonds between the electron donor groups and the electron acceptor groups may each be a “carbon-carbon single bond”. At least one of the electron donor groups in the number of n1 is an electron donor group represented by Formula 1A as defined herein, and the electron acceptor group is an electron acceptor group represented by Formula 1B as defined herein (wherein “L₁₁” in Formula 1B does not include a “carbazole group” and a “xanthene group”, and “E₁₁” in Formula 1B does not include a “phosphine oxide group” and a “sulfur dioxide group”).

For example, since the electron donor group and the electron acceptor group of Compounds A and D are linked via a relatively weak “nitrogen-carbon single bond”, a bond between the electron donor group and the electron acceptor group of Compounds A and D may be easily broken when Compounds A and D have high excited state energy.

Formula 1A as defined herein) and the electron acceptor group having excellent electron acceptor characteristics (represented by Formula 1B as defined herein) are linked via a “strong” “carbon-carbon single bond”, the fluorescent compound may have a relatively small Stoke's shift (for example, a Stoke's shift in a range of about 0.15 eV to about 0.45 eV, about 0.2 eV to about 0.35 eV, or about 0.21 eV to about 0.29 eV) and energy band gap (energy level). Therefore, an electronic device (for example, an organic light-emitting device) including the fluorescent compound may have a long lifespan due to a reduction in a load generated during the driving of the device. Furthermore, the fluorescent compound may have a sharp photoluminescence (PL) spectrum, for example, a PL spectrum having a relatively small full width at half maximum (FWHM). Therefore, an electronic device (for example, an organic light-emitting device) including the fluorescent compound may have excellent color purity.

In an embodiment, the fluorescent compound may emit blue light. For example, the fluorescent compound may emit blue light having a maximum emission wavelength in a range of about 420 nanometers (nm) to about 490 nm (for example, a range of about 435 nm to about 484 nm), but embodiments of the present disclosure are not limited thereto.

The emission layer of the organic light-emitting device may be implemented according to a first exemplary embodiment, a second exemplary embodiment, or a third exemplary embodiment, based on purposes of the fluorescent compound.

### First Exemplary Embodiment

The first exemplary embodiment is an embodiment in which the fluorescent compound included in the emission layer is used as a fluorescent emitter, that is, the fluorescent compound is a fluorescent emitter.

Therefore, according to the first exemplary embodiment, a proportion of fluorescent emission components of the fluorescent compound with respect to total emission components emitted from the emission layer may be about 80% or more, for example, about 90% or more. For example, the proportion of the fluorescent emission components of the fluorescent compound with respect to the total emission components emitted from the emission layer may be about 95% or more. The fluorescent emission component of the fluorescent compound is the sum of a prompt emission component of the fluorescent compound and a delayed fluorescence component of the fluorescent compound caused by reverse intersystem crossing.

According to the first exemplary embodiment,

the emission layer may consist of (or include only) the fluorescent compound; or

the emission layer may further include a host (wherein the host is not identical to the fluorescent compound).

In the first exemplary embodiment, when the emission layer further includes the host as well as the fluorescent compound, an amount of the fluorescent compound may be about 50 parts by weight or less, for example, about 30 parts by weight or less, based on 100 parts by weight of the emission layer, and an amount of the host in the emission layer may be about 50 parts by weight or more, for example, about 70 parts by weight or more, based on 100 parts by weight of the emission layer, but embodiments of the present disclosure are not limited thereto.

The host in the first exemplary embodiment is the same as described below.

### Second Exemplary Embodiment

The second exemplary embodiment is an embodiment in which the fluorescent compound included in the emission layer is used as a fluorescent host.

Therefore, according to the second exemplary embodiment, the emission layer includes a host and a fluorescent dopant, provided that the fluorescent compound is included in the host, and a proportion of fluorescent emission components of the fluorescent dopant with respect to total emission components emitted from the emission layer may be about 80% or more, for example, about 90% or more (for example, about 95% or more).

In the second exemplary embodiment, an amount of the fluorescent dopant in the emission layer may be about 50 parts by weight or less, for example, about 30 parts by weight or less, based on 100 parts by weight of the emission layer, and an amount of the host in the emission layer may be about 50 parts by weight or more, for example, about 70 parts by weight or more, based on 100 parts by weight of the emission layer, but embodiments of the present disclosure are not limited thereto.

The fluorescent dopant according to the second exemplary embodiment is the same as described below.

The host in the second exemplary embodiment may consist of (or include only) the fluorescent compound, or may further include other known hosts. Embodiments of other known hosts are the same as described below.

### Third Exemplary Embodiment

The third exemplary embodiment is an embodiment in which the fluorescent compound included in the emission layer is used as an auxiliary dopant.

Therefore, according to the third exemplary embodiment, the emission layer may include a host, an auxiliary dopant, and a fluorescent dopant, provided that the fluorescent compound is included in the auxiliary dopant, and the emission layer may satisfy Mathematical Expressions 1 and 2:

E_{T1(HOST)}−E_{T1(AD)}>0.05 eV Mathematical Expression 1

E_{S1(FD)}−E_{S1(AD)}<0 eV. Mathematical Expression 2

In Mathematical Expression 1, E_{T1(HOST)} is triplet energy (eV) of the host, and E_{T1(AD)} is triplet energy (eV) of the auxiliary dopant,

in Mathematical Expression 2, E_{S1(FD)} is singlet energy (eV) of the fluorescent dopant, and E_{S1(AD)} is singlet energy (eV) of the auxiliary dopant, and

E_{T1(HOST)}, E_{T1(AD)}, E_{S1(FD)}, and E_{S1(AD)} may each independently evaluated by using a Density Functional Theory (DFT) method of a Gaussian program that is structurally optimized at a level of B3LYP/6-31G(d,p).

In the third exemplary embodiment, when Mathematical Expression 1 is satisfied (for example, E_{T1(HOST)}−E_{T1(AD)} is greater than 0.10 eV and equal to or less than 0.65 eV), energy of a triplet exciton generated by the auxiliary dopant in the emission layer cannot move toward the host in the emission layer, thus reducing a probability that the triplet exciton will be lost in non-emission paths. Therefore, the organic light-emitting device may have high efficiency.

Also, in the third exemplary embodiment, when Mathematical Expression 2 is satisfied (for example, E_{S1(FD)}−E_{S1(AD)} is greater than −0.4 eV and equal to or less than −0.05 eV), energy of singlet excitons generated by the auxiliary dopant in the emission layer may rapidly move to the fluorescent dopant. Therefore, substantially, emission occurs only in the fluorescent dopant in the emission layer of the organic light-emitting device, thereby embodying a fluorescent dopant-based fluorescent PL spectrum with excellent color purity. In addition, fluorescent emission having a relatively short exciton lifespan may occur, and accordingly, an efficiency-conversion phenomenon under high luminance (also called a roll-off phenomenon), which may occur due to an interaction between a plurality of excitons (exciton-exciton interaction) or an interaction between an exciton and a charge (hole or electron) (exciton-polaron interaction), is suppressed to produce an organic light-emitting device having high efficiency. Furthermore, the auxiliary dopant has a short exciton lifespan, thus reducing a probability of chemical or physical deterioration which may occur in an exciton state of the auxiliary dopant. Therefore, the organic light-emitting device satisfying Mathematical Expression 2 may have improved durability.

In the second exemplary embodiment, an amount of the fluorescent dopant in the emission layer may be about 50 parts by weight or less, for example, about 30 parts by weight or less, based on 100 parts by weight of the emission layer, an amount of the host in the emission layer may be about 50 parts by weight or more, for example, about 70 parts by weight or more, based on 100 parts by weight of the emission layer, and an amount of the auxiliary dopant may be about 30 parts by weight or less, for example, about 20 parts by weight or less, based on 100 parts by weight of the emission layer, but embodiments of the present disclosure are not limited thereto.

The host and the fluorescent dopant according to the third exemplary embodiment are the same as described below.

The emission layer according to the second exemplary embodiment may include i) the fluorescent compound (host) as defined herein and ii) the fluorescent dopant (fluorescent emitter), and the emission layer according to the third exemplary embodiment may include i) the host, ii) the fluorescent dopant (fluorescent emitter), and iii) the fluorescent compound (auxiliary dopant) as defined herein. Therefore, in the emission layers according to the second and third exemplary embodiments, energy transfer from the fluorescent compound to the fluorescent dopant (fluorescent emitter) may be performed based on a Forster energy transfer mechanism. The fluorescent compound has a relatively small Stoke's shift and energy band gap (energy level), thus increasing an overlap region between a PL spectrum of the fluorescent compound acting as an energy donor and an absorption spectrum of the fluorescent dopant (fluorescent emitter) acting as an energy acceptor. Therefore, since energy transfer for light emission in the emission layers according to the second and third exemplary embodiments may be effectively achieved, the organic light-emitting device including the emission layer may have both high efficiency and a long lifespan.

For example, the emission layer of the organic light-emitting devices according to the first and third exemplary embodiments may emit blue light (for example, blue light having a maximum emission wavelength in a range of about 420 nm to about 490 nm or about 431 nm to about 481 nm), but embodiments of the present disclosure are not limited thereto.

For example, the emission layer of the organic light-emitting devices according to the first and third exemplary embodiments may emit blue light having a CIE y coordinate in a range of about 0.04 to about 0.45 or about 0.10 to about 0.37, but embodiments of the present disclosure are not limited thereto.

The hosts according to the first and third exemplary embodiments may be selected from known fluorescent hosts.

For example, the host may have a triplet energy level of 2.9 eV or more, for example, a triplet energy level greater than 2.9 eV and equal to or less than 4.5 eV. Since energy transfer from the host to the fluorescent emitter, and/or the fluorescent dopant may be effectively achieved, the organic light-emitting device may have high efficiency.

For example, the host may include at least one compound selected from a fluorene-containing compound, a carbazole-containing compound, a dibenzofuran-containing compound, a dibenzothiophene-containing compound, an indenocarbazole-containing compound, an indolocarbazole-containing compound, a benzofurocarbazole-containing compound, a benzothienocarbazole-containing compound, an acridine-containing compound, a dihydroacridine-containing compound, a triindolobenzene-containing compound, a pyridine-containing compound, a pyrimidine-containing compound, a triazine-containing compound, a silicon-containing compound, a cyano group-containing compound, a phosphine oxide-containing compound, and a sulfoxide-containing compound, but embodiments of the present disclosure are not limited thereto.

In one or more embodiments, the host may include a compound including at least one carbazole ring and at least one cyano group.

For example, the host may be selected from compounds represented by Formulae 11-1 to 11-3, but embodiments of the present disclosure are not limited thereto:

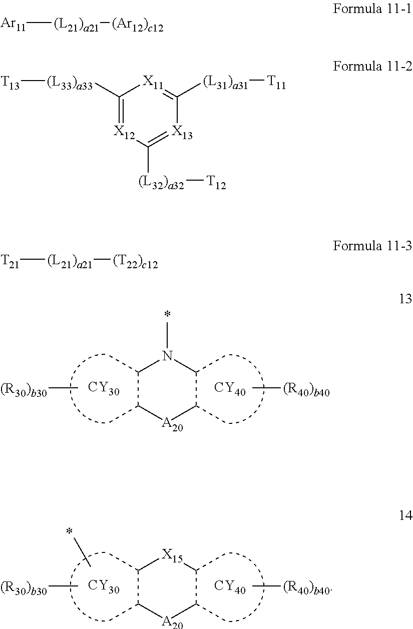

In Formulae 11-1 to 11-3, 13, and 14,

Ar₁₁ and Ar₁₂ may each independently be selected from groups represented by Formulae 13 and 14,

X₁₅ may be N(R₂₀₀), O, or S,

X₁₁ may be N or C(T₁₄), X₁₂ may be N or C(T₁₅), and X₁₃ may be N or C(T₁₆), provided that at least one of X₁₁ to X₁₃ is N,

T₂₁ and T₂₂ may each independently be selected from *-(L₂₁)ₐ₂₁-Si(Q₄₁)(Q₄₂)(Q₄₃) and *-(L₂₁)ₐ₂₁-P(═O)(Q₅₁)(Q₅₂),

L₂₁ and L₃₁ to L₃₃ may each independently be selected from:

a single bond, O, S, Si(Q₆₁)(Q₆₂), a phenylene group, a pyridinylene group, a pyrimidinylene group, a pyrazinylene group, a pyridazinylene group, a triazinylene group, a naphthylene group, a fluorenylene group, a carbazolylene group, a dibenzofuranylene group, and a dibenzothiophenylene group; and

a phenylene group, a pyridinylene group, a pyrimidinylene group, a pyrazinylene group, a pyridazinylene group, a triazinylene group, a naphthylene group, a fluorenylene group, a carbazolylene group, a dibenzofuranylene group, and a dibenzothiophenylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, —CF₃, —CF₂H, —CFH₂, a phenyl group, a phenyl group substituted with a cyano group, a biphenyl group, a terphenyl group, a naphthyl group, a pyridinyl group, a pyrimidinyl group, a pyrazinyl group, a pyridazinyl group, a triazinyl group, a fluorenyl group, a carbazolyl group, a dibenzofuranyl group, a dibenzothiophenyl group, and —Si(Q₇₁)(Q₇₂)(Q₇₃),

a21 and a31 to a33 may each independently be an integer from 0 to 5, wherein, when a21 is two or more, two or more groups L₂₁ may be identical to or different from each other, when a31 is two or more, two or more groups L₃₁ may be identical to or different from each other, when a32 is two or more, two or more groups L₃₂ may be identical to or different from each other, and when a33 is two or more, two or more groups L₃₃ may be identical to or different from each other,

CY₃₀ and CY₄₀ may each independently be selected from a benzene group, a naphthalene group, a fluorene group, a carbazole group, a benzocarbazole group, an indolocarbazole group, a dibenzofuran group, and a dibenzothiophene group,

A₂₀ may be selected from:

a single bond, a C₁-C₄ alkylene group, and a C₂-C₄ alkenylene group; and

a C₁-C₄ alkylene group and a C₂-C₄ alkenylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a biphenyl group, a terphenyl group, a naphthyl group, a pyridinyl group, a pyrimidinyl group, a pyrazinyl group, a pyridazinyl group, a triazinyl group, a fluorenyl group, a carbazolyl group, a dibenzofuranyl group, a dibenzothiophenyl group, and —Si(Q₈₁)(Q₈₂)(Q₈₃),

T₁₁ to T₁₆, R₂₀₀, R₃₀, and R₄₀ may each independently be selected from hydrogen, deuterium, —F, —Cl, —Br, —I, a hydroxyl group, a cyano (CN) group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₇-C₆₀ arylalkyl group, a substituted or unsubstituted C₁-C₆₀ heteroaryl group, a substituted or unsubstituted C₂-C₆₀ heteroaryloxy group, a substituted or unsubstituted C₂-C₆₀ heteroarylthio group, a substituted or unsubstituted C₃-C₆₀ heteroarylalkyl group, a substituted or unsubstituted monovalent non-aromatic condensed polycyclic group, a substituted or unsubstituted monovalent non-aromatic condensed heteropolycyclic group, and —Si(Q₉₁)(Q₉₂)(Q₉₃),

b30 and b40 may each independently be an integer from 0 to 10,

c12 may be 0, 1, 2, or 3,

“*” indicates a binding site to a neighboring atom,

at least one substituent of the substituted C₁-C₆₀ alkyl group, the substituted C₂-C₆₀ alkenyl group, the substituted C₂-C₆₀ alkynyl group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₇-C₆₀ arylalkyl group, the substituted C₁-C₆₀ heteroaryl group, the substituted C₂-C₆₀ heteroaryloxy group, the substituted C₂-C₆₀ heteroarylthio group, the substituted C₃-C₆₀ heteroarylalkyl group, the substituted monovalent non-aromatic condensed polycyclic group, and the substituted monovalent non-aromatic condensed heteropolycyclic group may be selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, and —Si(Q₁₀₁)(Q₁₀₂)(Q₁₀₃), and

Q₄₁ to Q₄₃, Q₅₁ to Q₅₂, Q₆₁ to Q₆₂, Q₇₁ to Q₇₃, Q₈₁ to Q₈₃, Q₉₁ to Q₉₃, and Q₁₀₁ to Q₁₀₃ may each independently be selected from hydrogen, deuterium, a C₁-C₆₀ alkyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

For example, the host may include at least one compound selected from Compounds H1 to H19, but embodiments of the present disclosure are not limited thereto:

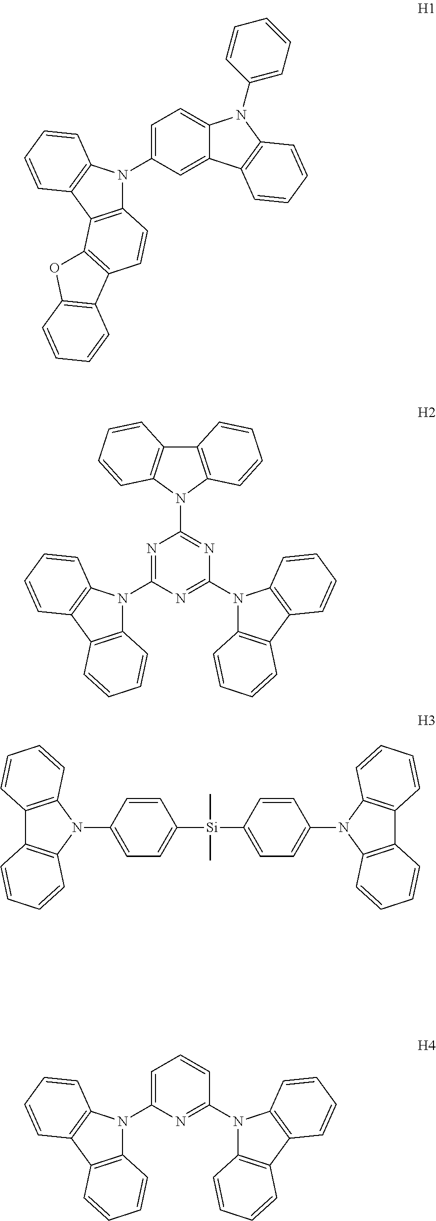 [SMILES: c1ccc(-n2c3ccccc3c3cc(-n4c5ccccc5c5c6oc7ccccc7c6ccc54)ccc32)cc1 ; c1ccc2c(c1)c1ccccc1n2-c1nc(-n2c3ccccc3c3ccccc32)nc(-n2c3ccccc3c3ccccc32)n1 ; C[Si](C)(c1ccc(-n2c3ccccc3c3ccccc32)cc1)c1ccc(-n2c3ccccc3c3ccccc32)cc1 ; c1cc(-n2c3ccccc3c3ccccc32)nc(-n2c3ccccc3c3ccccc32)c1]

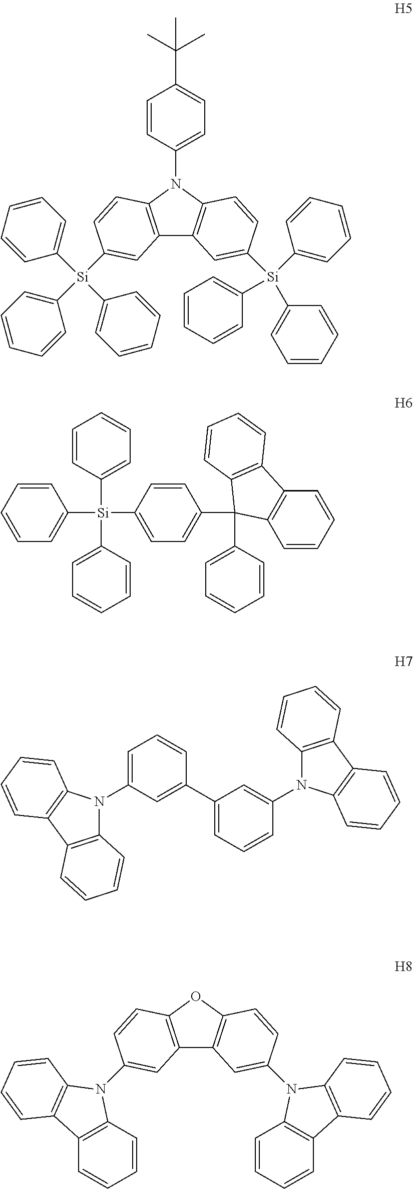 [SMILES: CC(C)(C)c1ccc(-n2c3ccc([Si](c4ccccc4)(c4ccccc4)c4ccccc4)cc3c3cc([Si](c4ccccc4)(c4ccccc4)c4ccccc4)ccc32)cc1 ; c1ccc(C2(c3ccc([Si](c4ccccc4)(c4ccccc4)c4ccccc4)cc3)c3ccccc3-c3ccccc32)cc1 ; c1cc(-c2cccc(-n3c4ccccc4c4ccccc43)c2)cc(-n2c3ccccc3c3ccccc32)c1 ; c1ccc2c(c1)c1ccccc1n2-c1ccc2oc3ccc(-n4c5ccccc5c5ccccc54)cc3c2c1]

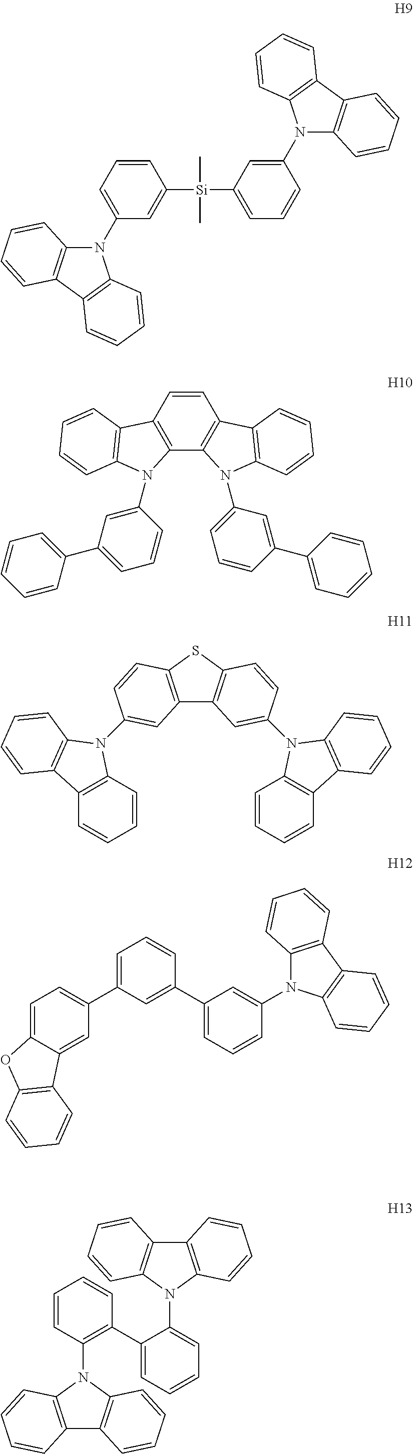 [SMILES: C[Si](C)(c1cccc(-n2c3ccccc3c3ccccc32)c1)c1cccc(-n2c3ccccc3c3ccccc32)c1 ; c1ccc(-c2cccc(-n3c4ccccc4c4ccc5c6ccccc6n(-c6cccc(-c7ccccc7)c6)c5c43)c2)cc1 ; c1ccc2c(c1)c1ccccc1n2-c1ccc2sc3ccc(-n4c5ccccc5c5ccccc54)cc3c2c1 ; c1cc(-c2cccc(-n3c4ccccc4c4ccccc43)c2)cc(-c2ccc3oc4ccccc4c3c2)c1 ; c1ccc(-n2c3ccccc3c3ccccc32)c(-c2ccccc2-n2c3ccccc3c3ccccc32)c1]

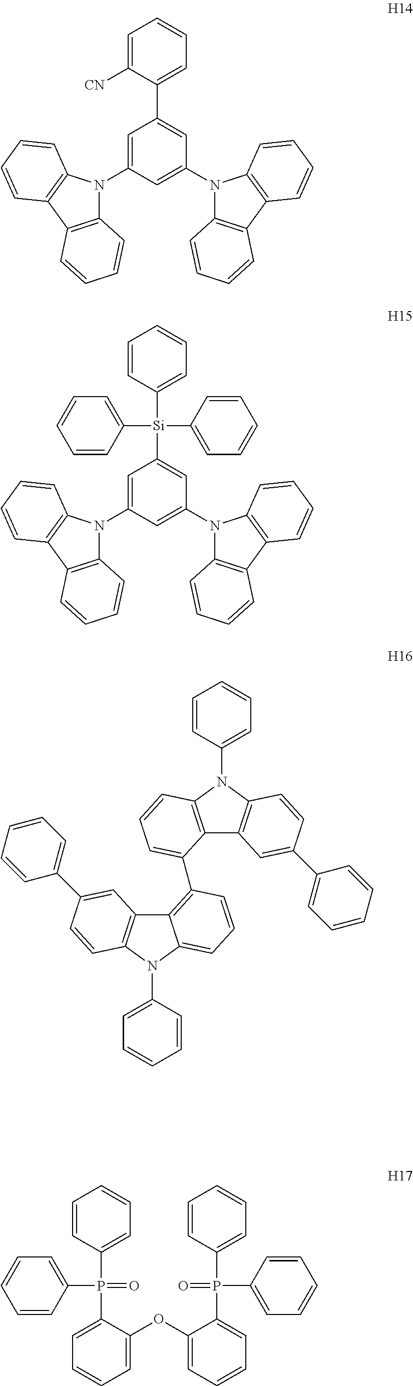

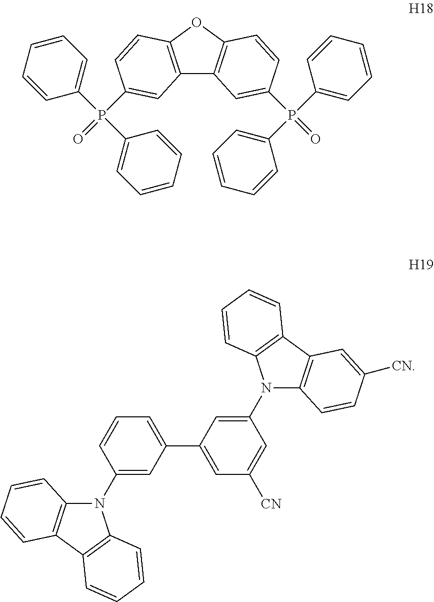 [SMILES: O=P(c1ccccc1)(c1ccccc1)c1ccc2oc3ccc(P(=O)(c4ccccc4)c4ccccc4)cc3c2c1 ; N#Cc1cc(-c2cccc(-n3c4ccccc4c4ccccc43)c2)cc(-n2c3ccccc3c3cc(C#N)ccc32)c1]

In the second exemplary embodiment and the third exemplary embodiment, the fluorescent dopant may be selected from a condensed polycyclic compound and a styryl-based compound.

For example, the fluorescent dopant may include one selected from a naphthalene-containing core, a fluorene-containing core, a spiro-bifluorene-containing core, a benzofluorene-containing core, a dibenzofluorene-containing core, a phenanthrene-containing core, an anthracene-containing core, a fluoranthene-containing core, a triphenylene-containing core, a pyrene-containing core, a chrysene-containing core, a naphthacene-containing core, a picene-containing core, a perylene-containing core, a pentaphene-containing core, an indenoanthracene-containing core, a tetracene-containing core, a bisanthracene-containing core, and cores represented by Formulae 501-1 to 501-18, but embodiments of the present disclosure are not limited thereto:

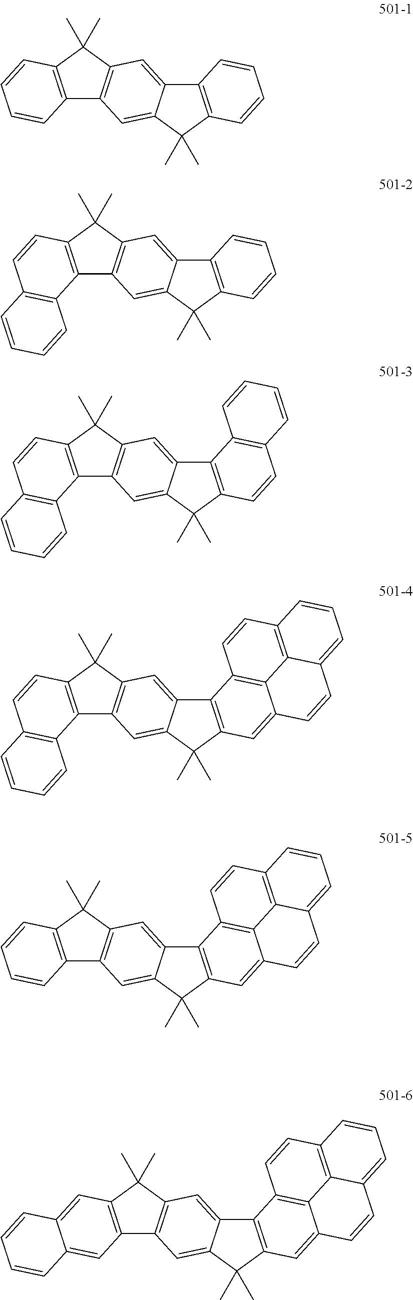

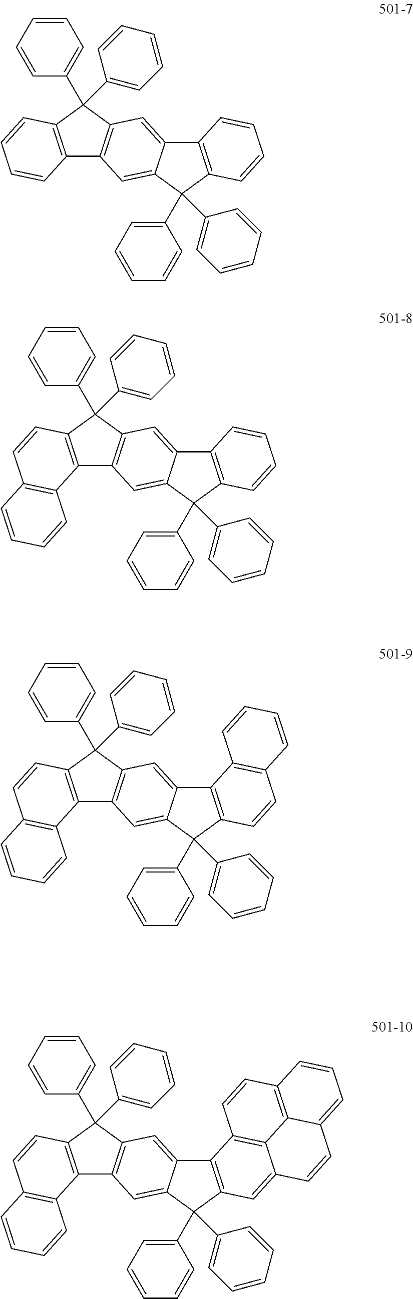

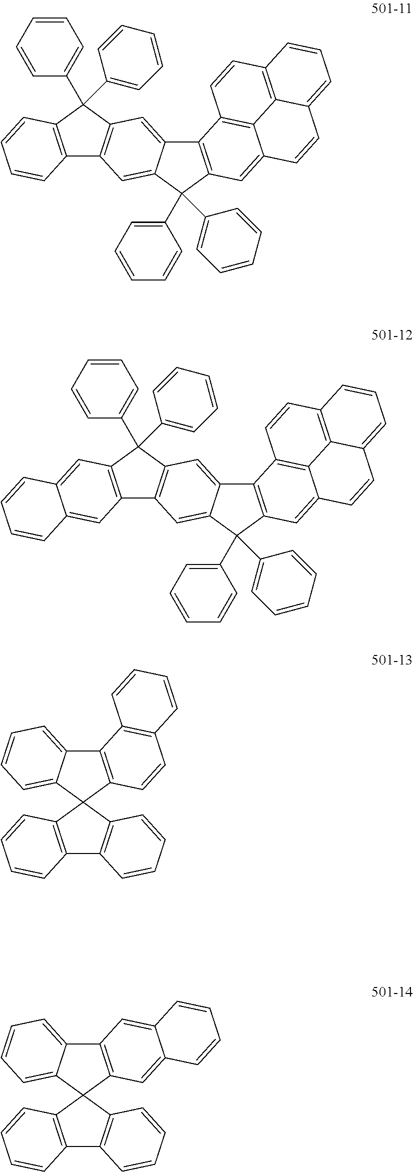

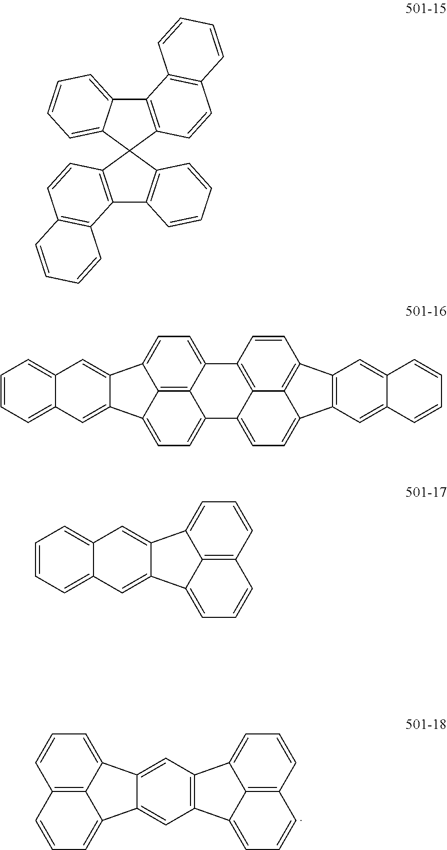

Alternatively, the fluorescent dopant may be selected from a styryl-amine-based compound and a styryl-carbazole-based compound, but embodiments of the present disclosure are not limited thereto.

In one or more embodiments, the fluorescent dopant may be selected from compounds represented by Formula 501:

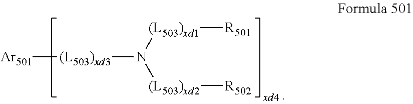

In Formula 50,

Ar₅₀₁ may be selected from:

a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, and groups represented by Formulae 501-1 to 501-18; and

a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, and groups represented by Formulae 501-1 to 501-18, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, and —Si(Q₅₀₁)(Q₅₀₂)(Q₅₀₃) (wherein Q₅₀₁ to Q₅₀₃ may each independently be selected from hydrogen, a C₁-C₆₀ alkyl group, a C₁-C₆₀ alkoxy group, a C₆-C₆₀ aryl group, a C₁-C₆₀ heteroaryl group, a monovalent non-aromatic condensed polycyclic group, and a monovalent non-aromatic condensed heteropolycyclic group),

L₅₀ to L₅₀₃ may each independently be selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

R₅₀₁ and R₅₀₂ may each independently be selected from:

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, a triazinyl group, a dibenzofuranyl group, and a dibenzothiophenyl group; and

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, and a dibenzothiophenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, and a dibenzothiophenyl group,

xd1 to xd3 may each independently be selected from 0, 1, 2, and 3, and

xd4 may be selected from 0, 1, 2, 3, 4, 5, and 6.

For example, in Formula 50,

Ar₅₀₁ may be selected from:

a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, and groups represented by Formulae 501-1 to 501-18; and

a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, and groups represented by Formulae 501-1 to 501-18, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a dibenzofuranyl group, a dibenzothiophenyl group, a carbazolyl group, a pyridinyl group, a pyrimidinyl group, a triazinyl group, a quinolinyl group, an isoquinolinyl group, and —Si(Q₅₀₁)(Q₅₀₂)(Q₅₀₃) (wherein Q₅₀₁ to Q₅₀₃ may each independently be selected from hydrogen, a C₁-C₂₀ alkyl group, a C₁-C₂₀ alkoxy group, a phenyl group, a biphenyl group, a terphenyl group, and a naphthyl group),

L₅₀₁ to L₅₀₃ are the same as described in connection with L₂₁,

xd1 to xd3 may each independently be selected from 0, 1, and 2,

xd4 may be selected from 0, 1, 2, and 3, but embodiments of the present disclosure are not limited thereto.

Alternatively, the fluorescent dopant may include a compound represented by one of Formulae 502-1 to 502-5:

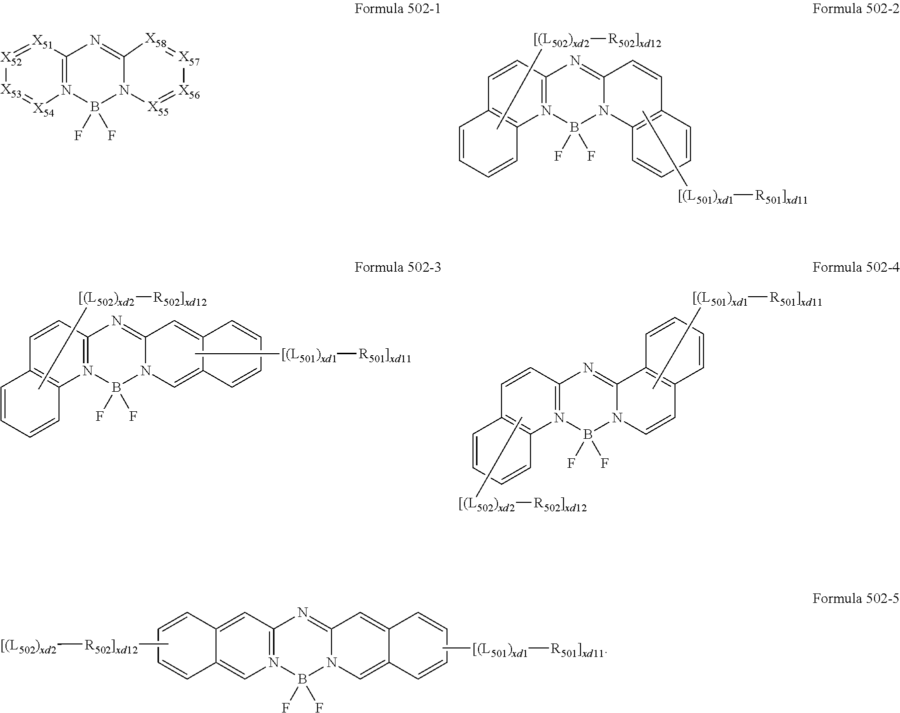

In Formulae 502-1 to 502-5,

X₅₁ may be N or C-[(L₅₀₁)_{xd1}-R₅₀₁], X₅₂ may be N or C-[(L₅₀₂)_{xd2}-R₅₀₂], X₅₃ may be N or C-[(L₅₀₃)_{xd3}-R₅₀₃], X₅₄ may be N or C-[(L₅₀₄)_{xd4}-R₅₀₄], X₅₅ may be N or C-[(L₅₀₅)_{xd5}-R₅₀₅], X₅₆ may be N or C-[(L₅₀₆)_{xd6}-R₅₀₆], X₅₇ may be N or C-[(L₅₀₇)_{xd7}-R₅₀₇], and X₅₈ may be N or C-[(L₅₀₈)_{xd8}-R₅₀₈],

L₅₀₁ to L₅₀₈ are the same as described in connection with L₅₀₁ in Formula 501,

xd1 to xd8 are the same as described in connection with xd1 in Formula 501,

R₅₀₁ to R₅₀₈ may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, a triazinyl group, a dibenzofuranyl group, and a dibenzothiophenyl group; and

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, and a dibenzothiophenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, and a dibenzothiophenyl group,

xd11 and xd12 may each independently be an integer from 0 to 5,

two of R₅₀₁ to R₅₀₄ may optionally be linked to form a saturated or unsaturated ring, and

two of R₅₀₅ to R₅₀₈ may optionally be linked to form a saturated or unsaturated ring.

The fluorescent dopant may include, for example, at least one compound selected from Compounds FD(1) to FD(16) and FD1 to FD13:

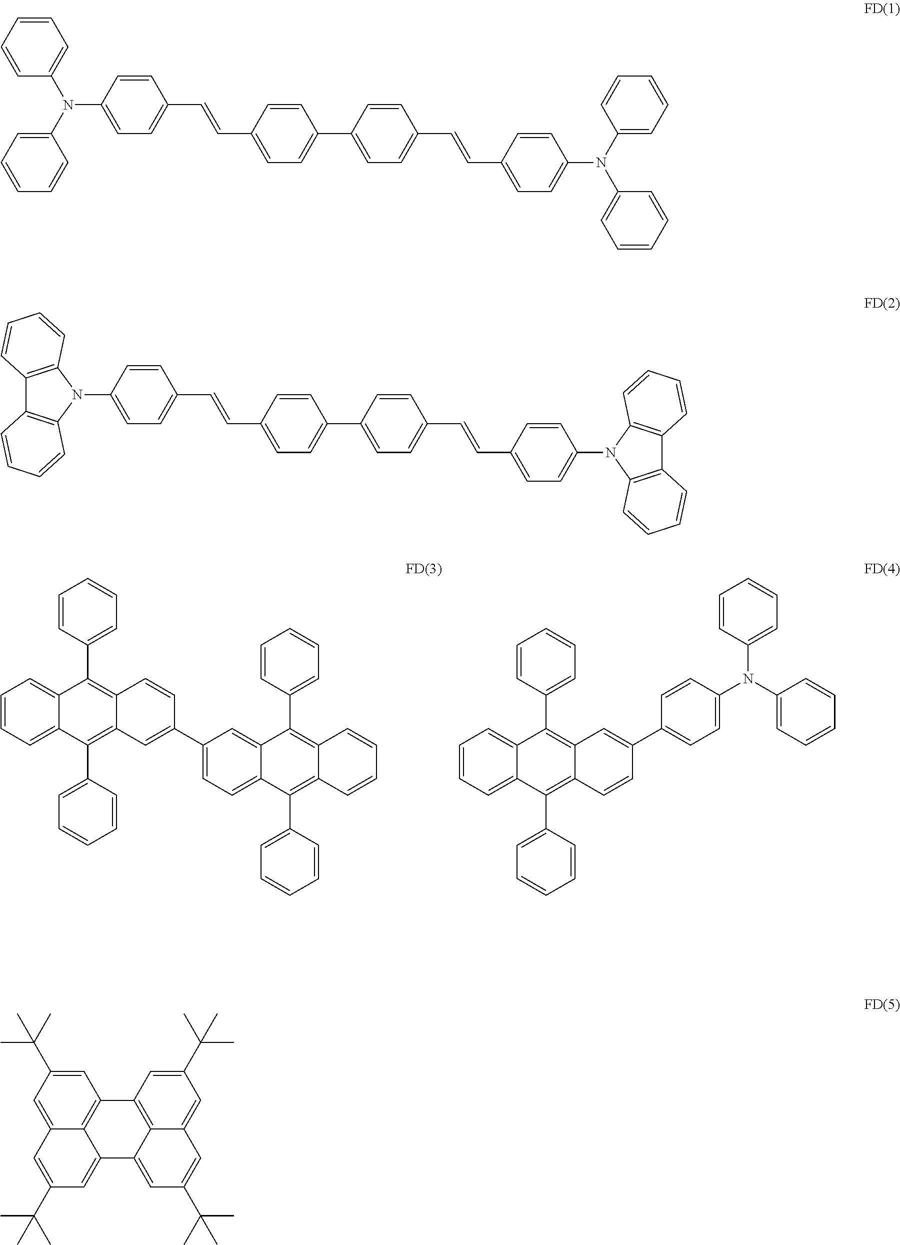

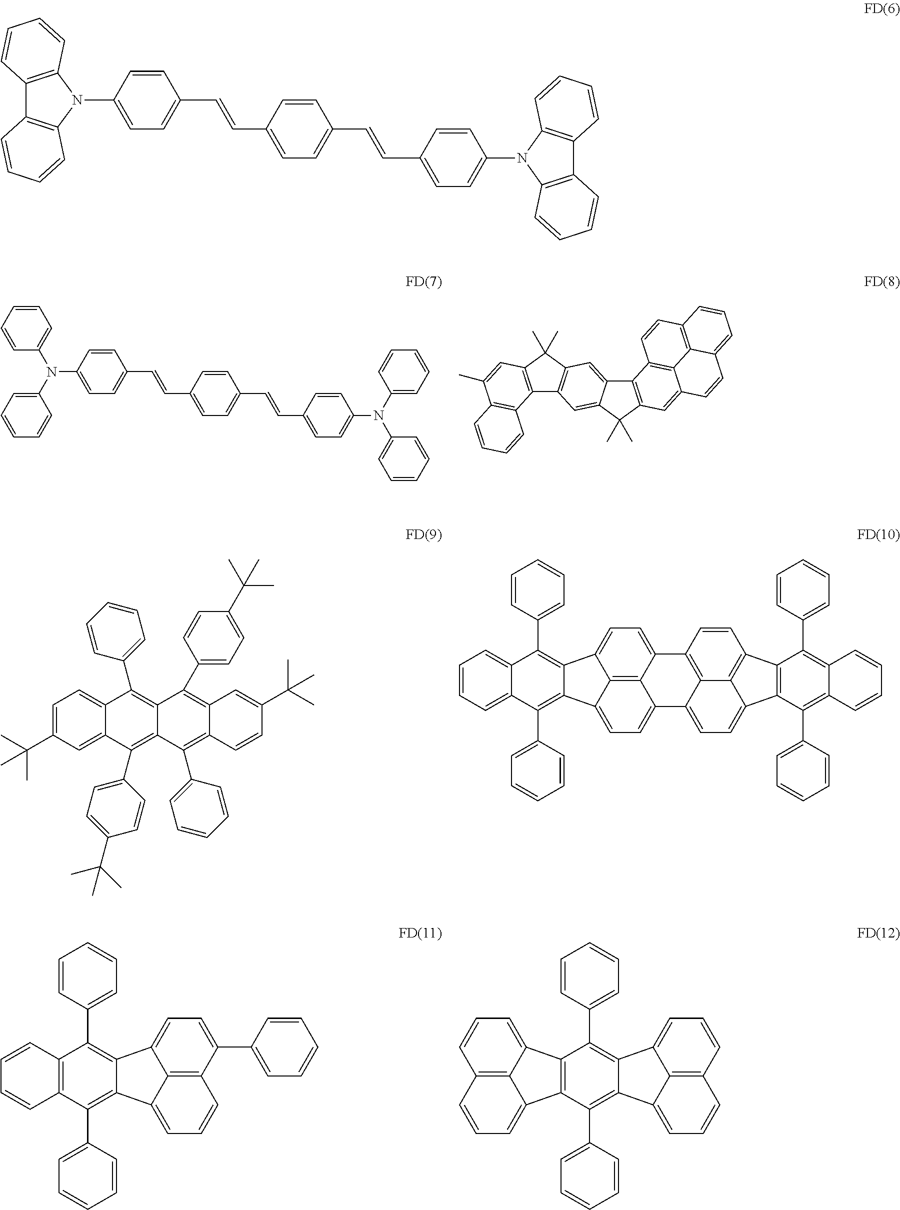

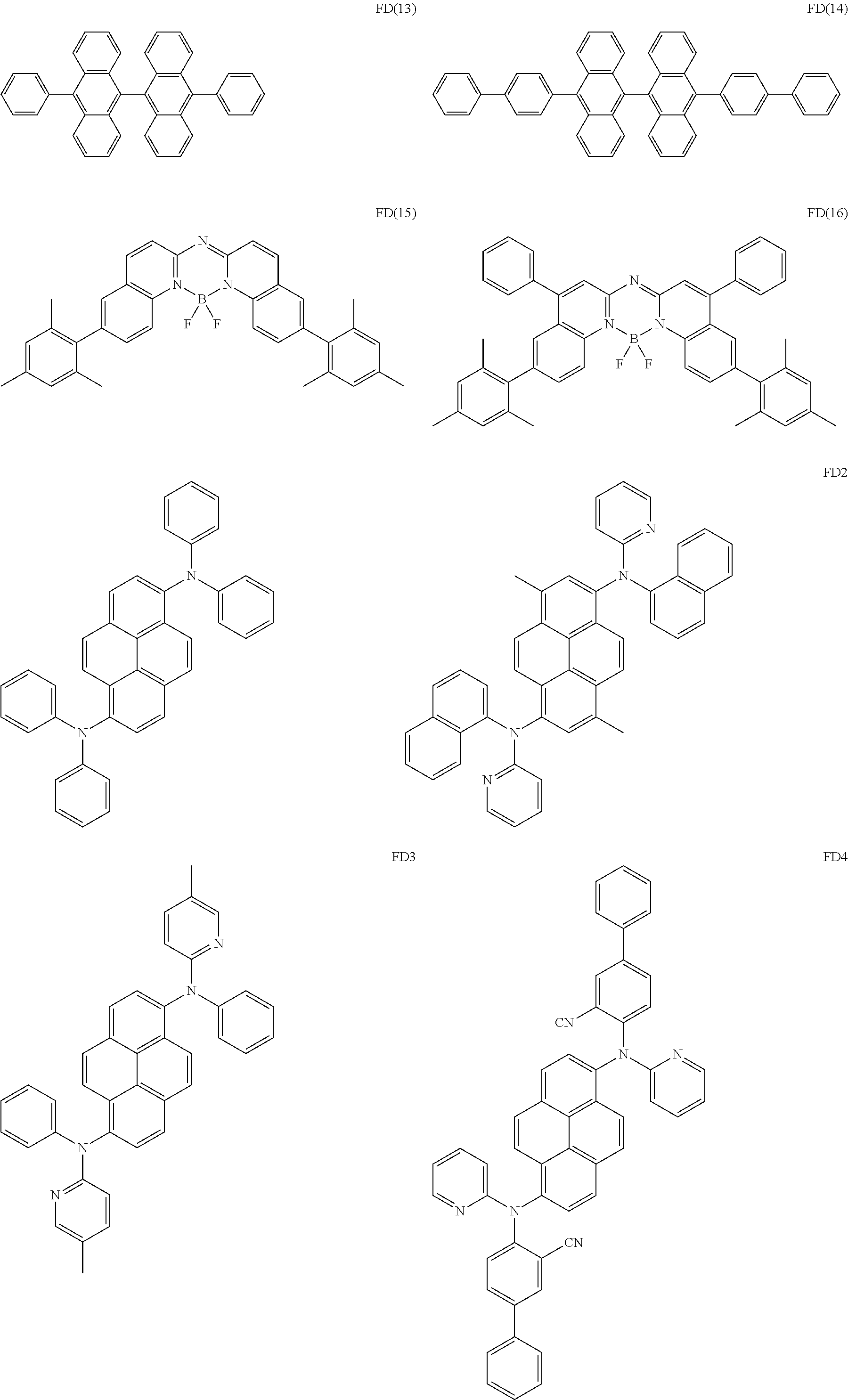

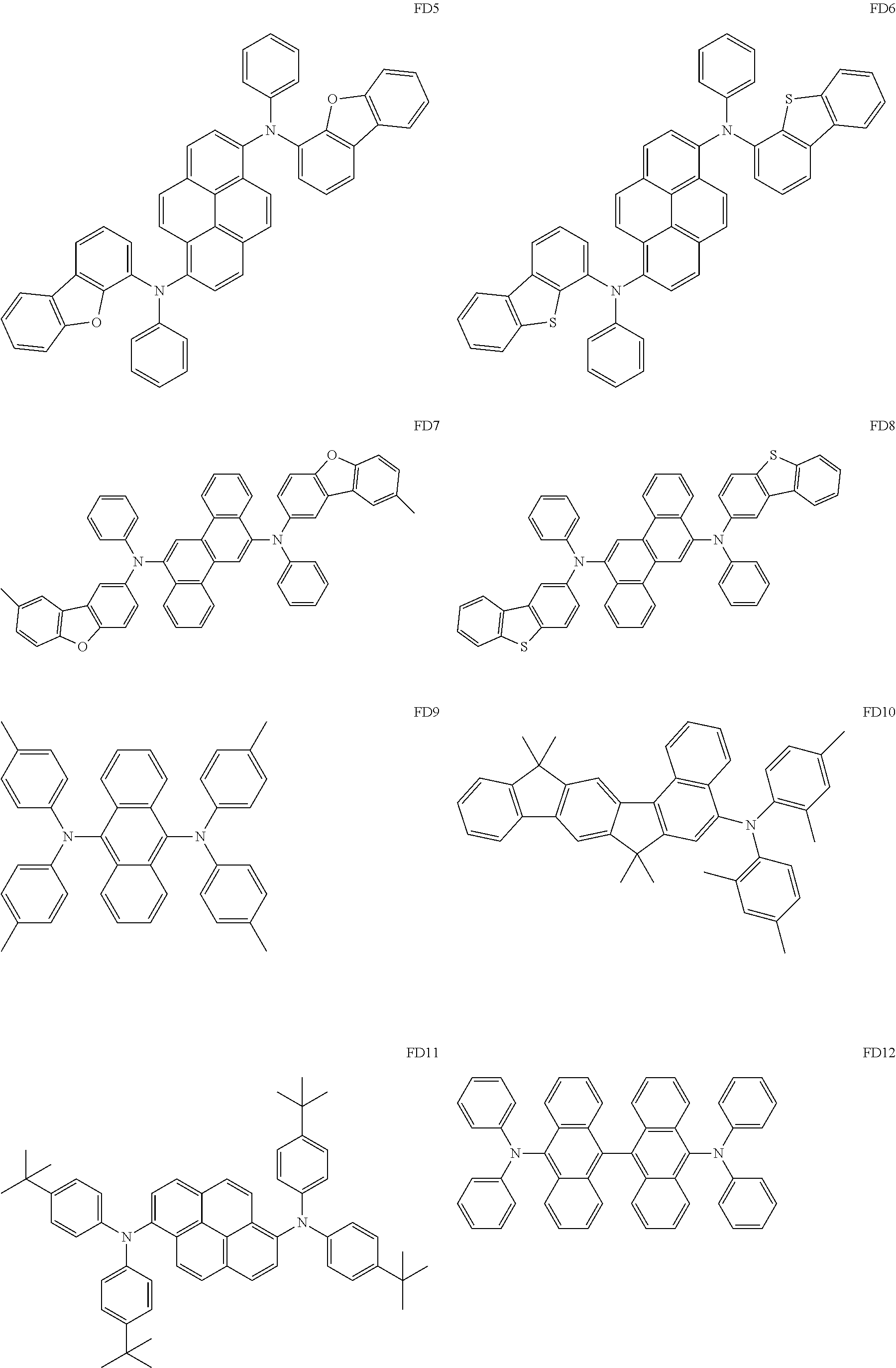

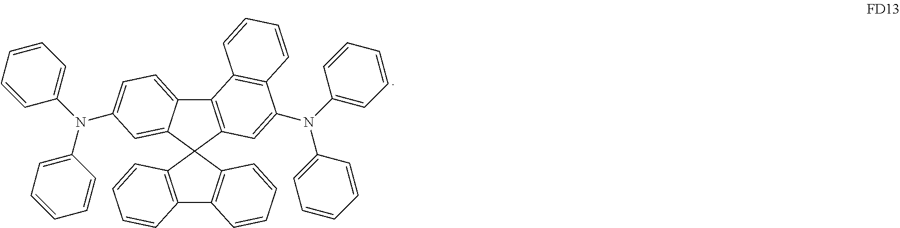 [SMILES: c1ccc(N(c2ccccc2)c2ccc3c(c2)C2(c4ccccc4-c4ccccc42)c2cc(N(c4ccccc4)c4ccccc4)c4ccccc4c2-3)cc1]

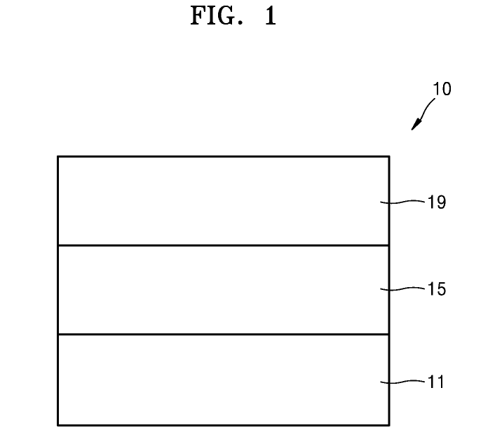

FIG. 1 is a schematic view of an organic light-emitting device 10 according to an embodiment. Hereinafter, the structure of an organic light-emitting device according to an embodiment and a method of manufacturing an organic light-emitting device according to an embodiment will be described in connection with FIG. 1. The organic light-emitting device 10 includes a first electrode 11, an organic layer 15, and a second electrode 19, which are sequentially stacked in this stated order.

A substrate may be additionally disposed under the first electrode 11 or above the second electrode 19. For use as the substrate, any substrate that is used in general organic light-emitting devices may be used, and the substrate may be a glass substrate or a transparent plastic substrate, each having excellent mechanical strength, thermal stability, transparency, surface smoothness, ease of handling, and water resistance.

The first electrode 11 may be formed by depositing or sputtering a material for forming the first electrode 11 on the substrate. The first electrode 11 may be an anode. The material for forming the first electrode 11 may be selected from materials with a high work function to facilitate hole injection. The first electrode 11 may be a reflective electrode, a semi-transmissive electrode, or a transmissive electrode. The material for forming the first electrode may be, for example, indium tin oxide (ITO), indium zinc oxide (IZO), tin oxide (SnO₂), and zinc oxide (ZnO). In one or more embodiments, magnesium (Mg), aluminum (Al), aluminum-lithium (Al—Li), calcium (Ca), magnesium-indium (Mg—In), or magnesium-silver (Mg—Ag) may be used as the material for forming the first electrode.

The first electrode 11 may have a single-layered structure or a multi-layered structure including two or more layers. For example, the first electrode 11 may have a three-layered structure of ITO/Ag/ITO, but the structure of the first electrode 110 is not limited thereto.

The organic layer 15 is disposed on the first electrode 11.

The organic layer 15 may include a hole transport region, an emission layer, and an electron transport region.

The hole transport region may be disposed between the first electrode 11 and the emission layer.

The hole transport region may include at least one selected from a hole injection layer, a hole transport layer, an electron blocking layer, and a buffer layer.

The hole transport region may include only either a hole injection layer or a hole transport layer. In one or more embodiments, the hole transport region may have a hole injection layer/hole transport layer structure or a hole injection layer/hole transport layer/electron blocking layer structure, which are sequentially stacked in this stated order from the first electrode 11.

A hole injection layer may be formed on the first electrode 11 by using one or more suitable methods selected from vacuum deposition, spin coating, casting, or Langmuir-Blodgett (LB) deposition.

When a hole injection layer is formed by vacuum deposition, the deposition conditions may vary according to a material that is used to form the hole injection layer, and the structure and thermal characteristics of the hole injection layer. For example, the deposition conditions may include a deposition temperature of about 100° C. to about 500° C., a vacuum pressure of about 10⁻⁸ torr to about 10⁻³ torr, and a deposition rate of about 0.01 Å/sec to about 100 Å/sec. However, the deposition conditions are not limited thereto.

When the hole injection layer is formed using spin coating, coating conditions may vary according to the material used to form the hole injection layer, and the structure and thermal properties of the hole injection layer. For example, a coating speed may be from about 2,000 revolutions per minute (rpm) to about 5,000 rpm, and a temperature at which a heat treatment is performed to remove a solvent after coating may be from about 80° C. to about 200° C. However, the coating conditions are not limited thereto.

Conditions for forming a hole transport layer and an electron blocking layer may be understood by referring to conditions for forming the hole injection layer.

The hole transport region may include at least one selected from m-MTDATA, TDATA, 2-TNATA, NPB, β-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzene sulfonic acid (PANI/DBSA), poly(3,4-ethylenedioxythiophene)/poly(4-styrene sulfonate) (PEDOT/PSS), polyaniline/camphor sulfonic acid (PANI/CSA), polyaniline/poly(4-styrene sulfonate) (PANI/PSS), a compound represented by Formula 201 below, and a compound represented by Formula 202:

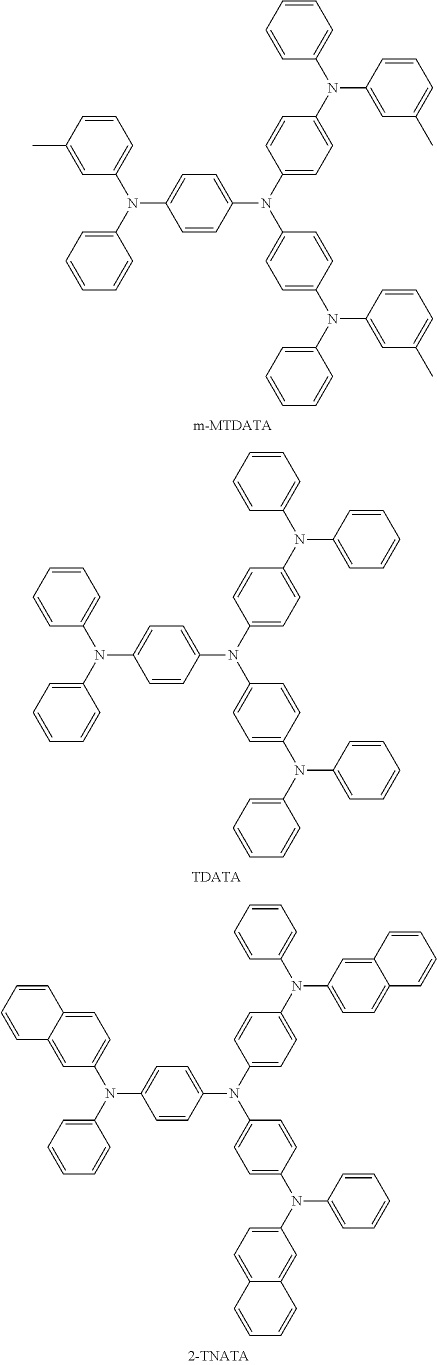

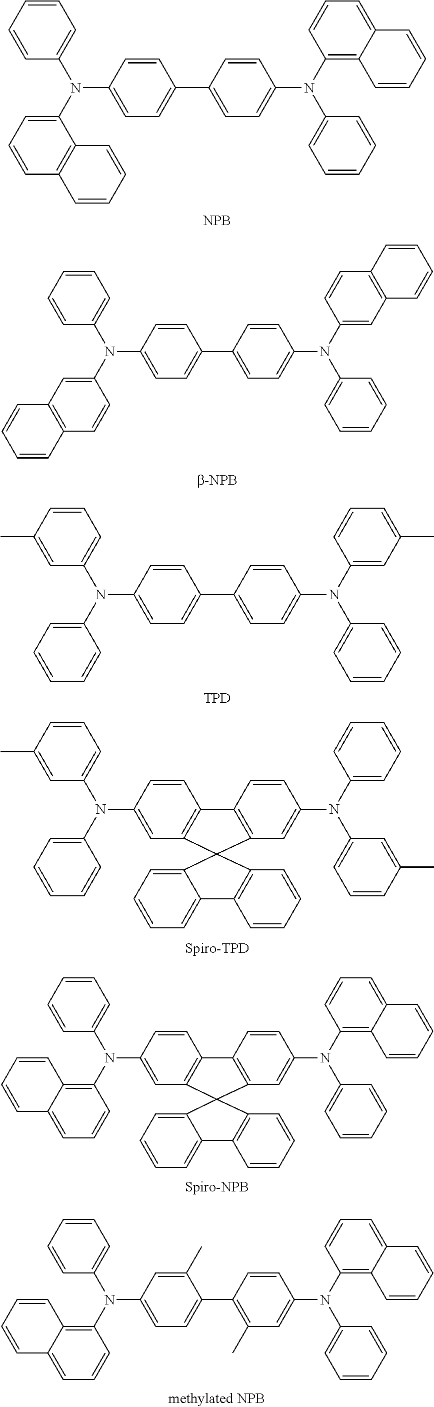

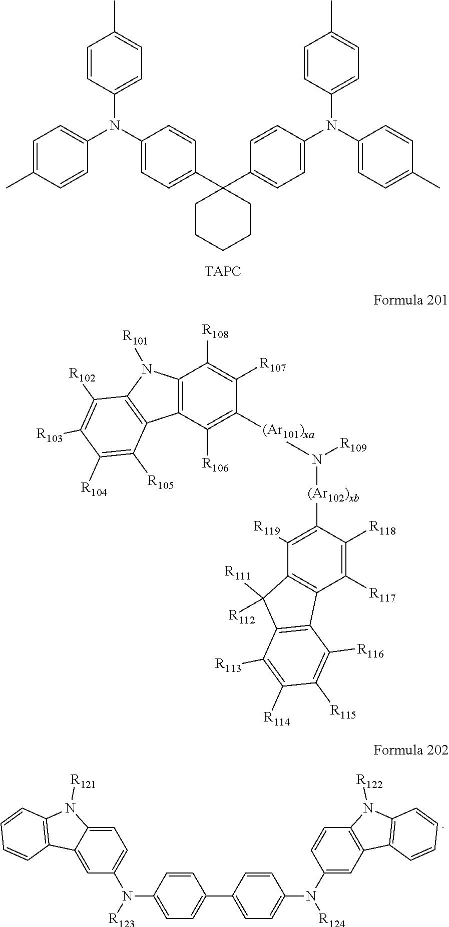

Ar₁₀₁ and Ar₁₀₂ in Formula 201 may each independently be selected from:

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group; and

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

In Formula 201, xa and xb may each independently be an integer from 0 to 5, or 0, 1, or 2. For example, xa is 1 and xb is 0, but xa and xb are not limited thereto.

R₁₀₁ to R₁₀₈, R₁₁₁ to R₁₁₉, and R₁₂₁ to R₁₂₄ in Formulae 201 and 202 may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, and a C₁-C₁₀ alkyl group (for example, a methyl group, an ethyl group, a propyl group, a butyl group, a pentyl group, a hexyl group, and so on), or a C₁-C₁₀ alkoxy group (for example, a methoxy group, an ethoxy group, a propoxy group, a butoxy group, a pentoxy group, and so on);

a C₁-C₁₀ alkyl group or a C₁-C₁₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a phenyl group, a naphthyl group, an anthracenyl group, a fluorenyl group, and a pyrenyl group; and

a phenyl group, a naphthyl group, an anthracenyl group, a fluorenyl group, and a pyrenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group,

but embodiments of the present disclosure are not limited thereto.

R₁₀₉ in Formula 201 may be selected from:

a phenyl group, a naphthyl group, an anthracenyl group, and a pyridinyl group; and

a phenyl group, a naphthyl group, an anthracenyl group, and a pyridinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, and a pyridinyl group.

According to an embodiment, the compound represented by Formula 201 may be represented by Formula 201A, but embodiments of the present disclosure are not limited thereto:

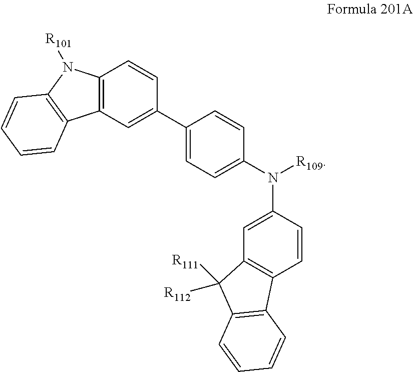

R₁₀₁, R₁₁₁, R₁₁₂, and R₁₀₉ in Formula 201A may be understood by referring to the description provided herein.

For example, the compound represented by Formula 201, and the compound represented by Formula 202 may include compounds HT1 to HT20 illustrated below, but embodiments of the present disclosure are not limited thereto.

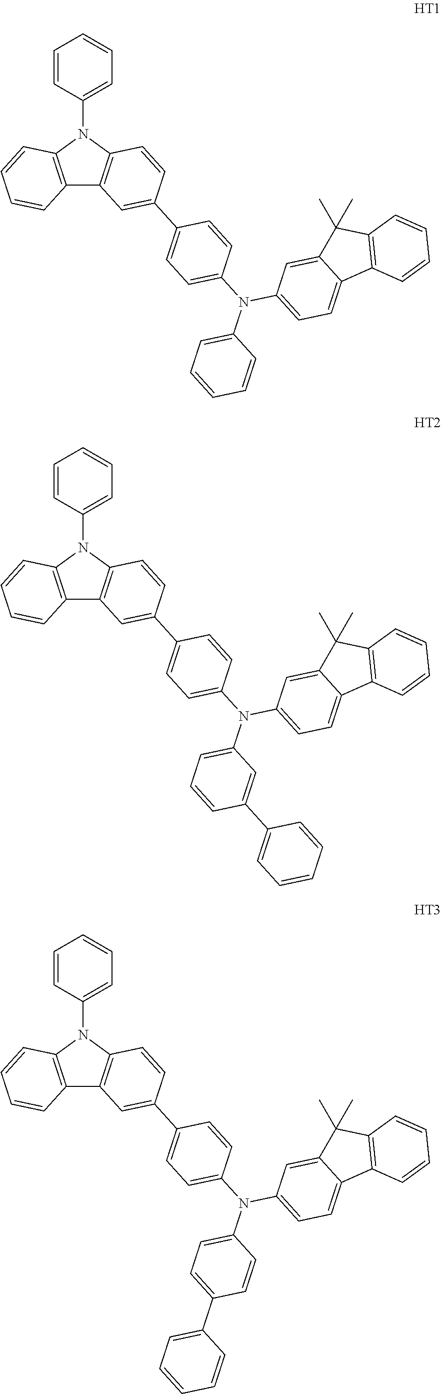 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

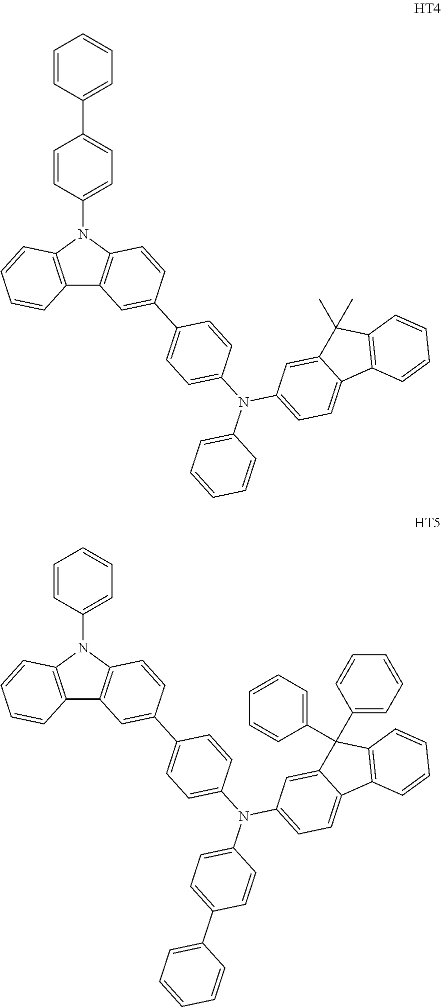 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

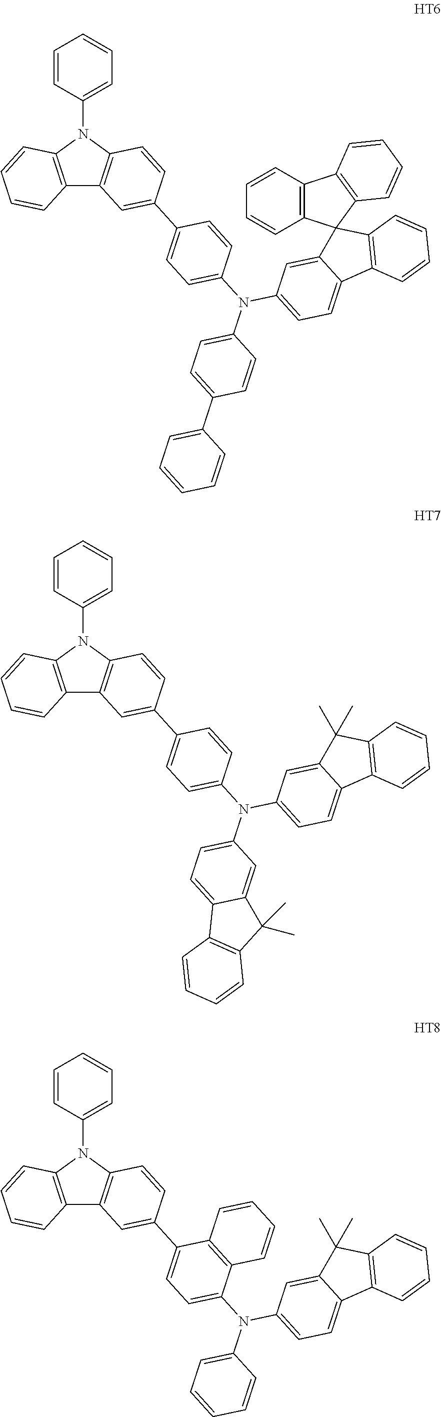 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21]

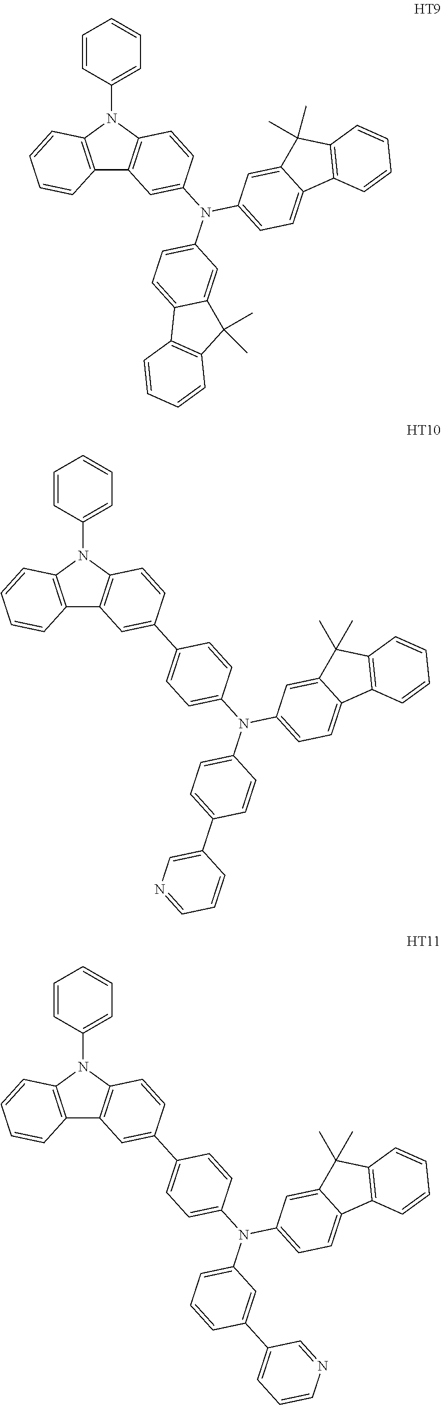 [SMILES: CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21]

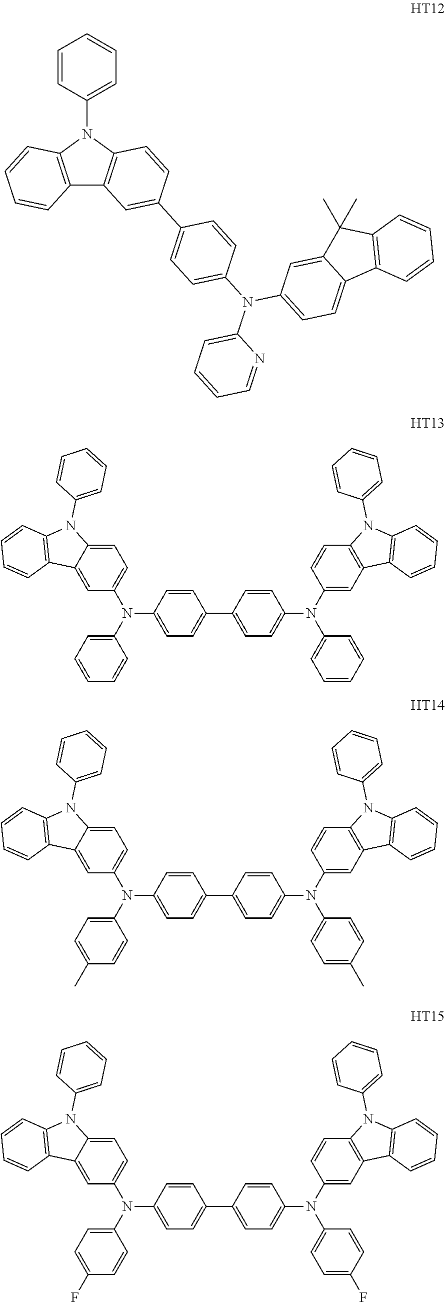 [SMILES: CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

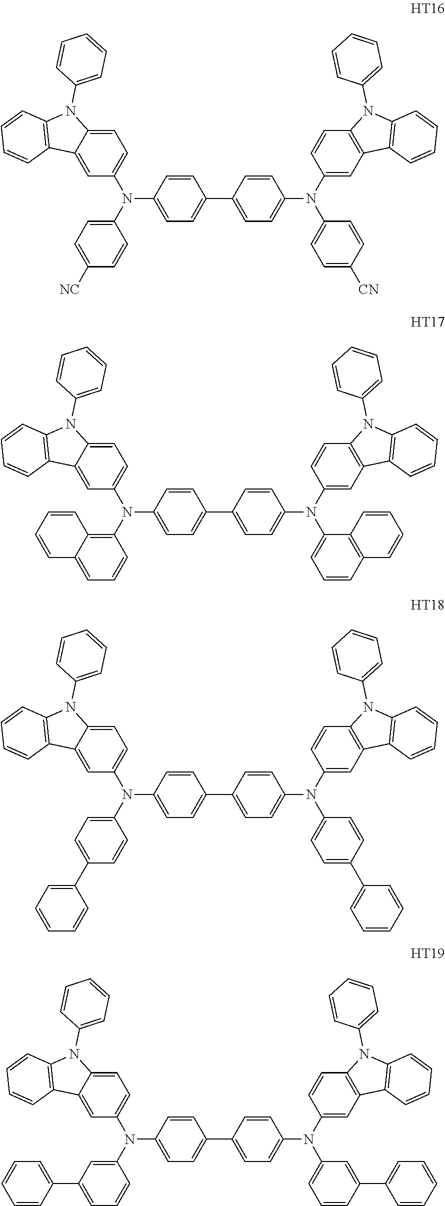 [SMILES: N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

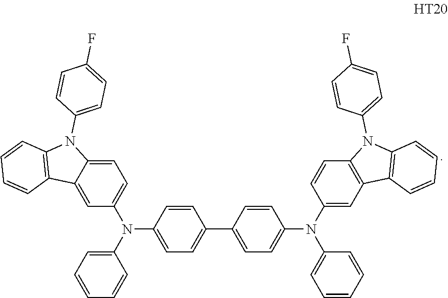 [SMILES: Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Å to about 10,000 Å, for example, about 100 Å to about 1,000 Å. When the hole transport region includes a hole injection layer and a hole transport layer, the thickness of the hole injection layer may be in a range of about 100 Å to about 10,000 Å, and for example, about 100 Å to about 1,000 Å, and the thickness of the hole transport layer may be in a range of about 50 Å to about 2,000 Å, and for example, about 100 Å to about 1,500 Å. While not wishing to be bound by theory, it is understood that when the thicknesses of the hole transport region, the hole injection layer and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one selected from a quinone derivative, a metal oxide, and a cyano group-containing compound, but embodiments of the present disclosure are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenium oxide; and a cyano group-containing compound, such as Compound HT-D1 or HP-1, but embodiments of the present disclosure are not limited thereto:

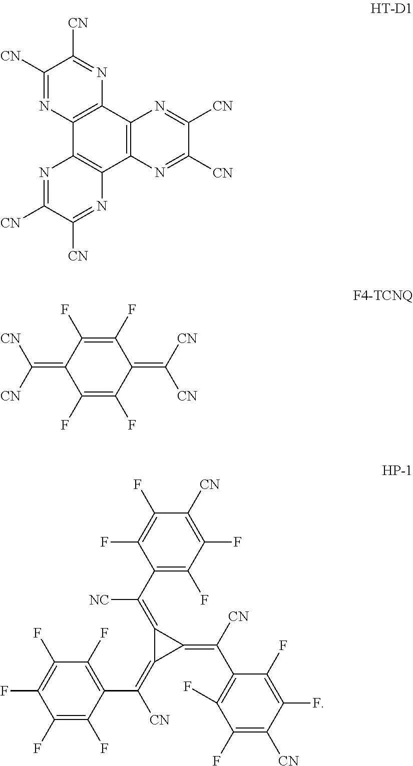

The hole transport region may include a buffer layer.

In one or more embodiments, the buffer layer may compensate for an optical resonance distance according to a wavelength of light emitted from the emission layer, and thus, efficiency of a formed organic light-emitting device may be improved.

The electron transport region may further include an electron blocking layer. The electron blocking layer may include, for example, mCP, but a material therefor is not limited thereto.

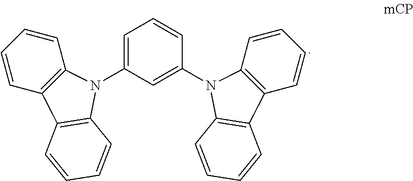 [SMILES: c1cc(-n2c3ccccc3c3ccccc32)cc(-n2c3ccccc3c3ccccc32)c1]

Then, an emission layer (EML) may be formed on the hole transport region by vacuum deposition, spin coating, casting, LB deposition, or the like. When the emission layer is formed by vacuum deposition or spin coating, the deposition or coating conditions may be similar to those applied to form the hole injection layer although the deposition or coating conditions may vary according to the material that is used to form the emission layer.

When the organic light-emitting device is a full-color organic light-emitting device, the emission layer may be patterned into a red emission layer, a green emission layer, and a blue emission layer. In one or more embodiments, due to a stack structure including a red emission layer, a green emission layer, and/or a blue emission layer, the emission layer may emit white light.

The emission layer is the same as described above.

A thickness of the emission layer may be in a range of about 100 Å to about 1,000 Å, for example, about 200 Å to about 600 Å. While not wishing to be bound by theory, it is understood that when the thickness of the emission layer is within this range, excellent light-emission characteristics may be obtained without a substantial increase in driving voltage.

Then, an electron transport region may be disposed on the emission layer.

The electron transport region may include at least one selected from a hole blocking layer, an electron transport layer, and an electron injection layer.

For example, the electron transport region may have a hole blocking layer/electron transport layer/electron injection layer structure or an electron transport layer/electron injection layer structure, but the structure of the electron transport region is not limited thereto. The electron transport layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, at least one of BCP and Bphen, but may also include other materials.

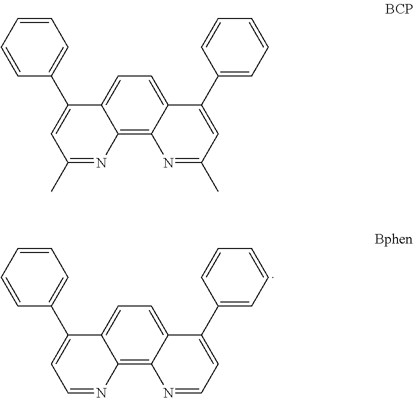 [SMILES: Cc1cc(-c2ccccc2)c2ccc3c(-c4ccccc4)cc(C)nc3c2n1 ; c1ccc(-c2ccnc3c2ccc2c(-c4ccccc4)ccnc23)cc1]

The hole blocking layer may include a compound selected from the hosts described above. In an embodiment, the hole blocking layer may include Compound H19, but embodiments of the present disclosure are not limited thereto.

A thickness of the hole blocking layer may be in a range of about 20 Å to about 1,000 Å, for example, about 30 Å to about 300 Å. While not wishing to be bound by theory, it is understood that when the thickness of the hole blocking layer is within these ranges, the hole blocking layer may have improved hole blocking ability without a substantial increase in driving voltage.

The electron transport layer may further include at least one selected from BCP, Bphen, Alq₃, BAlq, TAZ, and NTAZ.

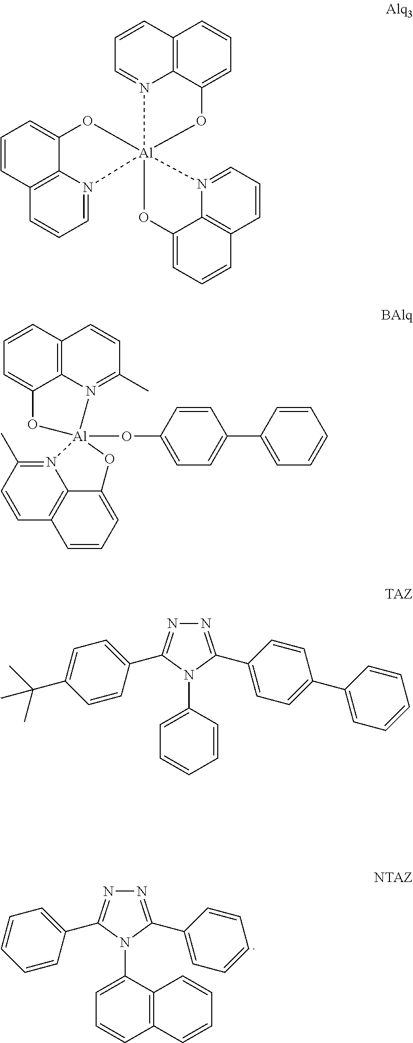

In one or more embodiments, the electron transport layer may include at least one selected from Compounds ET1, ET2, and ET3, but embodiments of the present disclosure are not limited thereto:

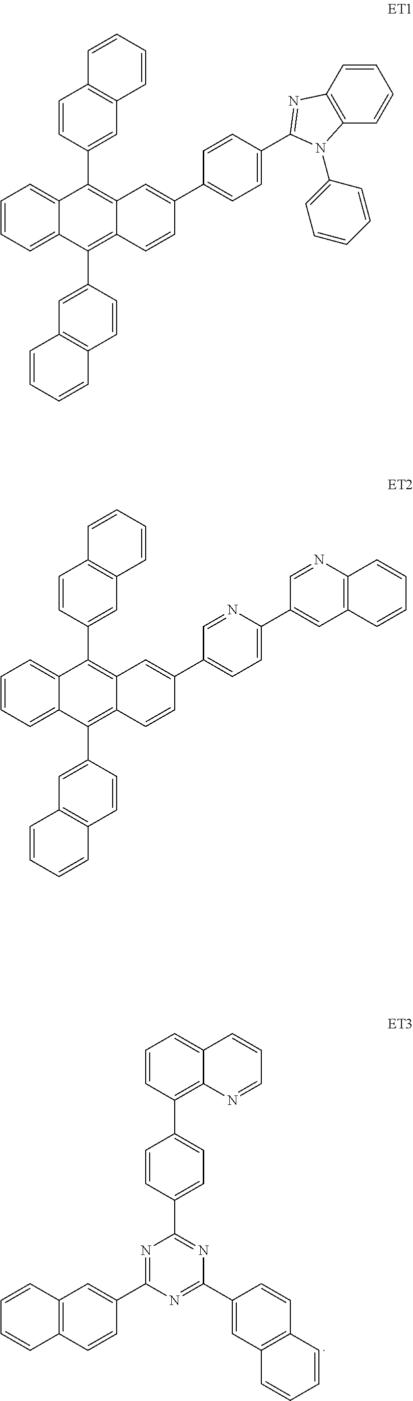 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

A thickness of the electron transport layer may be in a range of about 100 Å to about 1,000 Å, for example, about 150 Å to about 500 Å. While not wishing to be bound by theory, it is understood that when the thickness of the electron transport layer is within the range described above, the electron transport layer may have satisfactory electron transport characteristics without a substantial increase in driving voltage.

Also, the electron transport layer may further include, in addition to the materials described above, a metal-containing material.

The metal-containing material may include a Li complex. The Li complex may include, for example, Compound ET-D1 (LiQ) or ET-D2:

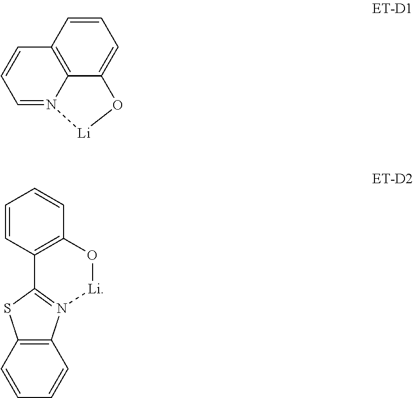

The electron transport region may include an electron injection layer (EIL) that promotes flow of electrons from the second electrode 19 thereinto.

The electron injection layer may include at least one selected from LiF, NaCl, CsF, Li₂O, and BaO.

A thickness of the electron injection layer may be in a range of about 1 Å to about 100 Å, for example, about 3 Å to about 90 Å. While not wishing to be bound by theory, it is understood that when the thickness of the electron injection layer is within the range described above, the electron injection layer may have satisfactory electron injection characteristics without a substantial increase in driving voltage.

The second electrode 19 is disposed on the organic layer 15. The second electrode 19 may be a cathode. A material for forming the second electrode 19 may be selected from metal, an alloy, an electrically conductive compound, and a combination thereof, which have a relatively low work function. For example, lithium (Li), magnesium (Mg), aluminum (Al), aluminum-lithium (Al—Li), calcium (Ca), magnesium-indium (Mg—In), or magnesium-silver (Mg—Ag) may be used as a material for forming the second electrode 19. In one or more embodiments, to manufacture a top-emission type light-emitting device, a transmissive electrode formed using ITO or IZO may be used as the second electrode 19.

Hereinbefore, the organic light-emitting device has been described with reference to FIG. 1, but embodiments of the present disclosure are not limited thereto.

The term “C₁-C₆₀ alkyl group” as used herein refers to a linear or branched saturated aliphatic hydrocarbon monovalent group having 1 to 60 carbon atoms, and non-limiting examples thereof include a methyl group, an ethyl group, a propyl group, an iso-butyl group, a sec-butyl group, a tert-butyl group, a pentyl group, an iso-amyl group, and a hexyl group. The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁ (wherein A₁₀₁ is the C₁-C₆₀ alkyl group), and non-limiting examples thereof include a methoxy group, an ethoxy group, and an iso-propyloxy group.

The term “C₂-C₆₀ alkenyl group” as used herein refers to a hydrocarbon group formed by including at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by including at least one carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms, and non-limiting examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₂-C₁₀ heterocycloalkyl group” as used herein refers to a monovalent saturated monocyclic group having at least one heteroatom selected from N, O, P, Si and S as a ring-forming atom and 2 to 10 carbon atoms, and non-limiting examples thereof include a tetrahydrofuranyl group, and a tetrahydrothiophenyl group. The term “C₂-C₁₀ heterocycloalkylene group” as used herein refers to a divalent group having the same structure as the C₂-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in the ring thereof, and which is not aromatic, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₂-C₁₀ heterocycloalkenyl group” as used herein refers to a monovalent monocyclic group that has at least one heteroatom selected from N, O, P, Si, and S as a ring-forming atom, 2 to 10 carbon atoms, and at least one carbon-carbon double bond in its ring. Examples of the C₂-C₁₀ heterocycloalkenyl group are a 2,3-dihydrofuranyl group, and a 2,3-dihydrothiophenyl group. The term “C₂-C₁₀ heterocycloalkenylene group” as used herein refers to a divalent group having the same structure as the C₂-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₂-C₆₀ heteroaryl group” as used herein refers to a monovalent group having a heterocyclic aromatic system that has at least one heteroatom selected from N, O, P, Si, and S as a ring-forming atom, and 2 to 60 carbon atoms. The term “C₂-C₆₀ heteroarylene group,” as used herein refers to a divalent group having a heterocyclic aromatic system that has at least one heteroatom selected from N, O, P, Si, and S as a ring-forming atom, and 2 to 60 carbon atoms. Non-limiting examples of the C₂-C₆₀ heteroaryl group include a pyridinyl group, a pyrimidinyl group, a pyrazinyl group, a pyridazinyl group, a triazinyl group, a quinolinyl group, and an isoquinolinyl group. When the C₂-C₆₀ heteroaryl group and the C₂-C₆₀ heteroarylene group each include two or more rings, the rings may be fused to each other.

The term “C₆-C₆₀ aryloxy group” as used herein refers to —OA₁₀₂ (wherein A₁₀₂ is the C₆-C₆₀ aryl group), a C₆-C₆₀ arylthio group as used herein indicates —SA₁₀₃ (wherein A₁₀₃ is the C₆-C₆₀ aryl group), and the term “C₇-C₆₀ arylalkyl group” as used herein indicates -A₁₀₄A₁₀₅ (wherein A₁₀₄ is the C₆-C₅₉ aryl group and A₁₀₅ is the C₁-C₅₃ alkyl group).

The term “C₂-C₆₀ heteroaryloxy group” as used herein refers to —OA₁₀₆ (wherein A₁₀₆ is the C₂-C₆₀ heteroaryl group), and the term “C₂-C₆₀ heteroarylthio group” as used herein indicates —SA₁₀₇ (wherein A₁₀₇ is the C₂-C₆₀ heteroaryl group).

The term “C₃-C₆₀ heteroarylalkyl group” as used herein refers to -A₁₀₈A₁₀₉ (A₁₀₉ is a C₂-C₅₉ heteroaryl group, and A₁₀₈ is a C₁-C₅₈ alkylene group).

The term “monovalent non-aromatic condensed polycyclic group” as used herein refers to a monovalent group having two or more rings condensed to each other, only carbon atoms (for example, the number of carbon atoms may be in a range of 8 to 60) as a ring-forming atom, and which is not aromatic in its entire molecular structure. Non-limiting examples of the monovalent non-aromatic condensed polycyclic group include a fluorenyl group. The term “divalent non-aromatic condensed polycyclic group,” as used herein, refers to a divalent group having the same structure as the monovalent non-aromatic condensed polycyclic group.

The term “monovalent non-aromatic condensed heteropolycyclic group” as used herein refers to a monovalent group having two or more rings condensed to each other, a heteroatom selected from N, O, P, Si, and S, other than carbon atoms (for example, the number of carbon atoms may be in a range of 2 to 60), as a ring-forming atom, and which is not aromatic in its entire molecular structure. Non-limiting examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group.

The term “C₅-C₆₀ carbocyclic group” as used herein refers to a saturated or unsaturated cyclic group having, as a ring-forming atom, 5 to 60 carbon atoms only. The C₅-C₆₀ carbocyclic group may be a monocyclic group or a polycyclic group, and according to a chemical structure, the C₅-C₆₀ carbocyclic group may be a monovalent group, a divalent group, a trivalent group, a tetravalent group, a pentavalent group, or a hexavalent group.

The term “C₂-C₆₀ heterocyclic group” as used herein refers to a saturated or unsaturated cyclic group having, as a ring-forming atom, at least one heteroatom selected from N, O, Si, P, and S other than 2 to 60 carbon atoms. The C₂-C₆₀ heterocyclic group may be a monocyclic group or a poly cyclic group, and, according to the formula structure, the C₂-C₆₀ heterocyclic group may be a monovalent group, a divalent group, a trivalent group, a tetravalent group, a pentavalent group, or a hexavalent group.

At least one substituent of the substituted C₅-C₆₀ carbocyclic group, the substituted C₂-C₆₀ heterocyclic group, the substituted π electron-depleted nitrogen-containing C₂-C₆₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₇-C₆₀ arylalkyl group, the substituted C₁-C₆₀ heteroaryl group, the substituted C₂-C₆₀ heteroaryloxy group, the substituted C₂-C₆₀ heteroarylthio group, the substituted C₃-C₆₀ heteroarylalkyl group, the substituted monovalent non-aromatic condensed polycyclic group, and the substituted monovalent non-aromatic condensed heteropolycyclic group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉);

—N(Q₃₁)(Q₃₂), —Si(Q₃₃)(Q₃₄)(Q₃₅), —B(Q₃₆)(Q₃₇) and —P(═O)(Q₃₈)(Q₃₉); and

Q₁₁ to Q₁₉, Q₂₁ to Q₂₉, and Q₃₁ to Q₃₉ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one selected from a C₁-C₆₀ alkyl group and a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group.

When a group containing a specified number of carbon atoms is substituted with any of the groups listed in the preceding paragraph, the number of carbon atoms in the resulting “substituted” group is defined as the sum of the carbon atoms contained in the original (unsubstituted) group and the carbon atoms (if any) contained in the substituent. For example, when the term “substituted C1-C30 alkyl” refers to a C1-C30 alkyl group substituted with C6-C30 aryl group, the total number of carbon atoms in the resulting aryl substituted alkyl group is C7-C60.

The term “room temperature” as used herein refers to about 25° C.

Hereinafter, a compound and an organic light-emitting device according to embodiments are described in detail with reference to Synthesis Examples and Examples. However, the organic light-emitting device is not limited thereto. The wording “B was used instead of A” used in describing Synthesis Examples means that an amount of A used was identical to an amount of B used, in terms of a molar equivalent.

### EXAMPLES

### Synthesis Example 1: Synthesis of Intermediates 1a and 1b

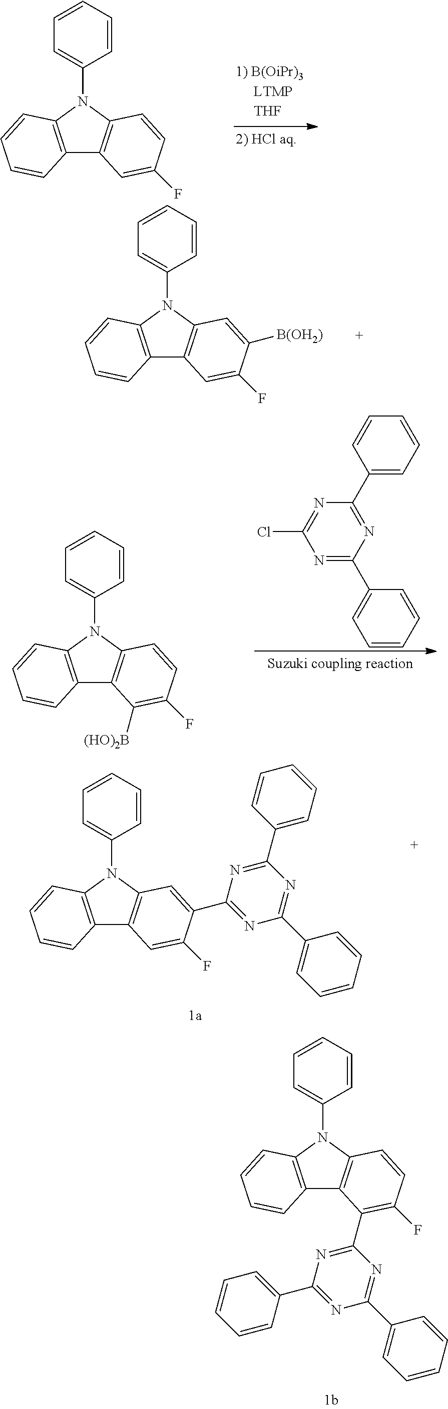

10.45 grams (g) (40 millimoles, mmol) of 3-fluoro-N-phenylcarbazole, 11.28 g (60 mmol) of tri(isopropyl)borate, and 200 milliliters (ml) of tetrahydrofuran (THF) were added to a three-neck flask and cooled to a temperature of −75° C. in a nitrogen atmosphere. Then, a lithium tetramethylpiperidide (LTMP) solution prepared by using 8.48 g (60 mmol) of 2,2,6,6-tetramethylpiperidine, 60 ml of THF, and 20.8 ml (52 mmol) of n-BuLi hexane solution was added thereto, stirred at a temperature of −60° C. for 2 hours, and further stirred at room temperature for 12 hours. After the reaction was completed, 100 ml of 1 N (one normal) HCl (aq) (aq=aqueous) was added thereto, and the mixture was stirred at room temperature for 2 hours. A resultant extracted by using AcOEt was dried by using MgSO₄. Then, the resultant was filtered, concentrated, and immediately added to a three-neck flask.

Then, 8.58 g (32.06 mmol) of 2-chloro-4,6-diphenyl-1,3,5-triazine, 6.65 g (48.09 mmol) of potassium carbonate, 48 ml of water, 320 ml of THF, 216 milligrams (mg) (0.96 mmol) of Pd(OAc)₂, and 584 mg (1.92 mmol) of tri-o-tolylphosphine were added to the three-neck flask and refluxed for 6 hours in a nitrogen atmosphere.

After the reaction was completed, a solid A precipitated by cooling to room temperature was filtered and collected, and a filtrate B was collected and extracted by using CH₂Cl₂, dried by using MgSO₄, and filtered and concentrated by using a silica gel pad. Then, a recrystallization thereof was performed by using AcOEt, thereby obtaining Intermediate 1b (yield: 7%). On the other hand, the collected solid A was dispersed and washed in AcOEt and then dried, thereby obtaining Intermediate 1a (yield: 36%).

### Synthesis Example 2: Synthesis of Compound 1

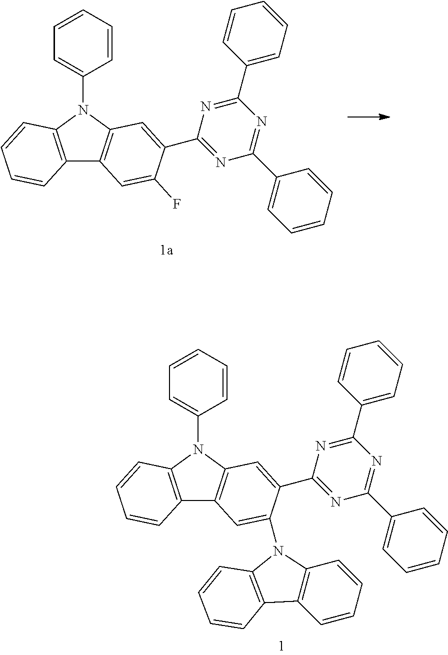 [SMILES: Fc1cc2c3ccccc3n(-c3ccccc3)c2cc1-c1nc(-c2ccccc2)nc(-c2ccccc2)n1 ; c1ccc(-c2nc(-c3ccccc3)nc(-c3cc4c(cc3-n3c5ccccc5c5ccccc53)c3ccccc3n4-c3ccccc3)n2)cc1]

2.98 g (6.05 mmol) of Intermediate 1a, 6.07 g (36.3 mmol) of carbazole, 3.4 g (30.3 mmol) of tert-BuOK, and 18 ml of dimethylformamide (DMF) were added to a three-neck flask, and the mixture was stirred at a temperature of 170° C. for 18 hours in a nitrogen atmosphere. After the reaction was completed, the mixture was cooled to room temperature, and a solid precipitated by adding MeOH was filtered and collected. Then, the solid was vacuum-dried (60° C., 4 hours) and purified by column chromatography (hexane:CH₂Cl₂=1:1). Then, the solid was dispersed and washed in AcOEt, thereby obtaining 13.65 g (yield: 49%) of Compound 1.

1H-NMR (300 MHz, CD₂Cl₂): 8.6 (1H), 8.45 (1H), 8.2 (1H), 8.08 (2H), 8.0-7.9 (4H), 7.85-7.65 (4H), 7.65-7.52 (3H), 7.52-7.15 (13H).

MALDI-MS Calcd: 639.24, Found: 639.26.

### Synthesis Example 3: Synthesis of Compound 2

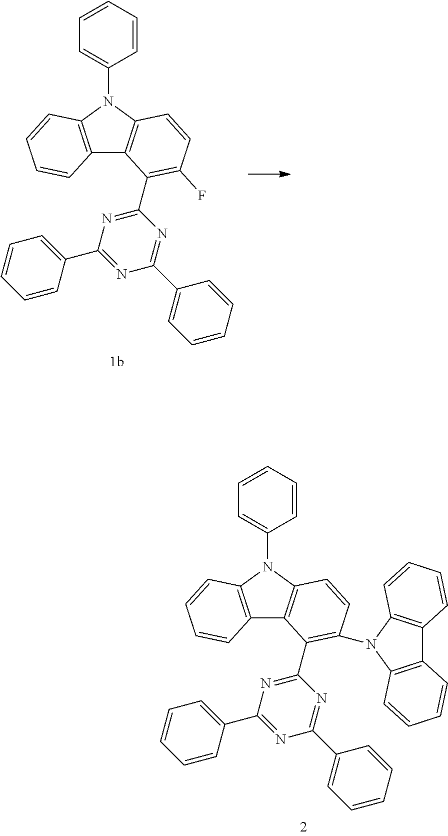 [SMILES: Fc1ccc2c(c1-c1nc(-c3ccccc3)nc(-c3ccccc3)n1)c1ccccc1n2-c1ccccc1 ; c1ccc(-c2nc(-c3ccccc3)nc(-c3c(-n4c5ccccc5c5ccccc54)ccc4c3c3ccccc3n4-c3ccccc3)n2)cc1]

Compound 2 (yield: 85%) was synthesized in the same manner as in Synthesis Example 2, except that Intermediate 1b was used instead of Intermediate 1a.

1H-NMR (300 MHz, CD₂Cl₂): 8.08 (4H), 7.90 (2H), 7.8-7.55 (8H), 7.55-7.25 (12H), 7.13 (2H), 7.01 (1H).

MALDI-MS Calcd: 639.24, Found: 639.26.

### Synthesis Example 4: Synthesis of Compound 3

Synthesis of Intermediate 3a

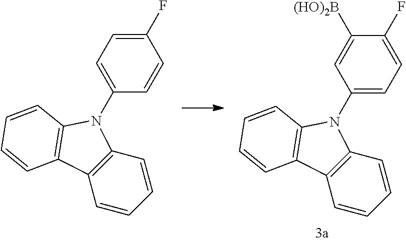 [SMILES: Fc1ccc(-n2c3ccccc3c3ccccc32)cc1 ; OB(O)c1cc(-n2c3ccccc3c3ccccc32)ccc1F]

47.28 g (180.9 mmol) of 9-(4-fluorophenyl)-9H-carbazole, 200 mmol of tri(isopropyl)borate, and 724 ml of THF, and 33.22 g (235.2 mmol) of 2,2,6,6-tetramethylpiperidine were added to a three-neck flask and cooled to a temperature of −70° C. in a nitrogen atmosphere. Then, 86.8 ml (217.1 mmol) of n-BuLi (2.5 M (molar) in hexane) was added dropwise thereto. Then, the mixture was stirred at a temperature of −40° C. for 1 hour and additionally stirred at room temperature for 3 hours. After the reaction was completed, the mixture was concentrated until the resultant halved in volume. Then, 1 N HCl (aq) was added thereto to adjust pH to 1, and the resultant was stirred at room temperature for 2 hours. Then, a product extracted by using ethyl acetate was washed by using water, dried by using MgSO₄, filtered, concentrated, and then vacuum-dried at a temperature of 60° C. for 12 hours. The result obtained therefrom was dissolved in 200 ml of toluene, and 500 ml of hexane was added thereto. The mixture was refluxed for 6 hours. After cooling to room temperature, a precipitated solid was filtered, collected, and then vacuum-dried at a temperature of 60° C. for 12 hours, thereby obtaining 47.3 g (yield: 86%) of Intermediate 3a.

Synthesis of Intermediate 3b

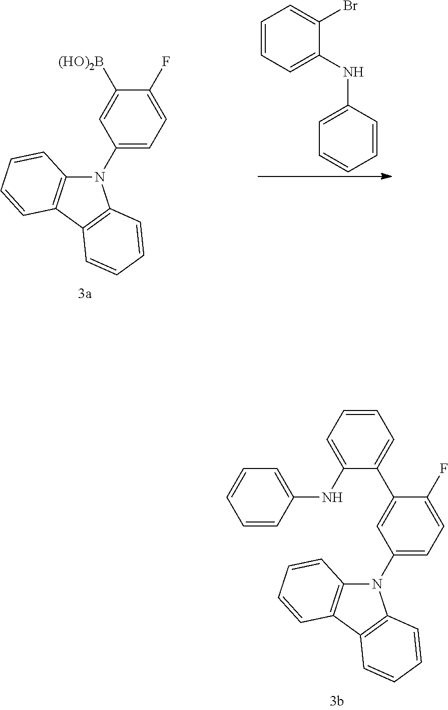 [SMILES: OB(O)c1cc(-n2c3ccccc3c3ccccc32)ccc1F ; Brc1ccccc1Nc1ccccc1 ; Fc1ccc(-n2c3ccccc3c3ccccc32)cc1-c1ccccc1Nc1ccccc1]

16.78 g (55 mmol) of Intermediate 3a, 12.41 g (50 mmol) of 2-bromo-N-phenylaniline, 100 ml of THF, 10.37 g (75 mmol) of potassium carbonate, and 75 ml of water were added to a three-neck flask and uniformly mixed in a nitrogen atmosphere.

Then, 561 mg (2.5 mmol) of palladium acetate and 1.52 g (5 mmol) of tri-o-tolylphosphine were added thereto, and the mixture was stirred at a temperature of 80° C. for 2 hours.

Then, after cooling to room temperature, a solid was extracted by using toluene, dried by using MgSO₄, filtered and concentrated by using a silica gel pad, then purified by silica gel chromatography (hexane:CH₂Cl₂=6:4), thereby obtaining 18.6 g (yield: 87%) of Intermediate 3b.

Synthesis of Intermediate 3c

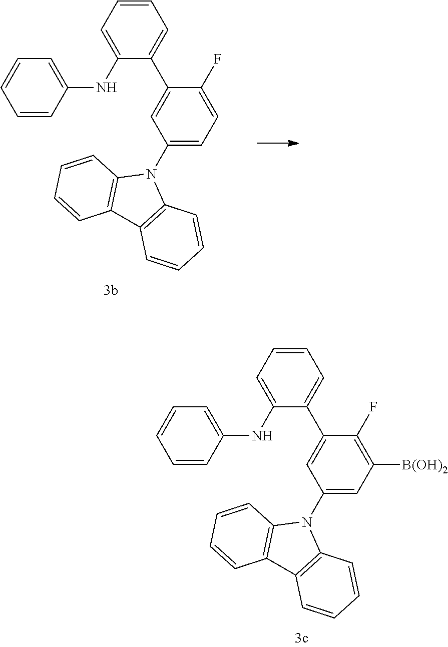 [SMILES: Fc1ccc(-n2c3ccccc3c3ccccc32)cc1-c1ccccc1Nc1ccccc1 ; OB(O)c1cc(-n2c3ccccc3c3ccccc32)cc(-c2ccccc2Nc2ccccc2)c1F]

18.6 g (43.4 mmol) of Intermediate 3b, 217 ml of THF, 60 mmol of tri(isopropyl)borate, and 9.2 g (65.1 mmol) of 2,2,6,6-tetramethylpiperidine were added to a three-neck flask and cooled to a temperature of −70° C. in a nitrogen atmosphere, and 38.2 ml (95.5 mmol) of n-BuLi (2.5M in hexane) was added thereto dropwise. Then, the mixture was stirred at a temperature of −40° C. for 1 hour and additionally stirred at room temperature for 12 hours. After the reaction was completed, the mixture was concentrated until the resultant halved in volume. Then, 1 N HCl (aq) was added thereto to adjust pH to 1, and the resultant was stirred at room temperature for 2 hours. Then, a product extracted by using ethyl acetate was washed by using water, dried by using MgSO₄, filtered, concentrated, and then purified by silica gel chromatography (CH₂Cl₂:hexane=7:3, CH₂Cl₂:MeOH=9:1, gradation), thereby obtaining 15.9 g (yield: 77%) of Intermediate 3c.

Synthesis of Intermediate 3d

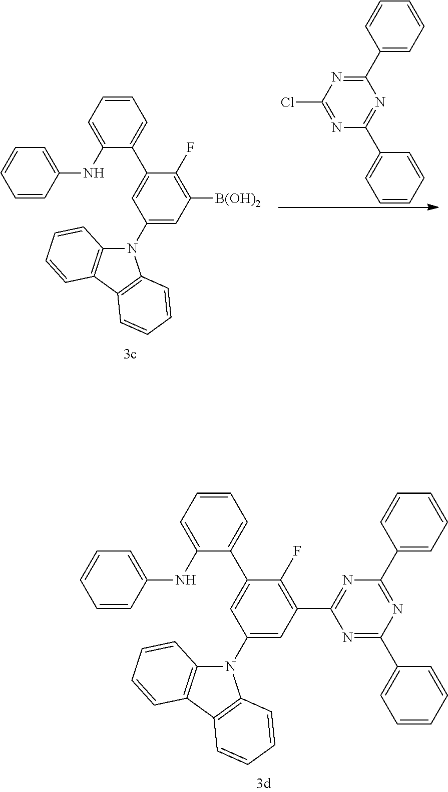 [SMILES: OB(O)c1cc(-n2c3ccccc3c3ccccc32)cc(-c2ccccc2Nc2ccccc2)c1F ; Clc1nc(-c2ccccc2)nc(-c2ccccc2)n1 ; Fc1c(-c2nc(-c3ccccc3)nc(-c3ccccc3)n2)cc(-n2c3ccccc3c3ccccc32)cc1-c1ccccc1Nc1ccccc1]

15.9 g (33.6 mmol) of Intermediate 3c, 8.03 g (30 mmol) of 2-chloro-4,6-diphenyl-1,3,5-triazine, 150 ml of THF, 6.22 g (45 mmol) of potassium carbonate, and 45 ml of water were added to a three-neck flask and uniformly mixed in a nitrogen atmosphere.

Then, 202 mg (0.9 mmol) of palladium acetate and 548 mg (1.8 mmol) of tri-o-tolylphosphine were added thereto, and the mixture was stirred at a temperature of 70° C. for 2 hours.

After cooling to room temperature, a solid was extracted by using toluene, dried by using MgSO₄, filtered and concentrated by using a silica gel pad, and then purified by silica gel chromatography (hexane:CH₂Cl₂=6:4), thereby obtaining 18.3 g (yield: 93%) of Intermediate 3d.

Synthesis of Compound 3

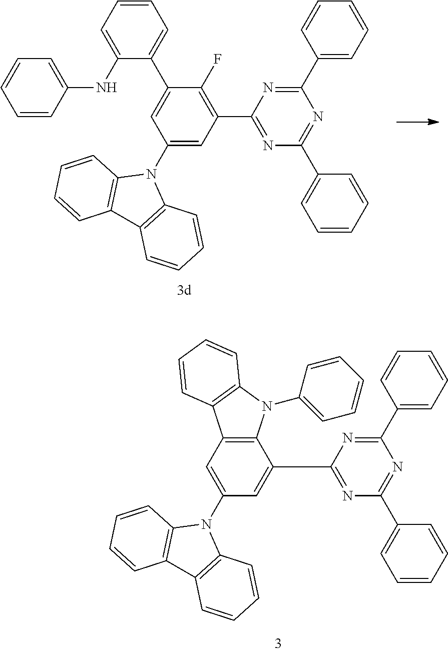 [SMILES: Fc1c(-c2nc(-c3ccccc3)nc(-c3ccccc3)n2)cc(-n2c3ccccc3c3ccccc32)cc1-c1ccccc1Nc1ccccc1 ; c1ccc(-c2nc(-c3ccccc3)nc(-c3cc(-n4c5ccccc5c5ccccc54)cc4c5ccccc5n(-c5ccccc5)c34)n2)cc1]

18.3 g (27.8 mmol) of Intermediate 3d and 278 ml of xylene were added to a three-neck flask and uniformly mixed in a nitrogen atmosphere, and 4.68 g (41.7 mmol) of tert-BuOK was added thereto, and the mixture was stirred at a temperature of 150° C. for 6 hours.

Then, after cooling to room temperature, a solid was filtered by using celite, additionally filtered and concentrated by using a silica gel pad, and then purified by silica gel chromatography (hexane:toluene=1:1), thereby obtaining 15.8 g (yield: 89%) of Compound 3.

1H-NMR (300 MHz, CD₂Cl₂): 8.6-8.4 (5H), 8.3-8.1 (4H), 7.65-7.25 (17H), 7.0-6.8 (3H).

MALDI-MS Calcd: 639.24, Found: 639.26.

### Synthesis Example 5: Synthesis of Compound 4

Synthesis of Intermediate 4a

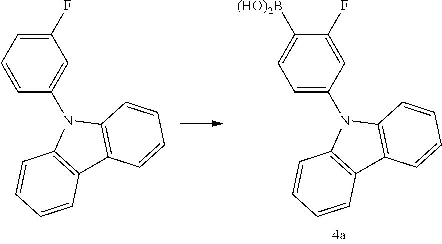 [SMILES: Fc1cccc(-n2c3ccccc3c3ccccc32)c1 ; OB(O)c1ccc(-n2c3ccccc3c3ccccc32)cc1F]

Intermediate 4a was synthesized in the same manner as Intermediate 3a of Synthesis Example 4, except that 9-(3-fluorophenyl)-9H-carbazole was used instead of 9-(4-fluorophenyl)-9H-carbazole.

Synthesis of Intermediate 4b

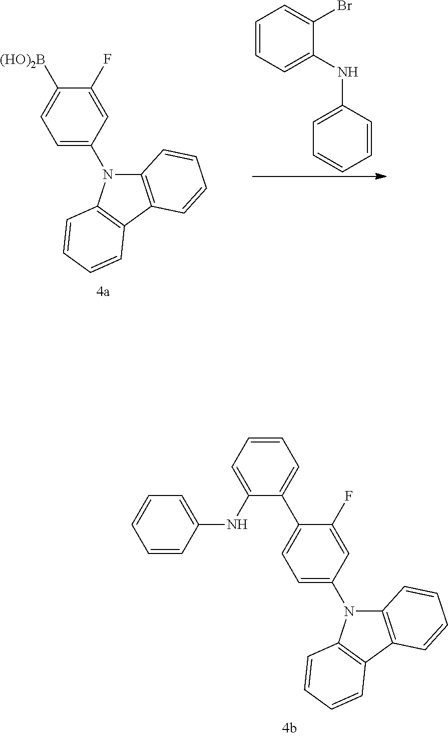 [SMILES: OB(O)c1ccc(-n2c3ccccc3c3ccccc32)cc1F ; Brc1ccccc1Nc1ccccc1 ; Fc1cc(-n2c3ccccc3c3ccccc32)ccc1-c1ccccc1Nc1ccccc1]

Intermediate 4b was synthesized in the same manner as Intermediate 3b of Synthesis Example 4, except that Intermediate 4a was used instead of Intermediate 3a.

Synthesis of Intermediate 4c

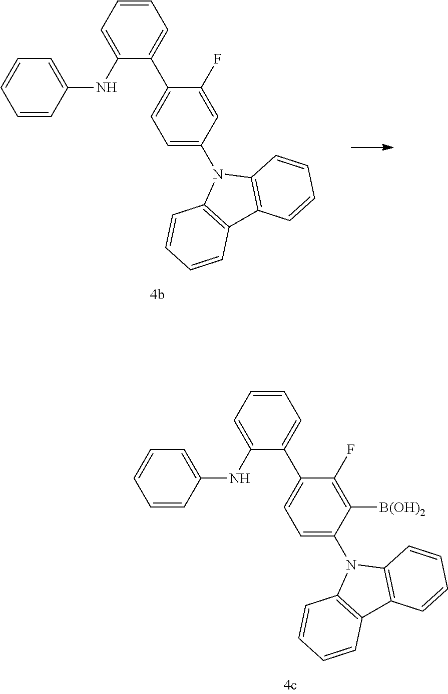 [SMILES: Fc1cc(-n2c3ccccc3c3ccccc32)ccc1-c1ccccc1Nc1ccccc1 ; OB(O)c1c(-n2c3ccccc3c3ccccc32)ccc(-c2ccccc2Nc2ccccc2)c1F]

Intermediate 4c was synthesized in the same manner as Intermediate 3c of Synthesis Example 4, except that Intermediate 4b was used instead of Intermediate 3b.

Synthesis of Intermediate 4d

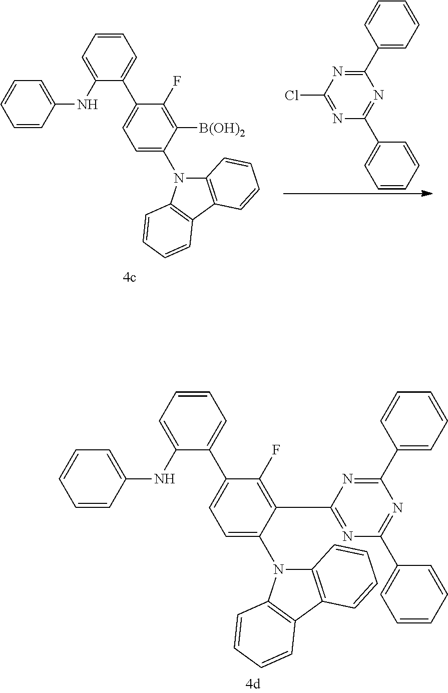 [SMILES: OB(O)c1c(-n2c3ccccc3c3ccccc32)ccc(-c2ccccc2Nc2ccccc2)c1F ; Clc1nc(-c2ccccc2)nc(-c2ccccc2)n1 ; Fc1c(-c2ccccc2Nc2ccccc2)ccc(-n2c3ccccc3c3ccccc32)c1-c1nc(-c2ccccc2)nc(-c2ccccc2)n1]

Intermediate 4d was synthesized in the same manner as Intermediate 3d of Synthesis Example 4, except that Intermediate 4c was used instead of Intermediate 3c.

Synthesis of Compound 4

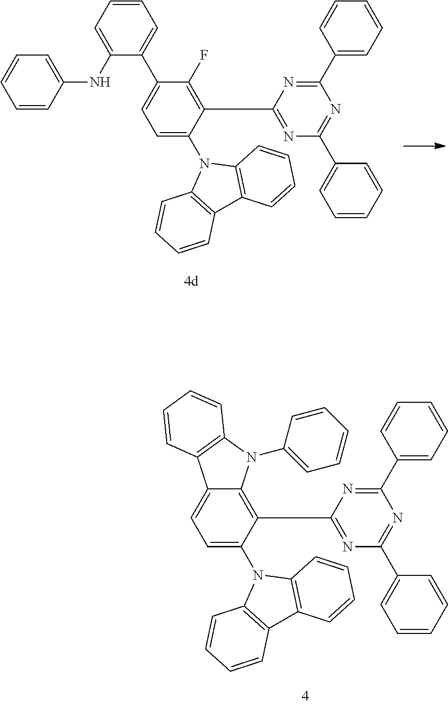 [SMILES: Fc1c(-c2ccccc2Nc2ccccc2)ccc(-n2c3ccccc3c3ccccc32)c1-c1nc(-c2ccccc2)nc(-c2ccccc2)n1 ; c1ccc(-c2nc(-c3ccccc3)nc(-c3c(-n4c5ccccc5c5ccccc54)ccc4c5ccccc5n(-c5ccccc5)c34)n2)cc1]

0.34 g (yield: 48%) of Compound 4 was synthesized in the same manner as Compound 3 of Synthesis Example 4, except that Intermediate 4d was used instead of Intermediate 3d.

1H-NMR (300 MHz, CD₂Cl₂): 8.6-8.4 (5H), 8.3-8.1 (4H), 7.65-7.25 (17H), 7.0-6.8 (3H)

MALDI-MS Calcd: 639.24, Found: 640.2.

### Synthesis Example 6: Synthesis of Compound 50

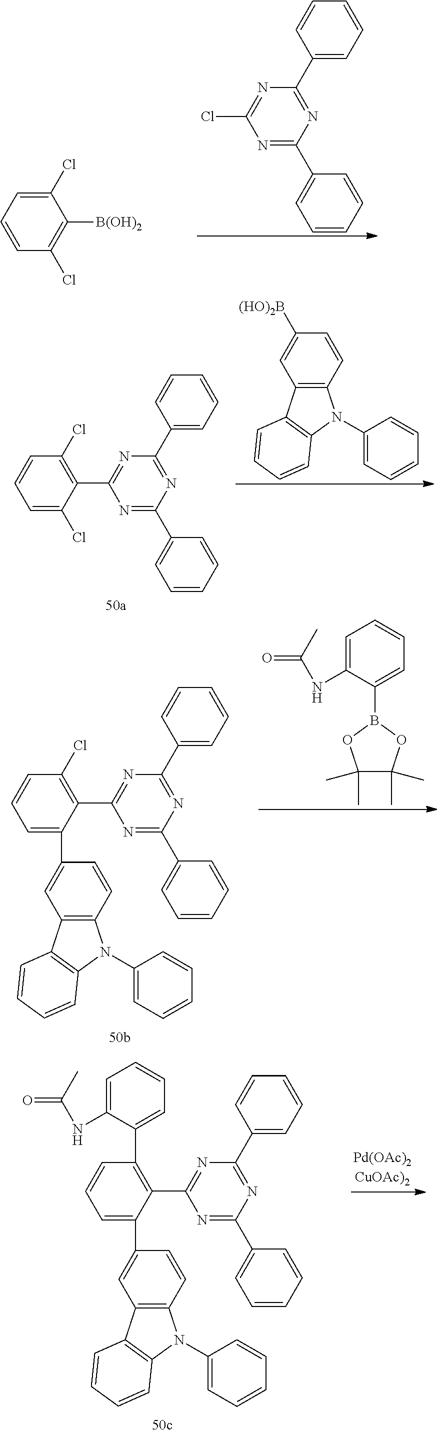

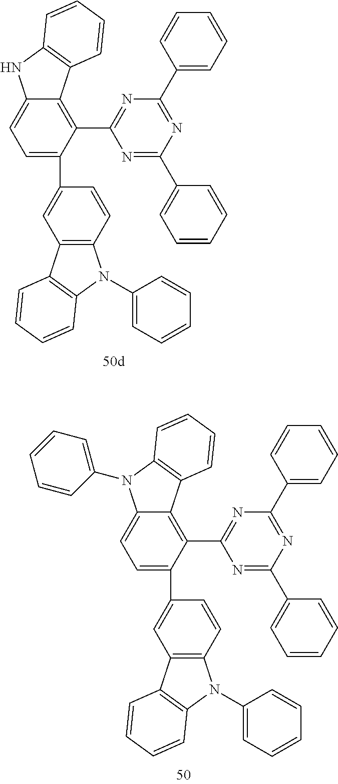 [SMILES: c1ccc(-c2nc(-c3ccccc3)nc(-c3c(-c4ccc5c(c4)c4ccccc4n5-c4ccccc4)ccc4[nH]c5ccccc5c34)n2)cc1 ; c1ccc(-c2nc(-c3ccccc3)nc(-c3c(-c4ccc5c(c4)c4ccccc4n5-c4ccccc4)ccc4c3c3ccccc3n4-c3ccccc3)n2)cc1]

Synthesis of Intermediate 50a

37.35 mmol of 2,6-dichlorophenylboronic acid, 10 g (37.35 mmol) of 2-chloro-4,6-diphenyl-1,3,5-triazine, 10.33 g (74.70 mmol) of 2-chloro-4,6-diphenyl-1,3,5-triazine, 90 ml of water, 90 ml of THF, and 2.158 g (1.87 mmol) of Pd(PPh₃)₄ were added to a three-neck flask and refluxed for 12 hours in a nitrogen atmosphere.

After the reaction was completed, a solid precipitated by cooling to room temperature was filtered and collected, and a filtrate was extracted by using CH₂Cl₂, dried by using MgSO₄, and filtered and concentrated by using a silica gel pad. Then, a recrystallization thereof was performed by using CH₂Cl₂:Hexane, thereby obtaining Intermediate 50a (yield: 96%).

Synthesis of Intermediate 50b

Intermediate 50b was synthesized in the same manner as Intermediate 50a, except that 9-phenyl-9H-carbazol-3-yl-3-boronic acid and Intermediate 50a were used instead of 2,6-dichlorophenylboronic acid and of 2-chloro-4,6-diphenyl-1,3,5-triazine, respectively.

Synthesis of Intermediate 50c

Intermediate 50c was synthesized in the same manner as Intermediate 50a, except that N-(2-(4,4,5,5-tetramethyl-1,3,2-dioxaborolan-2-yl)phenyl)acetamide and Intermediate 50b were used instead of 2,6-dichlorophenylboronic acid and 2-chloro-4,6-diphenyl-1,3,5-triazine, respectively.

Synthesis of Intermediate 50d

9.8 g (27.8 mmol) of Intermediate 50c and 78 ml of xylene were added to a three-neck flask and uniformly mixed in a nitrogen atmosphere, and Pd(OAc)₂ (0.02 M) and Cu(OAc)₂ (0.02 M) were added thereto, and the mixture was stirred at a temperature of 150° C. for 6 hours.

Then, after cooling to room temperature, a solid was filtered by using celite, additionally filtered and concentrated by using a silica gel pad, and then purified by silica gel chromatography (hexane:CH₂Cl₂=1:1), thereby obtaining 5.6 g (yield: 61%) of Intermediate 50d.

Synthesis of Compound 50

9.64 mmol of Intermediate 50d, 1.513 g (9.64 mmol) of bromobenzene, 1.684 g (17.52 mmol) of tert-sodium butoxide, 0.321 g (0.35 mmol) of Pd₂(dba)₃, and 0.284 g (1.4 mmol) of tri-tert-butyl phosphine were mixed with 45 mL of toluene for a reaction at a temperature of 100° C. for 12 hours. After the reaction was completed, the mixture was cooled to room temperature, and a solid compound precipitated by methanol was filtered. Then, a resultant was purified by column chromatography and vacuum-dried, thereby obtaining 5.8 g (92%) of Compound 50.

1H-NMR (300 MHz, CD₂Cl₂): 8.5-8.6 (2H), 8.1-8.4 (4H), 7.5-8.0 (23H), 7.3-7.4 (2H), 7.0-7.1 (2H)

MALDI-MS Calcd: 715.27, Found: 716.2.

### Synthesis Example 7: Synthesis of Compound 669

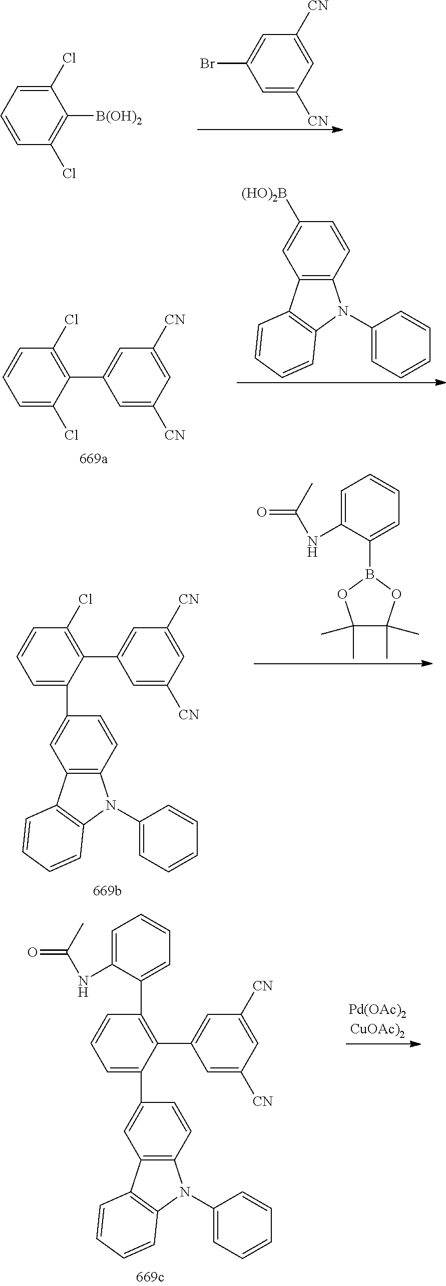

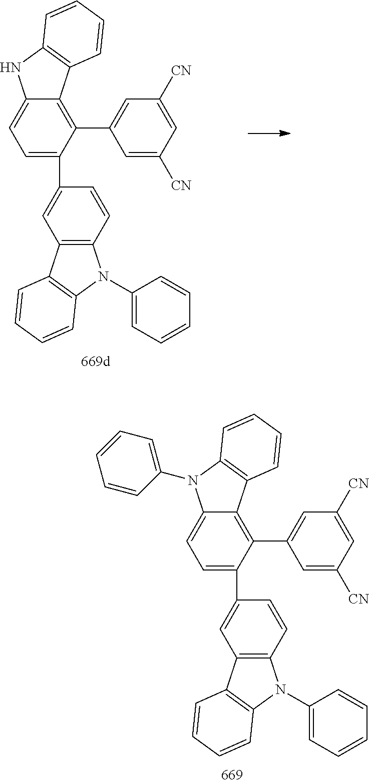 [SMILES: N#Cc1cc(C#N)cc(-c2c(-c3ccc4c(c3)c3ccccc3n4-c3ccccc3)ccc3[nH]c4ccccc4c23)c1 ; N#Cc1cc(C#N)cc(-c2c(-c3ccc4c(c3)c3ccccc3n4-c3ccccc3)ccc3c2c2ccccc2n3-c2ccccc2)c1]

0.5 g (yield: 72%) of Compound 669 was obtained in the same manner as in the Synthesis Example 6, except that 5-bromobenzene-1,3-dinitrile was used instead of 2-chloro-4,6-diphenyl-1,3,5-triazine in the synthesis of Intermediate 50a.

1H-NMR (300 MHz, CD₂Cl₂): 8.5 (2H), 8.1-8.2 (3H), 7.5-8.0 (20H), 7.3-7.4 (2H), 7.1-7.2 (2H)

MALDI-MS Calcd: 610.22, Found: 610.25.

### Synthesis Example 8: Synthesis of Compound 671

Synthesis of Intermediate 671 b

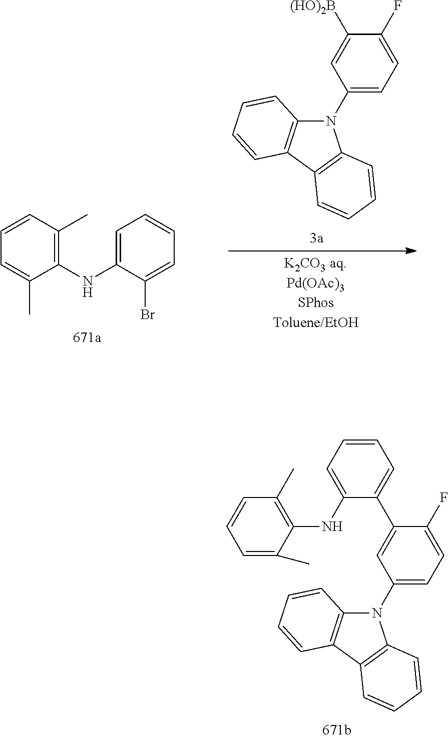 [SMILES: Cc1cccc(C)c1Nc1ccccc1Br ; OB(O)c1cc(-n2c3ccccc3c3ccccc32)ccc1F ; Cc1cccc(C)c1Nc1ccccc1-c1cc(-n2c3ccccc3c3ccccc32)ccc1F]

8.31 g (30.1 mmol) of a starting material 671a, 8.36 g (27.4 mmol) of Intermediate 3a, 37.57 g (54.8 mmol) of K₂CO, 55 ml of water, 55 ml of toluene, 55 ml of ethanol, 2,184 mg (0.82 mmol) of Pd(OAc), 899 mg (2.19 mmol) of 2-dicyclohexylphosphino-2′,6′-dimethoxybiphenyl(S-Phos) were added to a three-neck flask, and the mixture was stirred at a temperature of 80° C. for 5 hours in a nitrogen atmosphere. 200 ml of toluene was added to the mixture obtained therefrom, and a resultant was filtered by using Celite, washed twice by using water, dried by using MgSO₄, filtered and concentrated by using a silica gel pad, and then, purified by silica gel chromatography (hexane:CH₂Cl₂=7:3), thereby obtaining 6.71 g (14.7 mmol) of Intermediate 671 b (yield: 54%).

Synthesis of Intermediate 671c

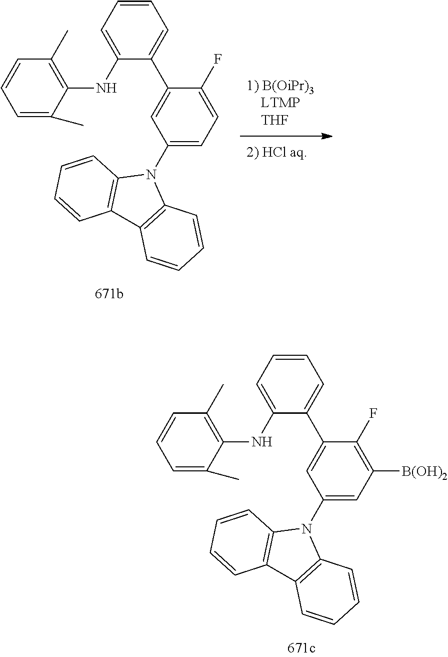 [SMILES: Cc1cccc(C)c1Nc1ccccc1-c1cc(-n2c3ccccc3c3ccccc32)ccc1F ; Cc1cccc(C)c1Nc1ccccc1-c1cc(-n2c3ccccc3c3ccccc32)cc(B(O)O)c1F]

6.71 g (14.7 mmol) of Intermediate 671b, 8.29 g (44.1 mmol) of triisopropyl borate, and 74 ml of THF were added to a three-neck flask. A resultant was subjected to nitrogen substitution, and then, cooled to a temperature of −60° C. Subsequently, lithium 2,2,6,6-tetramethylpiperidide solution prepared by using 6.85 g (48.5 mmol) of 2,2,6,6-tetramethylpiperidine, 17.6 ml (44.1 mmol) of n-butyllithium (2.5 M), and 45 ml of THF was added thereto for 10 minutes, stirred at a temperature of −60° C. for 2 hours, and then, stirred at room temperature for 2 hours. A small amount of methanol was added to a resultant, and a small amount of water and 100 ml of 1 N hydrochloric acid aqueous solution were sequentially added thereto, stirred at room temperature for 2 hours, extracted by using AcOEt, washed by using water, dried by using MgSO₄, and then filtered. The solution obtained therefrom was concentrated, thereby obtaining 7.36 g (14.7 mmol) of Intermediate 671c (yield: 100%).

Synthesis of Intermediate 671d

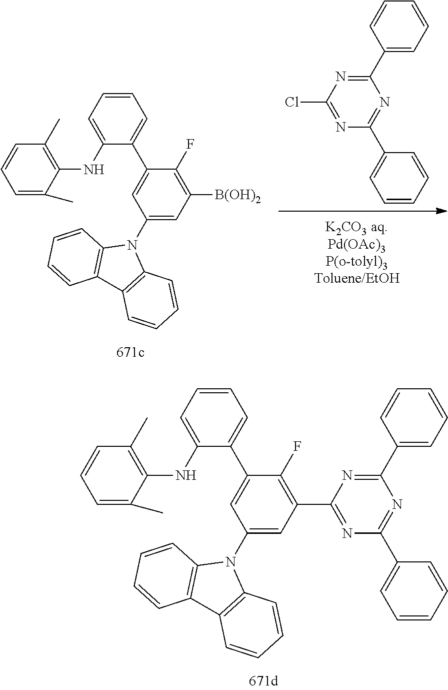 [SMILES: Cc1cccc(C)c1Nc1ccccc1-c1cc(-n2c3ccccc3c3ccccc32)cc(B(O)O)c1F ; Clc1nc(-c2ccccc2)nc(-c2ccccc2)n1 ; Cc1cccc(C)c1Nc1ccccc1-c1cc(-n2c3ccccc3c3ccccc32)cc(-c2nc(-c3ccccc3)nc(-c3ccccc3)n2)c1F]

7.36 g (14.7 mmol) of Intermediate 671c, 4.36 g (16.2 mmol) of 2-chloro-4,6-diphenyl-1,3,5-triazine, 4.06 g (29.4 mmol) of K₂CO₃, 29 ml of water, 29 ml of toluene, 29 ml of ethanol, 132 mg (0.588 mmol) of Pd(OAc)₂, and 179 mg (0.588 mmol) of P(o-tolyl)₃ were added to a three-neck flask, and the mixture was stirred at a temperature of 90° C. for 4 hours in a nitrogen atmosphere. After the reaction was completed, the mixture was cooled to room temperature, extracted by using AcOEt, washed by using water, and dried by using MgSO₄. A resultant obtained therefrom was filtered and concentrated by using a silica gel pad, and purified by silica gel chromatography (hexane:CH₂Cl₂=6:4), thereby obtaining 5.58 g (8.5 mmol) of Intermediate 671d (yield: 58%).

Synthesis of Compound 671

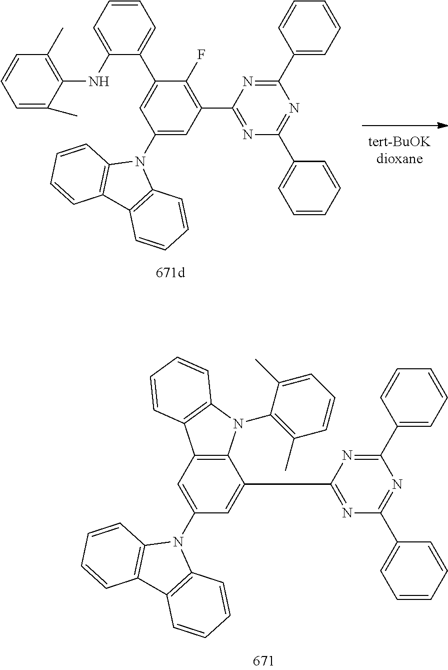 [SMILES: Cc1cccc(C)c1Nc1ccccc1-c1cc(-n2c3ccccc3c3ccccc32)cc(-c2nc(-c3ccccc3)nc(-c3ccccc3)n2)c1F ; Cc1cccc(C)c1-n1c2ccccc2c2cc(-n3c4ccccc4c4ccccc43)cc(-c3nc(-c4ccccc4)nc(-c4ccccc4)n3)c21]

5.85 g (8.5 mmol) of Intermediate 671d and 85 ml of dioxane were added to a three-neck flask, and 1.90 g (17 mmol) of potassium-tert-butoxide was added thereto. The mixture was stirred at a temperature of 110° C. for 4 hours in a nitrogen atmosphere, and then, was cooled to room temperature, diluted with 200 ml of toluene, filtered by using celite, and filtered and concentrated by using a silica gel pad. A resultant obtained therefrom was purified by silica gel chromatography (hexane:CH₂Cl₂=6:4), and a recrystallization thereof was performed by using a AcOEt/hexane mixed solvent, and then, vacuum-dried at a temperature of 60° C. for 12 hours, thereby obtaining 2.71 g (4.06 mmol) of Compound 671 (yield: 48%).

1H-NMR (300 MHz, CD₂Cl₂): 8.5-8.6 (5H), 8.2-8.3 (3H), 7.9 (1H), 7.2-7.6 (14H), 6.9 (1H), 6.7 (1H), 6.4-6.5 (2H), 1.9 (6H)

MALDI-MS Calcd: 667.27, Found: 668.2.

### Synthesis Example 9: Synthesis of Compound 670

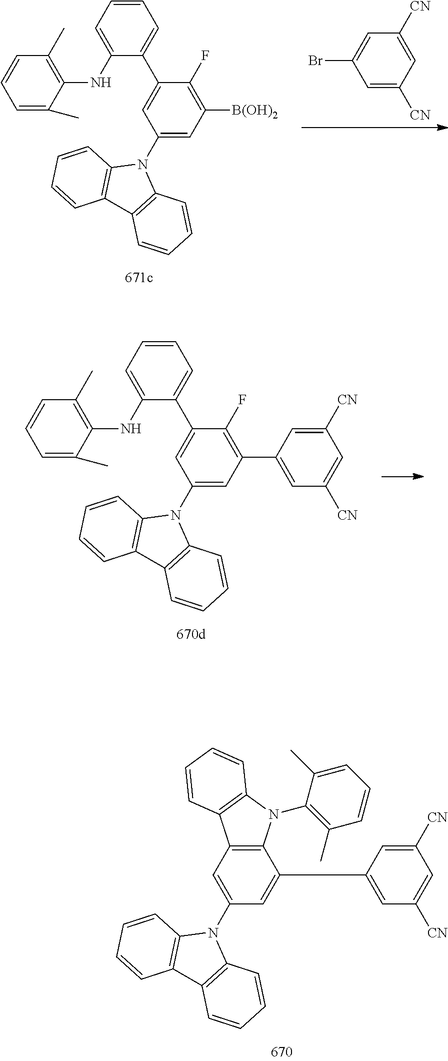 [SMILES: Cc1cccc(C)c1Nc1ccccc1-c1cc(-n2c3ccccc3c3ccccc32)cc(B(O)O)c1F ; N#Cc1cc(Br)cc(C#N)c1 ; Cc1cccc(C)c1Nc1ccccc1-c1cc(-n2c3ccccc3c3ccccc32)cc(-c2cc(C#N)cc(C#N)c2)c1F ; Cc1cccc(C)c1-n1c2ccccc2c2cc(-n3c4ccccc4c4ccccc43)cc(-c3cc(C#N)cc(C#N)c3)c21]

Synthesis of Intermediate 670d

Intermediate 670d was synthesized in the same manner as Intermediate 671d of Synthesis Example 8, except that 5-bromobenzene-1,3-dinitrile was used instead of 6-diphenyl-1,3,5-triazine.

Synthesis of Compound 670

3.5 g (yield: 56%) of Compound 670 was synthesized in the same manner as Compound 671 of Synthesis Example 8, except that Intermediate 670d was used instead of Intermediate 671d.

1H-NMR (300 MHz, CD₂Cl₂): 8.5-8.6 (2H), 8.1-8.3 (3H), 7.1-7.9 (15H), 1.9 (6H)

MALDI-MS Calcd: 562.22, Found: 567.2.

### Synthesis Example 10: Synthesis of Compound 73

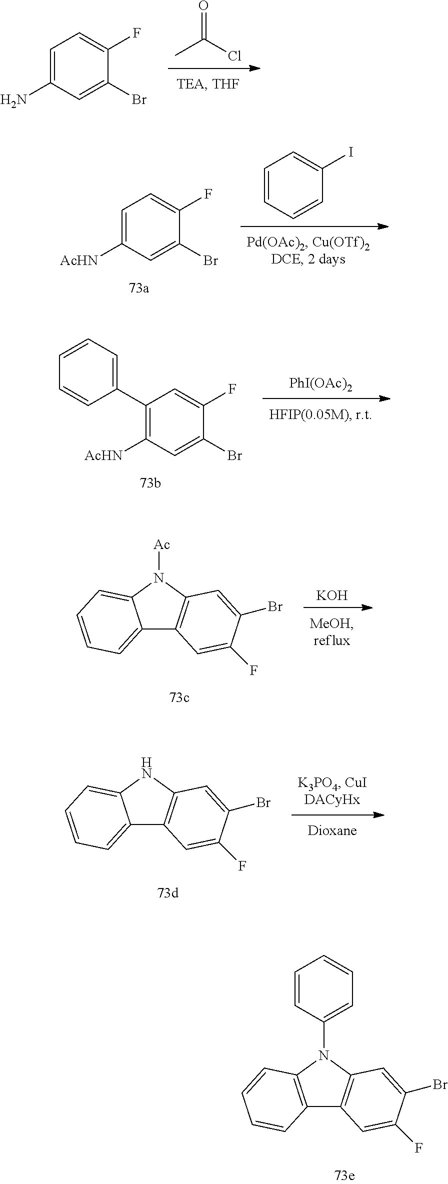

Synthesis of Intermediate 73a

3-bromo-4-fluoraniline, acetyl chloride, and THF were mixed together for a reaction. The mixture was cooled to room temperature, precipitated by using methanol, and purified, thereby obtaining Intermediate 73a.

Synthesis of Intermediate 73b

Intermediate 73a, catalysts (Pd(OAc)₂ and Cu(OTf)₂)), and 1,2-dichloroethane (DCE) were mixed together, and the mixture was stirred at a temperature of 80° C. for two days. After the reaction was completed, a resultant was diluted with dichloromethane, filtered and concentrated by using a silica filter, and then, purified by column chromatography and vacuum-dried, thereby obtaining Intermediate 73b (48%).

Synthesis of Intermediate 73c

0.15 mmol of Intermediate 73b, 1.5 mL of 0.05 M 1,1,1,3,3,3-hexafluoropropan-2-ol, 1.5 mL of dichlomethane, 7.07 mg (0.015 mmol) of PhI(OAc)₂, and peracetic acid (39% in acetic acid, 50.8 microliters (μL), 0.3 mmol) were mixed together for a reaction at room temperature for 16 hours. After the reaction was completed, the mixture was extracted by using sodium thiosulphate and dichloromethane, and an organic solvent thereof was vacuum-dried. A resultant obtained therefrom was purified by column chromatography, thereby obtaining Intermediate 73c (yield: 76%).

Synthesis of Intermediate 73d

An excessive amount of KOH and methanol were added to Intermediate 73c for a reaction at a temperature of 60° C. for 6 hours, thereby obtaining Intermediate 73d (79%).

Synthesis of Intermediate 73e

4.54 mmol of Intermediate 73d, 4.54 mmol of iodobenzene, 7.57 mmol of K₃PO₄, 1.14 mmol of CuI, 1.14 mmol of trans-1,2-diaminocyclohexane, and 20 mL of dioxane were mixed together for a reaction at a temperature of 80° C. for 12 hours. Afterwards, the mixture was cooled to room temperature, diluted with toluene, extracted by using brine and water, and then, concentrated. A resultant obtained therefrom was purified by column chromatography and vacuum-dried, thereby obtaining Intermediate 73e (yield: 84%).

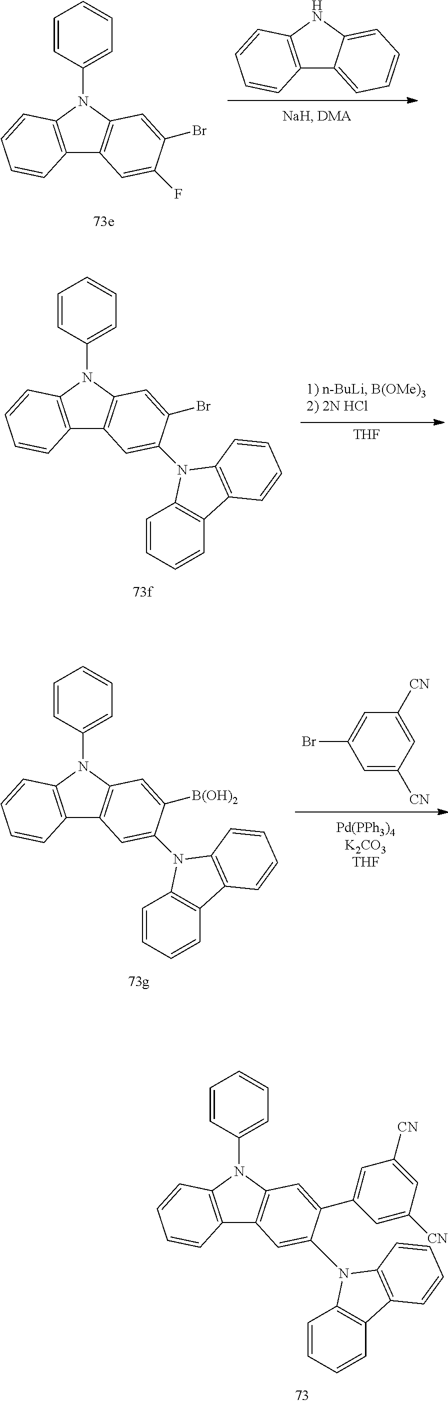

Synthesis of Intermediate 73f

5.88 mmol of NaH was vacuum-dried and mixed with 30 mL of dimethylacetamide (DMA), and the mixture was placed in a nitrogen atmosphere. Next, a mixed solution of 10 mL of DMA and 4.41 mmol of carbazole was slowly added to a flask containing NaH, and the mixture was stirred at room temperature for 50 minutes. Afterwards, in a different flask, 2.94 mmol of Intermediate 73e was mixed with 10 mL of DMA, and the mixture was added to a flask containing NaH for a reaction at room temperature for 8 hours. After the reaction was completed, ice water was added to the reactant, and accordingly, a precipitate was formed, filtered, washed by using n-hexane, and then, filtered again. A resultant obtained therefrom was purified by column chromatography and recrystallized, thereby obtaining Intermediate 73f (yield: 36%).

Synthesis of Intermediate 73g

Intermediate 73g (yield: 48%) was synthesized in the same manner as Intermediate 3a of Synthesis Example 4, except that Intermediate 73f was used instead of 9-(4-fluorophenyl)-9H-carbazole, and then, used for the next step as it is.

Synthesis of Compound 73

2.21 mmol of 5-bromoisophthalonitrile, 4.42 mmol of potassium carbonate, 11 ml of water, 11 ml of THF, and 0.11 mmol of Pd(PPh₃)₄ were added to a three-neck flask containing Intermediate 73g, and the mixture was stirred under reflux for 8 hours in a nitrogen atmosphere. After the reaction was completed, the mixture was cooled to room temperature, and methanol was added to the mixture to precipitate a solid. The precipitated solid was filtered and collected, and a filtrate was dissolved in dichloromethane and filtered and concentrated by using a silica gel pad. Then, a recrystallization thereof was performed by using AcOEt, thereby obtaining Compound 73 (yield: 70%).

MALDI-MS Calcd: 534.6, Found: 535.6.

### Synthesis Example 11: Synthesis of Compound 905

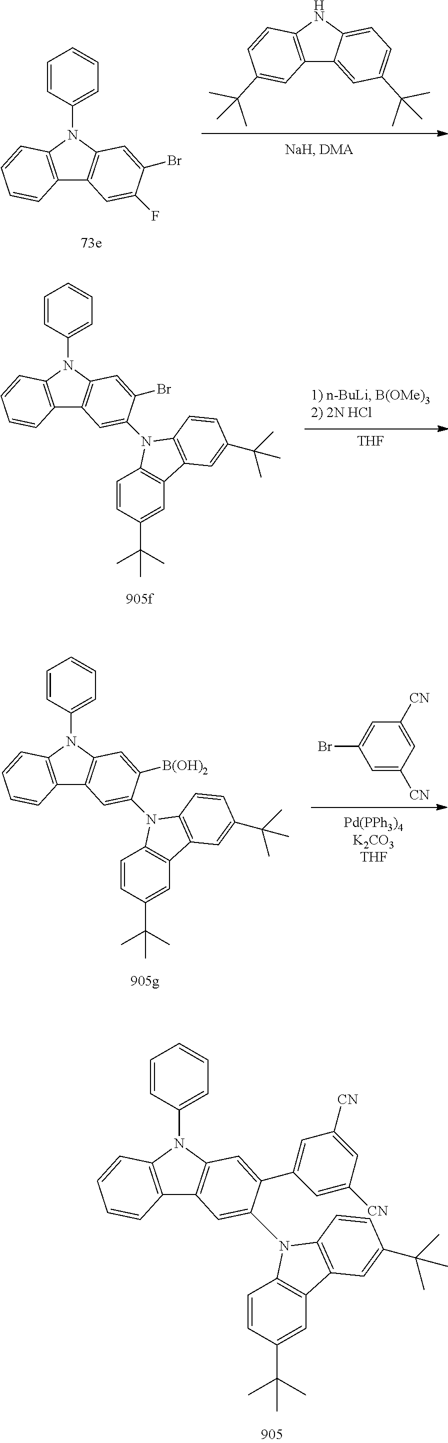

Synthesis of Intermediate 905f

Intermediate 905f was synthesized in the same manner as Intermediate 73f of Synthesis Example 10, except that 3,6-di-tert-butyl-9H-carbazole was used instead of carbazole.

Synthesis of Intermediate 905g

Intermediate 905g was synthesized in the same manner as Intermediate 73g of Synthesis Example 10, except that Intermediate 905f was used instead of Intermediate 73f.

Synthesis of Compound 905

Compound 905 (yield: 70%) was synthesized in the same manner as Compound 73 of Synthesis Example 10, except that Intermediate 905g was used instead of Intermediate 73g.

MALDI-MS Calcd: 646.31, Found: 646.8.

### Synthesis Example 12: Synthesis of Compound 863

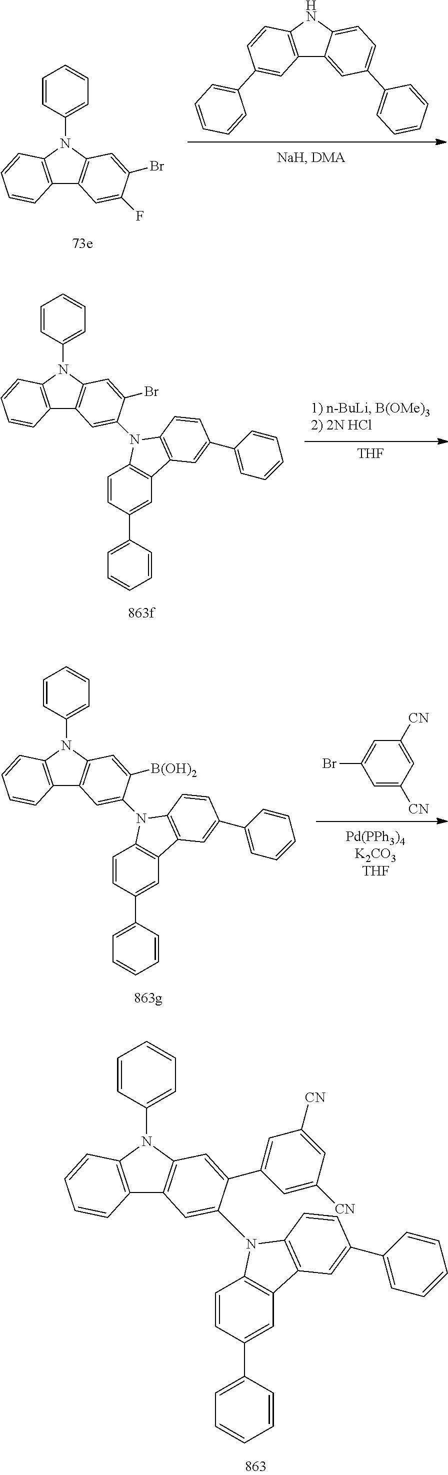

Synthesis of Intermediate 863f

Intermediate 863f was synthesized in the same manner as Intermediate 73f of Synthesis Example 10, except that 3,6-diphenyl-9H-carbazole was used instead of carbazole.

Synthesis of Intermediate 863g

Intermediate 863g was synthesized in the same manner as Intermediate 73g of Synthesis Example 10, except that Intermediate 863f was used instead of Intermediate 73f.

Synthesis of Compound 863

Compound 863 (yield: 70%) was synthesized in the same manner as Compound 73 of Synthesis Example 10, except that Intermediate 863g was used instead of Intermediate 73g.

MALDI-MS Calcd: 686.25, Found: 686.8.

### Synthesis Example 13: Synthesis of Compound 5

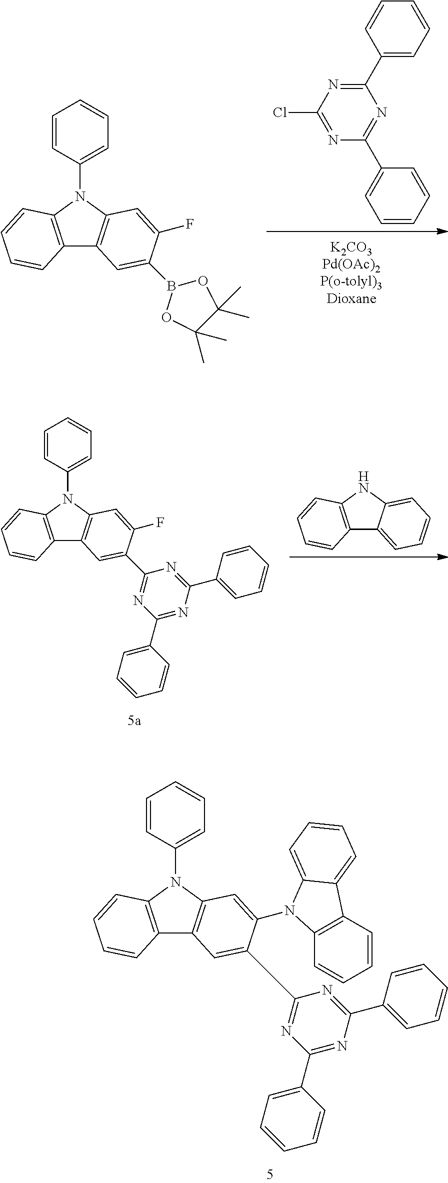

Synthesis of Intermediate 5a

Intermediate 5a was synthesized in the same manner as Intermediate 3d of Synthesis Example 4, except that 2-fluoro-3-(4,4,5,5-tetramethyl-1,3,2-dioxaborolan-2-yl)-9-phenyl-9H-carbazole was used instead of Intermediate 3c.

Synthesis of Compound 5

0.98 g (yield: 70%) of Compound 5 was synthesized in the same manner as Compound 1 of Synthesis Example 1, except that Intermediate 5a was used instead of Intermediate 1a.

1H-NMR (300 MHz, CD₂Cl₂): 8.6 (1H), 8.45 (1H), 8.2 (1H), 8.08 (2H), 8.0-7.9 (4H), 7.85-7.65 (4H), 7.65-7.52 (3H), 7.52-7.15 (13H)

MALDI-MS Calcd: 639.24, Found: 639.26.

### Synthesis Example 14: Synthesis of Compound 20

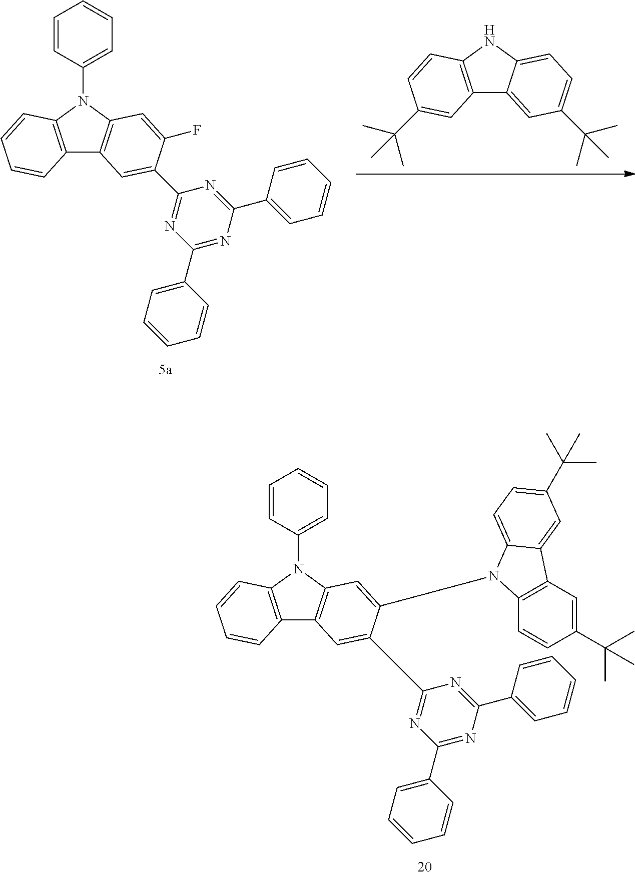 [SMILES: Fc1cc2c(cc1-c1nc(-c3ccccc3)nc(-c3ccccc3)n1)c1ccccc1n2-c1ccccc1 ; CC(C)(C)c1ccc2[nH]c3ccc(C(C)(C)C)cc3c2c1 ; CC(C)(C)c1ccc2c(c1)c1cc(C(C)(C)C)ccc1n2-c1cc2c(cc1-c1nc(-c3ccccc3)nc(-c3ccccc3)n1)c1ccccc1n2-c1ccccc1]

0.5 g (yield: 33%) of Compound 20 was synthesized in the same manner as Compound 5 of Synthesis Example 13, except that 3,6-di-tert-butyl-9H-carbazole was used instead of carbazole.

1H-NMR (300 MHz, CD₂Cl₂): 8.6 (1H), 8.45 (1H), 8.2 (1H), 8.08 (2H), 8.0-7.9 (4H), 7.85-7.65 (4H), 7.65-7.52 (3H), 7.5-6.8 (11H), 1.43 (18H)

MALDI-MS Calcd: 751.37, Found: 752.3.

### Synthesis Example 15: Synthesis of Compound 36

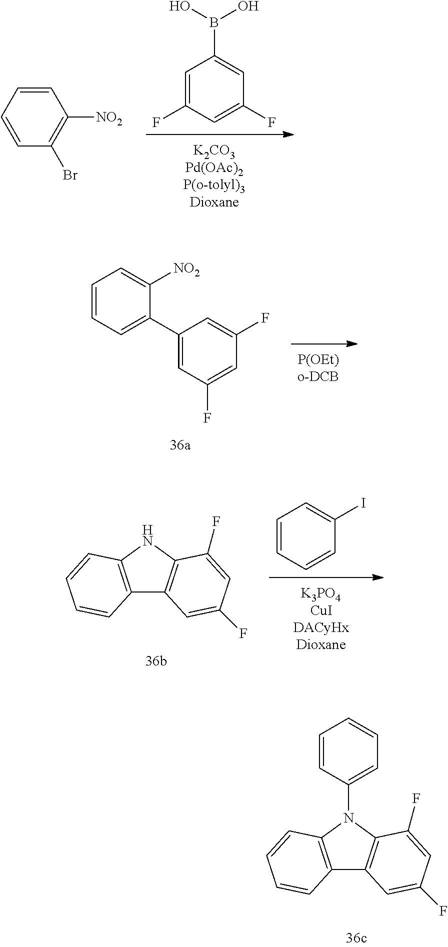 [SMILES: O=[N+]([O-])c1ccccc1Br ; OB(O)c1cc(F)cc(F)c1 ; O=[N+]([O-])c1ccccc1-c1cc(F)cc(F)c1 ; Fc1cc(F)c2[nH]c3ccccc3c2c1 ; Ic1ccccc1 ; Fc1cc(F)c2c(c1)c1ccccc1n2-c1ccccc1]

Synthesis of Intermediate 36a

Intermediate 36a was synthesized in the same manner as in the Suzuki coupling reaction of Synthesis Example 1 by using 1-bromo-2nitrobenzene and (3,5-difluorophenyl)boronic acid as starting materials.

Synthesis of Intermediate 36b

0.1 mmol of Intermediate 36a, 0.05 M of P(OEt), and o-dichlorobenzene were mixed, and the mixture was stirred under reflux for 12 hours. After the reaction was completed, a resultant was extracted by suing CH₂Cl₂ and brine, and an organic solvent thereof was removed under reduced pressure, thereby obtaining Intermediate 36b. Then, Intermediate 36b was used for next steps.

Synthesis of Intermediate 36c

Intermediate 36c was synthesized in the same manner as Intermediate 73e of Synthesis Example 10, except that Intermediate 36b was used instead of Intermediate 73d.

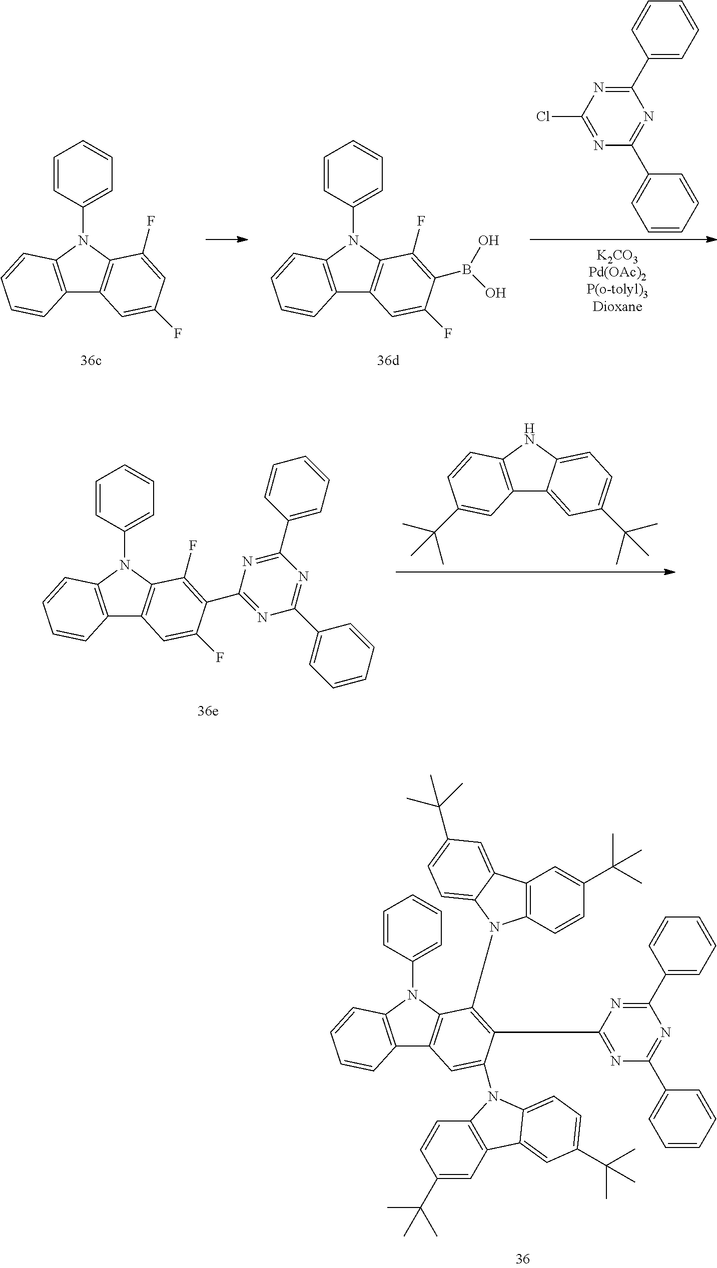 [SMILES: Fc1cc(F)c2c(c1)c1ccccc1n2-c1ccccc1 ; OB(O)c1c(F)cc2c3ccccc3n(-c3ccccc3)c2c1F ; Clc1nc(-c2ccccc2)nc(-c2ccccc2)n1 ; Fc1cc2c3ccccc3n(-c3ccccc3)c2c(F)c1-c1nc(-c2ccccc2)nc(-c2ccccc2)n1 ; CC(C)(C)c1ccc2[nH]c3ccc(C(C)(C)C)cc3c2c1 ; CC(C)(C)c1ccc2c(c1)c1cc(C(C)(C)C)ccc1n2-c1cc2c3ccccc3n(-c3ccccc3)c2c(-n2c3ccc(C(C)(C)C)cc3c3cc(C(C)(C)C)ccc32)c1-c1nc(-c2ccccc2)nc(-c2ccccc2)n1]

Synthesis of Intermediate 36d and Intermediate 36e

Intermediate 36d and Intermediate 36e were sequentially synthesized in the same manner as in Synthesis Example 1, except that Intermediate 36c was used instead of 3-fluoro-N-phenylcarbazole.

Synthesis of Compound 36

0.45 g (yield: 21%) of Compound 36 was synthesized in the same manner as in Synthesis Example 2, except that Intermediate 36e was used instead of Intermediate 1a.

MALDI-MS Calcd: 1028.55, Found: 1029.3.

### Synthesis Example 16: Synthesis of Compound 936

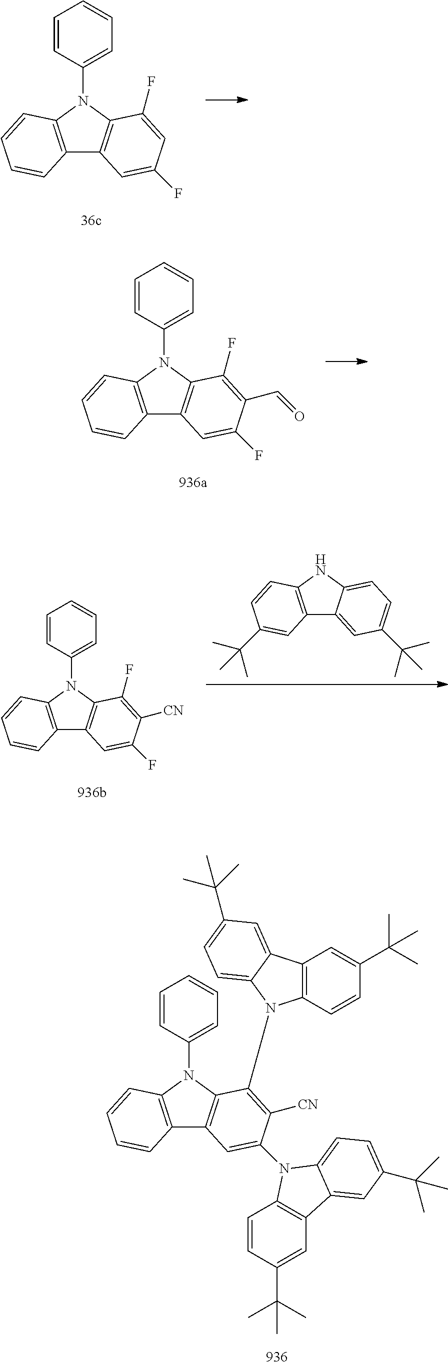 [SMILES: Fc1cc(F)c2c(c1)c1ccccc1n2-c1ccccc1 ; O=Cc1c(F)cc2c3ccccc3n(-c3ccccc3)c2c1F ; N#Cc1c(F)cc2c3ccccc3n(-c3ccccc3)c2c1F ; CC(C)(C)c1ccc2[nH]c3ccc(C(C)(C)C)cc3c2c1 ; CC(C)(C)c1ccc2c(c1)c1cc(C(C)(C)C)ccc1n2-c1cc2c3ccccc3n(-c3ccccc3)c2c(-n2c3ccc(C(C)(C)C)cc3c3cc(C(C)(C)C)ccc32)c1C#N]

Synthesis of Intermediate 936a

10.05 g (36 mmol) of Intermediate 36c, 6.61 g (46.8 mmol) of trimethyl phosphate, and 180 mL of THF were mixed cooled to a temperature of −60° C. in a nitrogen atmosphere. Afterwards, 15.8 mL (39.6 mmol) of n-BuLi was added thereto, and the mixture was stirred for 5 minutes. 10 mL of DMF was additionally added dropwise thereto, stirred at a temperature of −60° C. for 30 minutes, and the mixture was stirred again for 3 hours after the temperature was raised to room temperature. After the reaction was completed, 10 mL of water was added thereto, and the mixture was stirred. A resultant extracted by using CH₂Cl₂ was dried by using MgSO₄, filtered and concentrated, and vacuum-dried, thereby obtaining 10.5 g (91%) of Intermediate 936a.

Synthesis of Intermediate 936b

36 mmol of Intermediate 936a, 108 mmol of Ac₂O, 108 mmol of potassium carbonate, and 144 mL of DMSO were mixed, and the mixture was stirred at a temperature of 90° C. for 3 hours. After the reaction was completed, a resultant extracted by dilution using toluene was concentrated, purified by column chromatography, and recrystallized, thereby obtaining Intermediate 936b (63%).

Synthesis of Compound 936

0.58 g (yield: 23%) of Compound 936 was synthesized in the same manner as in Synthesis Example 2, except that Intermediate 936b was used instead of Intermediate 1a.

MALDI-MS Calcd: 822.47, Found: 823.4.

### Evaluation Example 1: Evaluation of Stoke's Shift Energy Level and Photoluminescence Spectrum

Photoluminescence (PL) spectra of the compounds shown in Table 2 and a UV absorption spectrum of Compound FD(5) were measured by using methods described in Table 1 below.

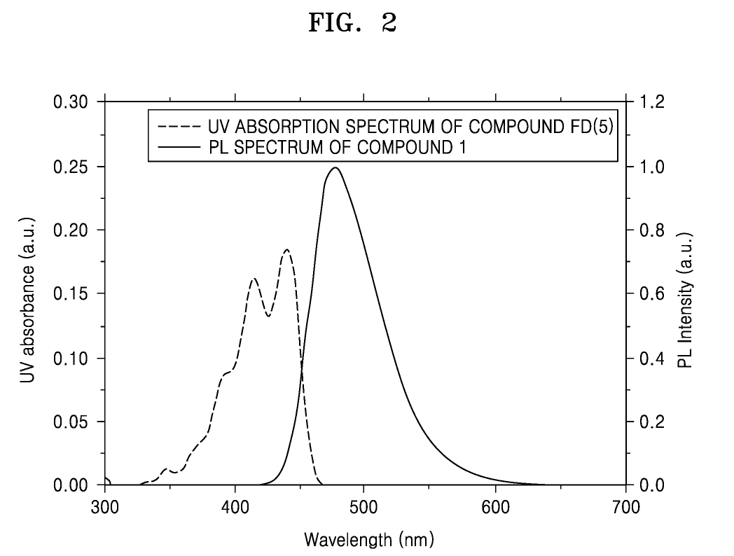

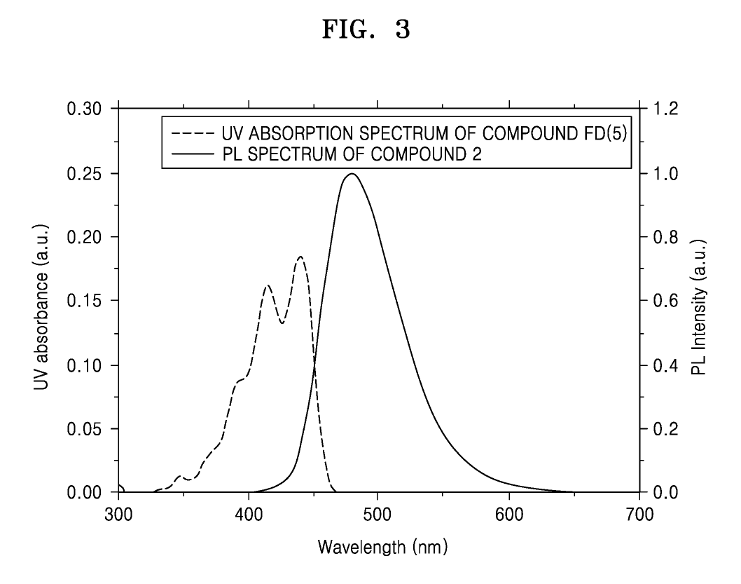

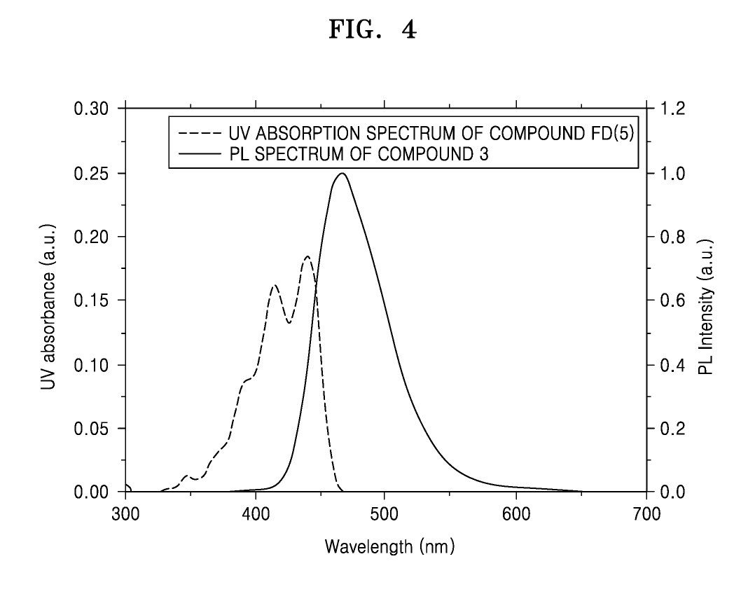

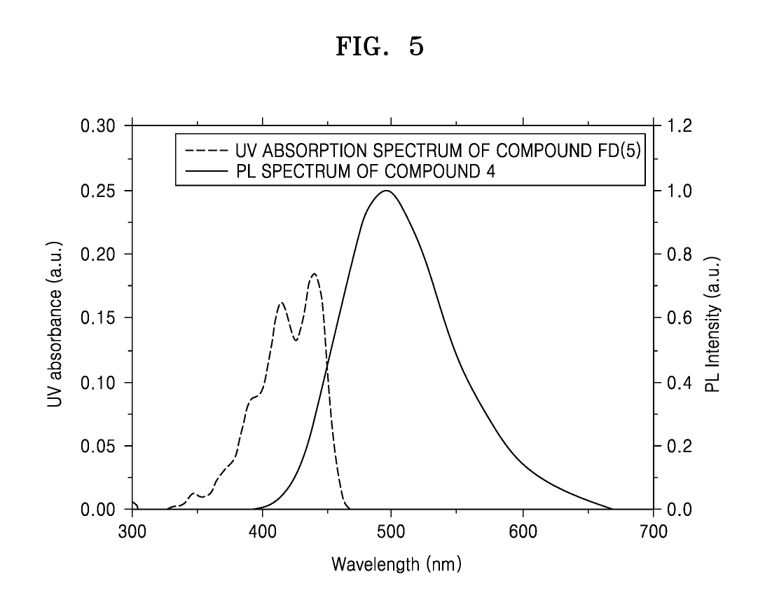

Then, an onset wavelength in the PL spectrum of Compound 1 and a maximum emission wavelength in the PL spectrum of Compound 1 were converted in units of electron volt (eV), a difference therebetween was calculated, and a Stoke's shift (eV) value of Compound 1 was evaluated. This was repeated on the rest of the compounds, and results thereof are shown in Table 2 below. Also, the UV absorption spectrum of Compound FD(5) and the PL spectrum of Compound 1 are shown in FIG. 2, the UV absorption spectrum of Compound FD(5) and the PL spectrum of Compound 2 are shown in FIG. 3, the UV absorption spectrum of Compound FD(5) and the PL spectrum of Compound 3 are shown in FIG. 4, and the UV absorption spectrum of Compound FD(5) and the PL spectrum of Compound 4 are shown in FIG. 5.

TABLE 1

| | |
|---|---|
| UV | Each Compound is diluted with toluene to a concentration |
| absorption | of 10⁻⁵ M, and a UV absorption spectrum is measured |
| spectrum | by using Shimadzu UV-350 Spectrometer. |
| PL | Each Compound is diluted with toluene to a concentration |
| spectrum | of 10⁻⁵ M, and a PL spectrum is measured by using an |
| | F700 Spectrofluorometer with a xenon lamp, which is |
| | manufactured by Hitachi, (@ 298 K). |

TABLE 2

| | Emission onset | Maximum emission | Stoke's |
|---|---|---|---|
| Compound No. | wavelength (nm) | wavelength (nm) | shift (eV) |
| Compound 1 | 432 | 477 | 0.27 |
| Compound 2 | 432 | 479 | 0.28 |
| Compound 3 | 421 | 466 | 0.28 |
| Compound 4 | 411 | 451 | 0.26 |
| Compound 50 | 437 | 484 | 0.27 |
| Compound 669 | 432 | 479 | 0.28 |
| Compound 671 | 411 | 454 | 0.28 |
| Compound 670 | 400 | 440 | 0.28 |
| Compound 73 | 398 | 435 | 0.26 |
| Compound 905 | 410 | 448 | 0.25 |
| Compound 863 | 411 | 450 | 0.26 |
| Compound 5 | 402 | 444 | 0.29 |
| Compound 20 | 416 | 460 | 0.28 |
| Compound 36 | 434 | 483 | 0.29 |
| Compound 936 | 410 | 440 | 0.21 |
| Compound A | 401 | 495 | 0.59 |

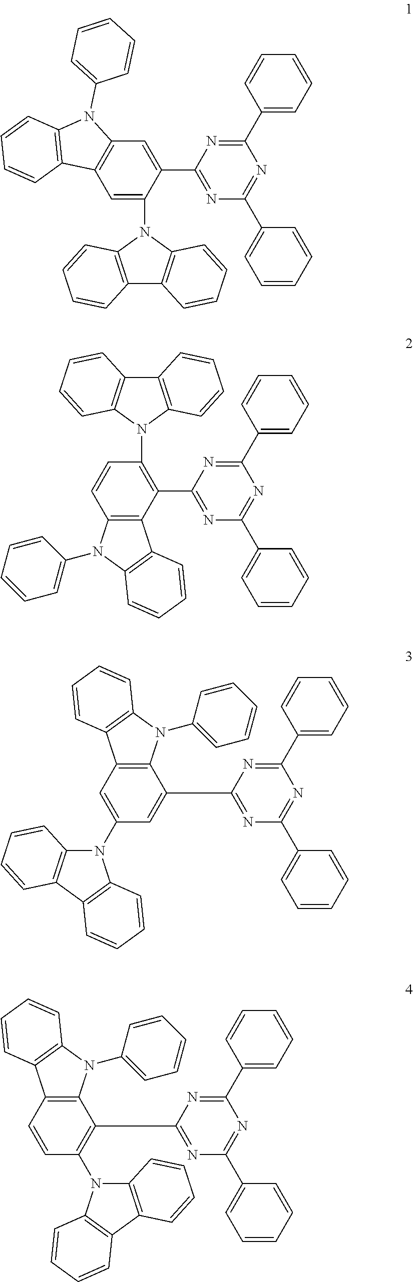

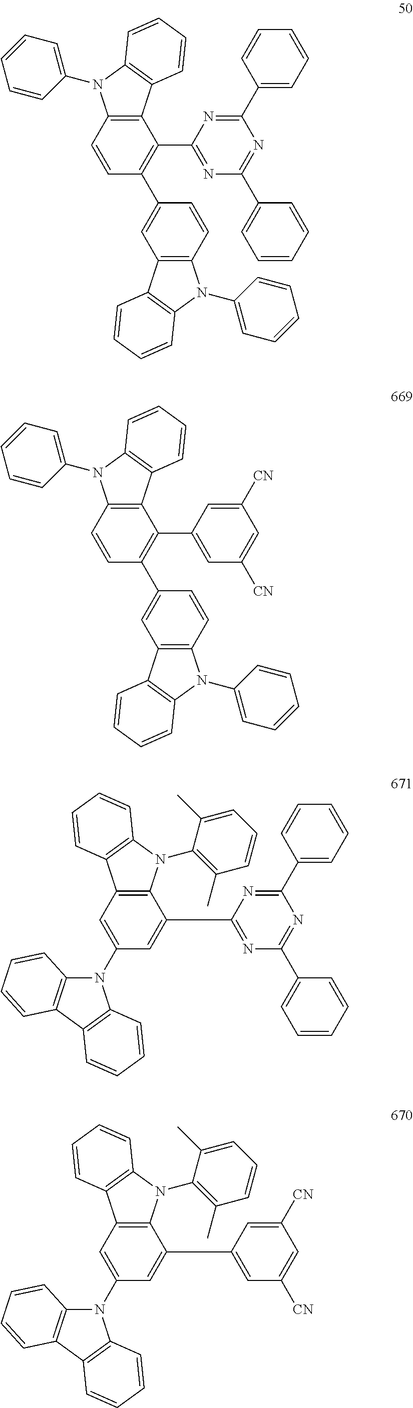 [SMILES: c1ccc(-c2nc(-c3ccccc3)nc(-c3c(-c4ccc5c(c4)c4ccccc4n5-c4ccccc4)ccc4c3c3ccccc3n4-c3ccccc3)n2)cc1 ; N#Cc1cc(C#N)cc(-c2c(-c3ccc4c(c3)c3ccccc3n4-c3ccccc3)ccc3c2c2ccccc2n3-c2ccccc2)c1 ; Cc1cccc(C)c1-n1c2ccccc2c2cc(-n3c4ccccc4c4ccccc43)cc(-c3nc(-c4ccccc4)nc(-c4ccccc4)n3)c21 ; Cc1cccc(C)c1-n1c2ccccc2c2cc(-n3c4ccccc4c4ccccc43)cc(-c3cc(C#N)cc(C#N)c3)c21]

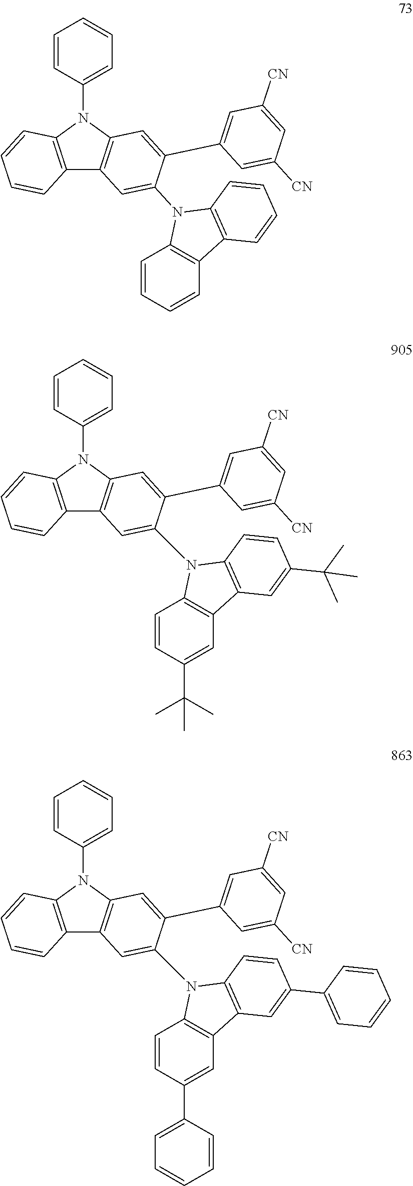 [SMILES: N#Cc1cc(C#N)cc(-c2cc3c(cc2-n2c4ccccc4c4ccccc42)c2ccccc2n3-c2ccccc2)c1 ; CC(C)(C)c1ccc2c(c1)c1cc(C(C)(C)C)ccc1n2-c1cc2c3ccccc3n(-c3ccccc3)c2cc1-c1cc(C#N)cc(C#N)c1 ; N#Cc1cc(C#N)cc(-c2cc3c(cc2-n2c4ccc(-c5ccccc5)cc4c4cc(-c5ccccc5)ccc42)c2ccccc2n3-c2ccccc2)c1]

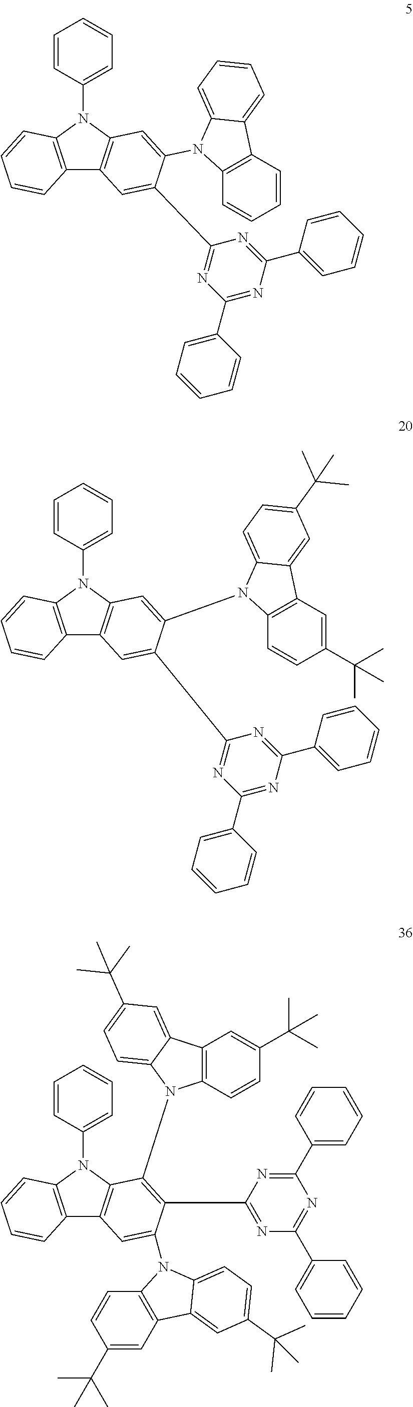 [SMILES: c1ccc(-c2nc(-c3ccccc3)nc(-c3cc4c5ccccc5n(-c5ccccc5)c4cc3-n3c4ccccc4c4ccccc43)n2)cc1 ; CC(C)(C)c1ccc2c(c1)c1cc(C(C)(C)C)ccc1n2-c1cc2c(cc1-c1nc(-c3ccccc3)nc(-c3ccccc3)n1)c1ccccc1n2-c1ccccc1 ; CC(C)(C)c1ccc2c(c1)c1cc(C(C)(C)C)ccc1n2-c1cc2c3ccccc3n(-c3ccccc3)c2c(-n2c3ccc(C(C)(C)C)cc3c3cc(C(C)(C)C)ccc32)c1-c1nc(-c2ccccc2)nc(-c2ccccc2)n1]

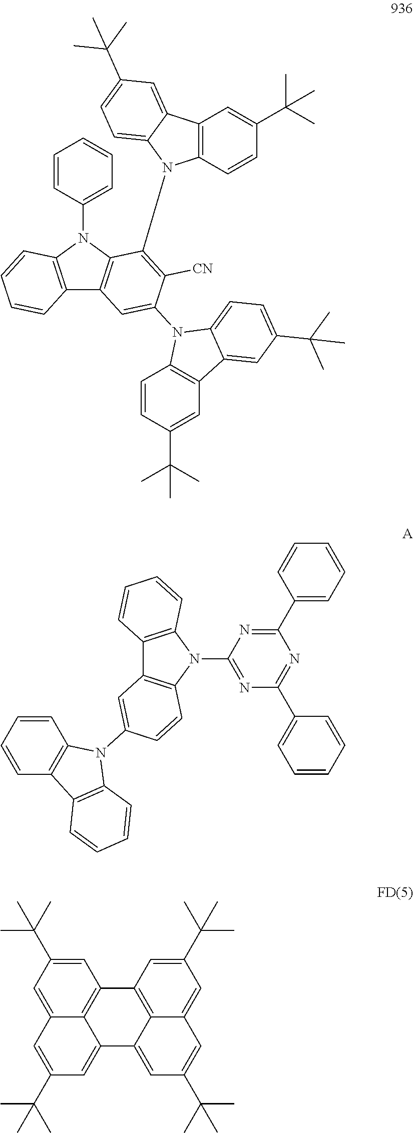

Referring to Table 2, it was confirmed that the Stoke's shift energy levels of Compounds 1 to 4, 50, 669, 671, 670, 73, 905, 863, 5, 20, 36, and 936 were smaller than the Stoke's shift energy level of Compound A.

Also, referring to FIGS. 2 to 5, it can be confirmed that an overlap region between the PL spectrum of each of Compounds 1 to 4 and the UV absorption spectrum of Compound FD(5) is relatively wide, and an energy transfer between each of Compounds 1 to 4 and Compound FD(5) is effectively achieved.

### Example 1

A glass substrate, on which an ITO electrode (first electrode, anode) having a thickness of 1,500 Å was formed, was sonicated with distilled water. After the sonicating with distilled water was completed, the glass substrate was ultrasonically cleaned by sequentially using iso-propyl alcohol, acetone, and methanol, was dried, and then transferred to a plasma cleaner. Then, the glass substrate was cleaned for 5 minutes by using oxygen plasma and was provided to a vacuum deposition apparatus.

Compound HT3 and Compound HP-1 were co-deposited on the ITO electrode of the glass substrate to form a hole injection layer having a thickness of 100 Å, Compound HT3 was deposited on the hole injection layer to form a hole transport layer having a thickness of 1,300 Å, mCP was deposited on the hole transport layer to form an electron blocking layer having a thickness of 150 Å, thereby forming a hole transport region.

Compound H19 (host) and Compound 1 (dopant) were co-deposited on the hole transport region at a volume ratio of 85:15 to form an emission layer having a thickness of 300 Å.

Compound BCP was vacuum-deposited on the emission layer to form a hole blocking layer having a thickness of 100 Å, Compound ET3 and LiQ were vacuum-deposited on the hole blocking layer to form an electron transport layer having a thickness of 250 Å, LiQ was deposited on the electron transport layer to form an electron injection layer having a thickness of 5 Å, and Al was deposited on the electron injection layer to form a second electrode (cathode) having a thickness of 1,000 Å, thereby completing the manufacture of an organic light-emitting device.

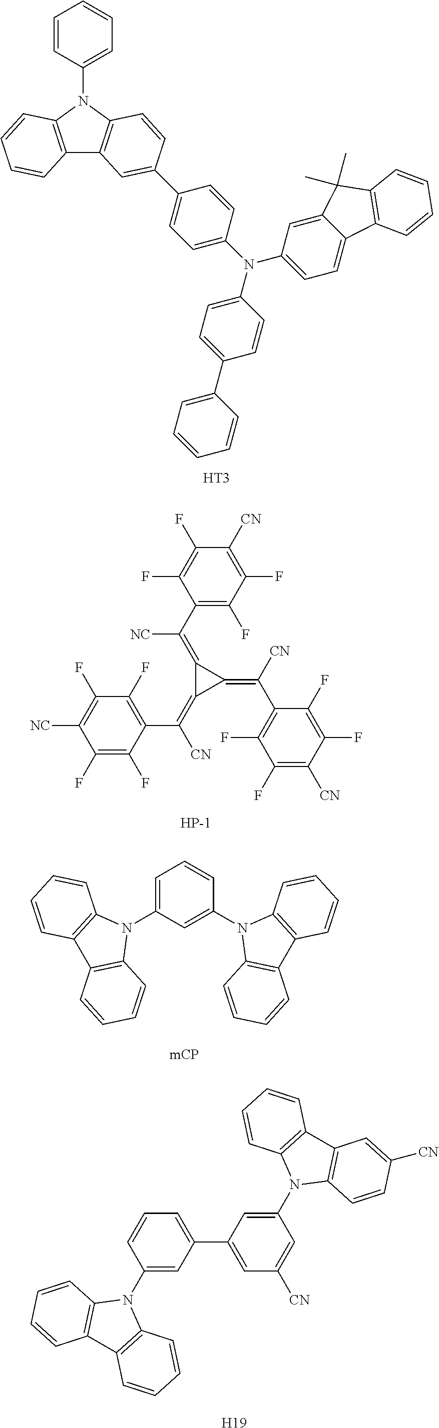

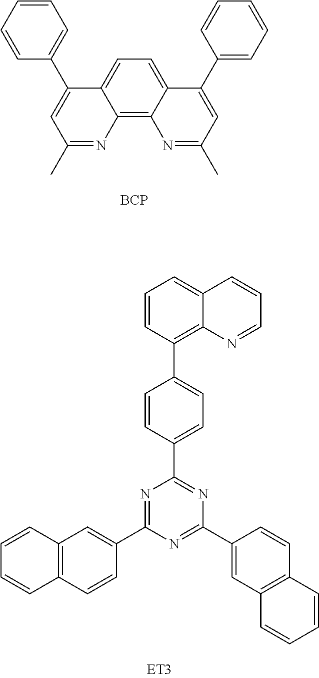

### Examples 2 to 10 and Comparative Examples 1 to 3

Organic light-emitting devices of Examples 2 to 10 and Comparative Examples 1 to 3 were manufactured in the same manner as in Example 1, except that Compounds shown in Table 3 were each used instead of Compound 1 as a dopant in forming an emission layer.

### Evaluation Example 1

The driving voltage, maximum emission wavelength, and color purity of the organic light-emitting devices manufactured according to Examples 1 to 10 and Comparative Examples 1 to 3 were measured by using a current-voltage meter (Keithley 2400) and a luminance meter (Minolta Cs-1000A) (at 500 cd/m²), and results thereof are shown in Tables 3 and 4 below.

The lifespan (T₉₅) of the organic light-emitting devices manufactured according to Examples 1 to 10 and Comparative Examples 1 to 3 was evaluated, and results thereof are shown in Table 4 below. The lifespan (T₉₅) data (at 500 candelas per square meter, cd/m²) was obtained by evaluating an amount of time (hr) that lapsed when luminance was 95% of initial luminance (100%). The lifespan (T₉₅) was provided in a relative manner with respect to those of the organic light-emitting device of Comparative Examples 1 and 2 (1%).

TABLE 3

| | | | | Maximum |
|---|---|---|---|---|
| | | | Driving | emission |
| | | | voltage | wavelength |
| Example No. | Host | Dopant | (V) | (nm) |
| Example 1 | Compound H19 | Compound 1 | 5.0 | 473 |
| Example 2 | Compound H19 | Compound 2 | 4.8 | 471 |
| Example 3 | Compound H19 | Compound 3 | 4.9 | 464 |
| Example 4 | Compound H19 | Compound 671 | 5.2 | 447 |
| Example 5 | Compound H19 | Compound 73 | 8.2 | 431 |
| Example 6 | Compound H19 | Compound 905 | 8.53 | 438 |
| Example 7 | Compound H19 | Compound 5 | 6.5 | 443 |
| Example 8 | Compound H19 | Compound 20 | 4.6 | 467 |
| Example 9 | Compound H19 | Compound 36 | 4.4 | 481 |
| Example 10 | Compound H19 | Compound 936 | 5.86 | 445 |
| Comparative | Compound H19 | Compound A | 5.0 | 501 |
| Example 1 | | | | |
| Comparative | Compound H19 | Compound B | 9.5 | 420 |
| Example 2 | | | | |
| Comparative | Compound H19 | Compound C | 7.28 | 465 |
| Example 3 | | | | |

TABLE 4

| | | | | | LT₉₅@500 |
|---|---|---|---|---|---|
| | | | | Emis- | cd/m² (%) |
| | | | CIE | sion | (relative |
| Example No. | Host | Dopant | x, y | color | value) |
| Example 1 | Compound | Compound | 0.16, | Blue | 800 |
| | H19 | 1 | 0.26 | | |
| Example 2 | Compound | Compound | 0.16, | Blue | 200 |
| | H19 | 2 | 0.28 | | |
| Example 3 | Compound | Compound | 0.15, | Blue | 300 |
| | H19 | 3 | 0.20 | | |
| Example 4 | Compound | Compound | 0.15, | Blue | 50 |
| | H19 | 671 | 0.11 | | |
| Example 5 | Compound | Compound | 0.16, | Blue | 40 |
| | H19 | 73 | 0.07 | | |
| Example 6 | Compound | Compound | 0.16, | Blue | 40 |
| | H19 | 905 | 0.13 | | |
| Example 7 | Compound | Compound | 0.15, | Blue | 100 |
| | H19 | 5 | 0.10 | | |
| Example 8 | Compound | Compound | 0.16, | Blue | 200 |
| | H19 | 20 | 0.24 | | |
| Example 9 | Compound | Compound | 0.19, | Blue | 400 |
| | H19 | 36 | 0.37 | | |
| Example 10 | Compound | Compound | 0.15, | Blue | 20 |
| | H19 | 936 | 0.08 | | |
| Comparative | Compound | Compound | 0.25, | Bluish | 1 |
| Example 1 | H19 | A | 0.52 | Green | |
| Comparative | Compound | Compound | 0.15, | Blue | 1 |
| Example 2 | H19 | B | 0.10 | | |
| Comparative | Compound | Compound | 0.16, | Blue | 5 |
| Example 3 | H19 | C | 0.25 | | |

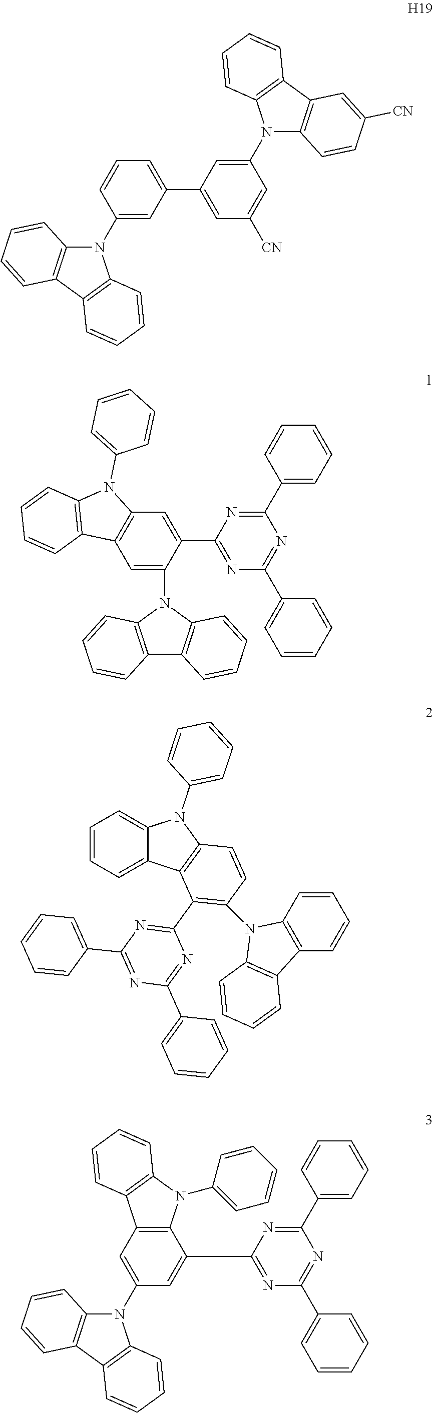

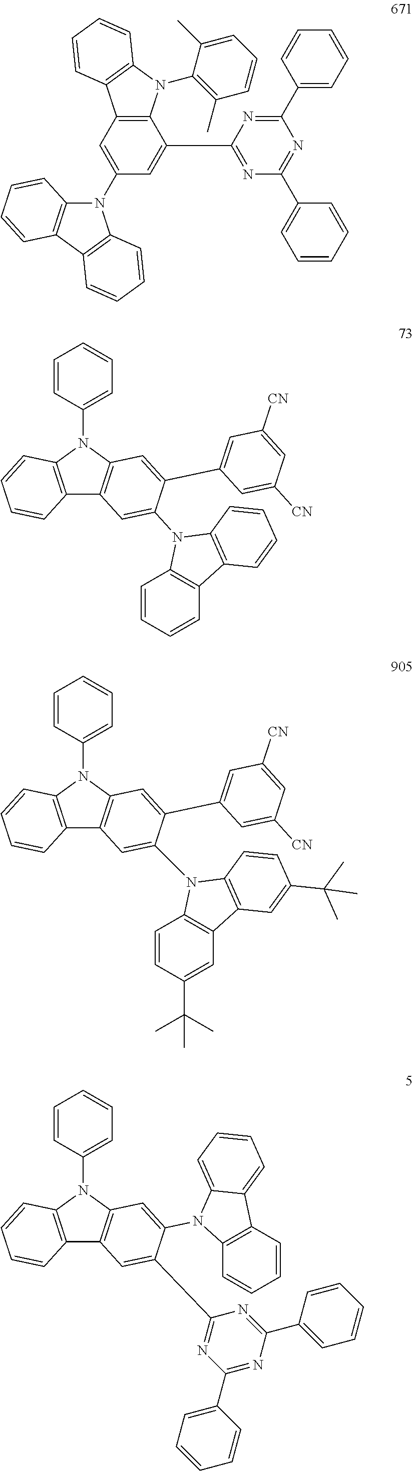 [SMILES: Cc1cccc(C)c1-n1c2ccccc2c2cc(-n3c4ccccc4c4ccccc43)cc(-c3nc(-c4ccccc4)nc(-c4ccccc4)n3)c21 ; N#Cc1cc(C#N)cc(-c2cc3c(cc2-n2c4ccccc4c4ccccc42)c2ccccc2n3-c2ccccc2)c1 ; CC(C)(C)c1ccc2c(c1)c1cc(C(C)(C)C)ccc1n2-c1cc2c3ccccc3n(-c3ccccc3)c2cc1-c1cc(C#N)cc(C#N)c1 ; c1ccc(-c2nc(-c3ccccc3)nc(-c3cc4c5ccccc5n(-c5ccccc5)c4cc3-n3c4ccccc4c4ccccc43)n2)cc1]

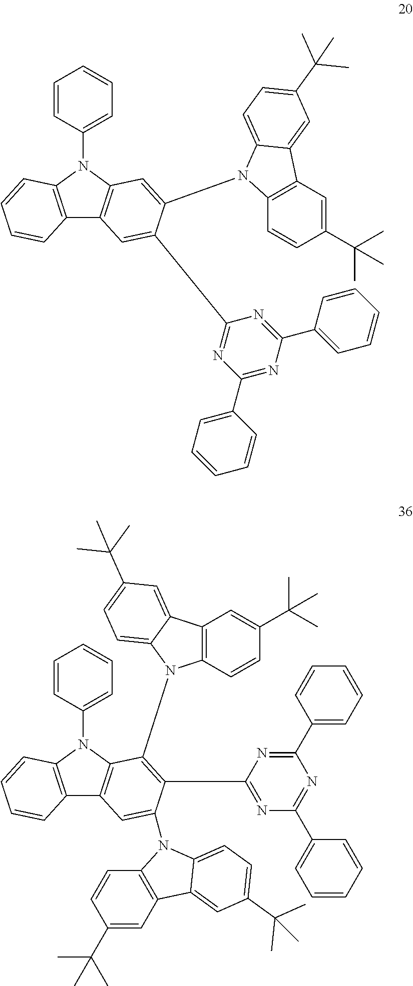 [SMILES: CC(C)(C)c1ccc2c(c1)c1cc(C(C)(C)C)ccc1n2-c1cc2c(cc1-c1nc(-c3ccccc3)nc(-c3ccccc3)n1)c1ccccc1n2-c1ccccc1 ; CC(C)(C)c1ccc2c(c1)c1cc(C(C)(C)C)ccc1n2-c1cc2c3ccccc3n(-c3ccccc3)c2c(-n2c3ccc(C(C)(C)C)cc3c3cc(C(C)(C)C)ccc32)c1-c1nc(-c2ccccc2)nc(-c2ccccc2)n1]

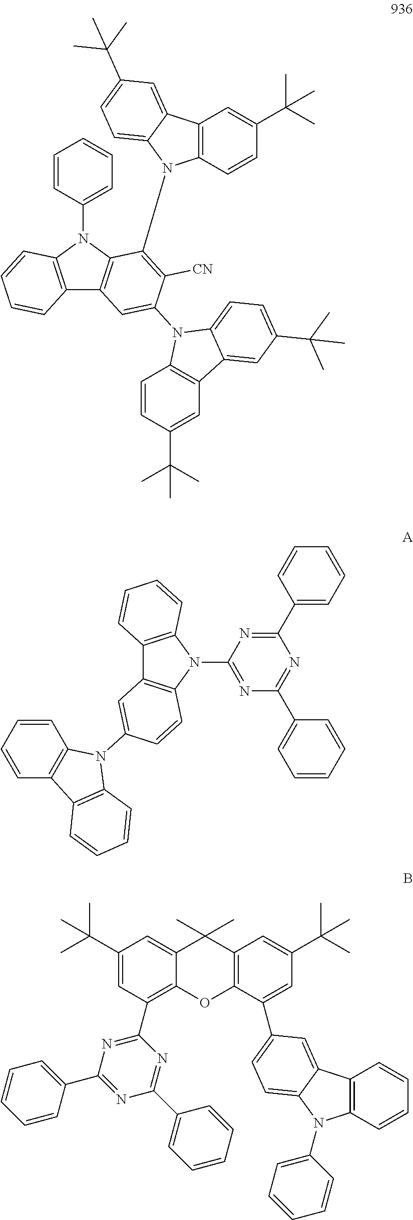 [SMILES: CC(C)(C)c1ccc2c(c1)c1cc(C(C)(C)C)ccc1n2-c1cc2c3ccccc3n(-c3ccccc3)c2c(-n2c3ccc(C(C)(C)C)cc3c3cc(C(C)(C)C)ccc32)c1C#N ; c1ccc(-c2nc(-c3ccccc3)nc(-n3c4ccccc4c4cc(-n5c6ccccc6c6ccccc65)ccc43)n2)cc1 ; CC(C)(C)c1cc(-c2ccc3c(c2)c2ccccc2n3-c2ccccc2)c2c(c1)C(C)(C)c1cc(C(C)(C)C)cc(-c3nc(-c4ccccc4)nc(-c4ccccc4)n3)c1O2]

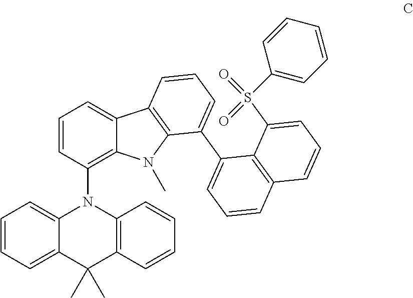 [SMILES: Cn1c2c(-c3cccc4cccc(S(=O)(=O)c5ccccc5)c34)cccc2c2cccc(N3c4ccccc4C(C)(C)c4ccccc43)c21]

From Tables 3 and 4, it can be confirmed that the driving voltage and lifespan characteristics of the organic light-emitting devices of Examples 1 to 10 are equivalent to or more improved than those of the organic light-emitting devices of Comparative Examples 2 and 3.

An organic light-emitting device according to one or more embodiments may have high efficiency and a long lifespan.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the present disclosure as defined by the following claims.

What is claimed is:

1. An organic light-emitting device comprising:
a first electrode;
a second electrode facing the first electrode; and
an organic layer disposed between the first electrode and the second electrode, wherein the organic layer comprises an emission layer,
wherein
the emission layer comprises a fluorescent compound, of which a difference between a singlet excitation energy level and a triplet excitation energy level is greater than 0 electron volts and equal to or less than 0.5 electron volts,
a proportion of fluorescent emission components with respect to total emission components emitted from the emission layer is about 90% or more, and the emission layer does not include a phosphorescent emission compound,
the fluorescent compound comprises electron donor groups in the number of n1 and electron acceptor groups in the number of n2, wherein n1 and n2 are each independently an integer from 1 to 10,
the electron donor groups in the number of n1 and the electron acceptor groups in the number of n2 are chemically bonded to each other in random order, provided that a chemical bond between the electron donor group and the electron acceptor group is a carbon-carbon single bond,
at least one of the electron donor groups in the number of n1 is an electron donor group represented by Formula 1A, and
the electron acceptor group is selected from groups represented by Formula 1B: 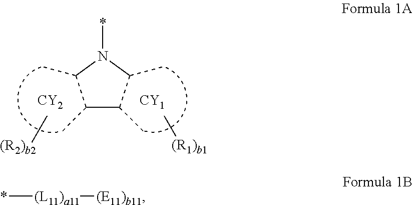
wherein CY₁ and CY₂ in Formula 1A are each independently selected from a benzene group, a naphthalene group, a carbazole group, a fluorene group, a dibenzofuran group, and a dibenzothiophene group,
R₁ and R₂ in Formula 1A are each independently selected from:
hydrogen, deuterium, a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, and a π electron-depleted nitrogen-free C₂-C₆₀ heterocyclic group; and
a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, and a π electron-depleted nitrogen-free C₂-C₆₀ heterocyclic group, each substituted with at least one selected from deuterium, a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, a (C₁-C₁₀ alkyl)C₅-C₆₀ carbocyclic group, a di(C₁-C₁₀ alkyl)C₅-C₆₀ carbocyclic group, a (phenyl)C₅-C₆₀ carbocyclic group, a di(phenyl)C₅-C₆₀ carbocyclic group, a (biphenyl)C₅-C₆₀ carbocyclic group, a di(biphenyl)C₅-C₆₀ carbocyclic group, a π electron-depleted nitrogen-free C₂-C₆₀ heterocyclic group, a (C₁-C₁₀ alkyl) π electron-depleted nitrogen-free C₂-C₆₀ heterocyclic group, a di(C₁-C₁₀ alkyl) π electron-depleted nitrogen-free C₂-C₆₀ heterocyclic group, a (phenyl) π electron-depleted nitrogen-free C₂-C₆₀ heterocyclic group, a di(phenyl) π electron-depleted nitrogen-free C₂-C₆₀ heterocyclic group, a (biphenyl) π electron-depleted nitrogen-free C₂-C₆₀ heterocyclic group, and a di(biphenyl) π electron-depleted nitrogen-free C₂-C₆₀ heterocyclic group,
b1 and b2 in Formula 1A are each independently an integer from 0 to 6,
“*” in Formula 1A indicates a binding site to a neighboring atom, provided that “*” in Formula 1A does not indicate a binding site to an electron acceptor group,
CY₁ and CY₂ in Formula 1A are optionally additionally chemically bonded to at least one of an electron donor group and an electron acceptor group,
L₁₁ in Formula 1B is selected from:
a single bond, cyclopentane group, a cyclohexane group, a cycloheptane group, a cyclooctane group, a cyclopentene group, a cyclohexene group, a cycloheptene group, a benzene group, a naphthalene group, a fluorene group, a phenanthrene group, an anthracene group, a fluoranthene group, a triphenylene group, a pyrene group, a chrysene group, a pyrrole group, a thiophene group, a furan group, an imidazole group, a pyrazole group, a thiazole group, an isothiazole group, an oxazole group, an isoxazole group, a pyridine group, a pyrazine group, a pyrimidine group, a pyridazine group, an isoindole group, an indole group, an indazole group, a purine group, a quinoline group, an isoquinoline group, a benzoquinoline group, a quinoxaline group, a quinazoline group, a cinnoline group, a phenanthroline group, a benzimidazole group, a benzofuran group, a benzothiophene group, a benzoxazole group, an isobenzoxazole group, a triazole group, a tetrazole group, an oxadiazole group, a triazine group, a dibenzofuran group, a dibenzothiophene group, a benzocarbazole group, a dibenzocarbazole group, an imidazopyridine group, an imidazopyrimidine group, an azaindole group, an azaindene group, an azabenzofuran group, an azabenzothiophene group, an azacarbazole group, an azafluorene group, an azadibenzofuran group, and an azadibenzothiophene group; and
a cyclopentane group, a cyclohexane group, a cycloheptane group, a cyclooctane group, a cyclopentene group, a cyclohexene group, a cycloheptene group, a benzene group, a naphthalene group, a fluorene group, a phenanthrene group, an anthracene group, a fluoranthene group, a triphenylene group, a pyrene group, a chrysene group, a pyrrole group, a thiophene group, a furan group, an imidazole group, a pyrazole group, a thiazole group, an isothiazole group, an oxazole group, an isoxazole group, a pyridine group, a pyrazine group, a pyrimidine group, a pyridazine group, an isoindole group, an indole group, an indazole group, a purine group, a quinoline group, an isoquinoline group, a benzoquinoline group, a quinoxaline group, a quinazoline group, a cinnoline group, a phenanthroline group, a benzimidazole group, a benzofuran group, a benzothiophene group, a benzoxazole group, an isobenzoxazole group, a triazole group, a tetrazole group, an oxadiazole group, a triazine group, a dibenzofuran group, a dibenzothiophene group, a benzocarbazole group, a dibenzocarbazole group, an imidazopyridine group, an imidazopyrimidine group, an azaindole group, an azaindene group, an azabenzofuran group, an azabenzothiophene group, an azacarbazole group, an azafluorene group, an azadibenzofuran group, and an azadibenzothiophene group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a (C₁-C₂₀ alkyl)phenyl group, a di(C₁-C₂₀ alkyl)phenyl group, a (C₆-C₂₀ aryl)phenyl group, a di(C₆-C₂₀ aryl)phenyl group, a (C₃-C₂₀ heteroaryl)phenyl group, a di(C₃-C₂₀ heteroaryl)phenyl group, a pyridinyl group, a (C₁-C₂₀ alkyl)pyridinyl group, a di(C₁-C₂₀ alkyl)pyridinyl group, a (C₆-C₂₀ aryl)pyridinyl group, a di(C₆-C₂₀ aryl)pyridinyl group, a (C₃-C₂₀ heteroaryl)pyridinyl group, a di(C₃-C₂₀ heteroaryl)pyridinyl group, a pyrimidinyl group, a (C₁-C₂₀ alkyl)pyrimidinyl group, a di(C₁-C₂₀ alkyl)pyrimidinyl group, a (C₆-C₂₀ aryl)pyrimidinyl group, a di(C₆-C₂₀ aryl)pyrimidinyl group, a (C₃-C₂₀ heteroaryl)pyrimidinyl group, a di(C₃-C₂₀ heteroaryl)pyrimidinyl group, a triazinyl group, a (C₁-C₂₀ alkyl)triazinyl group, a di(C₁-C₂₀ alkyl)triazinyl group, a (C₆-C₂₀ aryl)triazinyl group, a di(C₆-C₂₀ aryl)triazinyl group, a (C₃-C₂₀ heteroaryl)triazinyl group, and a di(C₃-C₂₀ heteroaryl)triazinyl group,
a11 in Formula 1B is an integer from 1 to 3,
E₁₁ in Formula 1B is selected from:
—F, —CFH₂, —CF₂H, —CF₃, and —CN;
a C₁-C₆₀ alkyl group or a C₁-C₆₀ alkoxy group, substituted with at least one selected from —F, —CFH₂, —CF₂H, —CF₃, and —CN; and
a substituted or unsubstituted π electron-depleted nitrogen-containing C₂-C₆₀ heterocyclic group,
b11 in Formula 1B is an integer from 1 to 5,
a bond between L₁₁ and E₁₁ in Formula 1B is a carbon-carbon single bond or a carbon-fluorine single bond,
“*” in Formula 1B indicates a binding site to a neighboring carbon,
at least one substituent of the substituted π electron-depleted nitrogen-containing C₂-C₆₀ heterocyclic group is selected from:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and
—N(Q₃₁)(Q₃₂), —Si(Q₃₃)(Q₃₄)(Q₃₅), —B(Q₃₆)(Q₃₇), and —P(═O)(Q₃₈)(Q₃₉); and
Q₁₁ to Q₁₉, Q₂₁ to Q₂₉, and Q₃₁ to Q₃₉ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one of a C₁-C₆₀ alkyl group and a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group.

2. The organic light-emitting device of claim 1, wherein
n1 and n2 are each independently 1, 2, or 3, and
at least one of CY₁ and CY₂ in Formula 1A is a benzene group.

3. The organic light-emitting device of claim 1, wherein
R₁ and R₂ are each independently selected from:
hydrogen, deuterium, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a furanyl group, a thiophenyl group, an indolyl group, a benzofuranyl group, a benzothiophenyl group, a carbazolyl group, a dibenzofuranyl group, a dibenzothiophenyl group, a benzocarbazolyl group, a naphtho benzofuranyl group, a naphtho benzothiophenyl group, a dibenzocarbazolyl group, a dinaphthofuranyl group, a dinaphthothiophenyl group, an indolofluorenyl group, an indolocarbazolyl group, an indolodibenzofuranyl group, and an indolodibenzothiophenyl group; and
a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a furanyl group, a thiophenyl group, an indolyl group, a benzofuranyl group, a benzothiophenyl group, a carbazolyl group, a dibenzofuranyl group, a dibenzothiophenyl group, a benzocarbazolyl group, a naphtho benzofuranyl group, a naphtho benzothiophenyl group, a dibenzocarbazolyl group, a dinaphthofuranyl group, a dinaphthothiophenyl group, an indolofluorenyl group, an indolocarbazolyl group, an indolodibenzofuranyl group, and an indolodibenzothiophenyl group, each substituted with at least one selected from deuterium, a C₁-C₂₀ alkyl group, a C₁-C₂₀ alkoxy group, a phenyl group, a biphenyl group, a terphenyl group, a naphthyl group, a fluorenyl group, a dimethylfluorenyl group, a diphenylfluorenyl group, a carbazolyl group, a phenylcarbazolyl group, a biphenylcarbazolyl group, a dibenzofuranyl group, and a dibenzothiophenyl group,
b1 and b2 are each independently 0, 1, or 2, and
the sum of b1 and b2 is 0, 1, or 2.

4. The organic light-emitting device of claim 1, wherein
R₁ and R₂ are each independently selected from hydrogen, deuterium, a C₁-C₂₀ alkyl group, a C₁-C₂₀ alkoxy group, and groups represented by Formulae 2-1 to 2-26: 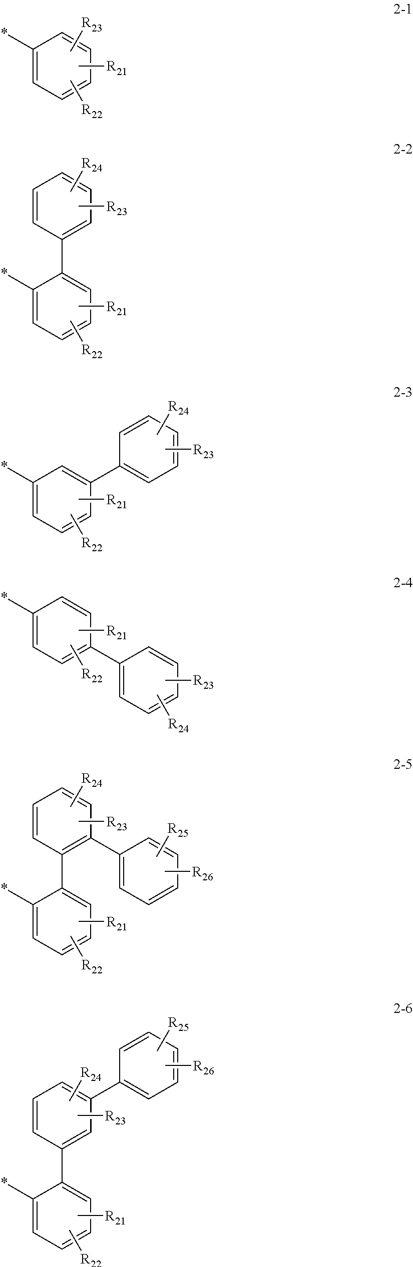 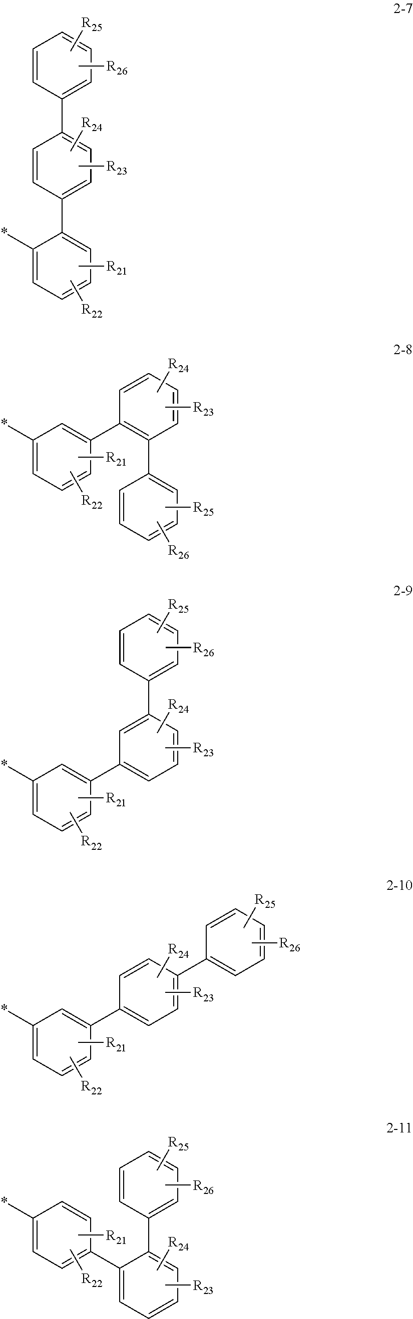 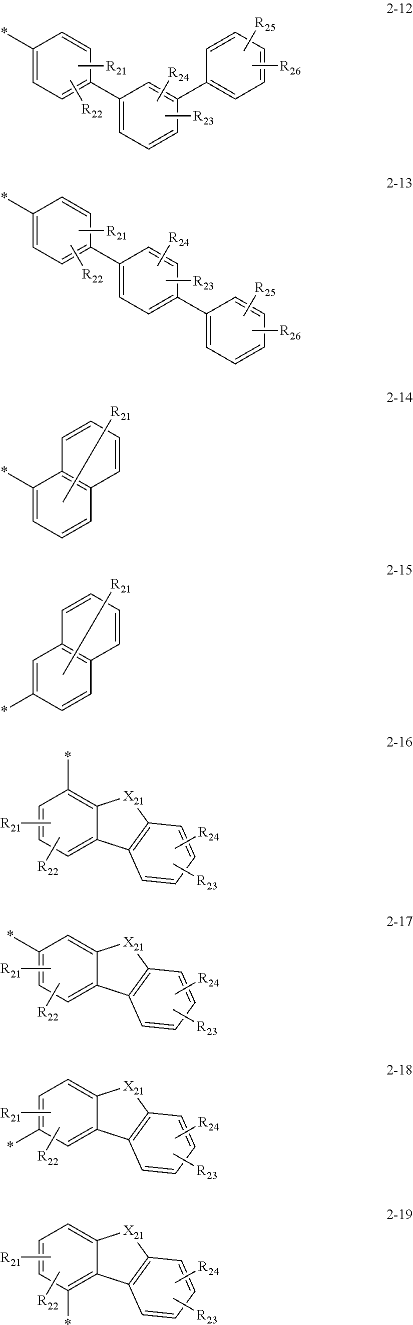 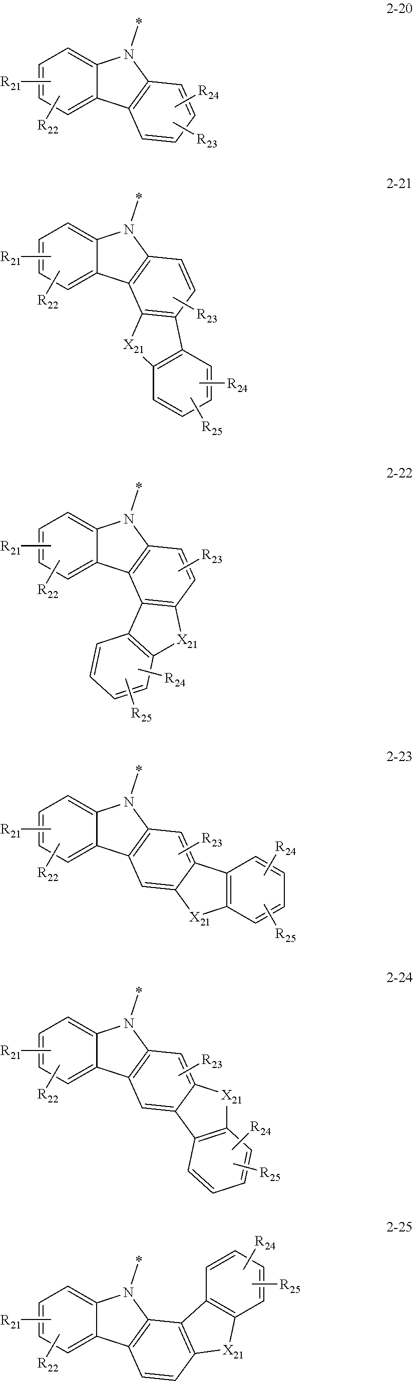 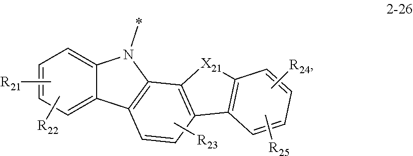
wherein, in Formulae 2-1 to 2-26,
X₂₁ is C(R₂₇)(R₂₈), N(R₂₉), O, or S,
R₂₁ to R₂₉ are each independently selected from hydrogen, deuterium, a C₁-C₂₀ alkyl group, a C₁-C₂₀ alkoxy group, a phenyl group, a biphenyl group, a terphenyl group, a naphthyl group, a fluorenyl group, a dimethylfluorenyl group, a diphenylfluorenyl group, a carbazolyl group, a phenylcarbazolyl group, a biphenylcarbazolyl group, a dibenzofuranyl group, and a dibenzothiophenyl group, and
“*” indicates a binding site to a neighboring atom.

5. The organic light-emitting device of claim 1, wherein
L₁₁ is selected from:
a single bond, a benzene group, a naphthalene group, a fluorene group, a pyridine group, a pyrazine group, a pyrimidine group, a pyridazine group, a quinoline group, an isoquinoline group, a benzoquinoline group, a quinoxaline group, a quinazoline group, and a triazine group; and
a benzene group, a naphthalene group, a fluorene group, a pyridine group, a pyrazine group, a pyrimidine group, a pyridazine group, a quinoline group, an isoquinoline group, a benzoquinoline group, a quinoxaline group, a quinazoline group, and a triazine group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a (C₁-C₂₀ alkyl)phenyl group, a di(C₁-C₂₀ alkyl)phenyl group, a (C₆-C₂₀ aryl)phenyl group, a di(C₆-C₂₀ aryl)phenyl group, a (C₃-C₂₀ heteroaryl)phenyl group, a di(C₃-C₂₀ heteroaryl)phenyl group, a pyridinyl group, a (C₁-C₂₀ alkyl)pyridinyl group, a di(C₁-C₂₀ alkyl)pyridinyl group, a (C₆-C₂₀ aryl)pyridinyl group, a di(C₆-C₂₀ aryl)pyridinyl group, a (C₃-C₂₀ heteroaryl)pyridinyl group, a di(C₃-C₂₀ heteroaryl)pyridinyl group, a pyrimidinyl group, a (C₁-C₂₀ alkyl)pyrimidinyl group, a di(C₁-C₂₀ alkyl)pyrimidinyl group, a (C₆-C₂₀ aryl)pyrimidinyl group, a di(C₆-C₂₀ aryl)pyrimidinyl group, a (C₃-C₂₀ heteroaryl)pyrimidinyl group, a di(C₃-C₂₀ heteroaryl)pyrimidinyl group, a triazinyl group, a (C₁-C₂₀ alkyl)triazinyl group, a di(C₁-C₂₀ alkyl)triazinyl group, a (C₆-C₂₀ aryl)triazinyl group, a di(C₆-C₂₀ aryl)triazinyl group, a (C₃-C₂₀ heteroaryl)triazinyl group, and a di(C₃-C₂₀ heteroaryl)triazinyl group, and
a11 is 1, 2, or 3.

6. The organic light-emitting device of claim 1, wherein
E₁₁ is selected from:
—F, —CFH₂, —CF₂H, —CF₃, and —CN;
a C₁-C₂₀ alkyl group substituted with at least one selected from —F, —CFH₂, —CF₂H, —CF₃, and —CN; and
groups represented by Formulae 3-1 to 3-14: 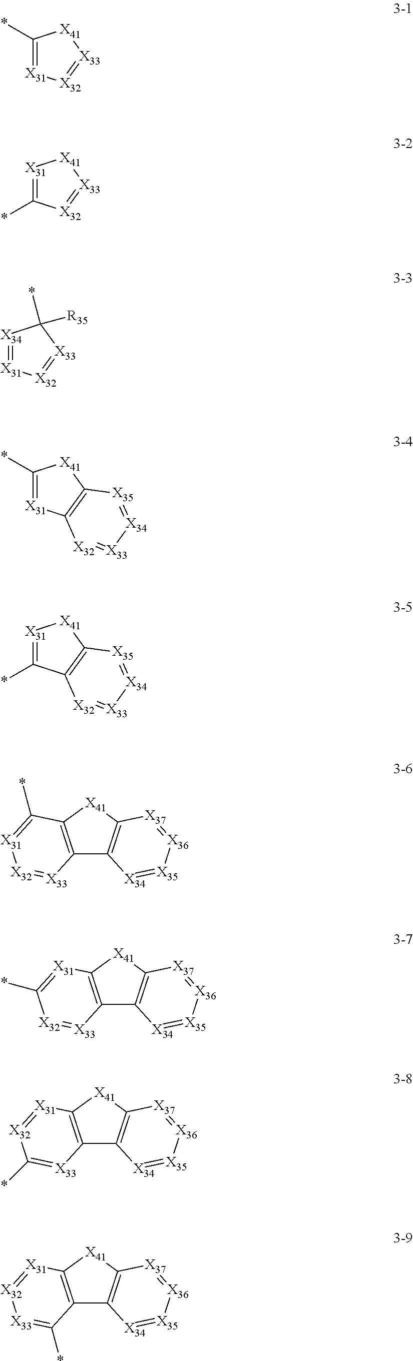 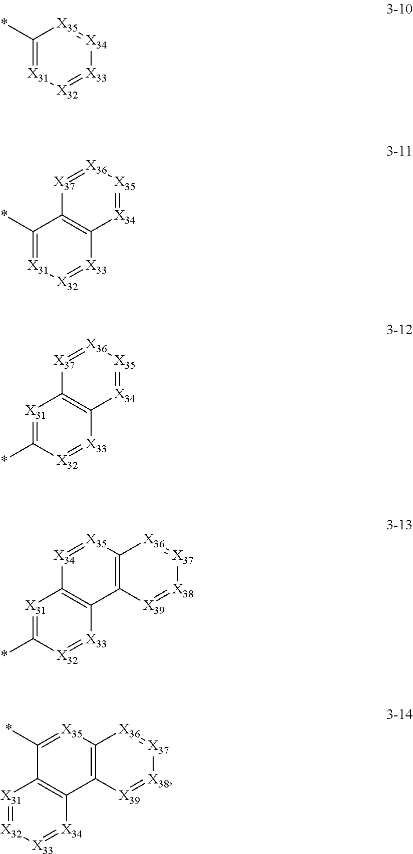
wherein, in Formulae 3-1 to 3-14, X₃₁ is N or C(R₃₁), X₃₂ is N or C(R₃₂), X₃₃ is N or C(R₃₃), X₃₄ is N or C(R₃₄), X₃₅ is N or C(R₃₅), X₃₆ is N or C(R₃₆), X₃₇ is N or C(R₃₇), X₃₈ is N or C(R₃₈), and X₃₉ is N or C(R₃₉),
X₄₁ in Formulae 3-1, 3-2, and 3-4 to 3-9 is N(R₄₁), C(R₄₂)(R₄₃), O, or S,
at least one of X₃₁ to X₃₃ in Formulae 3-1 and 3-2 is N, at least one of X₃₁ to X₃₄ in Formula 3-3 is N, at least one of X₃₁ to X₃₅ in Formulae 3-4, 3-5, and 3-10 is N, at least one of X₃₁ to X₃₇ in Formulae 3-6 to 3-9, 3-11, and 3-12 is N, and at least one of X₃₁ to X₃₉ in Formulae 3-13 and 3-14 is N,
R₃₁ to R₃₉ and R₄₁ to R₄₃ are each independently selected from hydrogen, deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a (C₁-C₂₀ alkyl)phenyl group, a di(C₁-C₂₀ alkyl)phenyl group, a (C₆-C₂₀ aryl)phenyl group, a di(C₆-C₂₀ aryl)phenyl group, a (C₃-C₂₀ heteroaryl)phenyl group, a di(C₃-C₂₀ heteroaryl)phenyl group, a pyridinyl group, a (C₁-C₂₀ alkyl)pyridinyl group, a di(C₁-C₂₀ alkyl)pyridinyl group, a (C₆-C₂₀ aryl)pyridinyl group, a di(C₆-C₂₀ aryl)pyridinyl group, a (C₃-C₂₀ heteroaryl)pyridinyl group, a di(C₃-C₂₀ heteroaryl)pyridinyl group, a pyrimidinyl group, a (C₁-C₂₀ alkyl)pyrimidinyl group, a di(C₁-C₂₀ alkyl)pyrimidinyl group, a (C₆-C₂₀ aryl)pyrimidinyl group, a di(C₆-C₂₀ aryl)pyrimidinyl group, a (C₃-C₂₀ heteroaryl)pyrimidinyl group, a di(C₃-C₂₀ heteroaryl)pyrimidinyl group, a triazinyl group, a (C₁-C₂₀ alkyl)triazinyl group, a di(C₁-C₂₀ alkyl)triazinyl group, a (C₆-C₂₀ aryl)triazinyl group, a di(C₆-C₂₀ aryl)triazinyl group, a (C₃-C₂₀ heteroaryl)triazinyl group, and a di(C₃-C₂₀ heteroaryl)triazinyl group, and
“*” indicates a binding site to a neighboring atom.

7. The organic light-emitting device of claim 1, wherein
E₁₁ is selected from:
—CN;
a C₁-C₂₀ alkyl group substituted with at least one —CN; and
groups represented by Formulae 3-4(1) to 3-4(4), 3-5(1) to 3-5(4), 3-6(1), 3-7(1), 3-8(1), 3-9(1), 3-10(1) to 3-10(8), 3-11(1) to 3-11(23), and 3-12(1) to 3-12(23): 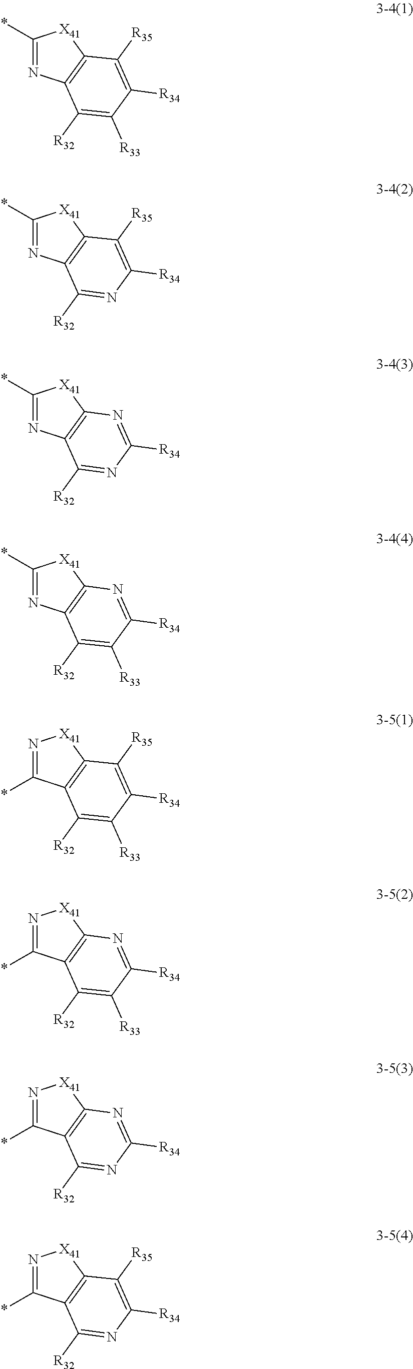 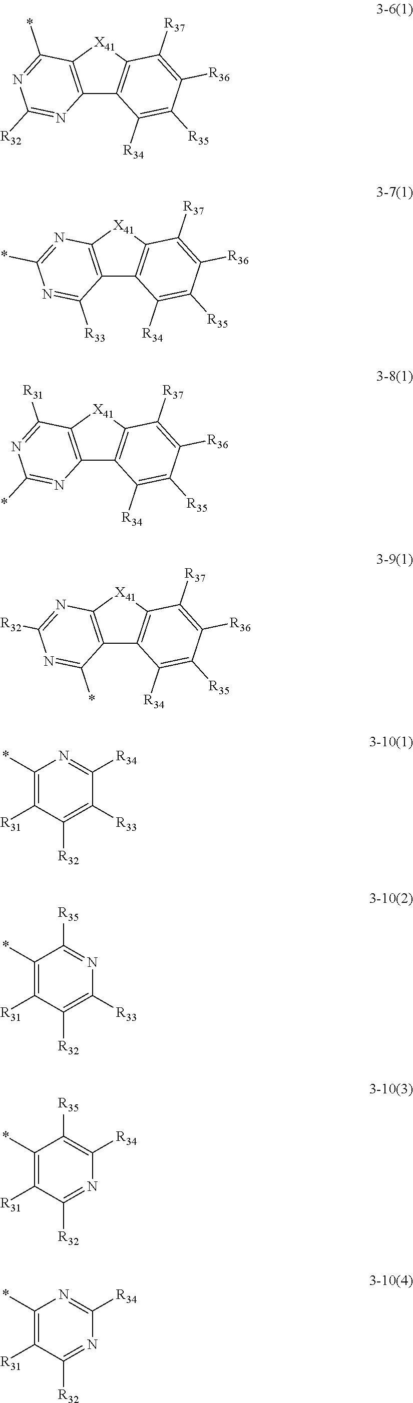 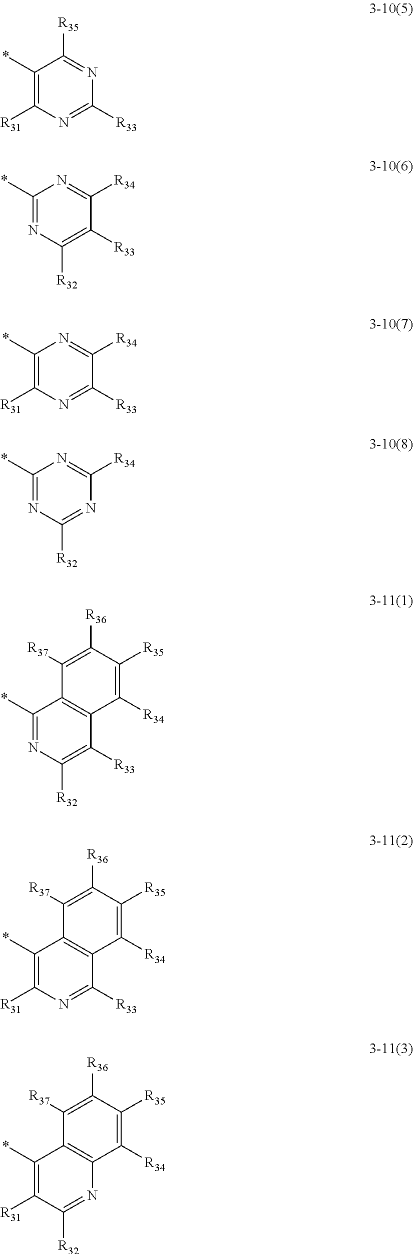 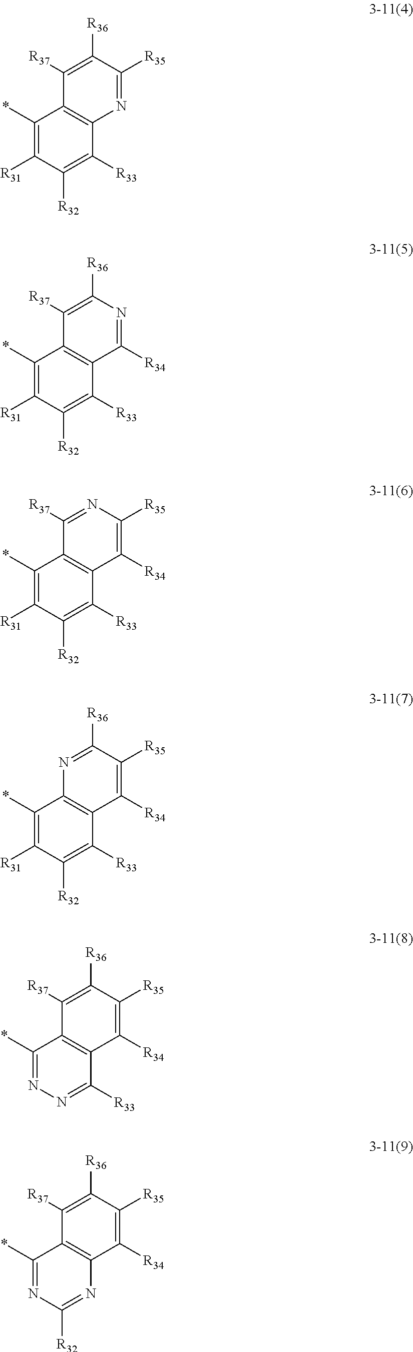 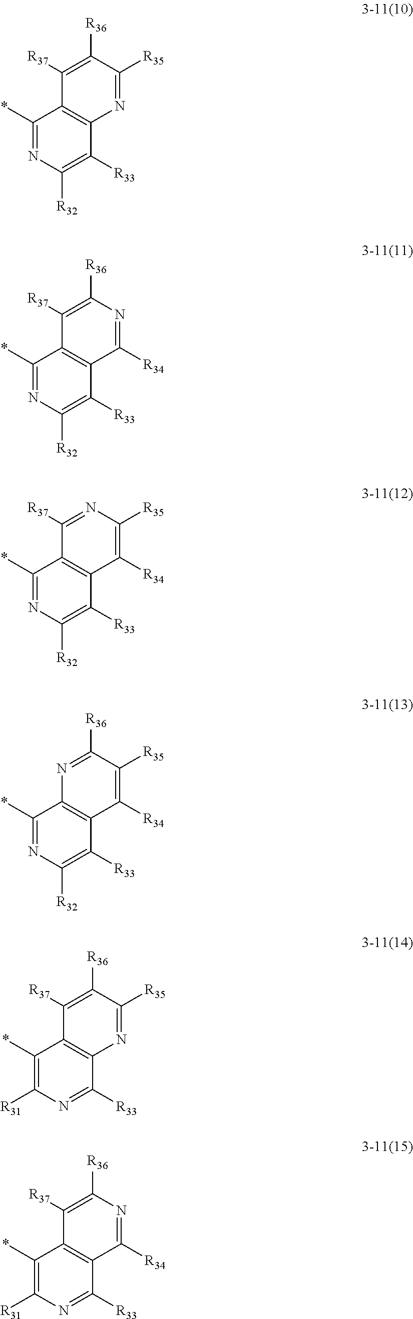 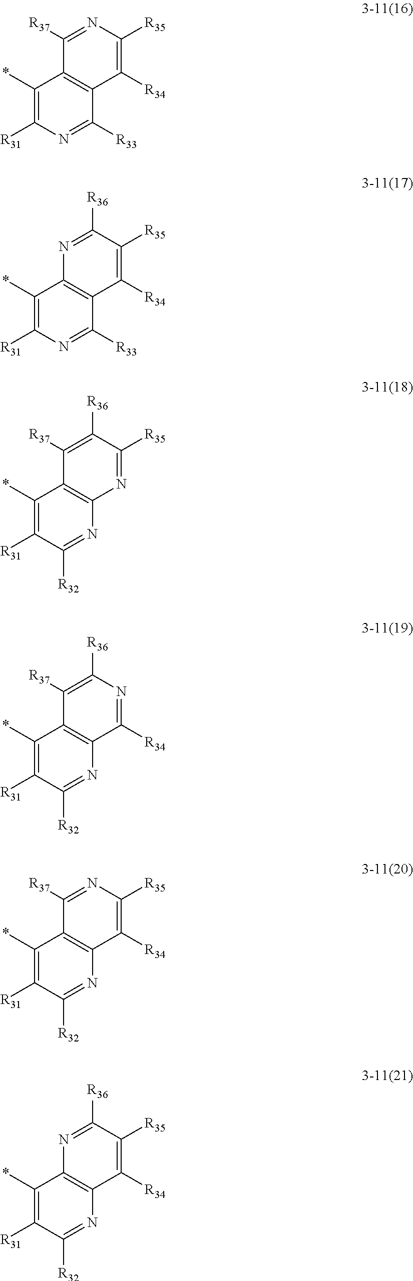 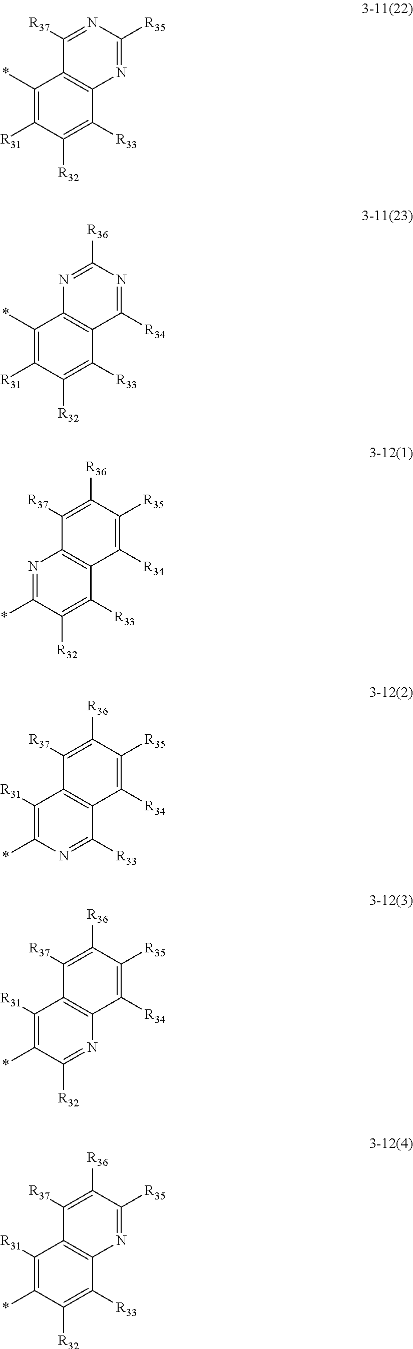 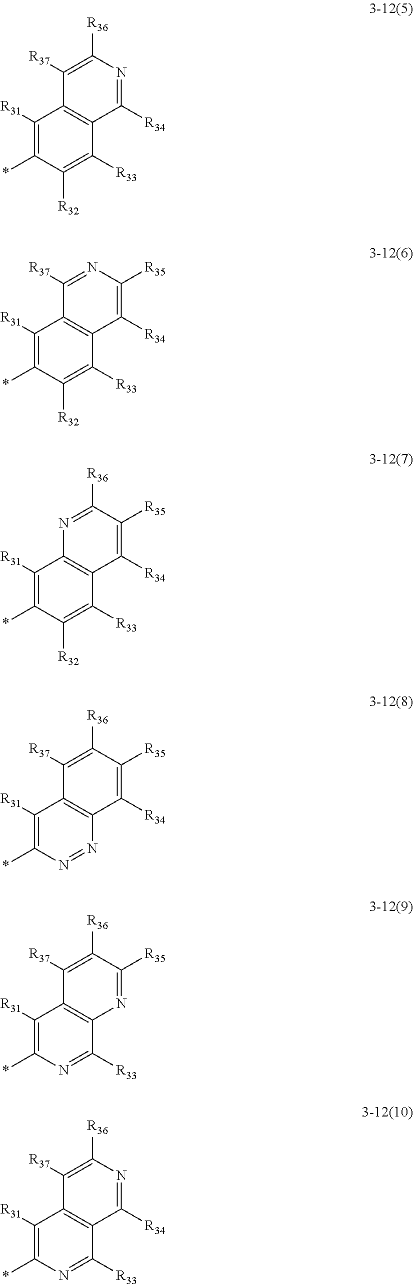 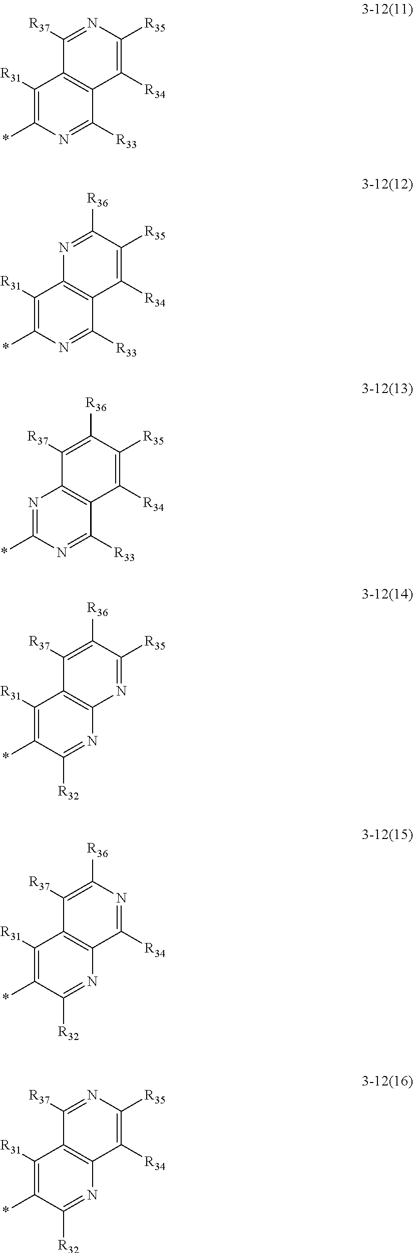 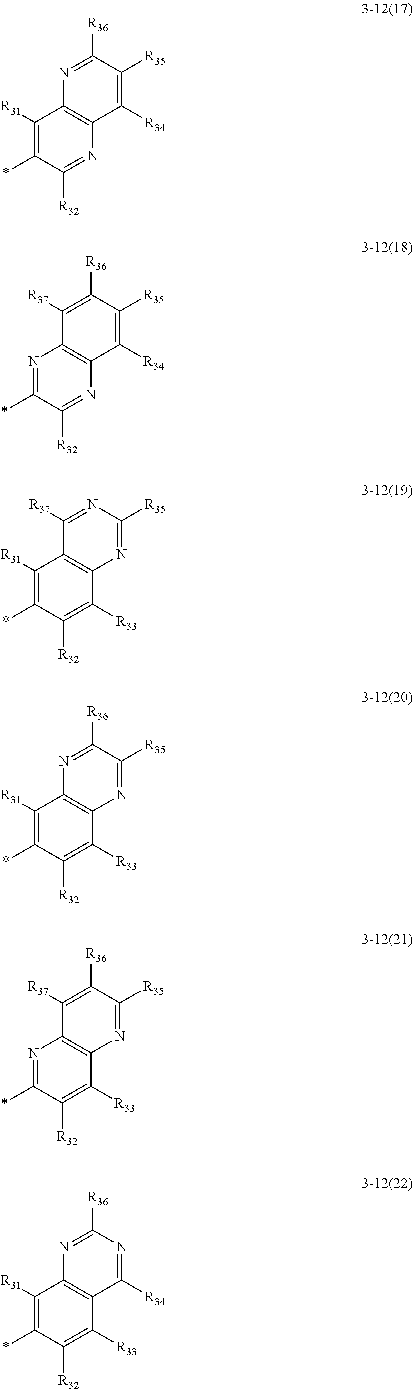 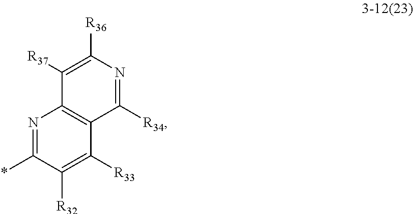
wherein, in Formulae 3-4(1) to 3-4(4), 3-5(1) to 3-5(4), 3-6(1), 3-7(1), 3-8(1), 3-9(1), 3-10(1) to 3-10(8), 3-11(1) to 3-11(23), and 3-12(1) to 3-12(23), X₄₁ is N(R₄₁), C(R₄₂)(R₄₃), O, or S,
R₃₁ to R₃₇ and R₄₁ to R₄₃ are each independently selected from hydrogen, deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a (C₁-C₁₀ alkyl)phenyl group, a di(C₁-C₁₀ alkyl)phenyl group, a biphenyl group, a di(phenyl)phenyl group, a (pyridinyl)phenyl group, a di(pyridinyl)phenyl group, a (pyrimidinyl)phenyl group, a di(pyrimidinyl)phenyl group, a (triazinyl)phenyl group, a di(triazinyl)phenyl group, a pyridinyl group, a (C₁-C₁₀ alkyl)pyridinyl group, a di(C₁-C₁₀ alkyl)pyridinyl group, a (phenyl)pyridinyl group, a di(phenyl)pyridinyl group, a (pyridinyl)pyridinyl group, a di(pyridinyl)pyridinyl group, a (pyrimidinyl)pyridinyl group, a di(pyrimidinyl)pyridinyl group, a (triazinyl)pyridinyl group, a di(triazinyl)pyridinyl group, a triazinyl group, a (C₁-C₁₀ alkyl)triazinyl group, a di(C₁-C₁₀ alkyl)triazinyl group, a (phenyl)triazinyl group, a di(phenyl)triazinyl group, a (pyridinyl)triazinyl group, a di(pyridinyl)triazinyl group, a (pyrimidinyl)triazinyl group, a di(pyrimidinyl)triazinyl group, a (triazinyl)triazinyl group, and a di(triazinyl)triazinyl group, and
“*” indicates a binding site to a neighboring atom.

8. The organic light-emitting device of claim 1, wherein
R₁ and R₂ are each independently selected from hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, a di(phenyl)phenyl group, a fluorenyl group, a (C₁-C₁₀ alkyl)fluorenyl group, a di(C₁-C₁₀ alkyl)fluorenyl group, a (phenyl)fluorenyl group, a di(phenyl)fluorenyl group, a carbazolyl group, a (C₁-C₁₀ alkyl)carbazolyl group, a di(C₁-C₁₀ alkyl)carbazolyl group, a (phenyl)carbazolyl group, a di(phenyl)carbazolyl group, a dibenzofuranyl group, a (C₁-C₁₀ alkyl)dibenzofuranyl group, a di(C₁-C₁₀ alkyl)dibenzofuranyl group, a (phenyl)dibenzofuranyl group, a di(phenyl)dibenzofuranyl group, a dibenzothiophenyl group, a (C₁-C₁₀ alkyl)dibenzothiophenyl group, a di(C₁-C₁₀ alkyl)dibenzothiophenyl group, a (phenyl)dibenzothiophenyl group, a di(phenyl)dibenzothiophenyl group, a indolofluorenyl group, a (C₁-C₁₀ alkyl)indolofluorenyl group, a di(C₁-C₁₀ alkyl)indolofluorenyl group, a (phenyl)indolofluorenyl group, a di(phenyl)indolofluorenyl group, an indolocarbazolyl group, a (C₁-C₁₀ alkyl)indolocarbazolyl group, a di(C₁-C₁₀ alkyl)indolocarbazolyl group, a (phenyl)indolocarbazolyl group, a di(phenyl)indolocarbazolyl group, an indolodibenzofuranyl group, a (C₁-C₁₀ alkyl)indolodibenzofuranyl group, a di(C₁-C₁₀ alkyl)indolodibenzofuranyl group, a (phenyl)indolodibenzofuranyl group, a di(phenyl)indolodibenzofuranyl group, an indolodibenzothiophenyl group, a (C₁-C₁₀ alkyl)indolodibenzothiophenyl group, a di(C₁-C₁₀ alkyl)indolodibenzothiophenyl group, a (phenyl)indolodibenzothiophenyl group, and a di(phenyl)indolodibenzothiophenyl group,
b1 and b2 are each independently 0, 1, or 2, and
the electron acceptor group is selected from —CN and groups represented by Formulae 1B-1 to 1B-30: 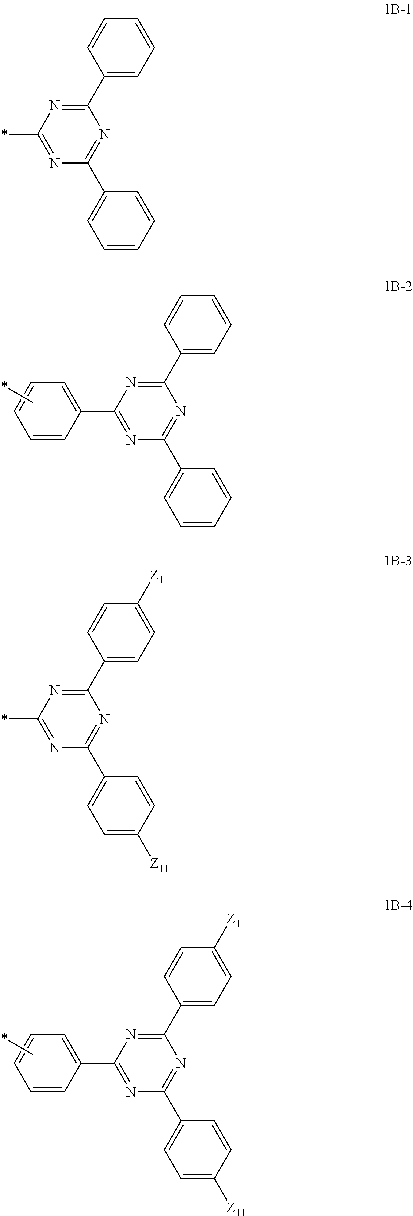 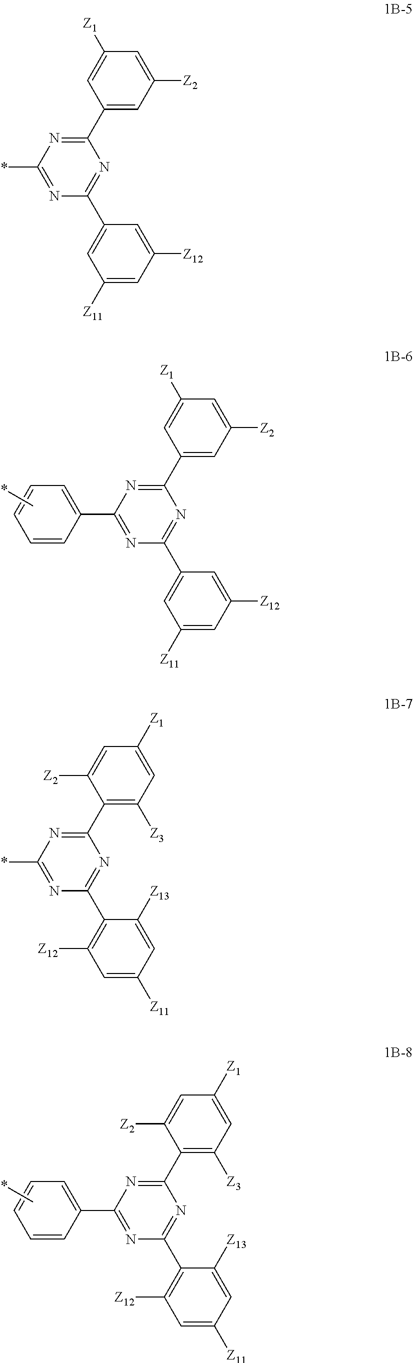 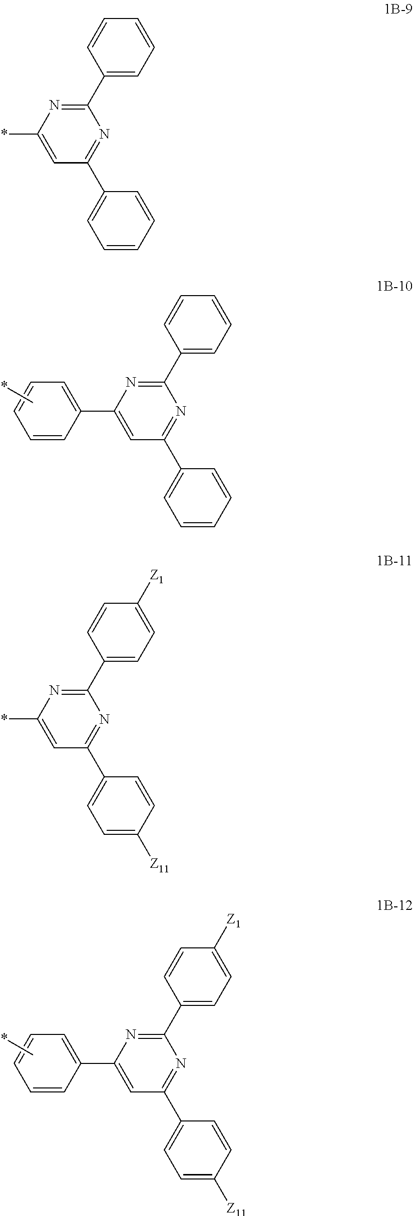 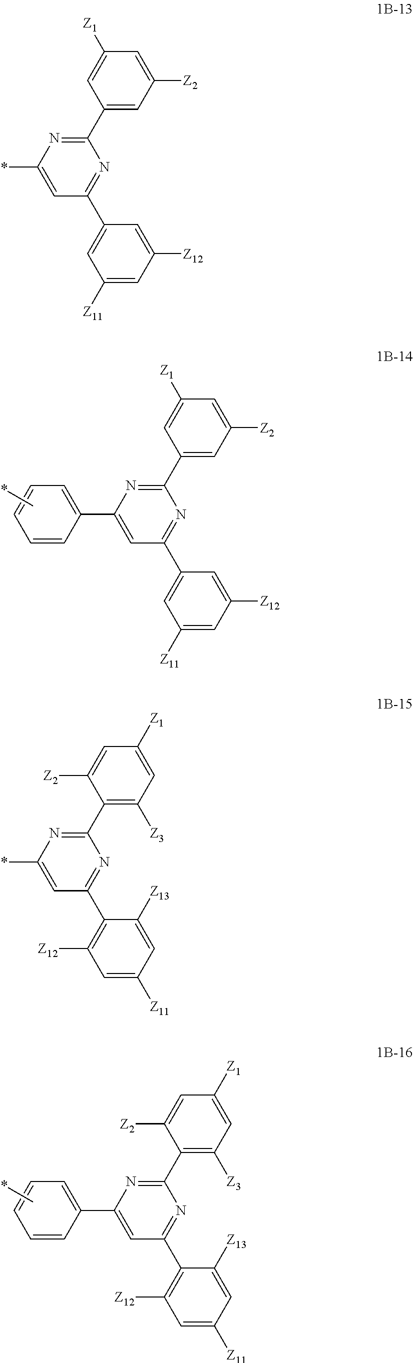 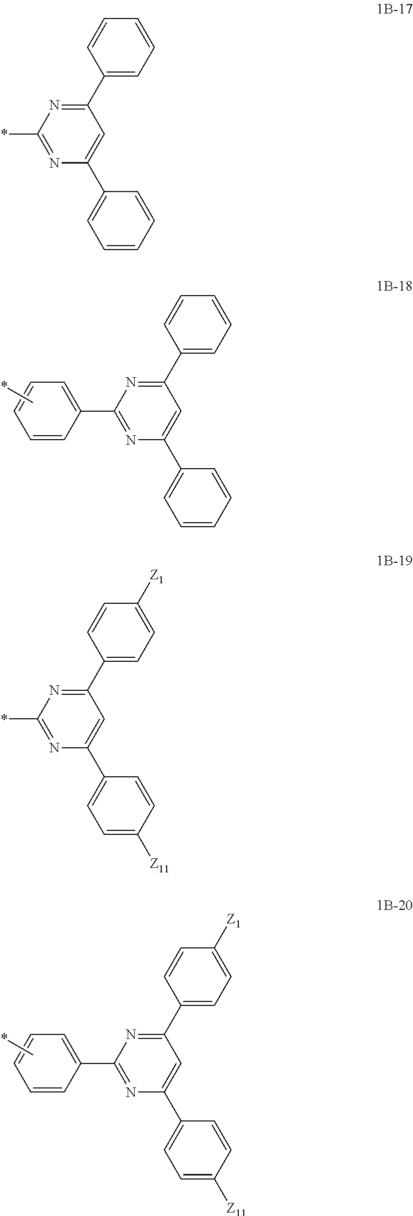 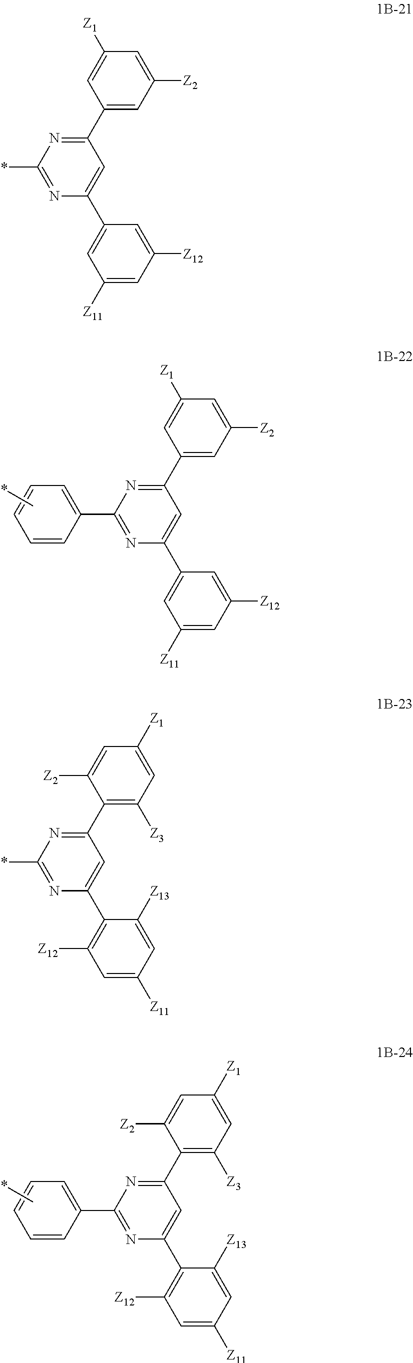 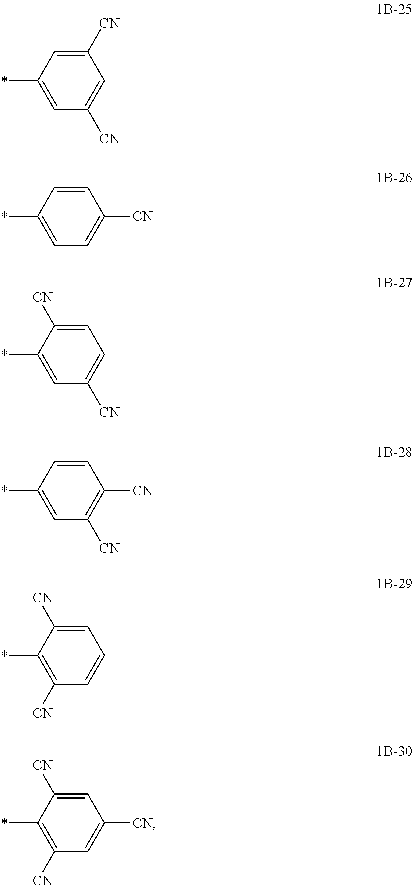
wherein, in Formulae 1B-1 to 1B-30,
Z₁ to Z₃ and Z₁₁ to Z₁₃ are each independently selected from a C₁-C₁₀ alkyl group and a phenyl group, and
“*” indicates a binding site to a neighboring atom.

9. The organic light-emitting device of claim 1, wherein
the fluorescent compound is represented by one of Formulae 10-1 to 10-6: 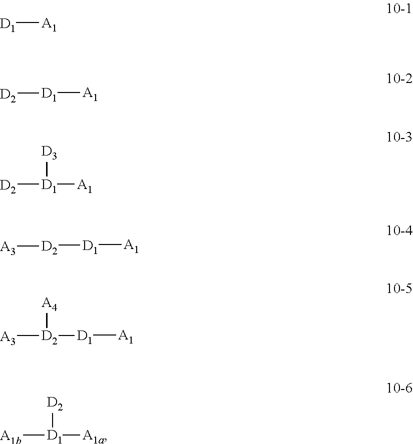
wherein, in Formulae 10-1 to 10-6,
D₁ to D₃ are each independently selected from electron donor groups represented by Formula 1A, and
A₁, A₁ₐ, A_{1b}, A₃, and A₄ are each independently selected from electron acceptor groups represented by Formula 1B.

10. The organic light-emitting device of claim 1, wherein
the fluorescent compound is represented by one of Formulae 9-1 to 9-9: 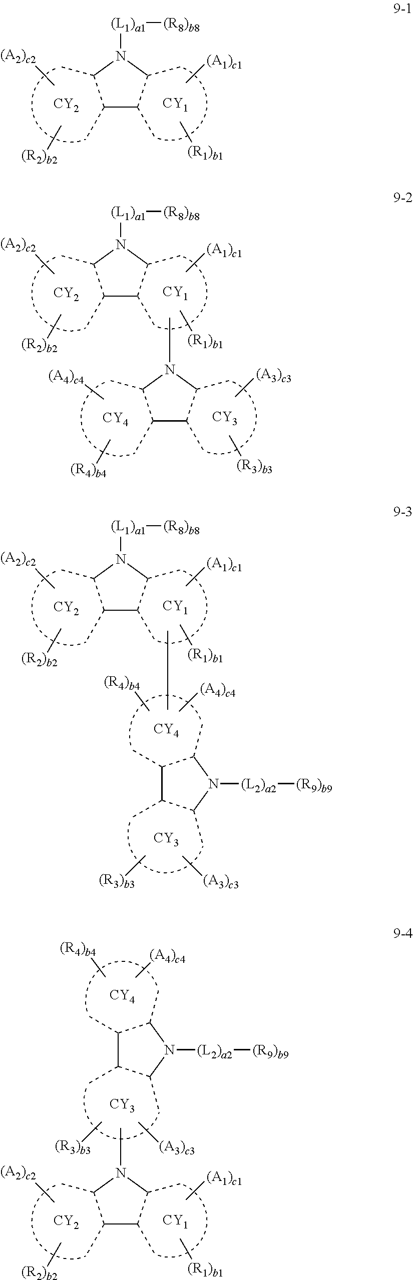 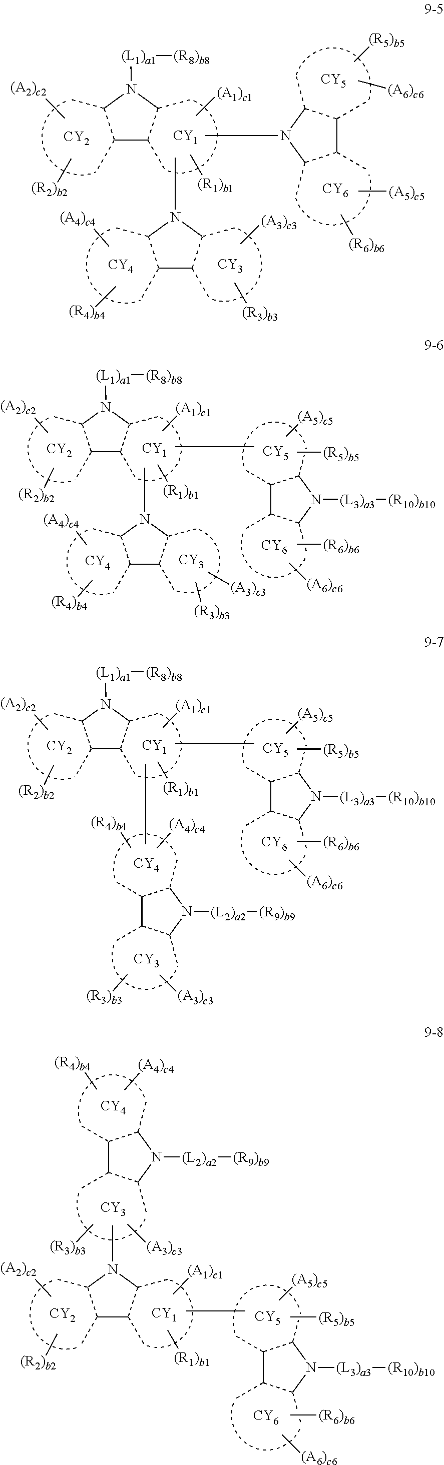 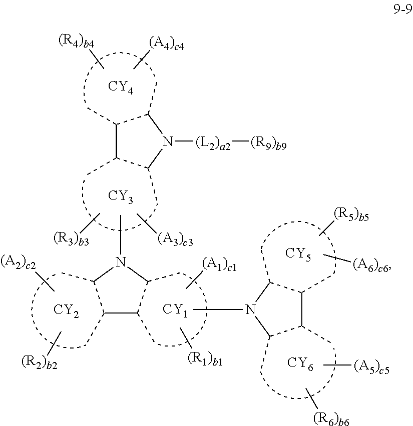
wherein, in Formulae 9-1 to 9-9,
CY₁, CY₂, R₁, R₂, b1, and b2 are the same as described in claim 1,
L₁ to L₃ are each independently selected from:
a single bond, a C₅-C₆₀ carbocyclic group, and a π electron-depleted nitrogen-free C₂-C₆₀ heterocyclic group; and
a C₅-C₆₀ carbocyclic group and a π electron-depleted nitrogen-free C₂-C₆₀ heterocyclic group, each substituted with at least one selected from deuterium, a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, a (C₁-C₁₀ alkyl)C₅-C₆₀ carbocyclic group, a di(C₁-C₁₀ alkyl)C₅-C₆₀ carbocyclic group, a (phenyl)C₅-C₆₀ carbocyclic group, a di(phenyl)C₅-C₆₀ carbocyclic group, a (biphenyl)C₅-C₆₀ carbocyclic group, a di(biphenyl)C₅-C₆₀ carbocyclic group, a π electron-depleted nitrogen-free C₂-C₆₀ heterocyclic group, a (C₁-C₁₀ alkyl) π electron-depleted nitrogen-free C₂-C₆₀ heterocyclic group, a di(C₁-C₁₀ alkyl) π electron-depleted nitrogen-free C₂-C₆₀ heterocyclic group, a (phenyl) π electron-depleted nitrogen-free C₂-C₆₀ heterocyclic group, a di(phenyl) π electron-depleted nitrogen-free C₂-C₆₀ heterocyclic group, a (biphenyl) π electron-depleted nitrogen-free C₂-C₆₀ heterocyclic group, and a di(biphenyl) π electron-depleted nitrogen-free C₂-C₆₀ heterocyclic group,
a1 to a3 are each independently an integer from 1 to 3,
R₈ to R₁₀ are the same as described in connection with R₁ in claim 1, provided that R₈ to R₁₀ are not hydrogen,
b8 to b10 are each independently an integer from 1 to 5,
R₁ to R₆ are the same as described in connection with R₁ in claim 1,
b1 to b6 are the same as described in connection with b1 in claim 1,
A₁ to A₆ are each independently selected from electron acceptor groups represented by Formula 1B,
c1 to c6 are each independently 0, 1, 2, or 3, provided that the sum of c1 and c2 in Formula 9-1 is one or more, the sum of c1 to c4 in Formulae 9-2 to 9-4 is one or more, and the sum of c1 to c6 in Formulae 9-5 to 9-9 is one or more, and
a bond between A₁ and CY₁, a bond between A₂ and CY₂, a bond between A₃ and CY₃, a bond between A₄ and CY₄, a bond between A₅ and CY₅, and a bond between A₆ and CY₆ are each a carbon-carbon single bond.

11. The organic light-emitting device of claim 10, wherein,
in Formulae 9-1 to 9-9, a moiety represented by 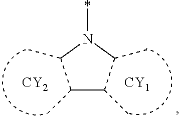
a moiety represented by 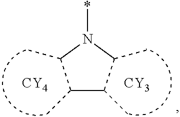
and a moiety represented by 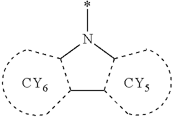
are each independently derived from groups represented by Formulae 8-1 to 8-7: 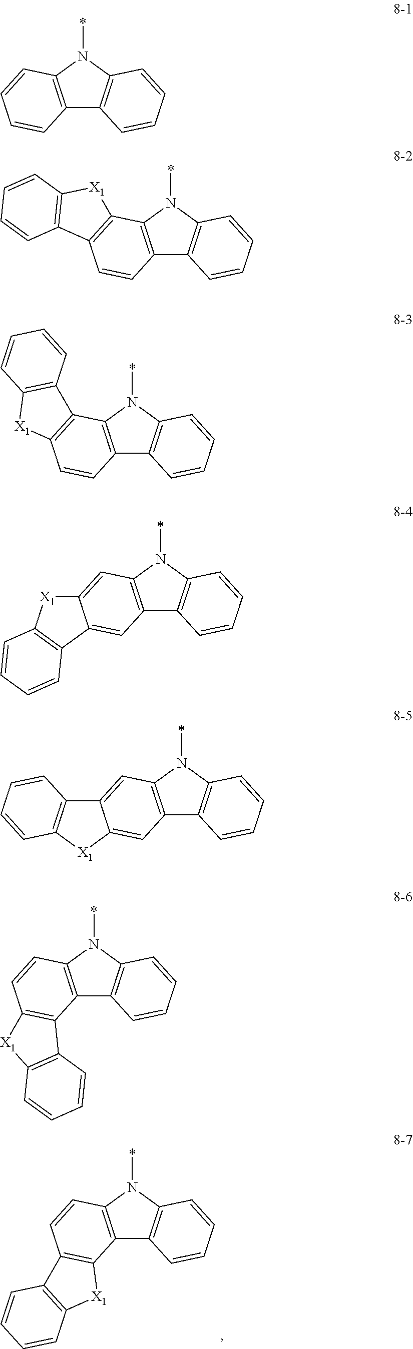
wherein, in Formulae 8-2 to 8-7, X₁ is N(R′), C(R′)(R″), O, or S, R′ and R″ are the same as described in connection with R₁ in claim 1 and “*” indicates a binding site to a neighboring atom.

12. The organic light-emitting device of claim 10, wherein
1) in Formula 9-1, c1 is 1 or 2 and c2 is 0,
2) in Formulae 9-2 to 9-4, i) c1 is 1 or 2, c2 is 0, and the sum of c3 and c4 is 0, 1, or 2, or ii) c1 is 1, c2 is 1 or 2, and the sum of c3 and c4 is 0, 1, or 2, and
3) in Formulae 9-5 to 9-9, i) c1 is 1 or 2, c2 is 0, and the sum of c3, c4, c5, and c6 is 1 or 2, or ii) c1 is 0, c2 is 1 or 2, and the sum of c3, c4, c5, and c6 is 0, 1, or 2.

13. The organic light-emitting device of claim 1, wherein
the fluorescent compound is represented by one selected from Formulae 1-1 to 1-7: 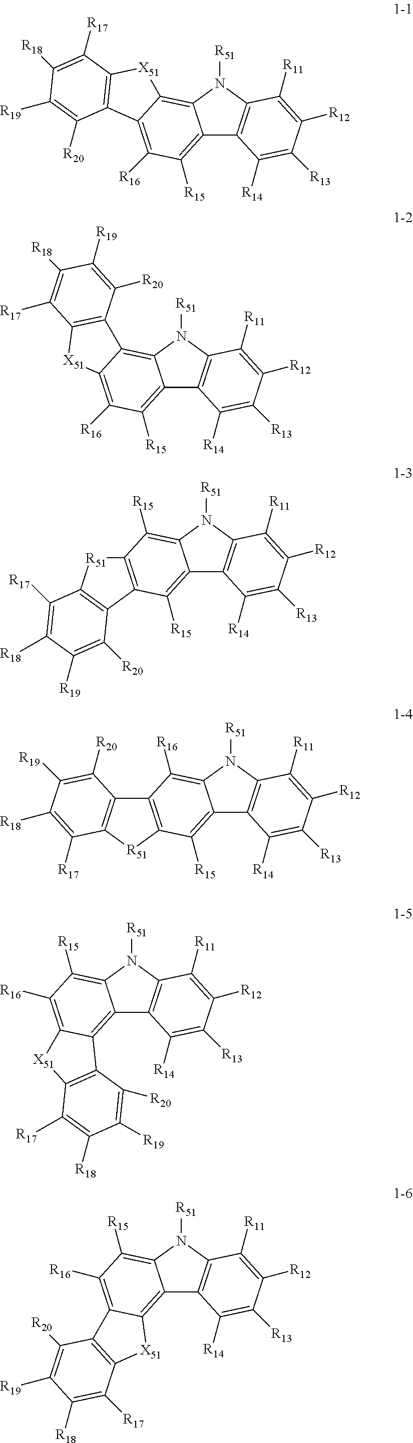 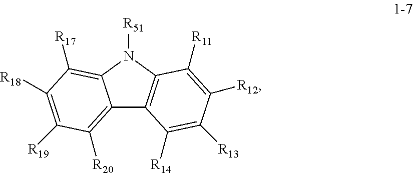
wherein, in Formulae 1-1 to 1-7,
X₅₁ is C(R₅₂)(R₅₃), N(R₅₂), O or S, and
1) i) R₁₁ is an electron acceptor group represented by Formula 1B, and ii) one selected from R₁₂ to R₂₀ and R₅₁ to R₅₃ is a group represented by one of Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
2) i) R₁₂ is an electron acceptor group represented by Formula 1B, and ii) one selected from R₁₁, R₁₃ to R₂₀, and R₅₁ to R₅₃ is a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
3) i) R₁₃ is an electron acceptor group represented by Formula 1B, and ii) one selected from R₁₁, R₁₂, R₁₄ to R₂₀, and R₅₁ to R₅₃ is a group represented by Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
4) i) R₁₄ is an electron acceptor group represented by Formula 1B, and ii) one selected from R₁₁ to R₁₃, R₁₅ to R₂₀, and R₅₁ to R₅₃ is a group a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
5) i) R₁₅ is an electron acceptor group represented by Formula 1B, and ii) one selected from R₁₁ to R₁₄, R₁₆ to R₂₀, and R₅₁ to R₅₃ is a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
6) i) R₁₆ is an electron acceptor group represented by Formula 1B, and ii) one selected from R₁₁ to R₁₅, R₁₇ to R₂₀, and R₅₁ to R₅₃ is a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
7) i) R₁₂ is an electron acceptor group represented by Formula 1B, ii) R₁₁ and R₁₃ are each independently a group represented by one selected from Formulae 6-1 to 6-7, and iii) R₁₄ to R₂₀ and R₅₁ to R₅₃ are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
8) i) R₁₃ is an electron acceptor group represented by Formula 1B, ii) R₁₂ and R₁₄ are each independently a group represented by one selected from Formulae 6-1 to 6-7, and iii) R₁₁, R₁₅ to R₂₀, and R₅₁ to R₅₃ are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
9) i) R₁₂ and R₁₃ are each independently an electron acceptor group represented by Formula 1B, and ii) one selected from R₁₁, R₁₄ to R₂₀, and R₅₁ to R₅₃ is a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
10) i) R₁₁ and R₁₂ are each independently an electron acceptor group represented by Formula 1B, and ii) one selected from R₁₃ to R₂₀ and R₅₁ to R₅₃ is a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group; or
11) i) R₁₁ is an electron acceptor group represented by Formula 1B, ii) R₁₂ and R₁₃ are each independently a group represented by one selected from Formulae 6-1 to 6-7, and iii) R₁₄ to R₂₀ and R₅₁ to R₅₃ are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group: 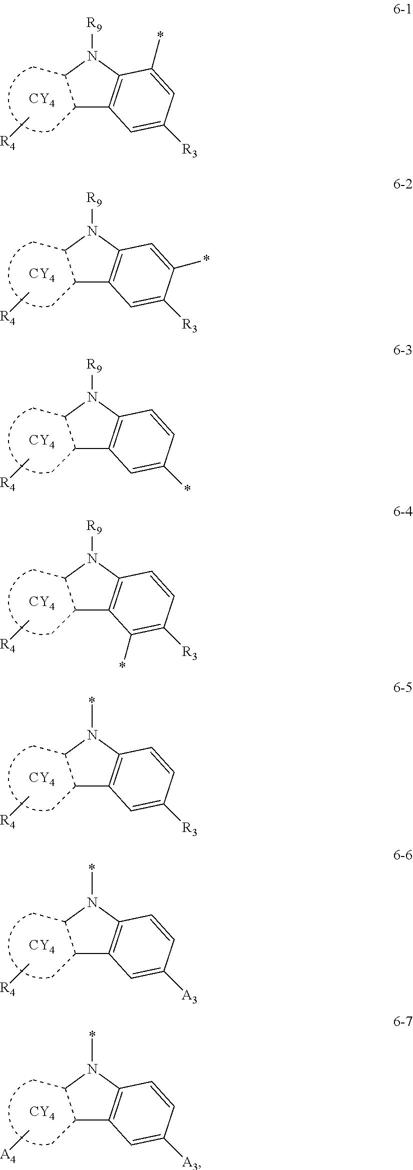
wherein, in Formulae 6-1 to 6-7,
CY₄ is a benzene group, a fluorene group, a dimehylfluorene group, a diphenylfluorene group, a carbazole group, a phehylcarbazole group, a biphenylcarbazole group, a dibenzofuran group, or a dibenzothiophene group,
R₃, R₄, and R₉ are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group,
A₃ and A₄ are each independently selected from acceptor groups represented by Formula 1B, and
“*” indicates a binding site to a neighboring atom.

14. The organic light-emitting device of claim 1, wherein
the fluorescent compound comprised in the emission layer is a fluorescent emitter, and
a proportion of fluorescent emission components of the fluorescent compound with respect to total emission components emitted from the emission layer is about 80% or more.

15. The organic light-emitting device of claim 14, wherein
the emission layer consists of the fluorescent compound; or
the emission layer comprises a fluorescent compound and further comprises a host.

16. The organic light-emitting device of claim 1, wherein
the emission layer comprises a host and a fluorescent dopant,
the host comprises the fluorescent compound, and
a proportion of fluorescent emission components of the fluorescent dopant with respect to total emission components emitted from the emission layer is about 80% or more.

17. The organic light-emitting device of claim 1, wherein
the emission layer comprises a host, an auxiliary dopant, and a fluorescent dopant,
the auxiliary dopant comprises the fluorescent compound, and
the emission layer satisfies Mathematical Expressions 1 and 2:
E_{T1(HOST)}−E_{T1(AD)}>0.05 electron volts Mathematical Expression 1
E_{S1(FD)}−E_{S1(AD)}<0 electron volts, Mathematical Expression 2
wherein, in Mathematical Expression 1, E_{T1(HOST)} is triplet energy (expressed in electron volts) of the host, and E_{T1(AD)} is triplet energy (expressed in electron volts) of the auxiliary dopant,
in Mathematical Expression 2, E_{S1(FD)} is singlet energy (expressed in electron volts) of the fluorescent dopant, and E_{S1(AD)} is singlet energy (expressed in electron volts) of the auxiliary dopant, and
E_{T1(HOST)}, E_{T1(AD)}, E_{S1(FD)}, and E_{S1(AD)} are each independently evaluated by using a Density Functional Theory (DFT) method of a Gaussian program that is structurally optimized at a level of B3LYP/6-31 G(d,p).

18. A compound represented by one of Formulae 9-1 to 9-9: 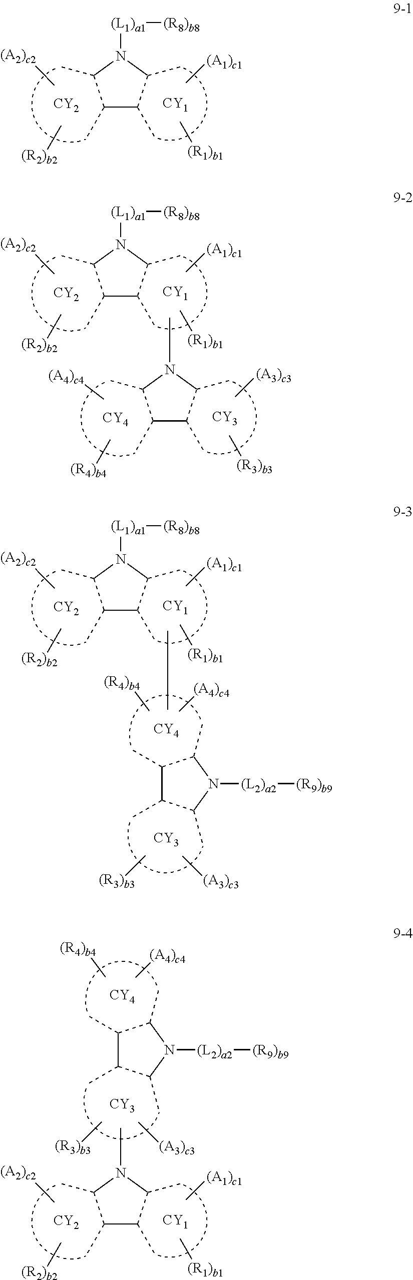 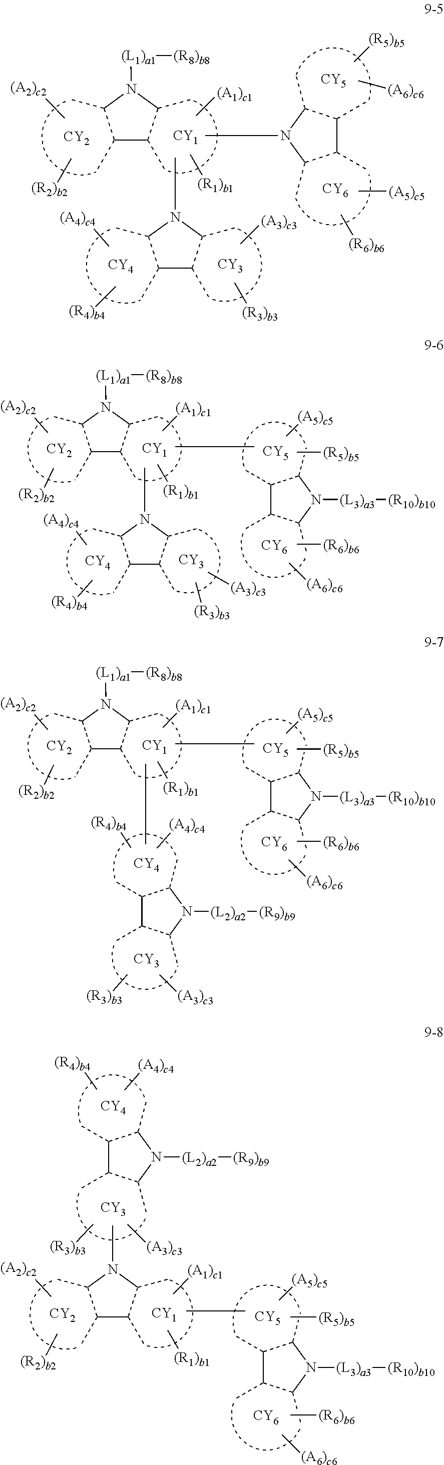 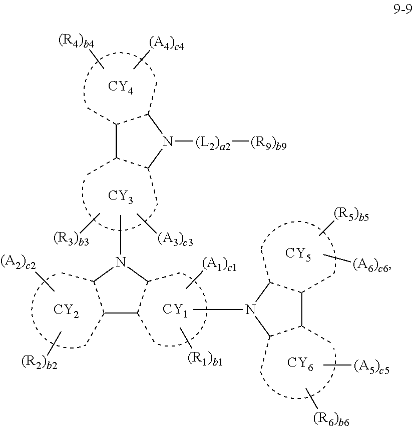
wherein, in Formulae 9-1 to 9-9,
CY₁ to CY₆ are each independently selected from a benzene group, a naphthalene group, a fluorene group, a carbazole group, a dibenzofuran group, and a dibenzothiophene group,
L₁ to L₃ are each independently selected from:
a single bond, a benzene group, a naphthalene group, a fluorene group, a carbazole group, a dibenzofuran group, and a dibenzothiophene group; and
a benzene group, a naphthalene group, a fluorene group, a carbazole group, a dibenzofuran group, and a dibenzothiophene group, each substituted with at least one selected from deuterium, a C₁-C₂₀ alkyl group, a C₁-C₂₀ alkoxy group, a phenyl group, a biphenyl group, a terphenyl group, a naphthyl group, a fluorenyl group, a dimethylfluorenyl group, a diphenylfluorenyl group, a carbazolyl group, a phenylcarbazolyl group, a biphenylcarbazolyl group, a dibenzofuranyl group, and a dibenzothiophenyl group,
a1 to a3 are each independently an integer from 1 to 3,
R₁ to R₆ and R₈ to R₁₀ are each independently selected from hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, a di(phenyl)phenyl group, a carbazolyl group, a (C₁-C₁₀ alkyl)carbazolyl group, a di(C₁-C₁₀ alkyl)carbazolyl group, a (phenyl)carbazolyl group, a di(phenyl)carbazolyl group, a fluorenyl group, a (C₁-C₁₀ alkyl)fluorenyl group, a di(C₁-C₁₀ alkyl)fluorenyl group, a (phenyl)fluorenyl group, a di(phenyl)fluorenyl group, a dibenzofuranyl group, a (C₁-C₁₀ alkyl)dibenzofuranyl group, a di(C₁-C₁₀ alkyl)dibenzofuranyl group, a (phenyl)dibenzofuranyl group, a di(phenyl)dibenzofuranyl group, a dibenzothiophenyl group, a (C₁-C₁₀ alkyl)dibenzothiophenyl group, a di(C₁-C₁₀ alkyl)dibenzothiophenyl group, a (phenyl)dibenzothiophenyl group, and a di(phenyl)dibenzothiophenyl group, provided that R₈ to R₁₀ are not hydrogen,
b1 to b6 are each independently 0, 1, or 2,
b8 to b10 are each independently 1 or 2,
A₁ to A₆ are each independently selected from electron acceptor groups represented by Formula 1B,
c1 to c6 are each independently 0, 1, 2, or 3, provided that the sum of c1 and c2 in Formula 9-1 is one or more, the sum of c1 to c4 in Formulae 9-2 to 9-4 is one or more, and the sum of c1 to c6 in Formulae 9-5 to 9-9 is one or more, and
a bond between A₁ and CY₁, a bond between A₂ and CY₂, a bond between A₃ and CY₃, a bond between A₄ and CY₄, a bond between A₅ and CY₅, and a bond between A₆ and CY₆ are each a carbon-carbon single bond:
*-(L₁₁)ₐ₁₁-(E₁₁)_{b11}, Formula 1B
wherein, in Formula 1B,
L₁₁ is selected from:
a single bond, a benzene group, a naphthalene group, a fluorene group, a pyridine group, a pyrazine group, a pyrimidine group, a pyridazine group, a quinoline group, an isoquinoline group, a benzoquinoline group, a quinoxaline group, a quinazoline group, and a triazine group; and
a benzene group, a naphthalene group, a fluorene group, a pyridine group, a pyrazine group, a pyrimidine group, a pyridazine group, a quinoline group, an isoquinoline group, a benzoquinoline group, a quinoxaline group, a quinazoline group, and a triazine group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a (C₁-C₂₀ alkyl)phenyl group, a di(C₁-C₂₀ alkyl)phenyl group, a (C₆-C₂₀ aryl)phenyl group, a di(C₆-C₂₀ aryl)phenyl group, a (C₃-C₂₀ heteroaryl)phenyl group, a di(C₃-C₂₀ heteroaryl)phenyl group, a pyridinyl group, a (C₁-C₂₀ alkyl)pyridinyl group, a di(C₁-C₂₀ alkyl)pyridinyl group, a (C₆-C₂₀ aryl)pyridinyl group, a di(C₆-C₂₀ aryl)pyridinyl group, a (C₃-C₂₀ heteroaryl)pyridinyl group, a di(C₃-C₂₀ heteroaryl)pyridinyl group, a pyrimidinyl group, a (C₁-C₂₀ alkyl)pyrimidinyl group, a di(C₁-C₂₀ alkyl)pyrimidinyl group, a (C₆-C₂₀ aryl)pyrimidinyl group, a di(C₆-C₂₀ aryl)pyrimidinyl group, a (C₃-C₂₀ heteroaryl)pyrimidinyl group, a di(C₃-C₂₀ heteroaryl)pyrimidinyl group, a triazinyl group, a (C₁-C₂₀ alkyl)triazinyl group, a di(C₁-C₂₀ alkyl)triazinyl group, a (C₆-C₂₀ aryl)triazinyl group, a di(C₆-C₂₀ aryl)triazinyl group, a (C₃-C₂₀ heteroaryl)triazinyl group, and a di(C₃-C₂₀ heteroaryl)triazinyl group,
a11 is 1 or 2,
E₁₁ is selected from:
—F, —CFH₂, —CF₂H, —CF₃, and —CN;
a C₁-C₂₀ alkyl group substituted with at least one selected from —F, —CFH₂, —CF₂H, —CF₃, and —CN; and
groups represented by Formulae 3-1 to 3-14,
b11 is an integer from 1 to 5,
a bond between L₁₁ and E₁₁ in Formula 1B is a carbon-carbon single bond or a carbon-fluorine single bond, and
“*” in Formula 1B indicates a binding site to a neighboring carbon: 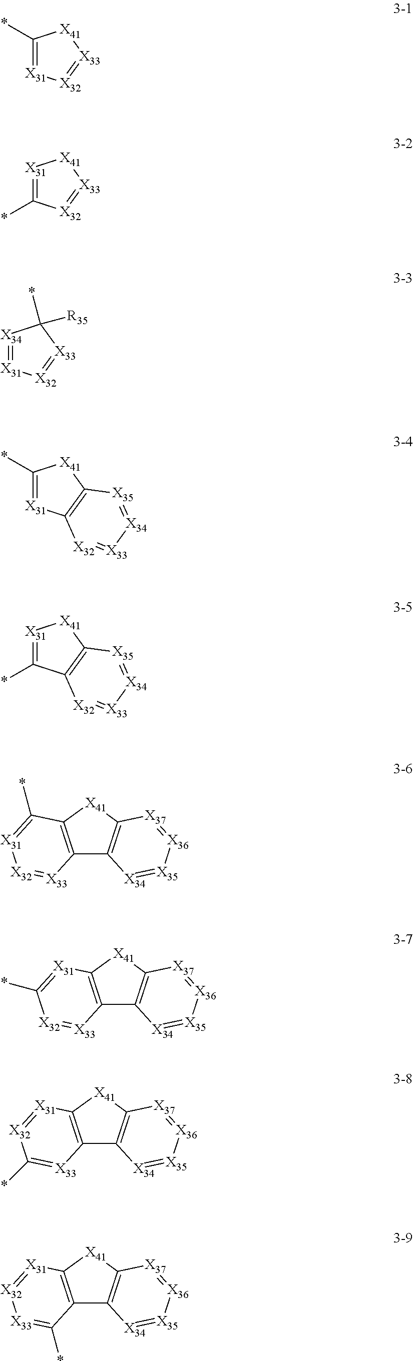 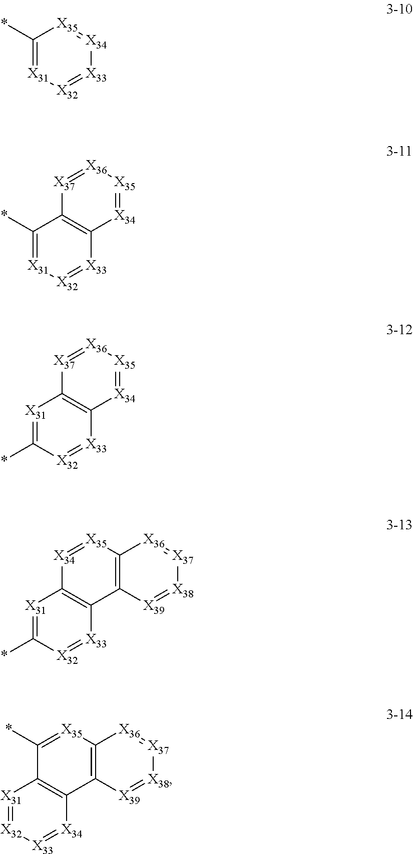
wherein, in Formulae 3-1 to 3-14, X₃₁ is N or C(R₃₁), X₃₂ is N or C(R₃₂), X₃₃ is N or C(R₃₃), X₃₄ is N or C(R₃₄), X₃₅ is N or C(R₃₅), X₃₆ is N or C(R₃₆), X₃₇ is N or C(R₃₇), X₃₈ is N or C(R₃₈), and X₃₉ is N or C(R₃₉),
X₄₁ in Formulae 3-1, 3-2, and 3-4 to 3-9 is N(R₄₁), C(R₄₂)(R₄₃), O, or S,
at least one of X₃₁ to X₃₃ in Formulae 3-1 and 3-2 is N, at least one of X₃₁ to X₃₄ in Formula 3-3 is N, at least one of X₃₁ to X₃₅ in Formulae 3-4, 3-5, and 3-10 is N, at least one of X₃₁ to X₃₇ in Formulae 3-6 to 3-9, 3-11, and 3-12 is N, and at least one of X₃₁ to X₃₉ in Formulae 3-13 and 3-14 is N,
R₃₁ to R₃₉ and R₄₁ to R₄₃ are each independently selected from hydrogen, deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a (C₁-C₂₀ alkyl)phenyl group, a di(C₁-C₂₀ alkyl)phenyl group, a (C₆-C₂₀ aryl)phenyl group, a di(C₆-C₂₀ aryl)phenyl group, a (C₃-C₂₀ heteroaryl)phenyl group, a di(C₃-C₂₀ heteroaryl)phenyl group, a pyridinyl group, a (C₁-C₂₀ alkyl)pyridinyl group, a di(C₁-C₂₀ alkyl)pyridinyl group, a (C₆-C₂₀ aryl)pyridinyl group, a di(C₆-C₂₀ aryl)pyridinyl group, a (C₃-C₂₀ heteroaryl)pyridinyl group, a di(C₃-C₂₀ heteroaryl)pyridinyl group, a pyrimidinyl group, a (C₁-C₂₀ alkyl)pyrimidinyl group, a di(C₁-C₂₀ alkyl)pyrimidinyl group, a (C₆-C₂₀ aryl)pyrimidinyl group, a di(C₆-C₂₀ aryl)pyrimidinyl group, a (C₃-C₂₀ heteroaryl)pyrimidinyl group, a di(C₃-C₂₀ heteroaryl)pyrimidinyl group, a triazinyl group, a (C₁-C₂₀ alkyl)triazinyl group, a di(C₁-C₂₀ alkyl)triazinyl group, a (C₆-C₂₀ aryl)triazinyl group, a di(C₆-C₂₀ aryl)triazinyl group, a (C₃-C₂₀ heteroaryl)triazinyl group, and a di(C₃-C₂₀ heteroaryl)triazinyl group, and
“*” indicates a binding site to a neighboring atom.

19. The compound of claim 18, wherein
the fluorescent compound is represented by one selected from Formulae 1-1 to 1-7: 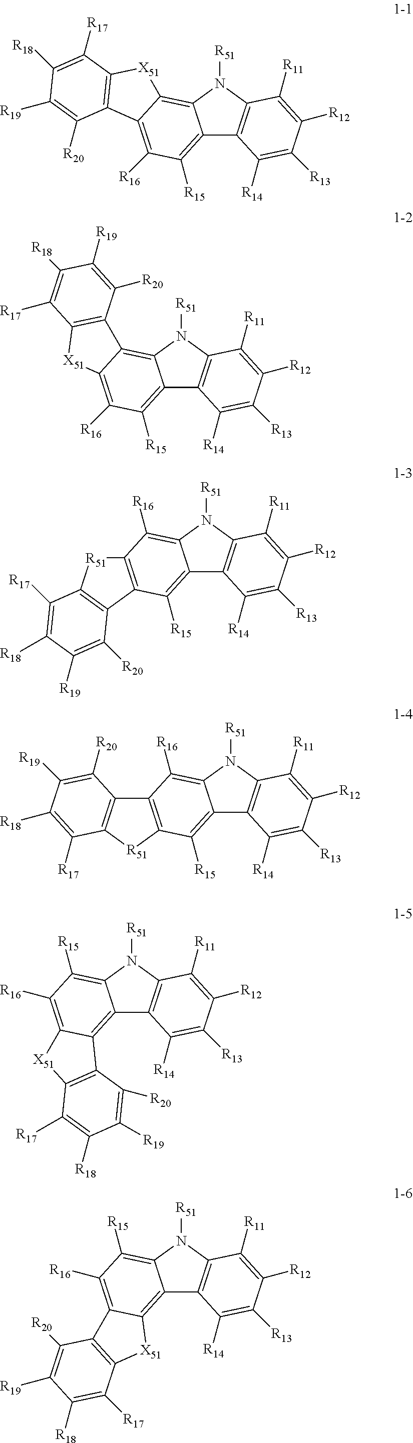 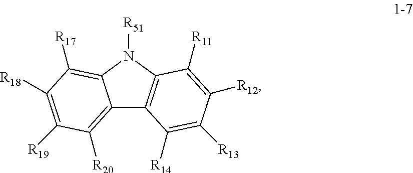
wherein, in Formulae 1-1 to 1-7,
X₅₁ is C(R₅₂)(R₅₃), N(R₅₂), O or S, and
1) i) R₁₁ is an electron acceptor group represented by Formula 1B, and ii) one selected from R₁₂ to R₂₀ and R₅₁ to R₅₃ is a group represented by one of Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
2) i) R₁₂ is an electron acceptor group represented by Formula 1B, and ii) one selected from R₁₁, R₁₃ to R₂₀, and R₅₁ to R₅₃ is a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
3) i) R₁₃ is an electron acceptor group represented by Formula 1B, and ii) one selected from R₁₁, R₁₂, R₁₄ to R₂₀, and R₅₁ to R₅₃ is a group represented by Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
4) i) R₁₄ is an electron acceptor group represented by Formula 1B, and ii) one selected from R₁₁ to R₁₃, R₁₅ to R₂₀, and R₅₁ to R₅₃ is a group a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
5) i) R₁₅ is an electron acceptor group represented by Formula 1B, and ii) one selected from R₁₁ to R₁₄, R₁₆ to R₂₀, and R₅₁ to R₅₃ is a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
6) i) R₁₆ is an electron acceptor group represented by Formula 1B, and ii) one selected from R₁₁ to R₁₅, R₁₇ to R₂₀, and R₅₁ to R₅₃ is a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
7) i) R₁₂ is an electron acceptor group represented by Formula 1B, ii) R₁₁ and R₁₃ are each independently a group represented by one selected from Formulae 6-1 to 6-7, and iii) R₁₄ to R₂₀ and R₅₁ to R₅₃ are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
8) i) R₁₃ is an electron acceptor group represented by Formula 1B, ii) R₁₂ and R₁₄ are each independently a group represented by one selected from Formulae 6-1 to 6-7, and iii) R₁₁, R₁₅ to R₂₀, and R₅₁ to R₅₃ are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
9) i) R₁₂ and R₁₃ are each independently an electron acceptor group represented by Formula 1B, and ii) one selected from R₁₁, R₁₄ to R₂₀, and R₅₁ to R₅₃ is a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group;
10) i) R₁₁ and R₁₂ are each independently an electron acceptor group represented by Formula 1B, and ii) one selected from R₁₃ to R₂₀ and R₅₁ to R₅₃ is a group represented by one selected from Formulae 6-1 to 6-7, and the rest are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group; or
11) i) R₁₁ is an electron acceptor group represented by Formula 1B, ii) R₁₂ and R₁₃ are each independently a group represented by one selected from Formulae 6-1 to 6-7, and iii) R₁₄ to R₂₀ and R₅₁ to R₅₃ are each independently hydrogen, deuterium, a C₁-C₁₀ alkyl group, a phenyl group, a (C₁-C₁₀ alkyl)phenyl group, a di(C₁-C₁₀ alkyl)phenyl group, a biphenyl group, or a di(phenyl)phenyl group.

20. The compound of claim 18, wherein
the compound is one of Compounds 1 to 936: 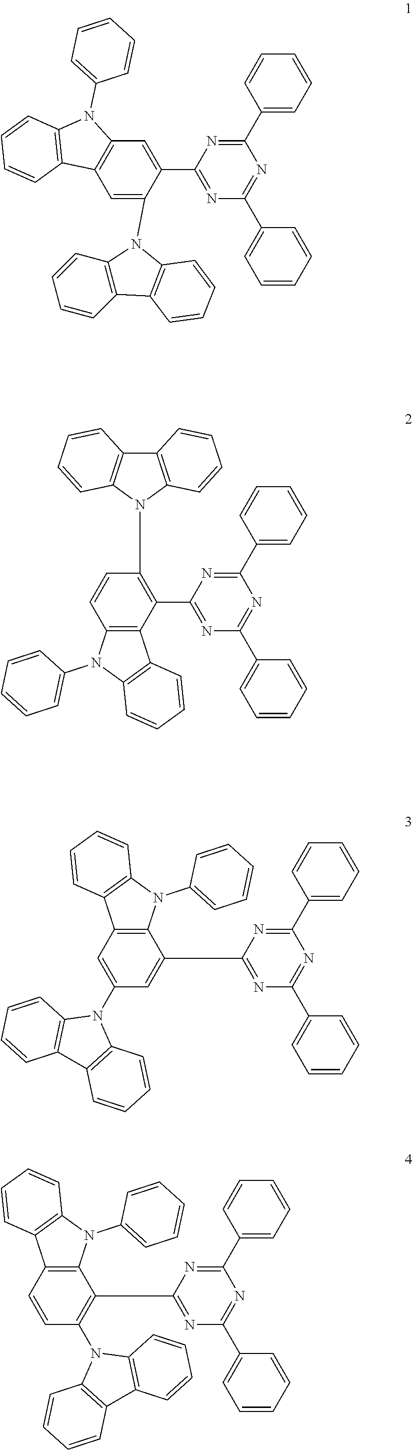 [SMILES: c1ccc(-c2nc(-c3ccccc3)nc(-c3cc4c(cc3-n3c5ccccc5c5ccccc53)c3ccccc3n4-c3ccccc3)n2)cc1 ; c1ccc(-c2nc(-c3ccccc3)nc(-c3c(-n4c5ccccc5c5ccccc54)ccc4c3c3ccccc3n4-c3ccccc3)n2)cc1 ; c1ccc(-c2nc(-c3ccccc3)nc(-c3cc(-n4c5ccccc5c5ccccc54)cc4c5ccccc5n(-c5ccccc5)c34)n2)cc1 ; c1ccc(-c2nc(-c3ccccc3)nc(-c3c(-n4c5ccccc5c5ccccc54)ccc4c5ccccc5n(-c5ccccc5)c34)n2)cc1] 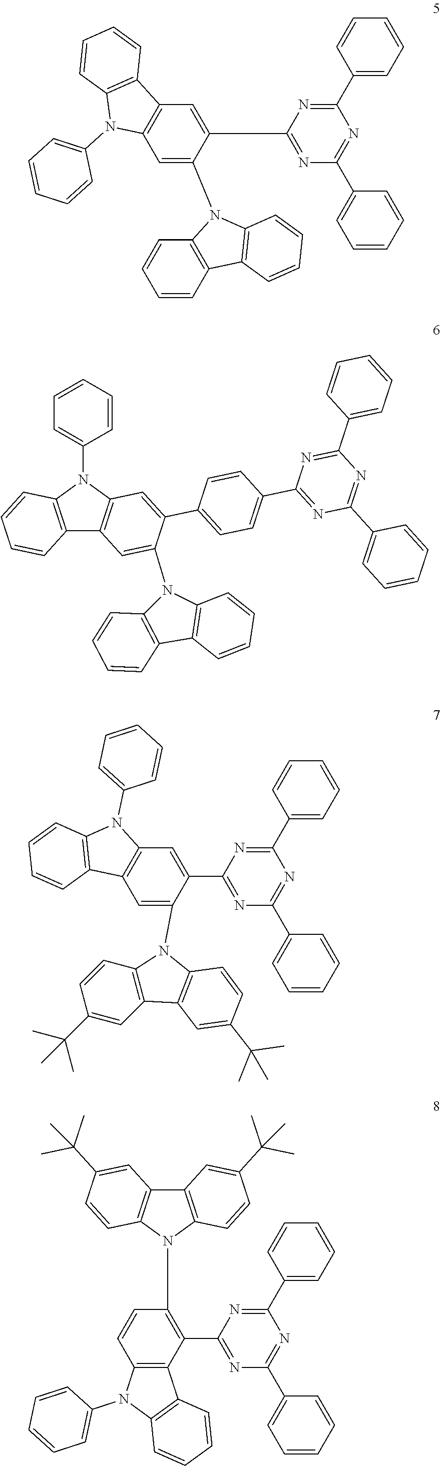 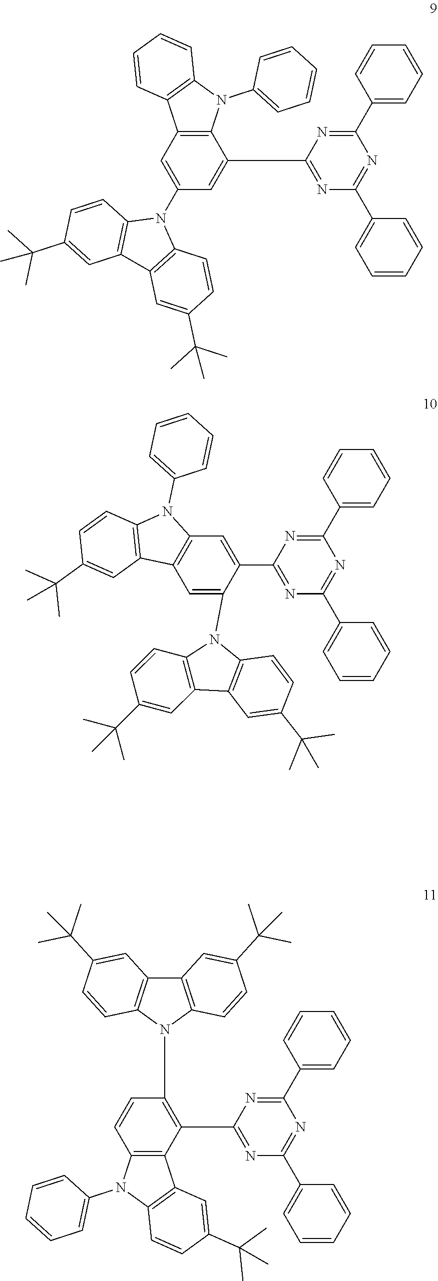 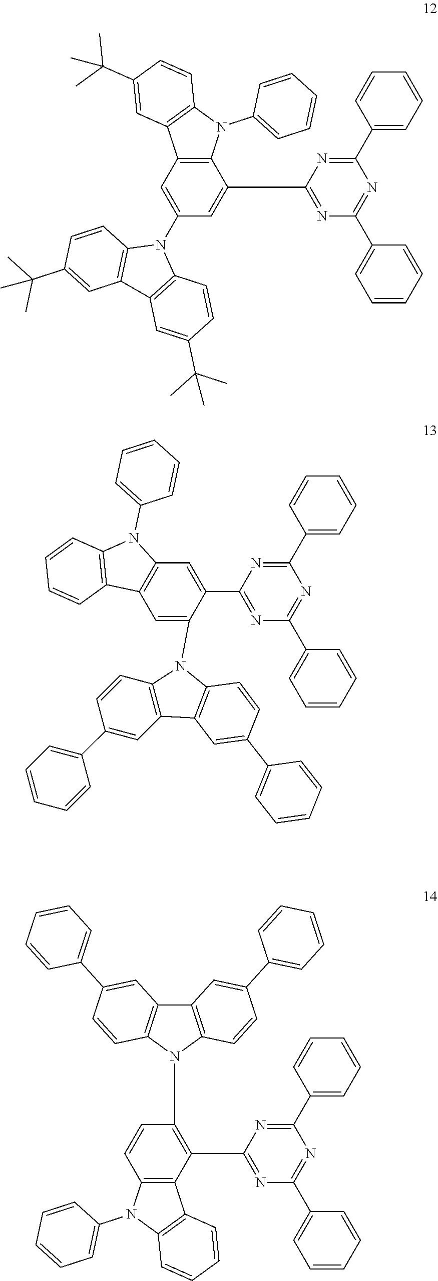 [SMILES: CC(C)(C)c1ccc2c(c1)c1cc(C(C)(C)C)ccc1n2-c1cc(-c2nc(-c3ccccc3)nc(-c3ccccc3)n2)c2c(c1)c1cc(C(C)(C)C)ccc1n2-c1ccccc1 ; c1ccc(-c2ccc3c(c2)c2cc(-c4ccccc4)ccc2n3-c2cc3c4ccccc4n(-c4ccccc4)c3cc2-c2nc(-c3ccccc3)nc(-c3ccccc3)n2)cc1 ; c1ccc(-c2ccc3c(c2)c2cc(-c4ccccc4)ccc2n3-c2ccc3c(c2-c2nc(-c4ccccc4)nc(-c4ccccc4)n2)c2ccccc2n3-c2ccccc2)cc1] 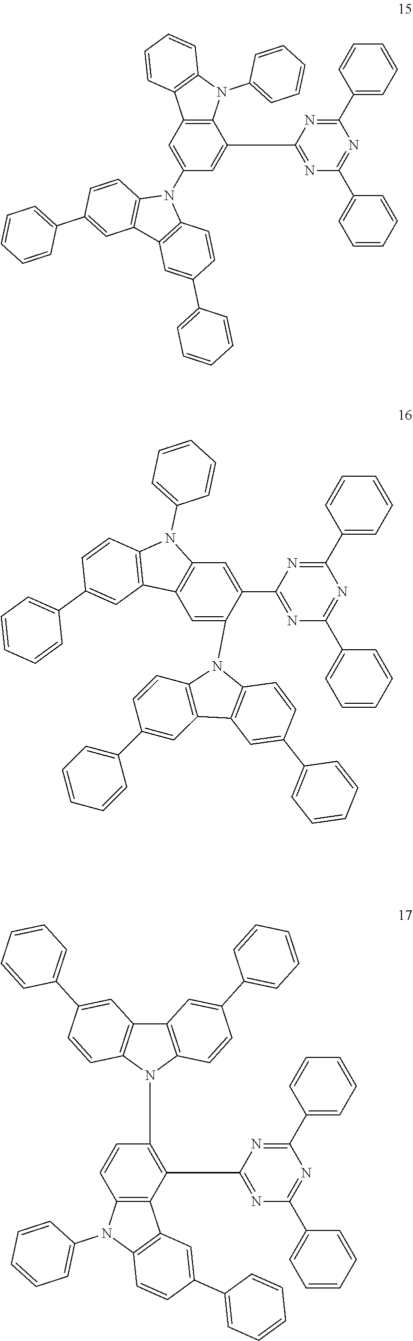 [SMILES: c1ccc(-c2ccc3c(c2)c2cc(-c4ccccc4)ccc2n3-c2cc(-c3nc(-c4ccccc4)nc(-c4ccccc4)n3)c3c(c2)c2ccccc2n3-c2ccccc2)cc1 ; c1ccc(-c2ccc3c(c2)c2cc(-n4c5ccc(-c6ccccc6)cc5c5cc(-c6ccccc6)ccc54)c(-c4nc(-c5ccccc5)nc(-c5ccccc5)n4)cc2n3-c2ccccc2)cc1 ; c1ccc(-c2ccc3c(c2)c2cc(-c4ccccc4)ccc2n3-c2ccc3c(c2-c2nc(-c4ccccc4)nc(-c4ccccc4)n2)c2cc(-c4ccccc4)ccc2n3-c2ccccc2)cc1] 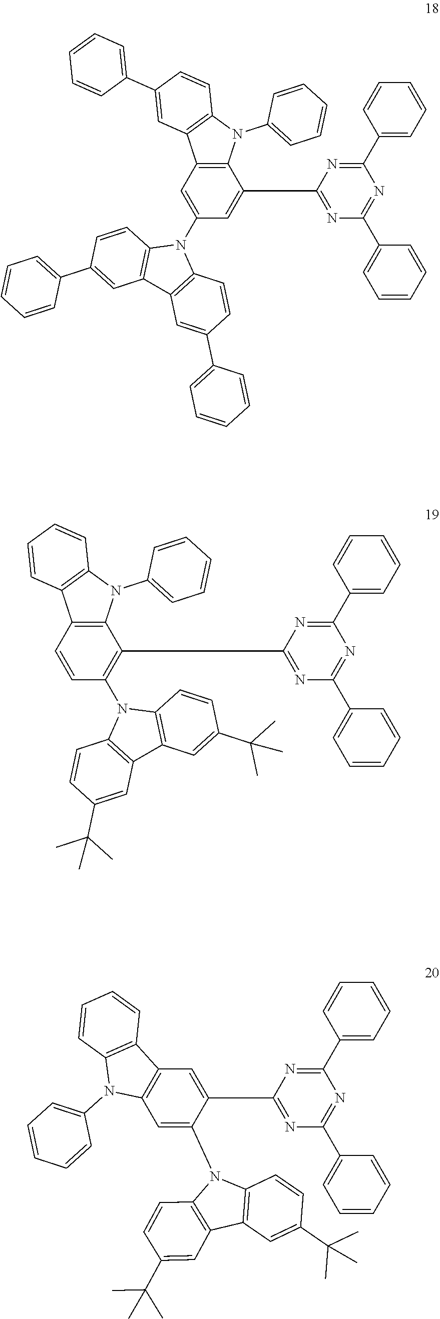 [SMILES: c1ccc(-c2ccc3c(c2)c2cc(-c4ccccc4)ccc2n3-c2cc(-c3nc(-c4ccccc4)nc(-c4ccccc4)n3)c3c(c2)c2cc(-c4ccccc4)ccc2n3-c2ccccc2)cc1 ; CC(C)(C)c1ccc2c(c1)c1cc(C(C)(C)C)ccc1n2-c1ccc2c3ccccc3n(-c3ccccc3)c2c1-c1nc(-c2ccccc2)nc(-c2ccccc2)n1 ; CC(C)(C)c1ccc2c(c1)c1cc(C(C)(C)C)ccc1n2-c1cc2c(cc1-c1nc(-c3ccccc3)nc(-c3ccccc3)n1)c1ccccc1n2-c1ccccc1] 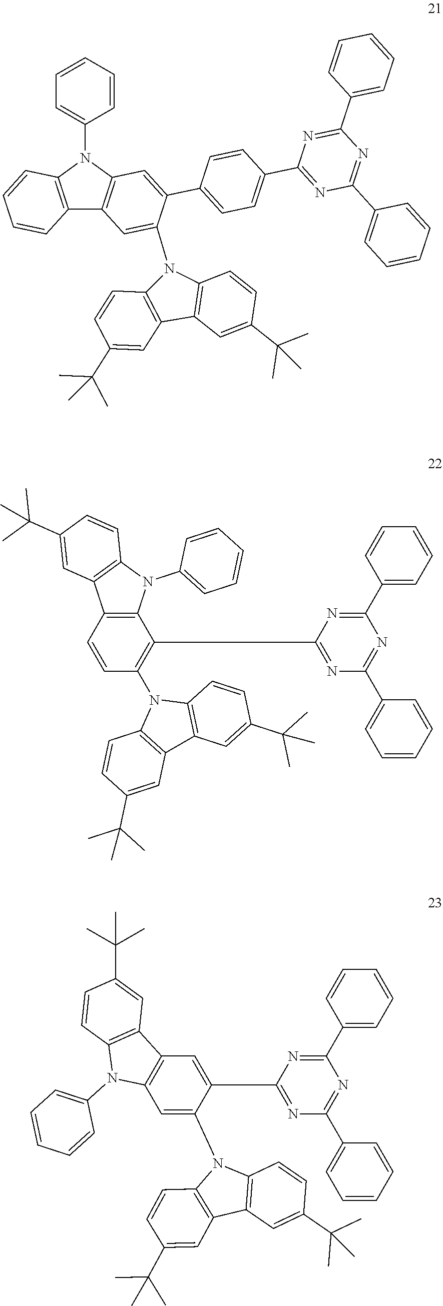 [SMILES: CC(C)(C)c1ccc2c(c1)c1cc(C(C)(C)C)ccc1n2-c1cc2c3ccccc3n(-c3ccccc3)c2cc1-c1ccc(-c2nc(-c3ccccc3)nc(-c3ccccc3)n2)cc1 ; CC(C)(C)c1ccc2c(c1)c1cc(C(C)(C)C)ccc1n2-c1ccc2c3cc(C(C)(C)C)ccc3n(-c3ccccc3)c2c1-c1nc(-c2ccccc2)nc(-c2ccccc2)n1 ; CC(C)(C)c1ccc2c(c1)c1cc(-c3nc(-c4ccccc4)nc(-c4ccccc4)n3)c(-n3c4ccc(C(C)(C)C)cc4c4cc(C(C)(C)C)ccc43)cc1n2-c1ccccc1] 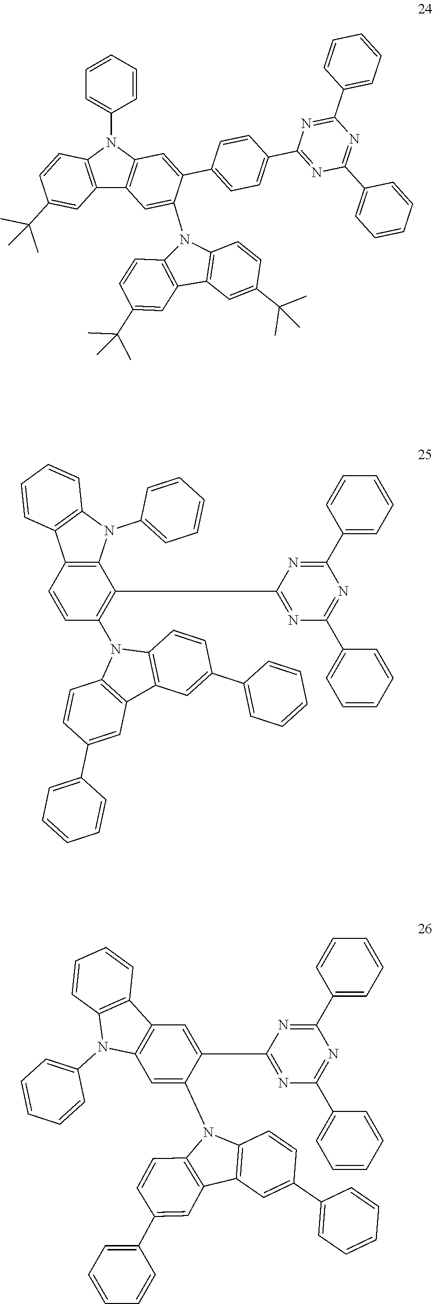 [SMILES: CC(C)(C)c1ccc2c(c1)c1cc(-n3c4ccc(C(C)(C)C)cc4c4cc(C(C)(C)C)ccc43)c(-c3ccc(-c4nc(-c5ccccc5)nc(-c5ccccc5)n4)cc3)cc1n2-c1ccccc1 ; c1ccc(-c2ccc3c(c2)c2cc(-c4ccccc4)ccc2n3-c2ccc3c4ccccc4n(-c4ccccc4)c3c2-c2nc(-c3ccccc3)nc(-c3ccccc3)n2)cc1 ; c1ccc(-c2ccc3c(c2)c2cc(-c4ccccc4)ccc2n3-c2cc3c(cc2-c2nc(-c4ccccc4)nc(-c4ccccc4)n2)c2ccccc2n3-c2ccccc2)cc1] 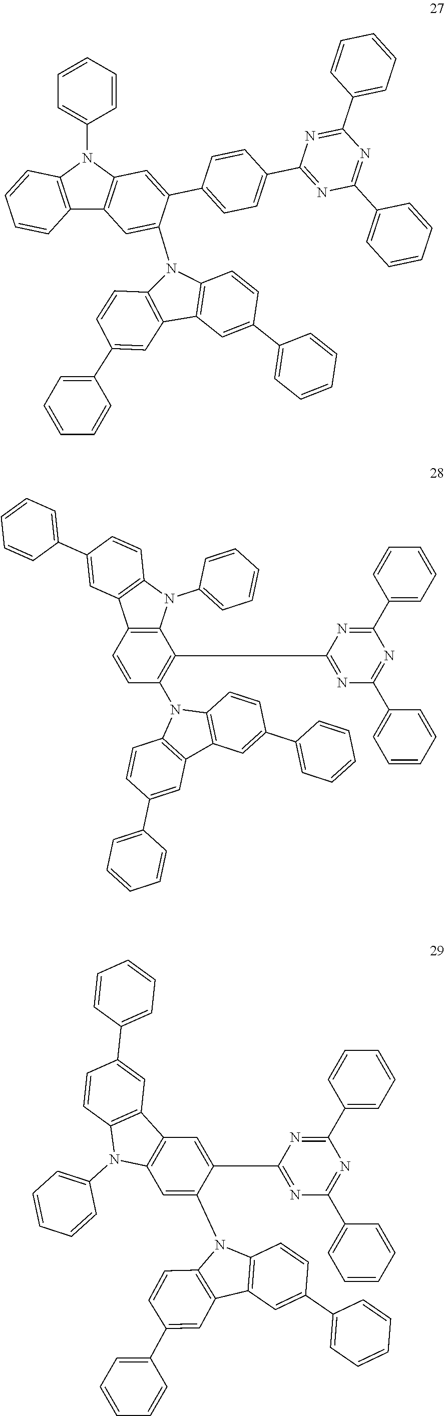 [SMILES: c1ccc(-c2ccc3c(c2)c2cc(-c4ccccc4)ccc2n3-c2cc3c4ccccc4n(-c4ccccc4)c3cc2-c2ccc(-c3nc(-c4ccccc4)nc(-c4ccccc4)n3)cc2)cc1 ; c1ccc(-c2ccc3c(c2)c2cc(-c4ccccc4)ccc2n3-c2ccc3c4cc(-c5ccccc5)ccc4n(-c4ccccc4)c3c2-c2nc(-c3ccccc3)nc(-c3ccccc3)n2)cc1 ; c1ccc(-c2ccc3c(c2)c2cc(-c4nc(-c5ccccc5)nc(-c5ccccc5)n4)c(-n4c5ccc(-c6ccccc6)cc5c5cc(-c6ccccc6)ccc54)cc2n3-c2ccccc2)cc1] 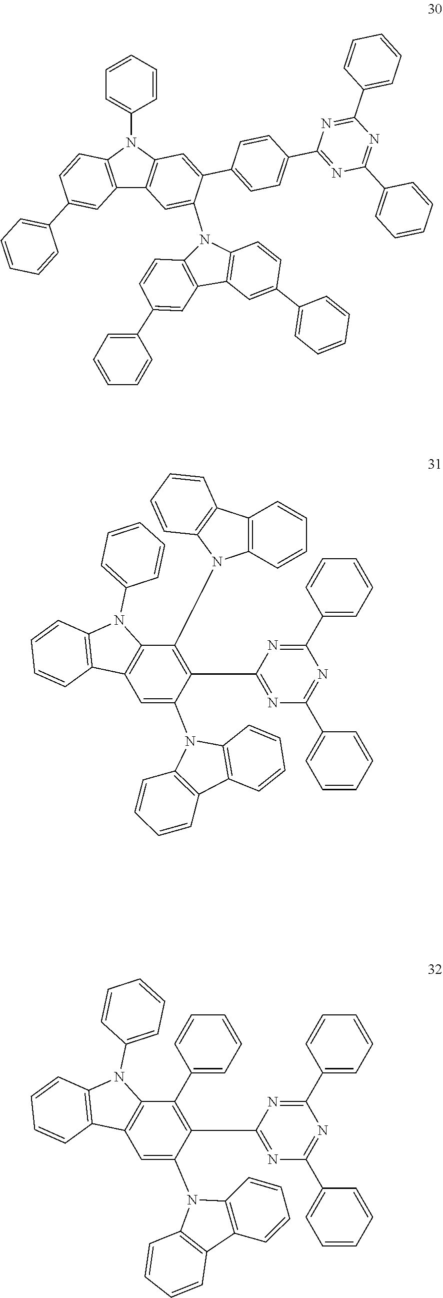 [SMILES: c1ccc(-c2ccc3c(c2)c2cc(-n4c5ccc(-c6ccccc6)cc5c5cc(-c6ccccc6)ccc54)c(-c4ccc(-c5nc(-c6ccccc6)nc(-c6ccccc6)n5)cc4)cc2n3-c2ccccc2)cc1 ; c1ccc(-c2nc(-c3ccccc3)nc(-c3c(-n4c5ccccc5c5ccccc54)cc4c5ccccc5n(-c5ccccc5)c4c3-n3c4ccccc4c4ccccc43)n2)cc1 ; c1ccc(-c2nc(-c3ccccc3)nc(-c3c(-n4c5ccccc5c5ccccc54)cc4c5ccccc5n(-c5ccccc5)c4c3-c3ccccc3)n2)cc1] 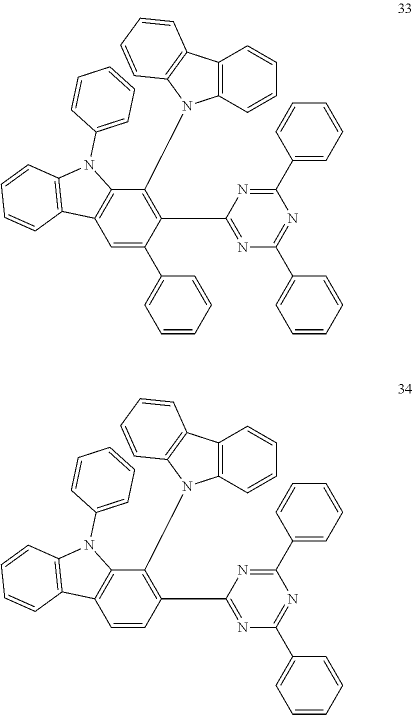 [SMILES: c1ccc(-c2nc(-c3ccccc3)nc(-c3c(-c4ccccc4)cc4c5ccccc5n(-c5ccccc5)c4c3-n3c4ccccc4c4ccccc43)n2)cc1 ; c1ccc(-c2nc(-c3ccccc3)nc(-c3ccc4c5ccccc5n(-c5ccccc5)c4c3-n3c4ccccc4c4ccccc43)n2)cc1] 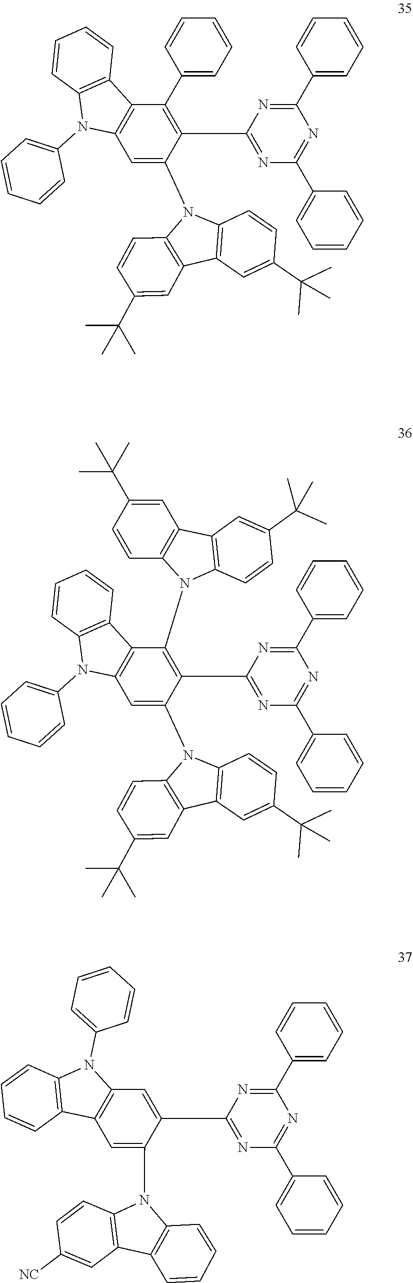 [SMILES: CC(C)(C)c1ccc2c(c1)c1cc(C(C)(C)C)ccc1n2-c1cc2c(c(-c3ccccc3)c1-c1nc(-c3ccccc3)nc(-c3ccccc3)n1)c1ccccc1n2-c1ccccc1 ; CC(C)(C)c1ccc2c(c1)c1cc(C(C)(C)C)ccc1n2-c1cc2c(c(-n3c4ccc(C(C)(C)C)cc4c4cc(C(C)(C)C)ccc43)c1-c1nc(-c3ccccc3)nc(-c3ccccc3)n1)c1ccccc1n2-c1ccccc1 ; N#Cc1ccc2c(c1)c1ccccc1n2-c1cc2c3ccccc3n(-c3ccccc3)c2cc1-c1nc(-c2ccccc2)nc(-c2ccccc2)n1] 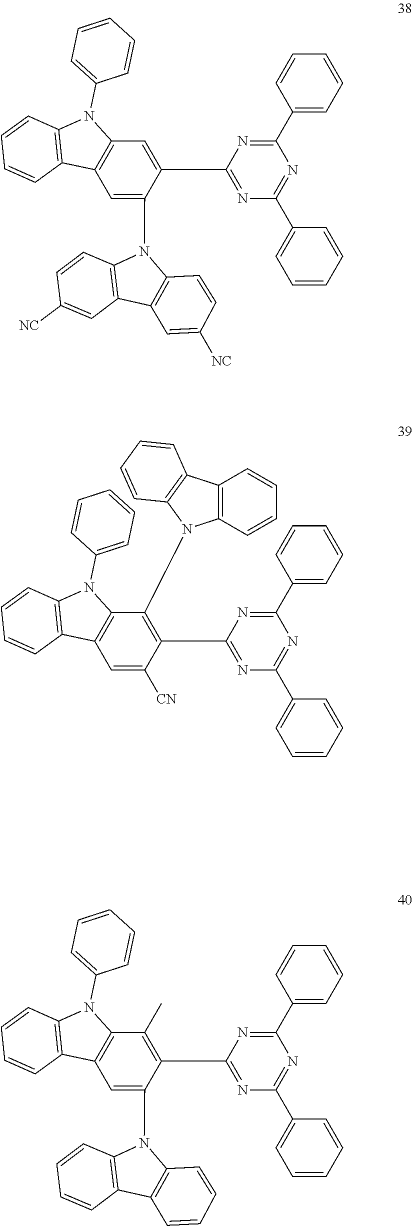 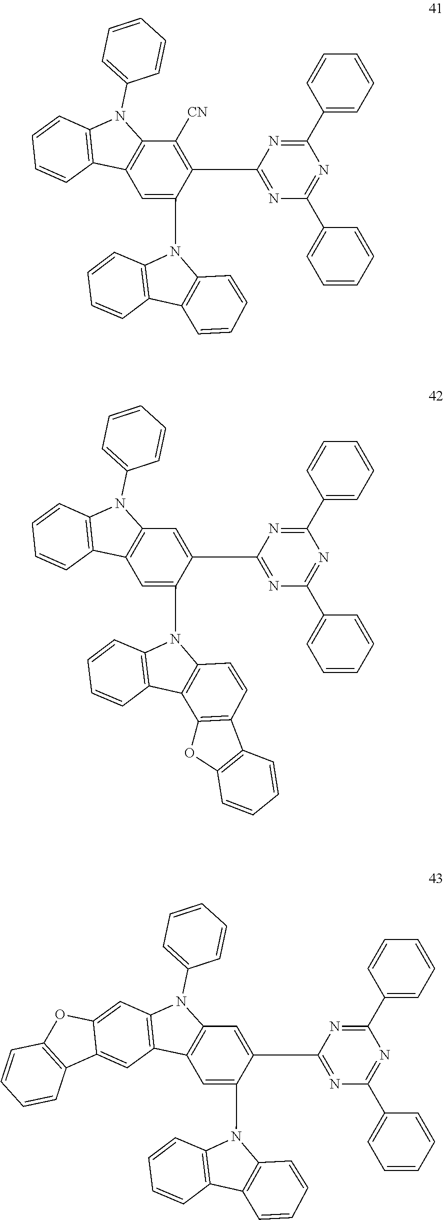 [SMILES: N#Cc1c(-c2nc(-c3ccccc3)nc(-c3ccccc3)n2)c(-n2c3ccccc3c3ccccc32)cc2c3ccccc3n(-c3ccccc3)c12 ; c1ccc(-c2nc(-c3ccccc3)nc(-c3cc4c(cc3-n3c5ccccc5c5c6oc7ccccc7c6ccc53)c3ccccc3n4-c3ccccc3)n2)cc1 ; c1ccc(-c2nc(-c3ccccc3)nc(-c3cc4c(cc3-n3c5ccccc5c5ccccc53)c3cc5c(cc3n4-c3ccccc3)oc3ccccc35)n2)cc1] 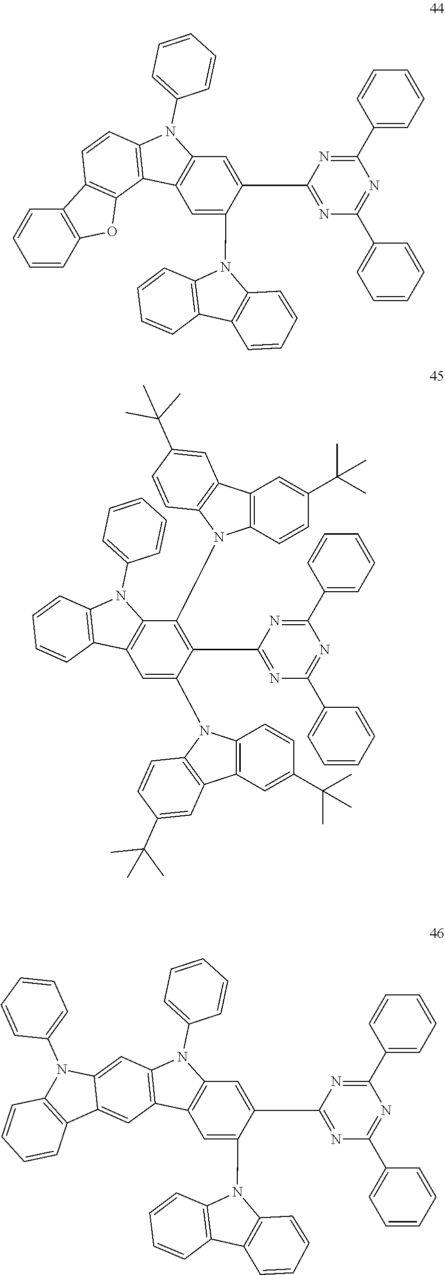 [SMILES: c1ccc(-c2nc(-c3ccccc3)nc(-c3cc4c(cc3-n3c5ccccc5c5ccccc53)c3c5oc6ccccc6c5ccc3n4-c3ccccc3)n2)cc1 ; CC(C)(C)c1ccc2c(c1)c1cc(C(C)(C)C)ccc1n2-c1cc2c3ccccc3n(-c3ccccc3)c2c(-n2c3ccc(C(C)(C)C)cc3c3cc(C(C)(C)C)ccc32)c1-c1nc(-c2ccccc2)nc(-c2ccccc2)n1 ; c1ccc(-c2nc(-c3ccccc3)nc(-c3cc4c(cc3-n3c5ccccc5c5ccccc53)c3cc5c6ccccc6n(-c6ccccc6)c5cc3n4-c3ccccc3)n2)cc1] 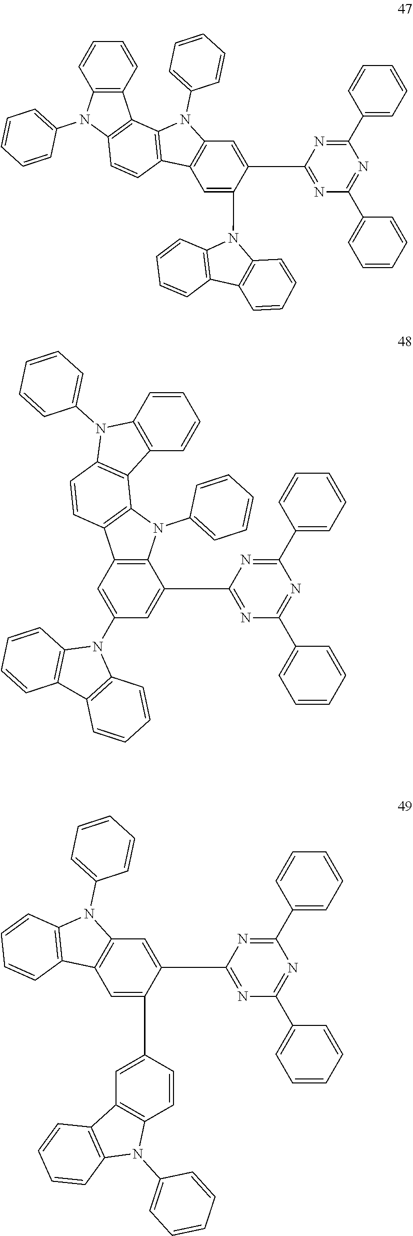 [SMILES: c1ccc(-c2nc(-c3ccccc3)nc(-c3cc4c(cc3-n3c5ccccc5c5ccccc53)c3ccc5c(c6ccccc6n5-c5ccccc5)c3n4-c3ccccc3)n2)cc1 ; c1ccc(-c2nc(-c3ccccc3)nc(-c3cc(-n4c5ccccc5c5ccccc54)cc4c5ccc6c(c7ccccc7n6-c6ccccc6)c5n(-c5ccccc5)c34)n2)cc1 ; c1ccc(-c2nc(-c3ccccc3)nc(-c3cc4c(cc3-c3ccc5c(c3)c3ccccc3n5-c3ccccc3)c3ccccc3n4-c3ccccc3)n2)cc1] 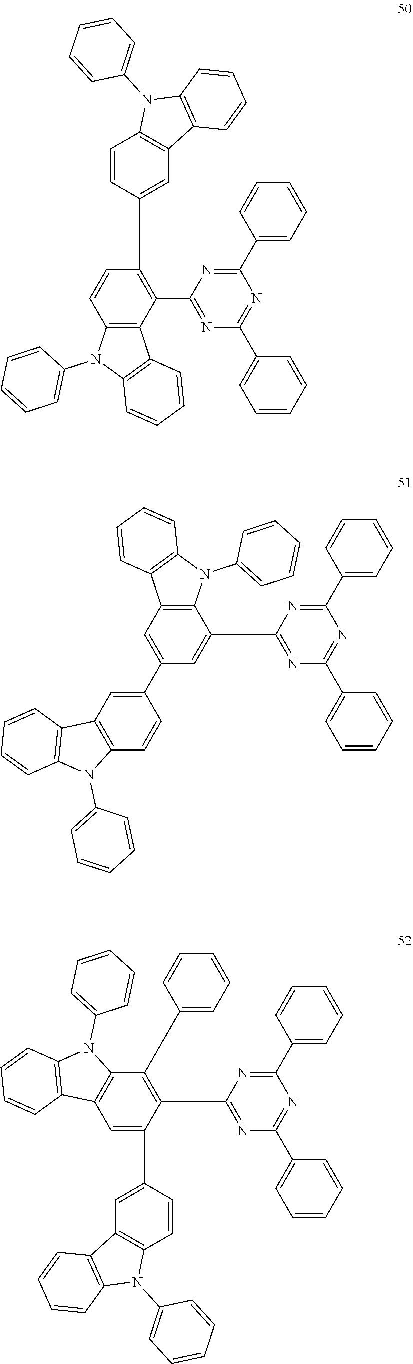 [SMILES: c1ccc(-c2nc(-c3ccccc3)nc(-c3c(-c4ccc5c(c4)c4ccccc4n5-c4ccccc4)ccc4c3c3ccccc3n4-c3ccccc3)n2)cc1 ; c1ccc(-c2nc(-c3ccccc3)nc(-c3cc(-c4ccc5c(c4)c4ccccc4n5-c4ccccc4)cc4c5ccccc5n(-c5ccccc5)c34)n2)cc1 ; c1ccc(-c2nc(-c3ccccc3)nc(-c3c(-c4ccc5c(c4)c4ccccc4n5-c4ccccc4)cc4c5ccccc5n(-c5ccccc5)c4c3-c3ccccc3)n2)cc1] 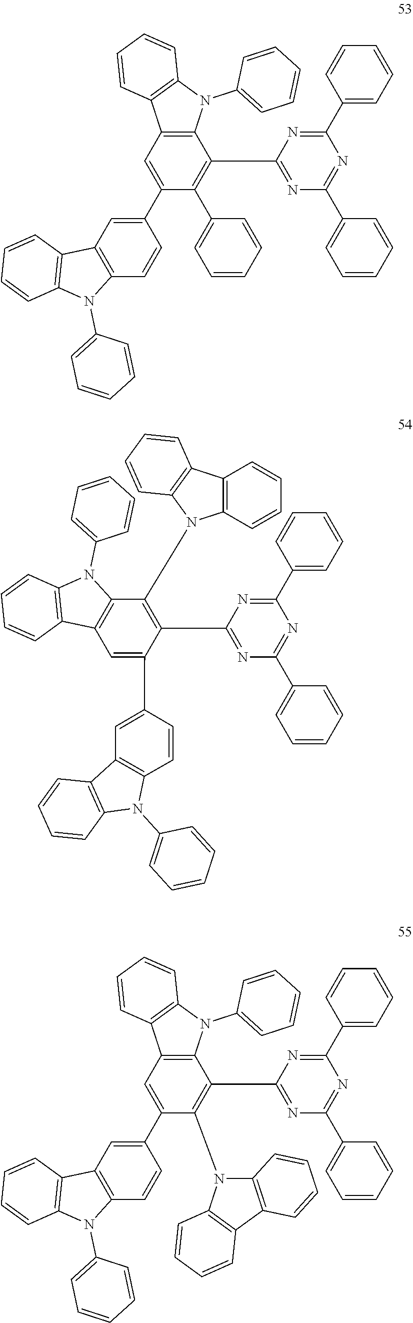 [SMILES: c1ccc(-c2nc(-c3ccccc3)nc(-c3c(-c4ccccc4)c(-c4ccc5c(c4)c4ccccc4n5-c4ccccc4)cc4c5ccccc5n(-c5ccccc5)c34)n2)cc1 ; c1ccc(-c2nc(-c3ccccc3)nc(-c3c(-c4ccc5c(c4)c4ccccc4n5-c4ccccc4)cc4c5ccccc5n(-c5ccccc5)c4c3-n3c4ccccc4c4ccccc43)n2)cc1 ; c1ccc(-c2nc(-c3ccccc3)nc(-c3c(-n4c5ccccc5c5ccccc54)c(-c4ccc5c(c4)c4ccccc4n5-c4ccccc4)cc4c5ccccc5n(-c5ccccc5)c34)n2)cc1] 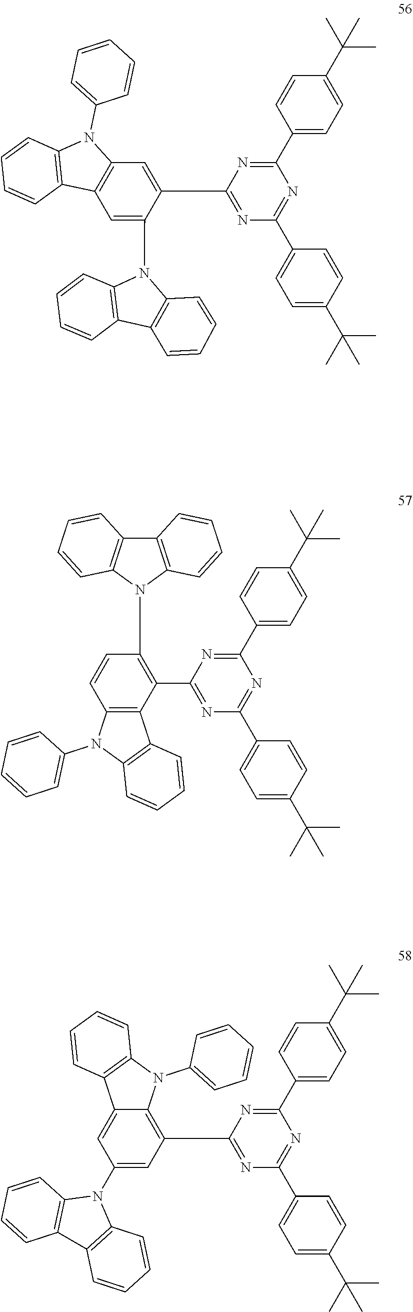 [SMILES: CC(C)(C)c1ccc(-c2nc(-c3ccc(C(C)(C)C)cc3)nc(-c3cc4c(cc3-n3c5ccccc5c5ccccc53)c3ccccc3n4-c3ccccc3)n2)cc1 ; CC(C)(C)c1ccc(-c2nc(-c3ccc(C(C)(C)C)cc3)nc(-c3c(-n4c5ccccc5c5ccccc54)ccc4c3c3ccccc3n4-c3ccccc3)n2)cc1 ; CC(C)(C)c1ccc(-c2nc(-c3ccc(C(C)(C)C)cc3)nc(-c3cc(-n4c5ccccc5c5ccccc54)cc4c5ccccc5n(-c5ccccc5)c34)n2)cc1] 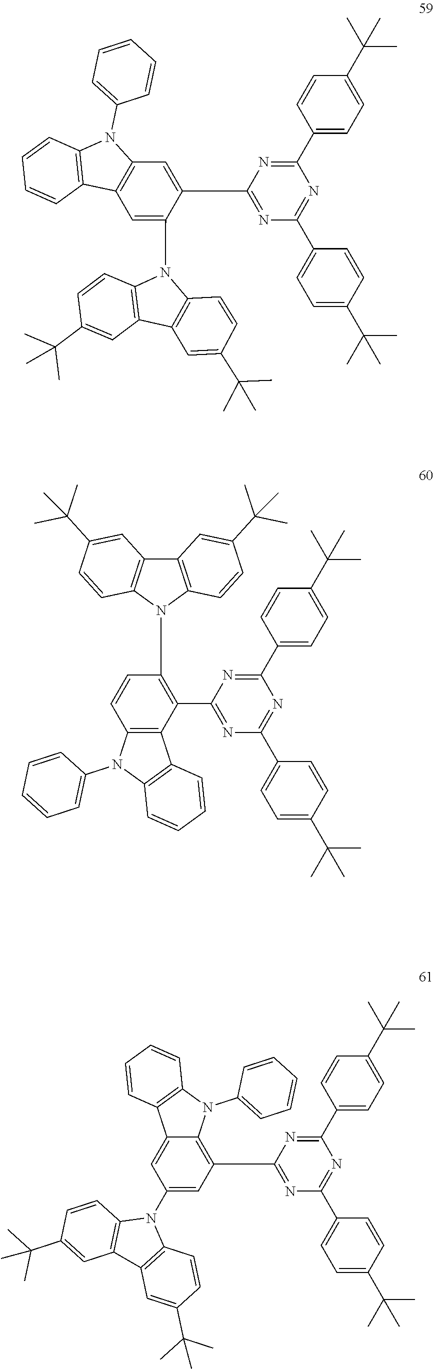 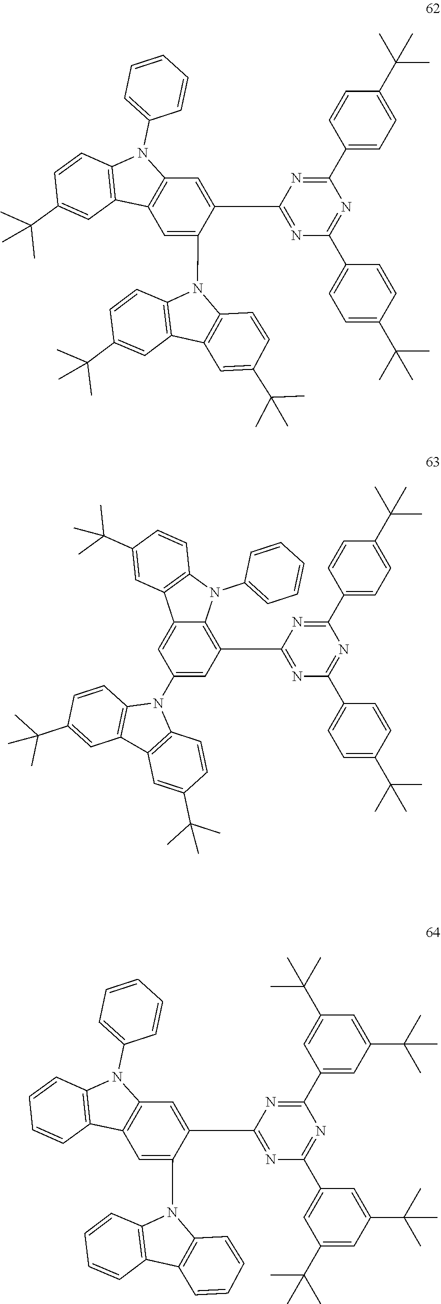 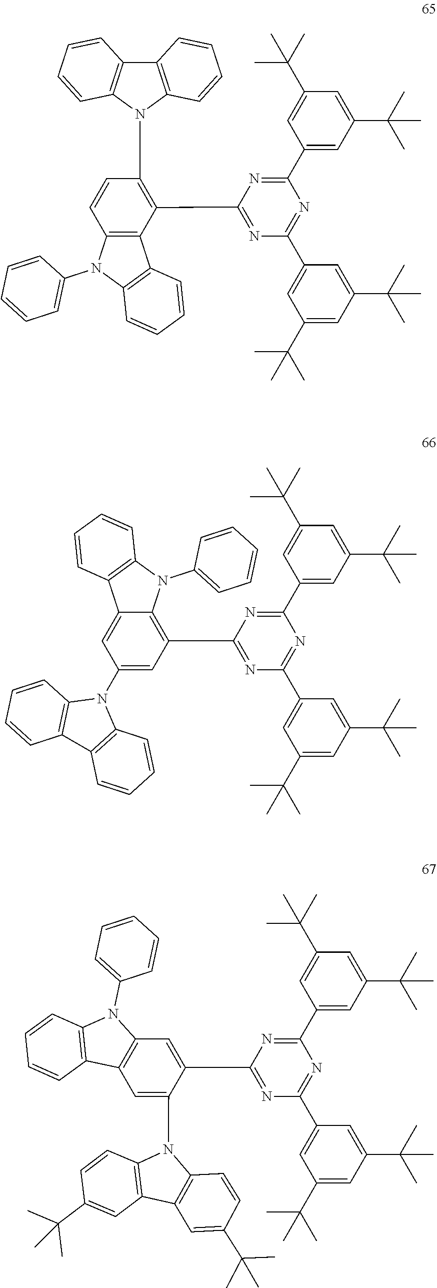 [SMILES: CC(C)(C)c1cc(-c2nc(-c3cc(C(C)(C)C)cc(C(C)(C)C)c3)nc(-c3c(-n4c5ccccc5c5ccccc54)ccc4c3c3ccccc3n4-c3ccccc3)n2)cc(C(C)(C)C)c1 ; CC(C)(C)c1cc(-c2nc(-c3cc(C(C)(C)C)cc(C(C)(C)C)c3)nc(-c3cc(-n4c5ccccc5c5ccccc54)cc4c5ccccc5n(-c5ccccc5)c34)n2)cc(C(C)(C)C)c1 ; CC(C)(C)c1cc(-c2nc(-c3cc(C(C)(C)C)cc(C(C)(C)C)c3)nc(-c3cc4c(cc3-n3c5ccc(C(C)(C)C)cc5c5cc(C(C)(C)C)ccc53)c3ccccc3n4-c3ccccc3)n2)cc(C(C)(C)C)c1] 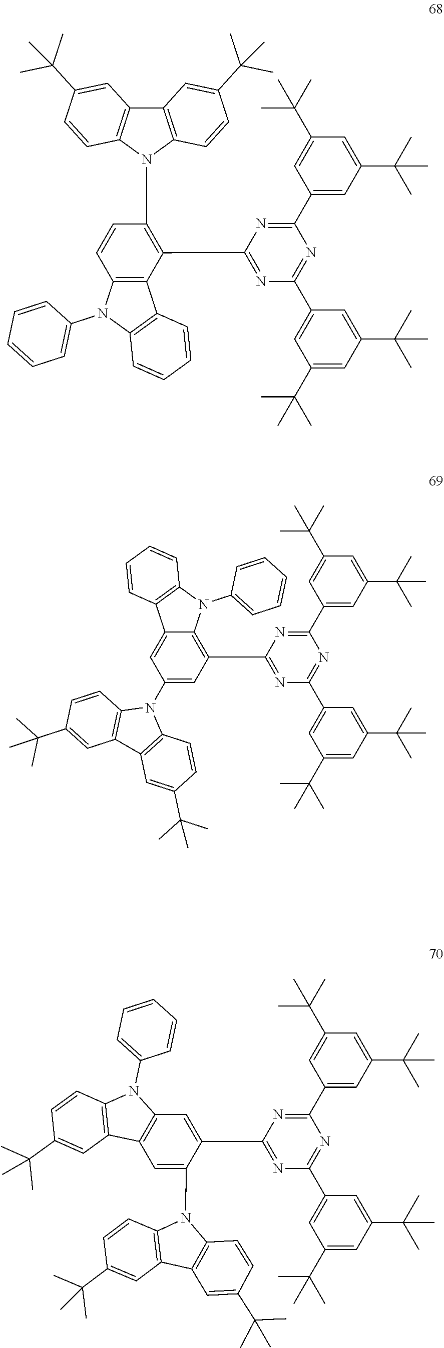 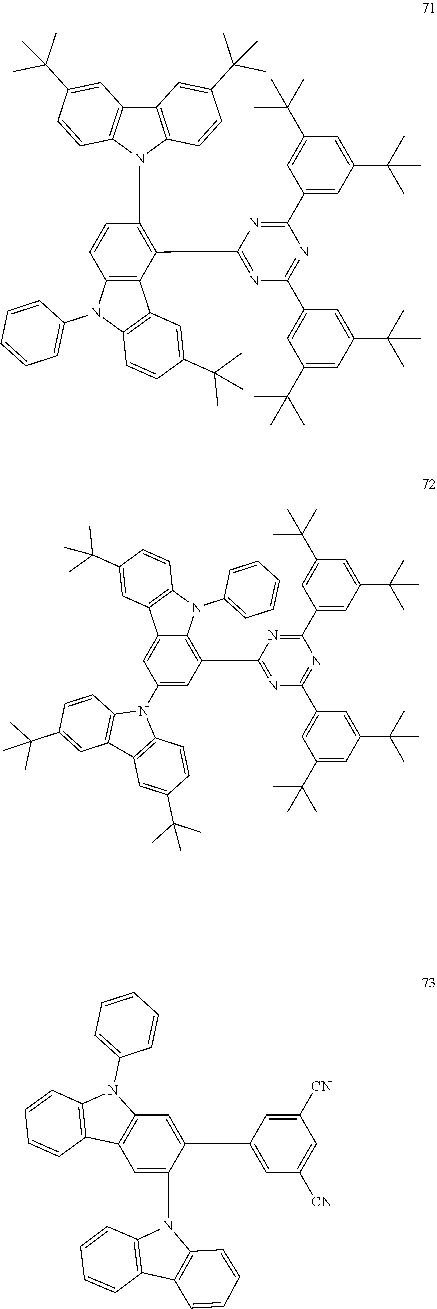 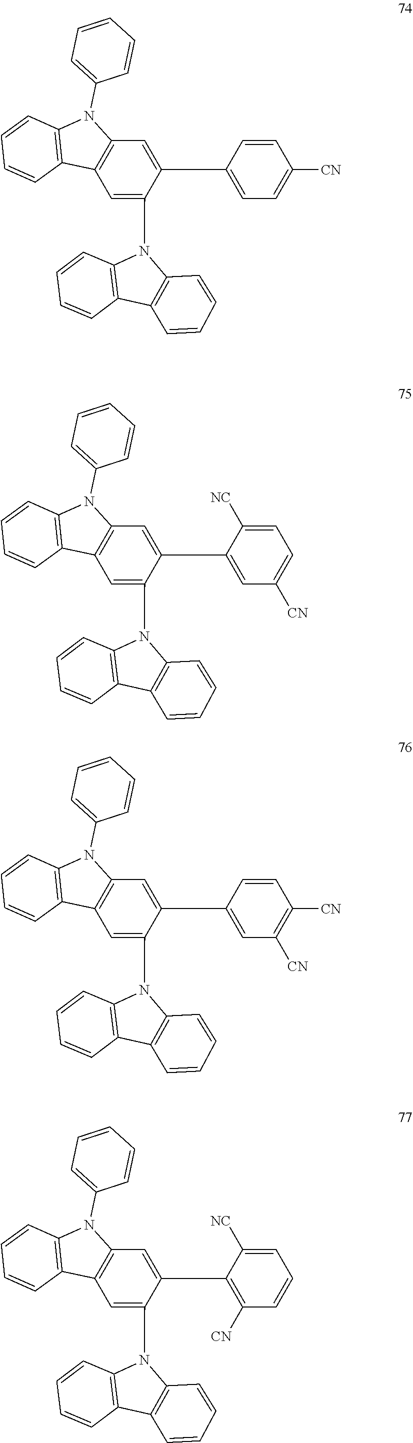 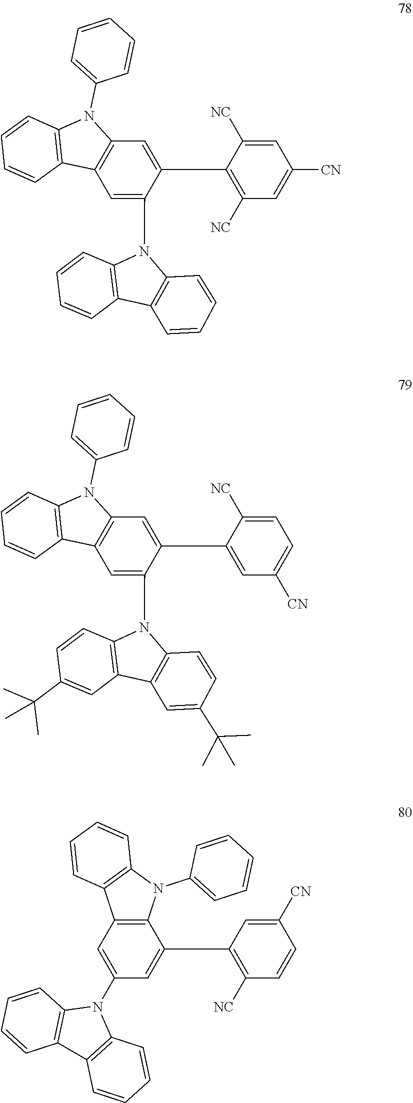 [SMILES: N#Cc1cc(C#N)c(-c2cc3c(cc2-n2c4ccccc4c4ccccc42)c2ccccc2n3-c2ccccc2)c(C#N)c1 ; CC(C)(C)c1ccc2c(c1)c1cc(C(C)(C)C)ccc1n2-c1cc2c3ccccc3n(-c3ccccc3)c2cc1-c1cc(C#N)ccc1C#N ; N#Cc1ccc(C#N)c(-c2cc(-n3c4ccccc4c4ccccc43)cc3c4ccccc4n(-c4ccccc4)c23)c1] 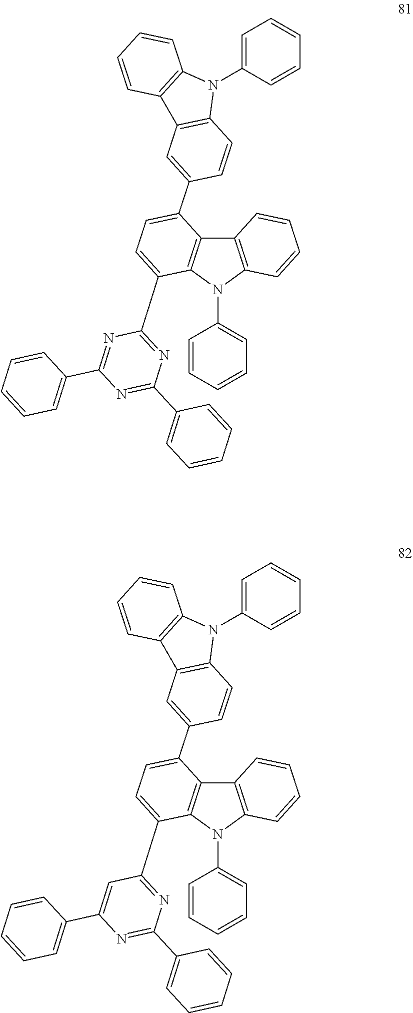 [SMILES: c1ccc(-c2nc(-c3ccccc3)nc(-c3ccc(-c4ccc5c(c4)c4ccccc4n5-c4ccccc4)c4c5ccccc5n(-c5ccccc5)c34)n2)cc1 ; c1ccc(-c2cc(-c3ccc(-c4ccc5c(c4)c4ccccc4n5-c4ccccc4)c4c5ccccc5n(-c5ccccc5)c34)nc(-c3ccccc3)n2)cc1] 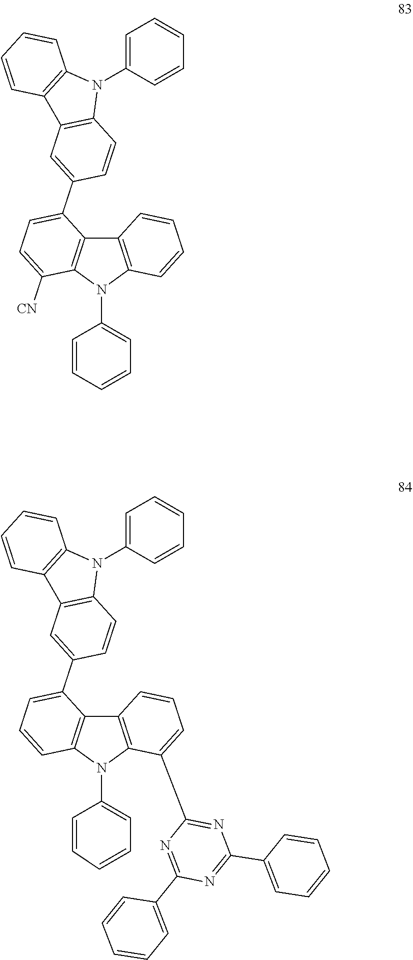 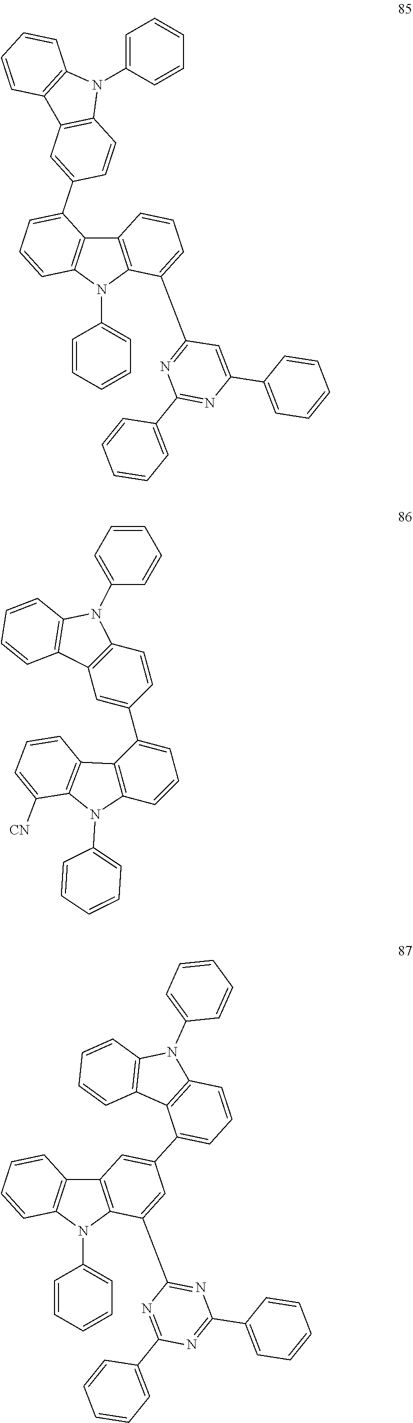 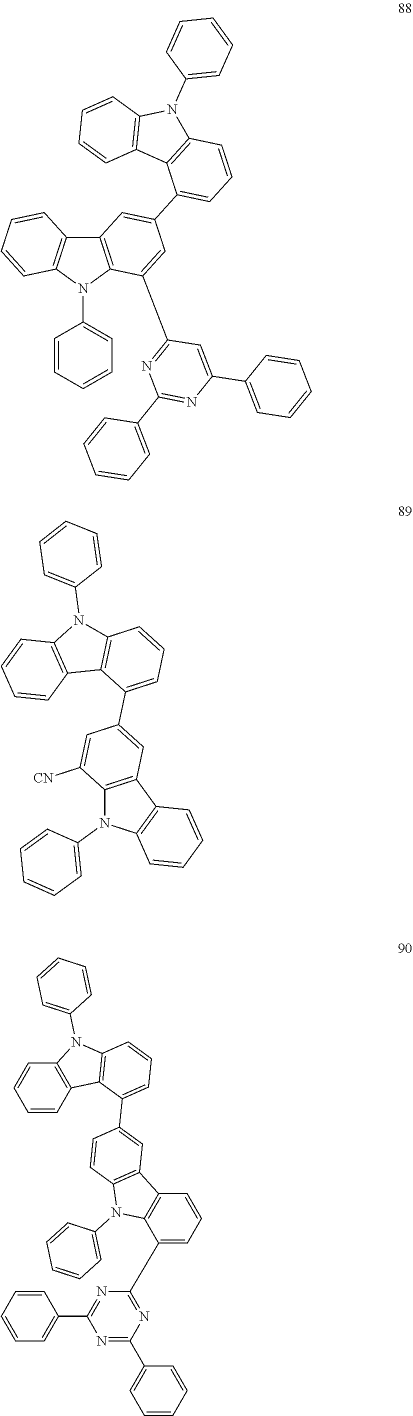 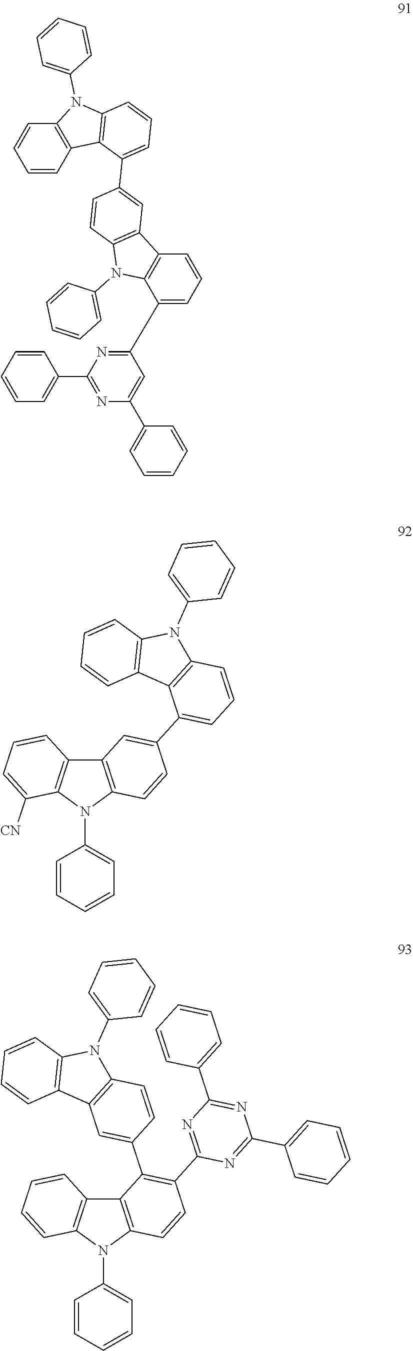 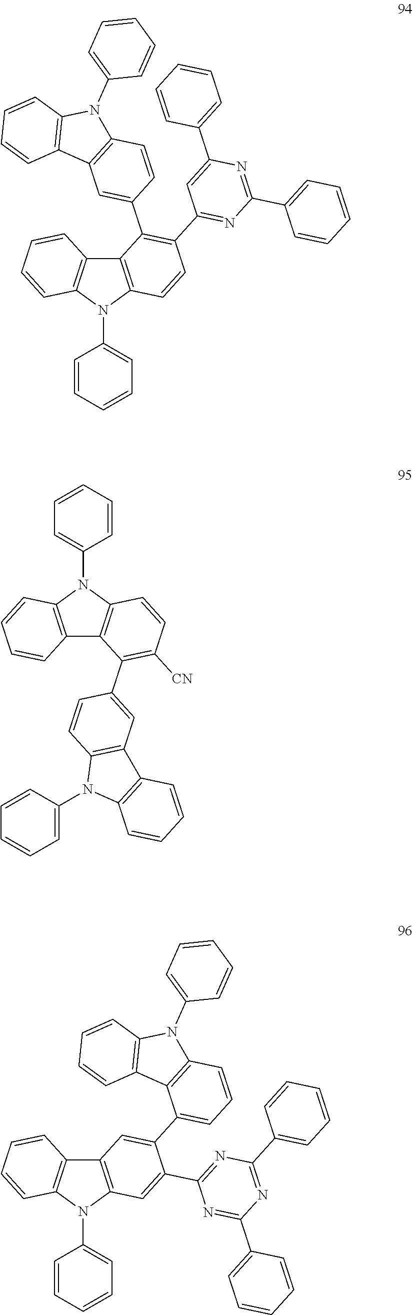 [SMILES: c1ccc(-c2cc(-c3ccc4c(c3-c3ccc5c(c3)c3ccccc3n5-c3ccccc3)c3ccccc3n4-c3ccccc3)nc(-c3ccccc3)n2)cc1 ; N#Cc1ccc2c(c1-c1ccc3c(c1)c1ccccc1n3-c1ccccc1)c1ccccc1n2-c1ccccc1 ; c1ccc(-c2nc(-c3ccccc3)nc(-c3cc4c(cc3-c3cccc5c3c3ccccc3n5-c3ccccc3)c3ccccc3n4-c3ccccc3)n2)cc1] 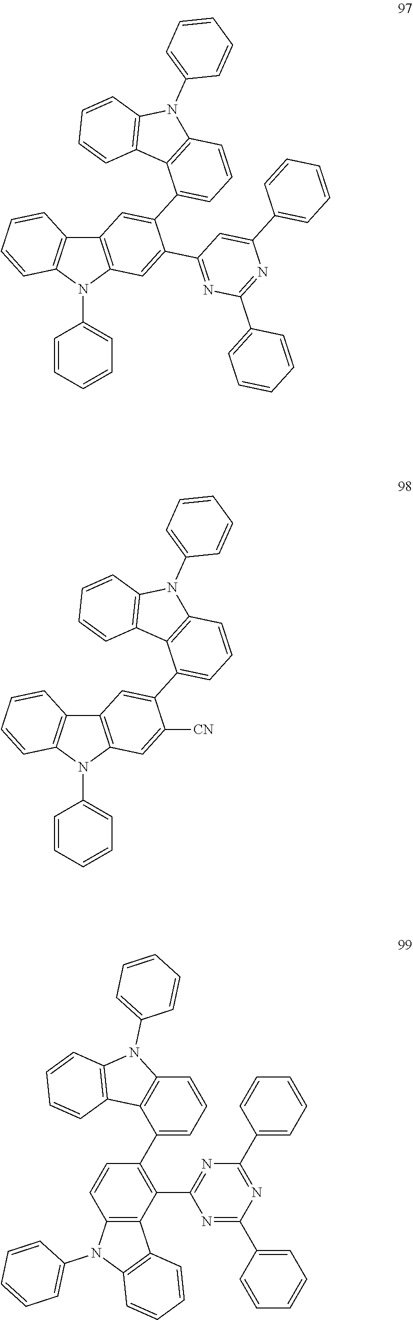 [SMILES: c1ccc(-c2cc(-c3cc4c(cc3-c3cccc5c3c3ccccc3n5-c3ccccc3)c3ccccc3n4-c3ccccc3)nc(-c3ccccc3)n2)cc1 ; N#Cc1cc2c(cc1-c1cccc3c1c1ccccc1n3-c1ccccc1)c1ccccc1n2-c1ccccc1 ; c1ccc(-c2nc(-c3ccccc3)nc(-c3c(-c4cccc5c4c4ccccc4n5-c4ccccc4)ccc4c3c3ccccc3n4-c3ccccc3)n2)cc1] 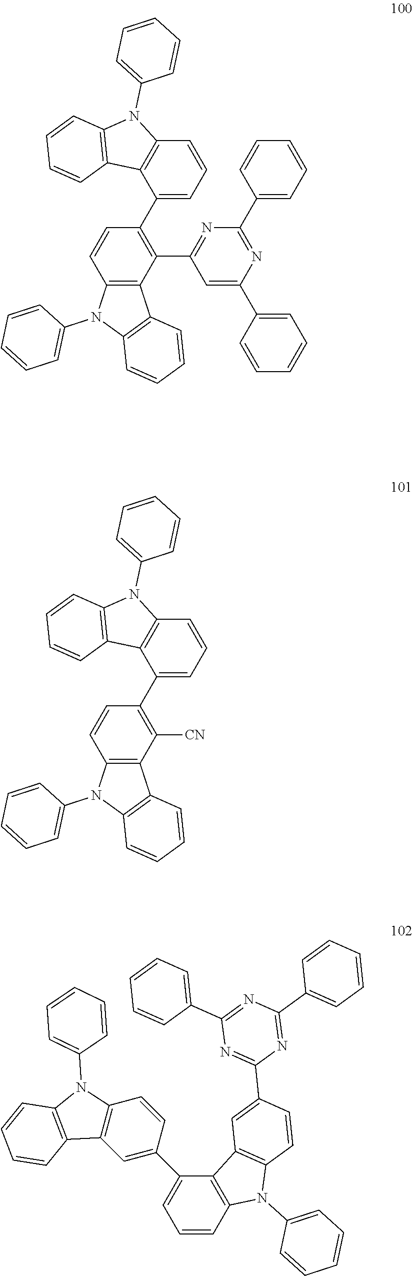 [SMILES: c1ccc(-c2cc(-c3c(-c4cccc5c4c4ccccc4n5-c4ccccc4)ccc4c3c3ccccc3n4-c3ccccc3)nc(-c3ccccc3)n2)cc1 ; N#Cc1c(-c2cccc3c2c2ccccc2n3-c2ccccc2)ccc2c1c1ccccc1n2-c1ccccc1 ; c1ccc(-c2nc(-c3ccccc3)nc(-c3ccc4c(c3)c3c(-c5ccc6c(c5)c5ccccc5n6-c5ccccc5)cccc3n4-c3ccccc3)n2)cc1] 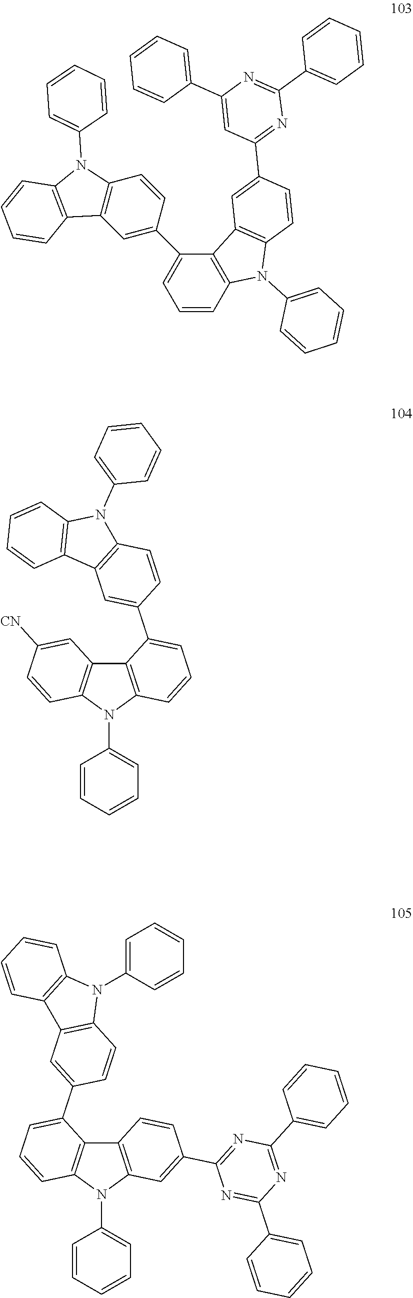 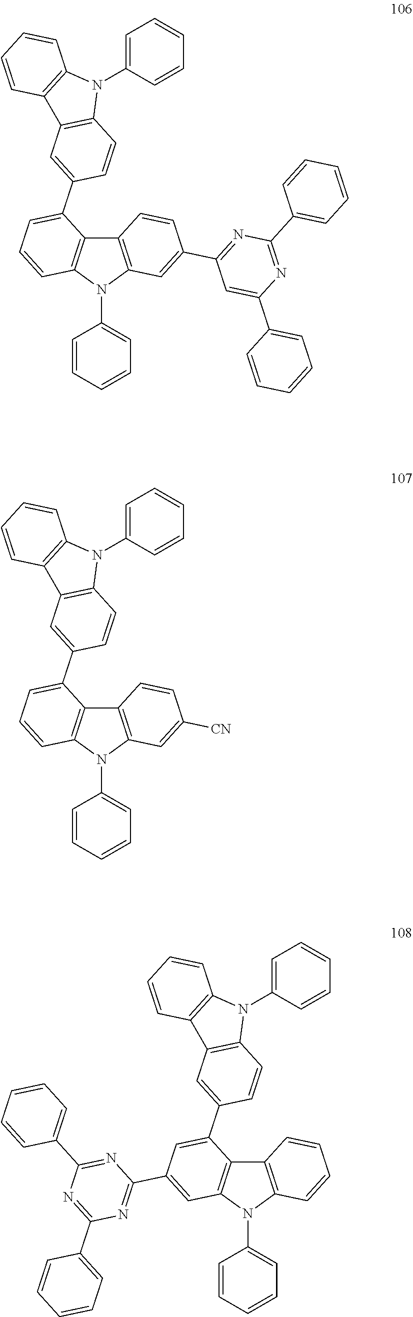 [SMILES: c1ccc(-c2cc(-c3ccc4c5c(-c6ccc7c(c6)c6ccccc6n7-c6ccccc6)cccc5n(-c5ccccc5)c4c3)nc(-c3ccccc3)n2)cc1 ; N#Cc1ccc2c3c(-c4ccc5c(c4)c4ccccc4n5-c4ccccc4)cccc3n(-c3ccccc3)c2c1 ; c1ccc(-c2nc(-c3ccccc3)nc(-c3cc(-c4ccc5c(c4)c4ccccc4n5-c4ccccc4)c4c5ccccc5n(-c5ccccc5)c4c3)n2)cc1] 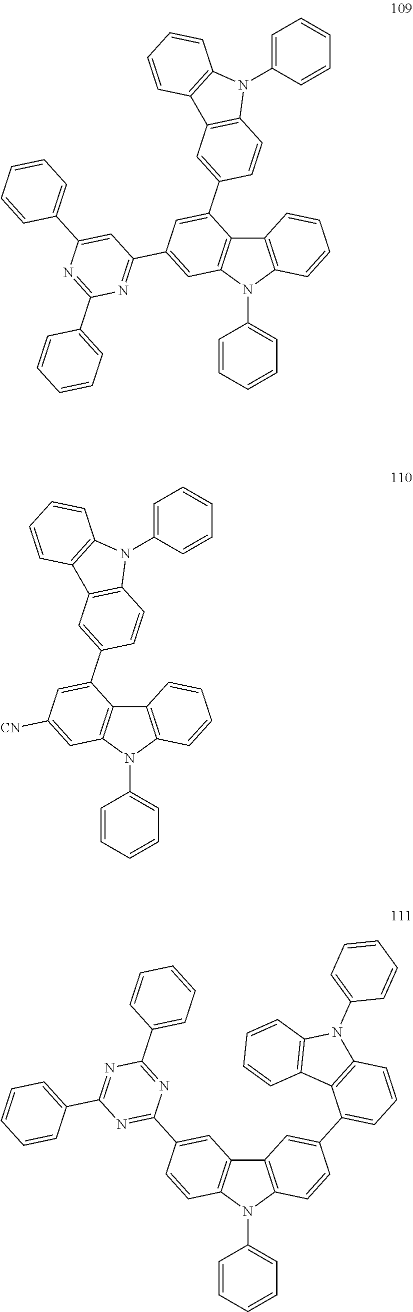 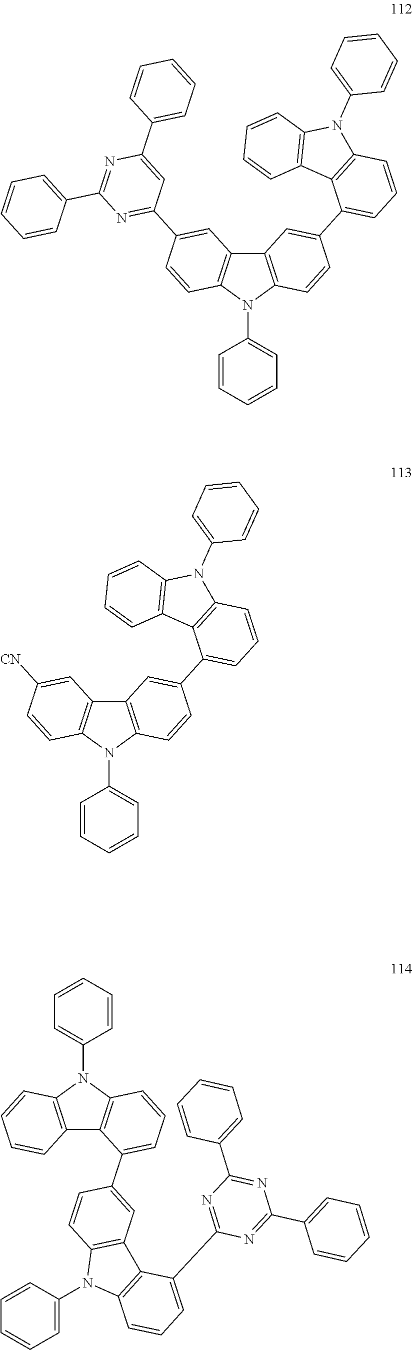 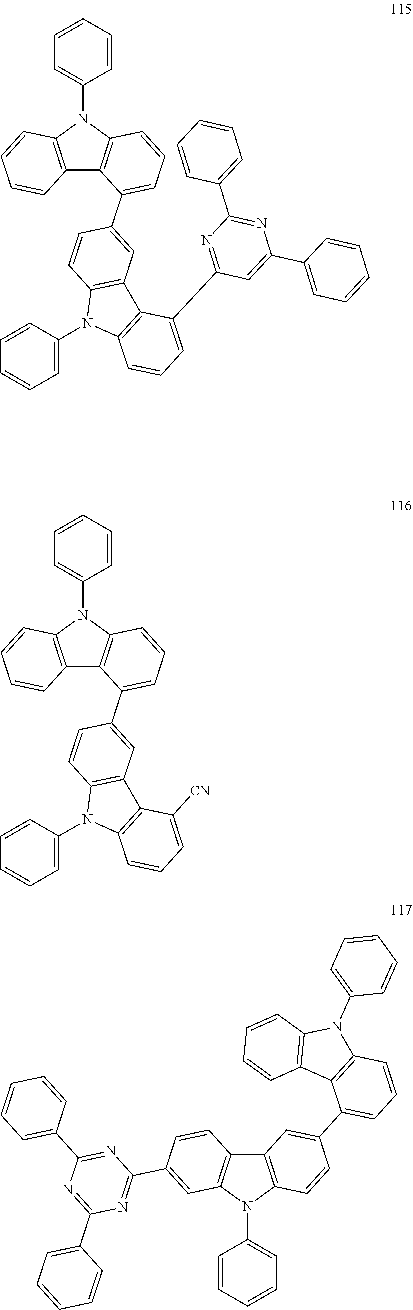 [SMILES: c1ccc(-c2cc(-c3cccc4c3c3cc(-c5cccc6c5c5ccccc5n6-c5ccccc5)ccc3n4-c3ccccc3)nc(-c3ccccc3)n2)cc1 ; N#Cc1cccc2c1c1cc(-c3cccc4c3c3ccccc3n4-c3ccccc3)ccc1n2-c1ccccc1 ; c1ccc(-c2nc(-c3ccccc3)nc(-c3ccc4c5cc(-c6cccc7c6c6ccccc6n7-c6ccccc6)ccc5n(-c5ccccc5)c4c3)n2)cc1] 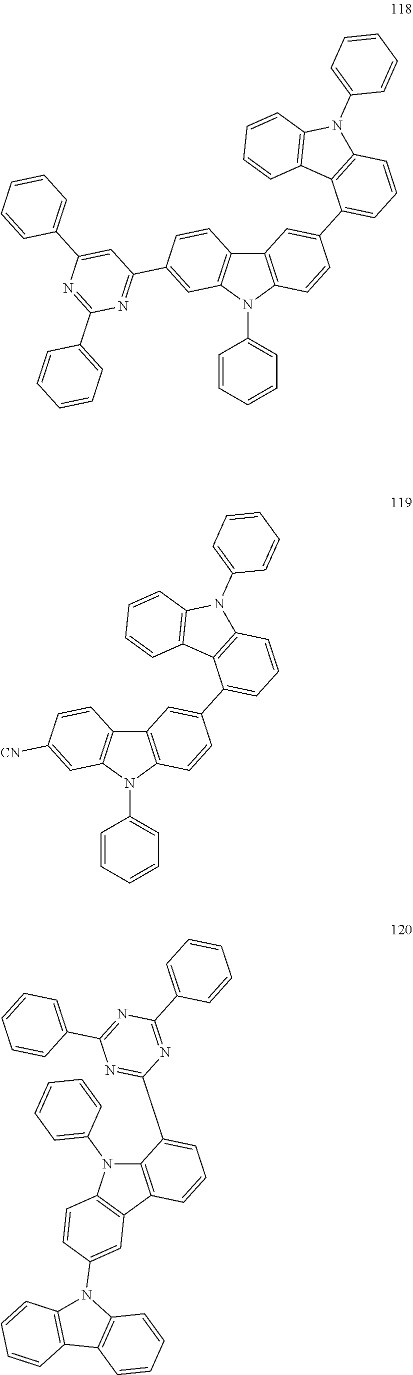 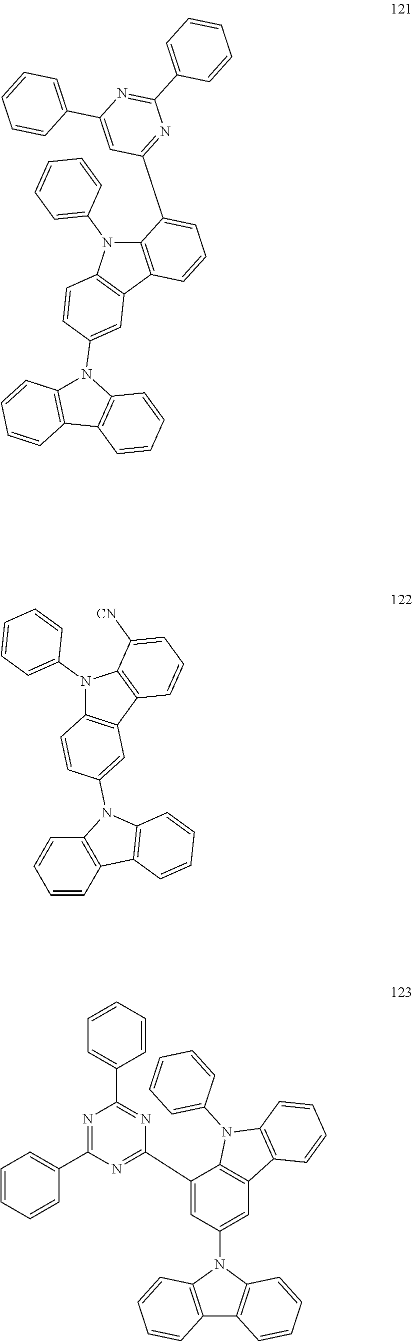 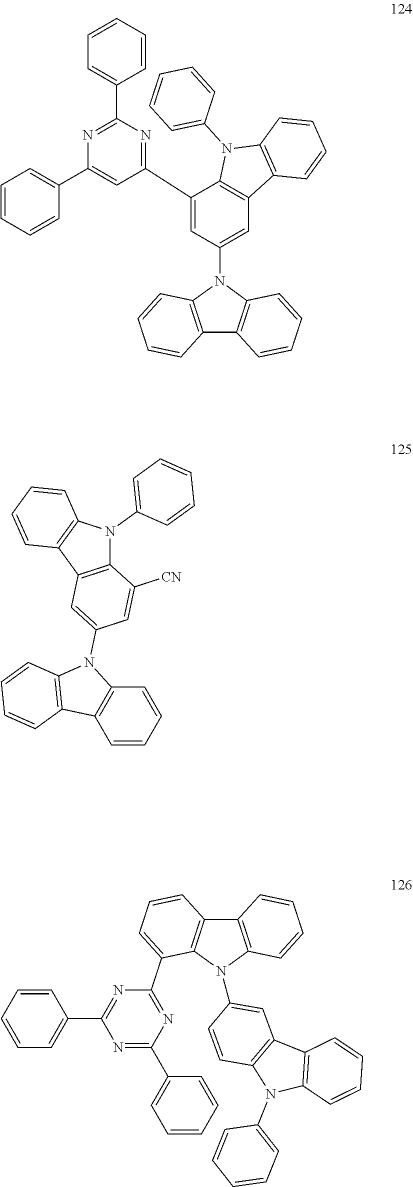 [SMILES: c1ccc(-c2cc(-c3cc(-n4c5ccccc5c5ccccc54)cc4c5ccccc5n(-c5ccccc5)c34)nc(-c3ccccc3)n2)cc1 ; N#Cc1cc(-n2c3ccccc3c3ccccc32)cc2c3ccccc3n(-c3ccccc3)c12 ; c1ccc(-c2nc(-c3ccccc3)nc(-c3cccc4c5ccccc5n(-c5ccc6c(c5)c5ccccc5n6-c5ccccc5)c34)n2)cc1] 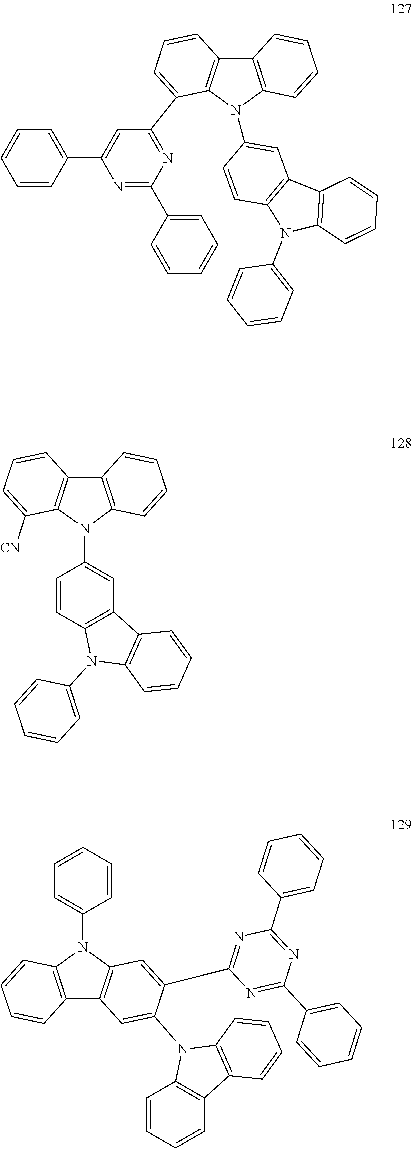 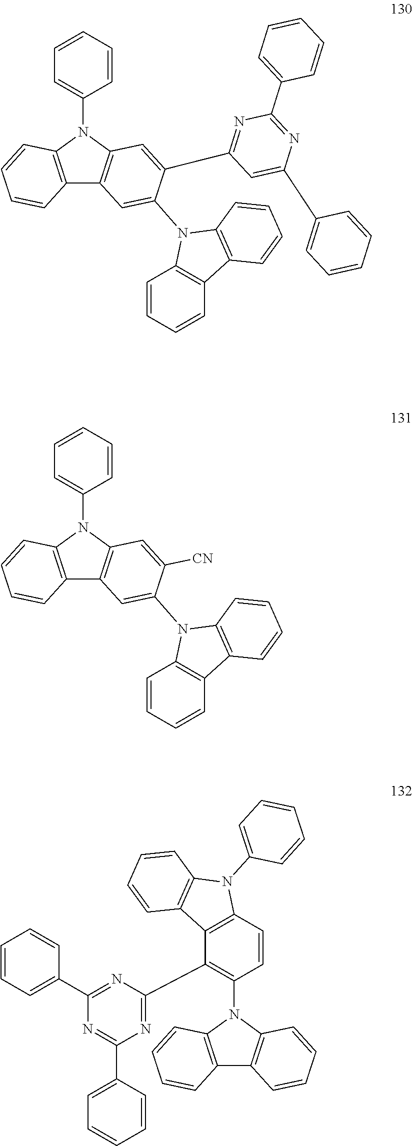 [SMILES: c1ccc(-c2cc(-c3cc4c(cc3-n3c5ccccc5c5ccccc53)c3ccccc3n4-c3ccccc3)nc(-c3ccccc3)n2)cc1 ; N#Cc1cc2c(cc1-n1c3ccccc3c3ccccc31)c1ccccc1n2-c1ccccc1 ; c1ccc(-c2nc(-c3ccccc3)nc(-c3c(-n4c5ccccc5c5ccccc54)ccc4c3c3ccccc3n4-c3ccccc3)n2)cc1] 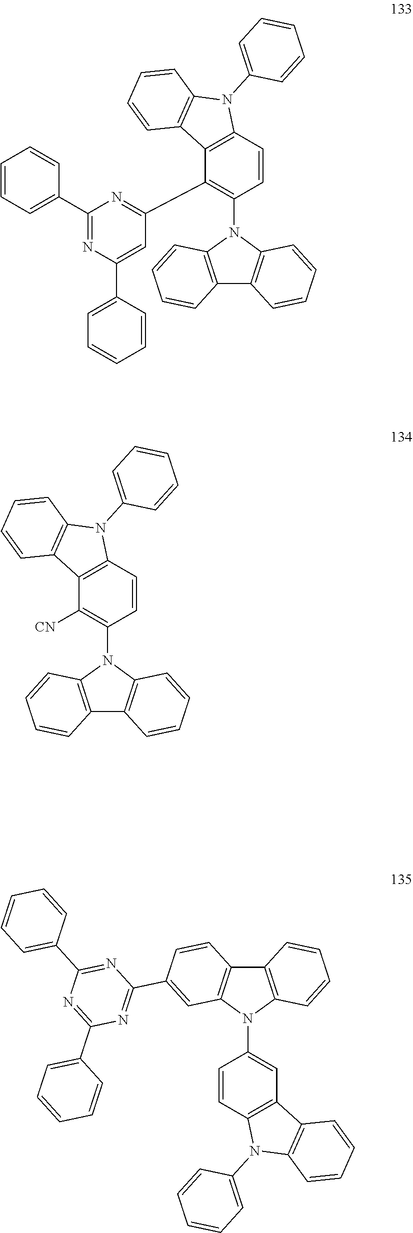 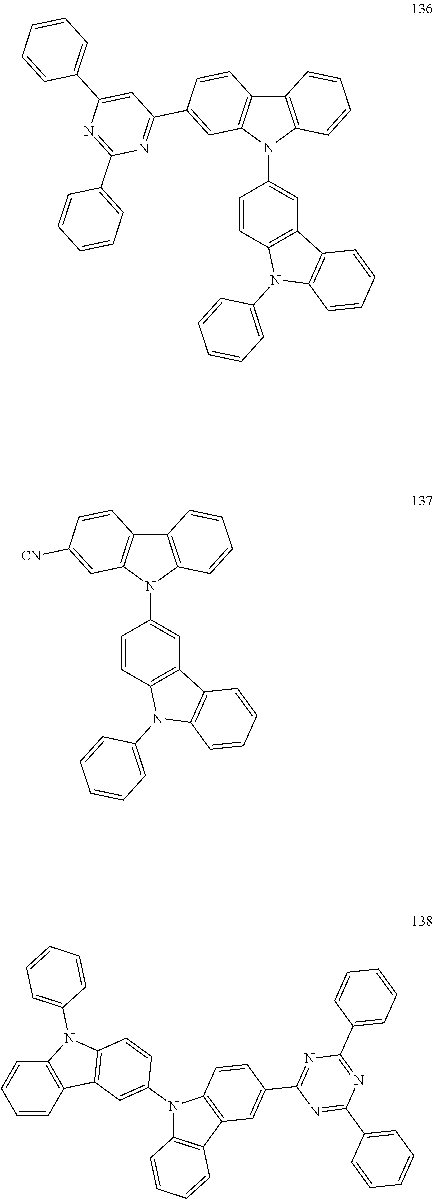 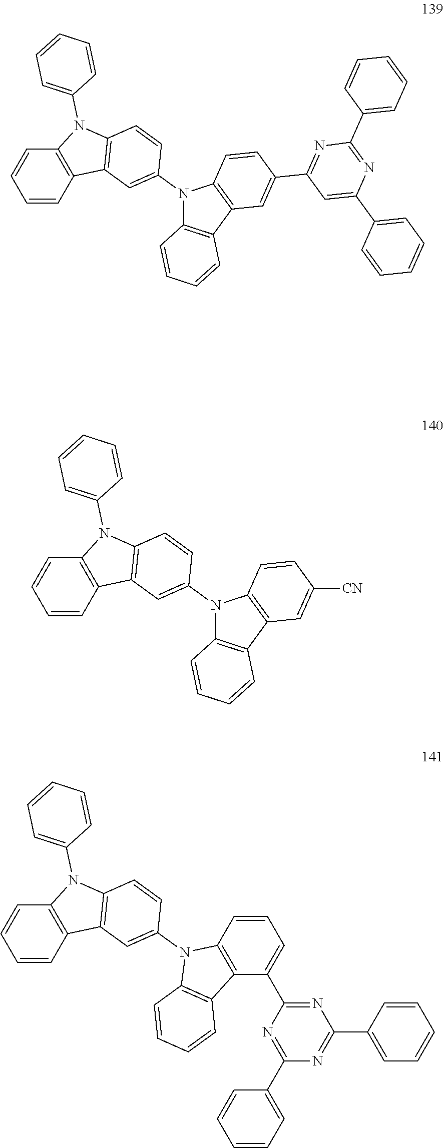 [SMILES: c1ccc(-c2cc(-c3ccc4c(c3)c3ccccc3n4-c3ccc4c(c3)c3ccccc3n4-c3ccccc3)nc(-c3ccccc3)n2)cc1 ; N#Cc1ccc2c(c1)c1ccccc1n2-c1ccc2c(c1)c1ccccc1n2-c1ccccc1 ; c1ccc(-c2nc(-c3ccccc3)nc(-c3cccc4c3c3ccccc3n4-c3ccc4c(c3)c3ccccc3n4-c3ccccc3)n2)cc1] 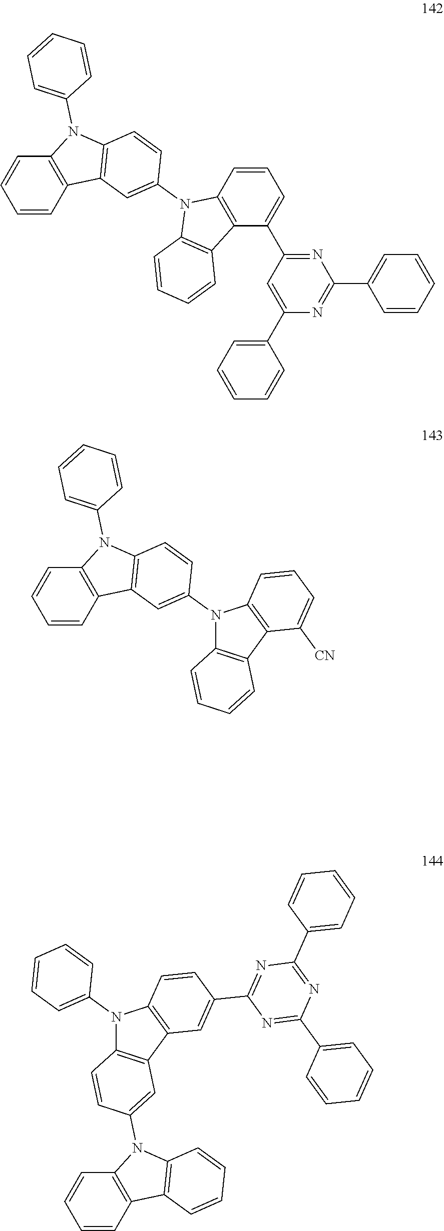 [SMILES: c1ccc(-c2cc(-c3cccc4c3c3ccccc3n4-c3ccc4c(c3)c3ccccc3n4-c3ccccc3)nc(-c3ccccc3)n2)cc1 ; N#Cc1cccc2c1c1ccccc1n2-c1ccc2c(c1)c1ccccc1n2-c1ccccc1 ; c1ccc(-c2nc(-c3ccccc3)nc(-c3ccc4c(c3)c3cc(-n5c6ccccc6c6ccccc65)ccc3n4-c3ccccc3)n2)cc1] 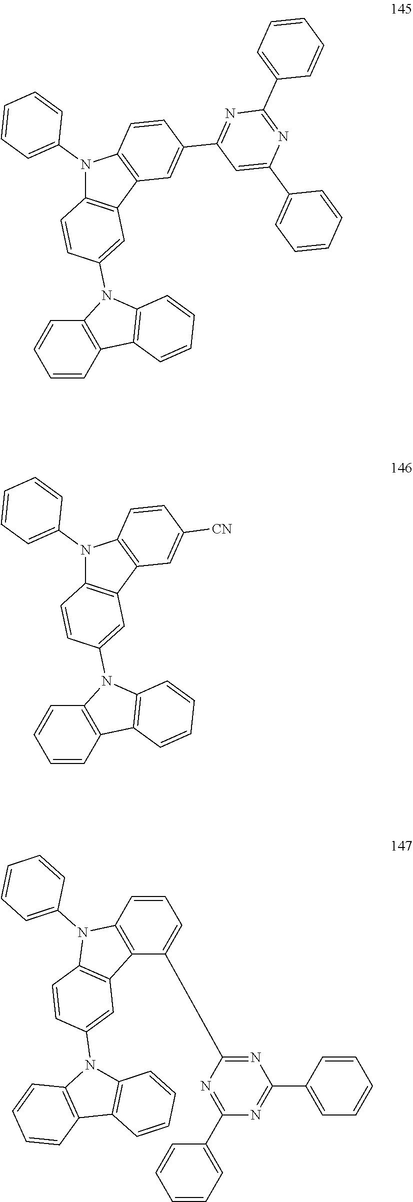 [SMILES: c1ccc(-c2cc(-c3ccc4c(c3)c3cc(-n5c6ccccc6c6ccccc65)ccc3n4-c3ccccc3)nc(-c3ccccc3)n2)cc1 ; N#Cc1ccc2c(c1)c1cc(-n3c4ccccc4c4ccccc43)ccc1n2-c1ccccc1 ; c1ccc(-c2nc(-c3ccccc3)nc(-c3cccc4c3c3cc(-n5c6ccccc6c6ccccc65)ccc3n4-c3ccccc3)n2)cc1] 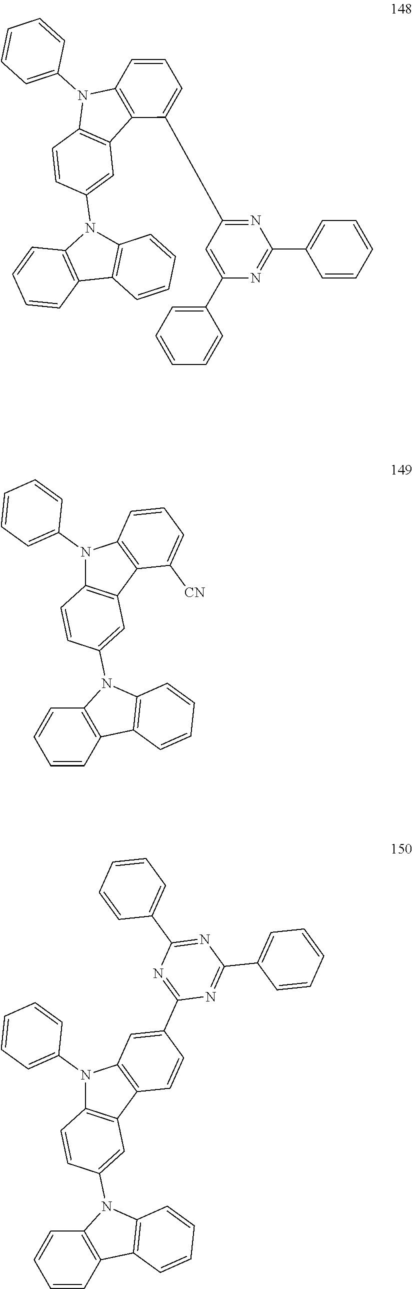 [SMILES: c1ccc(-c2cc(-c3cccc4c3c3cc(-n5c6ccccc6c6ccccc65)ccc3n4-c3ccccc3)nc(-c3ccccc3)n2)cc1 ; N#Cc1cccc2c1c1cc(-n3c4ccccc4c4ccccc43)ccc1n2-c1ccccc1 ; c1ccc(-c2nc(-c3ccccc3)nc(-c3ccc4c5cc(-n6c7ccccc7c7ccccc76)ccc5n(-c5ccccc5)c4c3)n2)cc1] 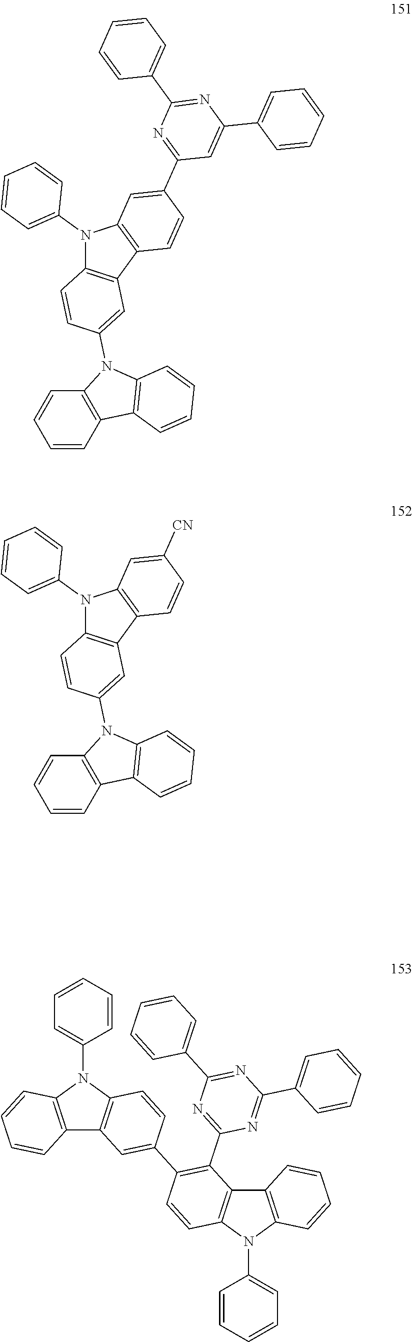 [SMILES: c1ccc(-c2cc(-c3ccc4c5cc(-n6c7ccccc7c7ccccc76)ccc5n(-c5ccccc5)c4c3)nc(-c3ccccc3)n2)cc1 ; N#Cc1ccc2c3cc(-n4c5ccccc5c5ccccc54)ccc3n(-c3ccccc3)c2c1 ; c1ccc(-c2nc(-c3ccccc3)nc(-c3c(-c4ccc5c(c4)c4ccccc4n5-c4ccccc4)ccc4c3c3ccccc3n4-c3ccccc3)n2)cc1] 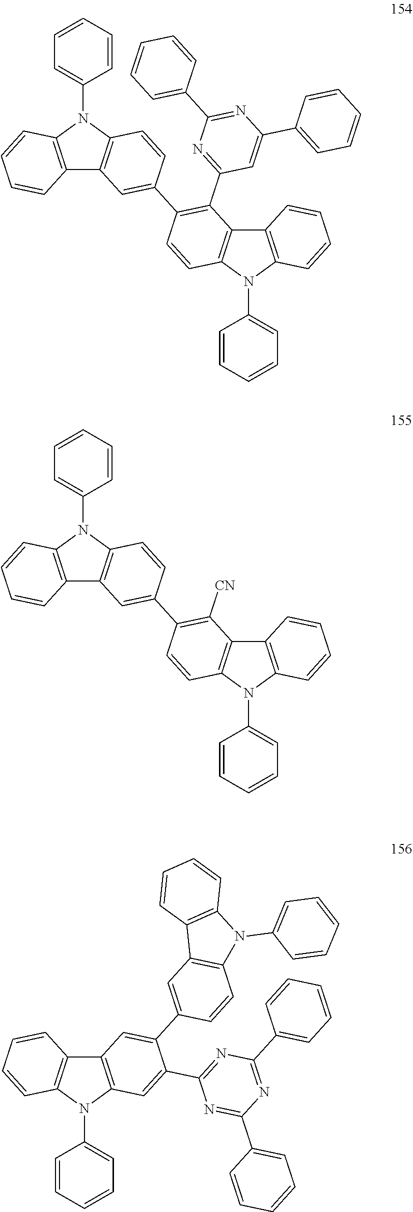 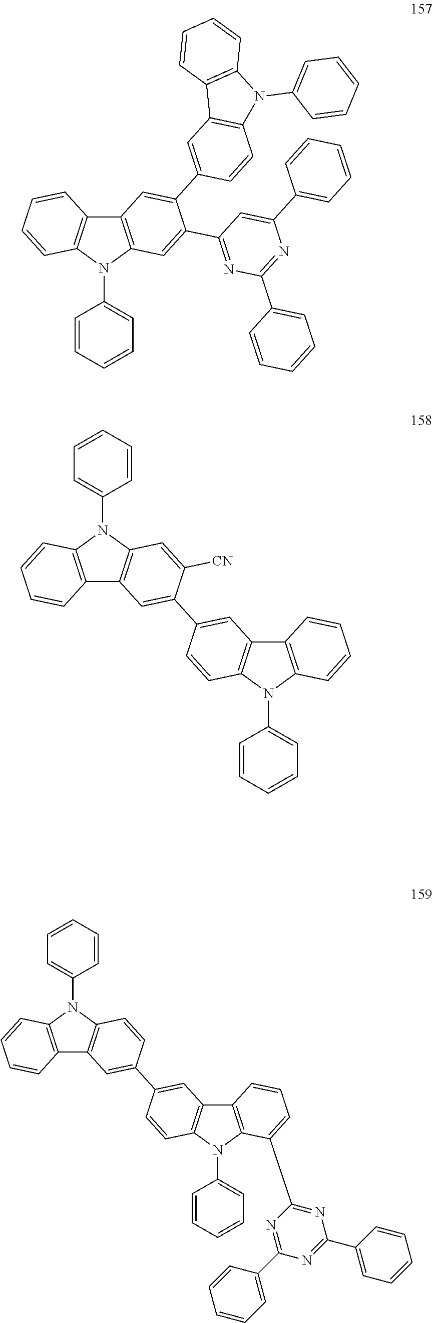 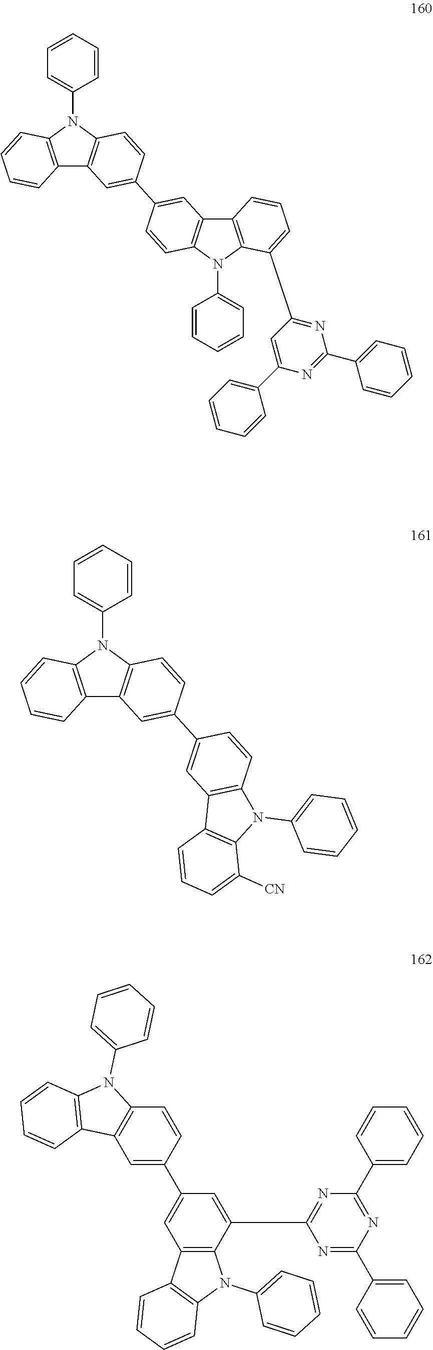 [SMILES: c1ccc(-c2cc(-c3cccc4c5cc(-c6ccc7c(c6)c6ccccc6n7-c6ccccc6)ccc5n(-c5ccccc5)c34)nc(-c3ccccc3)n2)cc1 ; N#Cc1cccc2c3cc(-c4ccc5c(c4)c4ccccc4n5-c4ccccc4)ccc3n(-c3ccccc3)c12 ; c1ccc(-c2nc(-c3ccccc3)nc(-c3cc(-c4ccc5c(c4)c4ccccc4n5-c4ccccc4)cc4c5ccccc5n(-c5ccccc5)c34)n2)cc1] 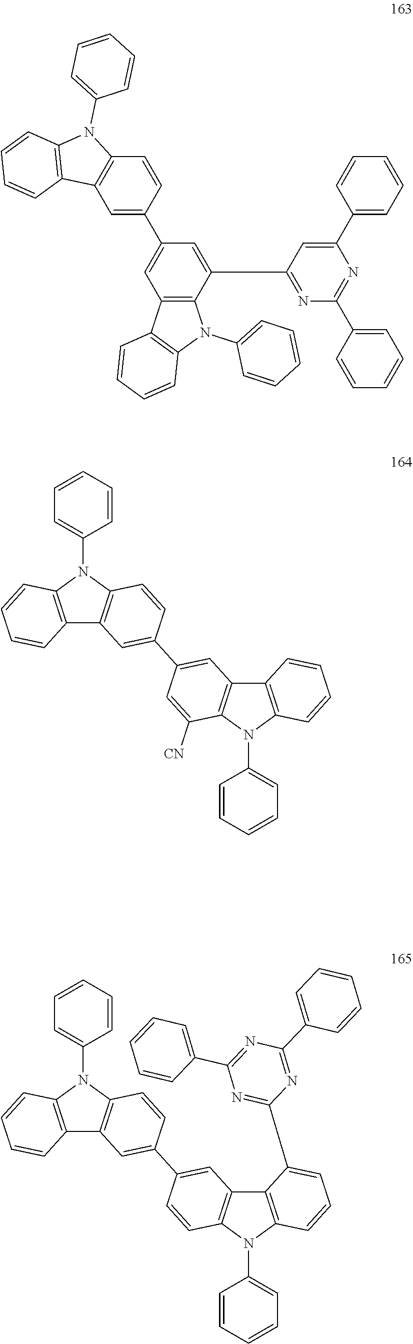 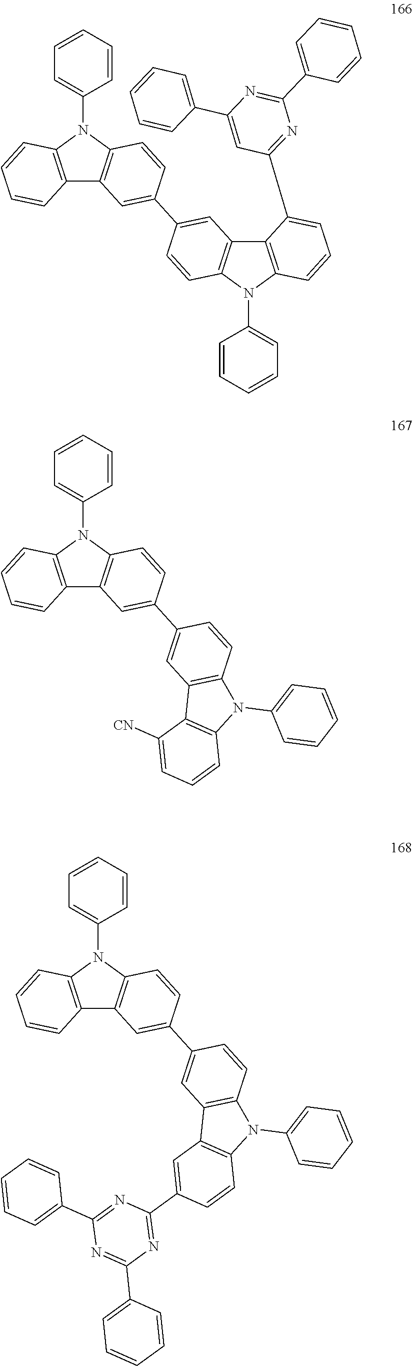 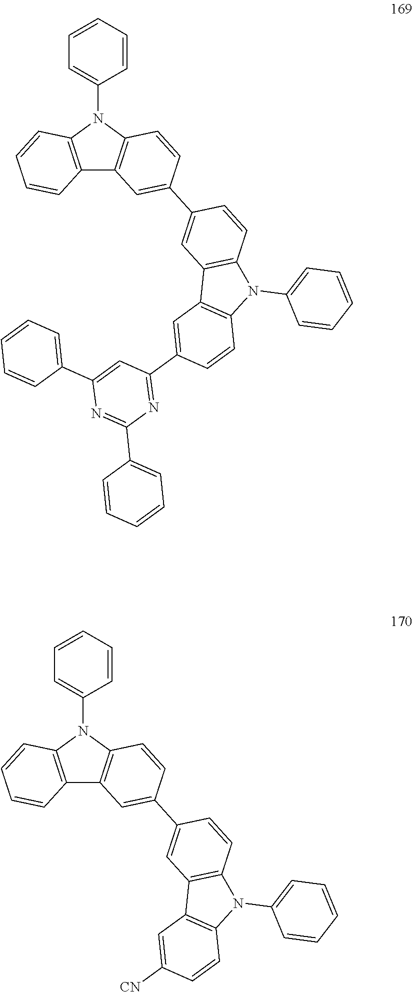 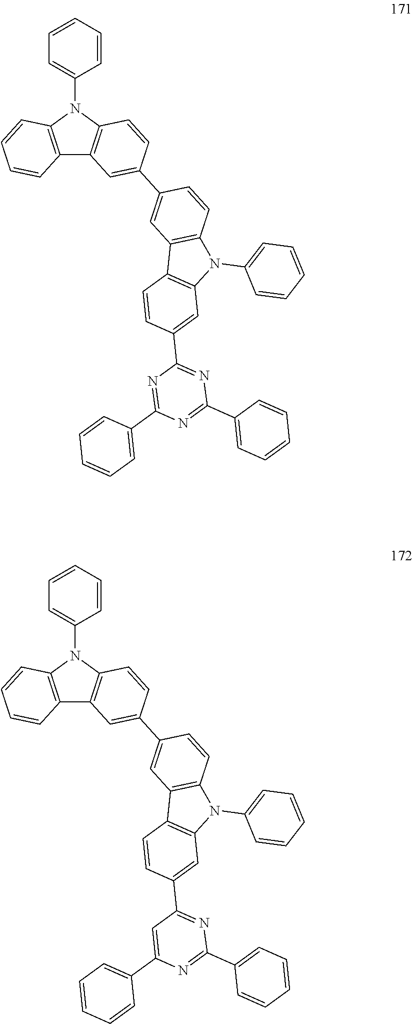 [SMILES: c1ccc(-c2nc(-c3ccccc3)nc(-c3ccc4c5cc(-c6ccc7c(c6)c6ccccc6n7-c6ccccc6)ccc5n(-c5ccccc5)c4c3)n2)cc1 ; c1ccc(-c2cc(-c3ccc4c5cc(-c6ccc7c(c6)c6ccccc6n7-c6ccccc6)ccc5n(-c5ccccc5)c4c3)nc(-c3ccccc3)n2)cc1] 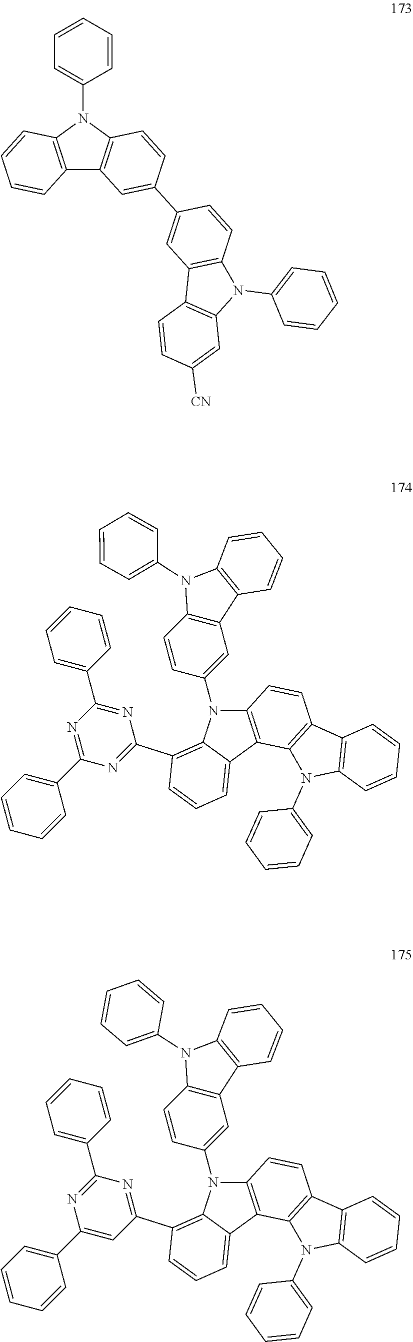 [SMILES: N#Cc1ccc2c3cc(-c4ccc5c(c4)c4ccccc4n5-c4ccccc4)ccc3n(-c3ccccc3)c2c1 ; c1ccc(-c2nc(-c3ccccc3)nc(-c3cccc4c5c(ccc6c7ccccc7n(-c7ccccc7)c65)n(-c5ccc6c(c5)c5ccccc5n6-c5ccccc5)c34)n2)cc1 ; c1ccc(-c2cc(-c3cccc4c5c(ccc6c7ccccc7n(-c7ccccc7)c65)n(-c5ccc6c(c5)c5ccccc5n6-c5ccccc5)c34)nc(-c3ccccc3)n2)cc1] 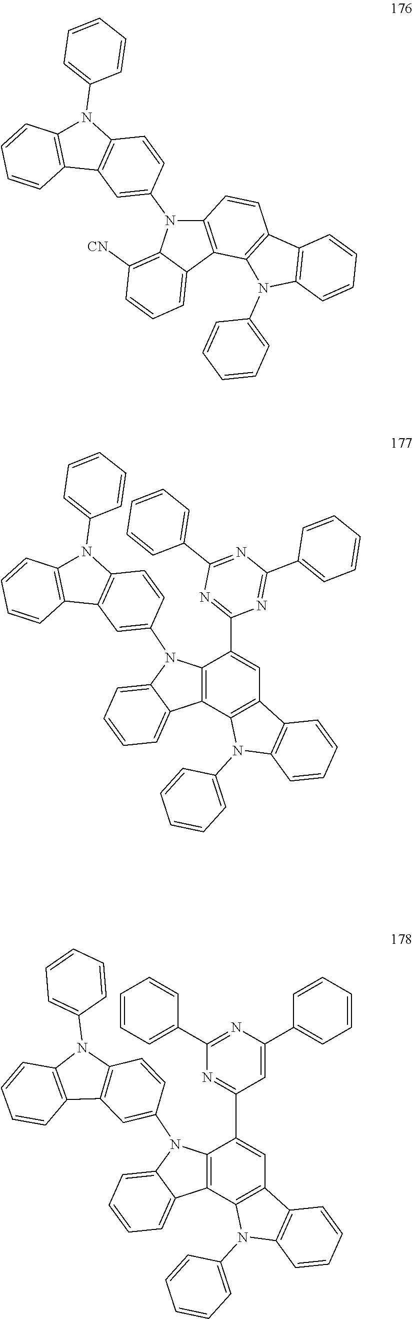 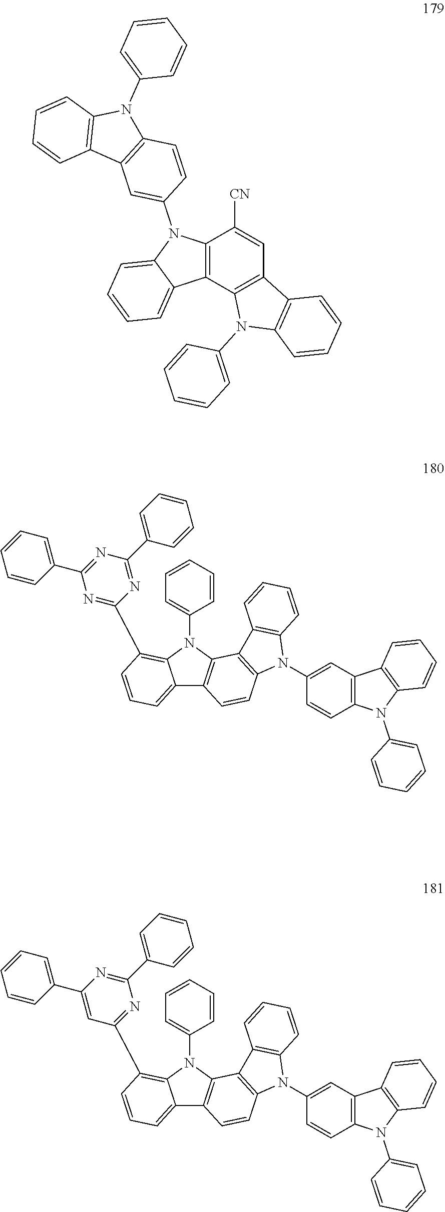 [SMILES: N#Cc1cc2c3ccccc3n(-c3ccccc3)c2c2c3ccccc3n(-c3ccc4c(c3)c3ccccc3n4-c3ccccc3)c12 ; c1ccc(-c2nc(-c3ccccc3)nc(-c3cccc4c5ccc6c(c7ccccc7n6-c6ccc7c(c6)c6ccccc6n7-c6ccccc6)c5n(-c5ccccc5)c34)n2)cc1 ; c1ccc(-c2cc(-c3cccc4c5ccc6c(c7ccccc7n6-c6ccc7c(c6)c6ccccc6n7-c6ccccc6)c5n(-c5ccccc5)c34)nc(-c3ccccc3)n2)cc1] 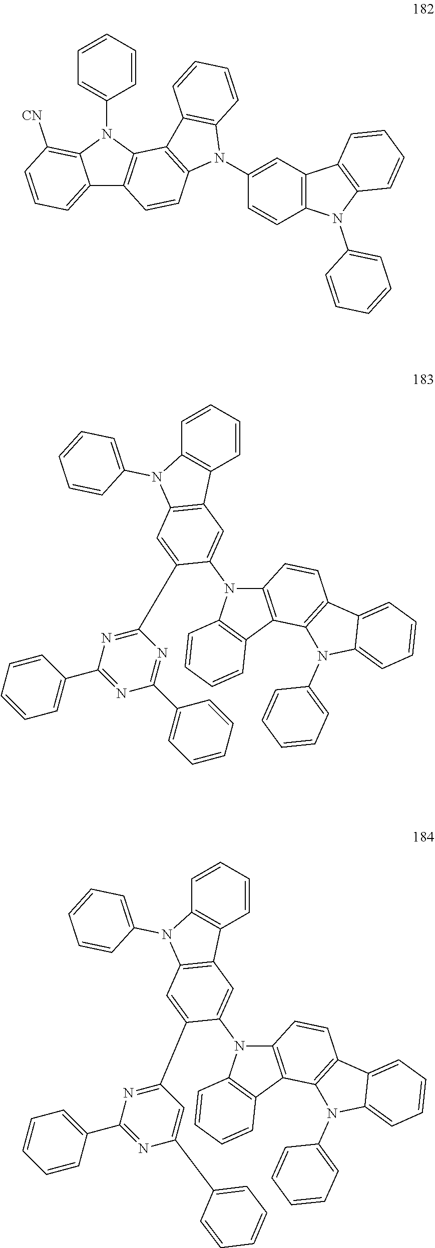 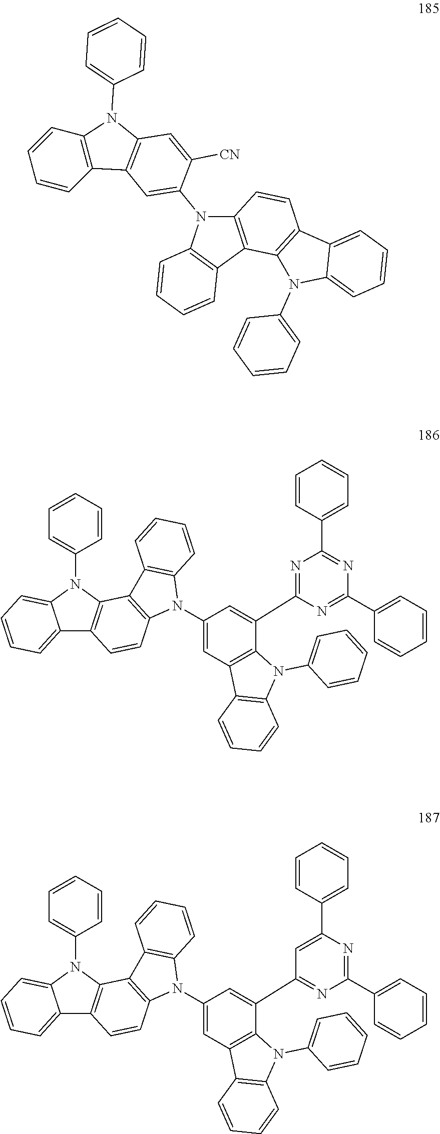 [SMILES: N#Cc1cc2c(cc1-n1c3ccccc3c3c1ccc1c4ccccc4n(-c4ccccc4)c13)c1ccccc1n2-c1ccccc1 ; c1ccc(-c2nc(-c3ccccc3)nc(-c3cc(-n4c5ccccc5c5c4ccc4c6ccccc6n(-c6ccccc6)c45)cc4c5ccccc5n(-c5ccccc5)c34)n2)cc1 ; c1ccc(-c2cc(-c3cc(-n4c5ccccc5c5c4ccc4c6ccccc6n(-c6ccccc6)c45)cc4c5ccccc5n(-c5ccccc5)c34)nc(-c3ccccc3)n2)cc1] 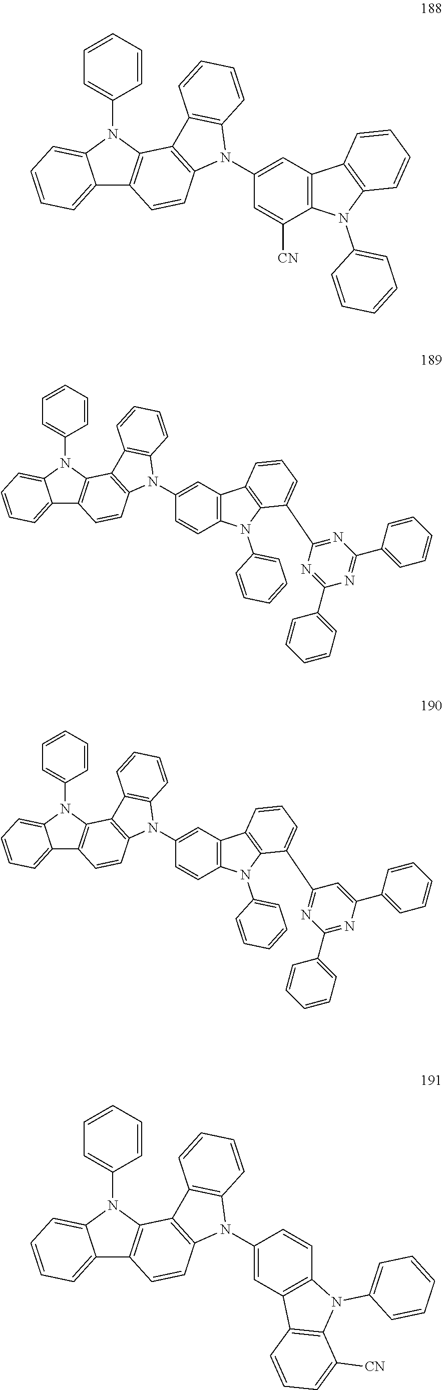 [SMILES: N#Cc1cc(-n2c3ccccc3c3c2ccc2c4ccccc4n(-c4ccccc4)c23)cc2c3ccccc3n(-c3ccccc3)c12 ; c1ccc(-c2nc(-c3ccccc3)nc(-c3cccc4c5cc(-n6c7ccccc7c7c6ccc6c8ccccc8n(-c8ccccc8)c67)ccc5n(-c5ccccc5)c34)n2)cc1 ; c1ccc(-c2cc(-c3cccc4c5cc(-n6c7ccccc7c7c6ccc6c8ccccc8n(-c8ccccc8)c67)ccc5n(-c5ccccc5)c34)nc(-c3ccccc3)n2)cc1 ; N#Cc1cccc2c3cc(-n4c5ccccc5c5c4ccc4c6ccccc6n(-c6ccccc6)c45)ccc3n(-c3ccccc3)c12] 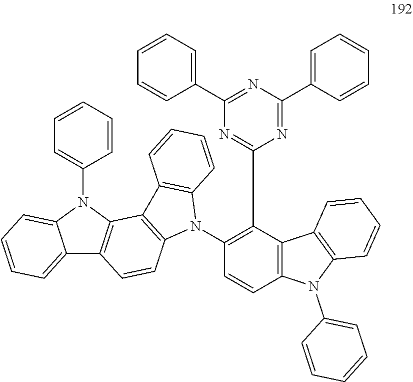 [SMILES: c1ccc(-c2nc(-c3ccccc3)nc(-c3c(-n4c5ccccc5c5c4ccc4c6ccccc6n(-c6ccccc6)c45)ccc4c3c3ccccc3n4-c3ccccc3)n2)cc1] 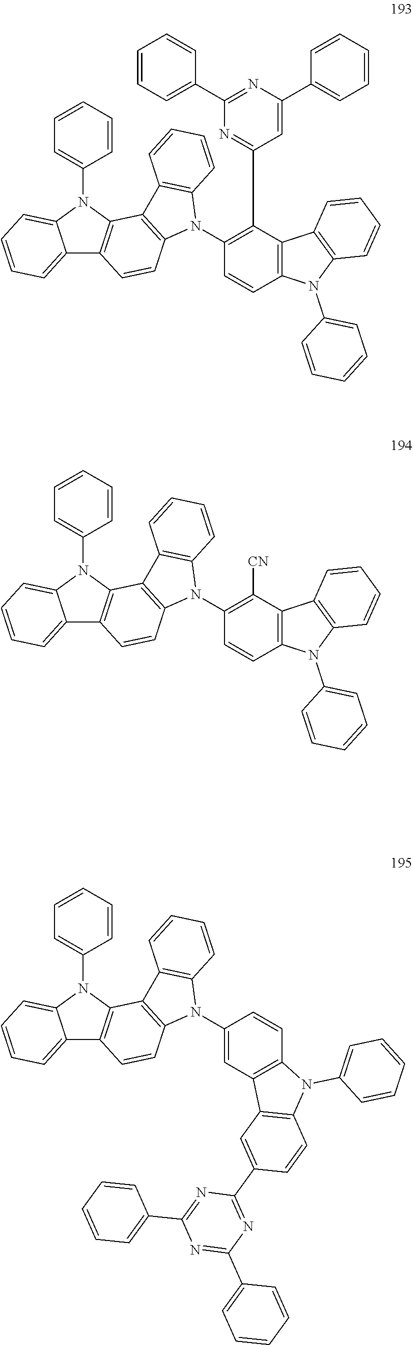 [SMILES: c1ccc(-c2cc(-c3c(-n4c5ccccc5c5c4ccc4c6ccccc6n(-c6ccccc6)c45)ccc4c3c3ccccc3n4-c3ccccc3)nc(-c3ccccc3)n2)cc1 ; N#Cc1c(-n2c3ccccc3c3c2ccc2c4ccccc4n(-c4ccccc4)c23)ccc2c1c1ccccc1n2-c1ccccc1 ; c1ccc(-c2nc(-c3ccccc3)nc(-c3ccc4c(c3)c3cc(-n5c6ccccc6c6c5ccc5c7ccccc7n(-c7ccccc7)c56)ccc3n4-c3ccccc3)n2)cc1] 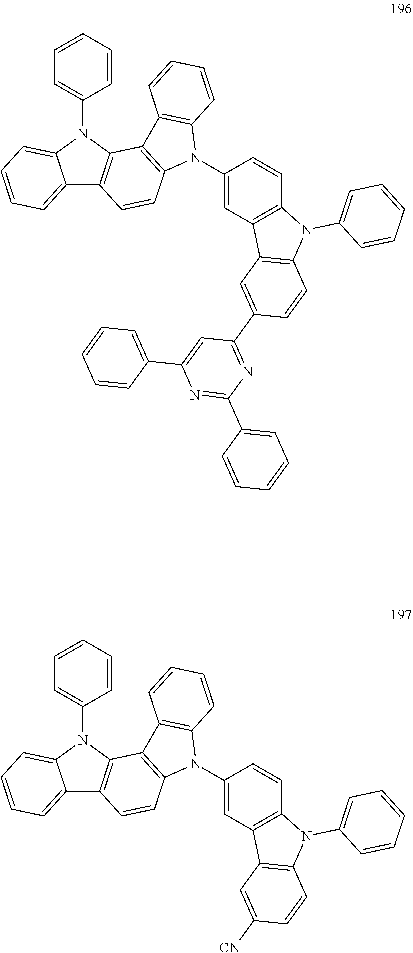 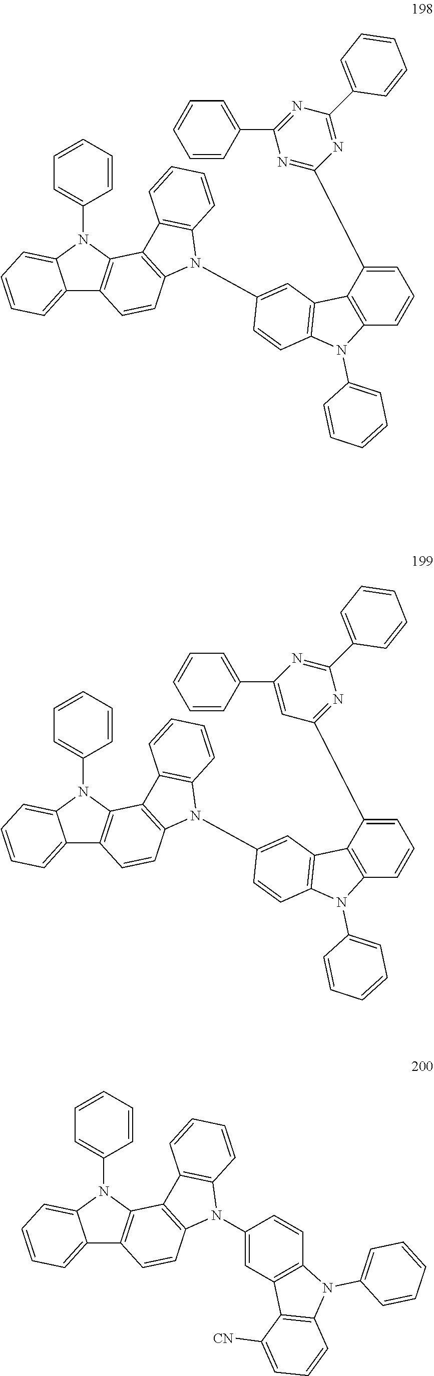 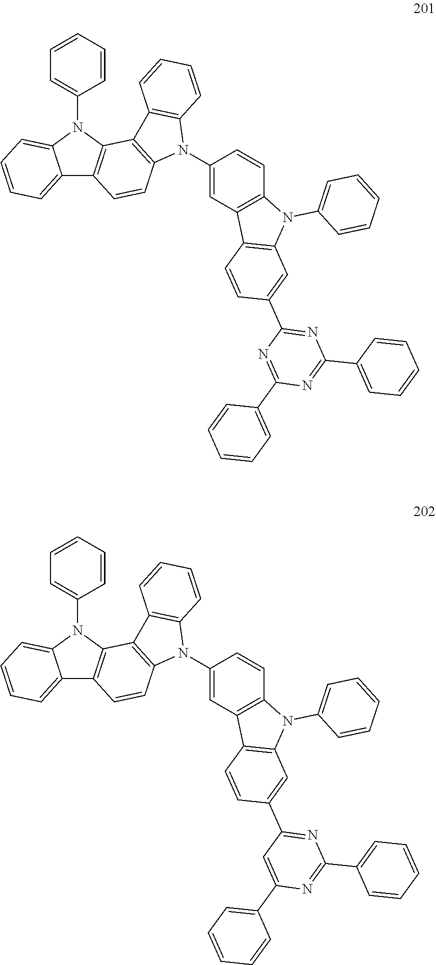 [SMILES: c1ccc(-c2nc(-c3ccccc3)nc(-c3ccc4c5cc(-n6c7ccccc7c7c6ccc6c8ccccc8n(-c8ccccc8)c67)ccc5n(-c5ccccc5)c4c3)n2)cc1 ; c1ccc(-c2cc(-c3ccc4c5cc(-n6c7ccccc7c7c6ccc6c8ccccc8n(-c8ccccc8)c67)ccc5n(-c5ccccc5)c4c3)nc(-c3ccccc3)n2)cc1] 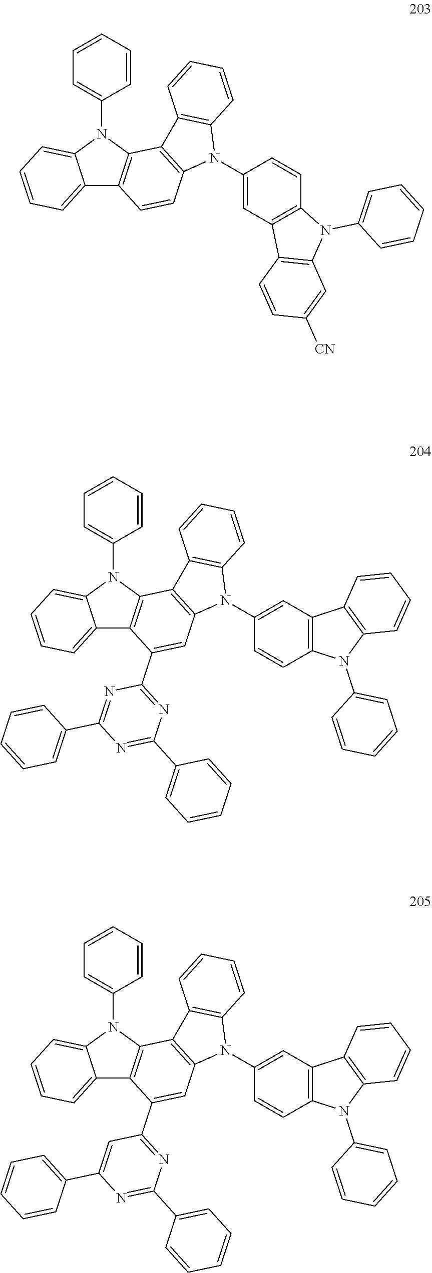 [SMILES: N#Cc1ccc2c3cc(-n4c5ccccc5c5c4ccc4c6ccccc6n(-c6ccccc6)c45)ccc3n(-c3ccccc3)c2c1 ; c1ccc(-c2nc(-c3ccccc3)nc(-c3cc4c(c5ccccc5n4-c4ccc5c(c4)c4ccccc4n5-c4ccccc4)c4c3c3ccccc3n4-c3ccccc3)n2)cc1 ; c1ccc(-c2cc(-c3cc4c(c5ccccc5n4-c4ccc5c(c4)c4ccccc4n5-c4ccccc4)c4c3c3ccccc3n4-c3ccccc3)nc(-c3ccccc3)n2)cc1] 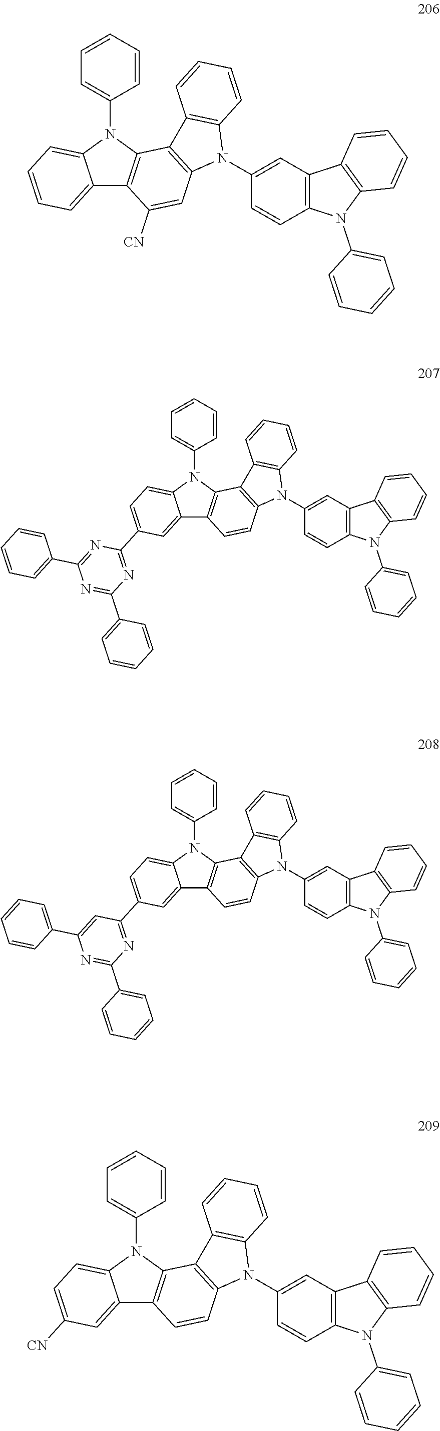 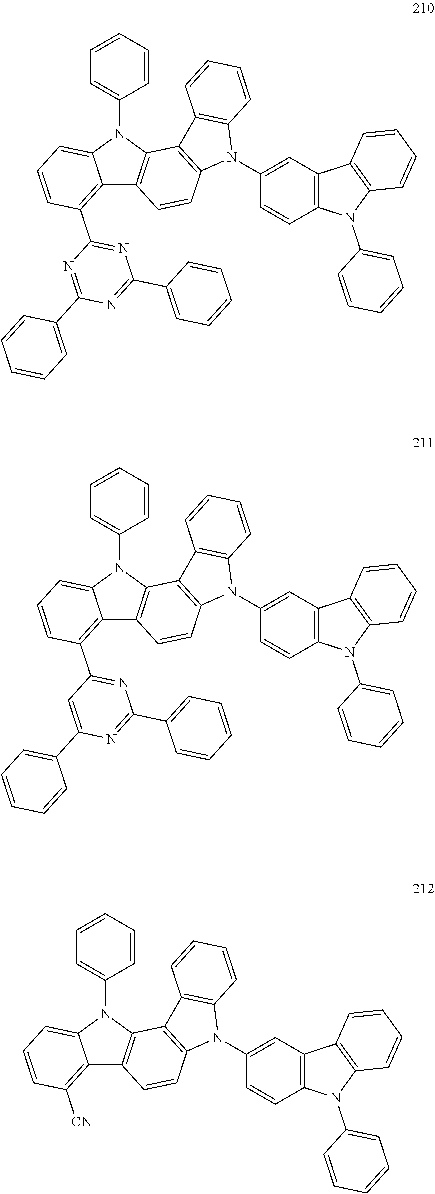 [SMILES: c1ccc(-c2nc(-c3ccccc3)nc(-c3cccc4c3c3ccc5c(c6ccccc6n5-c5ccc6c(c5)c5ccccc5n6-c5ccccc5)c3n4-c3ccccc3)n2)cc1 ; c1ccc(-c2cc(-c3cccc4c3c3ccc5c(c6ccccc6n5-c5ccc6c(c5)c5ccccc5n6-c5ccccc5)c3n4-c3ccccc3)nc(-c3ccccc3)n2)cc1 ; N#Cc1cccc2c1c1ccc3c(c4ccccc4n3-c3ccc4c(c3)c3ccccc3n4-c3ccccc3)c1n2-c1ccccc1] 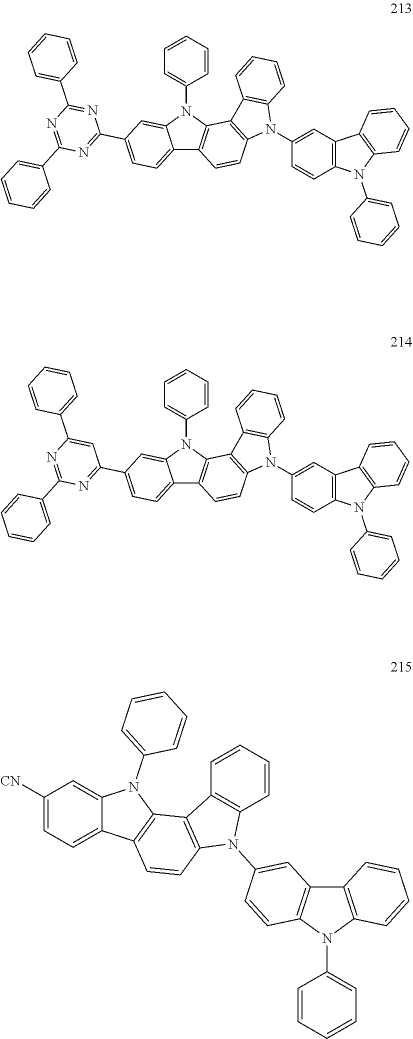 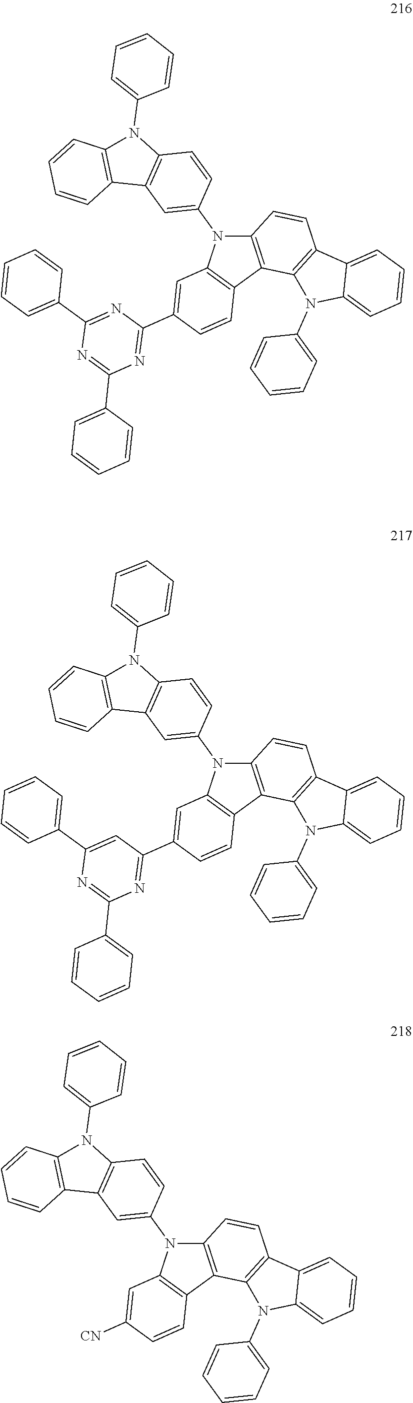 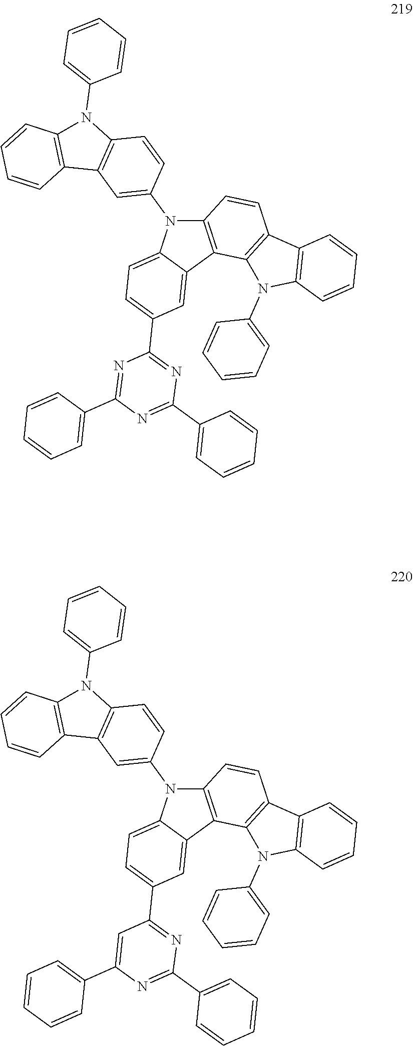 [SMILES: c1ccc(-c2nc(-c3ccccc3)nc(-c3ccc4c(c3)c3c(ccc5c6ccccc6n(-c6ccccc6)c53)n4-c3ccc4c(c3)c3ccccc3n4-c3ccccc3)n2)cc1 ; c1ccc(-c2cc(-c3ccc4c(c3)c3c(ccc5c6ccccc6n(-c6ccccc6)c53)n4-c3ccc4c(c3)c3ccccc3n4-c3ccccc3)nc(-c3ccccc3)n2)cc1] 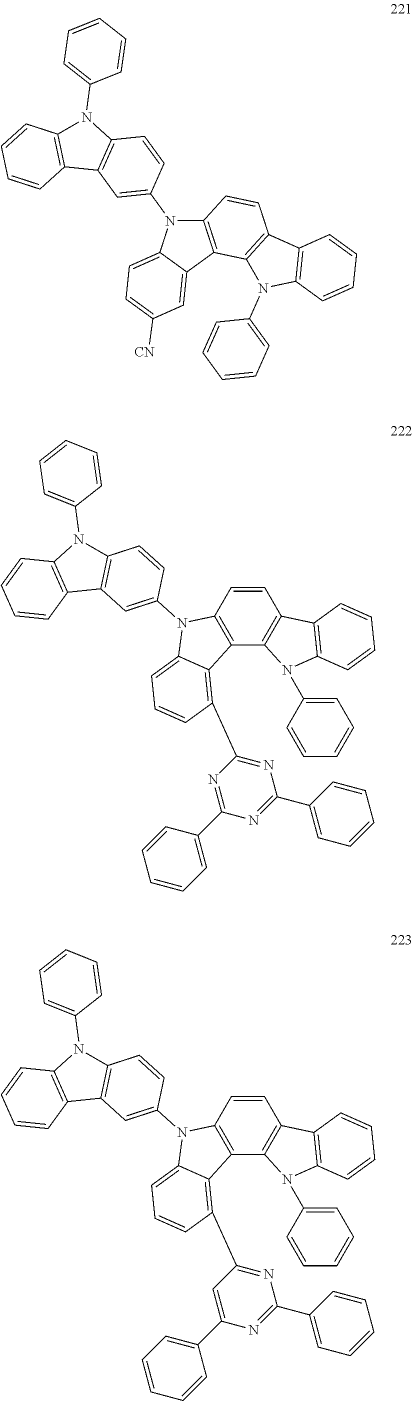 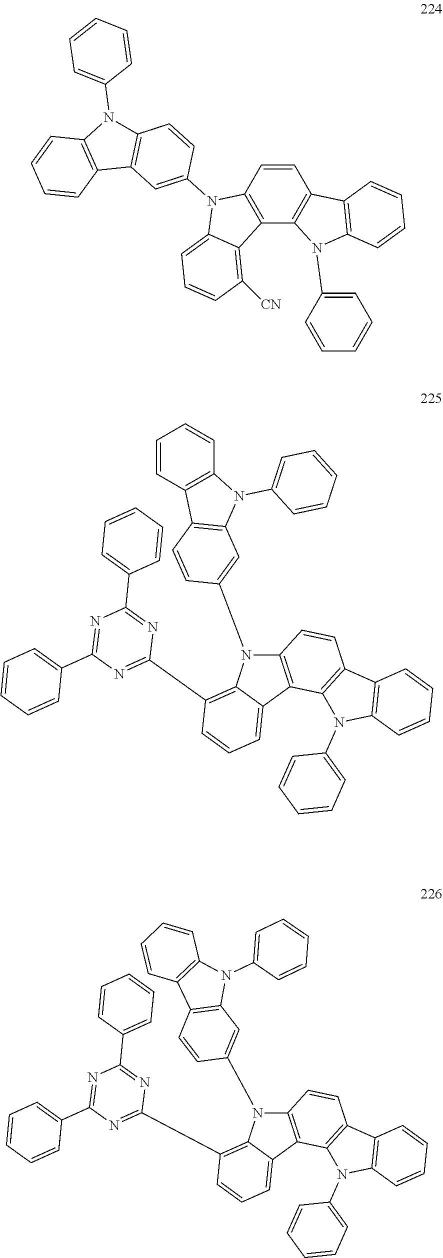 [SMILES: N#Cc1cccc2c1c1c(ccc3c4ccccc4n(-c4ccccc4)c31)n2-c1ccc2c(c1)c1ccccc1n2-c1ccccc1 ; c1ccc(-c2nc(-c3ccccc3)nc(-c3cccc4c5c(ccc6c7ccccc7n(-c7ccccc7)c65)n(-c5ccc6c7ccccc7n(-c7ccccc7)c6c5)c34)n2)cc1 ; c1ccc(-c2nc(-c3ccccc3)nc(-c3cccc4c5c(ccc6c7ccccc7n(-c7ccccc7)c65)n(-c5ccc6c7ccccc7n(-c7ccccc7)c6c5)c34)n2)cc1] 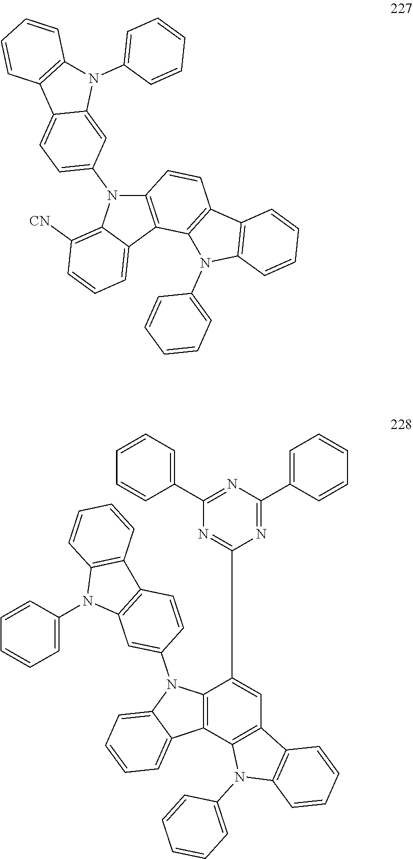 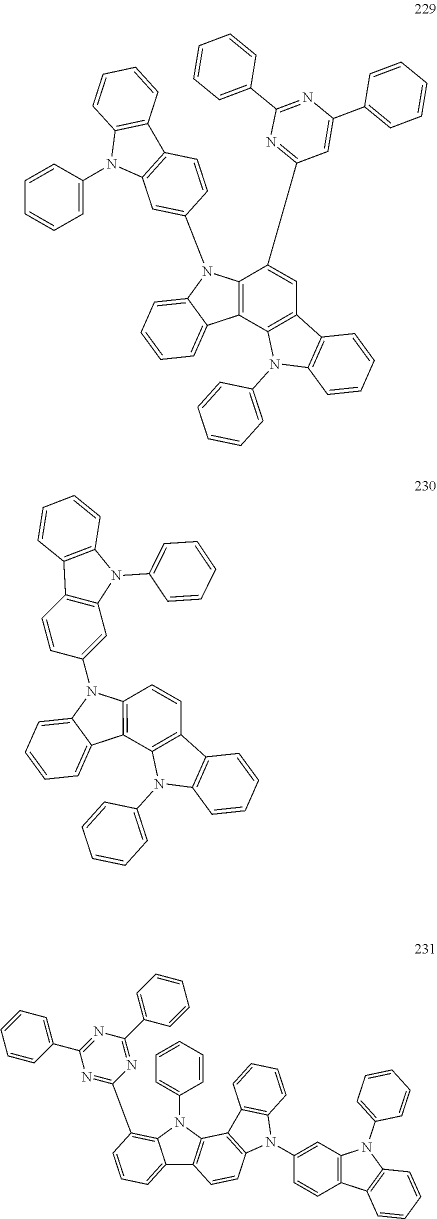 [SMILES: c1ccc(-c2cc(-c3cc4c5ccccc5n(-c5ccccc5)c4c4c5ccccc5n(-c5ccc6c7ccccc7n(-c7ccccc7)c6c5)c34)nc(-c3ccccc3)n2)cc1 ; c1ccc(-n2c3ccccc3c3ccc(-n4c5ccccc5c5c4ccc4c6ccccc6n(-c6ccccc6)c45)cc32)cc1 ; c1ccc(-c2nc(-c3ccccc3)nc(-c3cccc4c5ccc6c(c7ccccc7n6-c6ccc7c8ccccc8n(-c8ccccc8)c7c6)c5n(-c5ccccc5)c34)n2)cc1] 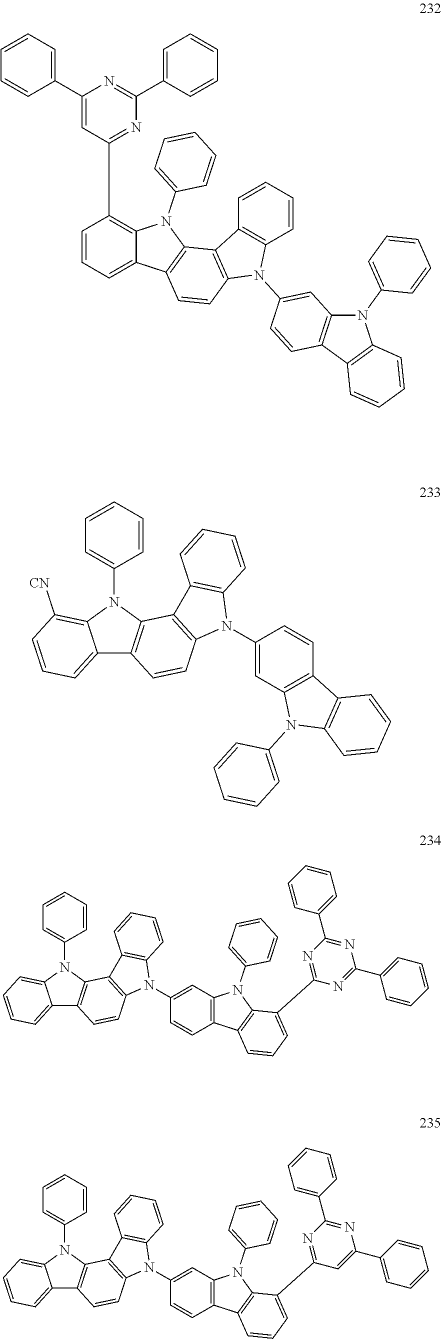 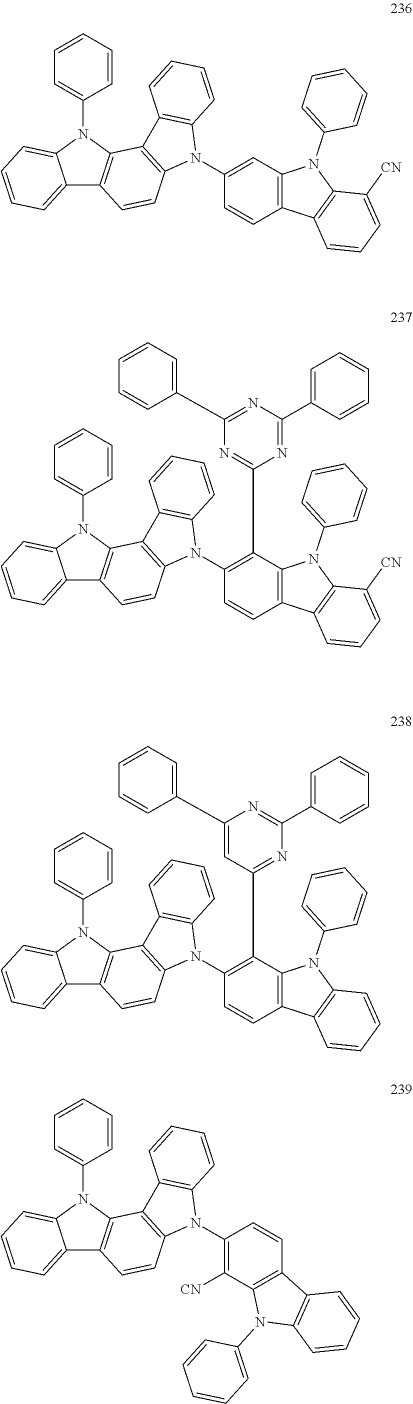 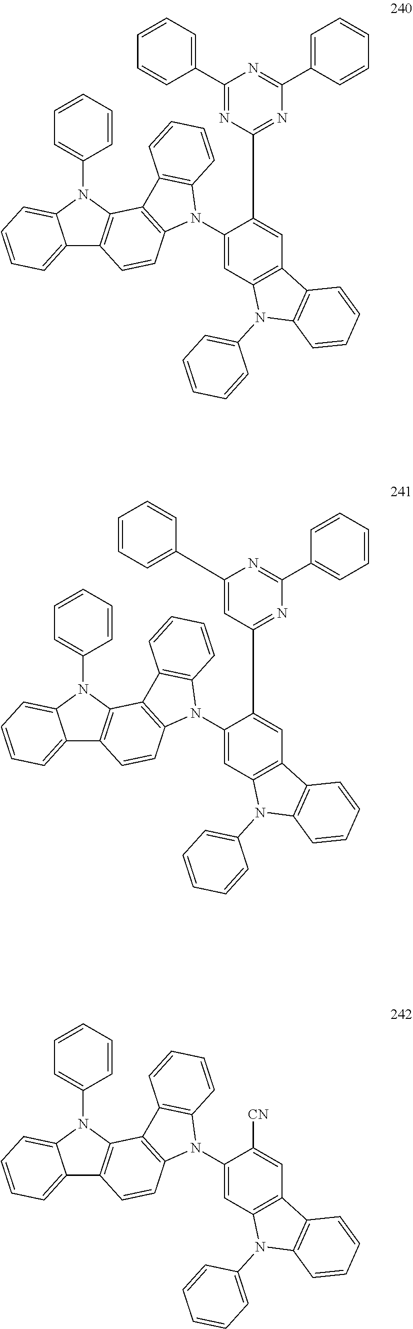 [SMILES: c1ccc(-c2nc(-c3ccccc3)nc(-c3cc4c5ccccc5n(-c5ccccc5)c4cc3-n3c4ccccc4c4c3ccc3c5ccccc5n(-c5ccccc5)c34)n2)cc1 ; c1ccc(-c2cc(-c3cc4c5ccccc5n(-c5ccccc5)c4cc3-n3c4ccccc4c4c3ccc3c5ccccc5n(-c5ccccc5)c34)nc(-c3ccccc3)n2)cc1 ; N#Cc1cc2c3ccccc3n(-c3ccccc3)c2cc1-n1c2ccccc2c2c1ccc1c3ccccc3n(-c3ccccc3)c12] 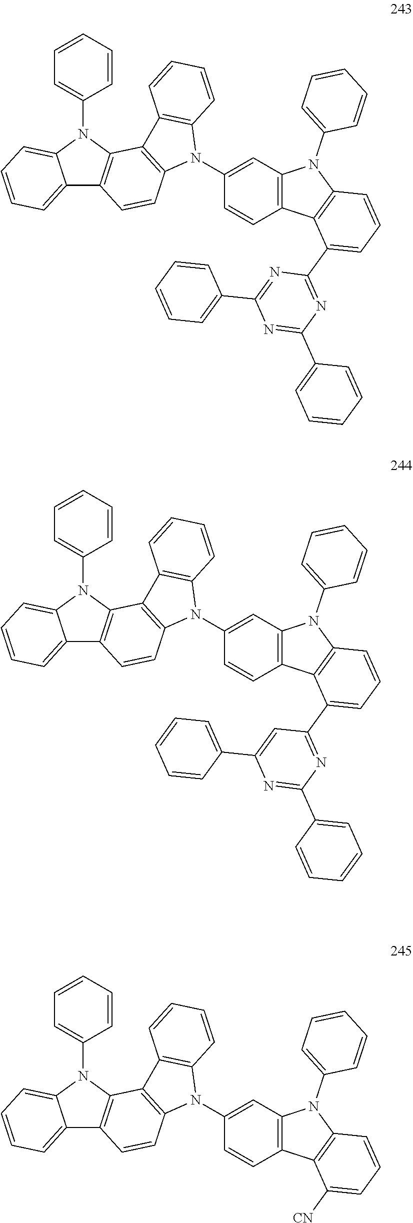 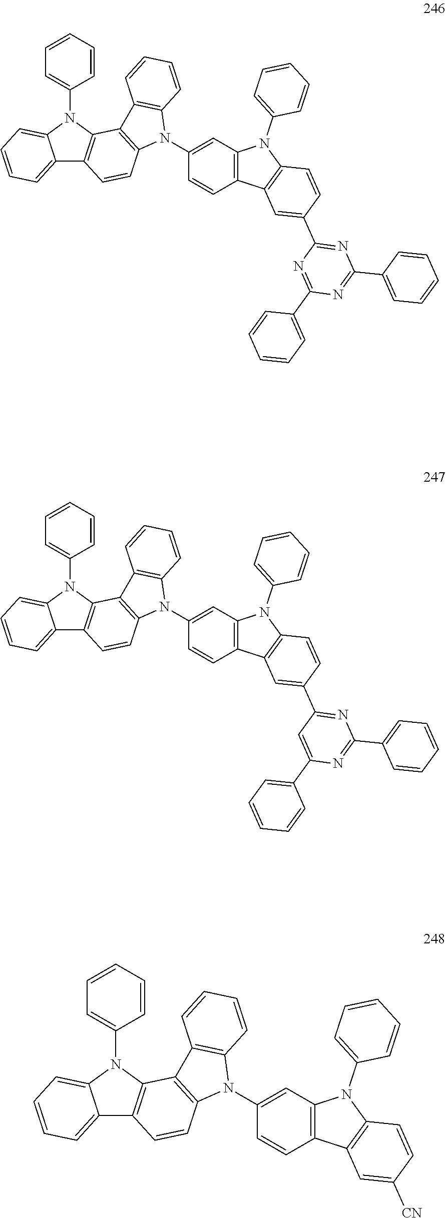 [SMILES: c1ccc(-c2nc(-c3ccccc3)nc(-c3ccc4c(c3)c3ccc(-n5c6ccccc6c6c5ccc5c7ccccc7n(-c7ccccc7)c56)cc3n4-c3ccccc3)n2)cc1 ; c1ccc(-c2cc(-c3ccc4c(c3)c3ccc(-n5c6ccccc6c6c5ccc5c7ccccc7n(-c7ccccc7)c56)cc3n4-c3ccccc3)nc(-c3ccccc3)n2)cc1 ; N#Cc1ccc2c(c1)c1ccc(-n3c4ccccc4c4c3ccc3c5ccccc5n(-c5ccccc5)c34)cc1n2-c1ccccc1] 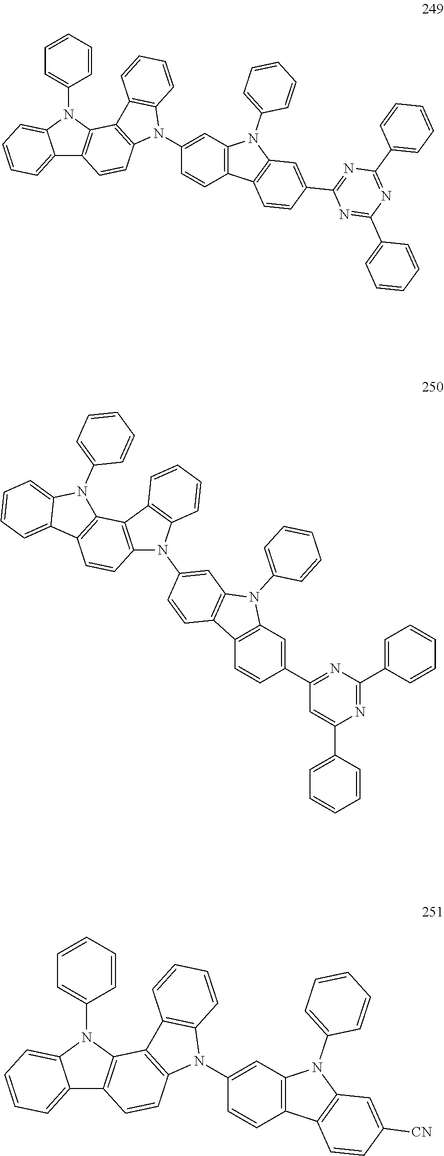 [SMILES: c1ccc(-c2nc(-c3ccccc3)nc(-c3ccc4c5ccc(-n6c7ccccc7c7c6ccc6c8ccccc8n(-c8ccccc8)c67)cc5n(-c5ccccc5)c4c3)n2)cc1 ; c1ccc(-c2cc(-c3ccc4c5ccc(-n6c7ccccc7c7c6ccc6c8ccccc8n(-c8ccccc8)c67)cc5n(-c5ccccc5)c4c3)nc(-c3ccccc3)n2)cc1 ; N#Cc1ccc2c3ccc(-n4c5ccccc5c5c4ccc4c6ccccc6n(-c6ccccc6)c45)cc3n(-c3ccccc3)c2c1] 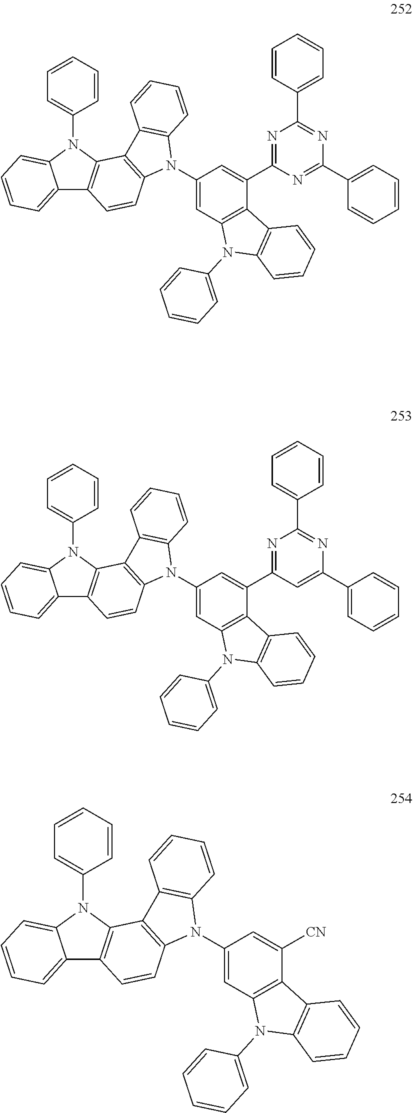 [SMILES: c1ccc(-c2nc(-c3ccccc3)nc(-c3cc(-n4c5ccccc5c5c4ccc4c6ccccc6n(-c6ccccc6)c45)cc4c3c3ccccc3n4-c3ccccc3)n2)cc1 ; c1ccc(-c2cc(-c3cc(-n4c5ccccc5c5c4ccc4c6ccccc6n(-c6ccccc6)c45)cc4c3c3ccccc3n4-c3ccccc3)nc(-c3ccccc3)n2)cc1 ; N#Cc1cc(-n2c3ccccc3c3c2ccc2c4ccccc4n(-c4ccccc4)c23)cc2c1c1ccccc1n2-c1ccccc1] 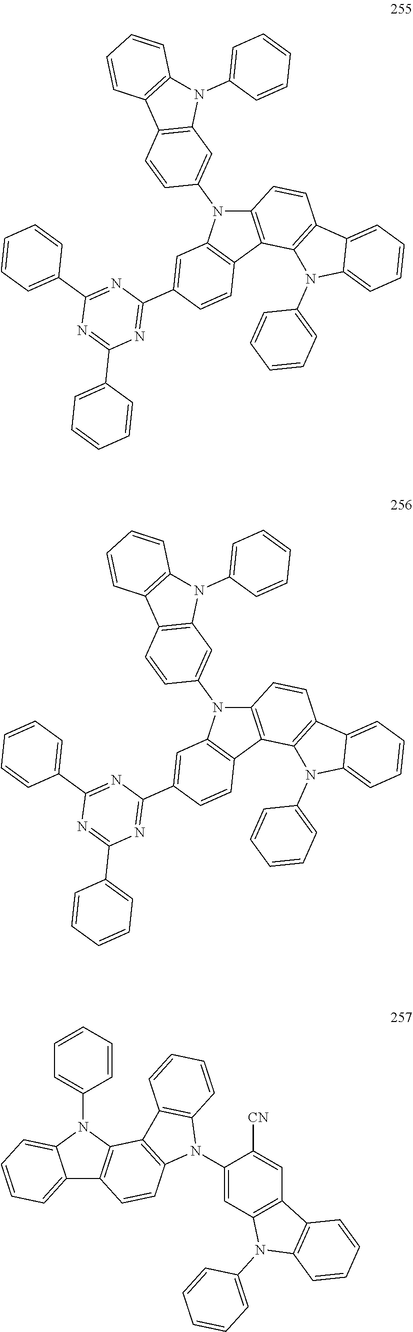 [SMILES: c1ccc(-c2nc(-c3ccccc3)nc(-c3ccc4c5c(ccc6c7ccccc7n(-c7ccccc7)c65)n(-c5ccc6c7ccccc7n(-c7ccccc7)c6c5)c4c3)n2)cc1 ; c1ccc(-c2nc(-c3ccccc3)nc(-c3ccc4c5c(ccc6c7ccccc7n(-c7ccccc7)c65)n(-c5ccc6c7ccccc7n(-c7ccccc7)c6c5)c4c3)n2)cc1 ; N#Cc1cc2c3ccccc3n(-c3ccccc3)c2cc1-n1c2ccccc2c2c1ccc1c3ccccc3n(-c3ccccc3)c12] 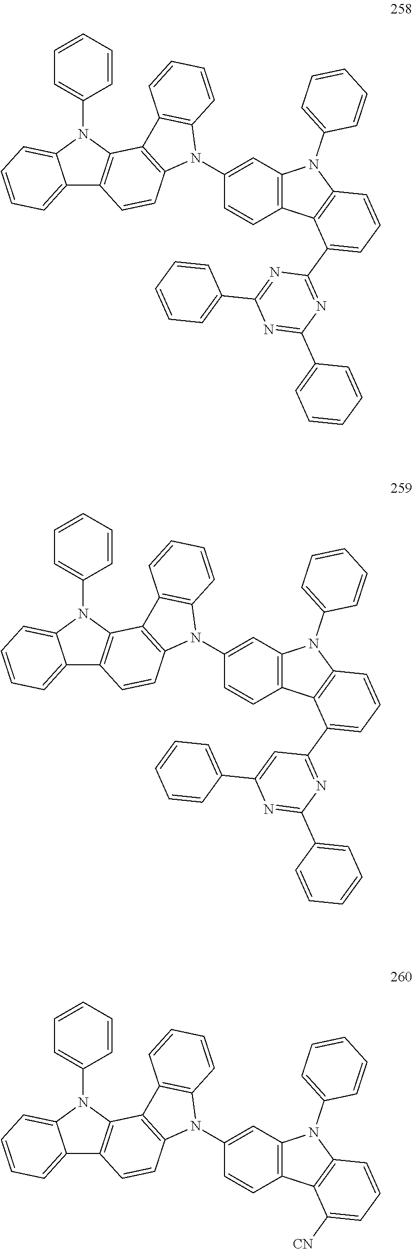 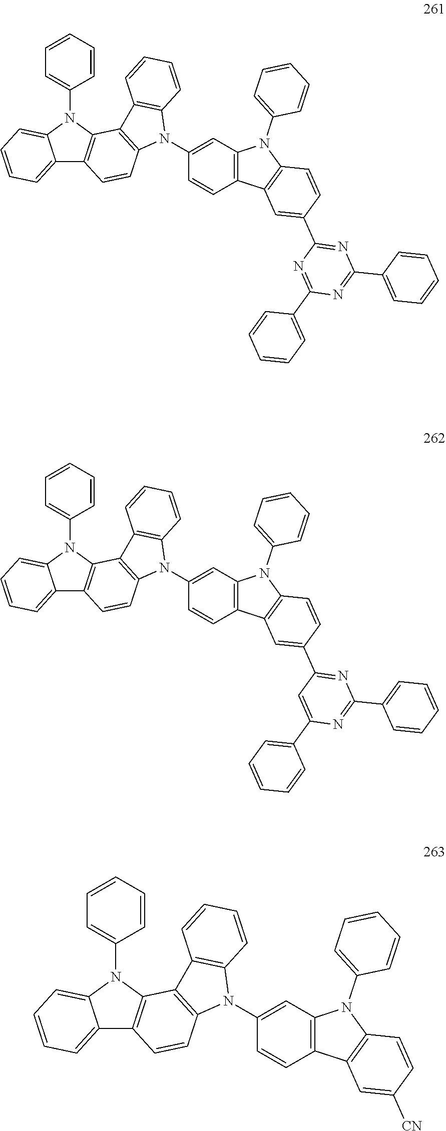 [SMILES: c1ccc(-c2nc(-c3ccccc3)nc(-c3ccc4c(c3)c3ccc(-n5c6ccccc6c6c5ccc5c7ccccc7n(-c7ccccc7)c56)cc3n4-c3ccccc3)n2)cc1 ; c1ccc(-c2cc(-c3ccc4c(c3)c3ccc(-n5c6ccccc6c6c5ccc5c7ccccc7n(-c7ccccc7)c56)cc3n4-c3ccccc3)nc(-c3ccccc3)n2)cc1 ; N#Cc1ccc2c(c1)c1ccc(-n3c4ccccc4c4c3ccc3c5ccccc5n(-c5ccccc5)c34)cc1n2-c1ccccc1] 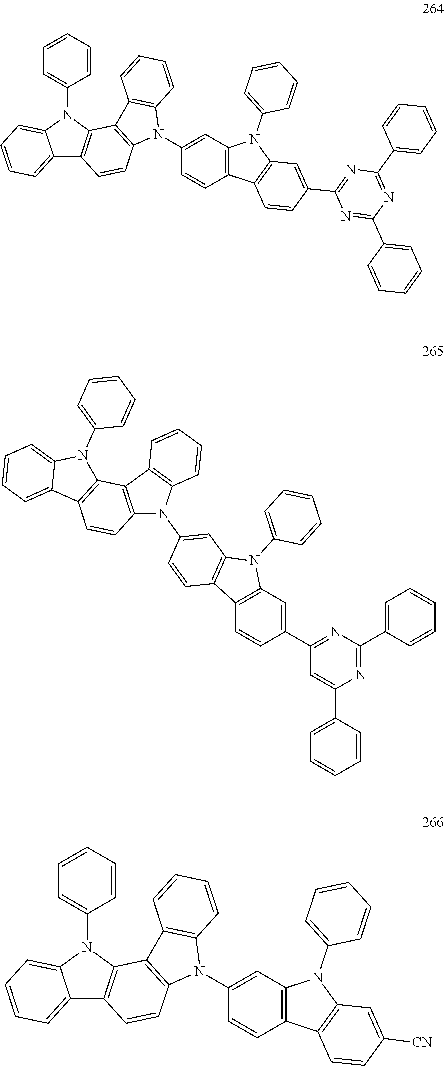 [SMILES: c1ccc(-c2nc(-c3ccccc3)nc(-c3ccc4c5ccc(-n6c7ccccc7c7c6ccc6c8ccccc8n(-c8ccccc8)c67)cc5n(-c5ccccc5)c4c3)n2)cc1 ; c1ccc(-c2cc(-c3ccc4c5ccc(-n6c7ccccc7c7c6ccc6c8ccccc8n(-c8ccccc8)c67)cc5n(-c5ccccc5)c4c3)nc(-c3ccccc3)n2)cc1 ; N#Cc1ccc2c3ccc(-n4c5ccccc5c5c4ccc4c6ccccc6n(-c6ccccc6)c45)cc3n(-c3ccccc3)c2c1] 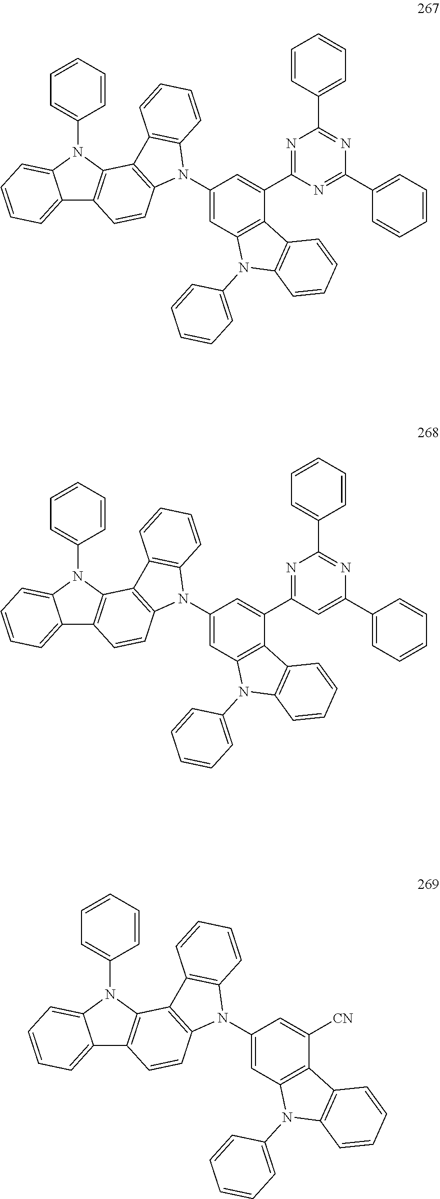 [SMILES: c1ccc(-c2nc(-c3ccccc3)nc(-c3cc(-n4c5ccccc5c5c4ccc4c6ccccc6n(-c6ccccc6)c45)cc4c3c3ccccc3n4-c3ccccc3)n2)cc1 ; c1ccc(-c2cc(-c3cc(-n4c5ccccc5c5c4ccc4c6ccccc6n(-c6ccccc6)c45)cc4c3c3ccccc3n4-c3ccccc3)nc(-c3ccccc3)n2)cc1 ; N#Cc1cc(-n2c3ccccc3c3c2ccc2c4ccccc4n(-c4ccccc4)c23)cc2c1c1ccccc1n2-c1ccccc1] 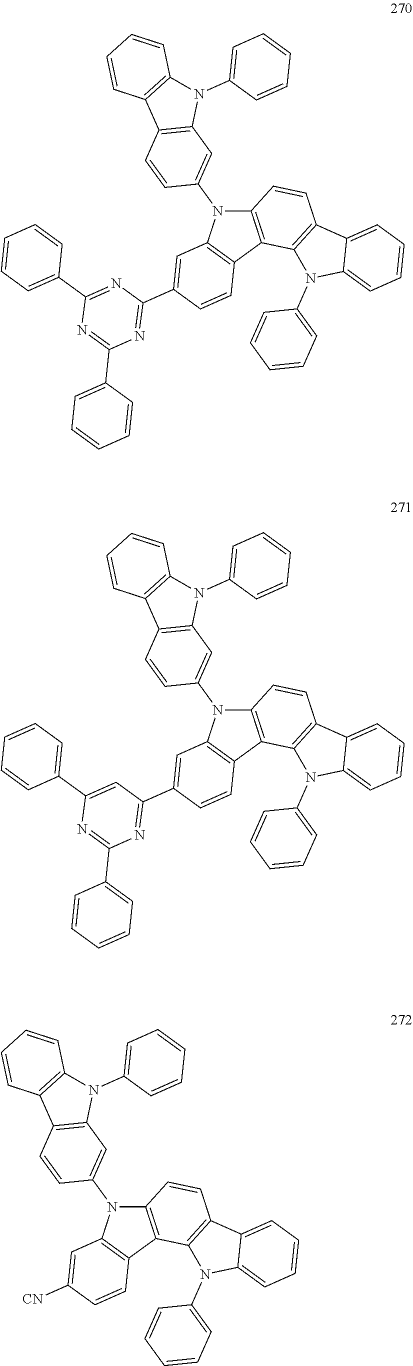 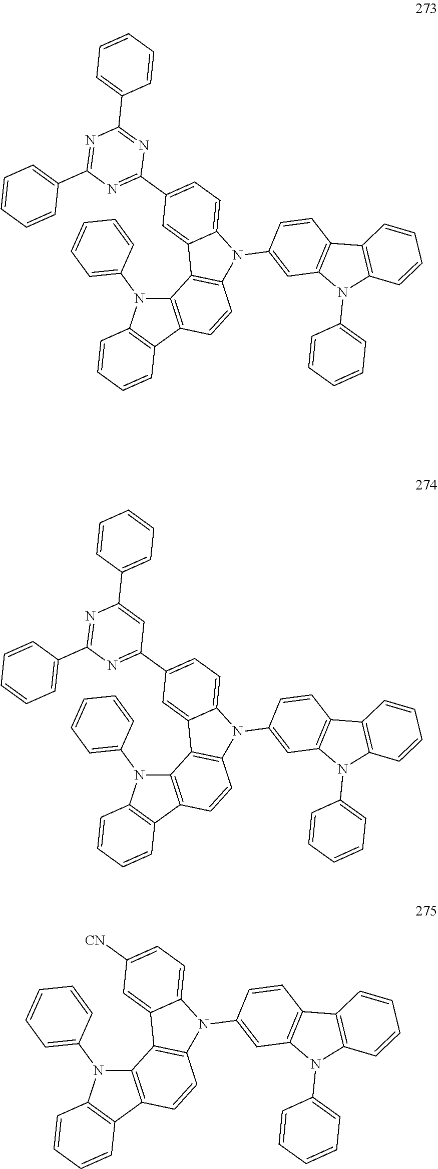 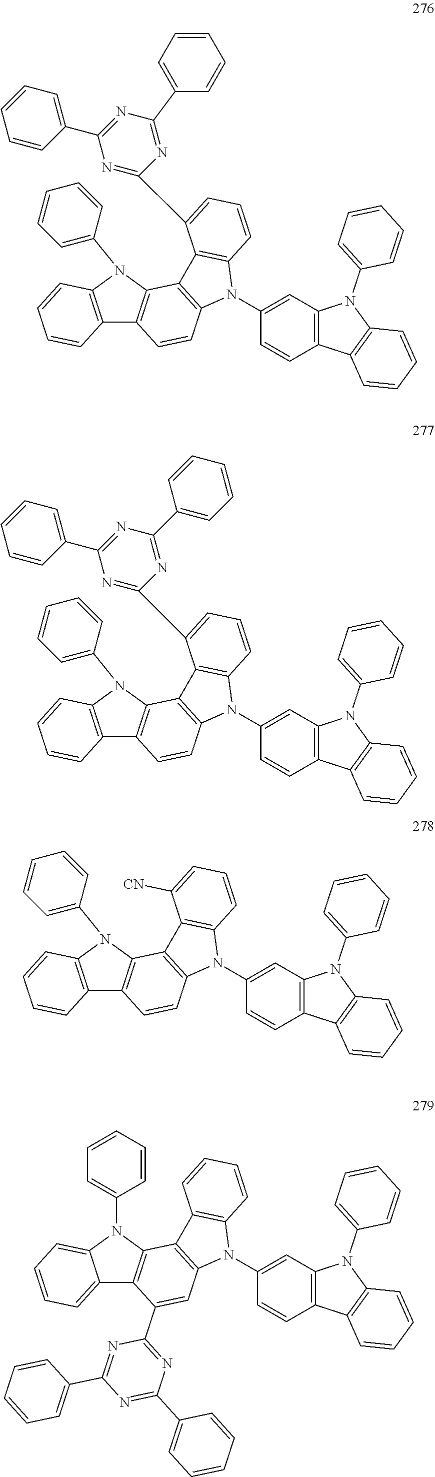 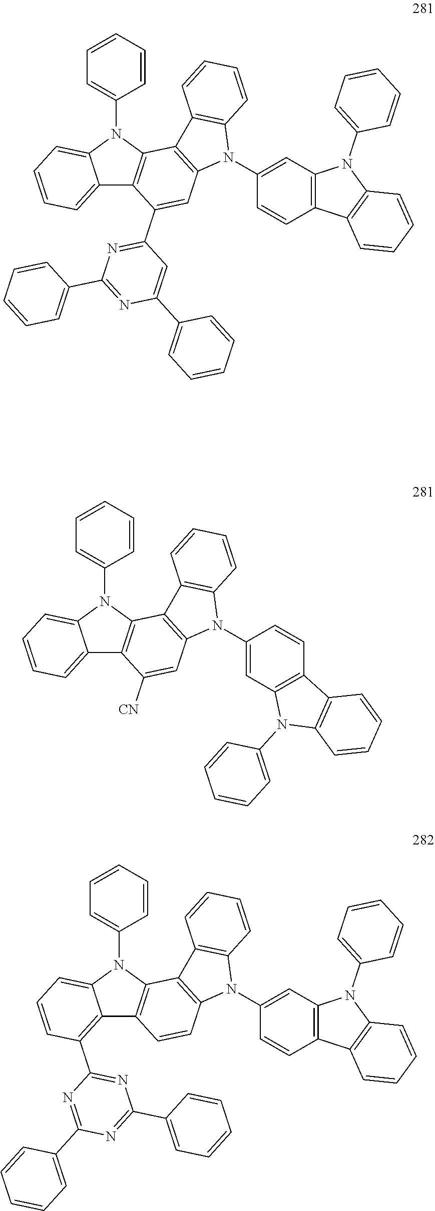 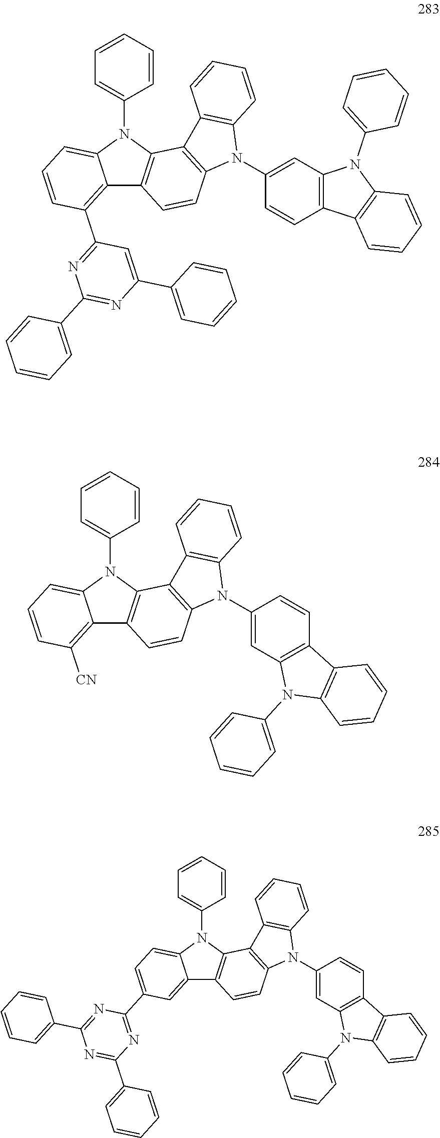 [SMILES: c1ccc(-c2cc(-c3cccc4c3c3ccc5c(c6ccccc6n5-c5ccc6c7ccccc7n(-c7ccccc7)c6c5)c3n4-c3ccccc3)nc(-c3ccccc3)n2)cc1 ; N#Cc1cccc2c1c1ccc3c(c4ccccc4n3-c3ccc4c5ccccc5n(-c5ccccc5)c4c3)c1n2-c1ccccc1 ; c1ccc(-c2nc(-c3ccccc3)nc(-c3ccc4c(c3)c3ccc5c(c6ccccc6n5-c5ccc6c7ccccc7n(-c7ccccc7)c6c5)c3n4-c3ccccc3)n2)cc1] 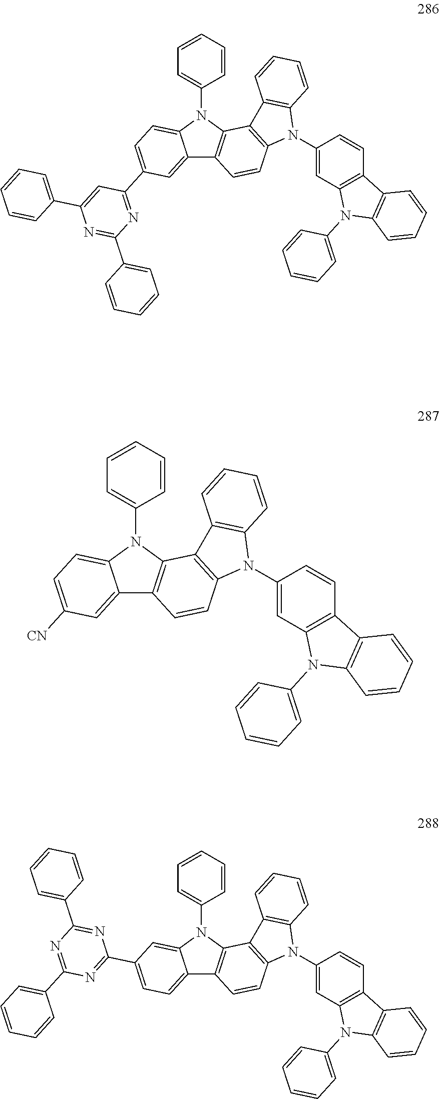 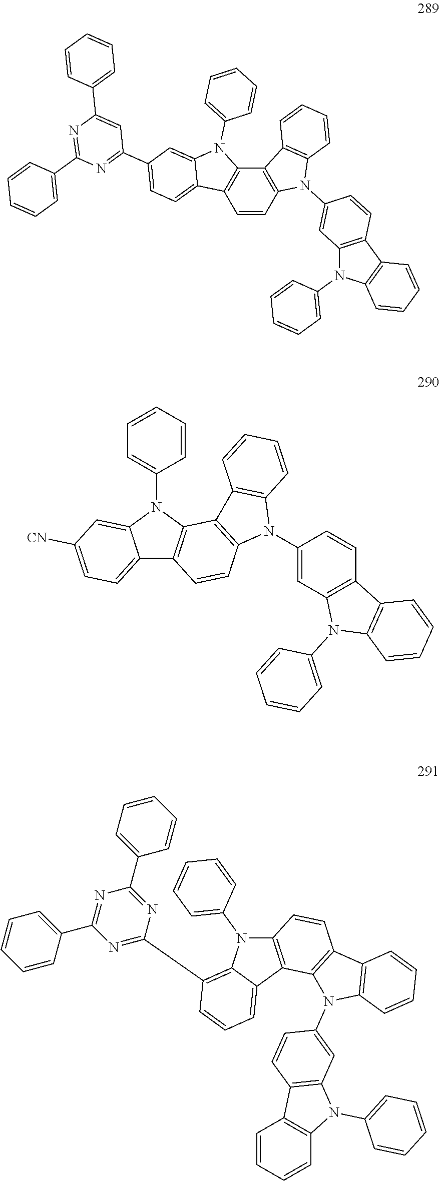 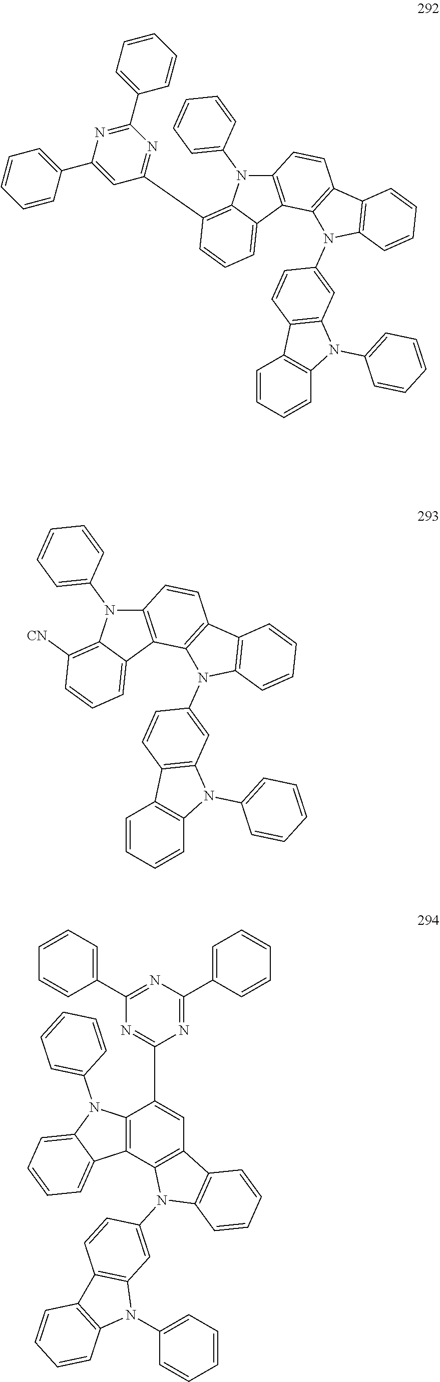 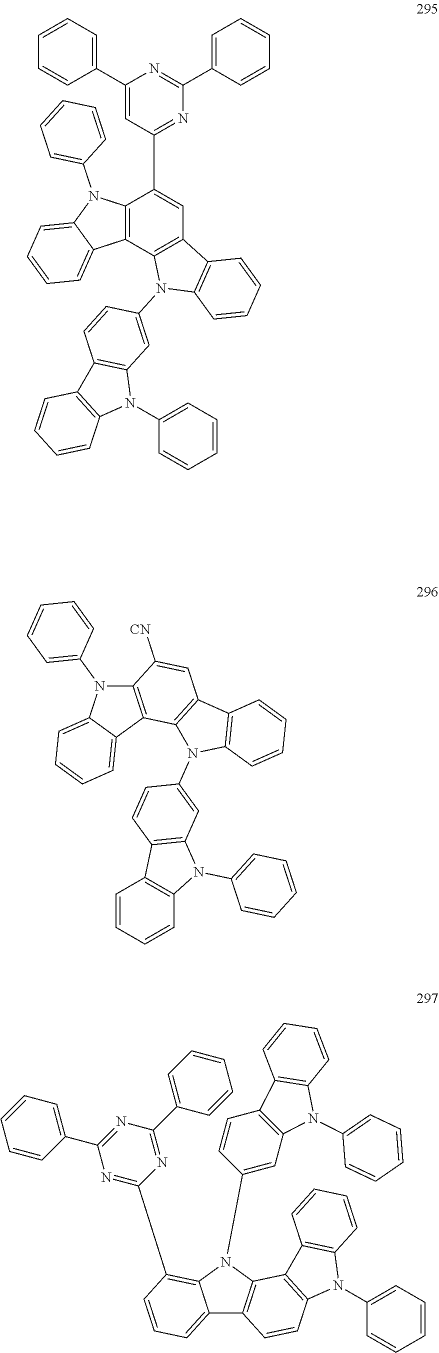 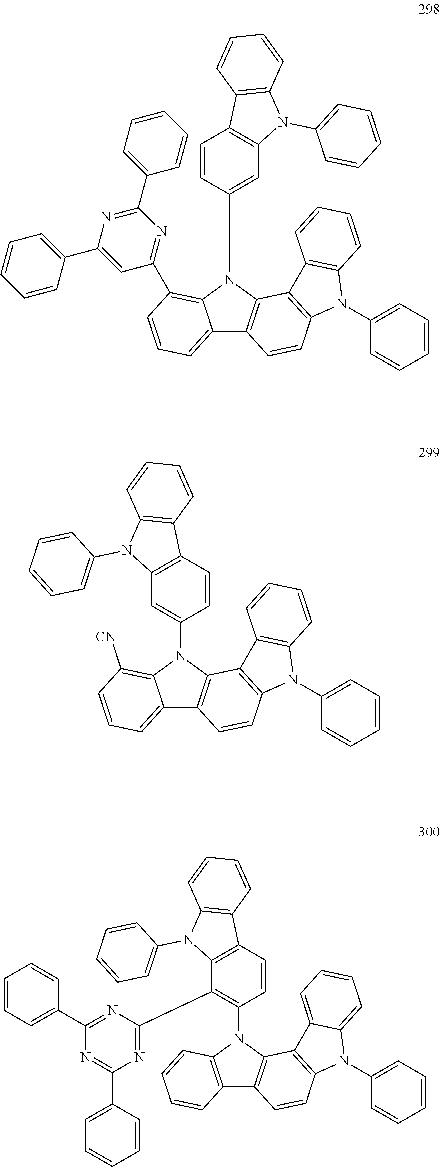 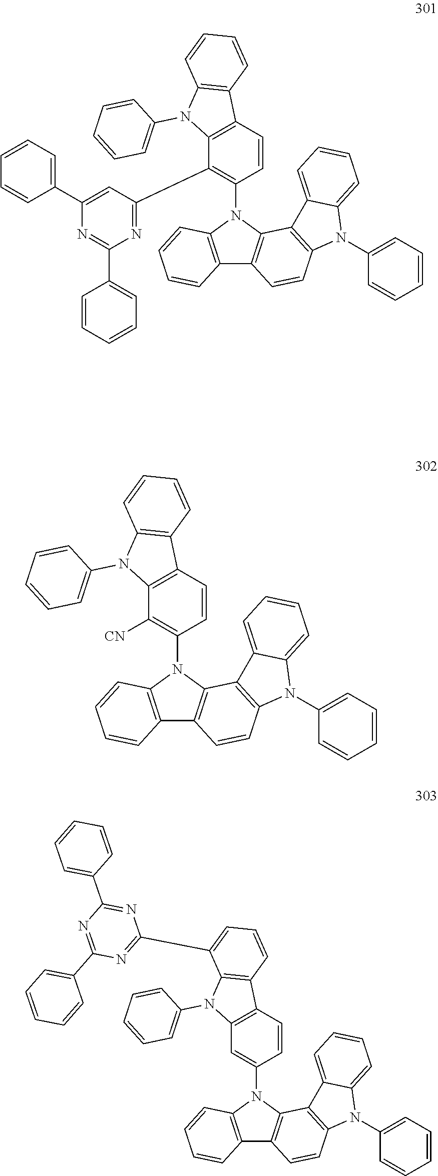 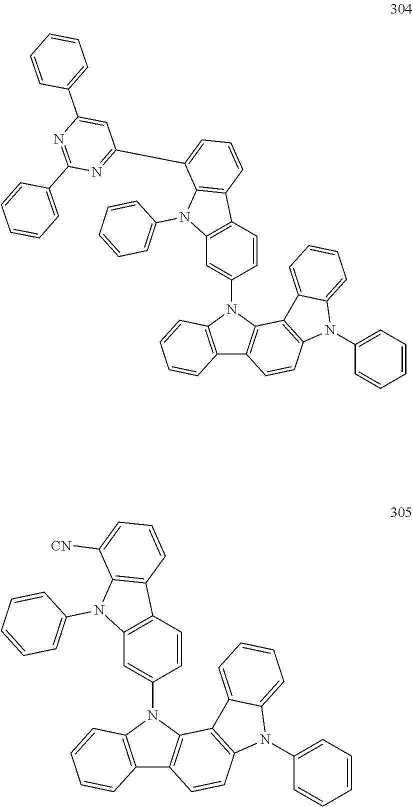 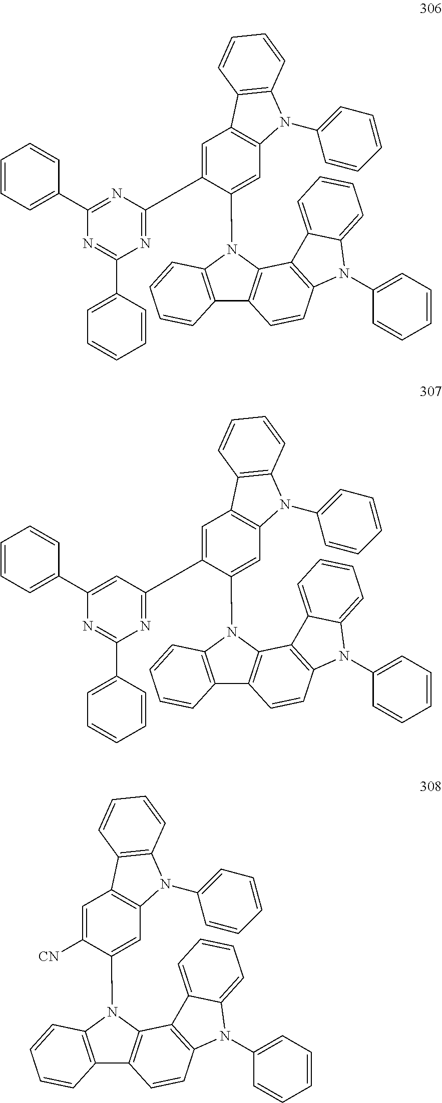 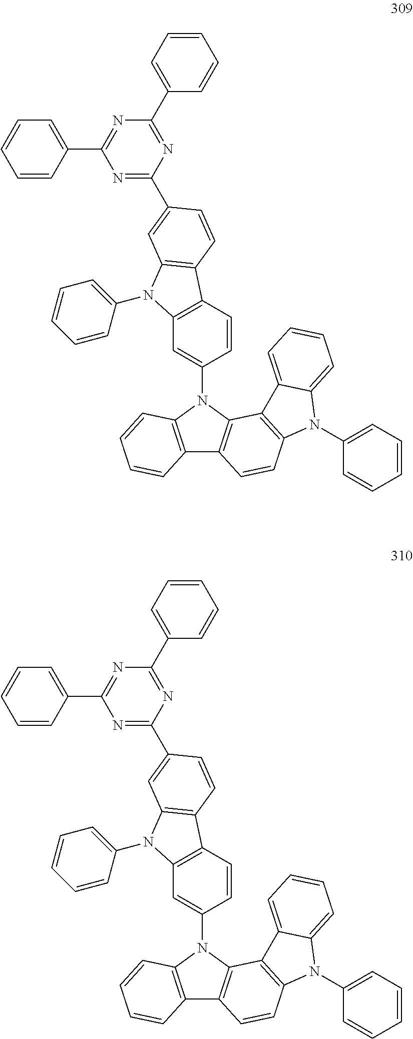 [SMILES: c1ccc(-c2nc(-c3ccccc3)nc(-c3ccc4c5ccc(-n6c7ccccc7c7ccc8c(c9ccccc9n8-c8ccccc8)c76)cc5n(-c5ccccc5)c4c3)n2)cc1 ; c1ccc(-c2nc(-c3ccccc3)nc(-c3ccc4c5ccc(-n6c7ccccc7c7ccc8c(c9ccccc9n8-c8ccccc8)c76)cc5n(-c5ccccc5)c4c3)n2)cc1] 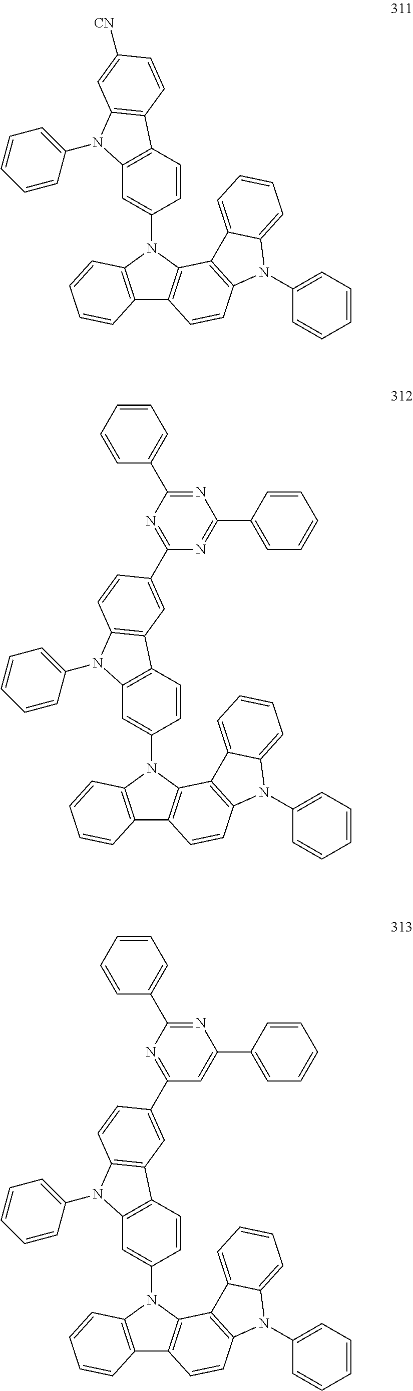 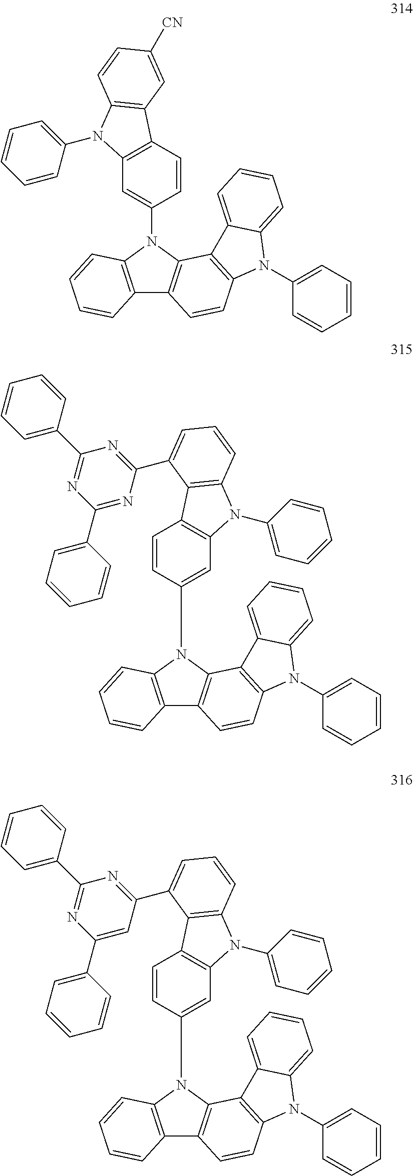 [SMILES: N#Cc1ccc2c(c1)c1ccc(-n3c4ccccc4c4ccc5c(c6ccccc6n5-c5ccccc5)c43)cc1n2-c1ccccc1 ; c1ccc(-c2nc(-c3ccccc3)nc(-c3cccc4c3c3ccc(-n5c6ccccc6c6ccc7c(c8ccccc8n7-c7ccccc7)c65)cc3n4-c3ccccc3)n2)cc1 ; c1ccc(-c2cc(-c3cccc4c3c3ccc(-n5c6ccccc6c6ccc7c(c8ccccc8n7-c7ccccc7)c65)cc3n4-c3ccccc3)nc(-c3ccccc3)n2)cc1] 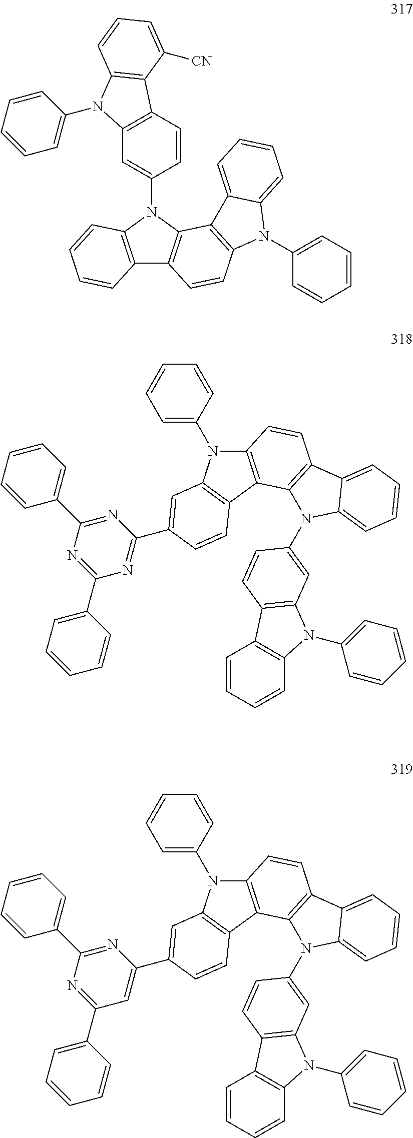 [SMILES: N#Cc1cccc2c1c1ccc(-n3c4ccccc4c4ccc5c(c6ccccc6n5-c5ccccc5)c43)cc1n2-c1ccccc1 ; c1ccc(-c2nc(-c3ccccc3)nc(-c3ccc4c5c(ccc6c7ccccc7n(-c7ccc8c9ccccc9n(-c9ccccc9)c8c7)c65)n(-c5ccccc5)c4c3)n2)cc1 ; c1ccc(-c2cc(-c3ccc4c5c(ccc6c7ccccc7n(-c7ccc8c9ccccc9n(-c9ccccc9)c8c7)c65)n(-c5ccccc5)c4c3)nc(-c3ccccc3)n2)cc1] 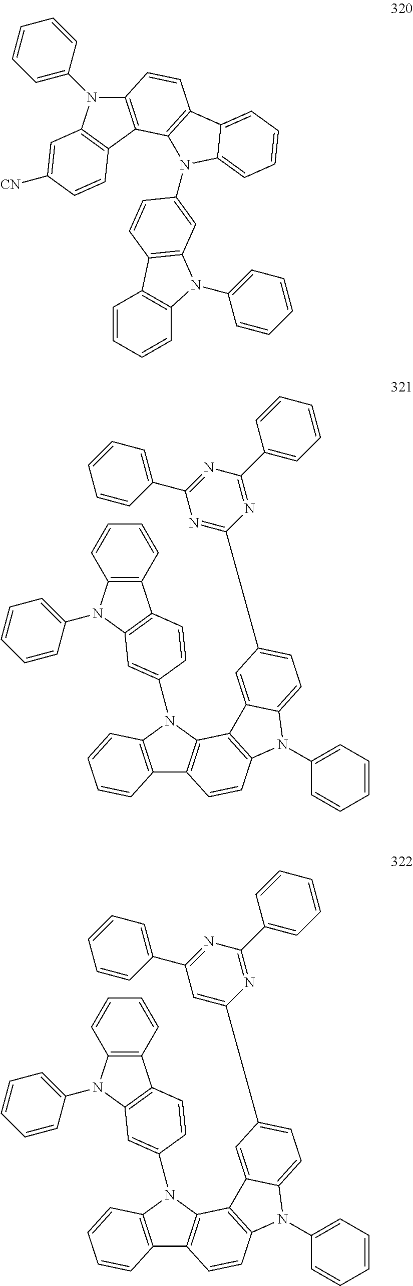 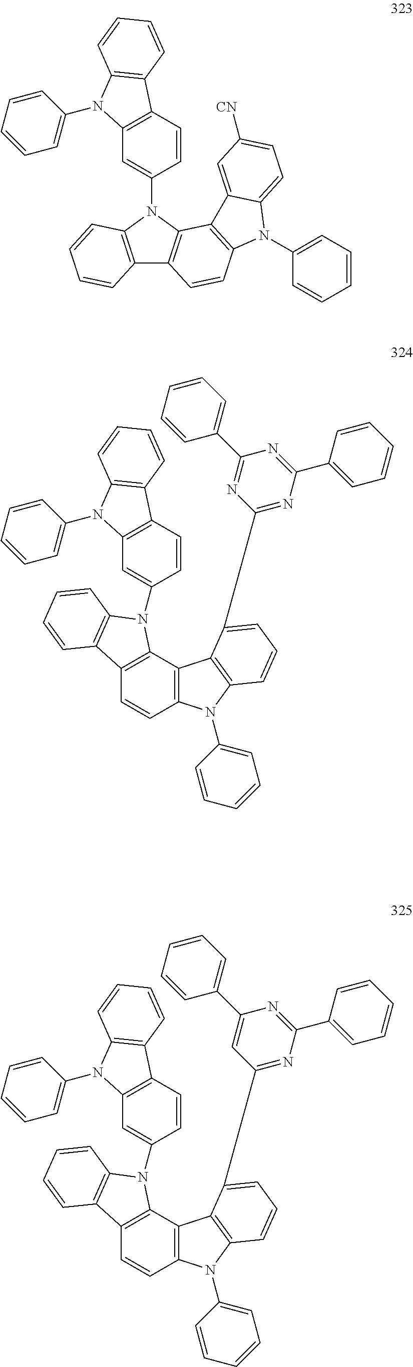 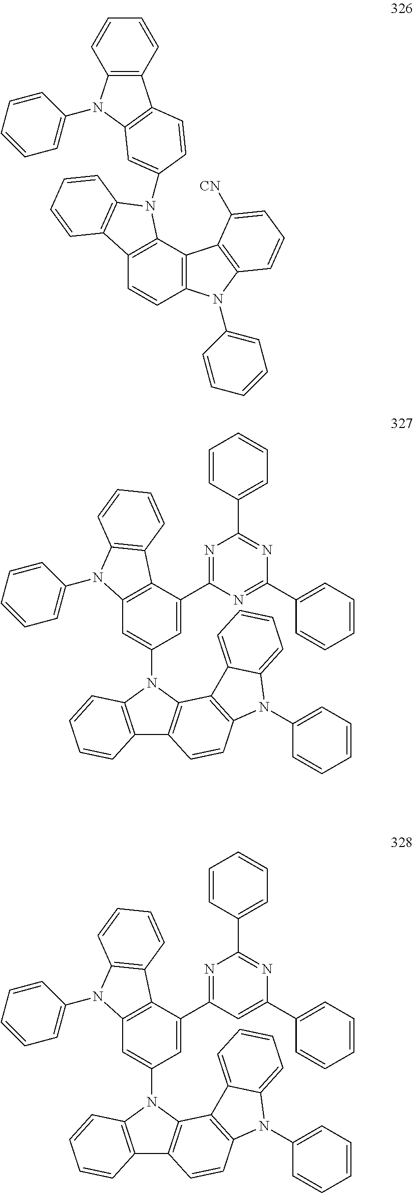 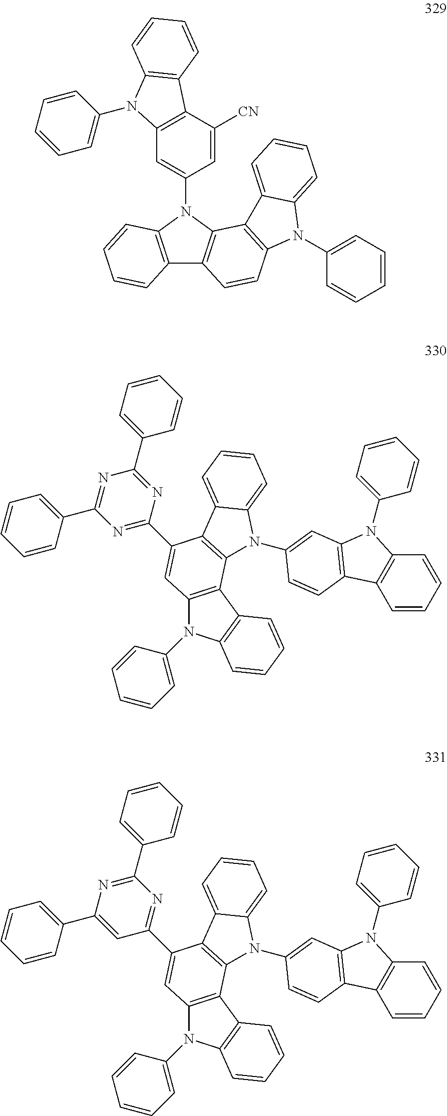 [SMILES: N#Cc1cc(-n2c3ccccc3c3ccc4c(c5ccccc5n4-c4ccccc4)c32)cc2c1c1ccccc1n2-c1ccccc1 ; c1ccc(-c2nc(-c3ccccc3)nc(-c3cc4c(c5ccccc5n4-c4ccccc4)c4c3c3ccccc3n4-c3ccc4c5ccccc5n(-c5ccccc5)c4c3)n2)cc1 ; c1ccc(-c2cc(-c3cc4c(c5ccccc5n4-c4ccccc4)c4c3c3ccccc3n4-c3ccc4c5ccccc5n(-c5ccccc5)c4c3)nc(-c3ccccc3)n2)cc1] 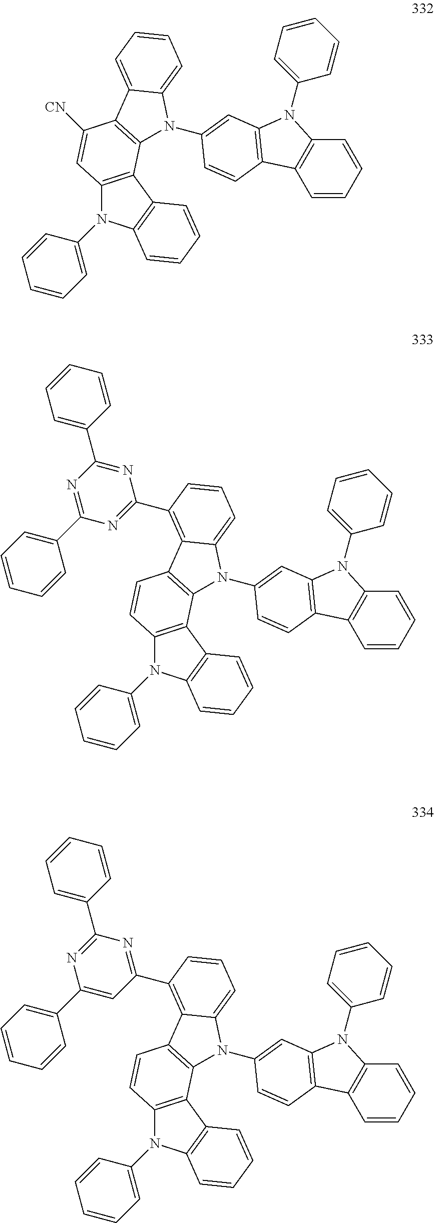 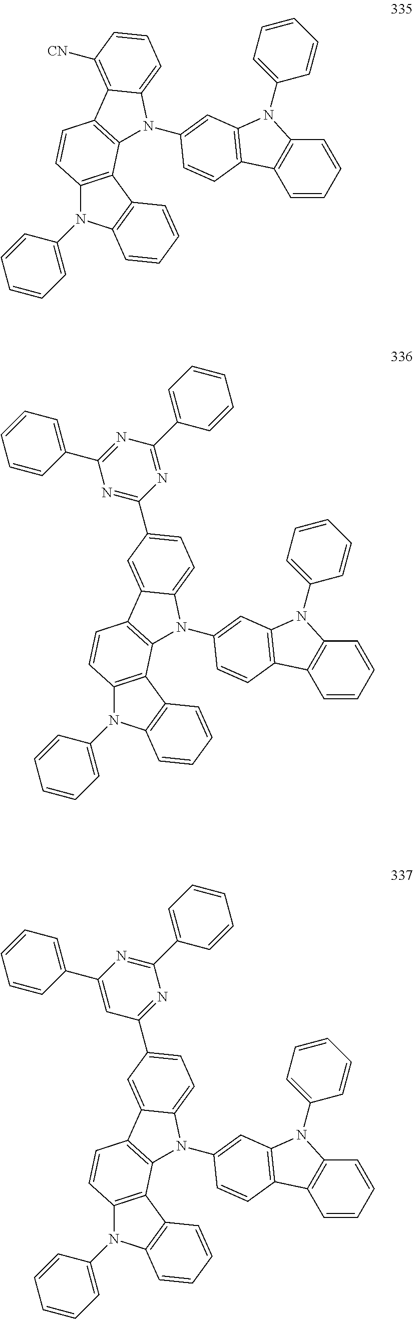 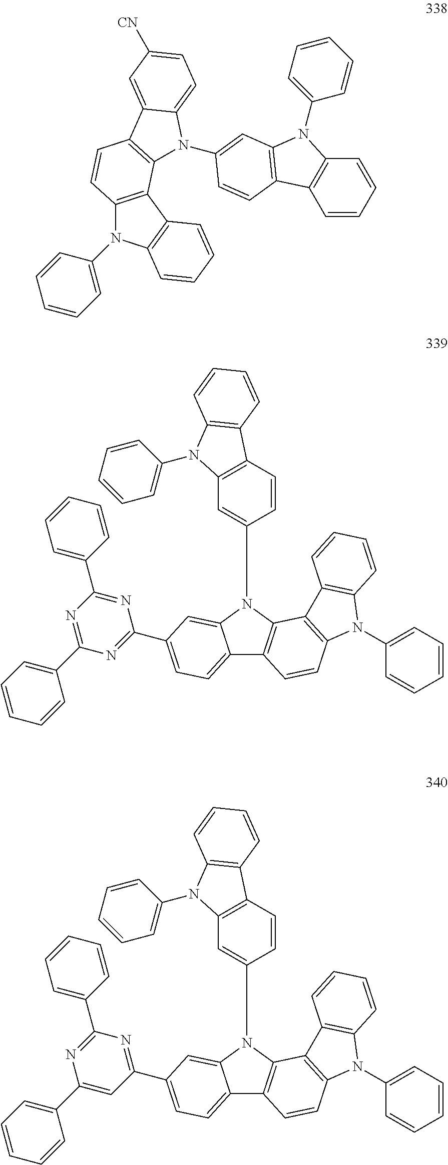 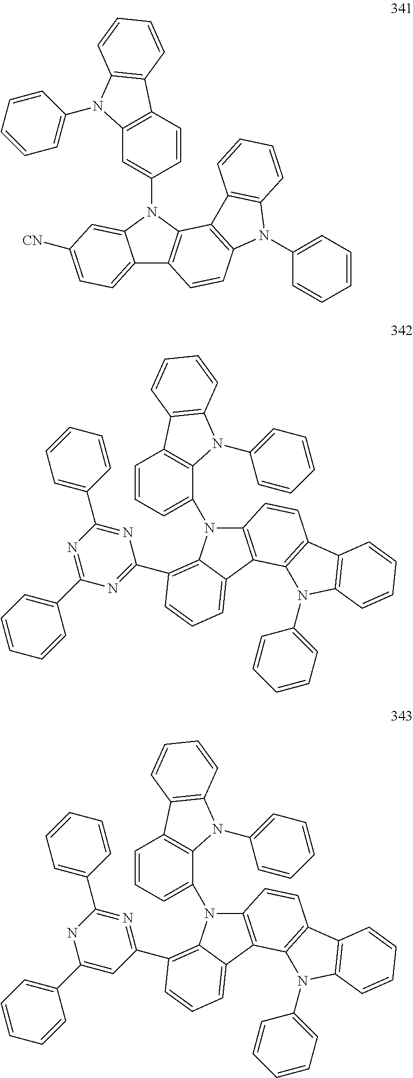 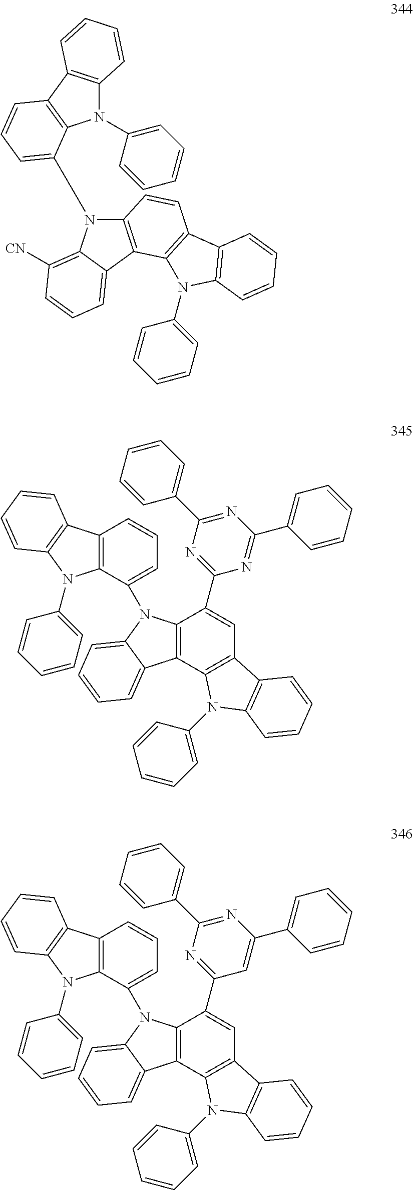 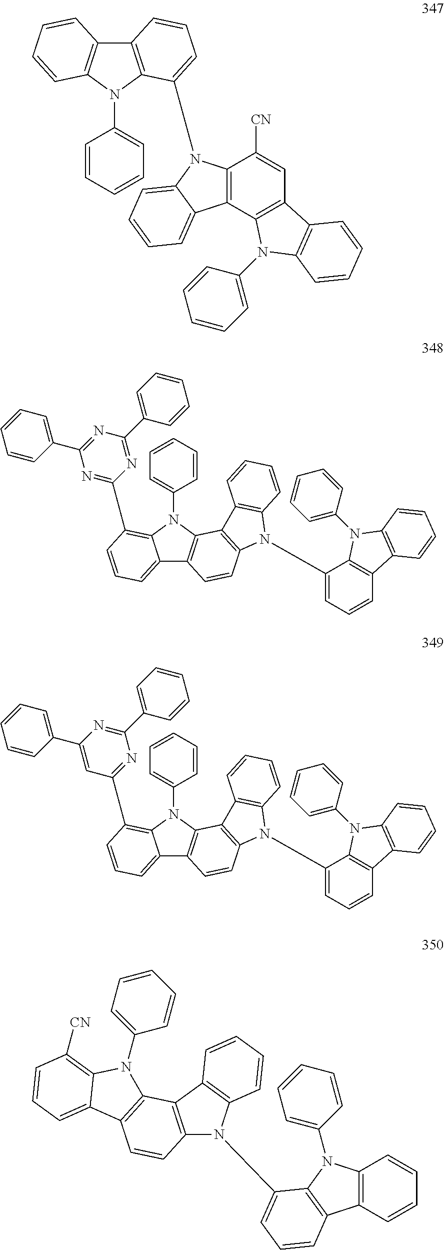 [SMILES: N#Cc1cc2c3ccccc3n(-c3ccccc3)c2c2c3ccccc3n(-c3cccc4c5ccccc5n(-c5ccccc5)c34)c12 ; c1ccc(-c2nc(-c3ccccc3)nc(-c3cccc4c5ccc6c(c7ccccc7n6-c6cccc7c8ccccc8n(-c8ccccc8)c67)c5n(-c5ccccc5)c34)n2)cc1 ; c1ccc(-c2cc(-c3cccc4c5ccc6c(c7ccccc7n6-c6cccc7c8ccccc8n(-c8ccccc8)c67)c5n(-c5ccccc5)c34)nc(-c3ccccc3)n2)cc1 ; N#Cc1cccc2c3ccc4c(c5ccccc5n4-c4cccc5c6ccccc6n(-c6ccccc6)c45)c3n(-c3ccccc3)c12] 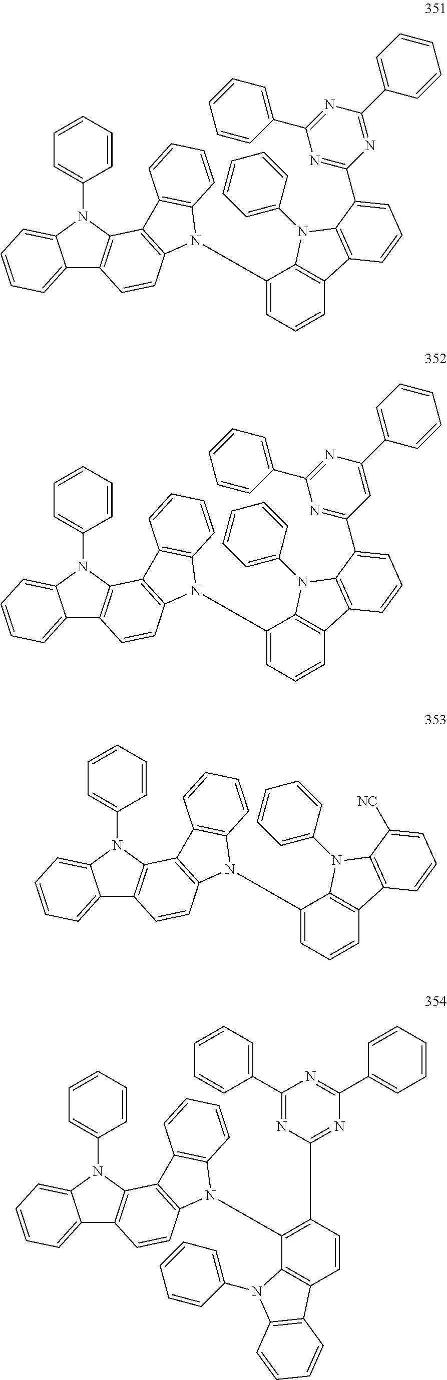 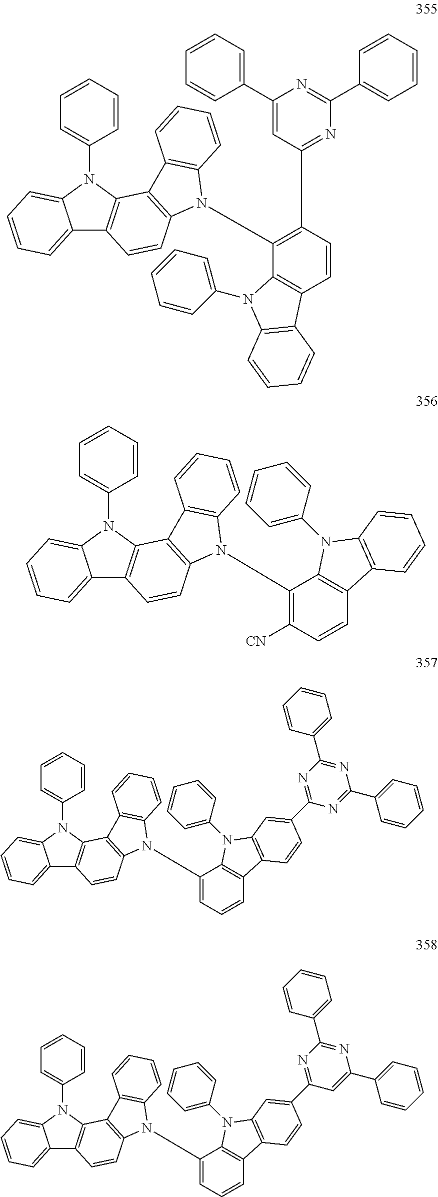 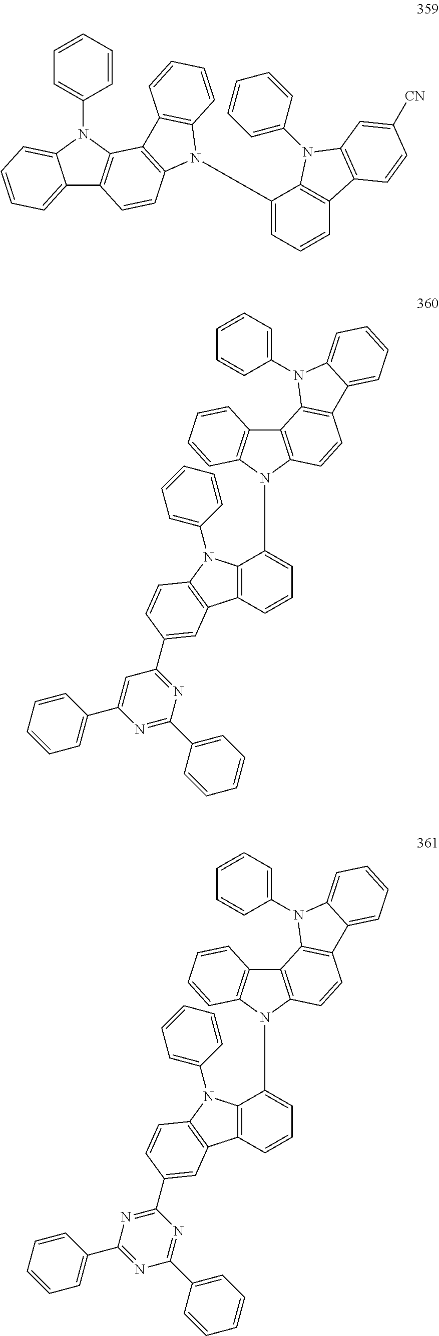 [SMILES: N#Cc1ccc2c3cccc(-n4c5ccccc5c5c4ccc4c6ccccc6n(-c6ccccc6)c45)c3n(-c3ccccc3)c2c1 ; c1ccc(-c2cc(-c3ccc4c(c3)c3cccc(-n5c6ccccc6c6c5ccc5c7ccccc7n(-c7ccccc7)c56)c3n4-c3ccccc3)nc(-c3ccccc3)n2)cc1 ; c1ccc(-c2nc(-c3ccccc3)nc(-c3ccc4c(c3)c3cccc(-n5c6ccccc6c6c5ccc5c7ccccc7n(-c7ccccc7)c56)c3n4-c3ccccc3)n2)cc1] 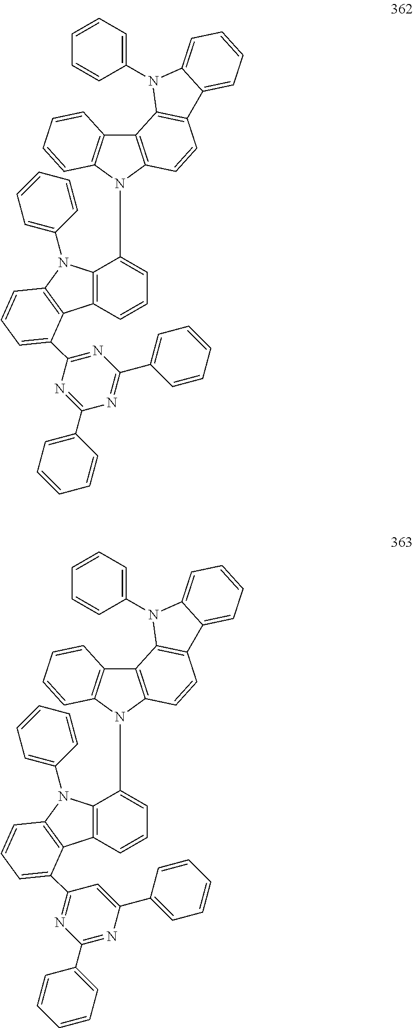 [SMILES: c1ccc(-c2nc(-c3ccccc3)nc(-c3cccc4c3c3cccc(-n5c6ccccc6c6c5ccc5c7ccccc7n(-c7ccccc7)c56)c3n4-c3ccccc3)n2)cc1 ; c1ccc(-c2cc(-c3cccc4c3c3cccc(-n5c6ccccc6c6c5ccc5c7ccccc7n(-c7ccccc7)c56)c3n4-c3ccccc3)nc(-c3ccccc3)n2)cc1] 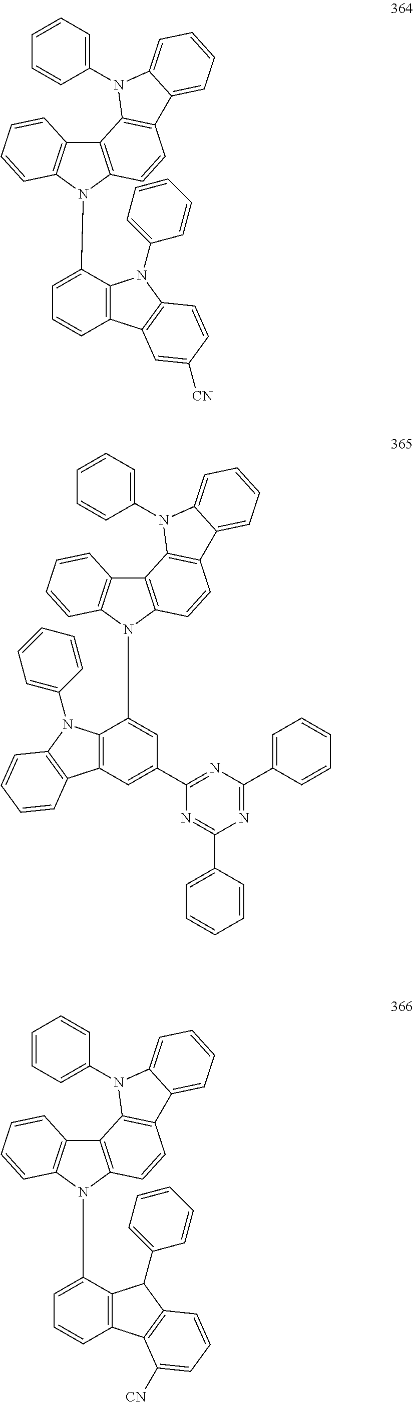 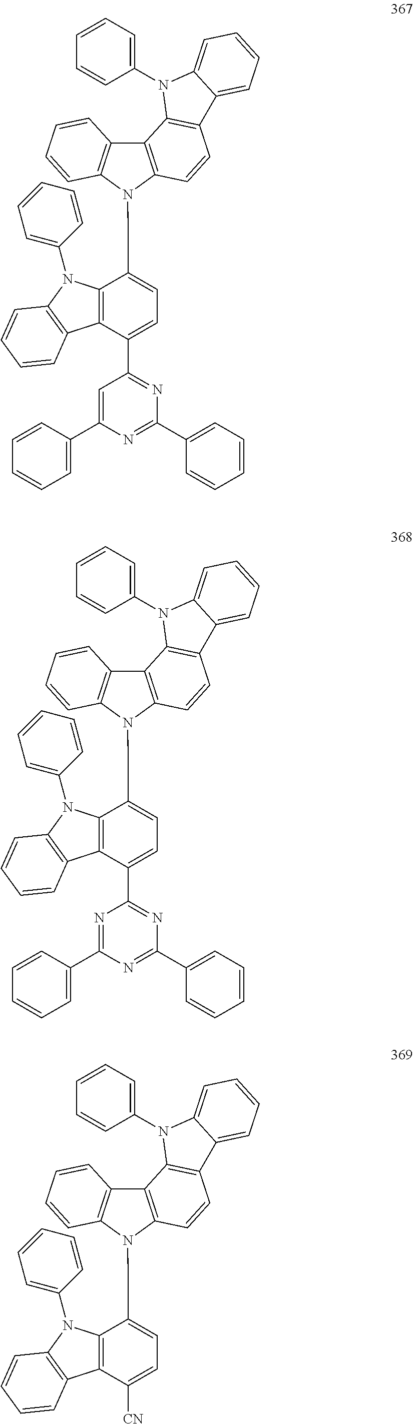 [SMILES: c1ccc(-c2cc(-c3ccc(-n4c5ccccc5c5c4ccc4c6ccccc6n(-c6ccccc6)c45)c4c3c3ccccc3n4-c3ccccc3)nc(-c3ccccc3)n2)cc1 ; c1ccc(-c2nc(-c3ccccc3)nc(-c3ccc(-n4c5ccccc5c5c4ccc4c6ccccc6n(-c6ccccc6)c45)c4c3c3ccccc3n4-c3ccccc3)n2)cc1 ; N#Cc1ccc(-n2c3ccccc3c3c2ccc2c4ccccc4n(-c4ccccc4)c23)c2c1c1ccccc1n2-c1ccccc1] 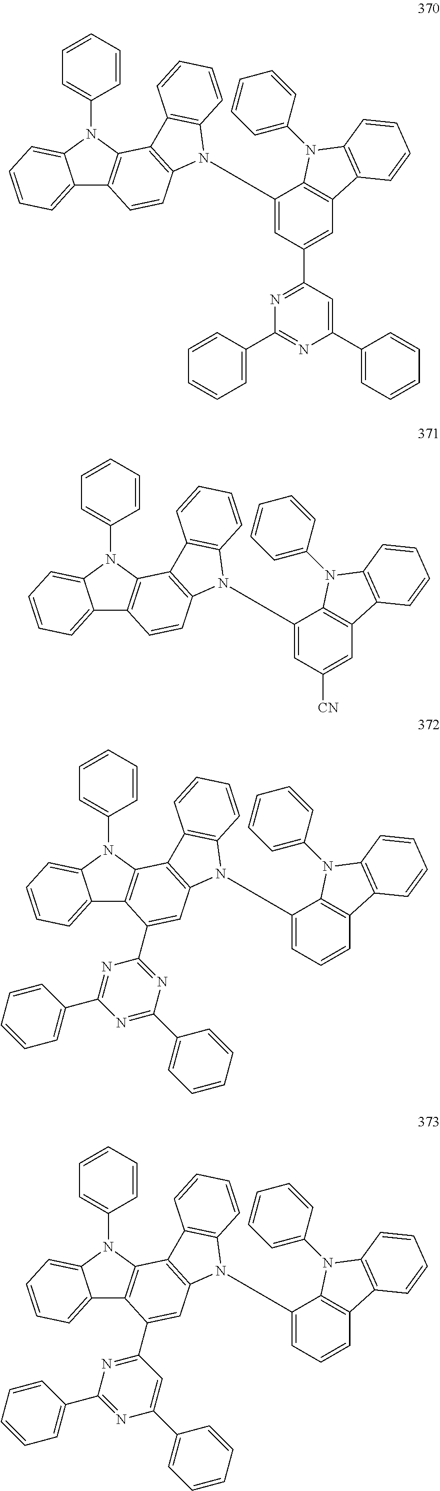 [SMILES: c1ccc(-c2cc(-c3cc(-n4c5ccccc5c5c4ccc4c6ccccc6n(-c6ccccc6)c45)c4c(c3)c3ccccc3n4-c3ccccc3)nc(-c3ccccc3)n2)cc1 ; N#Cc1cc(-n2c3ccccc3c3c2ccc2c4ccccc4n(-c4ccccc4)c23)c2c(c1)c1ccccc1n2-c1ccccc1 ; c1ccc(-c2nc(-c3ccccc3)nc(-c3cc4c(c5ccccc5n4-c4cccc5c6ccccc6n(-c6ccccc6)c45)c4c3c3ccccc3n4-c3ccccc3)n2)cc1 ; c1ccc(-c2cc(-c3cc4c(c5ccccc5n4-c4cccc5c6ccccc6n(-c6ccccc6)c45)c4c3c3ccccc3n4-c3ccccc3)nc(-c3ccccc3)n2)cc1] 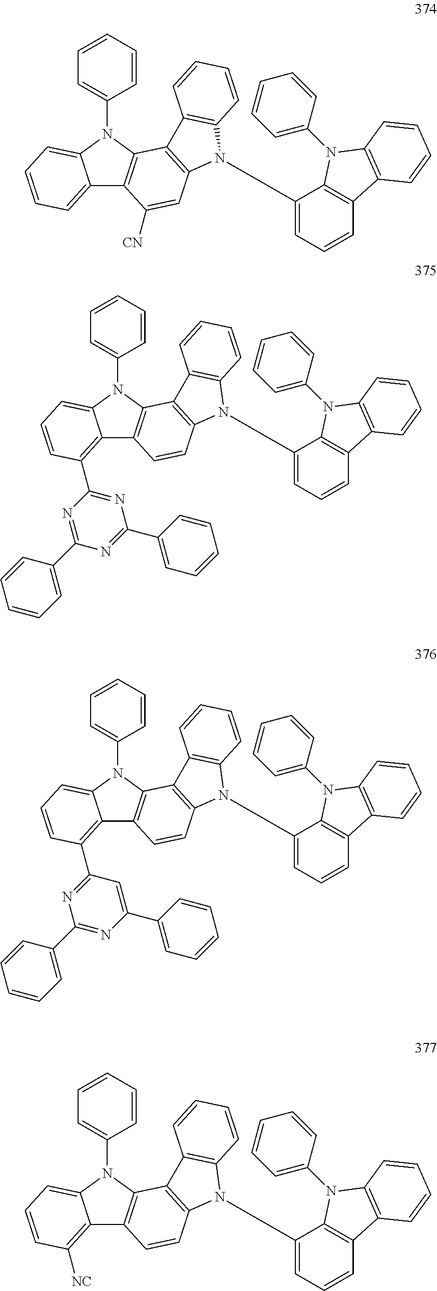 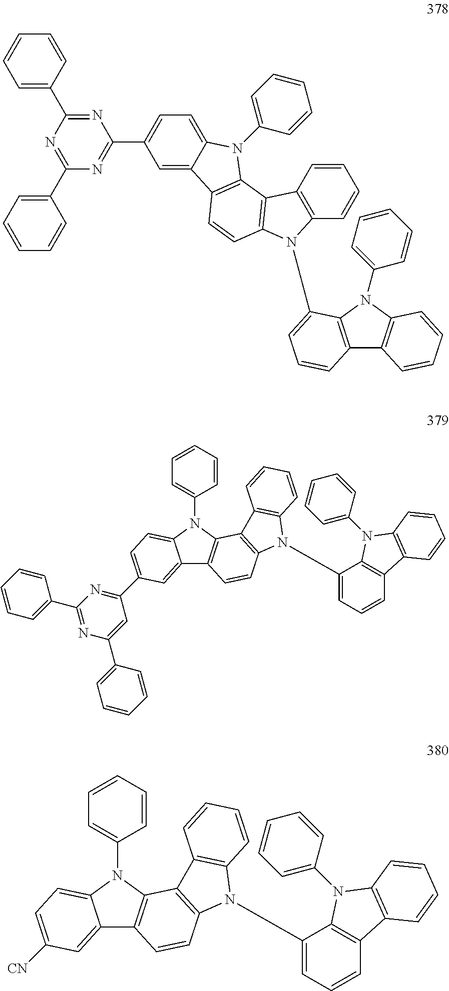 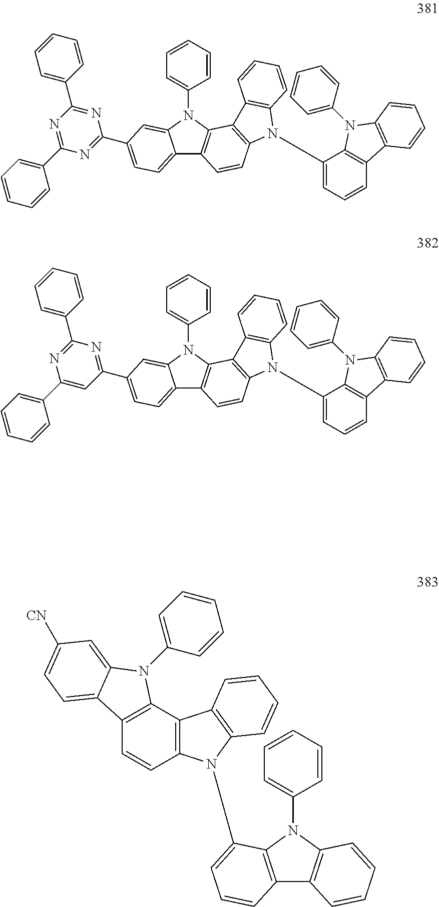 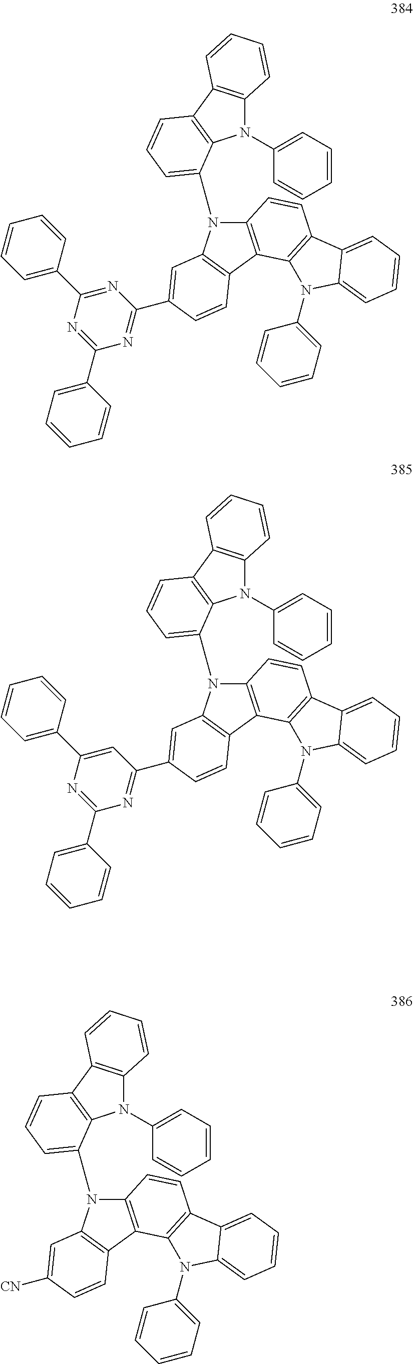 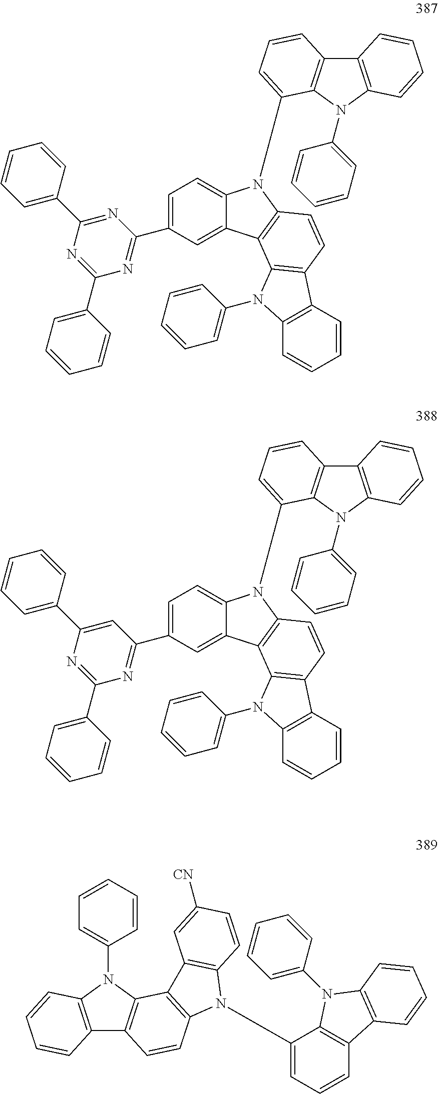 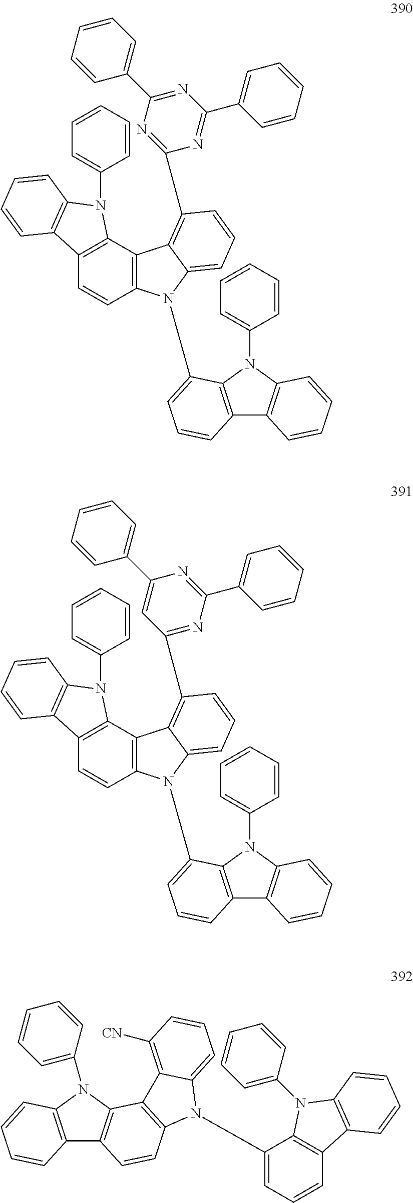 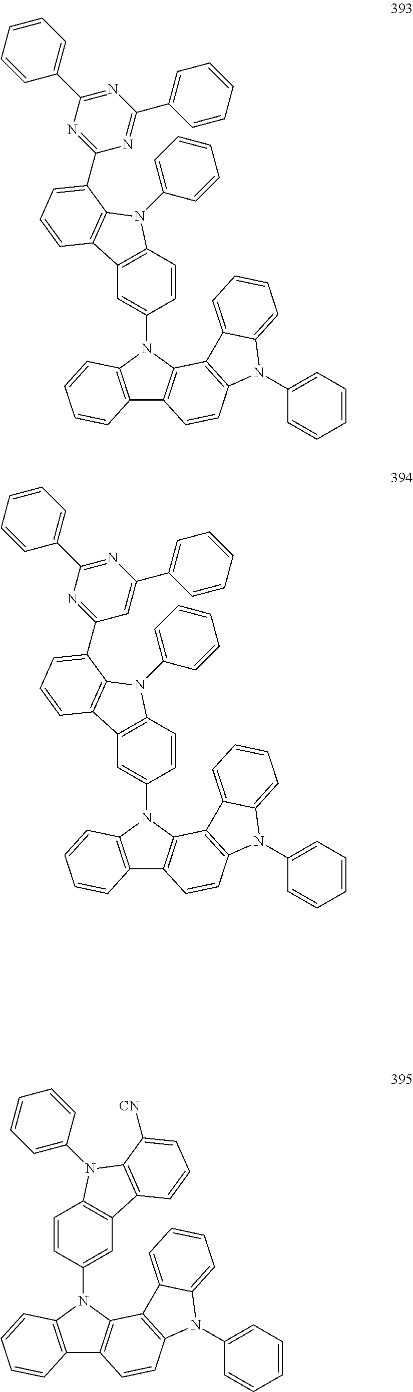 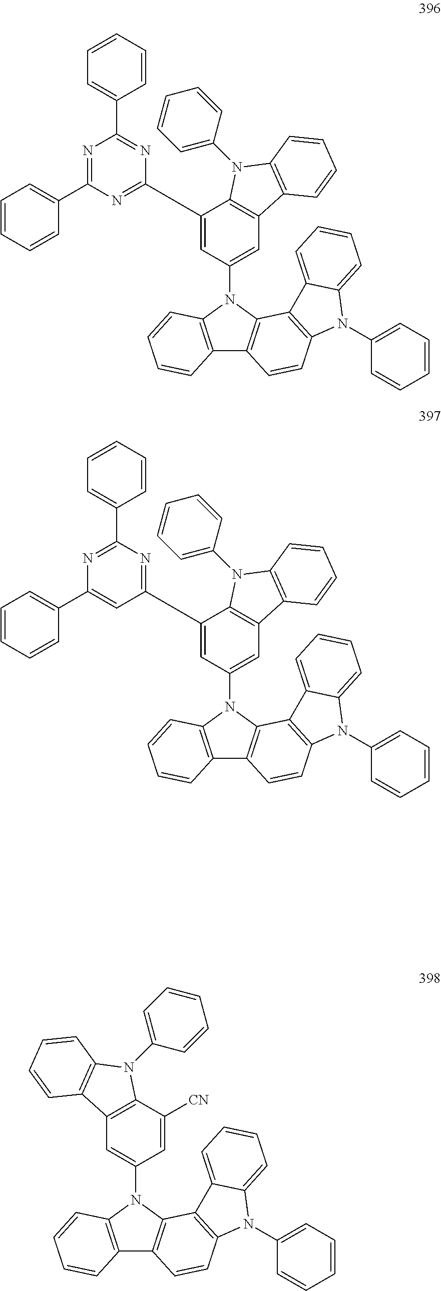 [SMILES: c1ccc(-c2nc(-c3ccccc3)nc(-c3cc(-n4c5ccccc5c5ccc6c(c7ccccc7n6-c6ccccc6)c54)cc4c5ccccc5n(-c5ccccc5)c34)n2)cc1 ; c1ccc(-c2cc(-c3cc(-n4c5ccccc5c5ccc6c(c7ccccc7n6-c6ccccc6)c54)cc4c5ccccc5n(-c5ccccc5)c34)nc(-c3ccccc3)n2)cc1 ; N#Cc1cc(-n2c3ccccc3c3ccc4c(c5ccccc5n4-c4ccccc4)c32)cc2c3ccccc3n(-c3ccccc3)c12] 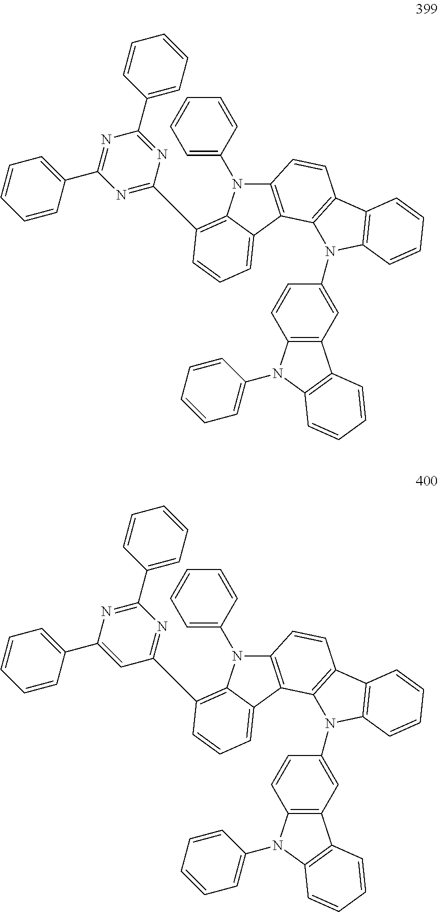 [SMILES: c1ccc(-c2nc(-c3ccccc3)nc(-c3cccc4c5c(ccc6c7ccccc7n(-c7ccc8c(c7)c7ccccc7n8-c7ccccc7)c65)n(-c5ccccc5)c34)n2)cc1 ; c1ccc(-c2cc(-c3cccc4c5c(ccc6c7ccccc7n(-c7ccc8c(c7)c7ccccc7n8-c7ccccc7)c65)n(-c5ccccc5)c34)nc(-c3ccccc3)n2)cc1] 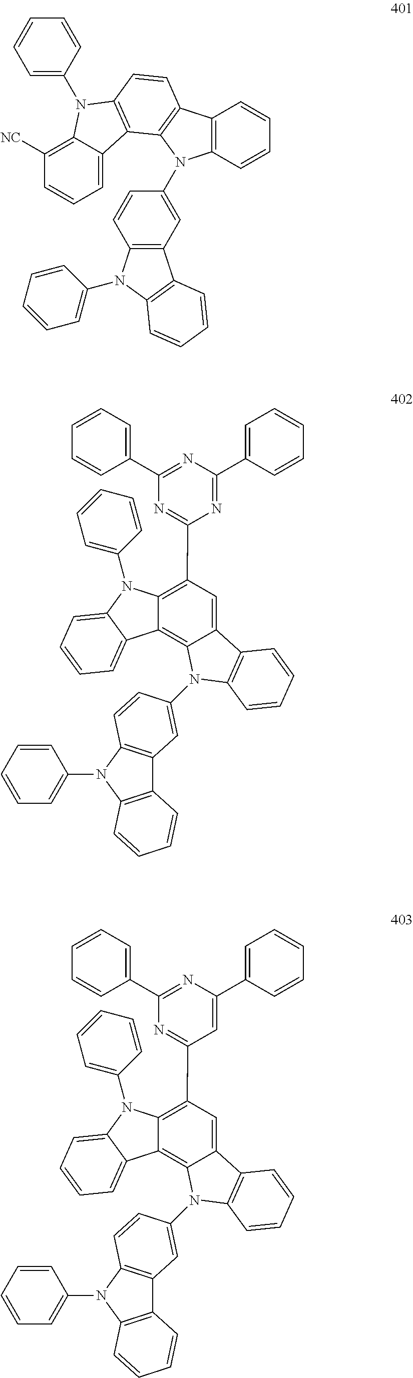 [SMILES: N#Cc1cccc2c3c(ccc4c5ccccc5n(-c5ccc6c(c5)c5ccccc5n6-c5ccccc5)c43)n(-c3ccccc3)c12 ; c1ccc(-c2nc(-c3ccccc3)nc(-c3cc4c5ccccc5n(-c5ccc6c(c5)c5ccccc5n6-c5ccccc5)c4c4c5ccccc5n(-c5ccccc5)c34)n2)cc1 ; c1ccc(-c2cc(-c3cc4c5ccccc5n(-c5ccc6c(c5)c5ccccc5n6-c5ccccc5)c4c4c5ccccc5n(-c5ccccc5)c34)nc(-c3ccccc3)n2)cc1] 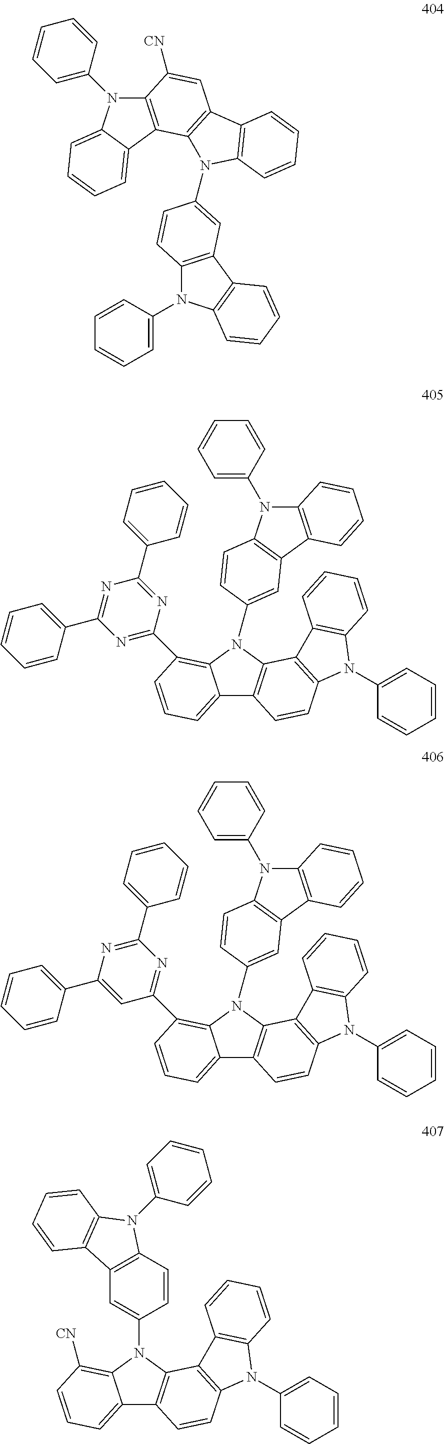 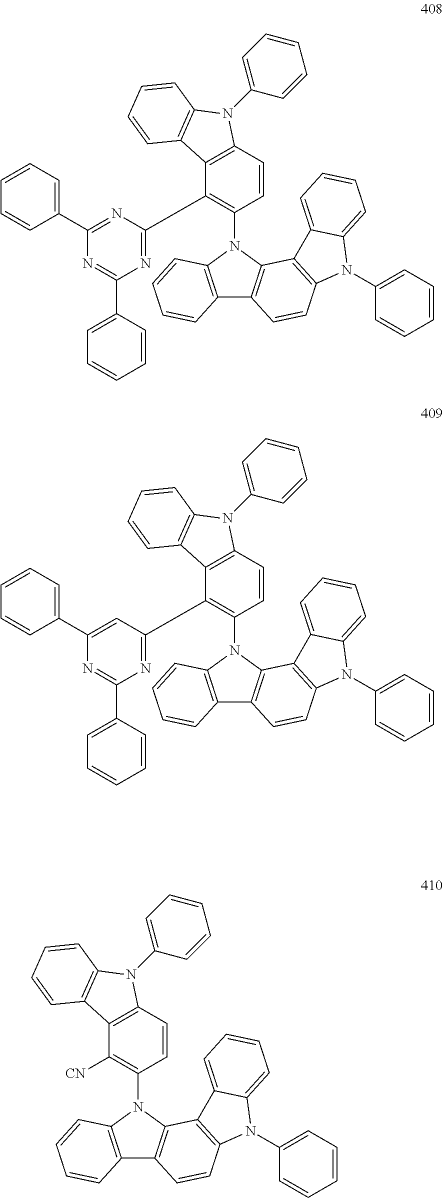 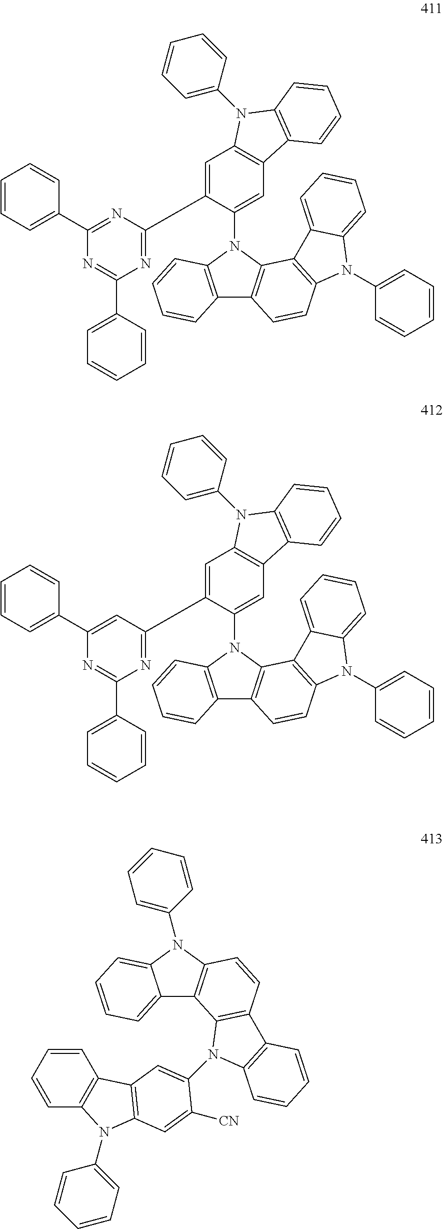 [SMILES: c1ccc(-c2nc(-c3ccccc3)nc(-c3cc4c(cc3-n3c5ccccc5c5ccc6c(c7ccccc7n6-c6ccccc6)c53)c3ccccc3n4-c3ccccc3)n2)cc1 ; c1ccc(-c2cc(-c3cc4c(cc3-n3c5ccccc5c5ccc6c(c7ccccc7n6-c6ccccc6)c53)c3ccccc3n4-c3ccccc3)nc(-c3ccccc3)n2)cc1 ; N#Cc1cc2c(cc1-n1c3ccccc3c3ccc4c(c5ccccc5n4-c4ccccc4)c31)c1ccccc1n2-c1ccccc1] 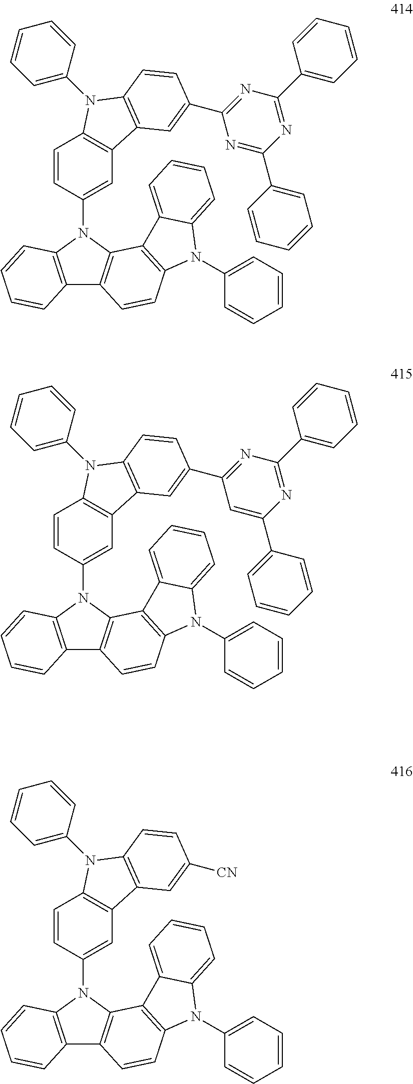 [SMILES: c1ccc(-c2nc(-c3ccccc3)nc(-c3ccc4c(c3)c3cc(-n5c6ccccc6c6ccc7c(c8ccccc8n7-c7ccccc7)c65)ccc3n4-c3ccccc3)n2)cc1 ; c1ccc(-c2cc(-c3ccc4c(c3)c3cc(-n5c6ccccc6c6ccc7c(c8ccccc8n7-c7ccccc7)c65)ccc3n4-c3ccccc3)nc(-c3ccccc3)n2)cc1 ; N#Cc1ccc2c(c1)c1cc(-n3c4ccccc4c4ccc5c(c6ccccc6n5-c5ccccc5)c43)ccc1n2-c1ccccc1] 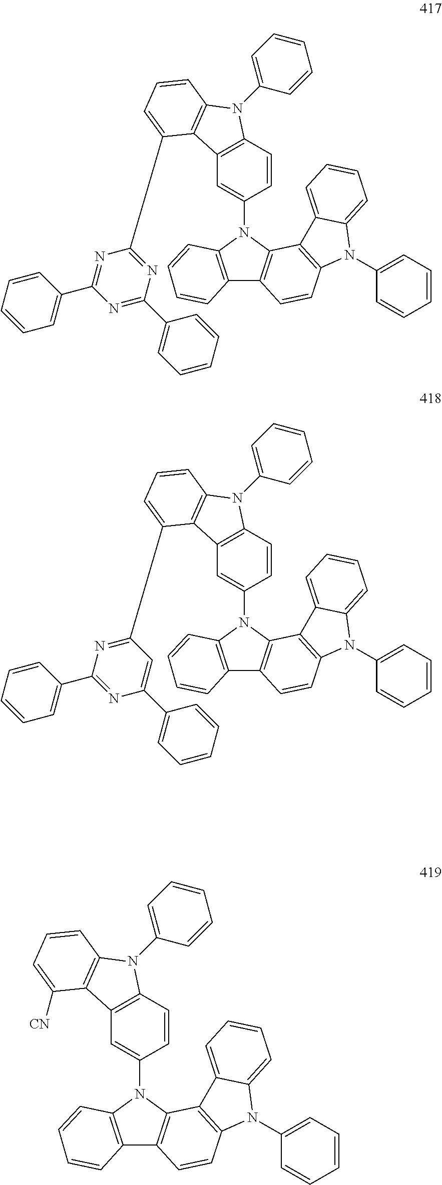 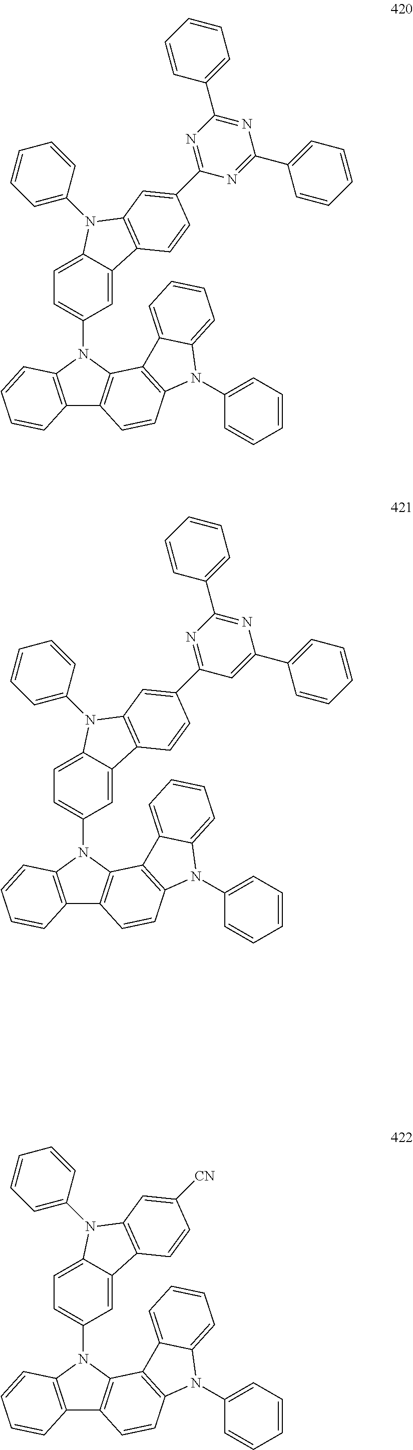 [SMILES: c1ccc(-c2nc(-c3ccccc3)nc(-c3ccc4c5cc(-n6c7ccccc7c7ccc8c(c9ccccc9n8-c8ccccc8)c76)ccc5n(-c5ccccc5)c4c3)n2)cc1 ; c1ccc(-c2cc(-c3ccc4c5cc(-n6c7ccccc7c7ccc8c(c9ccccc9n8-c8ccccc8)c76)ccc5n(-c5ccccc5)c4c3)nc(-c3ccccc3)n2)cc1 ; N#Cc1ccc2c3cc(-n4c5ccccc5c5ccc6c(c7ccccc7n6-c6ccccc6)c54)ccc3n(-c3ccccc3)c2c1] 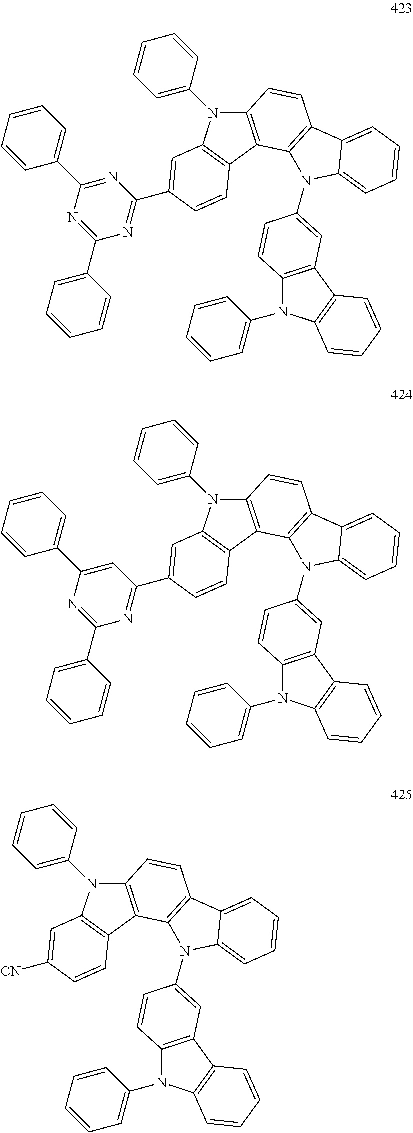 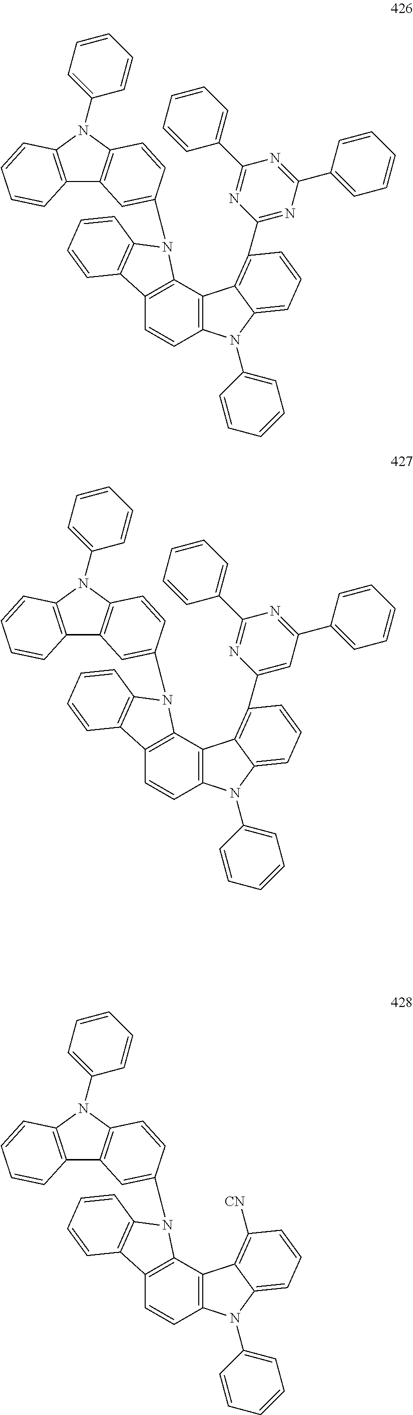 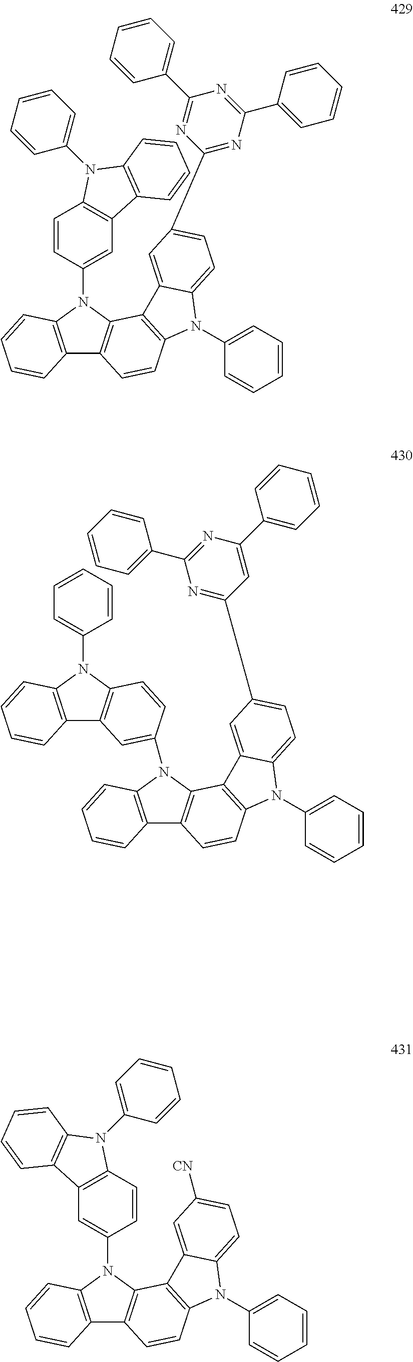 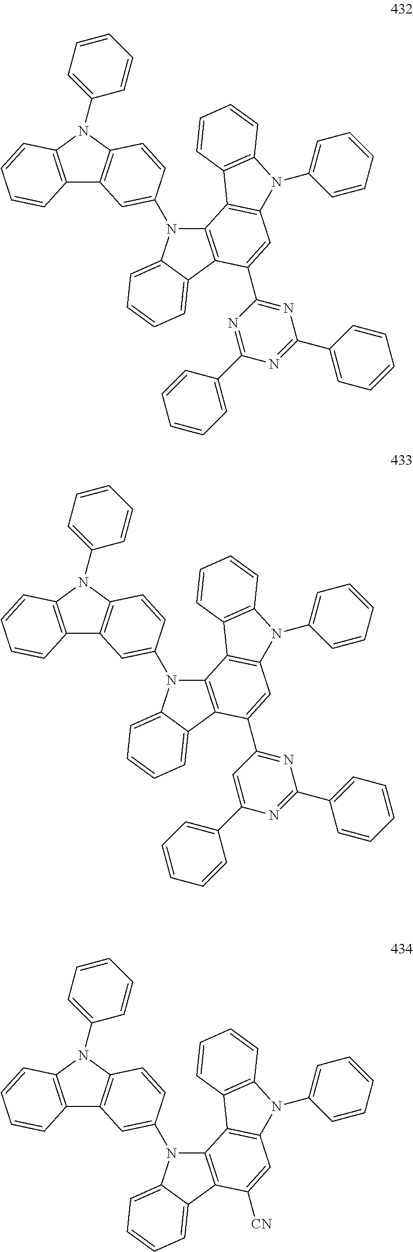 [SMILES: c1ccc(-c2nc(-c3ccccc3)nc(-c3cc4c(c5ccccc5n4-c4ccccc4)c4c3c3ccccc3n4-c3ccc4c(c3)c3ccccc3n4-c3ccccc3)n2)cc1 ; c1ccc(-c2cc(-c3cc4c(c5ccccc5n4-c4ccccc4)c4c3c3ccccc3n4-c3ccc4c(c3)c3ccccc3n4-c3ccccc3)nc(-c3ccccc3)n2)cc1 ; N#Cc1cc2c(c3ccccc3n2-c2ccccc2)c2c1c1ccccc1n2-c1ccc2c(c1)c1ccccc1n2-c1ccccc1] 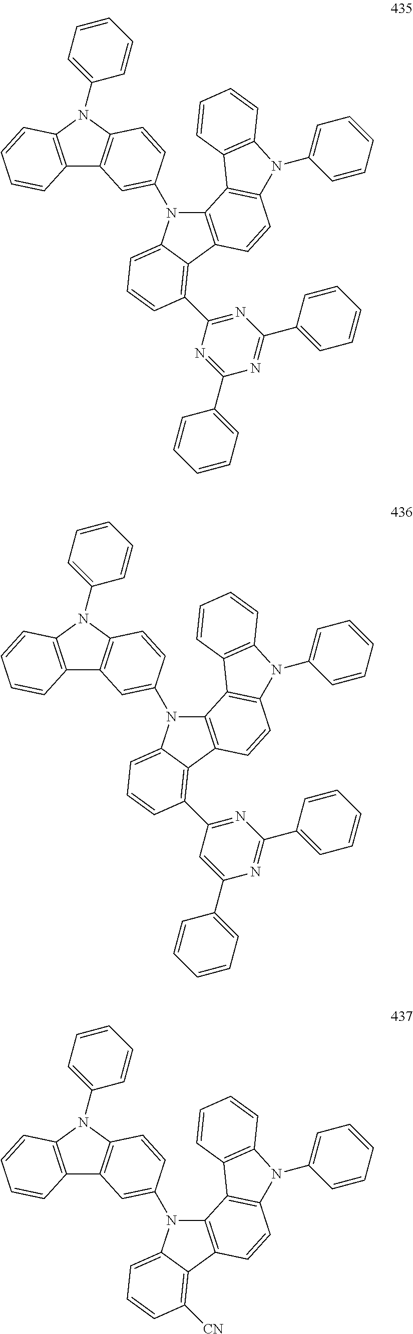 [SMILES: c1ccc(-c2nc(-c3ccccc3)nc(-c3cccc4c3c3ccc5c(c6ccccc6n5-c5ccccc5)c3n4-c3ccc4c(c3)c3ccccc3n4-c3ccccc3)n2)cc1 ; c1ccc(-c2cc(-c3cccc4c3c3ccc5c(c6ccccc6n5-c5ccccc5)c3n4-c3ccc4c(c3)c3ccccc3n4-c3ccccc3)nc(-c3ccccc3)n2)cc1 ; N#Cc1cccc2c1c1ccc3c(c4ccccc4n3-c3ccccc3)c1n2-c1ccc2c(c1)c1ccccc1n2-c1ccccc1] 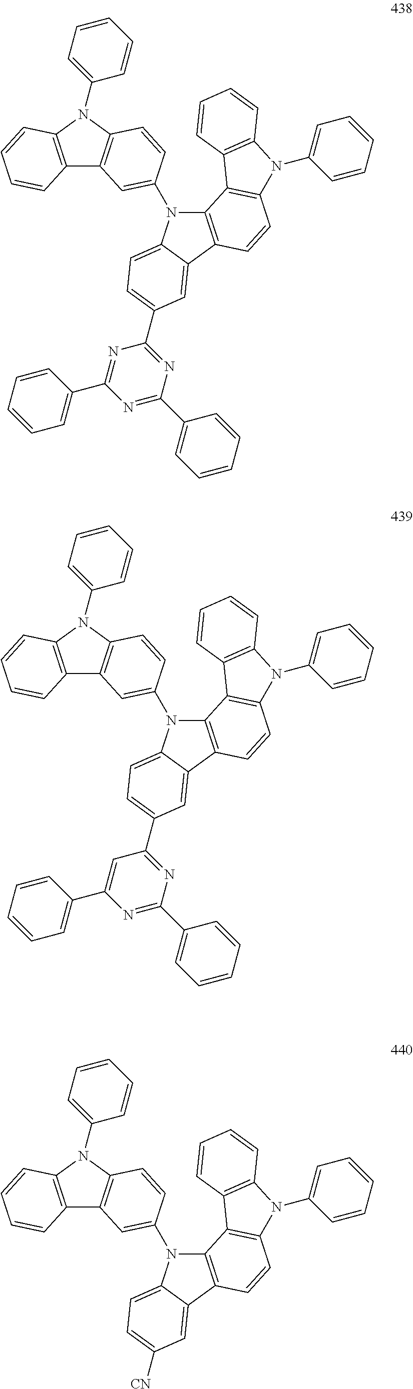 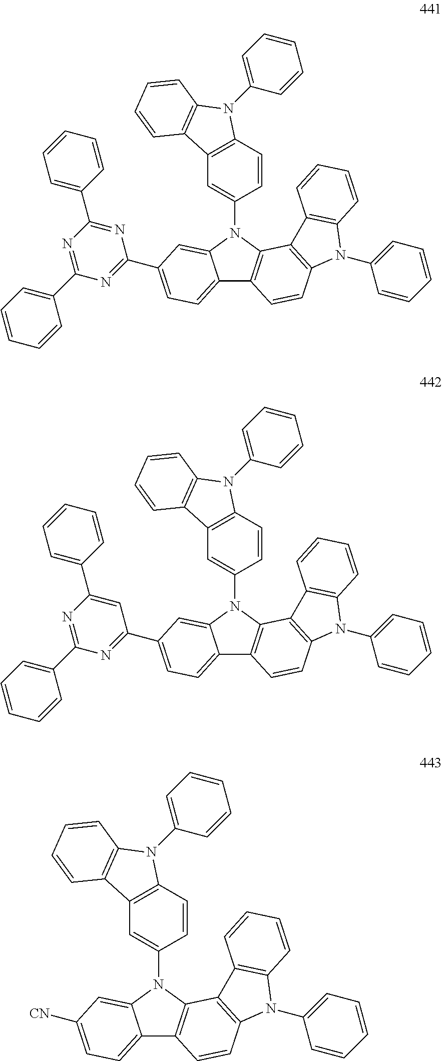 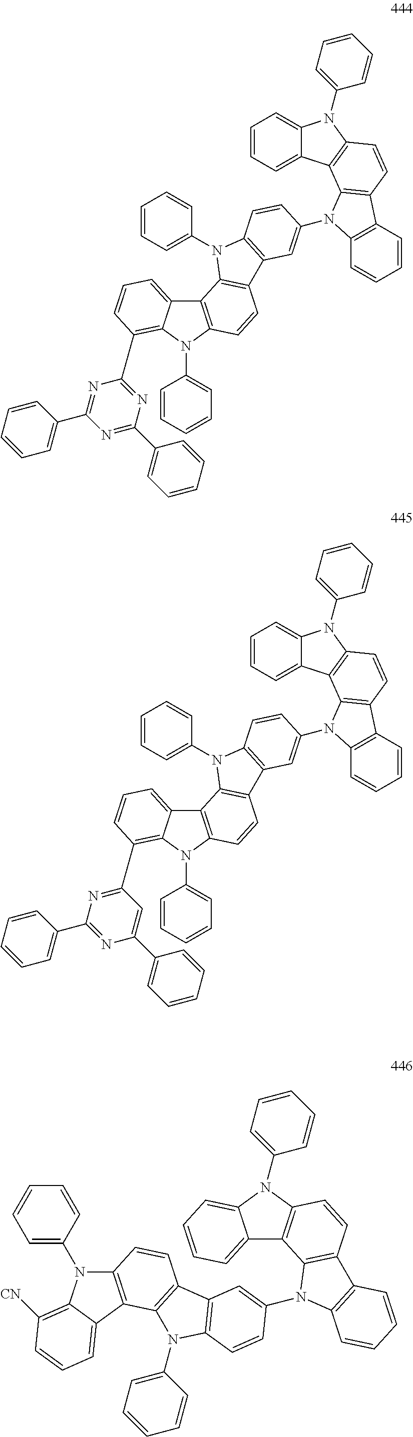 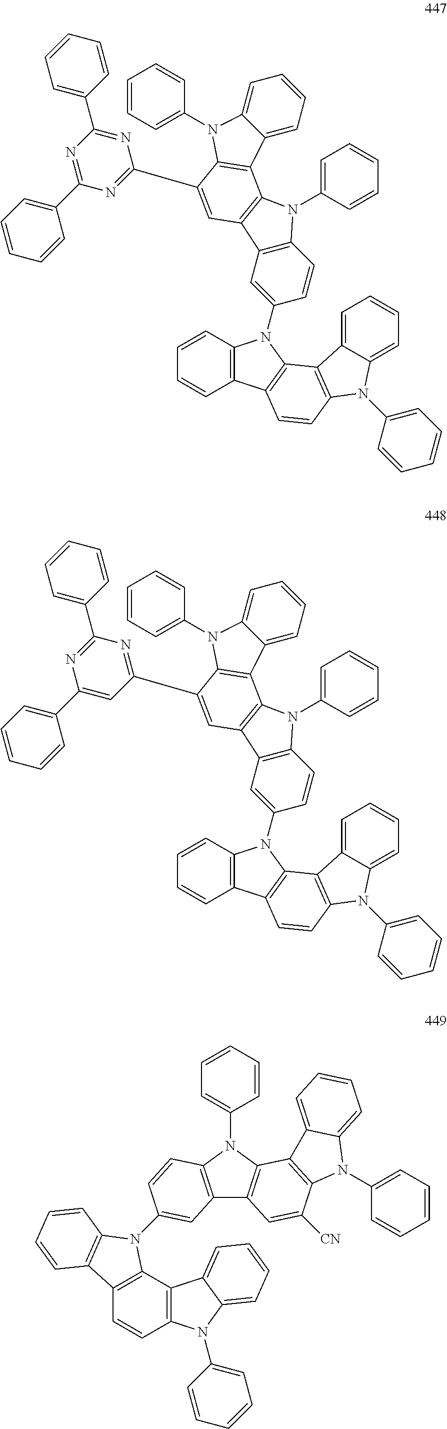 [SMILES: c1ccc(-c2nc(-c3ccccc3)nc(-c3cc4c5cc(-n6c7ccccc7c7ccc8c(c9ccccc9n8-c8ccccc8)c76)ccc5n(-c5ccccc5)c4c4c5ccccc5n(-c5ccccc5)c34)n2)cc1 ; c1ccc(-c2cc(-c3cc4c5cc(-n6c7ccccc7c7ccc8c(c9ccccc9n8-c8ccccc8)c76)ccc5n(-c5ccccc5)c4c4c5ccccc5n(-c5ccccc5)c34)nc(-c3ccccc3)n2)cc1 ; N#Cc1cc2c3cc(-n4c5ccccc5c5ccc6c(c7ccccc7n6-c6ccccc6)c54)ccc3n(-c3ccccc3)c2c2c3ccccc3n(-c3ccccc3)c12] 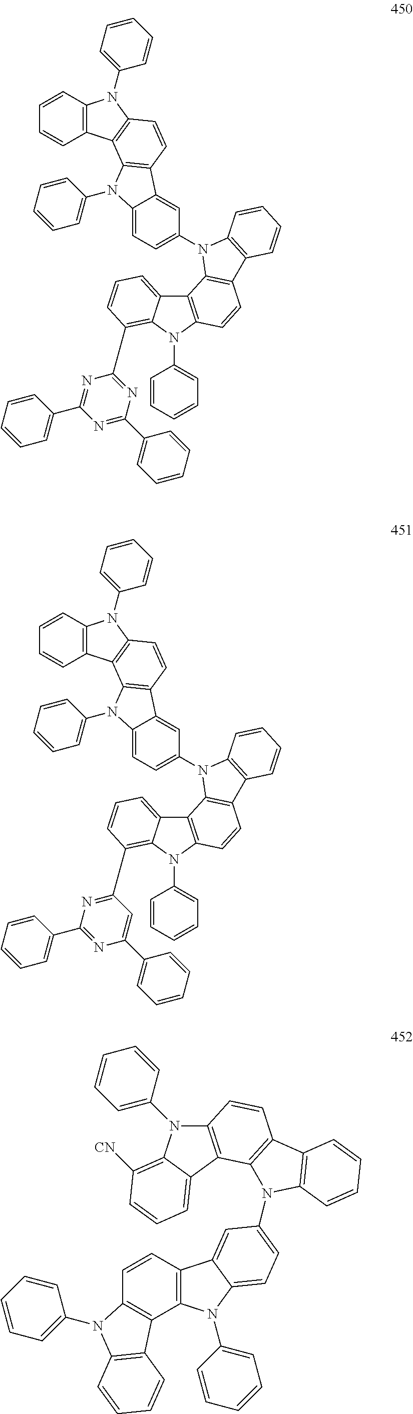 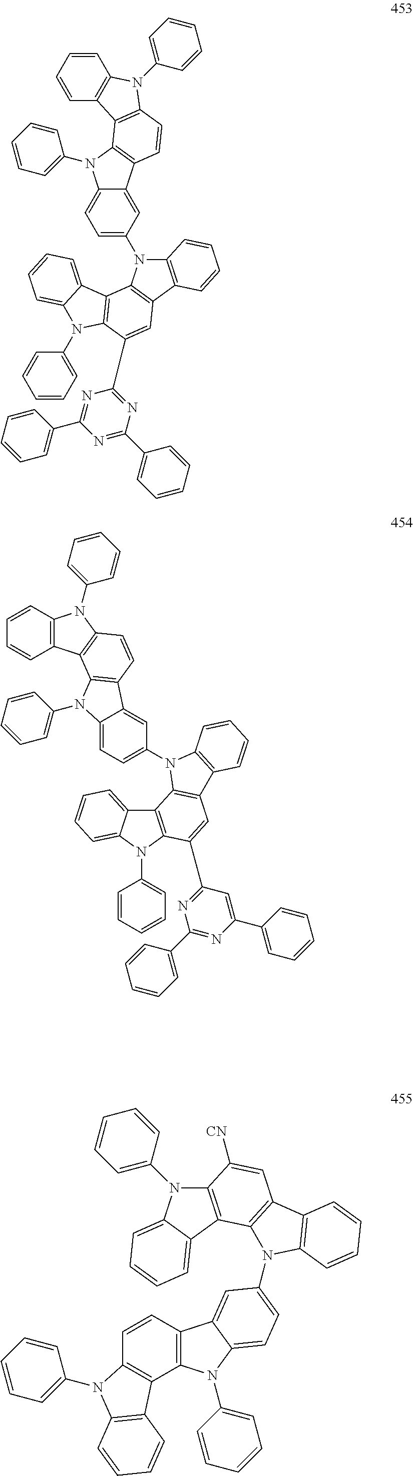 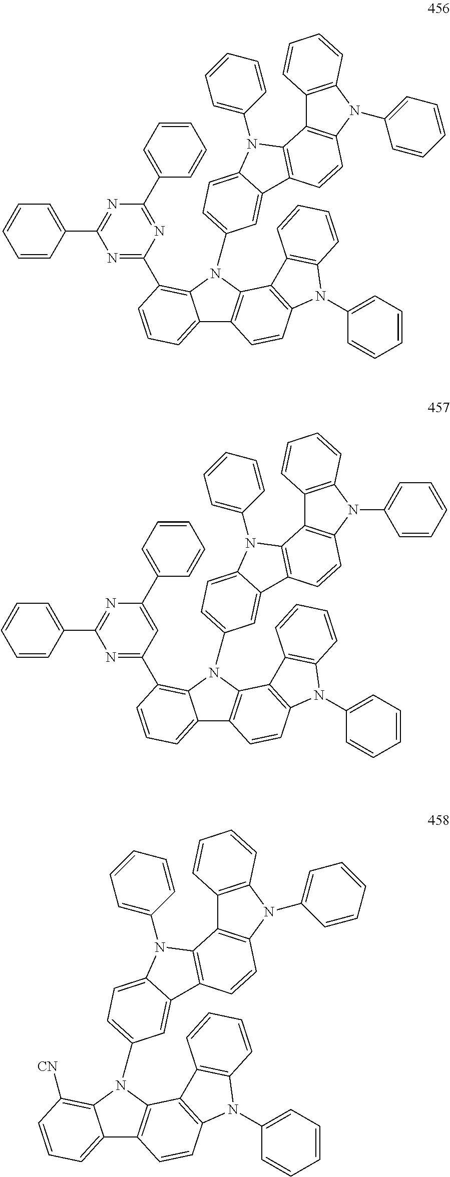 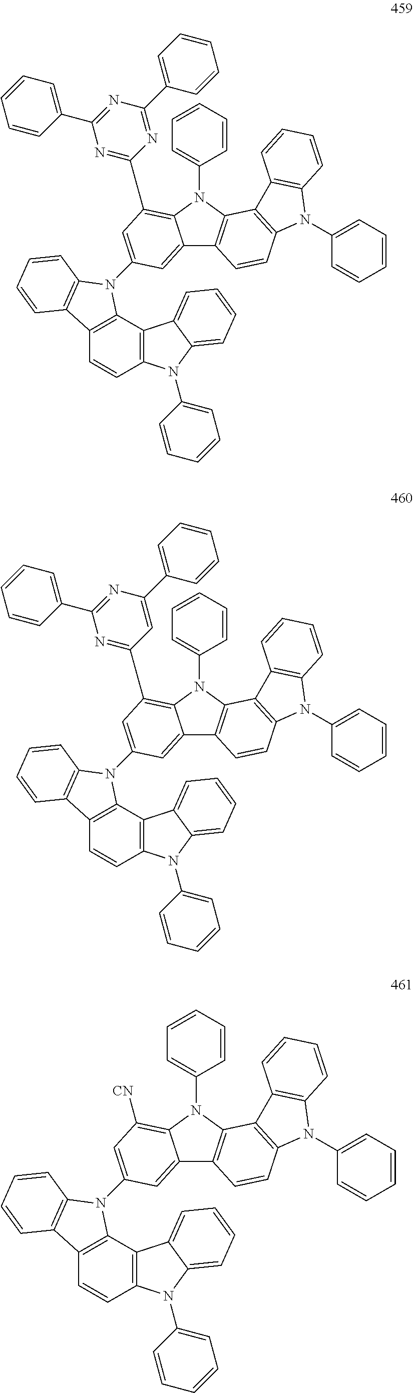 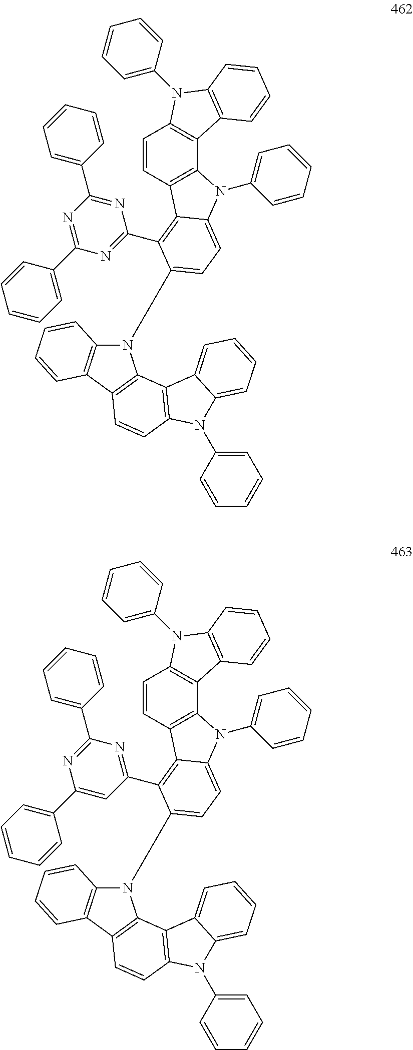 [SMILES: c1ccc(-c2nc(-c3ccccc3)nc(-c3c(-n4c5ccccc5c5ccc6c(c7ccccc7n6-c6ccccc6)c54)ccc4c3c3ccc5c(c6ccccc6n5-c5ccccc5)c3n4-c3ccccc3)n2)cc1 ; c1ccc(-c2cc(-c3c(-n4c5ccccc5c5ccc6c(c7ccccc7n6-c6ccccc6)c54)ccc4c3c3ccc5c(c6ccccc6n5-c5ccccc5)c3n4-c3ccccc3)nc(-c3ccccc3)n2)cc1] 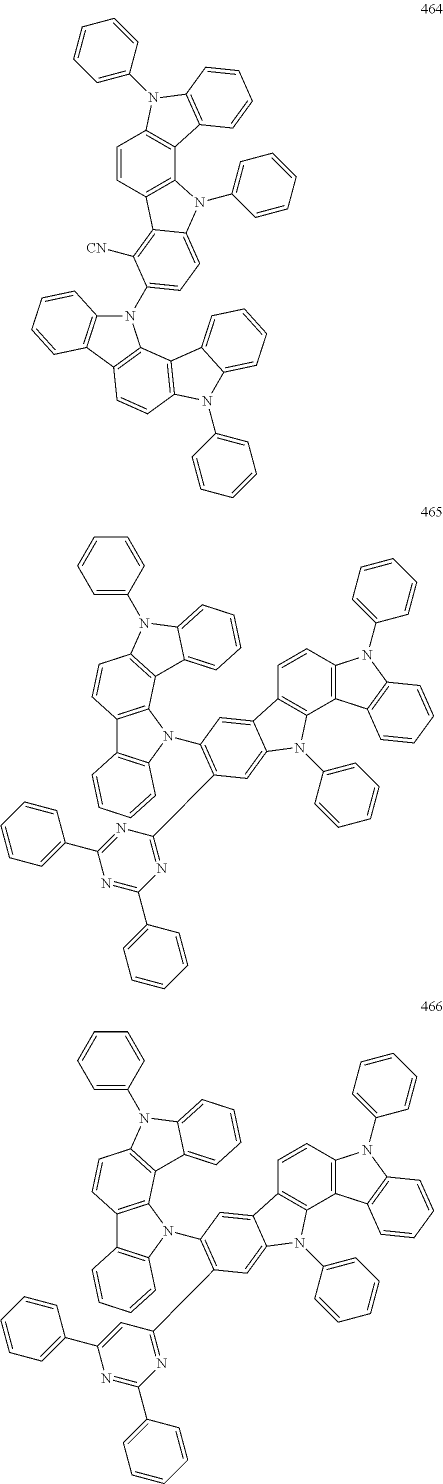 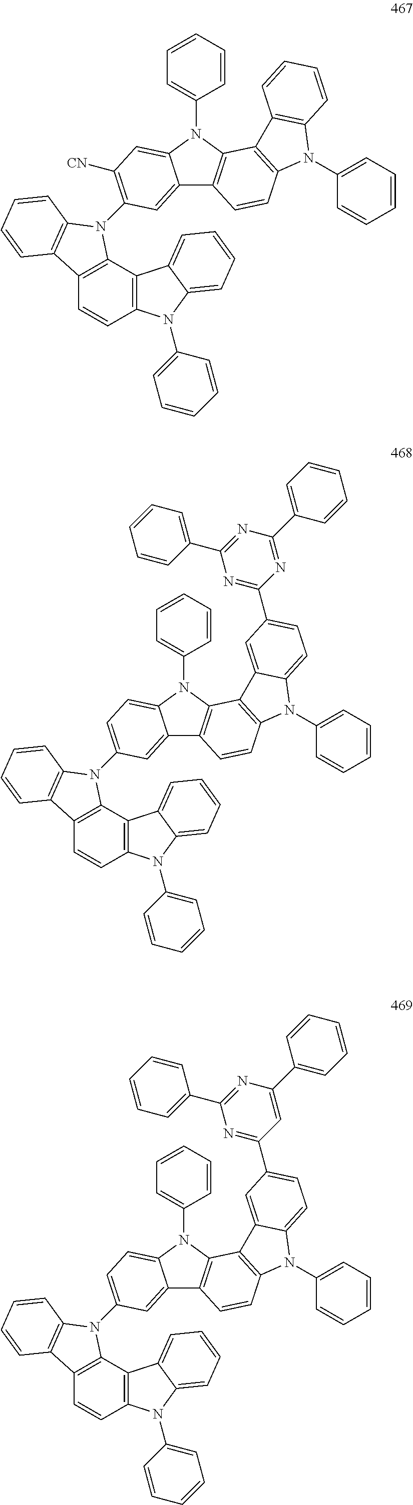 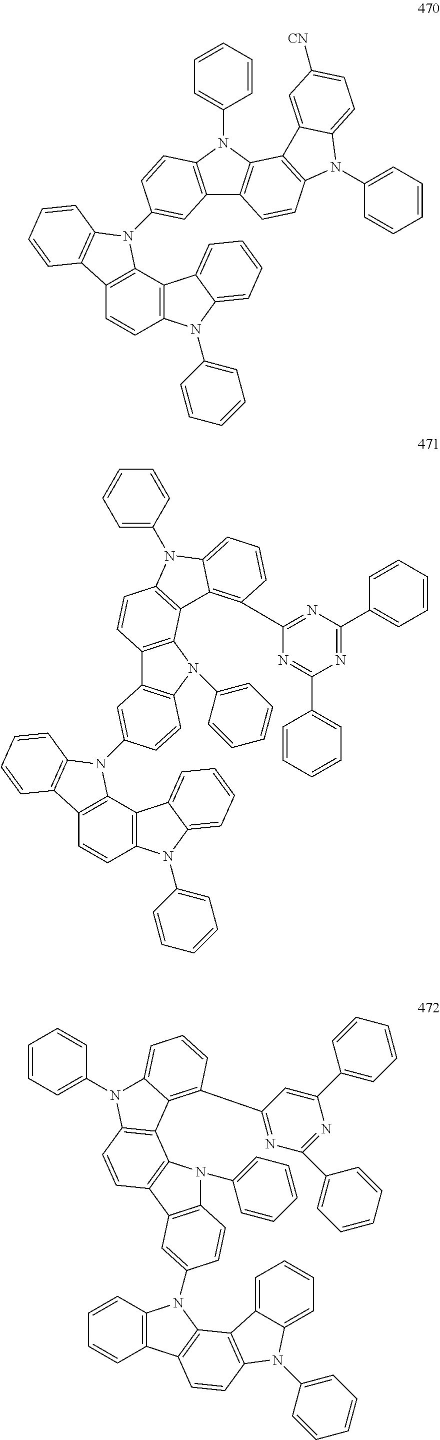 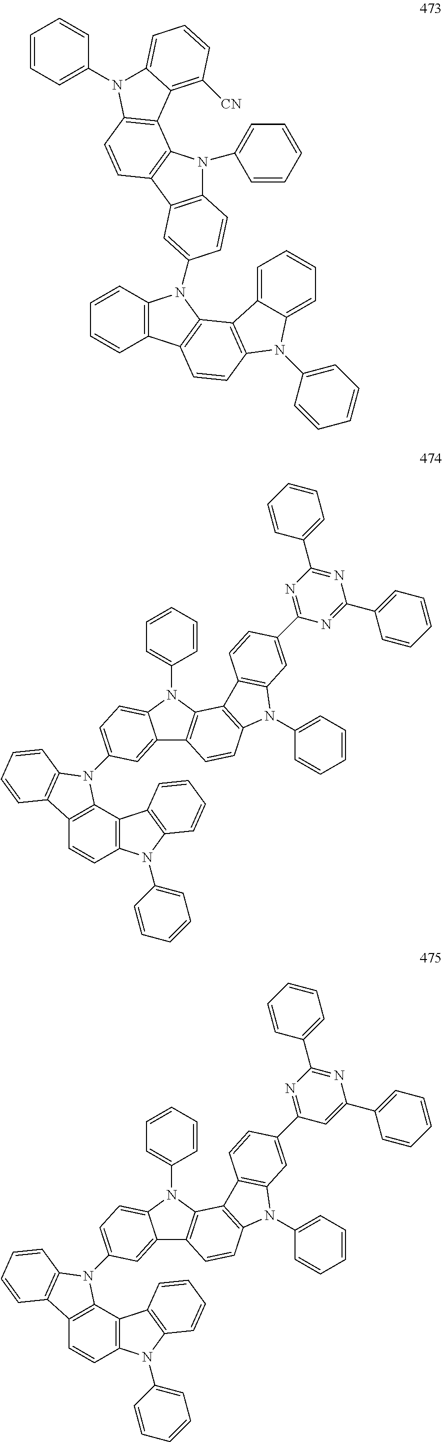 [SMILES: N#Cc1cccc2c1c1c(ccc3c4cc(-n5c6ccccc6c6ccc7c(c8ccccc8n7-c7ccccc7)c65)ccc4n(-c4ccccc4)c31)n2-c1ccccc1 ; c1ccc(-c2nc(-c3ccccc3)nc(-c3ccc4c5c(ccc6c7cc(-n8c9ccccc9c9ccc%10c(c%11ccccc%11n%10-c%10ccccc%10)c98)ccc7n(-c7ccccc7)c65)n(-c5ccccc5)c4c3)n2)cc1 ; c1ccc(-c2cc(-c3ccc4c5c(ccc6c7cc(-n8c9ccccc9c9ccc%10c(c%11ccccc%11n%10-c%10ccccc%10)c98)ccc7n(-c7ccccc7)c65)n(-c5ccccc5)c4c3)nc(-c3ccccc3)n2)cc1] 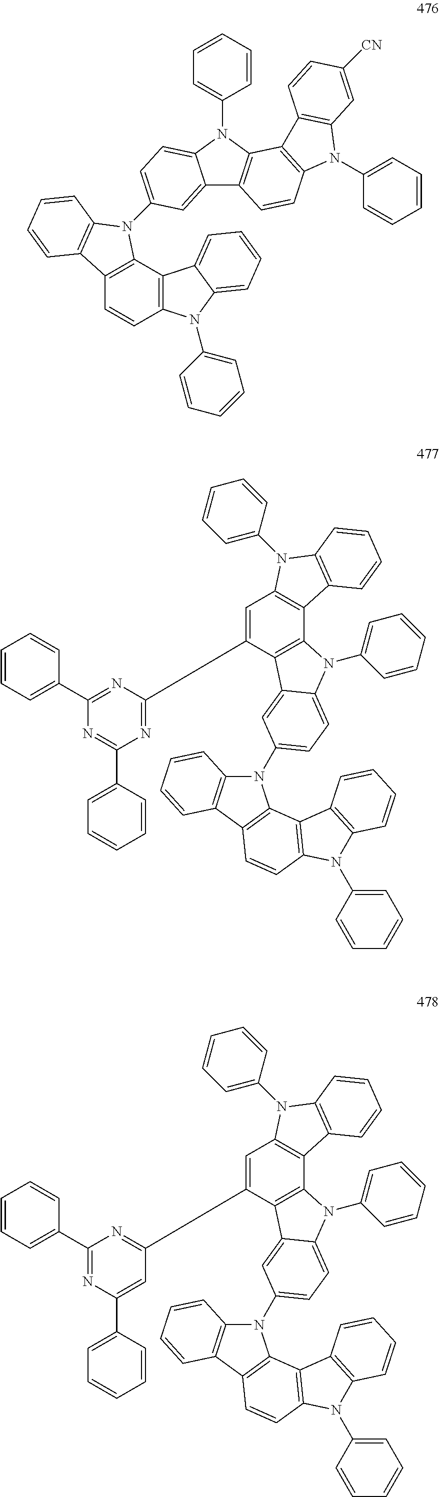 [SMILES: N#Cc1ccc2c3c(ccc4c5cc(-n6c7ccccc7c7ccc8c(c9ccccc9n8-c8ccccc8)c76)ccc5n(-c5ccccc5)c43)n(-c3ccccc3)c2c1 ; c1ccc(-c2nc(-c3ccccc3)nc(-c3cc4c(c5ccccc5n4-c4ccccc4)c4c3c3cc(-n5c6ccccc6c6ccc7c(c8ccccc8n7-c7ccccc7)c65)ccc3n4-c3ccccc3)n2)cc1 ; c1ccc(-c2cc(-c3cc4c(c5ccccc5n4-c4ccccc4)c4c3c3cc(-n5c6ccccc6c6ccc7c(c8ccccc8n7-c7ccccc7)c65)ccc3n4-c3ccccc3)nc(-c3ccccc3)n2)cc1] 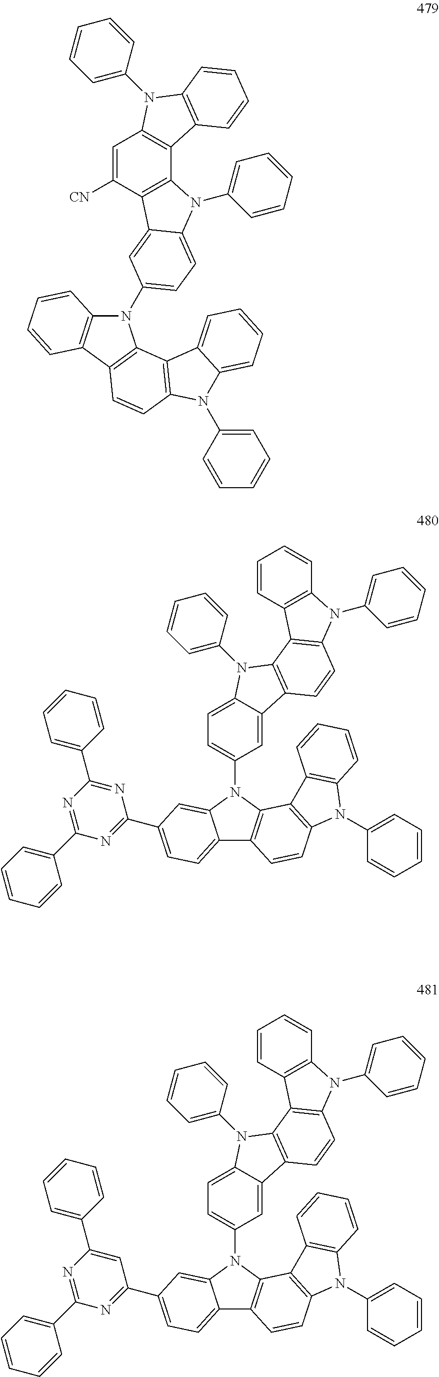 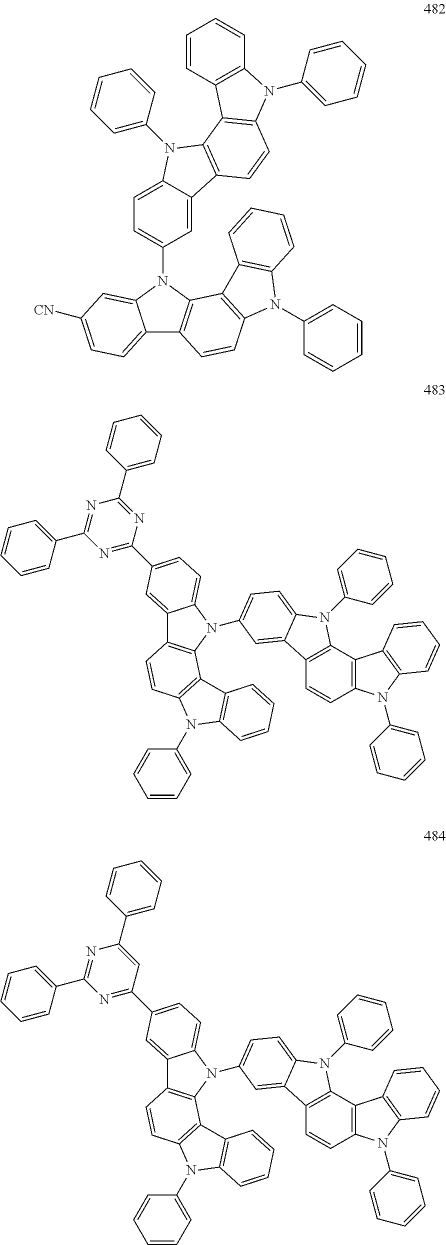 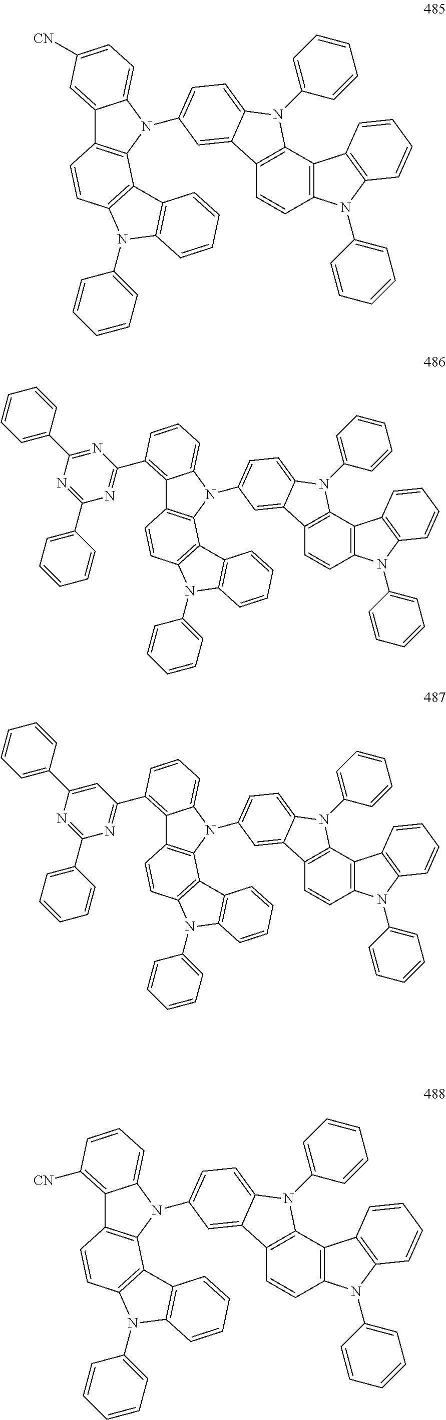 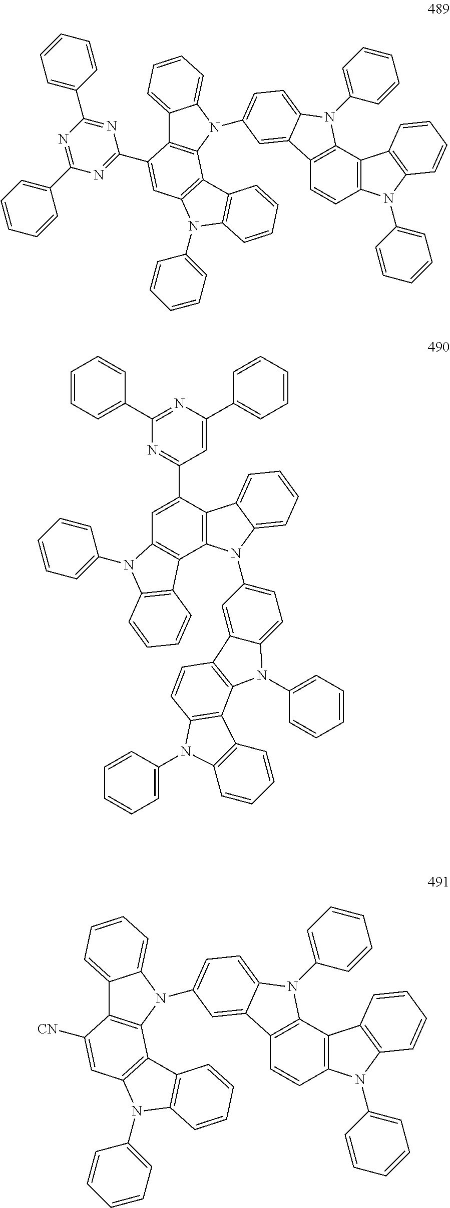 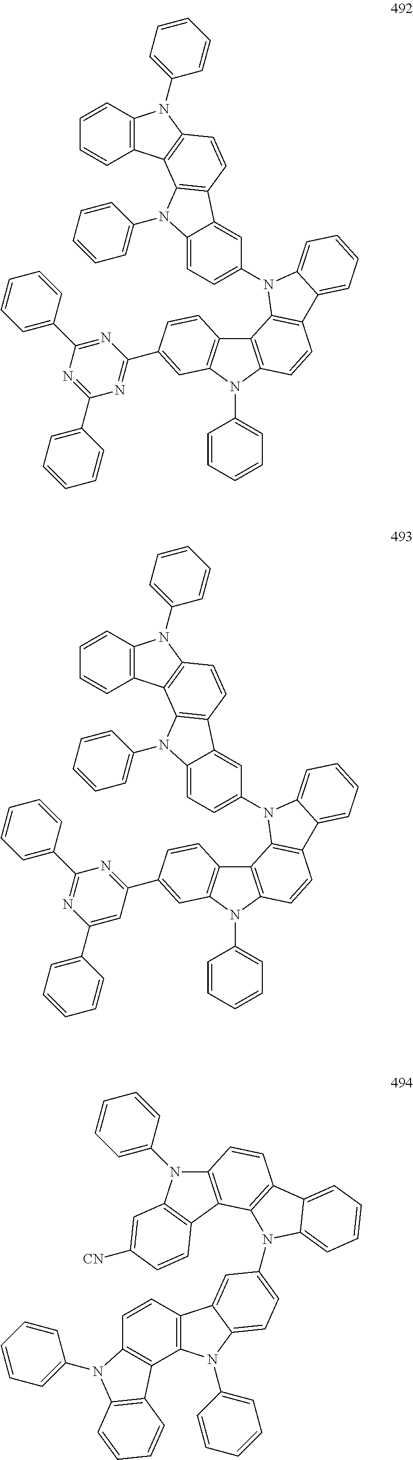 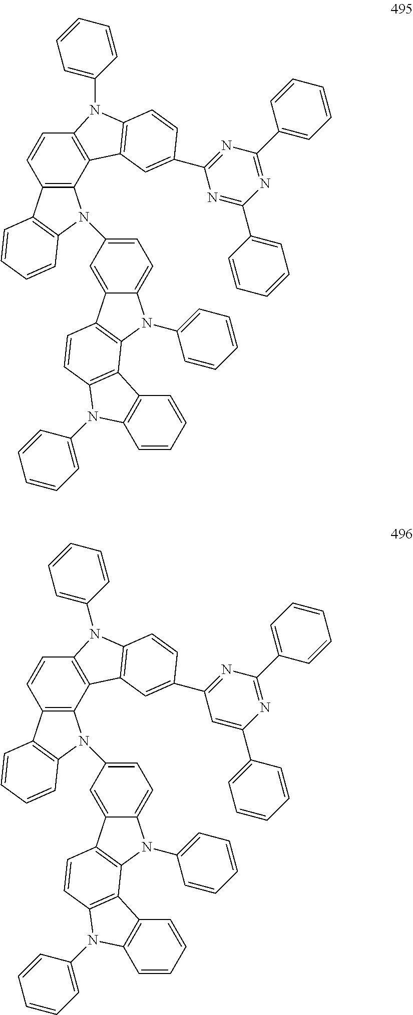 [SMILES: c1ccc(-c2nc(-c3ccccc3)nc(-c3ccc4c(c3)c3c(ccc5c6ccccc6n(-c6ccc7c(c6)c6ccc8c(c9ccccc9n8-c8ccccc8)c6n7-c6ccccc6)c53)n4-c3ccccc3)n2)cc1 ; c1ccc(-c2cc(-c3ccc4c(c3)c3c(ccc5c6ccccc6n(-c6ccc7c(c6)c6ccc8c(c9ccccc9n8-c8ccccc8)c6n7-c6ccccc6)c53)n4-c3ccccc3)nc(-c3ccccc3)n2)cc1] 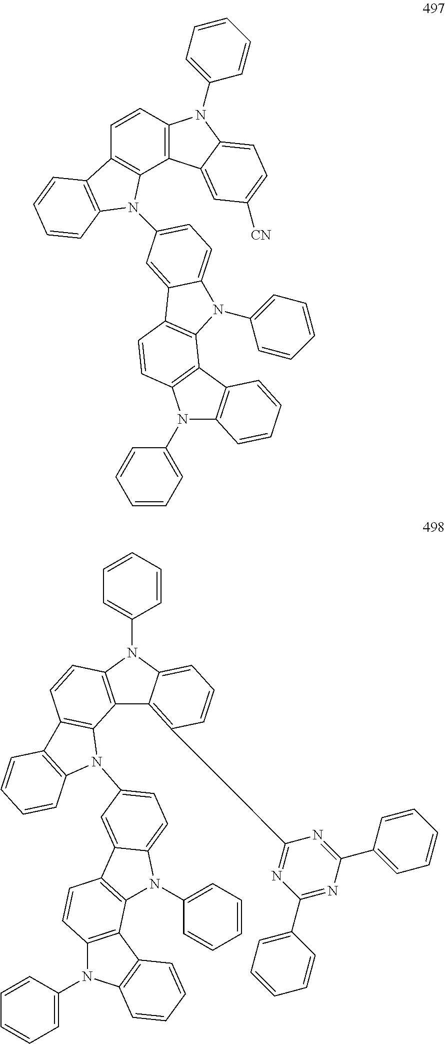 [SMILES: N#Cc1ccc2c(c1)c1c(ccc3c4ccccc4n(-c4ccc5c(c4)c4ccc6c(c7ccccc7n6-c6ccccc6)c4n5-c4ccccc4)c31)n2-c1ccccc1 ; c1ccc(-c2nc(-c3ccccc3)nc(-c3cccc4c3c3c(ccc5c6ccccc6n(-c6ccc7c(c6)c6ccc8c(c9ccccc9n8-c8ccccc8)c6n7-c6ccccc6)c53)n4-c3ccccc3)n2)cc1] 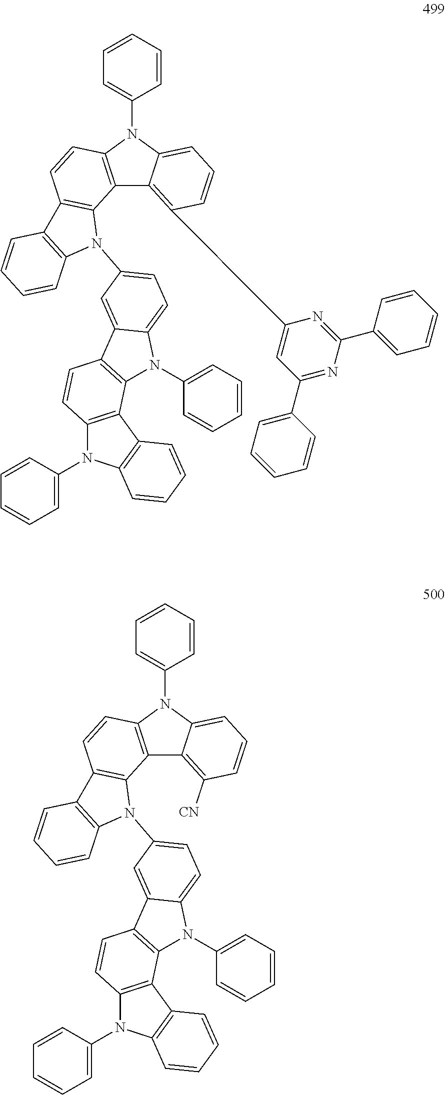 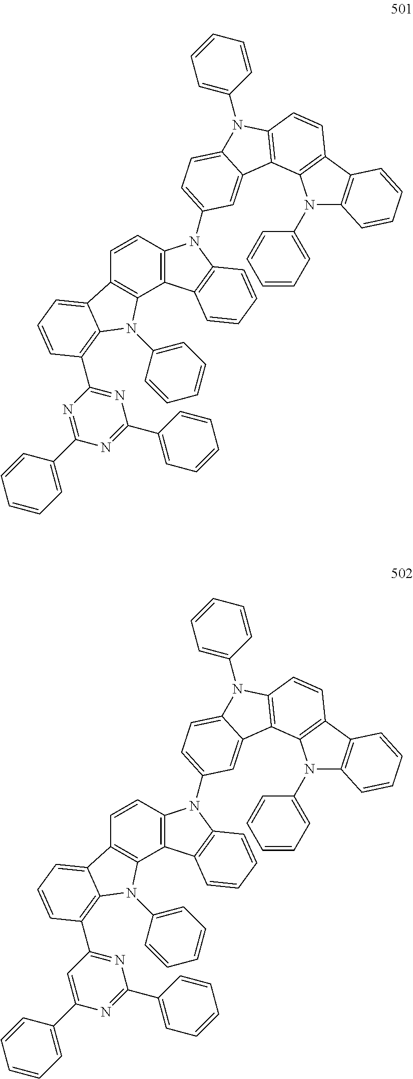 [SMILES: c1ccc(-c2nc(-c3ccccc3)nc(-c3cccc4c5ccc6c(c7ccccc7n6-c6ccc7c(c6)c6c(ccc8c9ccccc9n(-c9ccccc9)c86)n7-c6ccccc6)c5n(-c5ccccc5)c34)n2)cc1 ; c1ccc(-c2cc(-c3cccc4c5ccc6c(c7ccccc7n6-c6ccc7c(c6)c6c(ccc8c9ccccc9n(-c9ccccc9)c86)n7-c6ccccc6)c5n(-c5ccccc5)c34)nc(-c3ccccc3)n2)cc1] 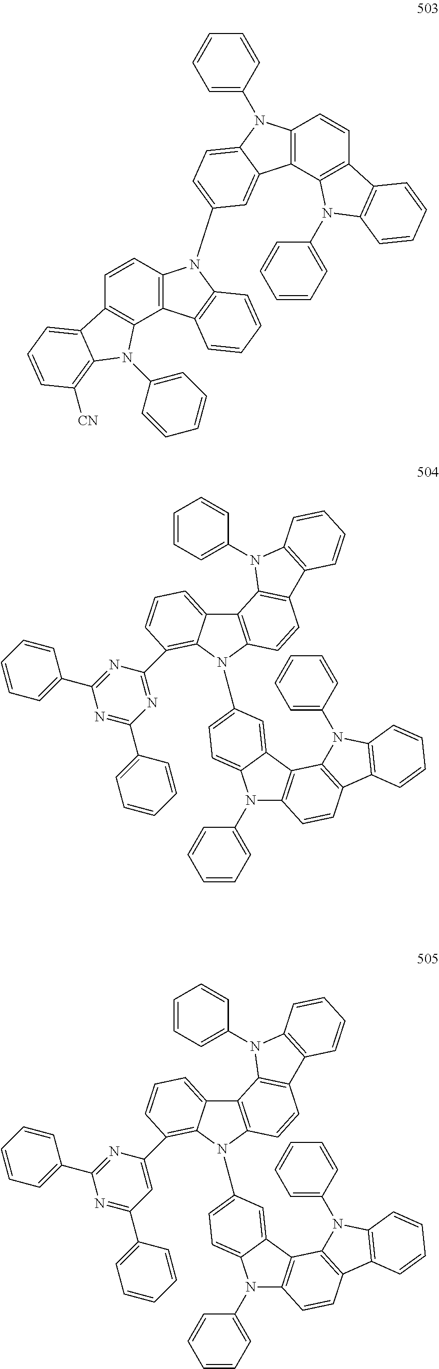 [SMILES: N#Cc1cccc2c3ccc4c(c5ccccc5n4-c4ccc5c(c4)c4c(ccc6c7ccccc7n(-c7ccccc7)c64)n5-c4ccccc4)c3n(-c3ccccc3)c12 ; c1ccc(-c2nc(-c3ccccc3)nc(-c3cccc4c5c(ccc6c7ccccc7n(-c7ccccc7)c65)n(-c5ccc6c(c5)c5c(ccc7c8ccccc8n(-c8ccccc8)c75)n6-c5ccccc5)c34)n2)cc1 ; c1ccc(-c2cc(-c3cccc4c5c(ccc6c7ccccc7n(-c7ccccc7)c65)n(-c5ccc6c(c5)c5c(ccc7c8ccccc8n(-c8ccccc8)c75)n6-c5ccccc5)c34)nc(-c3ccccc3)n2)cc1] 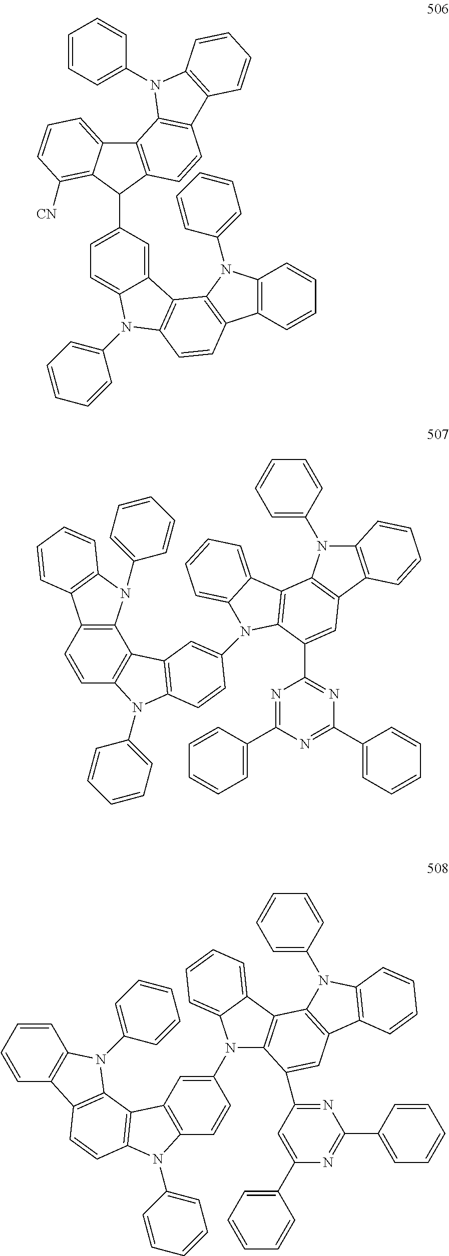 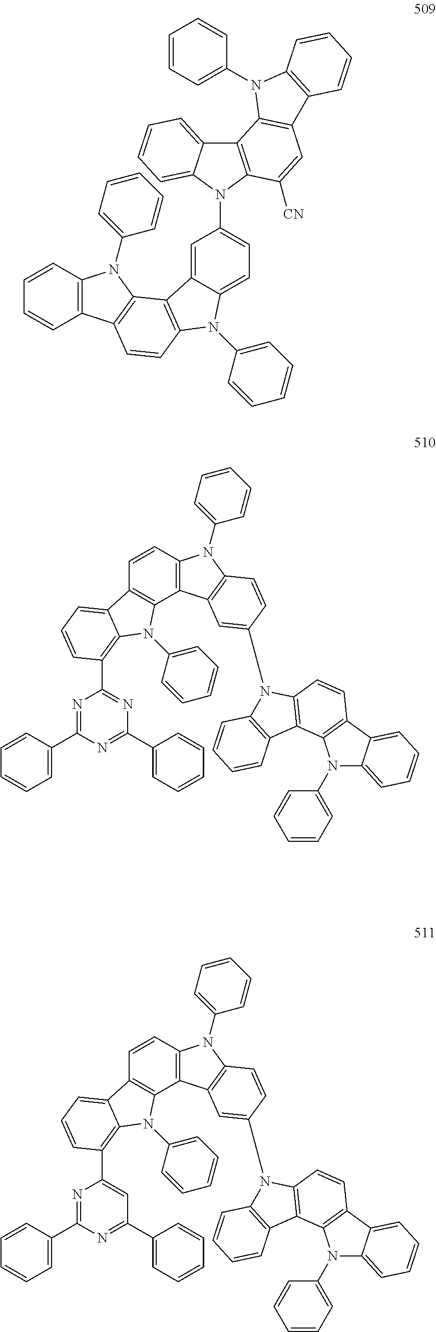 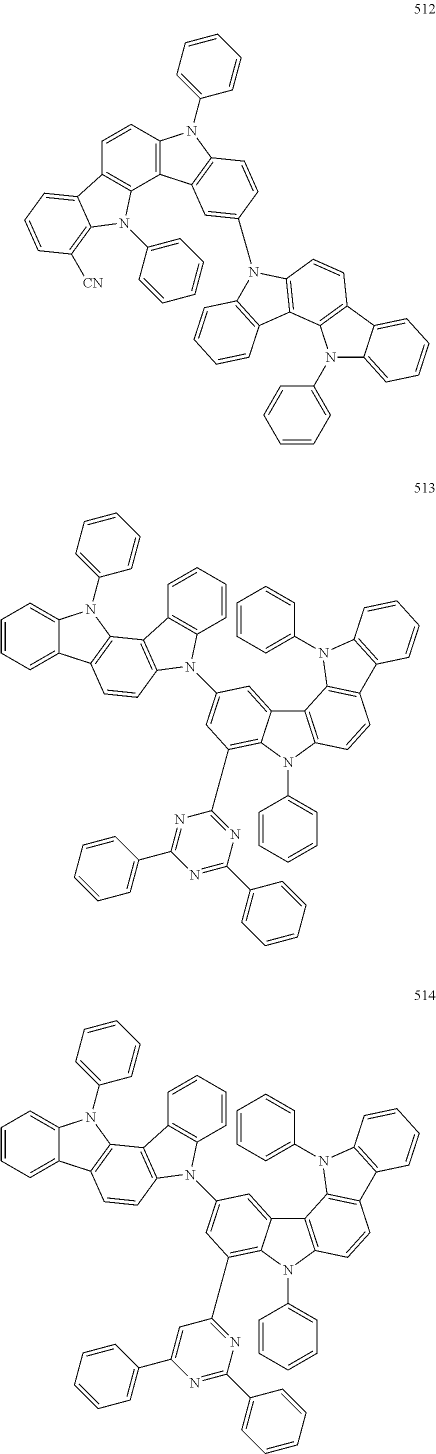 [SMILES: N#Cc1cccc2c3ccc4c(c5cc(-n6c7ccccc7c7c6ccc6c8ccccc8n(-c8ccccc8)c67)ccc5n4-c4ccccc4)c3n(-c3ccccc3)c12 ; c1ccc(-c2nc(-c3ccccc3)nc(-c3cc(-n4c5ccccc5c5c4ccc4c6ccccc6n(-c6ccccc6)c45)cc4c5c(ccc6c7ccccc7n(-c7ccccc7)c65)n(-c5ccccc5)c34)n2)cc1 ; c1ccc(-c2cc(-c3cc(-n4c5ccccc5c5c4ccc4c6ccccc6n(-c6ccccc6)c45)cc4c5c(ccc6c7ccccc7n(-c7ccccc7)c65)n(-c5ccccc5)c34)nc(-c3ccccc3)n2)cc1] 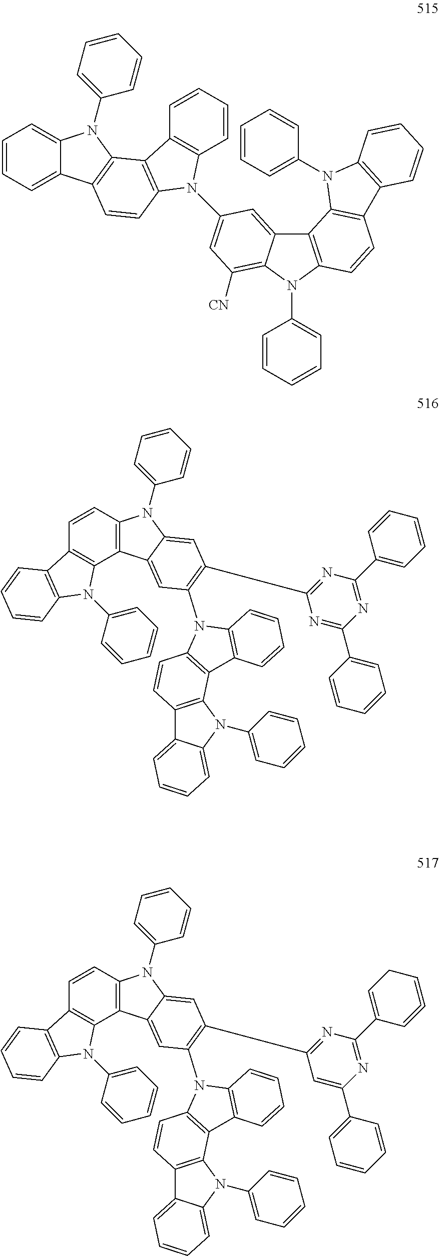 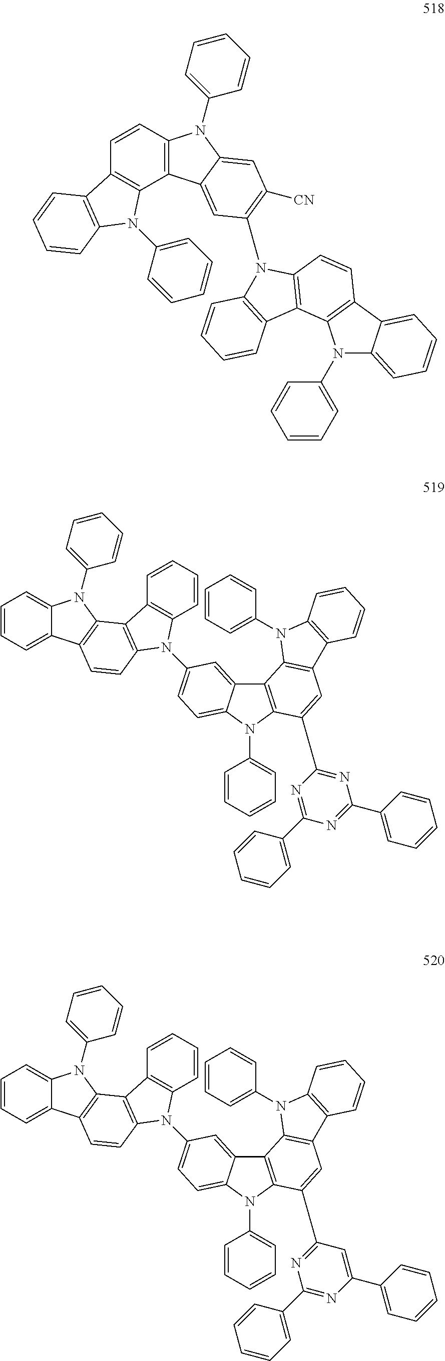 [SMILES: N#Cc1cc2c(cc1-n1c3ccccc3c3c1ccc1c4ccccc4n(-c4ccccc4)c13)c1c(ccc3c4ccccc4n(-c4ccccc4)c31)n2-c1ccccc1 ; c1ccc(-c2nc(-c3ccccc3)nc(-c3cc4c5ccccc5n(-c5ccccc5)c4c4c5cc(-n6c7ccccc7c7c6ccc6c8ccccc8n(-c8ccccc8)c67)ccc5n(-c5ccccc5)c34)n2)cc1 ; c1ccc(-c2cc(-c3cc4c5ccccc5n(-c5ccccc5)c4c4c5cc(-n6c7ccccc7c7c6ccc6c8ccccc8n(-c8ccccc8)c67)ccc5n(-c5ccccc5)c34)nc(-c3ccccc3)n2)cc1] 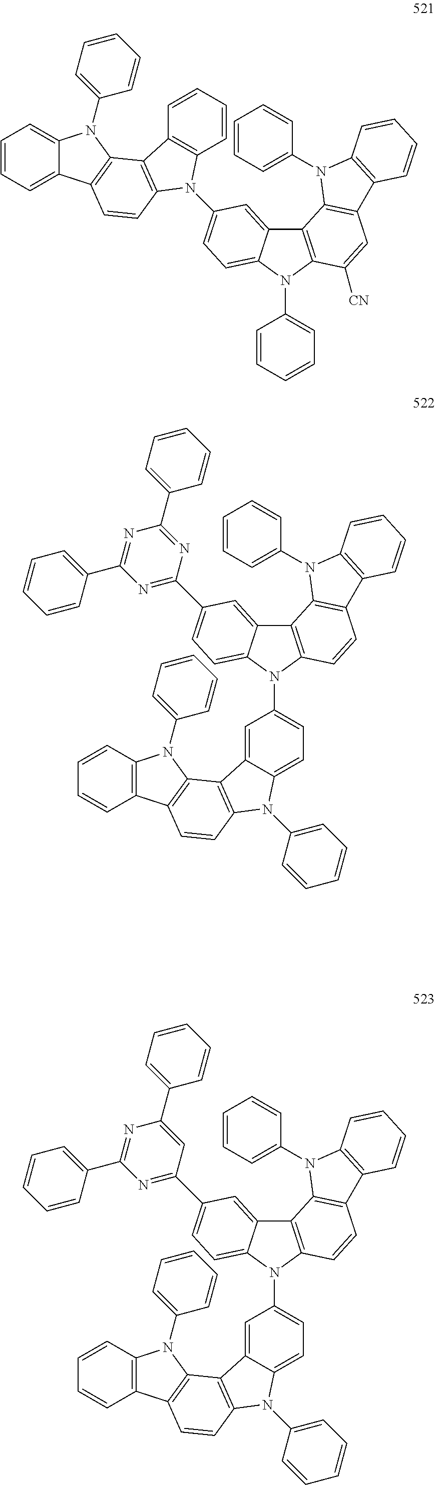 [SMILES: N#Cc1cc2c3ccccc3n(-c3ccccc3)c2c2c3cc(-n4c5ccccc5c5c4ccc4c6ccccc6n(-c6ccccc6)c45)ccc3n(-c3ccccc3)c12 ; c1ccc(-c2nc(-c3ccccc3)nc(-c3ccc4c(c3)c3c(ccc5c6ccccc6n(-c6ccccc6)c53)n4-c3ccc4c(c3)c3c(ccc5c6ccccc6n(-c6ccccc6)c53)n4-c3ccccc3)n2)cc1 ; c1ccc(-c2cc(-c3ccc4c(c3)c3c(ccc5c6ccccc6n(-c6ccccc6)c53)n4-c3ccc4c(c3)c3c(ccc5c6ccccc6n(-c6ccccc6)c53)n4-c3ccccc3)nc(-c3ccccc3)n2)cc1] 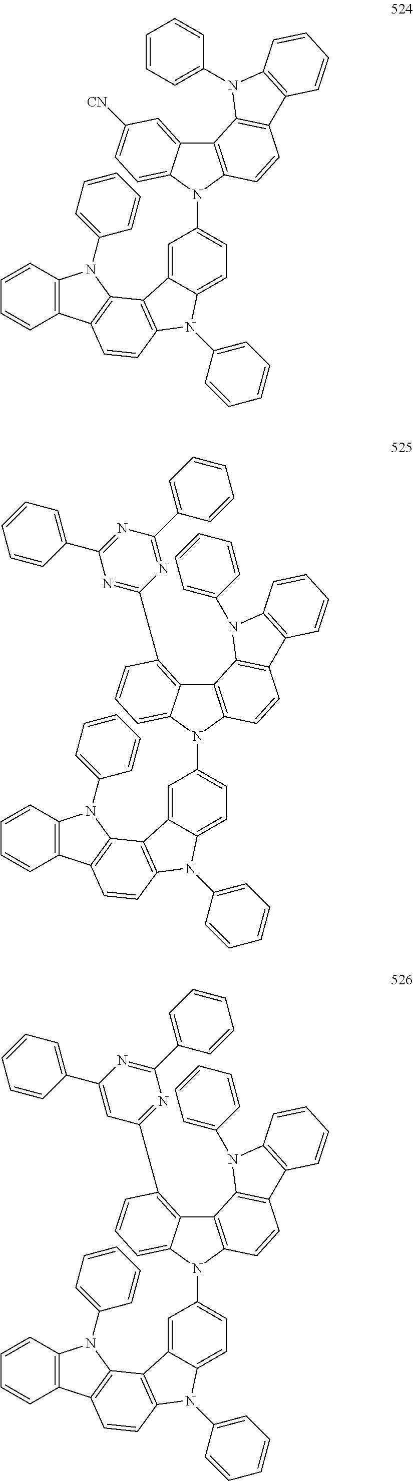 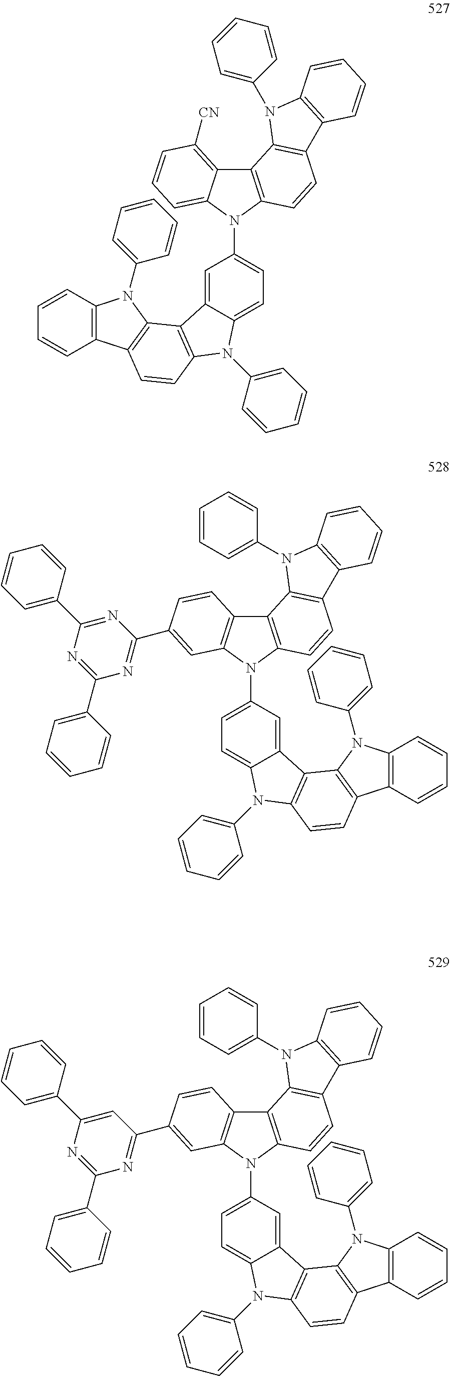 [SMILES: N#Cc1cccc2c1c1c(ccc3c4ccccc4n(-c4ccccc4)c31)n2-c1ccc2c(c1)c1c(ccc3c4ccccc4n(-c4ccccc4)c31)n2-c1ccccc1 ; c1ccc(-c2nc(-c3ccccc3)nc(-c3ccc4c5c(ccc6c7ccccc7n(-c7ccccc7)c65)n(-c5ccc6c(c5)c5c(ccc7c8ccccc8n(-c8ccccc8)c75)n6-c5ccccc5)c4c3)n2)cc1 ; c1ccc(-c2cc(-c3ccc4c5c(ccc6c7ccccc7n(-c7ccccc7)c65)n(-c5ccc6c(c5)c5c(ccc7c8ccccc8n(-c8ccccc8)c75)n6-c5ccccc5)c4c3)nc(-c3ccccc3)n2)cc1] 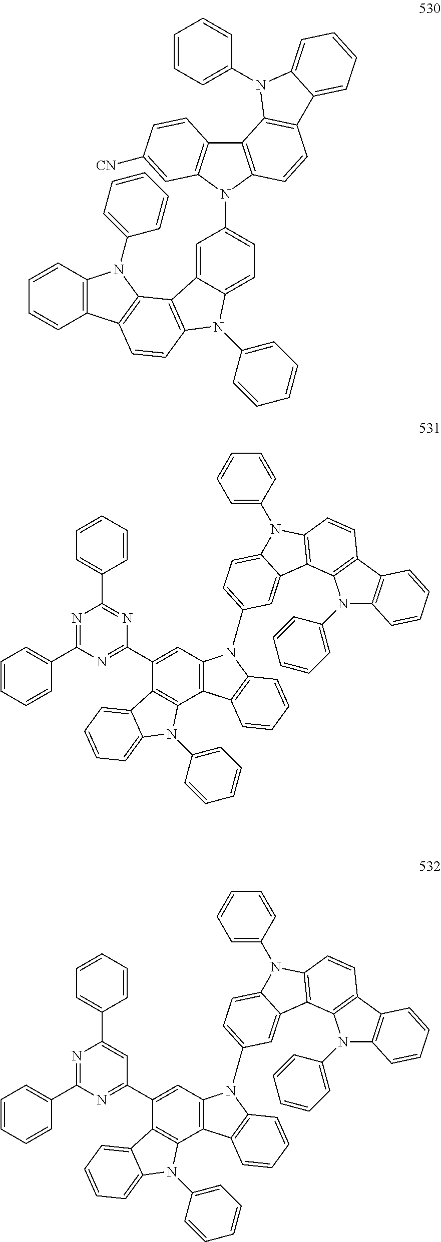 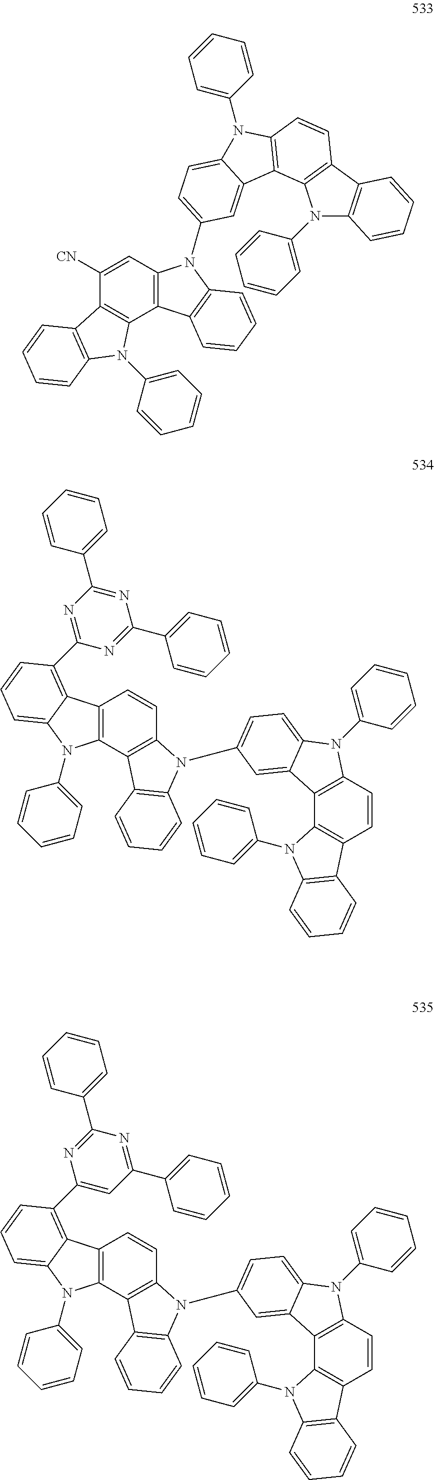 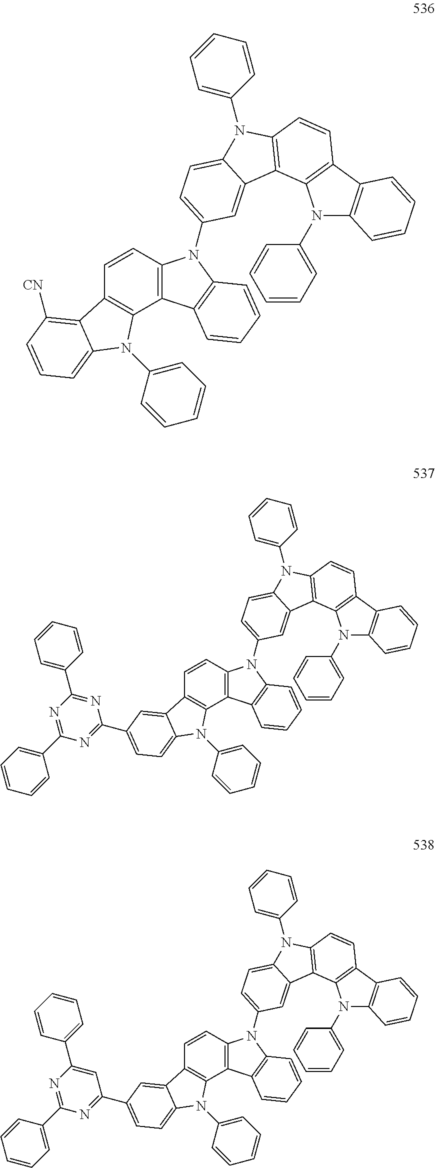 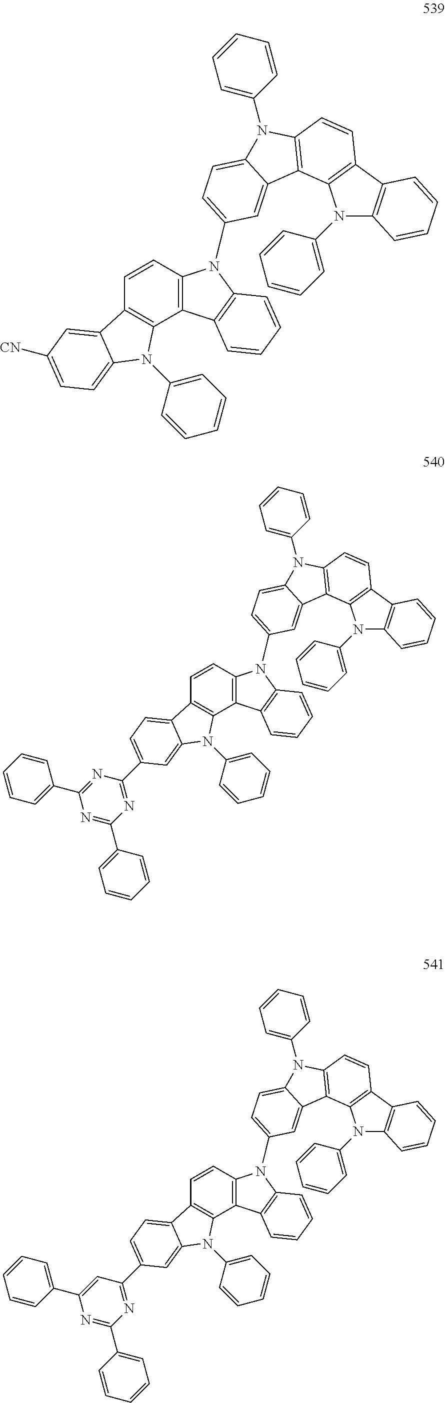 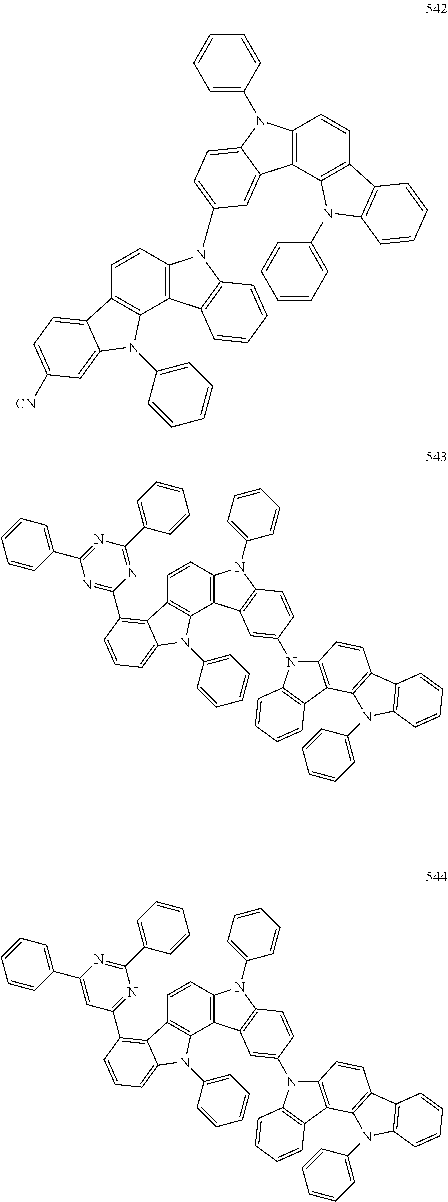 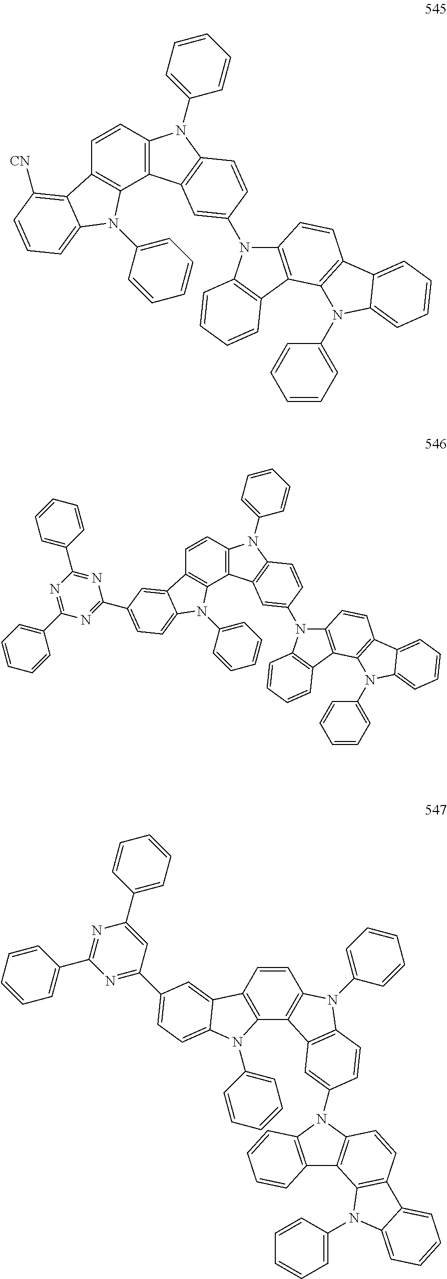 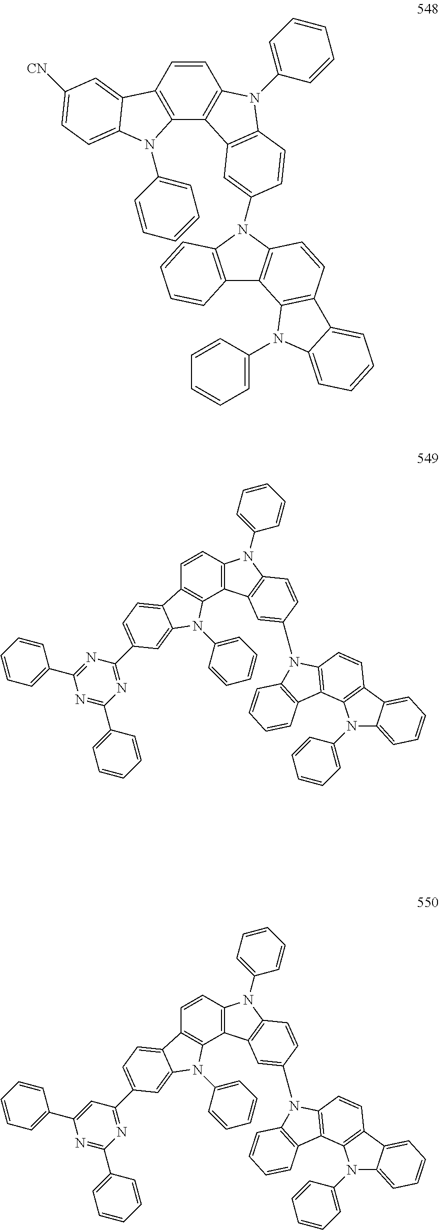 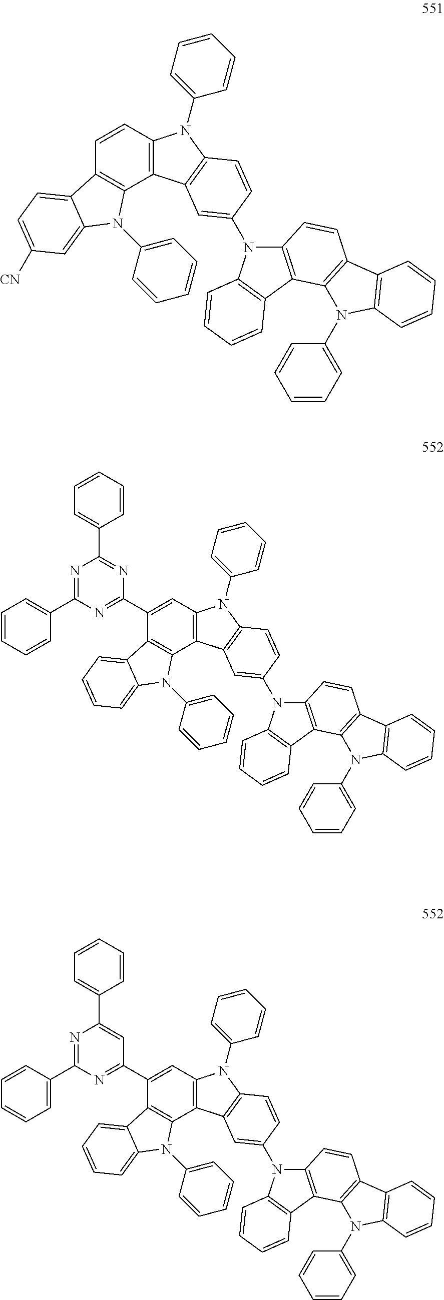 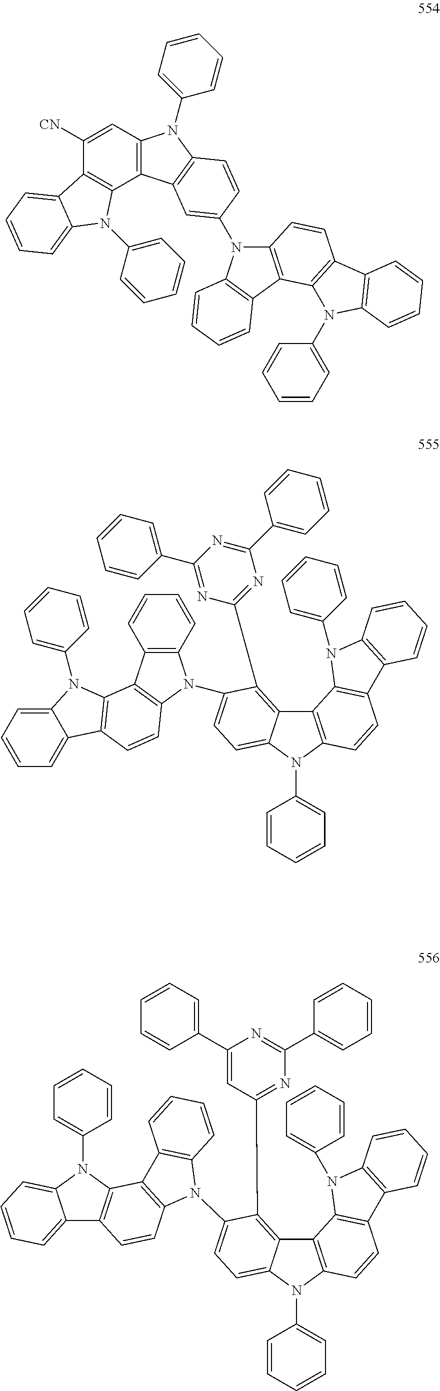 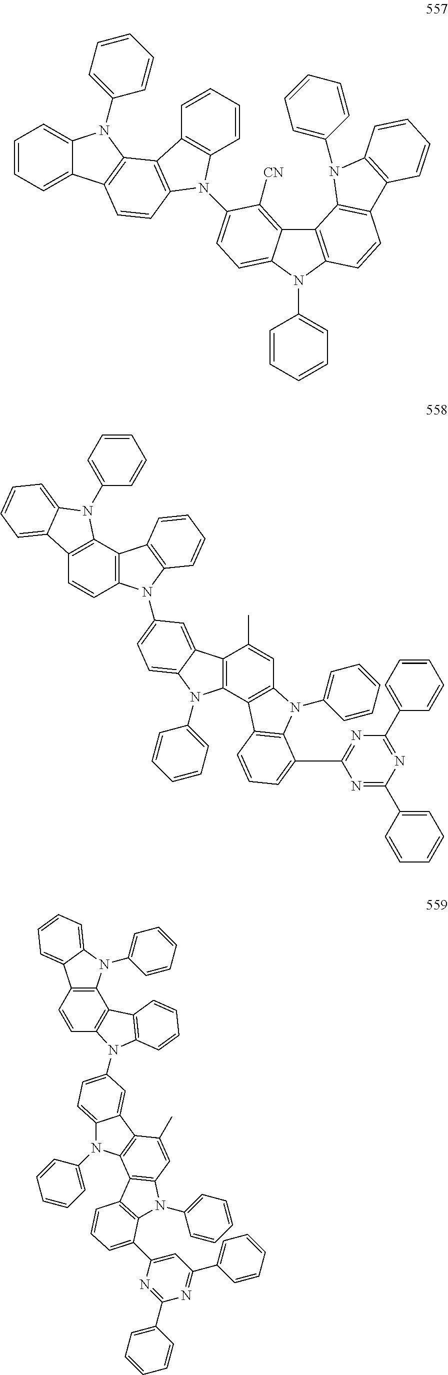 [SMILES: N#Cc1c(-n2c3ccccc3c3c2ccc2c4ccccc4n(-c4ccccc4)c23)ccc2c1c1c(ccc3c4ccccc4n(-c4ccccc4)c31)n2-c1ccccc1 ; Cc1cc2c(c3cccc(-c4nc(-c5ccccc5)nc(-c5ccccc5)n4)c3n2-c2ccccc2)c2c1c1cc(-n3c4ccccc4c4c3ccc3c5ccccc5n(-c5ccccc5)c34)ccc1n2-c1ccccc1 ; Cc1cc2c(c3cccc(-c4cc(-c5ccccc5)nc(-c5ccccc5)n4)c3n2-c2ccccc2)c2c1c1cc(-n3c4ccccc4c4c3ccc3c5ccccc5n(-c5ccccc5)c34)ccc1n2-c1ccccc1] 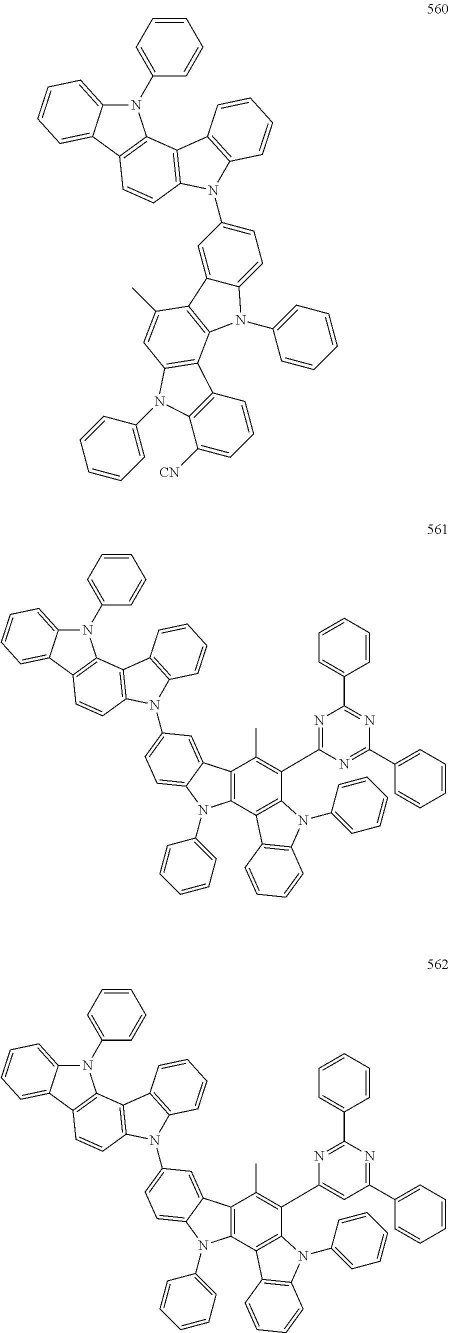 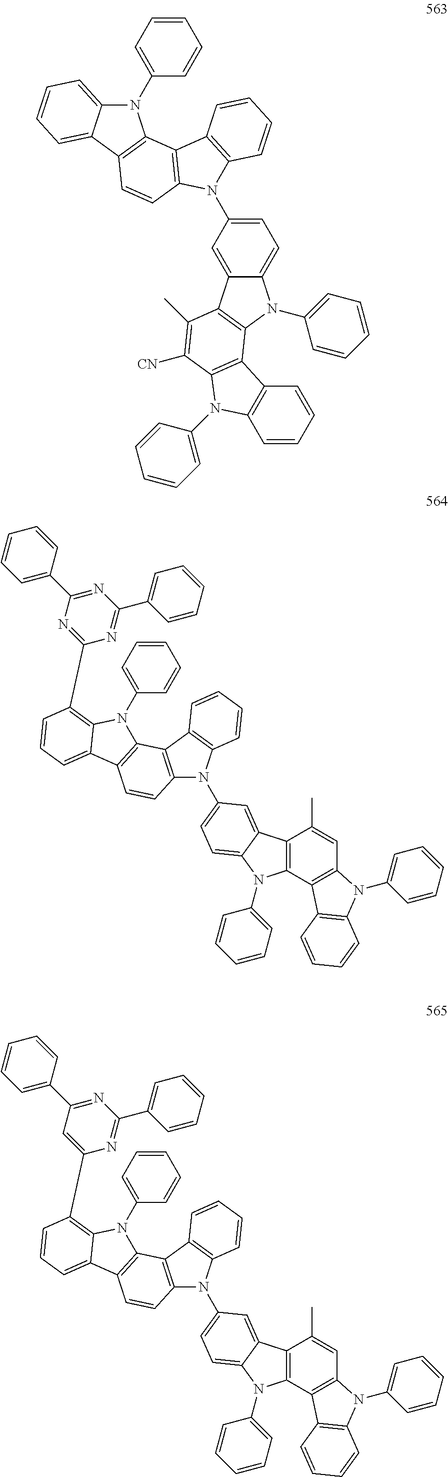 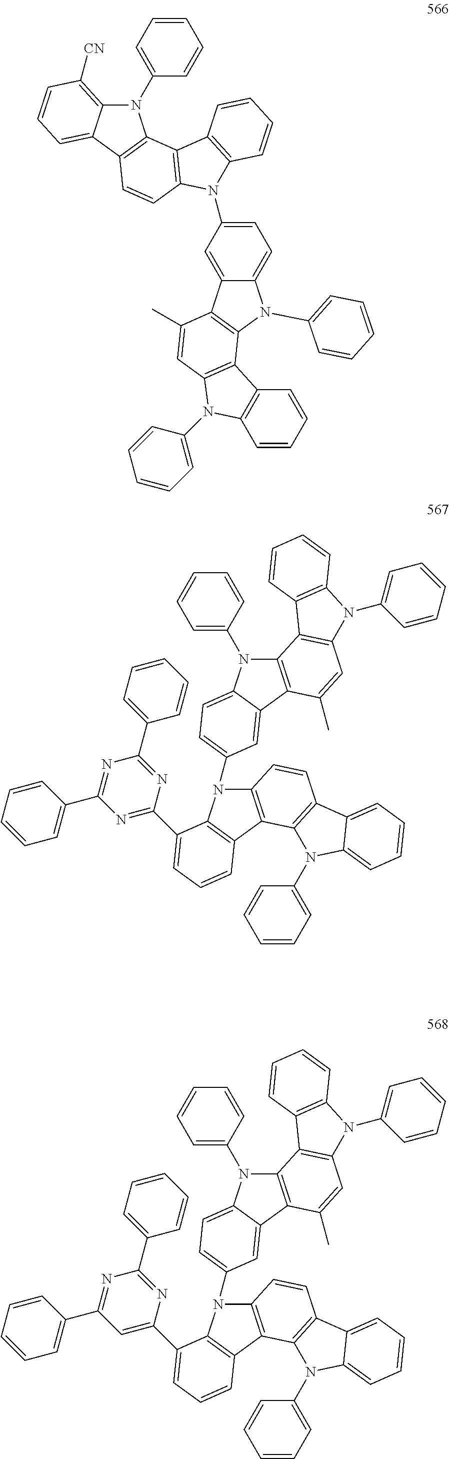 [SMILES: Cc1cc2c(c3ccccc3n2-c2ccccc2)c2c1c1cc(-n3c4ccccc4c4c3ccc3c5cccc(C#N)c5n(-c5ccccc5)c34)ccc1n2-c1ccccc1 ; Cc1cc2c(c3ccccc3n2-c2ccccc2)c2c1c1cc(-n3c4ccc5c6ccccc6n(-c6ccccc6)c5c4c4cccc(-c5nc(-c6ccccc6)nc(-c6ccccc6)n5)c43)ccc1n2-c1ccccc1 ; Cc1cc2c(c3ccccc3n2-c2ccccc2)c2c1c1cc(-n3c4ccc5c6ccccc6n(-c6ccccc6)c5c4c4cccc(-c5cc(-c6ccccc6)nc(-c6ccccc6)n5)c43)ccc1n2-c1ccccc1] 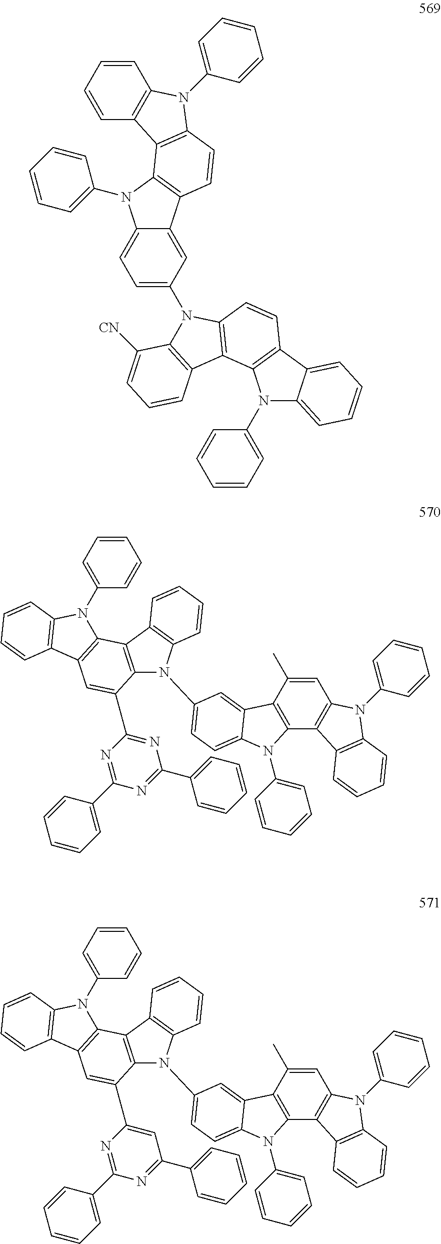 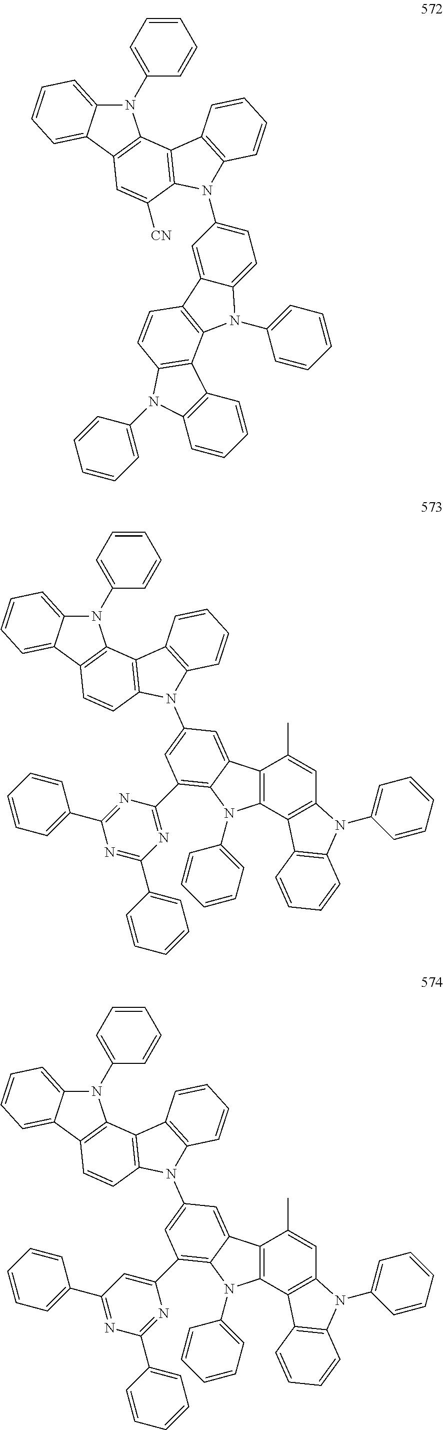 [SMILES: N#Cc1cc2c3ccccc3n(-c3ccccc3)c2c2c3ccccc3n(-c3ccc4c(c3)c3ccc5c(c6ccccc6n5-c5ccccc5)c3n4-c3ccccc3)c12 ; Cc1cc2c(c3ccccc3n2-c2ccccc2)c2c1c1cc(-n3c4ccccc4c4c3ccc3c5ccccc5n(-c5ccccc5)c34)cc(-c3nc(-c4ccccc4)nc(-c4ccccc4)n3)c1n2-c1ccccc1 ; Cc1cc2c(c3ccccc3n2-c2ccccc2)c2c1c1cc(-n3c4ccccc4c4c3ccc3c5ccccc5n(-c5ccccc5)c34)cc(-c3cc(-c4ccccc4)nc(-c4ccccc4)n3)c1n2-c1ccccc1] 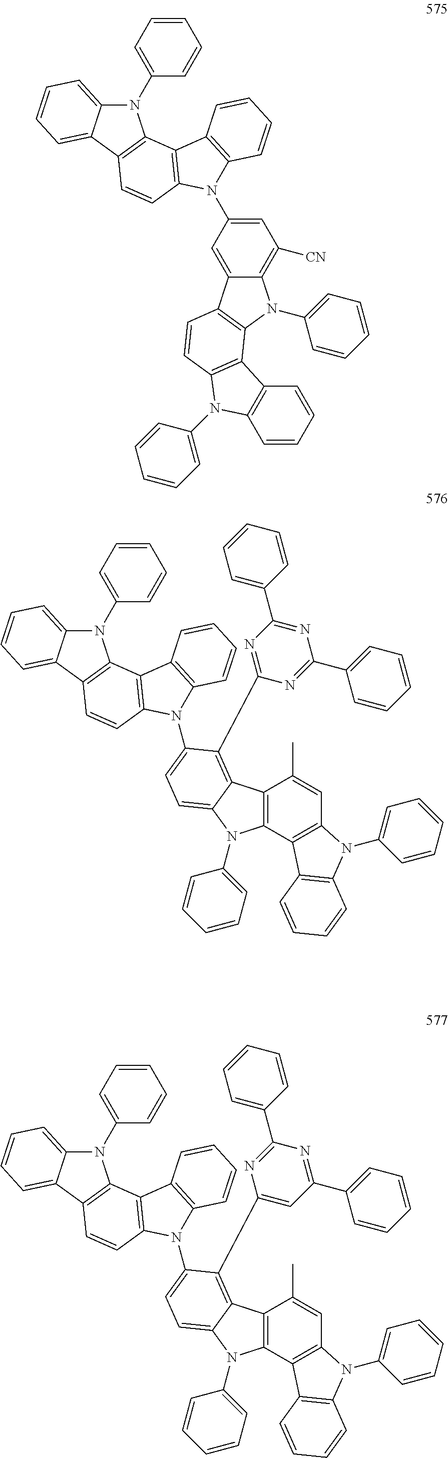 [SMILES: N#Cc1cc(-n2c3ccccc3c3c2ccc2c4ccccc4n(-c4ccccc4)c23)cc2c3ccc4c(c5ccccc5n4-c4ccccc4)c3n(-c3ccccc3)c12 ; Cc1cc2c(c3ccccc3n2-c2ccccc2)c2c1c1c(-c3nc(-c4ccccc4)nc(-c4ccccc4)n3)c(-n3c4ccccc4c4c3ccc3c5ccccc5n(-c5ccccc5)c34)ccc1n2-c1ccccc1 ; Cc1cc2c(c3ccccc3n2-c2ccccc2)c2c1c1c(-c3cc(-c4ccccc4)nc(-c4ccccc4)n3)c(-n3c4ccccc4c4c3ccc3c5ccccc5n(-c5ccccc5)c34)ccc1n2-c1ccccc1] 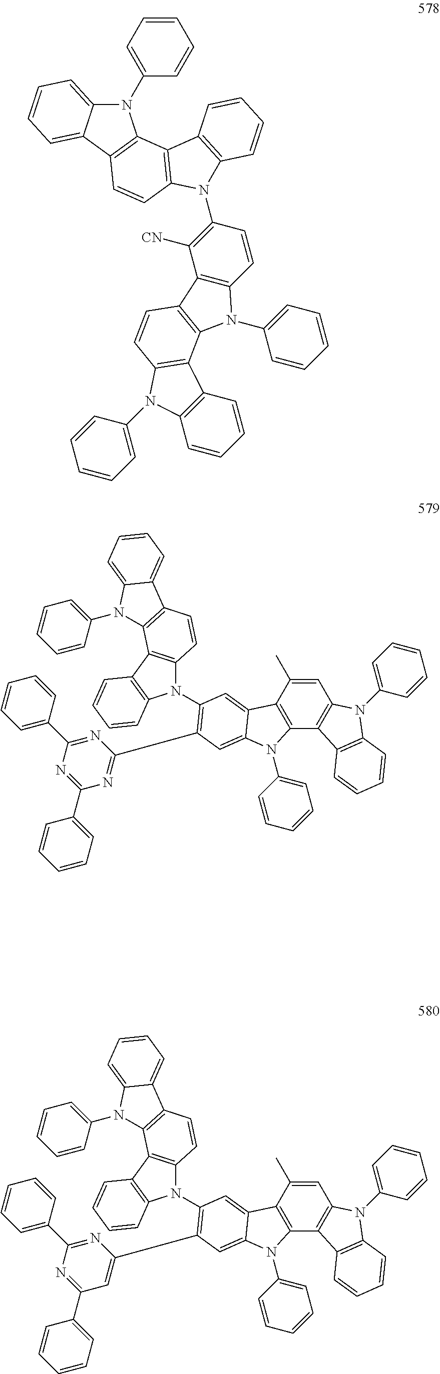 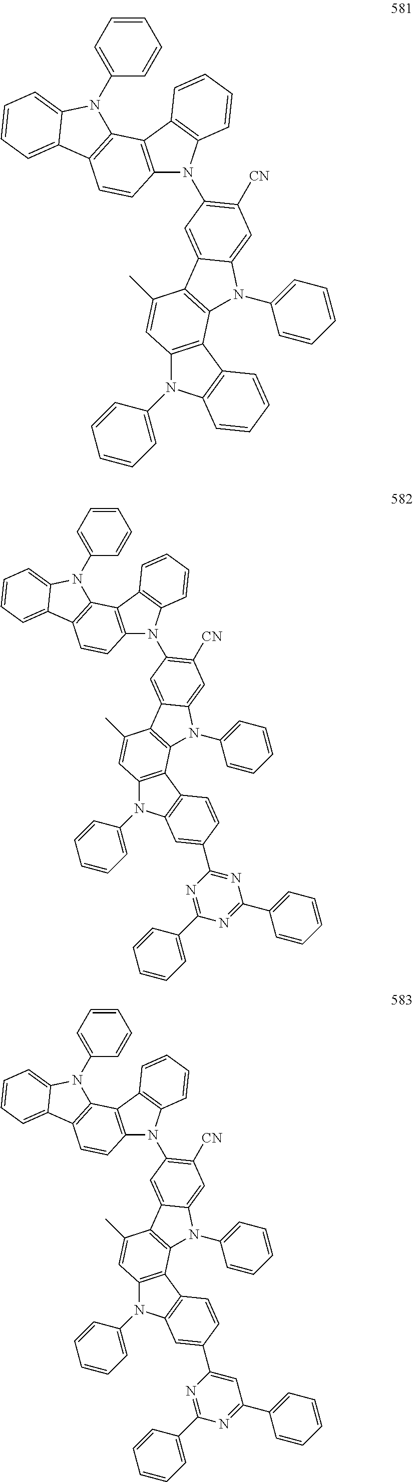 [SMILES: Cc1cc2c(c3ccccc3n2-c2ccccc2)c2c1c1cc(-n3c4ccccc4c4c3ccc3c5ccccc5n(-c5ccccc5)c34)c(C#N)cc1n2-c1ccccc1 ; Cc1cc2c(c3ccc(-c4nc(-c5ccccc5)nc(-c5ccccc5)n4)cc3n2-c2ccccc2)c2c1c1cc(-n3c4ccccc4c4c3ccc3c5ccccc5n(-c5ccccc5)c34)c(C#N)cc1n2-c1ccccc1 ; Cc1cc2c(c3ccc(-c4cc(-c5ccccc5)nc(-c5ccccc5)n4)cc3n2-c2ccccc2)c2c1c1cc(-n3c4ccccc4c4c3ccc3c5ccccc5n(-c5ccccc5)c34)c(C#N)cc1n2-c1ccccc1] 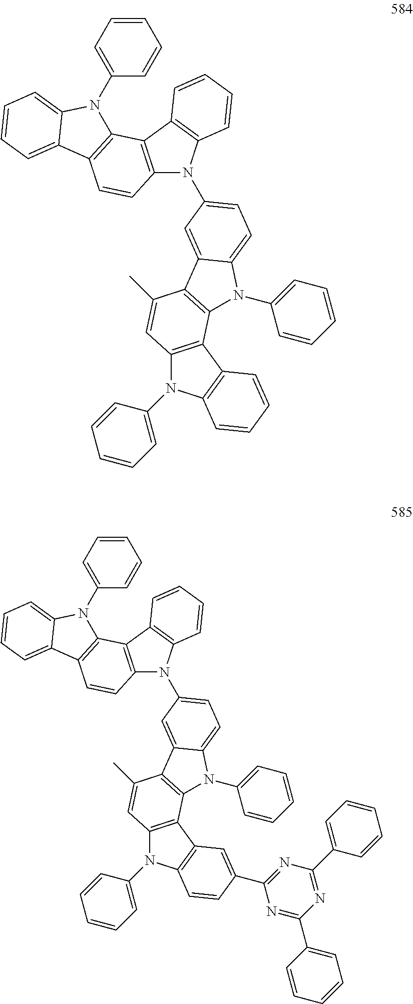 [SMILES: Cc1cc2c(c3ccccc3n2-c2ccccc2)c2c1c1cc(-n3c4ccccc4c4c3ccc3c5ccccc5n(-c5ccccc5)c34)ccc1n2-c1ccccc1 ; Cc1cc2c(c3cc(-c4nc(-c5ccccc5)nc(-c5ccccc5)n4)ccc3n2-c2ccccc2)c2c1c1cc(-n3c4ccccc4c4c3ccc3c5ccccc5n(-c5ccccc5)c34)ccc1n2-c1ccccc1] 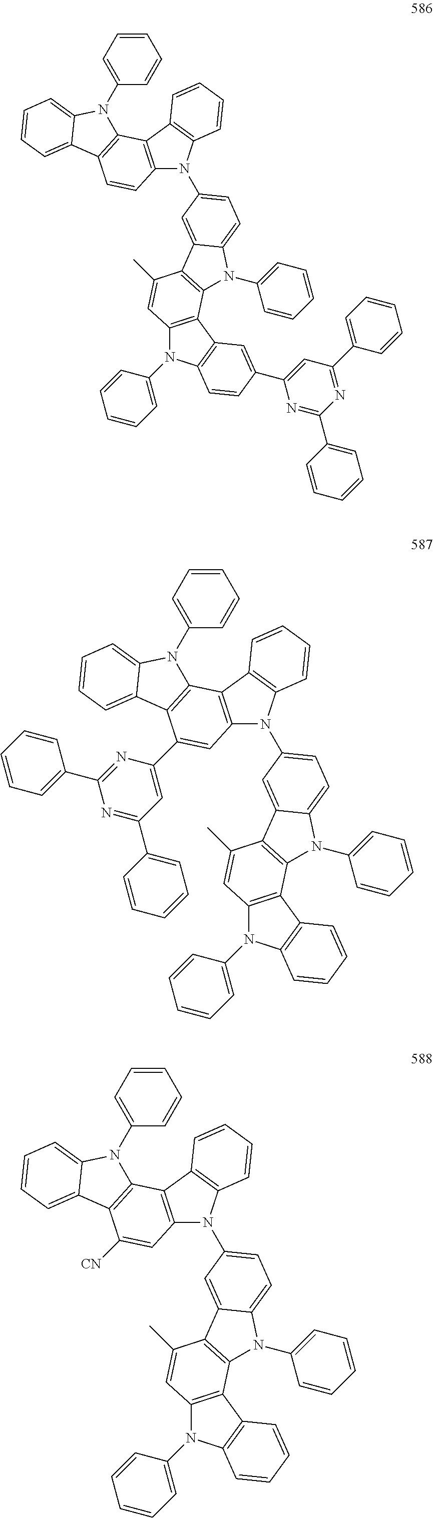 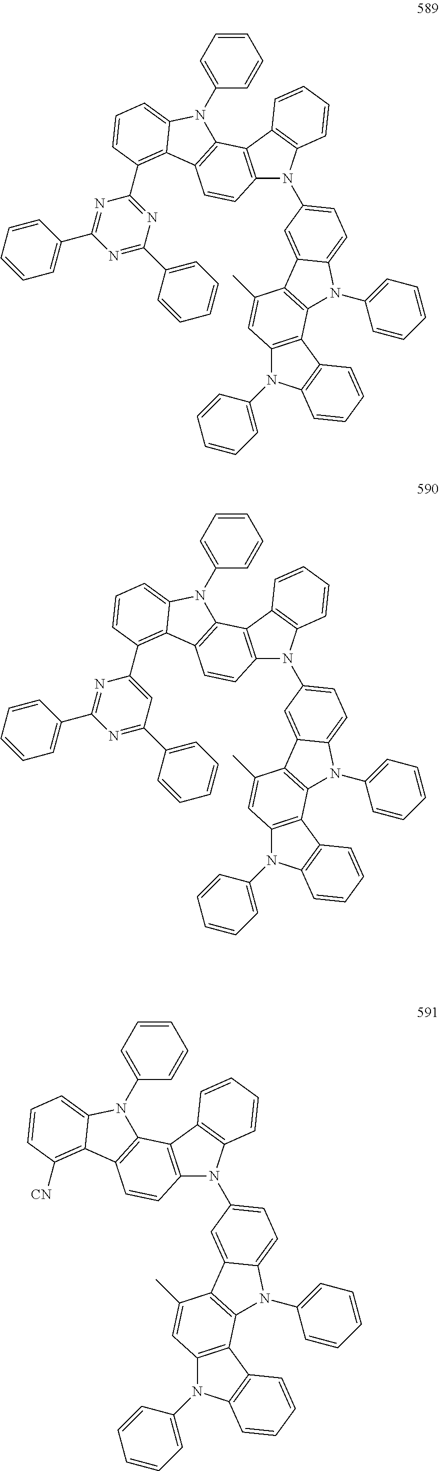 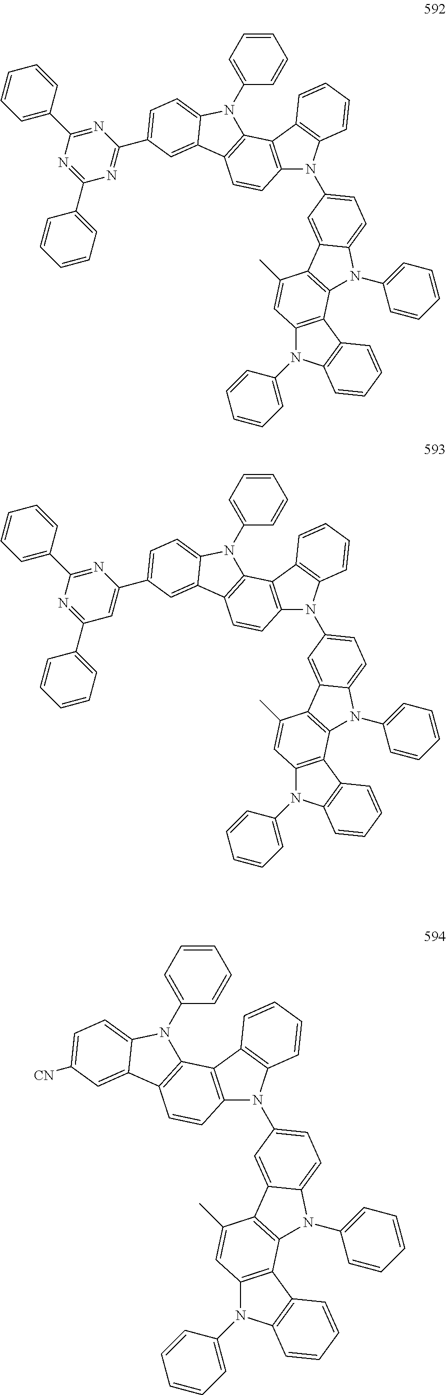 [SMILES: Cc1cc2c(c3ccccc3n2-c2ccccc2)c2c1c1cc(-n3c4ccccc4c4c3ccc3c5cc(-c6nc(-c7ccccc7)nc(-c7ccccc7)n6)ccc5n(-c5ccccc5)c34)ccc1n2-c1ccccc1 ; Cc1cc2c(c3ccccc3n2-c2ccccc2)c2c1c1cc(-n3c4ccccc4c4c3ccc3c5cc(-c6cc(-c7ccccc7)nc(-c7ccccc7)n6)ccc5n(-c5ccccc5)c34)ccc1n2-c1ccccc1 ; Cc1cc2c(c3ccccc3n2-c2ccccc2)c2c1c1cc(-n3c4ccccc4c4c3ccc3c5cc(C#N)ccc5n(-c5ccccc5)c34)ccc1n2-c1ccccc1] 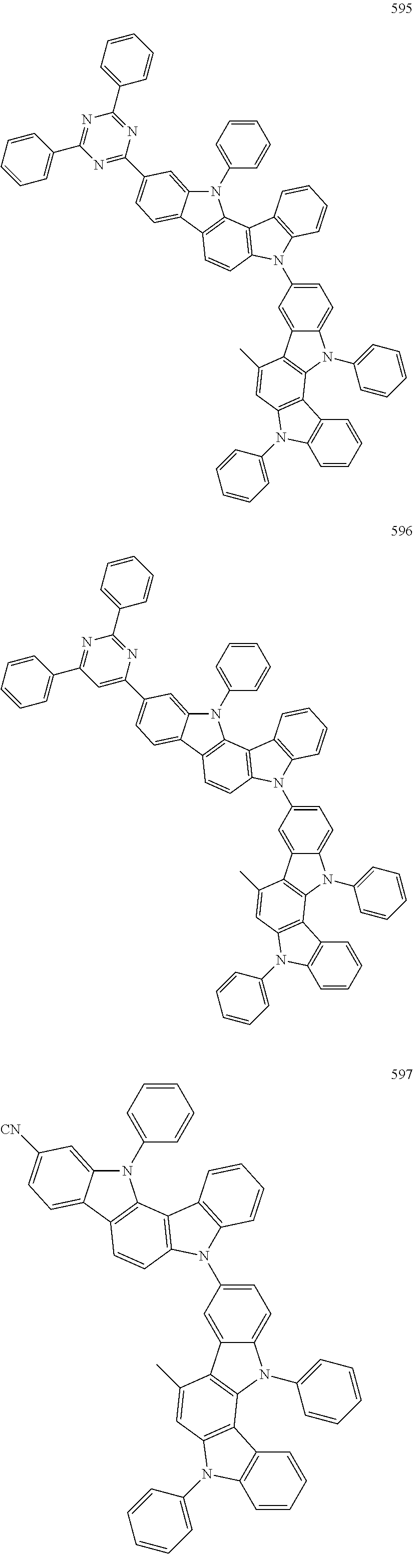 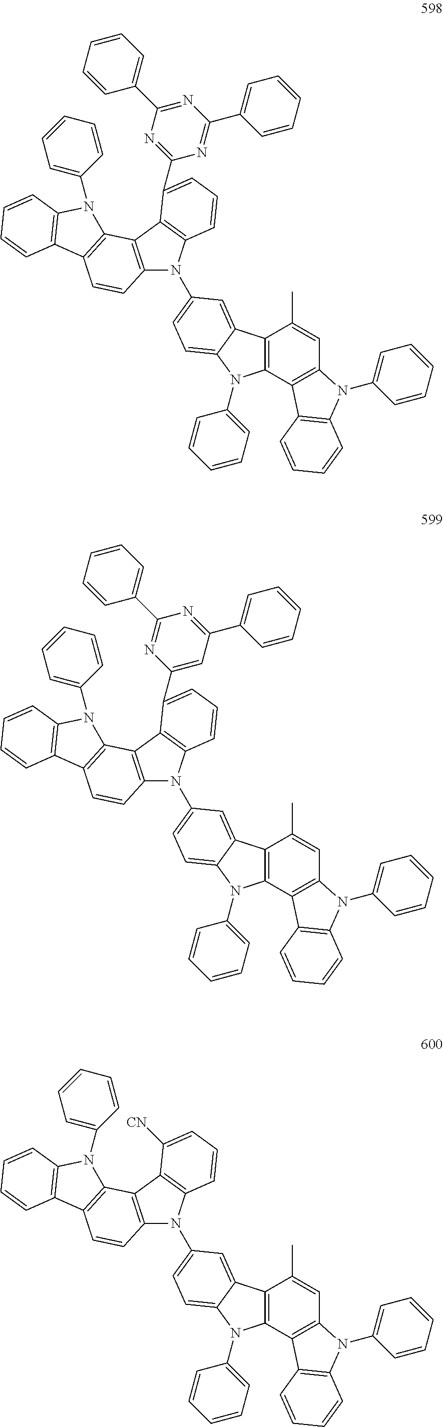 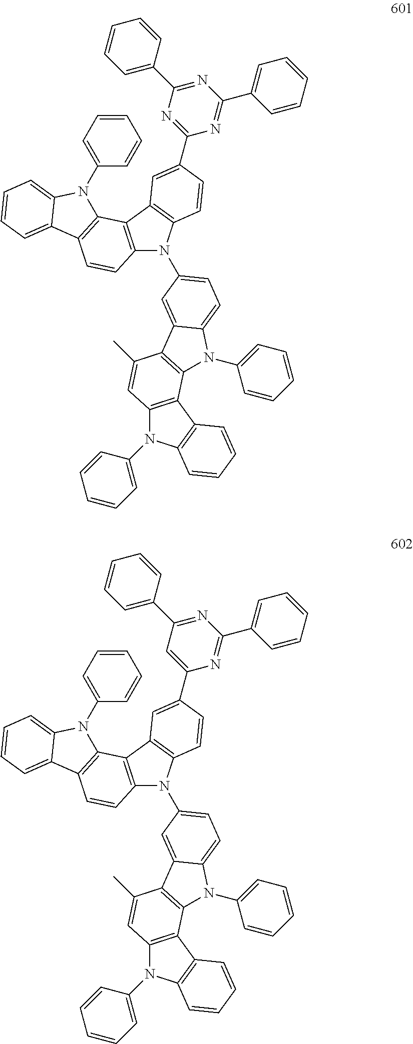 [SMILES: Cc1cc2c(c3ccccc3n2-c2ccccc2)c2c1c1cc(-n3c4ccc(-c5nc(-c6ccccc6)nc(-c6ccccc6)n5)cc4c4c3ccc3c5ccccc5n(-c5ccccc5)c34)ccc1n2-c1ccccc1 ; Cc1cc2c(c3ccccc3n2-c2ccccc2)c2c1c1cc(-n3c4ccc(-c5cc(-c6ccccc6)nc(-c6ccccc6)n5)cc4c4c3ccc3c5ccccc5n(-c5ccccc5)c34)ccc1n2-c1ccccc1] 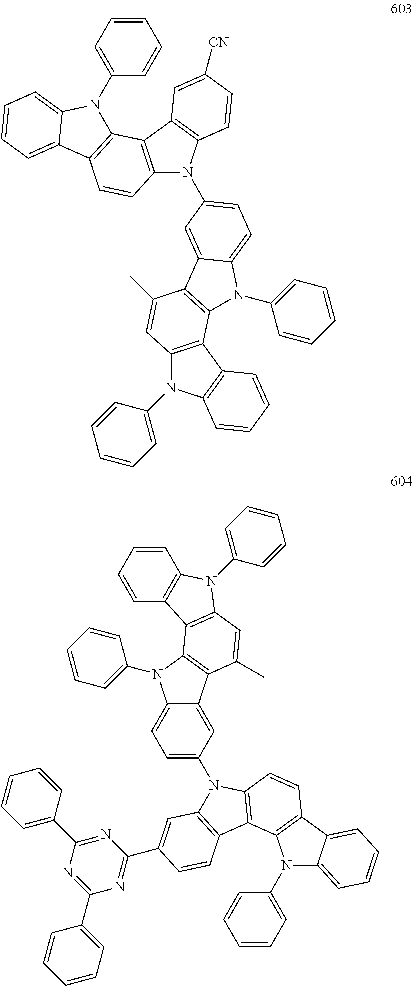 [SMILES: Cc1cc2c(c3ccccc3n2-c2ccccc2)c2c1c1cc(-n3c4ccc(C#N)cc4c4c3ccc3c5ccccc5n(-c5ccccc5)c34)ccc1n2-c1ccccc1 ; Cc1cc2c(c3ccccc3n2-c2ccccc2)c2c1c1cc(-n3c4cc(-c5nc(-c6ccccc6)nc(-c6ccccc6)n5)ccc4c4c3ccc3c5ccccc5n(-c5ccccc5)c34)ccc1n2-c1ccccc1] 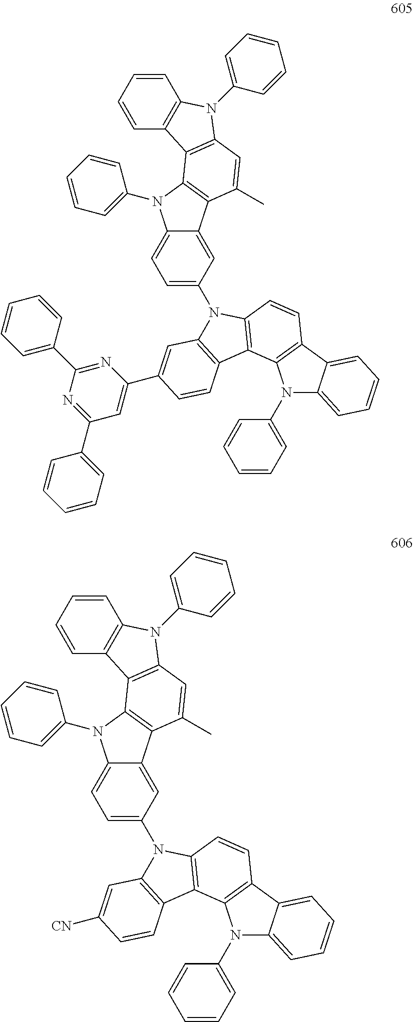 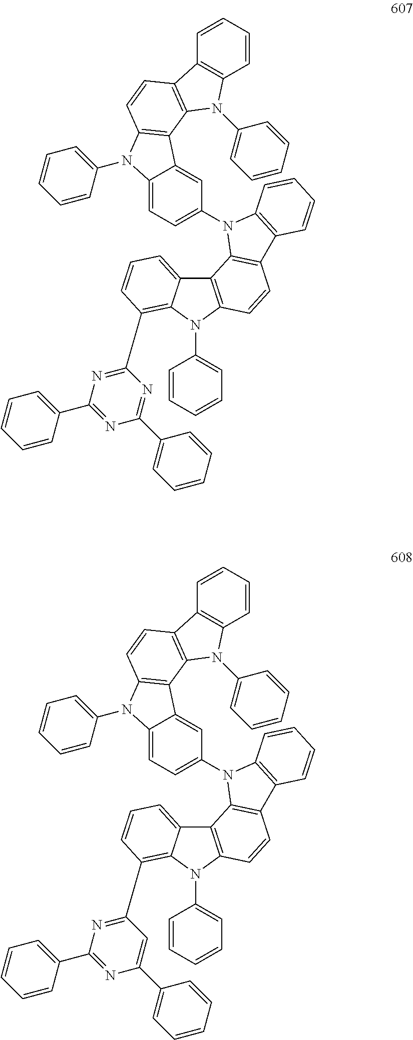 [SMILES: c1ccc(-c2nc(-c3ccccc3)nc(-c3cccc4c5c(ccc6c7ccccc7n(-c7ccc8c(c7)c7c(ccc9c%10ccccc%10n(-c%10ccccc%10)c97)n8-c7ccccc7)c65)n(-c5ccccc5)c34)n2)cc1 ; c1ccc(-c2cc(-c3cccc4c5c(ccc6c7ccccc7n(-c7ccc8c(c7)c7c(ccc9c%10ccccc%10n(-c%10ccccc%10)c97)n8-c7ccccc7)c65)n(-c5ccccc5)c34)nc(-c3ccccc3)n2)cc1] 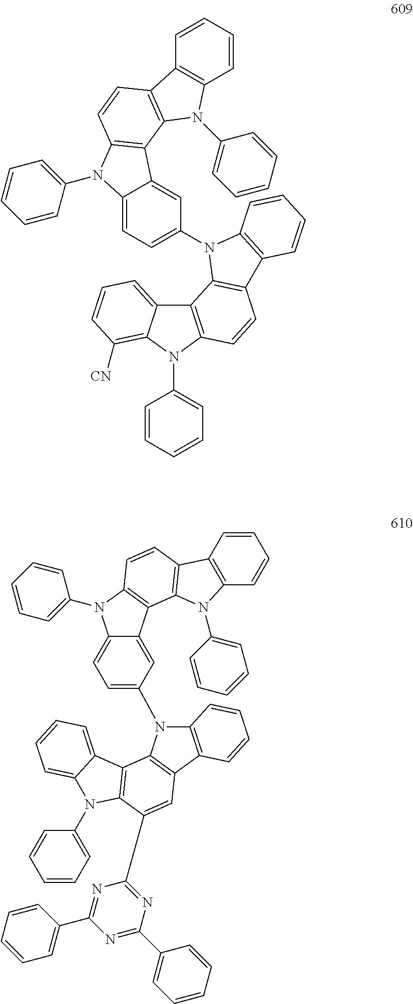 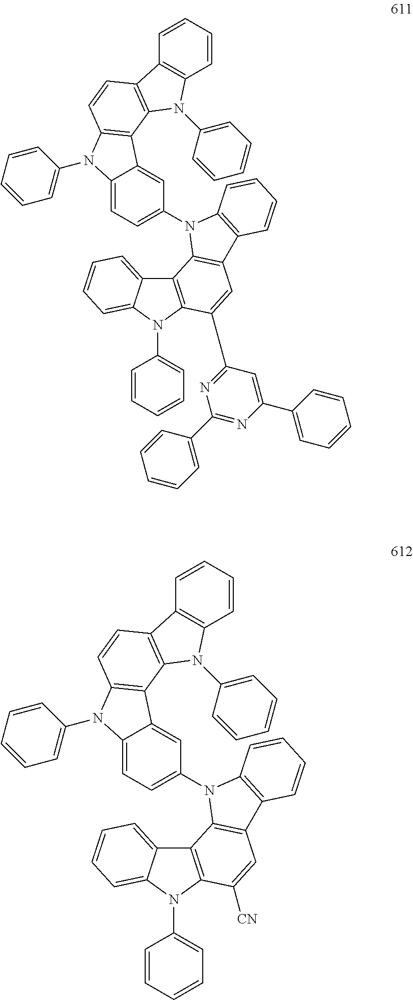 [SMILES: c1ccc(-c2cc(-c3cc4c5ccccc5n(-c5ccc6c(c5)c5c(ccc7c8ccccc8n(-c8ccccc8)c75)n6-c5ccccc5)c4c4c5ccccc5n(-c5ccccc5)c34)nc(-c3ccccc3)n2)cc1 ; N#Cc1cc2c3ccccc3n(-c3ccc4c(c3)c3c(ccc5c6ccccc6n(-c6ccccc6)c53)n4-c3ccccc3)c2c2c3ccccc3n(-c3ccccc3)c12] 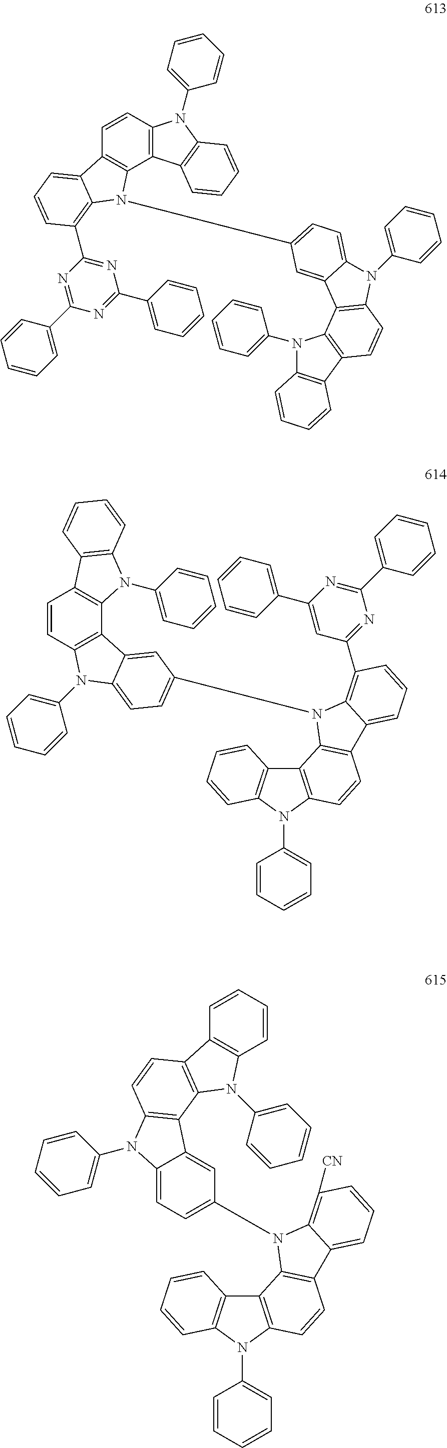 [SMILES: c1ccc(-c2nc(-c3ccccc3)nc(-c3cccc4c5ccc6c(c7ccccc7n6-c6ccccc6)c5n(-c5ccc6c(c5)c5c(ccc7c8ccccc8n(-c8ccccc8)c75)n6-c5ccccc5)c34)n2)cc1 ; c1ccc(-c2cc(-c3cccc4c5ccc6c(c7ccccc7n6-c6ccccc6)c5n(-c5ccc6c(c5)c5c(ccc7c8ccccc8n(-c8ccccc8)c75)n6-c5ccccc5)c34)nc(-c3ccccc3)n2)cc1 ; N#Cc1cccc2c3ccc4c(c5ccccc5n4-c4ccccc4)c3n(-c3ccc4c(c3)c3c(ccc5c6ccccc6n(-c6ccccc6)c53)n4-c3ccccc3)c12] 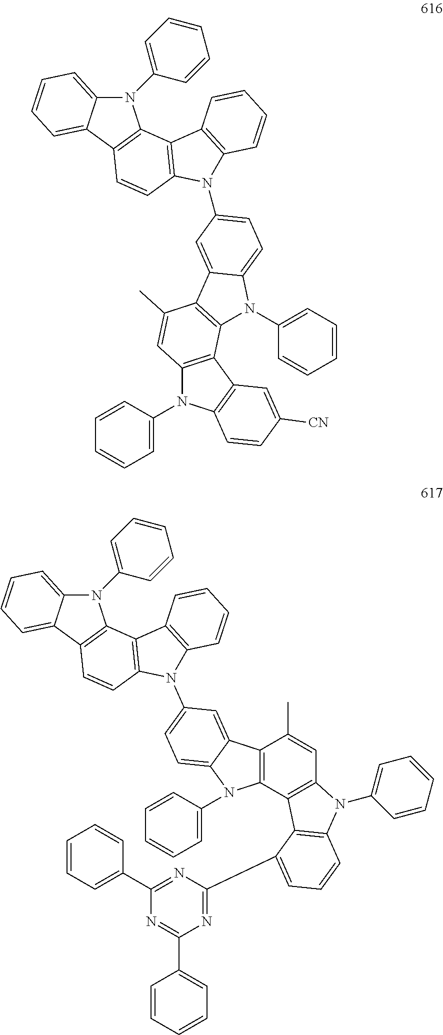 [SMILES: Cc1cc2c(c3cc(C#N)ccc3n2-c2ccccc2)c2c1c1cc(-n3c4ccccc4c4c3ccc3c5ccccc5n(-c5ccccc5)c34)ccc1n2-c1ccccc1 ; Cc1cc2c(c3c(-c4nc(-c5ccccc5)nc(-c5ccccc5)n4)cccc3n2-c2ccccc2)c2c1c1cc(-n3c4ccccc4c4c3ccc3c5ccccc5n(-c5ccccc5)c34)ccc1n2-c1ccccc1] 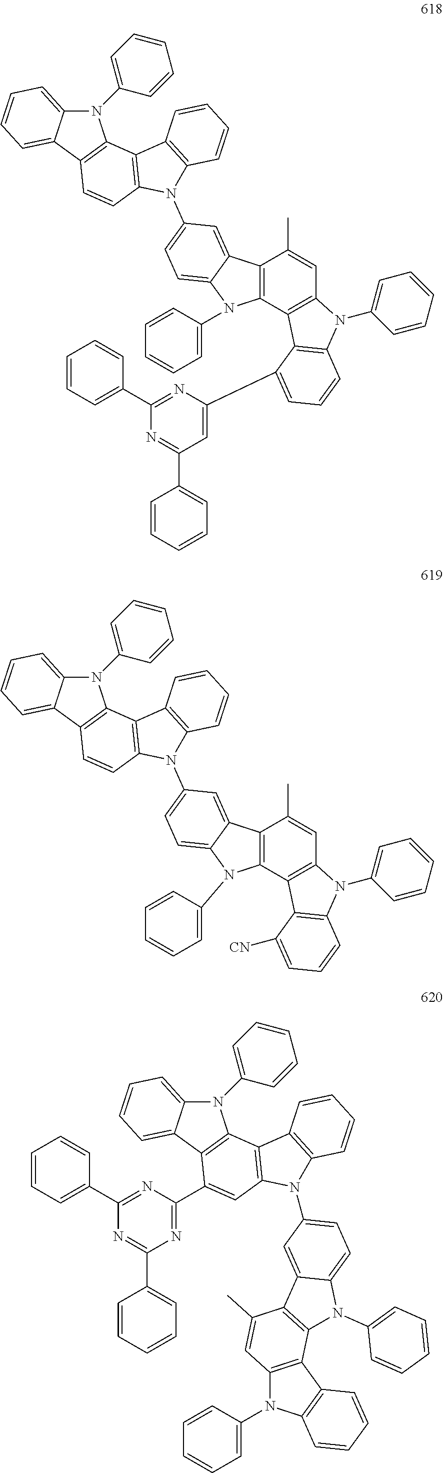 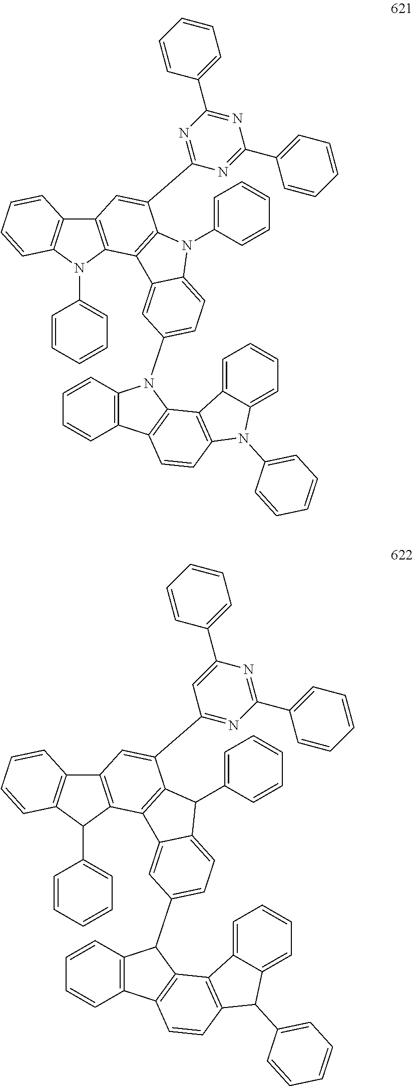 [SMILES: c1ccc(-c2nc(-c3ccccc3)nc(-c3cc4c5ccccc5n(-c5ccccc5)c4c4c5cc(-n6c7ccccc7c7ccc8c(c9ccccc9n8-c8ccccc8)c76)ccc5n(-c5ccccc5)c34)n2)cc1 ; c1ccc(-c2cc(-c3cc4c(c5c3C(c3ccccc3)c3ccc(C6c7ccccc7-c7ccc8c(c76)-c6ccccc6C8c6ccccc6)cc3-5)C(c3ccccc3)c3ccccc3-4)nc(-c3ccccc3)n2)cc1] 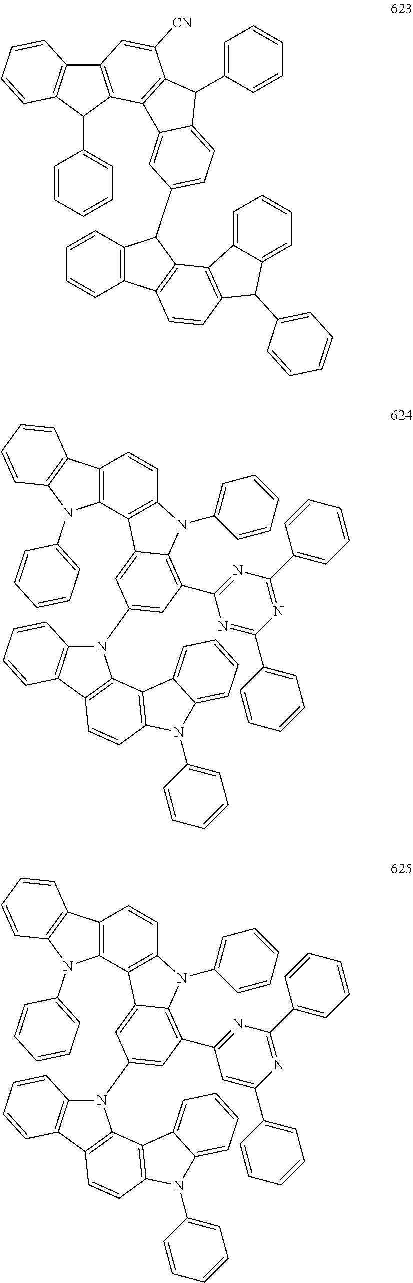 [SMILES: N#Cc1cc2c(c3c1C(c1ccccc1)c1ccc(C4c5ccccc5-c5ccc6c(c54)-c4ccccc4C6c4ccccc4)cc1-3)C(c1ccccc1)c1ccccc1-2 ; c1ccc(-c2nc(-c3ccccc3)nc(-c3cc(-n4c5ccccc5c5ccc6c(c7ccccc7n6-c6ccccc6)c54)cc4c5c(ccc6c7ccccc7n(-c7ccccc7)c65)n(-c5ccccc5)c34)n2)cc1 ; c1ccc(-c2cc(-c3cc(-n4c5ccccc5c5ccc6c(c7ccccc7n6-c6ccccc6)c54)cc4c5c(ccc6c7ccccc7n(-c7ccccc7)c65)n(-c5ccccc5)c34)nc(-c3ccccc3)n2)cc1] 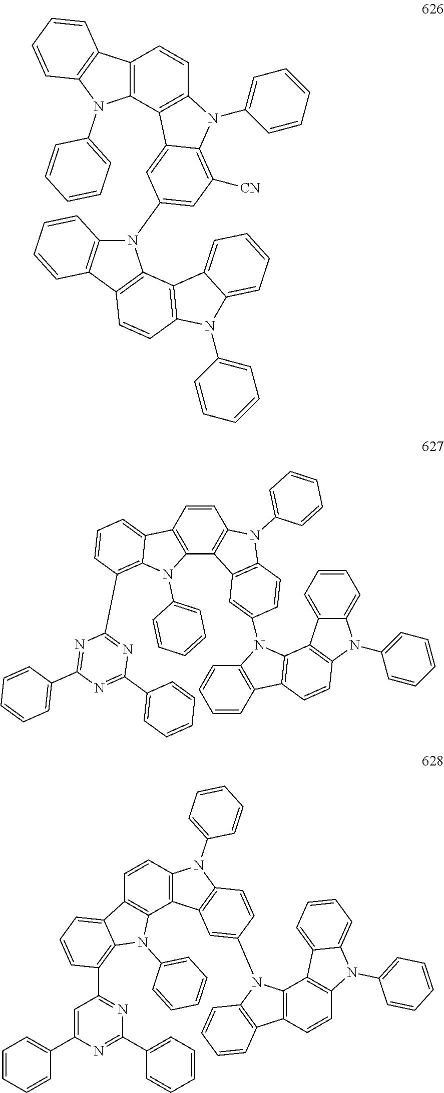 [SMILES: N#Cc1cc(-n2c3ccccc3c3ccc4c(c5ccccc5n4-c4ccccc4)c32)cc2c3c(ccc4c5ccccc5n(-c5ccccc5)c43)n(-c3ccccc3)c12 ; c1ccc(-c2nc(-c3ccccc3)nc(-c3cccc4c5ccc6c(c7cc(-n8c9ccccc9c9ccc%10c(c%11ccccc%11n%10-c%10ccccc%10)c98)ccc7n6-c6ccccc6)c5n(-c5ccccc5)c34)n2)cc1 ; c1ccc(-c2cc(-c3cccc4c5ccc6c(c7cc(-n8c9ccccc9c9ccc%10c(c%11ccccc%11n%10-c%10ccccc%10)c98)ccc7n6-c6ccccc6)c5n(-c5ccccc5)c34)nc(-c3ccccc3)n2)cc1] 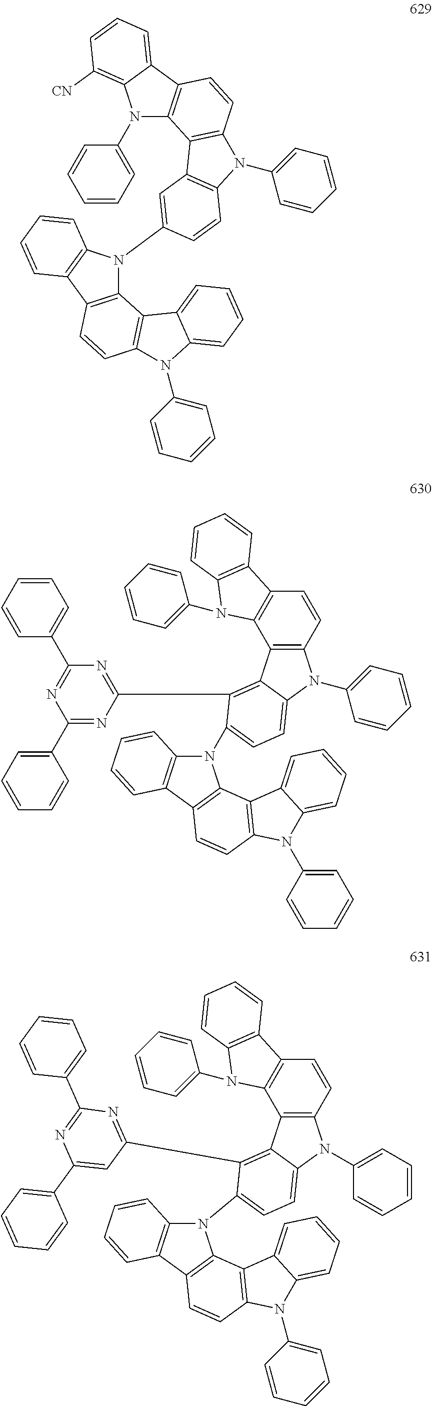 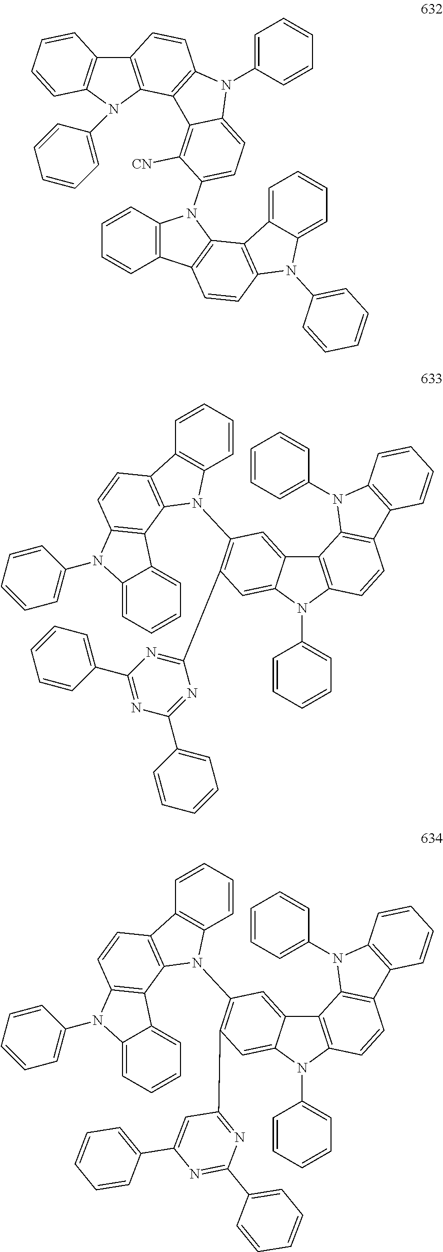 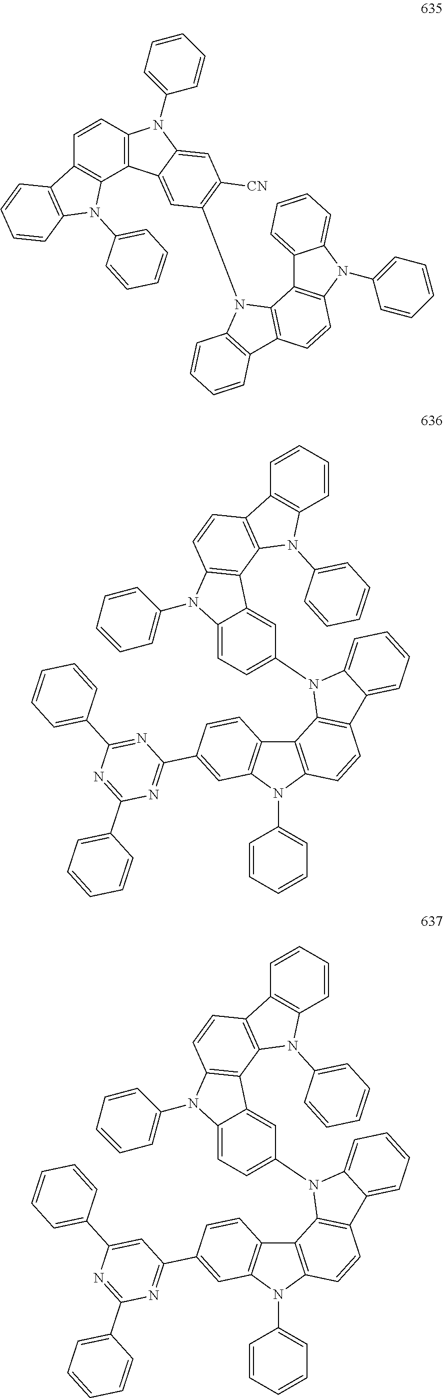 [SMILES: N#Cc1cc2c(cc1-n1c3ccccc3c3ccc4c(c5ccccc5n4-c4ccccc4)c31)c1c(ccc3c4ccccc4n(-c4ccccc4)c31)n2-c1ccccc1 ; c1ccc(-c2nc(-c3ccccc3)nc(-c3ccc4c5c(ccc6c7ccccc7n(-c7ccc8c(c7)c7c(ccc9c%10ccccc%10n(-c%10ccccc%10)c97)n8-c7ccccc7)c65)n(-c5ccccc5)c4c3)n2)cc1 ; c1ccc(-c2cc(-c3ccc4c5c(ccc6c7ccccc7n(-c7ccc8c(c7)c7c(ccc9c%10ccccc%10n(-c%10ccccc%10)c97)n8-c7ccccc7)c65)n(-c5ccccc5)c4c3)nc(-c3ccccc3)n2)cc1] 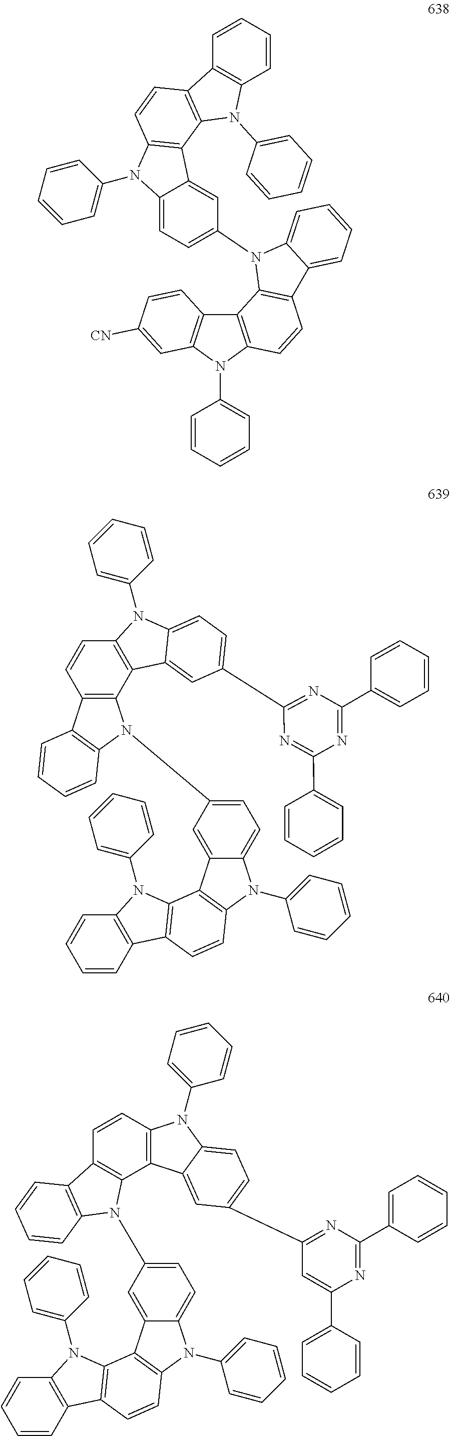 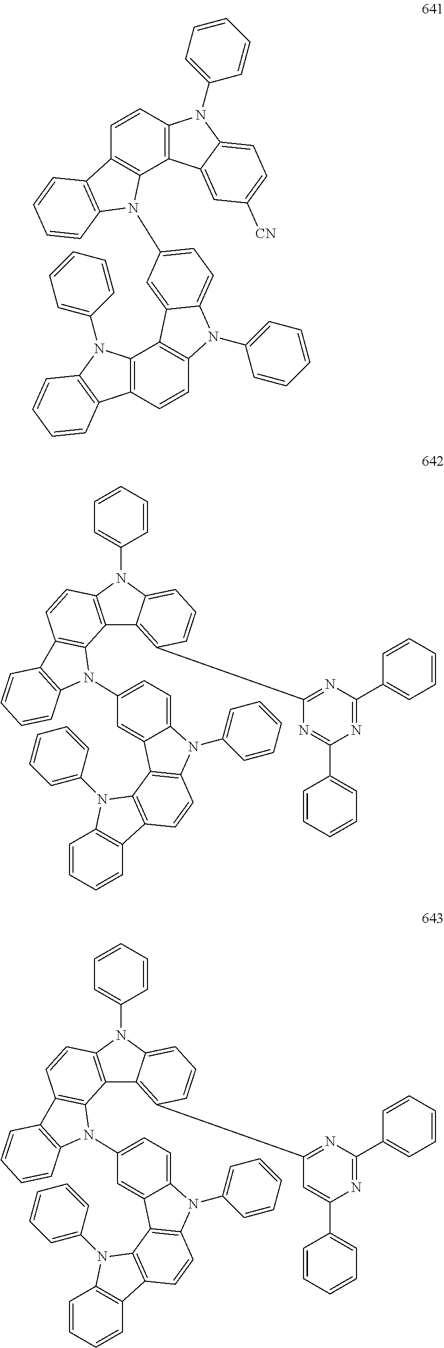 [SMILES: N#Cc1ccc2c(c1)c1c(ccc3c4ccccc4n(-c4ccc5c(c4)c4c(ccc6c7ccccc7n(-c7ccccc7)c64)n5-c4ccccc4)c31)n2-c1ccccc1 ; c1ccc(-c2nc(-c3ccccc3)nc(-c3cccc4c3c3c(ccc5c6ccccc6n(-c6ccc7c(c6)c6c(ccc8c9ccccc9n(-c9ccccc9)c86)n7-c6ccccc6)c53)n4-c3ccccc3)n2)cc1 ; c1ccc(-c2cc(-c3cccc4c3c3c(ccc5c6ccccc6n(-c6ccc7c(c6)c6c(ccc8c9ccccc9n(-c9ccccc9)c86)n7-c6ccccc6)c53)n4-c3ccccc3)nc(-c3ccccc3)n2)cc1] 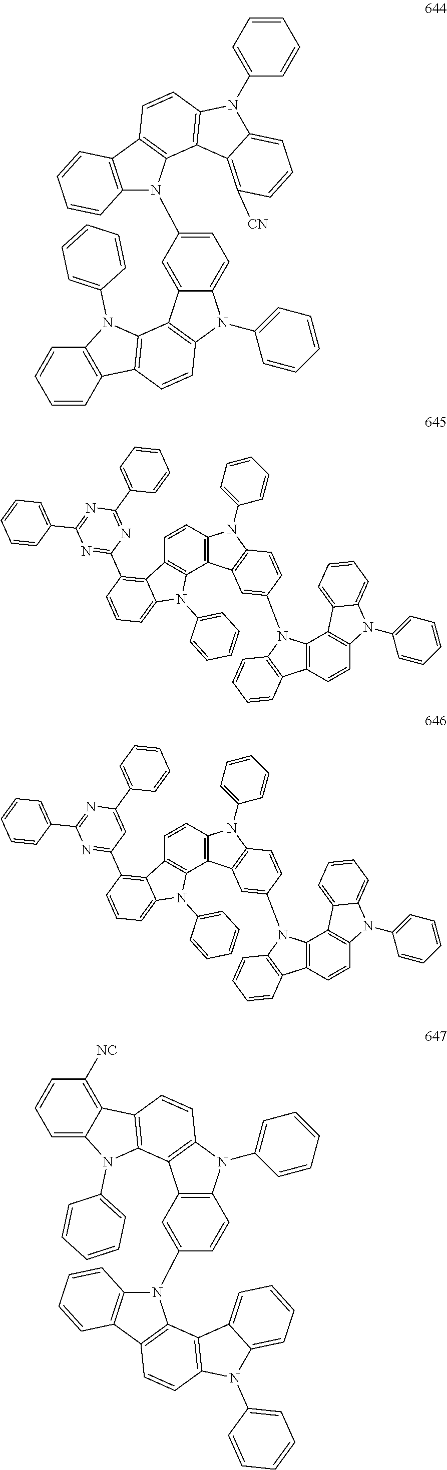 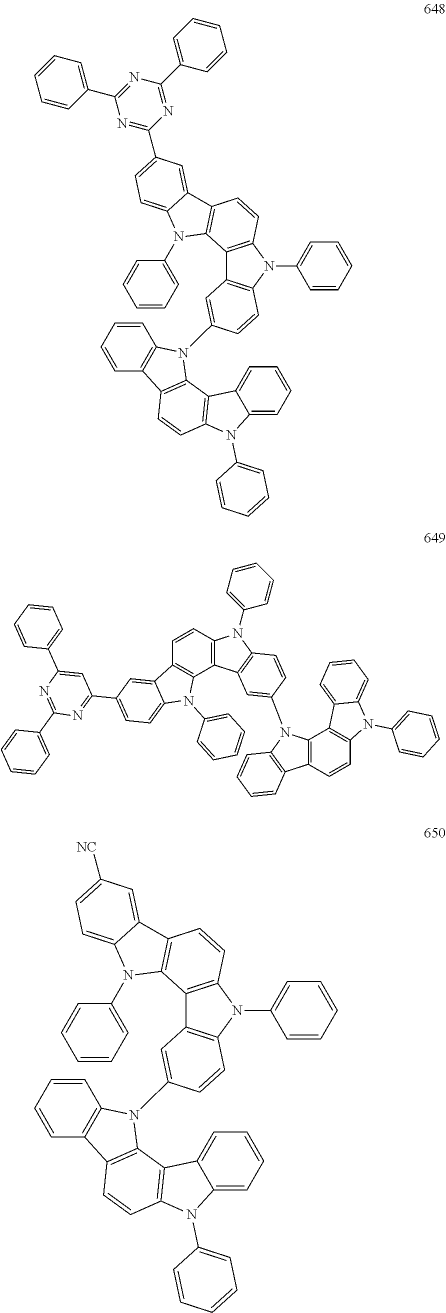 [SMILES: c1ccc(-c2nc(-c3ccccc3)nc(-c3ccc4c(c3)c3ccc5c(c6cc(-n7c8ccccc8c8ccc9c(c%10ccccc%10n9-c9ccccc9)c87)ccc6n5-c5ccccc5)c3n4-c3ccccc3)n2)cc1 ; c1ccc(-c2cc(-c3ccc4c(c3)c3ccc5c(c6cc(-n7c8ccccc8c8ccc9c(c%10ccccc%10n9-c9ccccc9)c87)ccc6n5-c5ccccc5)c3n4-c3ccccc3)nc(-c3ccccc3)n2)cc1 ; N#Cc1ccc2c(c1)c1ccc3c(c4cc(-n5c6ccccc6c6ccc7c(c8ccccc8n7-c7ccccc7)c65)ccc4n3-c3ccccc3)c1n2-c1ccccc1] 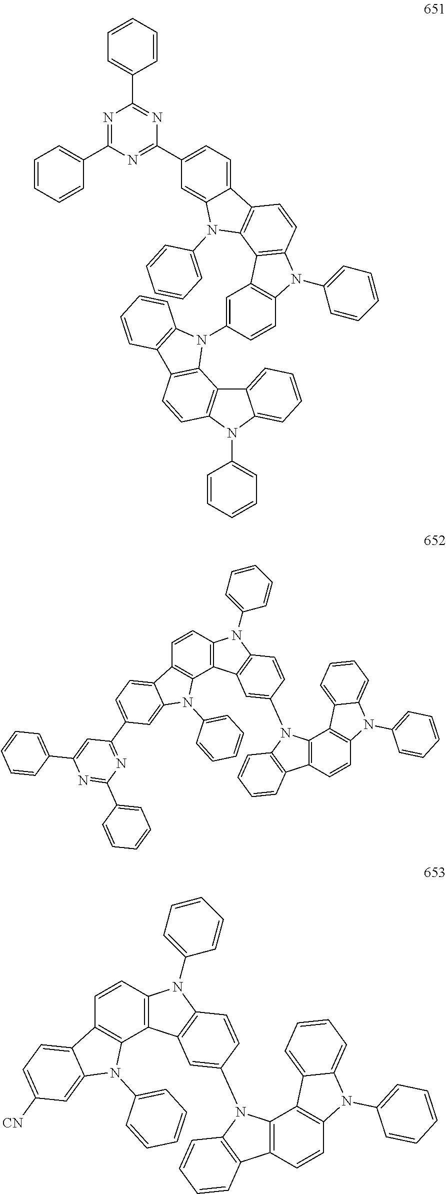 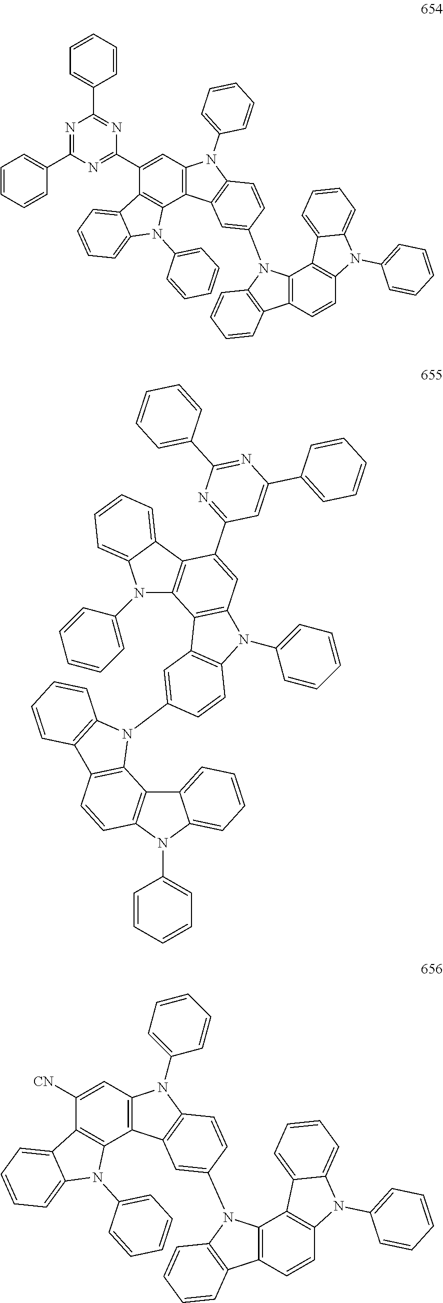 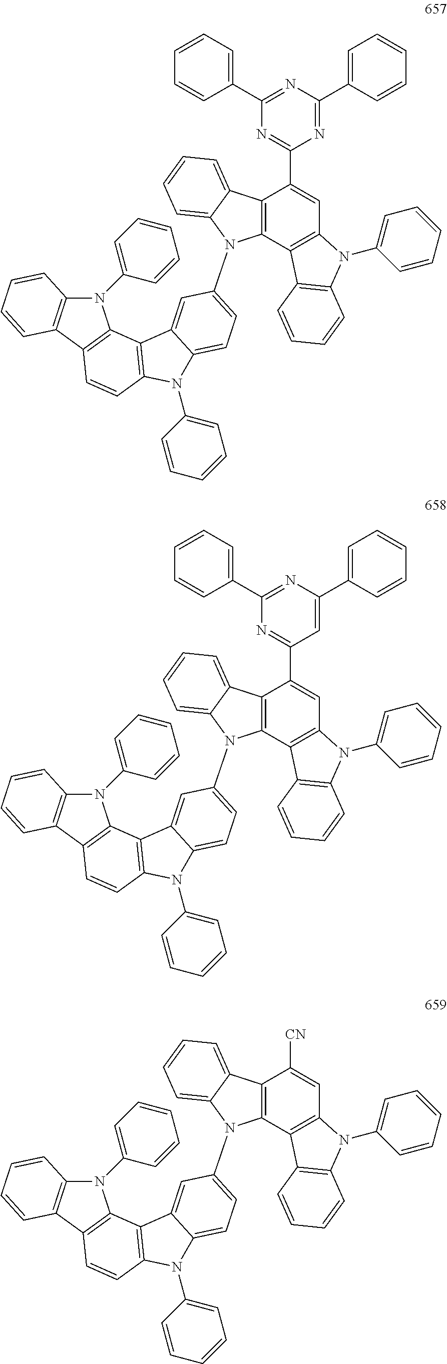 [SMILES: c1ccc(-c2nc(-c3ccccc3)nc(-c3cc4c(c5ccccc5n4-c4ccccc4)c4c3c3ccccc3n4-c3ccc4c(c3)c3c(ccc5c6ccccc6n(-c6ccccc6)c53)n4-c3ccccc3)n2)cc1 ; c1ccc(-c2cc(-c3cc4c(c5ccccc5n4-c4ccccc4)c4c3c3ccccc3n4-c3ccc4c(c3)c3c(ccc5c6ccccc6n(-c6ccccc6)c53)n4-c3ccccc3)nc(-c3ccccc3)n2)cc1 ; N#Cc1cc2c(c3ccccc3n2-c2ccccc2)c2c1c1ccccc1n2-c1ccc2c(c1)c1c(ccc3c4ccccc4n(-c4ccccc4)c31)n2-c1ccccc1] 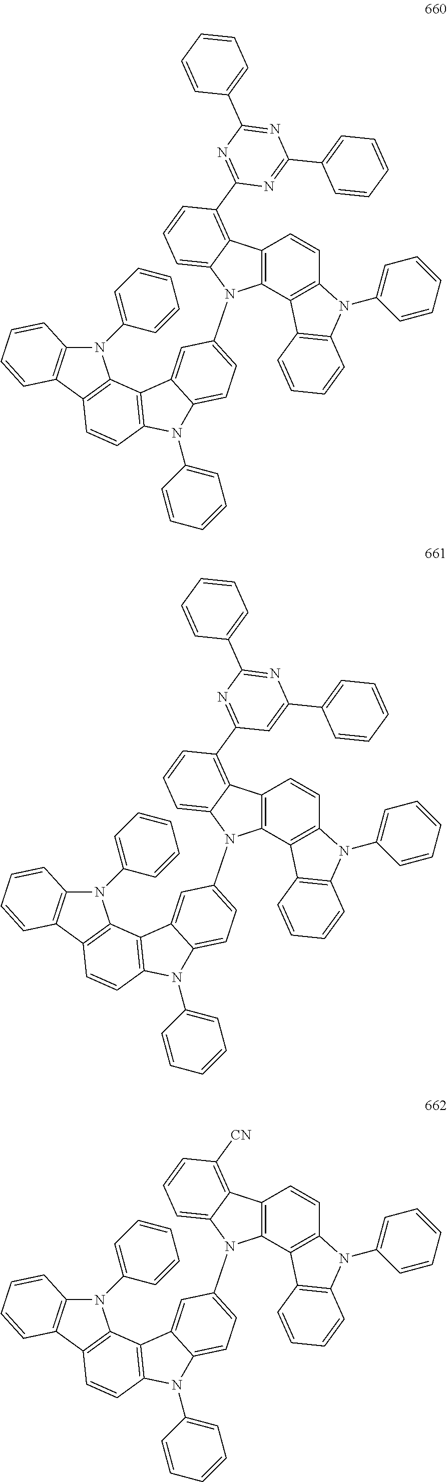 [SMILES: c1ccc(-c2nc(-c3ccccc3)nc(-c3cccc4c3c3ccc5c(c6ccccc6n5-c5ccccc5)c3n4-c3ccc4c(c3)c3c(ccc5c6ccccc6n(-c6ccccc6)c53)n4-c3ccccc3)n2)cc1 ; c1ccc(-c2cc(-c3cccc4c3c3ccc5c(c6ccccc6n5-c5ccccc5)c3n4-c3ccc4c(c3)c3c(ccc5c6ccccc6n(-c6ccccc6)c53)n4-c3ccccc3)nc(-c3ccccc3)n2)cc1 ; N#Cc1cccc2c1c1ccc3c(c4ccccc4n3-c3ccccc3)c1n2-c1ccc2c(c1)c1c(ccc3c4ccccc4n(-c4ccccc4)c31)n2-c1ccccc1] 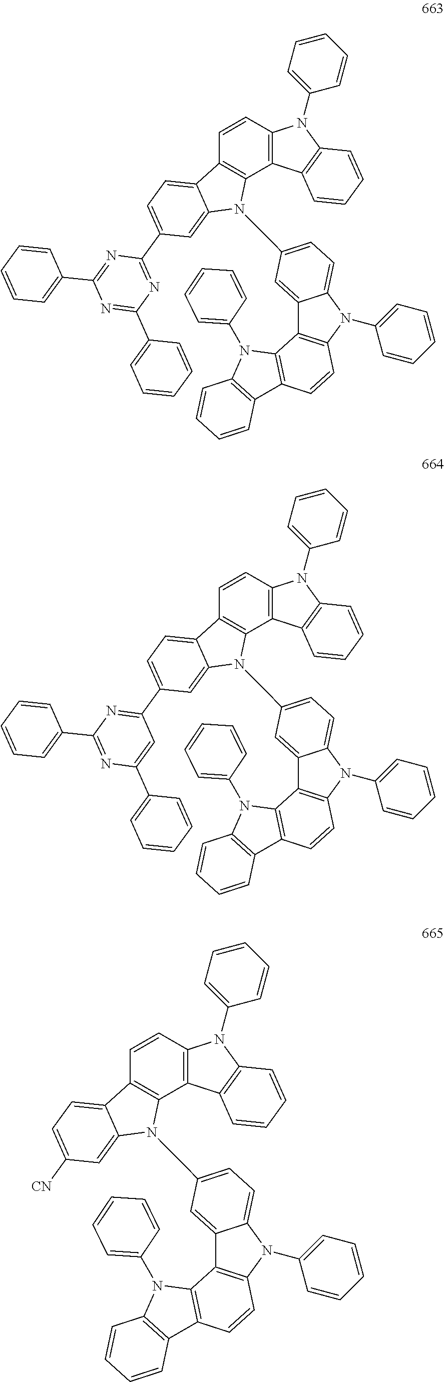 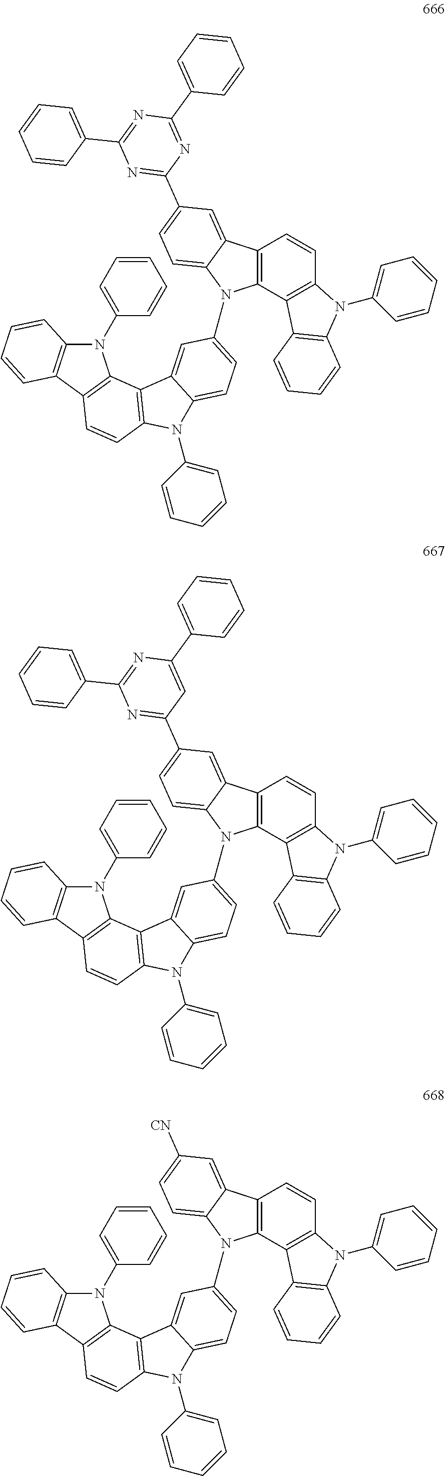 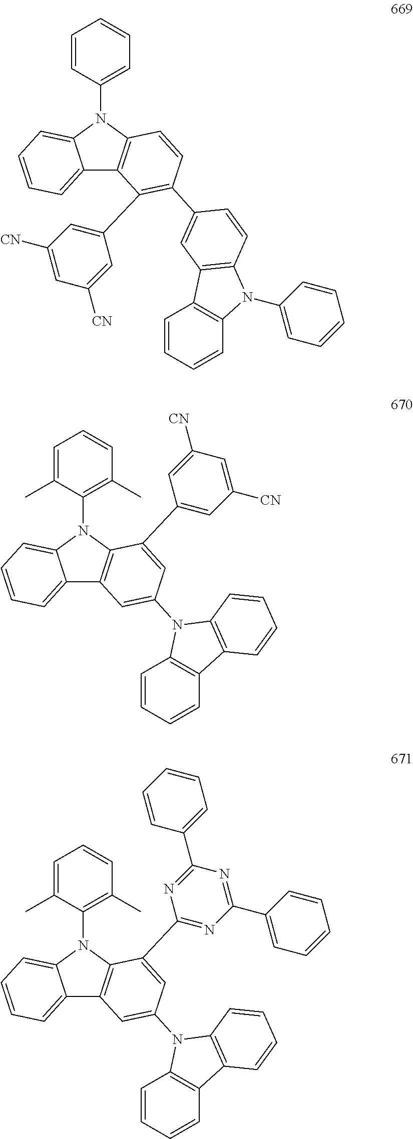 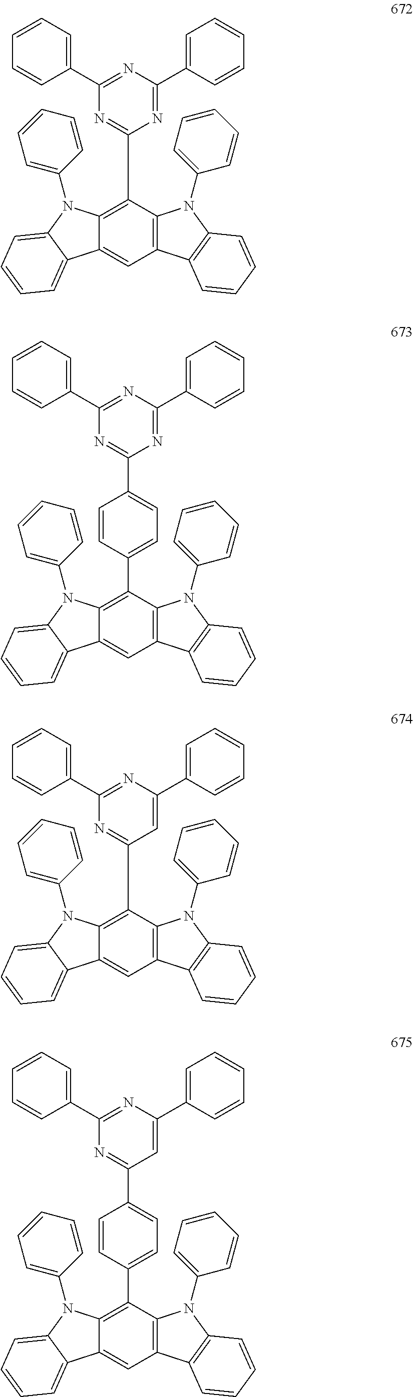 [SMILES: c1ccc(-c2nc(-c3ccccc3)nc(-c3c4c(cc5c6ccccc6n(-c6ccccc6)c35)c3ccccc3n4-c3ccccc3)n2)cc1 ; c1ccc(-c2nc(-c3ccccc3)nc(-c3ccc(-c4c5c(cc6c7ccccc7n(-c7ccccc7)c46)c4ccccc4n5-c4ccccc4)cc3)n2)cc1 ; c1ccc(-c2cc(-c3c4c(cc5c6ccccc6n(-c6ccccc6)c35)c3ccccc3n4-c3ccccc3)nc(-c3ccccc3)n2)cc1 ; c1ccc(-c2cc(-c3ccc(-c4c5c(cc6c7ccccc7n(-c7ccccc7)c46)c4ccccc4n5-c4ccccc4)cc3)nc(-c3ccccc3)n2)cc1] 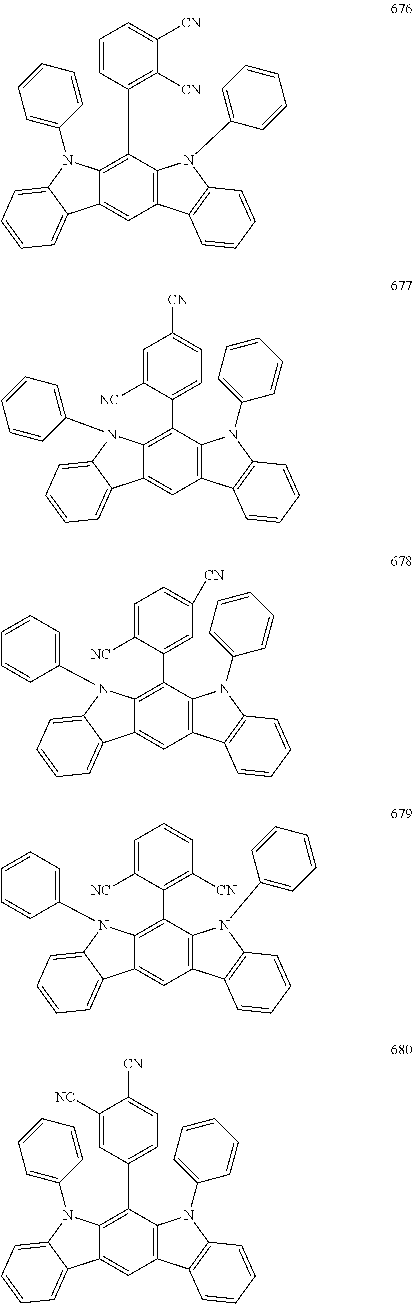 [SMILES: N#Cc1cccc(-c2c3c(cc4c5ccccc5n(-c5ccccc5)c24)c2ccccc2n3-c2ccccc2)c1C#N ; N#Cc1ccc(-c2c3c(cc4c5ccccc5n(-c5ccccc5)c24)c2ccccc2n3-c2ccccc2)c(C#N)c1 ; N#Cc1ccc(C#N)c(-c2c3c(cc4c5ccccc5n(-c5ccccc5)c24)c2ccccc2n3-c2ccccc2)c1 ; N#Cc1cccc(C#N)c1-c1c2c(cc3c4ccccc4n(-c4ccccc4)c13)c1ccccc1n2-c1ccccc1 ; N#Cc1ccc(-c2c3c(cc4c5ccccc5n(-c5ccccc5)c24)c2ccccc2n3-c2ccccc2)cc1C#N] 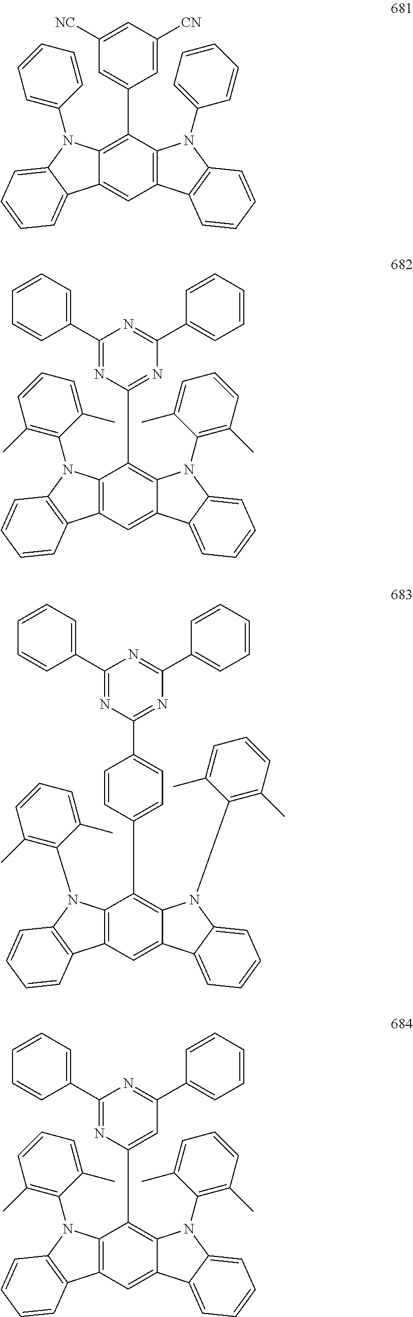 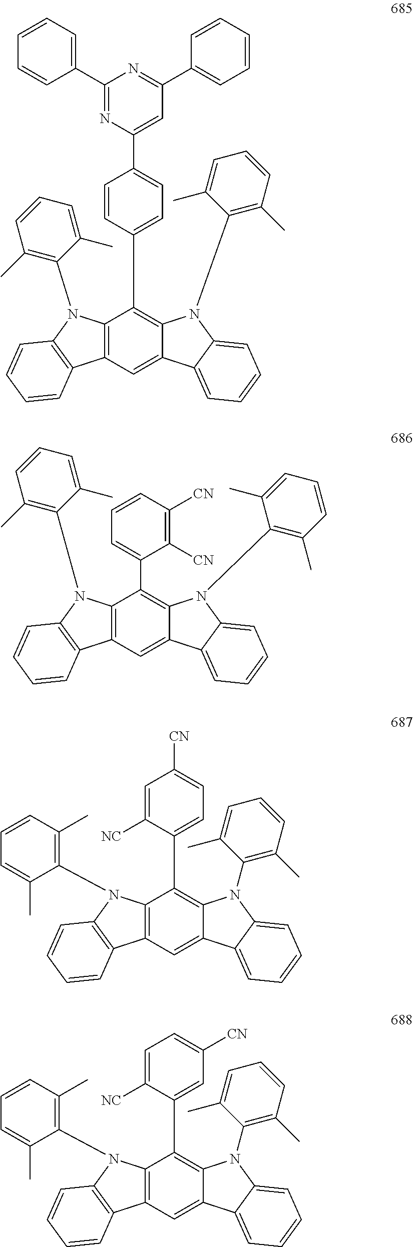 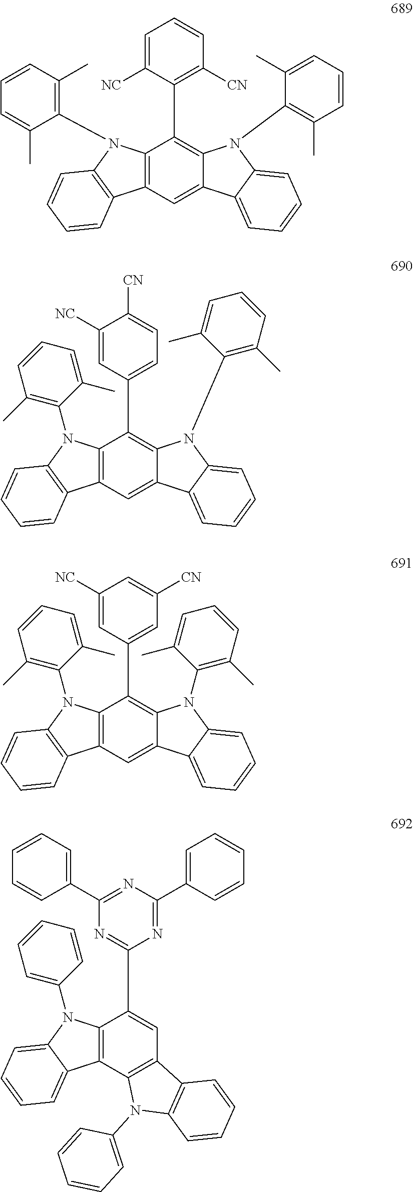 [SMILES: Cc1cccc(C)c1-n1c2ccccc2c2cc3c4ccccc4n(-c4c(C)cccc4C)c3c(-c3c(C#N)cccc3C#N)c21 ; Cc1cccc(C)c1-n1c2ccccc2c2cc3c4ccccc4n(-c4c(C)cccc4C)c3c(-c3ccc(C#N)c(C#N)c3)c21 ; Cc1cccc(C)c1-n1c2ccccc2c2cc3c4ccccc4n(-c4c(C)cccc4C)c3c(-c3cc(C#N)cc(C#N)c3)c21 ; c1ccc(-c2nc(-c3ccccc3)nc(-c3cc4c5ccccc5n(-c5ccccc5)c4c4c5ccccc5n(-c5ccccc5)c34)n2)cc1] 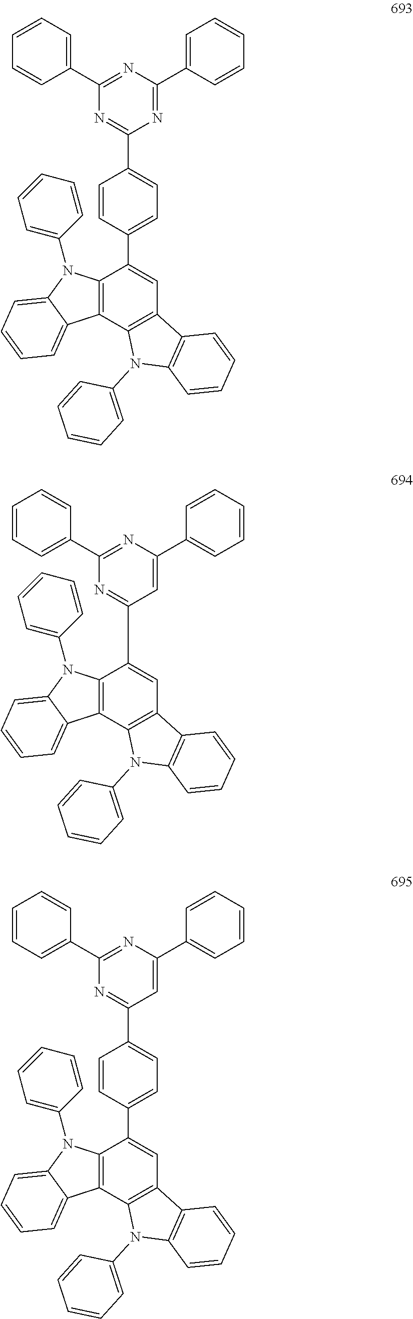 [SMILES: c1ccc(-c2nc(-c3ccccc3)nc(-c3ccc(-c4cc5c6ccccc6n(-c6ccccc6)c5c5c6ccccc6n(-c6ccccc6)c45)cc3)n2)cc1 ; c1ccc(-c2cc(-c3cc4c5ccccc5n(-c5ccccc5)c4c4c5ccccc5n(-c5ccccc5)c34)nc(-c3ccccc3)n2)cc1 ; c1ccc(-c2cc(-c3ccc(-c4cc5c6ccccc6n(-c6ccccc6)c5c5c6ccccc6n(-c6ccccc6)c45)cc3)nc(-c3ccccc3)n2)cc1] 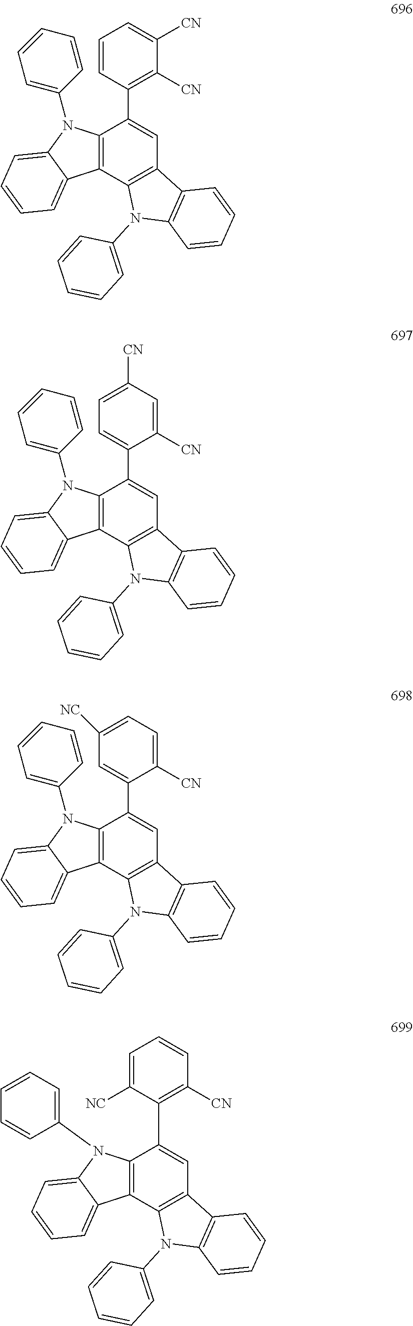 [SMILES: N#Cc1cccc(-c2cc3c4ccccc4n(-c4ccccc4)c3c3c4ccccc4n(-c4ccccc4)c23)c1C#N ; N#Cc1ccc(-c2cc3c4ccccc4n(-c4ccccc4)c3c3c4ccccc4n(-c4ccccc4)c23)c(C#N)c1 ; N#Cc1ccc(C#N)c(-c2cc3c4ccccc4n(-c4ccccc4)c3c3c4ccccc4n(-c4ccccc4)c23)c1 ; N#Cc1cccc(C#N)c1-c1cc2c3ccccc3n(-c3ccccc3)c2c2c3ccccc3n(-c3ccccc3)c12] 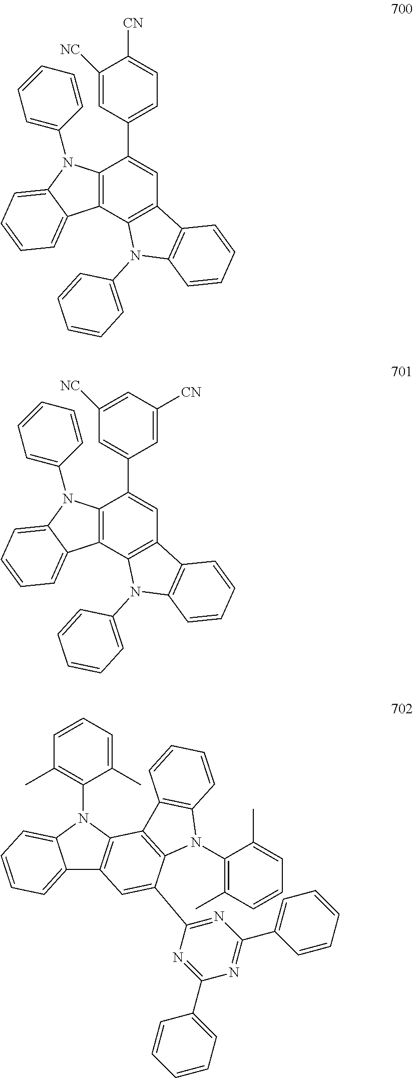 [SMILES: N#Cc1ccc(-c2cc3c4ccccc4n(-c4ccccc4)c3c3c4ccccc4n(-c4ccccc4)c23)cc1C#N ; N#Cc1cc(C#N)cc(-c2cc3c4ccccc4n(-c4ccccc4)c3c3c4ccccc4n(-c4ccccc4)c23)c1 ; Cc1cccc(C)c1-n1c2ccccc2c2c1c(-c1nc(-c3ccccc3)nc(-c3ccccc3)n1)cc1c3ccccc3n(-c3c(C)cccc3C)c12] 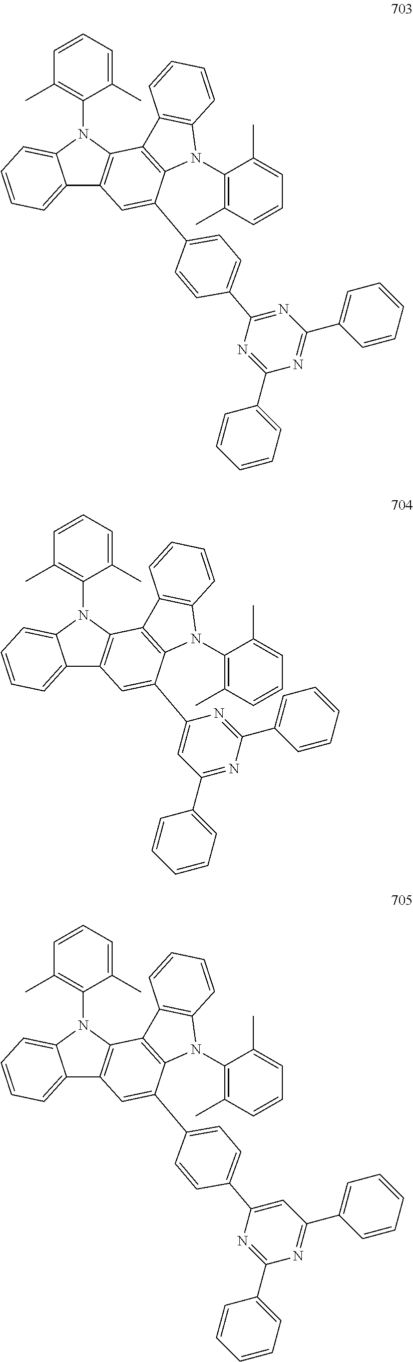 [SMILES: Cc1cccc(C)c1-n1c2ccccc2c2c1c(-c1ccc(-c3nc(-c4ccccc4)nc(-c4ccccc4)n3)cc1)cc1c3ccccc3n(-c3c(C)cccc3C)c12 ; Cc1cccc(C)c1-n1c2ccccc2c2c1c(-c1cc(-c3ccccc3)nc(-c3ccccc3)n1)cc1c3ccccc3n(-c3c(C)cccc3C)c12 ; Cc1cccc(C)c1-n1c2ccccc2c2c1c(-c1ccc(-c3cc(-c4ccccc4)nc(-c4ccccc4)n3)cc1)cc1c3ccccc3n(-c3c(C)cccc3C)c12] 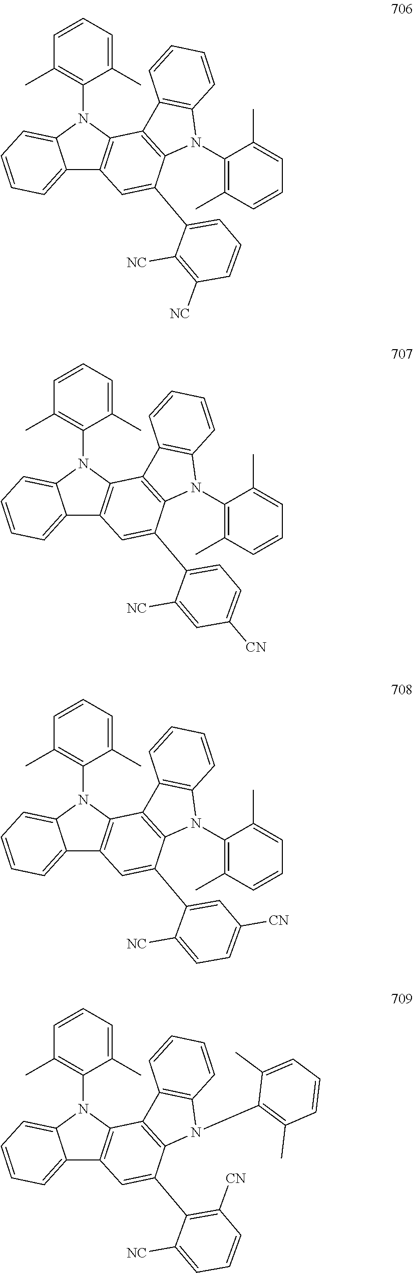 [SMILES: Cc1cccc(C)c1-n1c2ccccc2c2c1c(-c1cccc(C#N)c1C#N)cc1c3ccccc3n(-c3c(C)cccc3C)c12 ; Cc1cccc(C)c1-n1c2ccccc2c2c1c(-c1ccc(C#N)cc1C#N)cc1c3ccccc3n(-c3c(C)cccc3C)c12 ; Cc1cccc(C)c1-n1c2ccccc2c2c1c(-c1cc(C#N)ccc1C#N)cc1c3ccccc3n(-c3c(C)cccc3C)c12 ; Cc1cccc(C)c1-n1c2ccccc2c2c1c(-c1c(C#N)cccc1C#N)cc1c3ccccc3n(-c3c(C)cccc3C)c12] 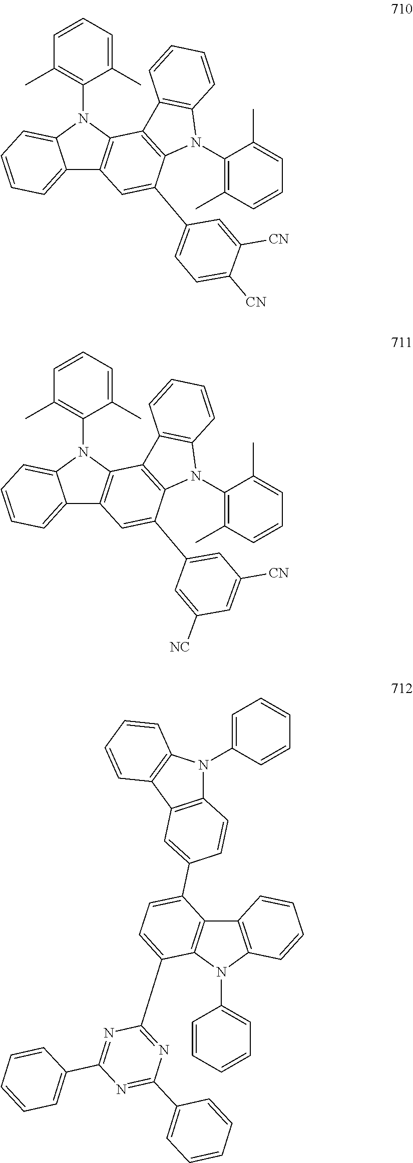 [SMILES: Cc1cccc(C)c1-n1c2ccccc2c2c1c(-c1ccc(C#N)c(C#N)c1)cc1c3ccccc3n(-c3c(C)cccc3C)c12 ; Cc1cccc(C)c1-n1c2ccccc2c2c1c(-c1cc(C#N)cc(C#N)c1)cc1c3ccccc3n(-c3c(C)cccc3C)c12 ; c1ccc(-c2nc(-c3ccccc3)nc(-c3ccc(-c4ccc5c(c4)c4ccccc4n5-c4ccccc4)c4c5ccccc5n(-c5ccccc5)c34)n2)cc1] 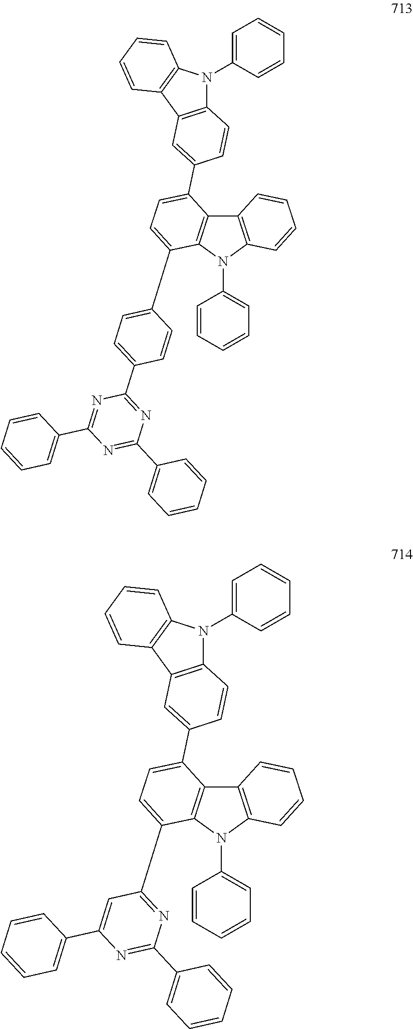 [SMILES: c1ccc(-c2nc(-c3ccccc3)nc(-c3ccc(-c4ccc(-c5ccc6c(c5)c5ccccc5n6-c5ccccc5)c5c6ccccc6n(-c6ccccc6)c45)cc3)n2)cc1 ; c1ccc(-c2cc(-c3ccc(-c4ccc5c(c4)c4ccccc4n5-c4ccccc4)c4c5ccccc5n(-c5ccccc5)c34)nc(-c3ccccc3)n2)cc1] 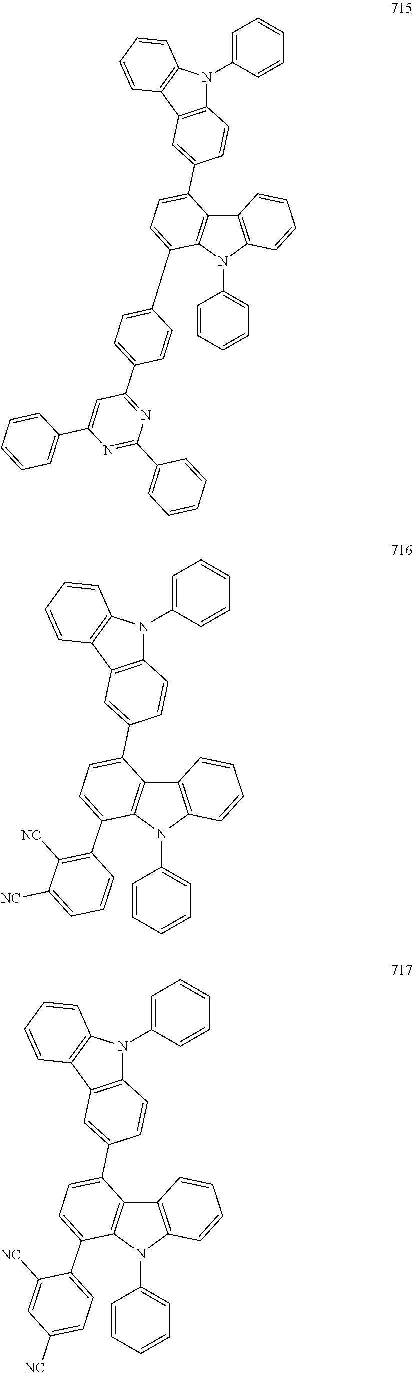 [SMILES: c1ccc(-c2cc(-c3ccc(-c4ccc(-c5ccc6c(c5)c5ccccc5n6-c5ccccc5)c5c6ccccc6n(-c6ccccc6)c45)cc3)nc(-c3ccccc3)n2)cc1 ; N#Cc1cccc(-c2ccc(-c3ccc4c(c3)c3ccccc3n4-c3ccccc3)c3c4ccccc4n(-c4ccccc4)c23)c1C#N ; N#Cc1ccc(-c2ccc(-c3ccc4c(c3)c3ccccc3n4-c3ccccc3)c3c4ccccc4n(-c4ccccc4)c23)c(C#N)c1] 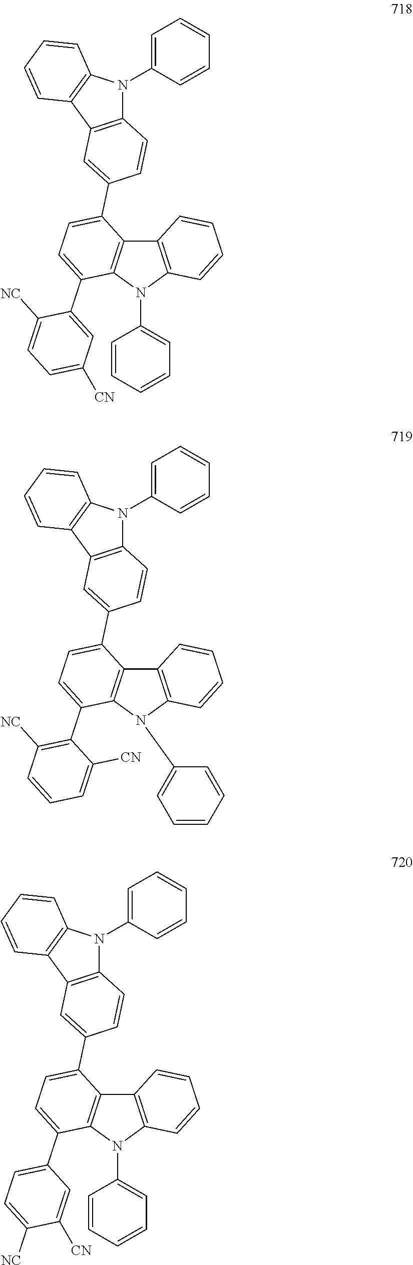 [SMILES: N#Cc1ccc(C#N)c(-c2ccc(-c3ccc4c(c3)c3ccccc3n4-c3ccccc3)c3c4ccccc4n(-c4ccccc4)c23)c1 ; N#Cc1cccc(C#N)c1-c1ccc(-c2ccc3c(c2)c2ccccc2n3-c2ccccc2)c2c3ccccc3n(-c3ccccc3)c12 ; N#Cc1ccc(-c2ccc(-c3ccc4c(c3)c3ccccc3n4-c3ccccc3)c3c4ccccc4n(-c4ccccc4)c23)cc1C#N] 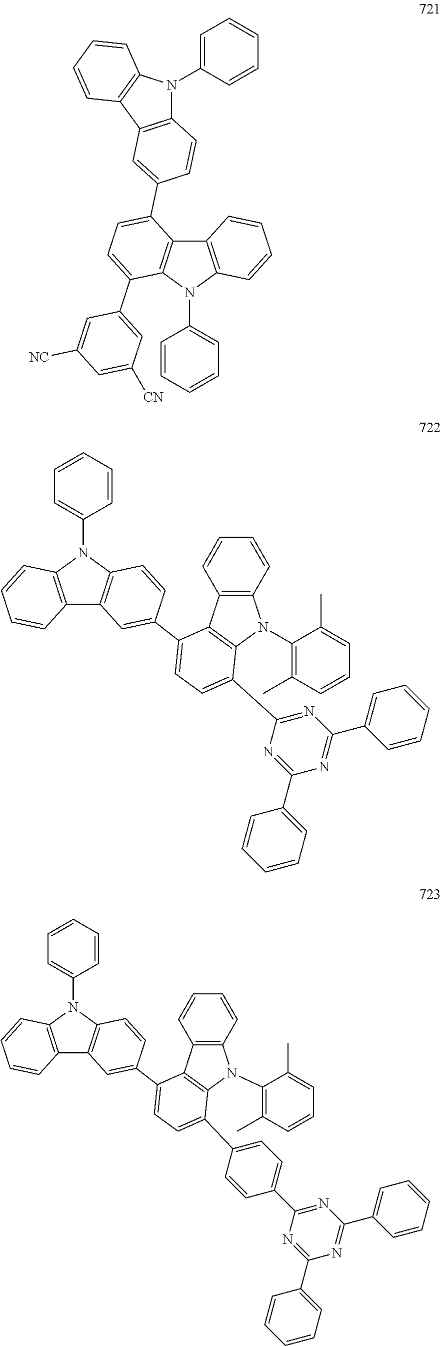 [SMILES: N#Cc1cc(C#N)cc(-c2ccc(-c3ccc4c(c3)c3ccccc3n4-c3ccccc3)c3c4ccccc4n(-c4ccccc4)c23)c1 ; Cc1cccc(C)c1-n1c2ccccc2c2c(-c3ccc4c(c3)c3ccccc3n4-c3ccccc3)ccc(-c3nc(-c4ccccc4)nc(-c4ccccc4)n3)c21 ; Cc1cccc(C)c1-n1c2ccccc2c2c(-c3ccc4c(c3)c3ccccc3n4-c3ccccc3)ccc(-c3ccc(-c4nc(-c5ccccc5)nc(-c5ccccc5)n4)cc3)c21] 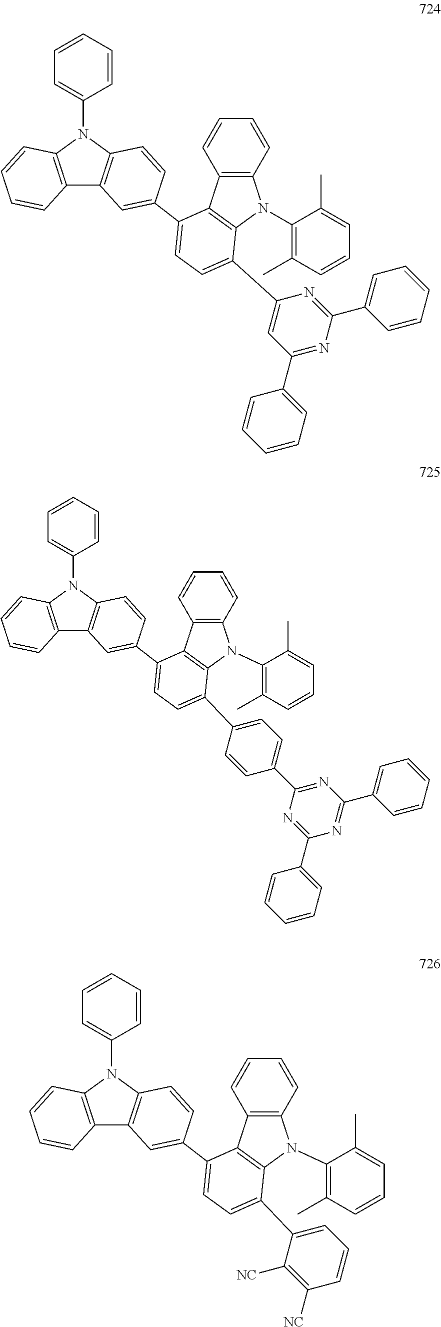 [SMILES: Cc1cccc(C)c1-n1c2ccccc2c2c(-c3ccc4c(c3)c3ccccc3n4-c3ccccc3)ccc(-c3cc(-c4ccccc4)nc(-c4ccccc4)n3)c21 ; Cc1cccc(C)c1-n1c2ccccc2c2c(-c3ccc4c(c3)c3ccccc3n4-c3ccccc3)ccc(-c3ccc(-c4nc(-c5ccccc5)nc(-c5ccccc5)n4)cc3)c21 ; Cc1cccc(C)c1-n1c2ccccc2c2c(-c3ccc4c(c3)c3ccccc3n4-c3ccccc3)ccc(-c3cccc(C#N)c3C#N)c21] 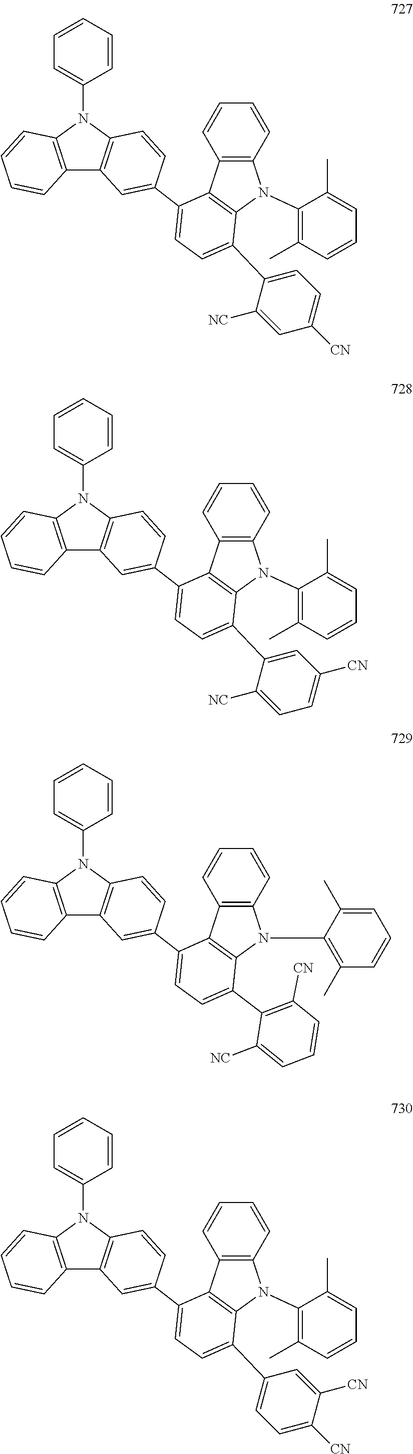 [SMILES: Cc1cccc(C)c1-n1c2ccccc2c2c(-c3ccc4c(c3)c3ccccc3n4-c3ccccc3)ccc(-c3ccc(C#N)cc3C#N)c21 ; Cc1cccc(C)c1-n1c2ccccc2c2c(-c3ccc4c(c3)c3ccccc3n4-c3ccccc3)ccc(-c3cc(C#N)ccc3C#N)c21 ; Cc1cccc(C)c1-n1c2ccccc2c2c(-c3ccc4c(c3)c3ccccc3n4-c3ccccc3)ccc(-c3c(C#N)cccc3C#N)c21 ; Cc1cccc(C)c1-n1c2ccccc2c2c(-c3ccc4c(c3)c3ccccc3n4-c3ccccc3)ccc(-c3ccc(C#N)c(C#N)c3)c21] 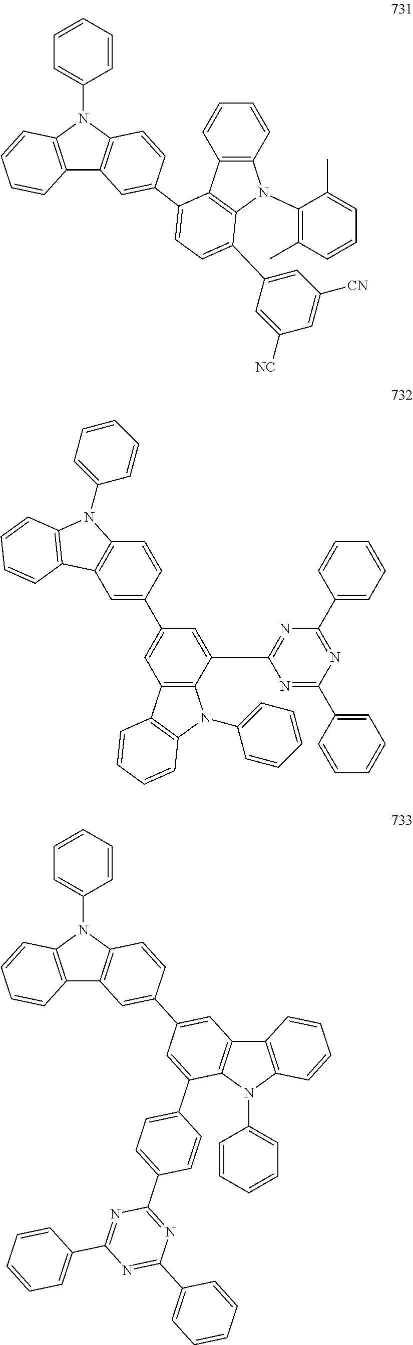 [SMILES: Cc1cccc(C)c1-n1c2ccccc2c2c(-c3ccc4c(c3)c3ccccc3n4-c3ccccc3)ccc(-c3cc(C#N)cc(C#N)c3)c21 ; c1ccc(-c2nc(-c3ccccc3)nc(-c3cc(-c4ccc5c(c4)c4ccccc4n5-c4ccccc4)cc4c5ccccc5n(-c5ccccc5)c34)n2)cc1 ; c1ccc(-c2nc(-c3ccccc3)nc(-c3ccc(-c4cc(-c5ccc6c(c5)c5ccccc5n6-c5ccccc5)cc5c6ccccc6n(-c6ccccc6)c45)cc3)n2)cc1] 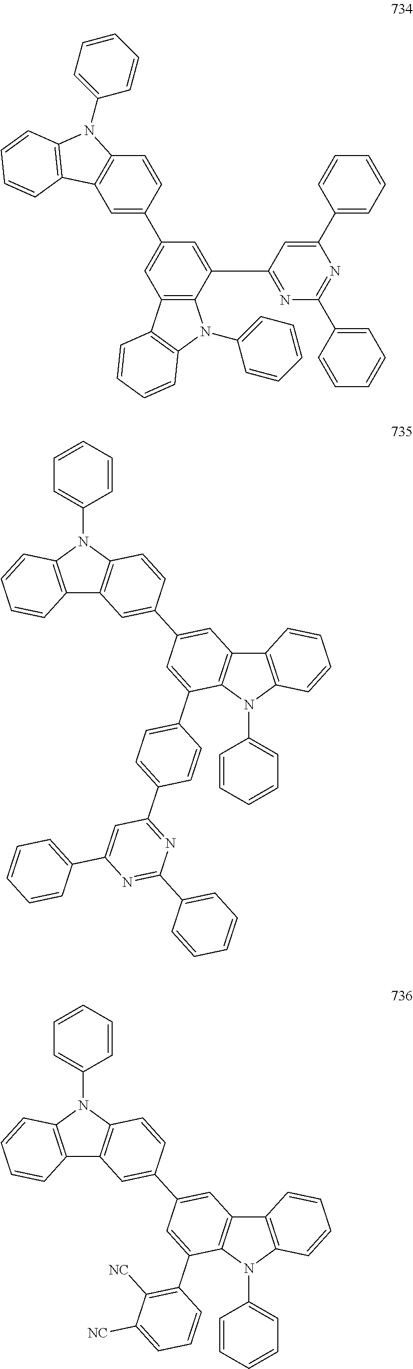 [SMILES: c1ccc(-c2cc(-c3cc(-c4ccc5c(c4)c4ccccc4n5-c4ccccc4)cc4c5ccccc5n(-c5ccccc5)c34)nc(-c3ccccc3)n2)cc1 ; c1ccc(-c2cc(-c3ccc(-c4cc(-c5ccc6c(c5)c5ccccc5n6-c5ccccc5)cc5c6ccccc6n(-c6ccccc6)c45)cc3)nc(-c3ccccc3)n2)cc1 ; N#Cc1cccc(-c2cc(-c3ccc4c(c3)c3ccccc3n4-c3ccccc3)cc3c4ccccc4n(-c4ccccc4)c23)c1C#N] 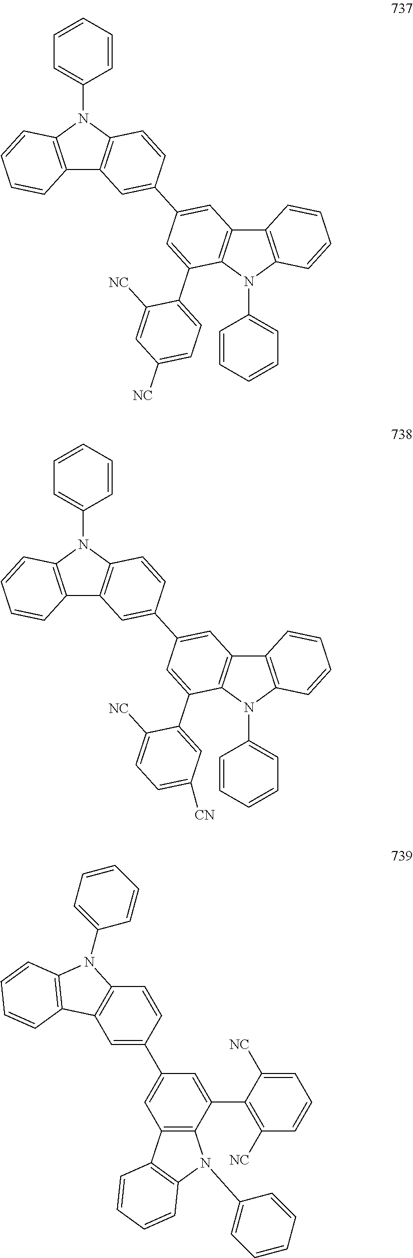 [SMILES: N#Cc1ccc(-c2cc(-c3ccc4c(c3)c3ccccc3n4-c3ccccc3)cc3c4ccccc4n(-c4ccccc4)c23)c(C#N)c1 ; N#Cc1ccc(C#N)c(-c2cc(-c3ccc4c(c3)c3ccccc3n4-c3ccccc3)cc3c4ccccc4n(-c4ccccc4)c23)c1 ; N#Cc1cccc(C#N)c1-c1cc(-c2ccc3c(c2)c2ccccc2n3-c2ccccc2)cc2c3ccccc3n(-c3ccccc3)c12] 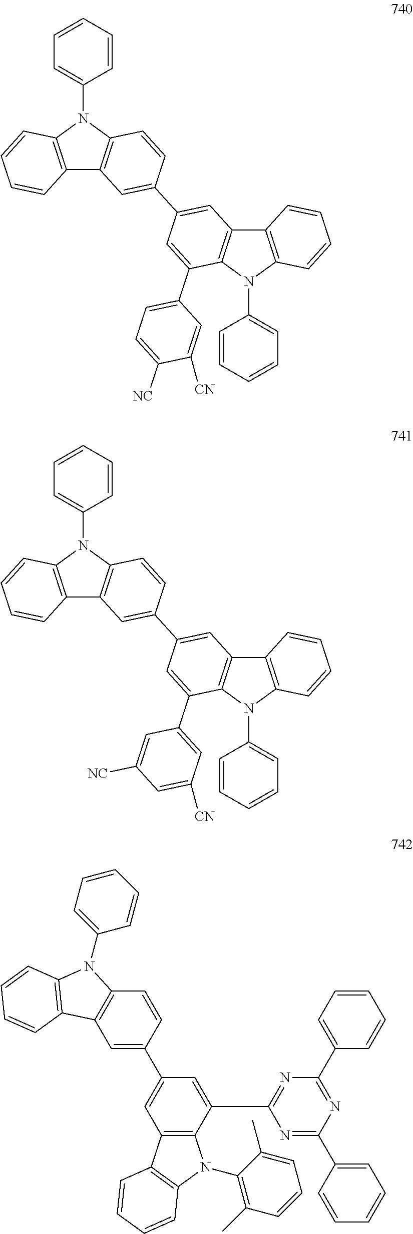 [SMILES: N#Cc1ccc(-c2cc(-c3ccc4c(c3)c3ccccc3n4-c3ccccc3)cc3c4ccccc4n(-c4ccccc4)c23)cc1C#N ; N#Cc1cc(C#N)cc(-c2cc(-c3ccc4c(c3)c3ccccc3n4-c3ccccc3)cc3c4ccccc4n(-c4ccccc4)c23)c1 ; Cc1cccc(C)c1-n1c2ccccc2c2cc(-c3ccc4c(c3)c3ccccc3n4-c3ccccc3)cc(-c3nc(-c4ccccc4)nc(-c4ccccc4)n3)c21] 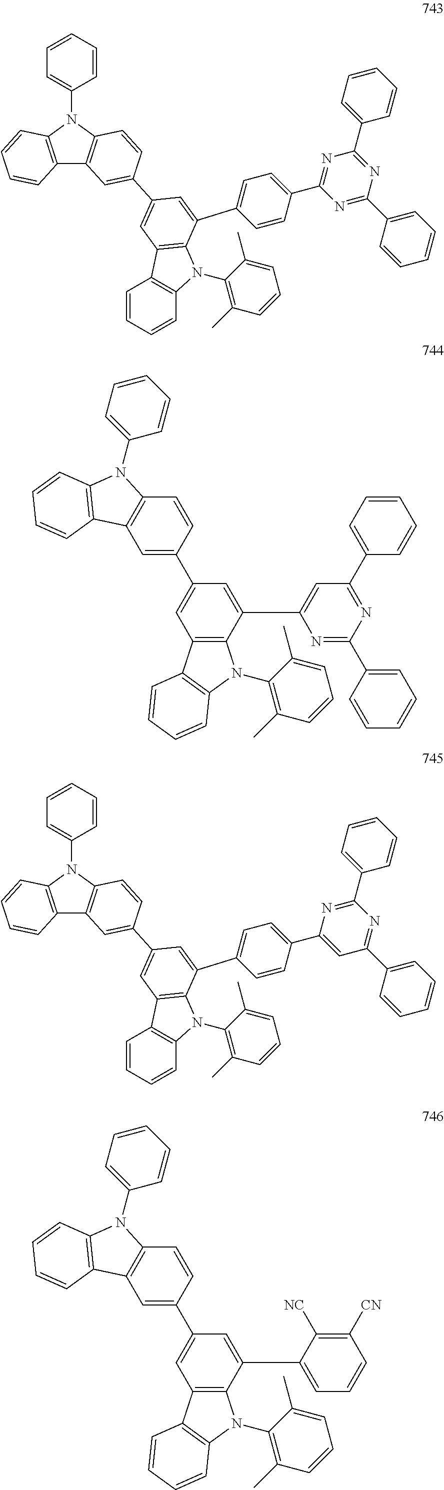 [SMILES: Cc1cccc(C)c1-n1c2ccccc2c2cc(-c3ccc4c(c3)c3ccccc3n4-c3ccccc3)cc(-c3ccc(-c4nc(-c5ccccc5)nc(-c5ccccc5)n4)cc3)c21 ; Cc1cccc(C)c1-n1c2ccccc2c2cc(-c3ccc4c(c3)c3ccccc3n4-c3ccccc3)cc(-c3cc(-c4ccccc4)nc(-c4ccccc4)n3)c21 ; Cc1cccc(C)c1-n1c2ccccc2c2cc(-c3ccc4c(c3)c3ccccc3n4-c3ccccc3)cc(-c3ccc(-c4cc(-c5ccccc5)nc(-c5ccccc5)n4)cc3)c21 ; Cc1cccc(C)c1-n1c2ccccc2c2cc(-c3ccc4c(c3)c3ccccc3n4-c3ccccc3)cc(-c3cccc(C#N)c3C#N)c21] 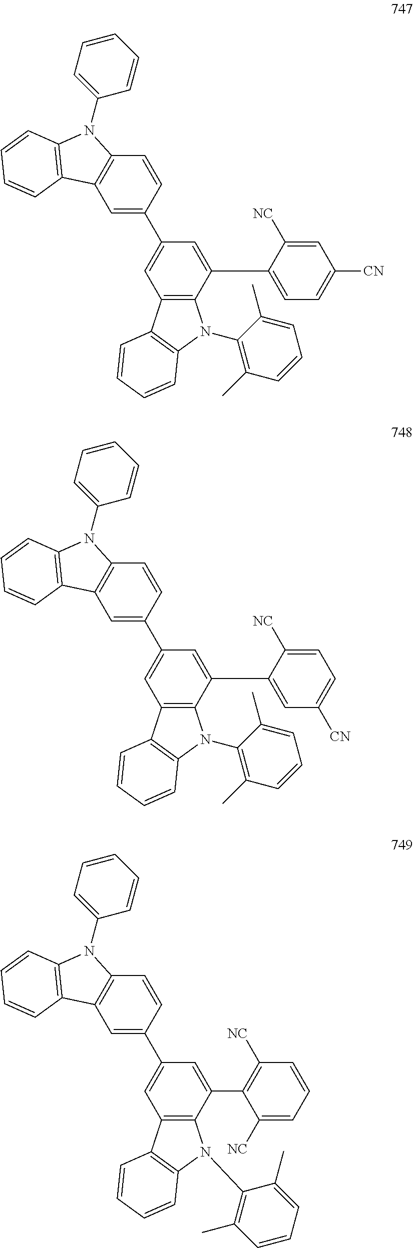 [SMILES: Cc1cccc(C)c1-n1c2ccccc2c2cc(-c3ccc4c(c3)c3ccccc3n4-c3ccccc3)cc(-c3ccc(C#N)cc3C#N)c21 ; Cc1cccc(C)c1-n1c2ccccc2c2cc(-c3ccc4c(c3)c3ccccc3n4-c3ccccc3)cc(-c3cc(C#N)ccc3C#N)c21 ; Cc1cccc(C)c1-n1c2ccccc2c2cc(-c3ccc4c(c3)c3ccccc3n4-c3ccccc3)cc(-c3c(C#N)cccc3C#N)c21] 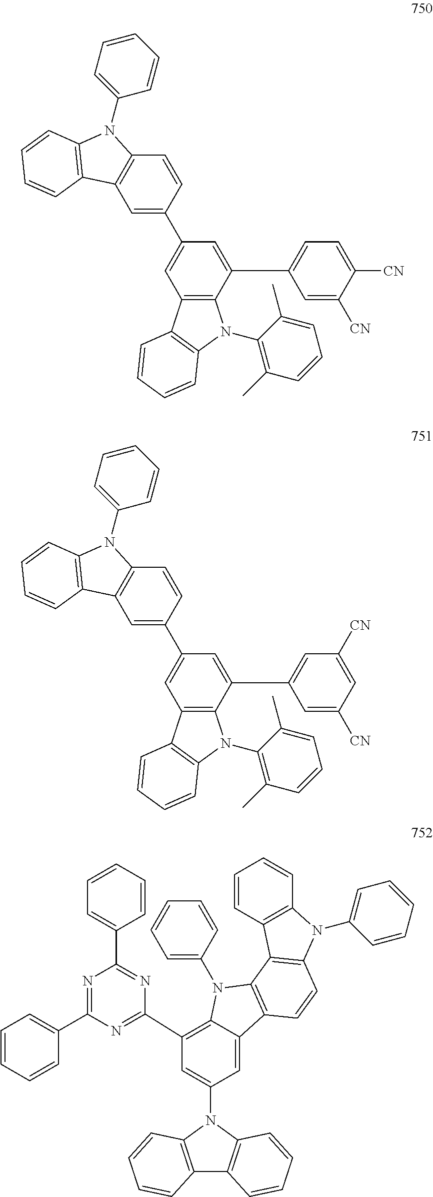 [SMILES: Cc1cccc(C)c1-n1c2ccccc2c2cc(-c3ccc4c(c3)c3ccccc3n4-c3ccccc3)cc(-c3ccc(C#N)c(C#N)c3)c21 ; Cc1cccc(C)c1-n1c2ccccc2c2cc(-c3ccc4c(c3)c3ccccc3n4-c3ccccc3)cc(-c3cc(C#N)cc(C#N)c3)c21 ; c1ccc(-c2nc(-c3ccccc3)nc(-c3cc(-n4c5ccccc5c5ccccc54)cc4c5ccc6c(c7ccccc7n6-c6ccccc6)c5n(-c5ccccc5)c34)n2)cc1] 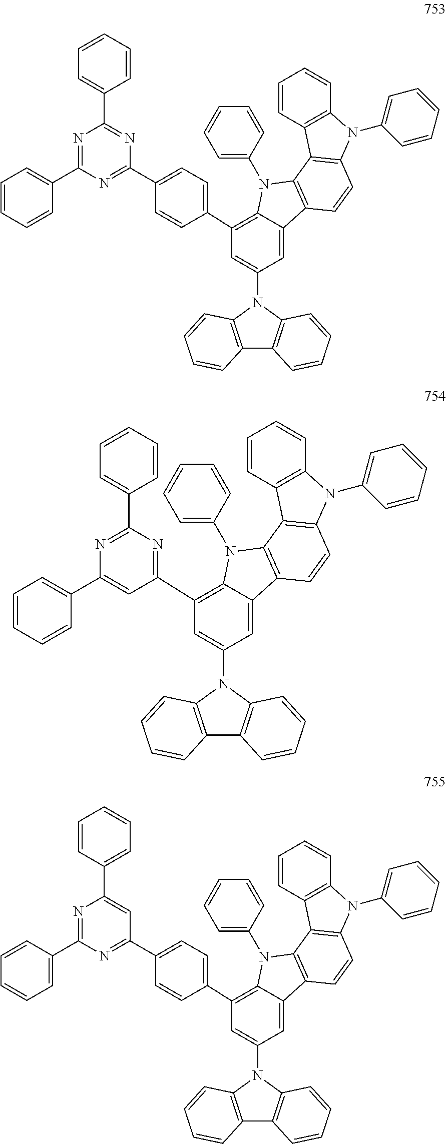 [SMILES: c1ccc(-c2nc(-c3ccccc3)nc(-c3ccc(-c4cc(-n5c6ccccc6c6ccccc65)cc5c6ccc7c(c8ccccc8n7-c7ccccc7)c6n(-c6ccccc6)c45)cc3)n2)cc1 ; c1ccc(-c2cc(-c3cc(-n4c5ccccc5c5ccccc54)cc4c5ccc6c(c7ccccc7n6-c6ccccc6)c5n(-c5ccccc5)c34)nc(-c3ccccc3)n2)cc1 ; c1ccc(-c2cc(-c3ccc(-c4cc(-n5c6ccccc6c6ccccc65)cc5c6ccc7c(c8ccccc8n7-c7ccccc7)c6n(-c6ccccc6)c45)cc3)nc(-c3ccccc3)n2)cc1] 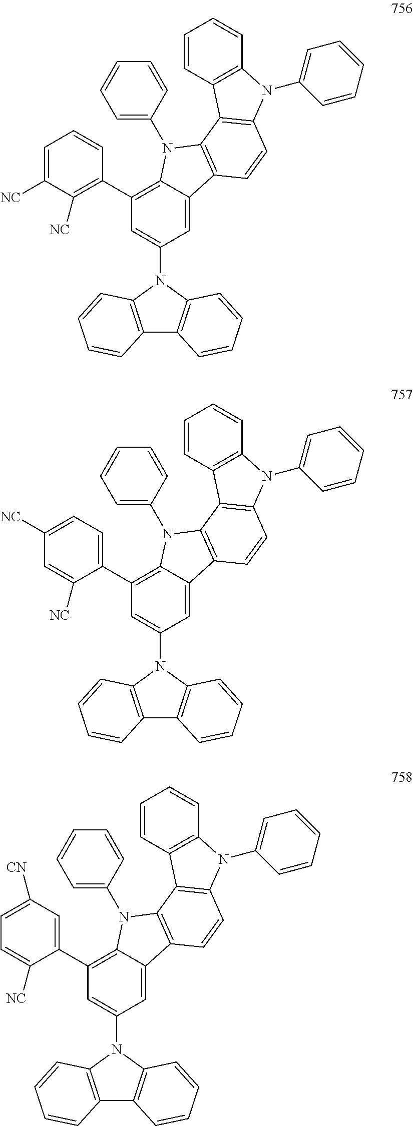 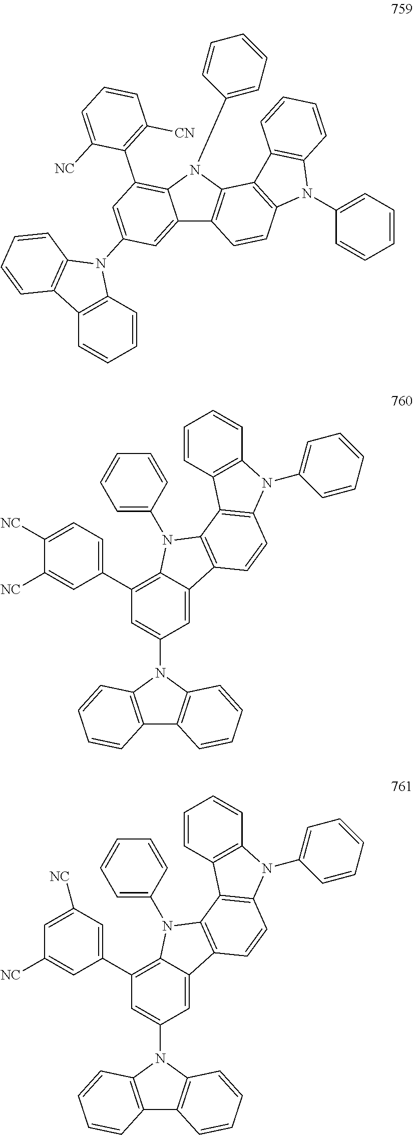 [SMILES: N#Cc1cccc(C#N)c1-c1cc(-n2c3ccccc3c3ccccc32)cc2c3ccc4c(c5ccccc5n4-c4ccccc4)c3n(-c3ccccc3)c12 ; N#Cc1ccc(-c2cc(-n3c4ccccc4c4ccccc43)cc3c4ccc5c(c6ccccc6n5-c5ccccc5)c4n(-c4ccccc4)c23)cc1C#N ; N#Cc1cc(C#N)cc(-c2cc(-n3c4ccccc4c4ccccc43)cc3c4ccc5c(c6ccccc6n5-c5ccccc5)c4n(-c4ccccc4)c23)c1] 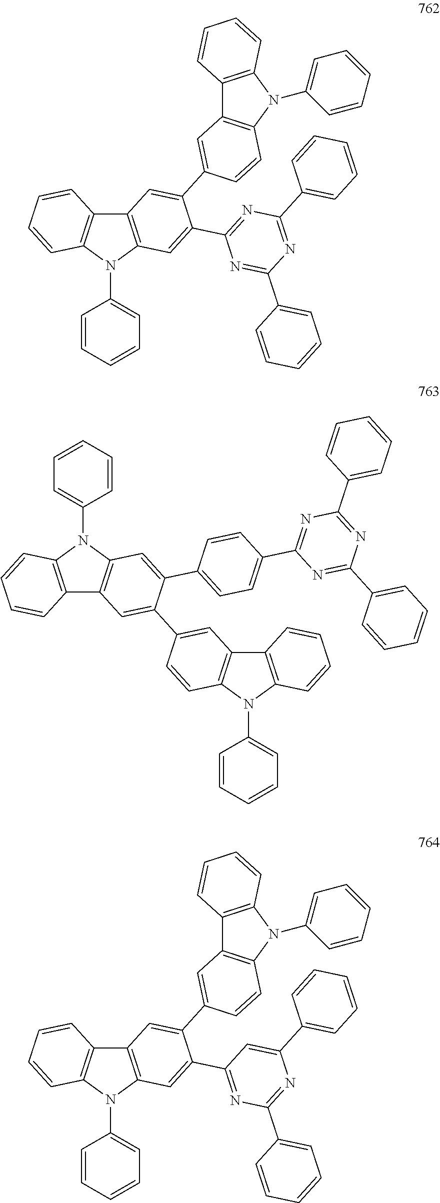 [SMILES: c1ccc(-c2nc(-c3ccccc3)nc(-c3cc4c(cc3-c3ccc5c(c3)c3ccccc3n5-c3ccccc3)c3ccccc3n4-c3ccccc3)n2)cc1 ; c1ccc(-c2nc(-c3ccccc3)nc(-c3ccc(-c4cc5c(cc4-c4ccc6c(c4)c4ccccc4n6-c4ccccc4)c4ccccc4n5-c4ccccc4)cc3)n2)cc1 ; c1ccc(-c2cc(-c3cc4c(cc3-c3ccc5c(c3)c3ccccc3n5-c3ccccc3)c3ccccc3n4-c3ccccc3)nc(-c3ccccc3)n2)cc1] 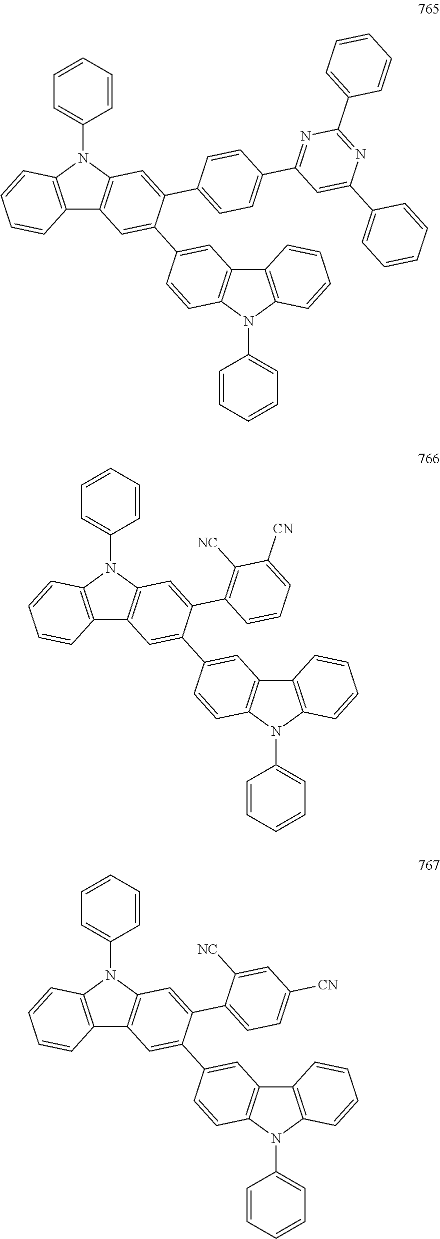 [SMILES: c1ccc(-c2cc(-c3ccc(-c4cc5c(cc4-c4ccc6c(c4)c4ccccc4n6-c4ccccc4)c4ccccc4n5-c4ccccc4)cc3)nc(-c3ccccc3)n2)cc1 ; N#Cc1cccc(-c2cc3c(cc2-c2ccc4c(c2)c2ccccc2n4-c2ccccc2)c2ccccc2n3-c2ccccc2)c1C#N ; N#Cc1ccc(-c2cc3c(cc2-c2ccc4c(c2)c2ccccc2n4-c2ccccc2)c2ccccc2n3-c2ccccc2)c(C#N)c1] 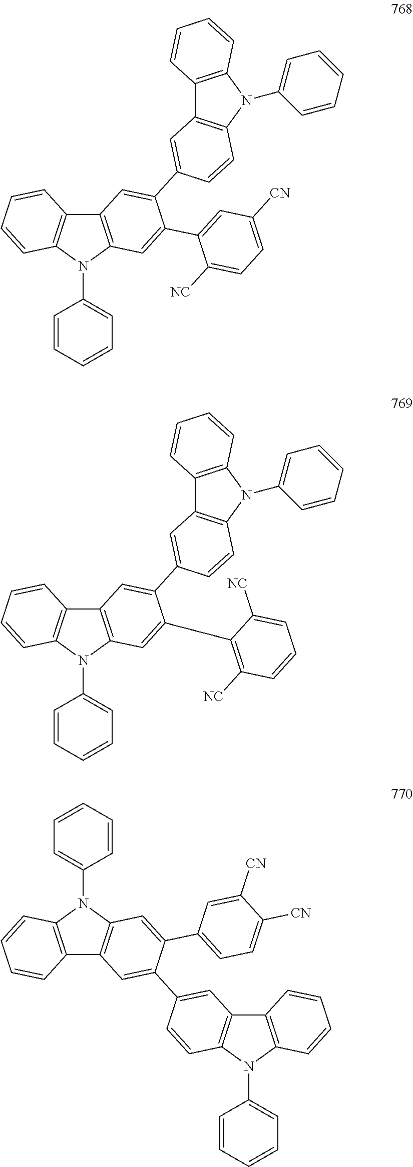 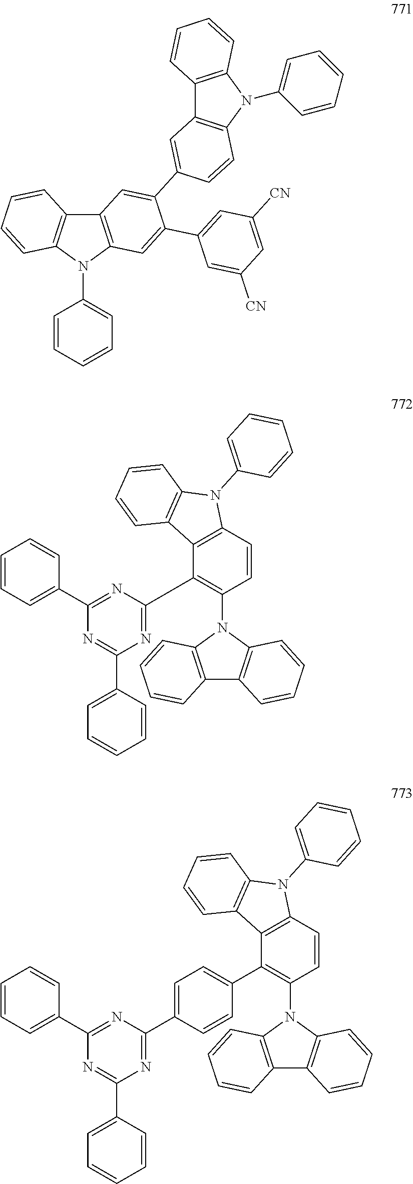 [SMILES: N#Cc1cc(C#N)cc(-c2cc3c(cc2-c2ccc4c(c2)c2ccccc2n4-c2ccccc2)c2ccccc2n3-c2ccccc2)c1 ; c1ccc(-c2nc(-c3ccccc3)nc(-c3c(-n4c5ccccc5c5ccccc54)ccc4c3c3ccccc3n4-c3ccccc3)n2)cc1 ; c1ccc(-c2nc(-c3ccccc3)nc(-c3ccc(-c4c(-n5c6ccccc6c6ccccc65)ccc5c4c4ccccc4n5-c4ccccc4)cc3)n2)cc1] 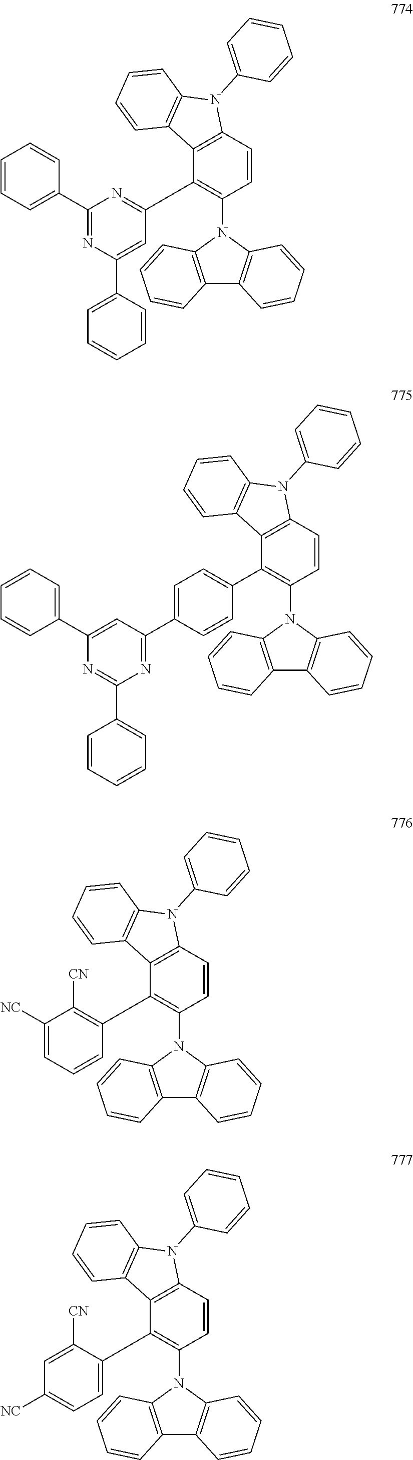 [SMILES: c1ccc(-c2cc(-c3c(-n4c5ccccc5c5ccccc54)ccc4c3c3ccccc3n4-c3ccccc3)nc(-c3ccccc3)n2)cc1 ; c1ccc(-c2cc(-c3ccc(-c4c(-n5c6ccccc6c6ccccc65)ccc5c4c4ccccc4n5-c4ccccc4)cc3)nc(-c3ccccc3)n2)cc1 ; N#Cc1cccc(-c2c(-n3c4ccccc4c4ccccc43)ccc3c2c2ccccc2n3-c2ccccc2)c1C#N ; N#Cc1ccc(-c2c(-n3c4ccccc4c4ccccc43)ccc3c2c2ccccc2n3-c2ccccc2)c(C#N)c1] 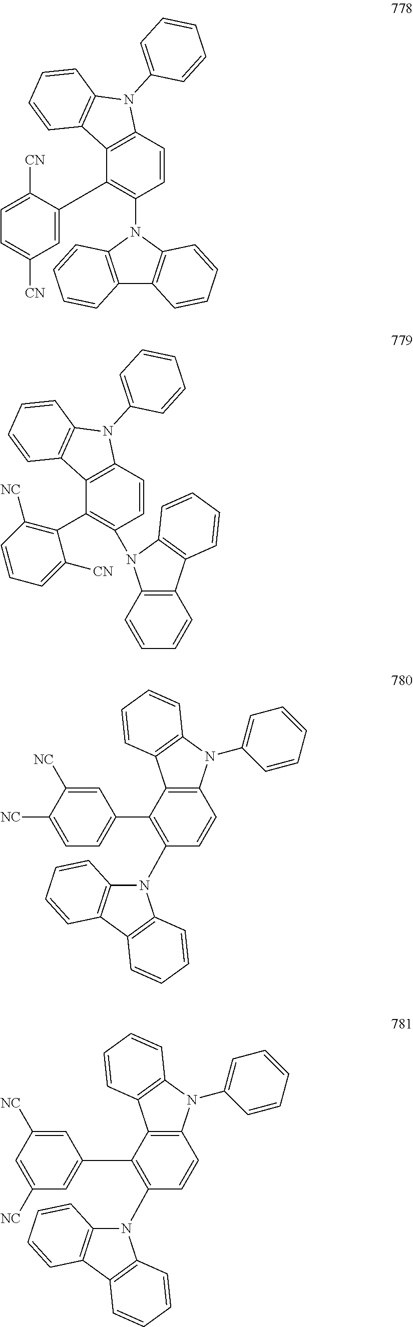 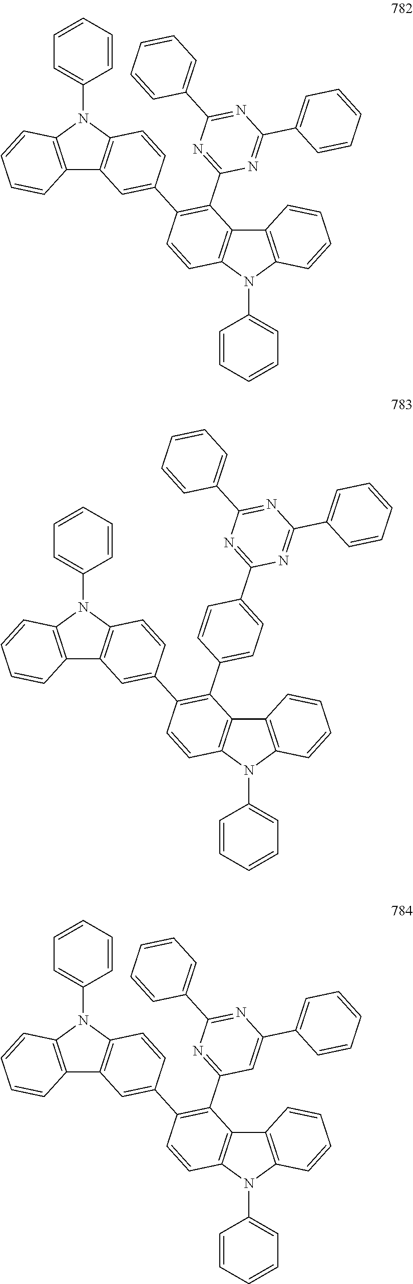 [SMILES: c1ccc(-c2nc(-c3ccccc3)nc(-c3c(-c4ccc5c(c4)c4ccccc4n5-c4ccccc4)ccc4c3c3ccccc3n4-c3ccccc3)n2)cc1 ; c1ccc(-c2nc(-c3ccccc3)nc(-c3ccc(-c4c(-c5ccc6c(c5)c5ccccc5n6-c5ccccc5)ccc5c4c4ccccc4n5-c4ccccc4)cc3)n2)cc1 ; c1ccc(-c2cc(-c3c(-c4ccc5c(c4)c4ccccc4n5-c4ccccc4)ccc4c3c3ccccc3n4-c3ccccc3)nc(-c3ccccc3)n2)cc1] 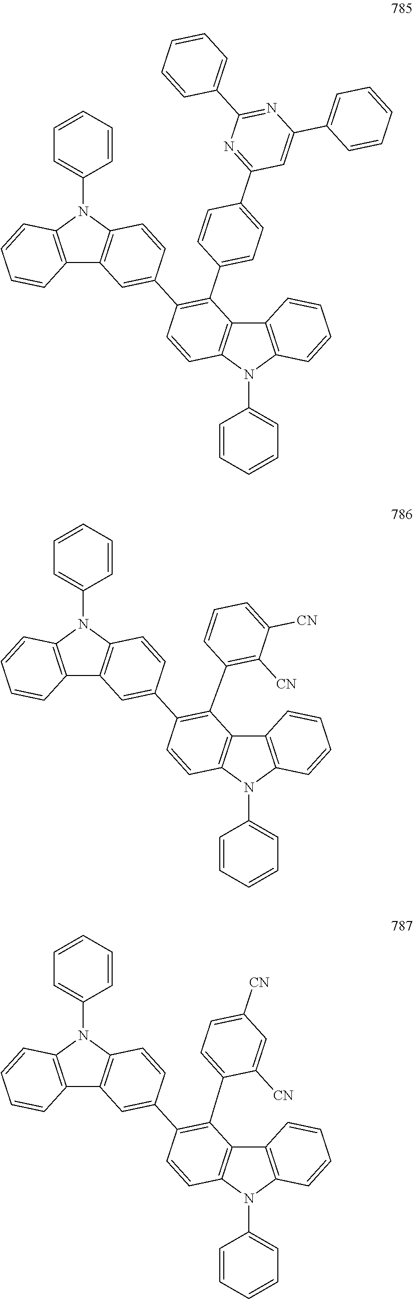 [SMILES: c1ccc(-c2cc(-c3ccc(-c4c(-c5ccc6c(c5)c5ccccc5n6-c5ccccc5)ccc5c4c4ccccc4n5-c4ccccc4)cc3)nc(-c3ccccc3)n2)cc1 ; N#Cc1cccc(-c2c(-c3ccc4c(c3)c3ccccc3n4-c3ccccc3)ccc3c2c2ccccc2n3-c2ccccc2)c1C#N ; N#Cc1ccc(-c2c(-c3ccc4c(c3)c3ccccc3n4-c3ccccc3)ccc3c2c2ccccc2n3-c2ccccc2)c(C#N)c1] 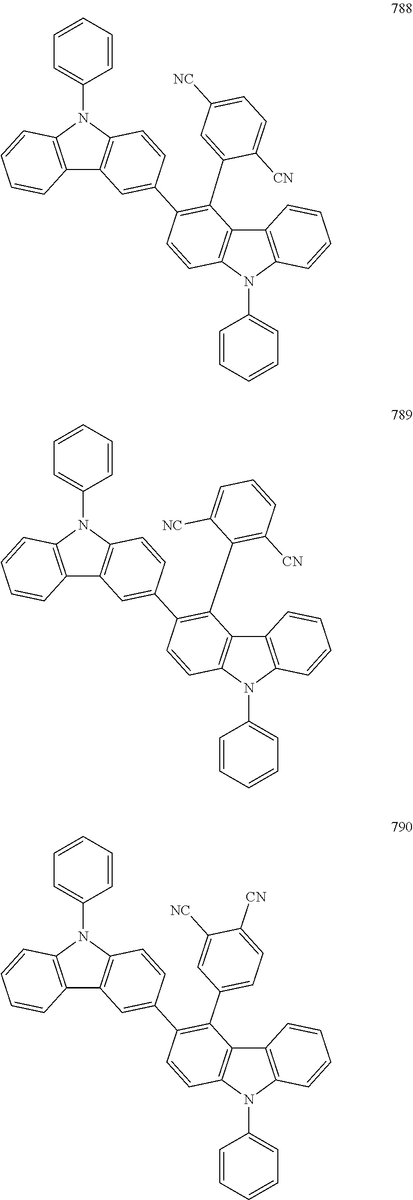 [SMILES: N#Cc1ccc(C#N)c(-c2c(-c3ccc4c(c3)c3ccccc3n4-c3ccccc3)ccc3c2c2ccccc2n3-c2ccccc2)c1 ; N#Cc1cccc(C#N)c1-c1c(-c2ccc3c(c2)c2ccccc2n3-c2ccccc2)ccc2c1c1ccccc1n2-c1ccccc1 ; N#Cc1ccc(-c2c(-c3ccc4c(c3)c3ccccc3n4-c3ccccc3)ccc3c2c2ccccc2n3-c2ccccc2)cc1C#N] 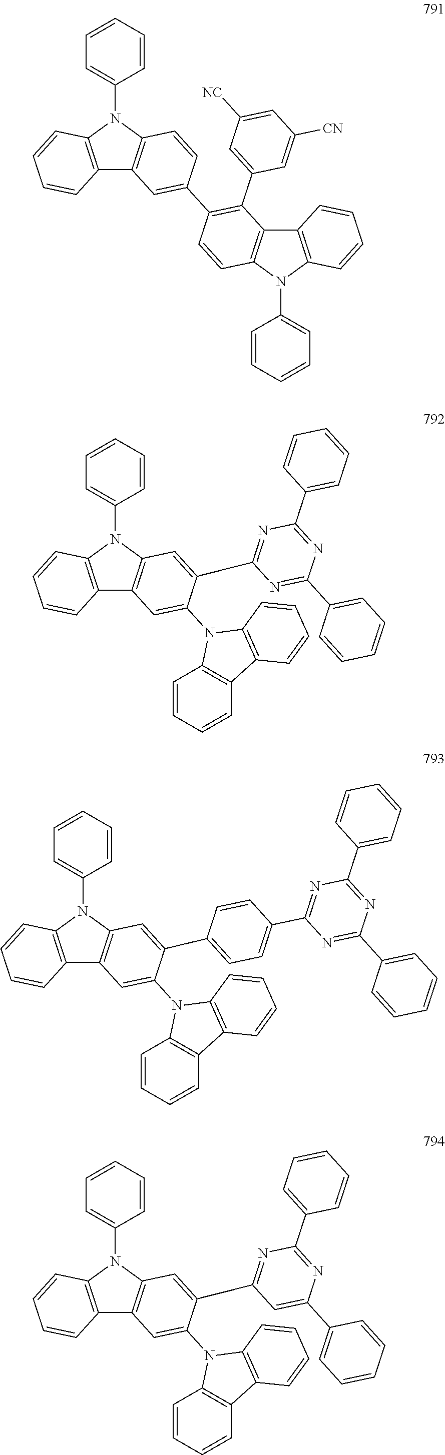 [SMILES: N#Cc1cc(C#N)cc(-c2c(-c3ccc4c(c3)c3ccccc3n4-c3ccccc3)ccc3c2c2ccccc2n3-c2ccccc2)c1 ; c1ccc(-c2nc(-c3ccccc3)nc(-c3cc4c(cc3-n3c5ccccc5c5ccccc53)c3ccccc3n4-c3ccccc3)n2)cc1 ; c1ccc(-c2nc(-c3ccccc3)nc(-c3ccc(-c4cc5c(cc4-n4c6ccccc6c6ccccc64)c4ccccc4n5-c4ccccc4)cc3)n2)cc1 ; c1ccc(-c2cc(-c3cc4c(cc3-n3c5ccccc5c5ccccc53)c3ccccc3n4-c3ccccc3)nc(-c3ccccc3)n2)cc1] 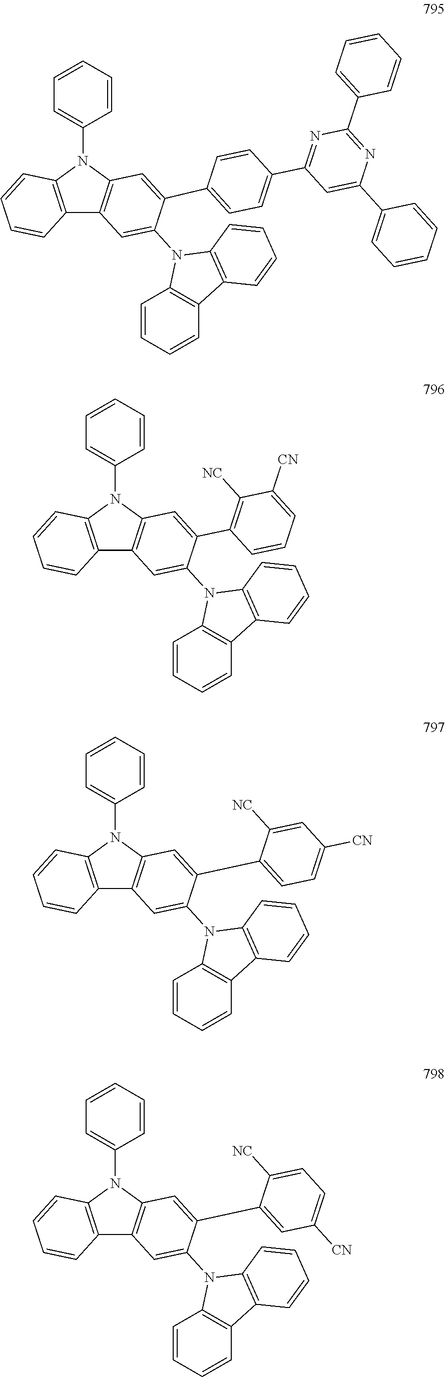 [SMILES: c1ccc(-c2cc(-c3ccc(-c4cc5c(cc4-n4c6ccccc6c6ccccc64)c4ccccc4n5-c4ccccc4)cc3)nc(-c3ccccc3)n2)cc1 ; N#Cc1cccc(-c2cc3c(cc2-n2c4ccccc4c4ccccc42)c2ccccc2n3-c2ccccc2)c1C#N ; N#Cc1ccc(-c2cc3c(cc2-n2c4ccccc4c4ccccc42)c2ccccc2n3-c2ccccc2)c(C#N)c1 ; N#Cc1ccc(C#N)c(-c2cc3c(cc2-n2c4ccccc4c4ccccc42)c2ccccc2n3-c2ccccc2)c1] 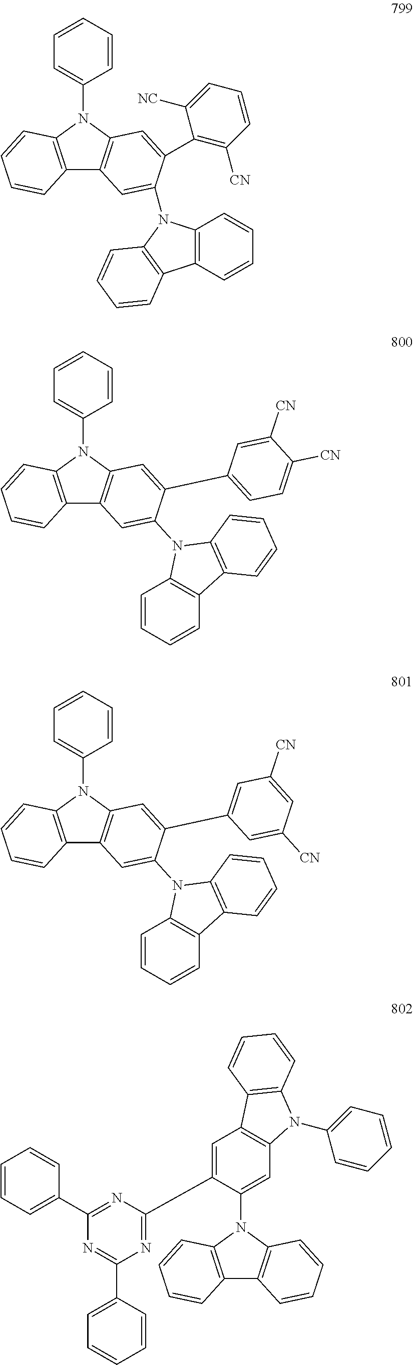 [SMILES: N#Cc1cccc(C#N)c1-c1cc2c(cc1-n1c3ccccc3c3ccccc31)c1ccccc1n2-c1ccccc1 ; N#Cc1ccc(-c2cc3c(cc2-n2c4ccccc4c4ccccc42)c2ccccc2n3-c2ccccc2)cc1C#N ; N#Cc1cc(C#N)cc(-c2cc3c(cc2-n2c4ccccc4c4ccccc42)c2ccccc2n3-c2ccccc2)c1 ; c1ccc(-c2nc(-c3ccccc3)nc(-c3cc4c5ccccc5n(-c5ccccc5)c4cc3-n3c4ccccc4c4ccccc43)n2)cc1] 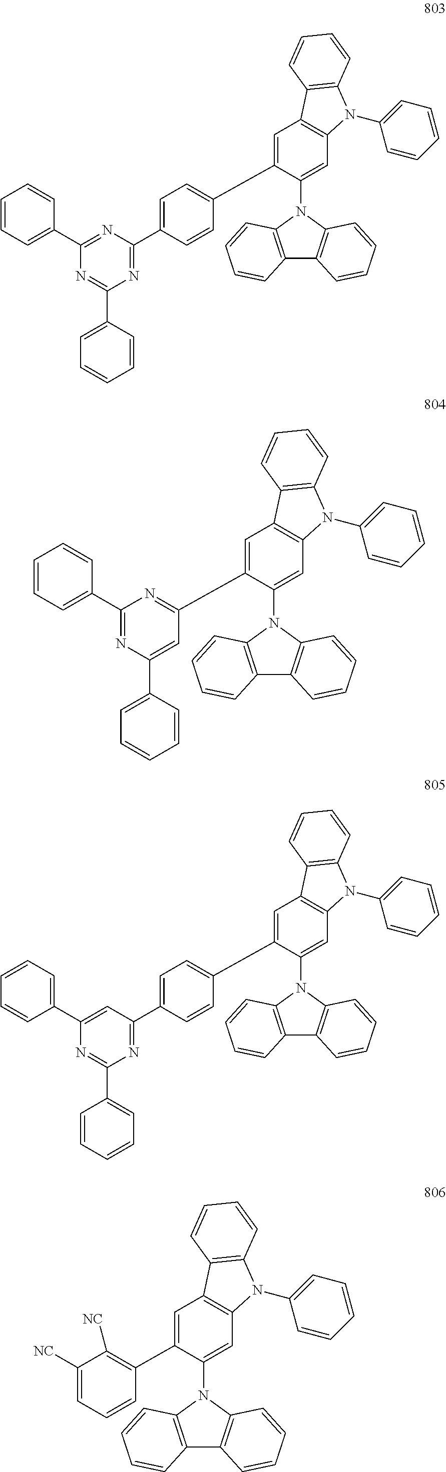 [SMILES: c1ccc(-c2nc(-c3ccccc3)nc(-c3ccc(-c4cc5c6ccccc6n(-c6ccccc6)c5cc4-n4c5ccccc5c5ccccc54)cc3)n2)cc1 ; c1ccc(-c2cc(-c3cc4c5ccccc5n(-c5ccccc5)c4cc3-n3c4ccccc4c4ccccc43)nc(-c3ccccc3)n2)cc1 ; c1ccc(-c2cc(-c3ccc(-c4cc5c6ccccc6n(-c6ccccc6)c5cc4-n4c5ccccc5c5ccccc54)cc3)nc(-c3ccccc3)n2)cc1 ; N#Cc1cccc(-c2cc3c4ccccc4n(-c4ccccc4)c3cc2-n2c3ccccc3c3ccccc32)c1C#N] 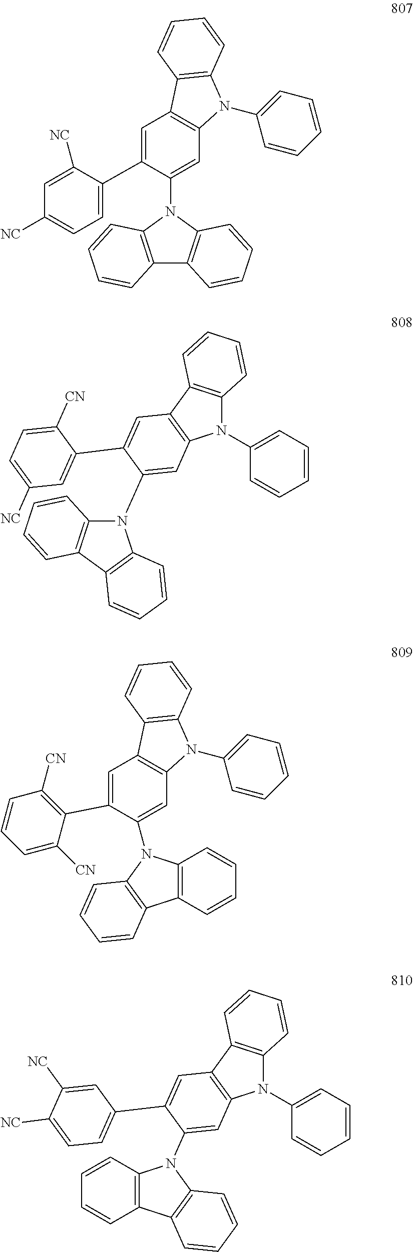 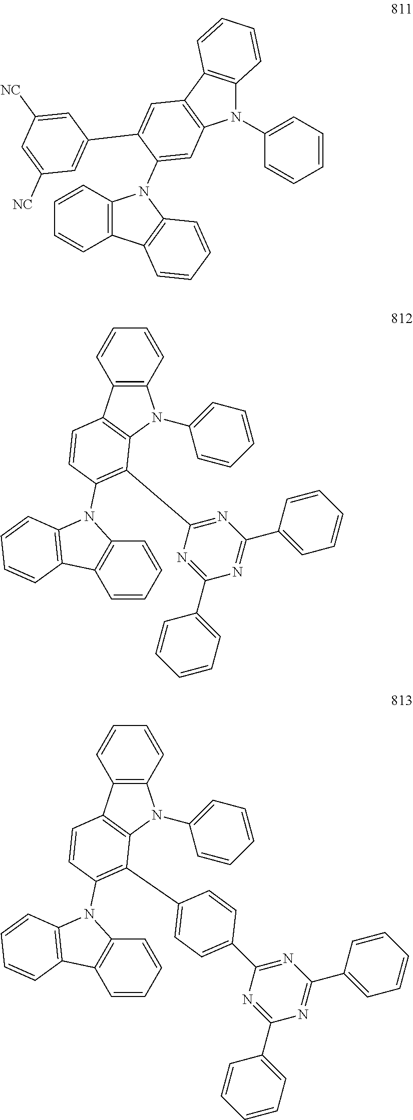 [SMILES: N#Cc1cc(C#N)cc(-c2cc3c4ccccc4n(-c4ccccc4)c3cc2-n2c3ccccc3c3ccccc32)c1 ; c1ccc(-c2nc(-c3ccccc3)nc(-c3c(-n4c5ccccc5c5ccccc54)ccc4c5ccccc5n(-c5ccccc5)c34)n2)cc1 ; c1ccc(-c2nc(-c3ccccc3)nc(-c3ccc(-c4c(-n5c6ccccc6c6ccccc65)ccc5c6ccccc6n(-c6ccccc6)c45)cc3)n2)cc1] 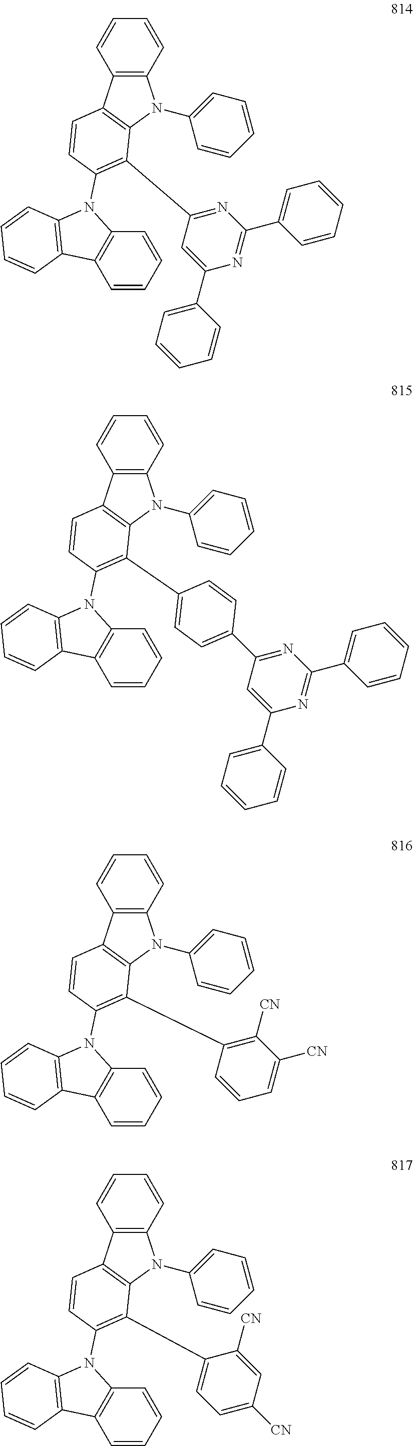 [SMILES: c1ccc(-c2cc(-c3c(-n4c5ccccc5c5ccccc54)ccc4c5ccccc5n(-c5ccccc5)c34)nc(-c3ccccc3)n2)cc1 ; c1ccc(-c2cc(-c3ccc(-c4c(-n5c6ccccc6c6ccccc65)ccc5c6ccccc6n(-c6ccccc6)c45)cc3)nc(-c3ccccc3)n2)cc1 ; N#Cc1cccc(-c2c(-n3c4ccccc4c4ccccc43)ccc3c4ccccc4n(-c4ccccc4)c23)c1C#N ; N#Cc1ccc(-c2c(-n3c4ccccc4c4ccccc43)ccc3c4ccccc4n(-c4ccccc4)c23)c(C#N)c1] 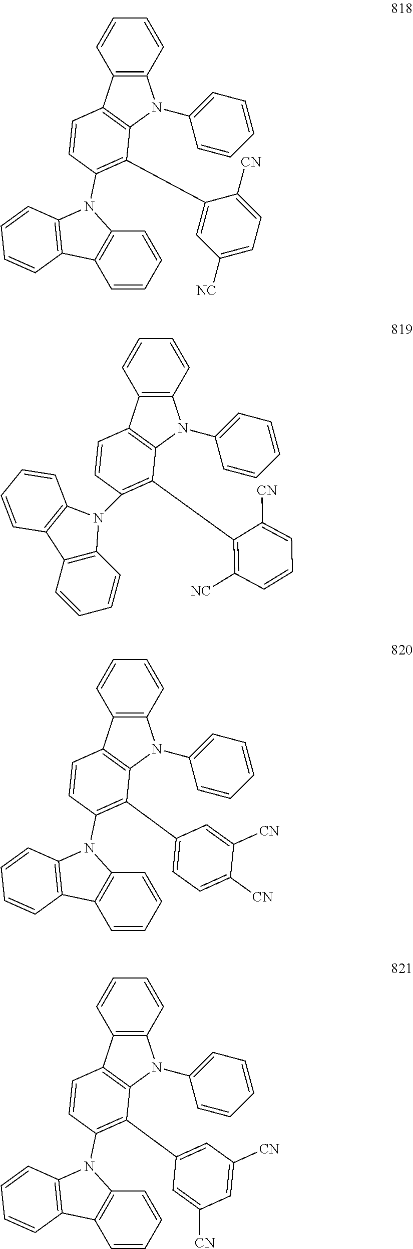 [SMILES: N#Cc1ccc(C#N)c(-c2c(-n3c4ccccc4c4ccccc43)ccc3c4ccccc4n(-c4ccccc4)c23)c1 ; N#Cc1cccc(C#N)c1-c1c(-n2c3ccccc3c3ccccc32)ccc2c3ccccc3n(-c3ccccc3)c12 ; N#Cc1ccc(-c2c(-n3c4ccccc4c4ccccc43)ccc3c4ccccc4n(-c4ccccc4)c23)cc1C#N ; N#Cc1cc(C#N)cc(-c2c(-n3c4ccccc4c4ccccc43)ccc3c4ccccc4n(-c4ccccc4)c23)c1] 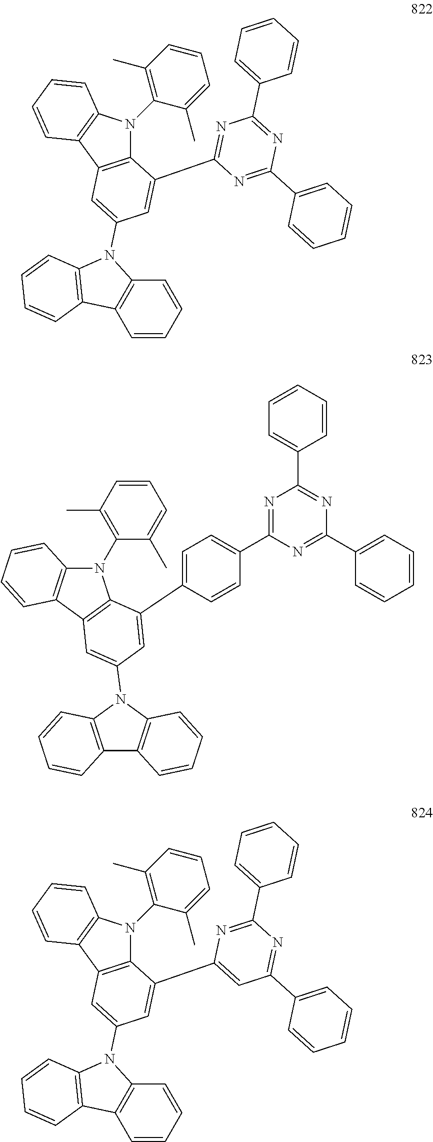 [SMILES: Cc1cccc(C)c1-n1c2ccccc2c2cc(-n3c4ccccc4c4ccccc43)cc(-c3nc(-c4ccccc4)nc(-c4ccccc4)n3)c21 ; Cc1cccc(C)c1-n1c2ccccc2c2cc(-n3c4ccccc4c4ccccc43)cc(-c3ccc(-c4nc(-c5ccccc5)nc(-c5ccccc5)n4)cc3)c21 ; Cc1cccc(C)c1-n1c2ccccc2c2cc(-n3c4ccccc4c4ccccc43)cc(-c3cc(-c4ccccc4)nc(-c4ccccc4)n3)c21] 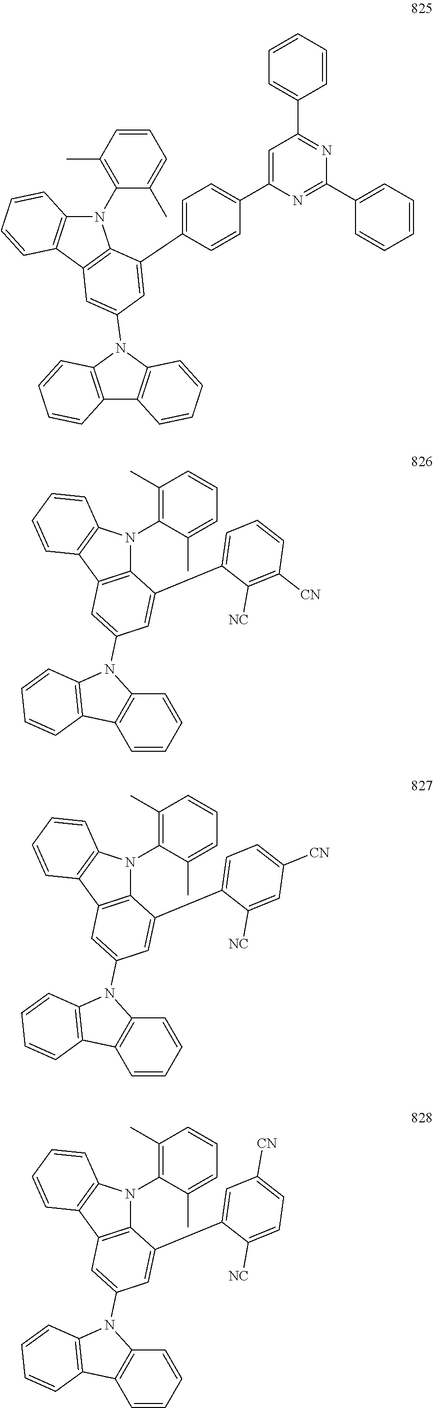 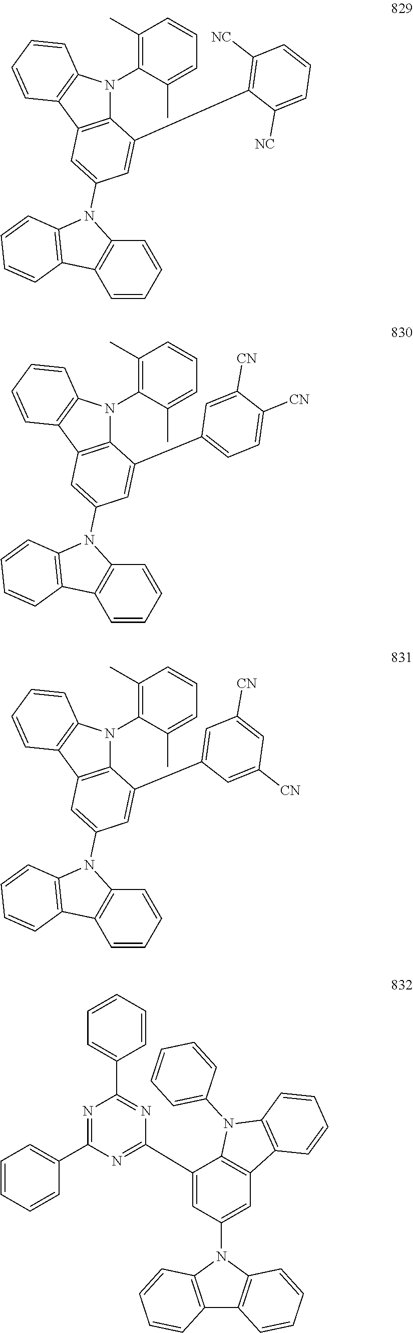 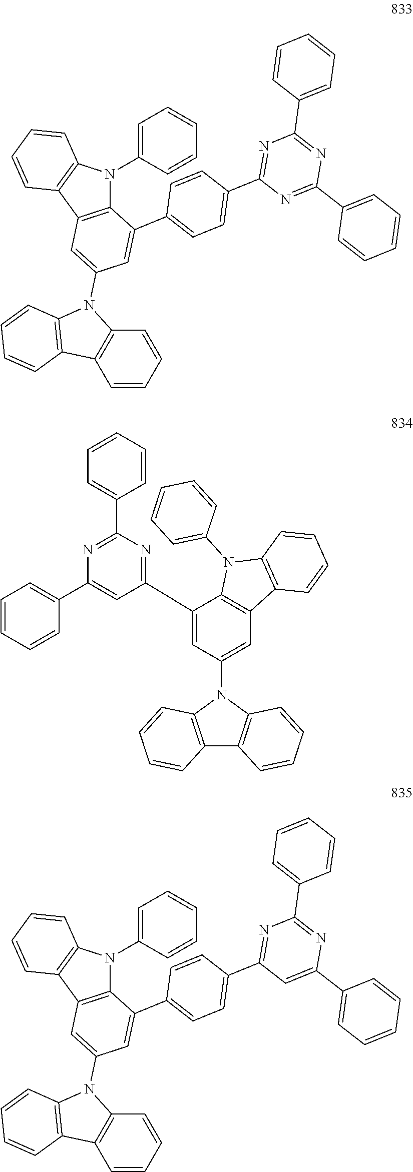 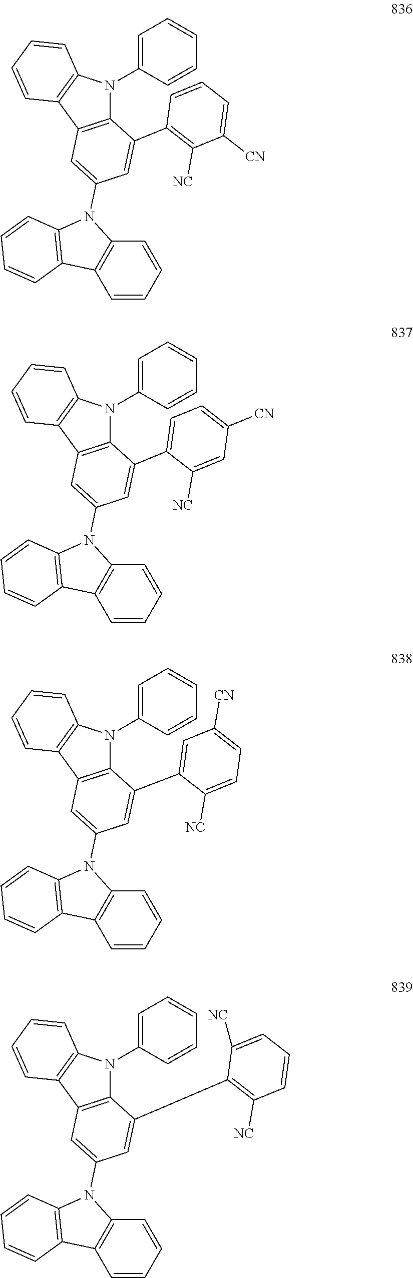 [SMILES: N#Cc1cccc(-c2cc(-n3c4ccccc4c4ccccc43)cc3c4ccccc4n(-c4ccccc4)c23)c1C#N ; N#Cc1ccc(-c2cc(-n3c4ccccc4c4ccccc43)cc3c4ccccc4n(-c4ccccc4)c23)c(C#N)c1 ; N#Cc1ccc(C#N)c(-c2cc(-n3c4ccccc4c4ccccc43)cc3c4ccccc4n(-c4ccccc4)c23)c1 ; N#Cc1cccc(C#N)c1-c1cc(-n2c3ccccc3c3ccccc32)cc2c3ccccc3n(-c3ccccc3)c12] 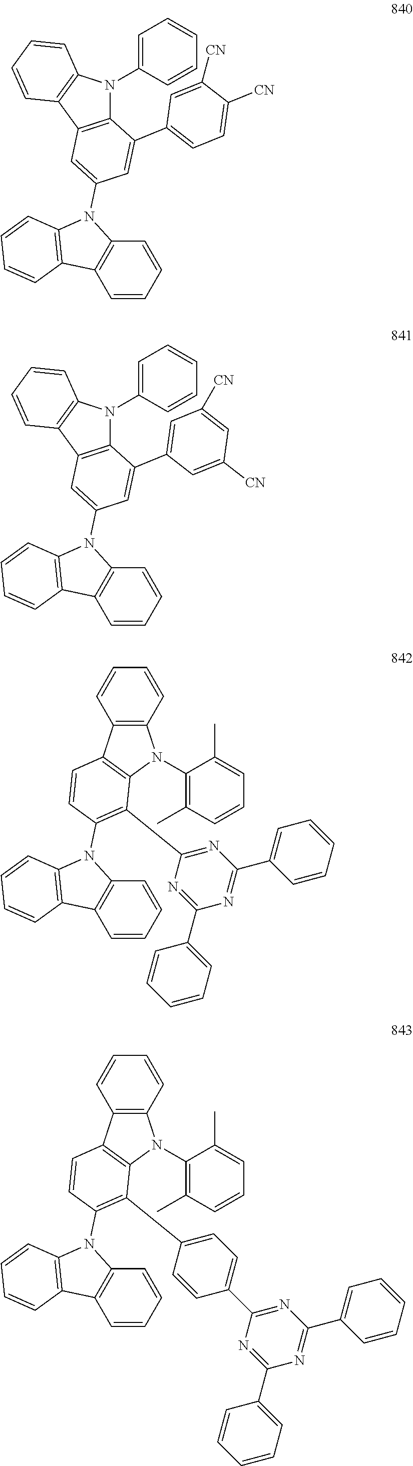 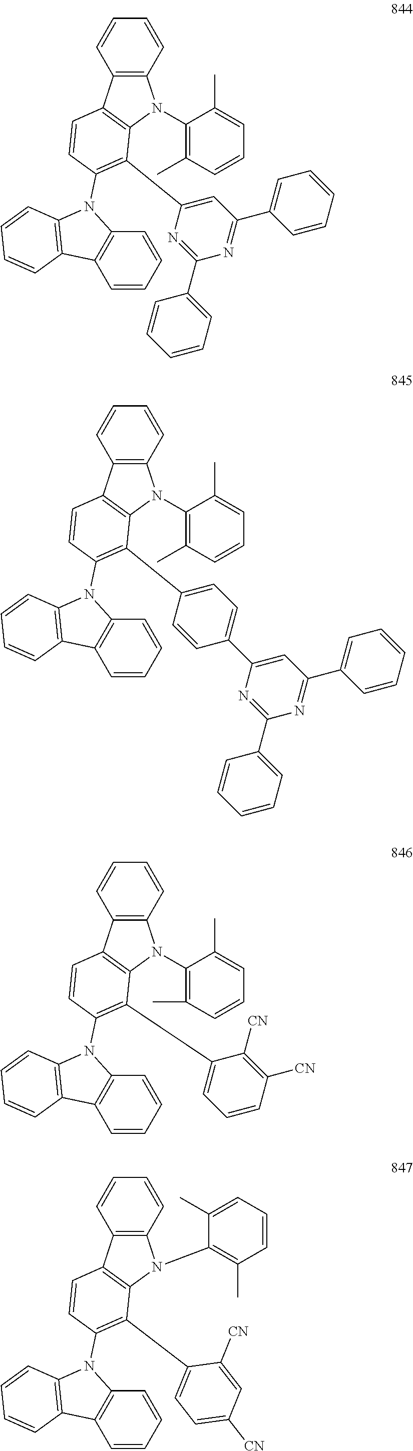 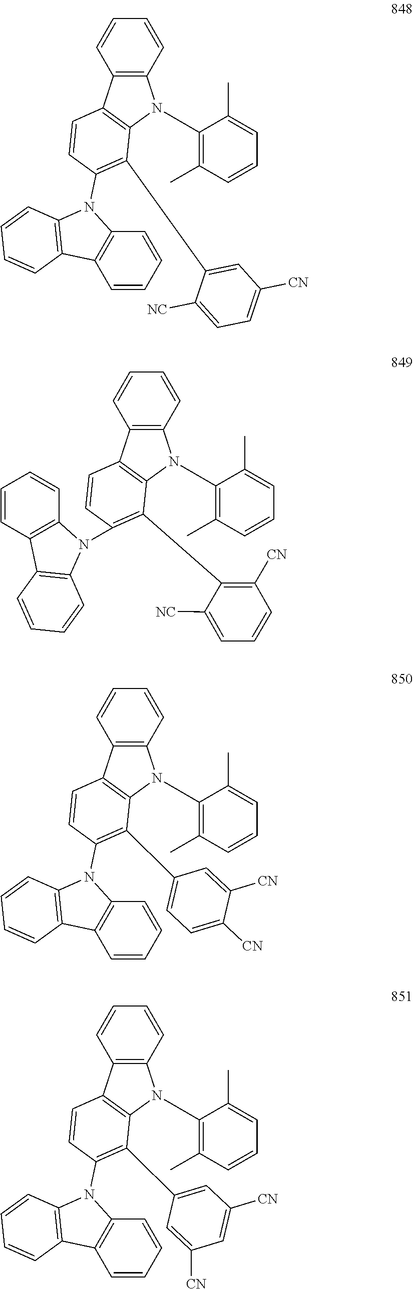 [SMILES: Cc1cccc(C)c1-n1c2ccccc2c2ccc(-n3c4ccccc4c4ccccc43)c(-c3cc(C#N)ccc3C#N)c21 ; Cc1cccc(C)c1-n1c2ccccc2c2ccc(-n3c4ccccc4c4ccccc43)c(-c3c(C#N)cccc3C#N)c21 ; Cc1cccc(C)c1-n1c2ccccc2c2ccc(-n3c4ccccc4c4ccccc43)c(-c3ccc(C#N)c(C#N)c3)c21 ; Cc1cccc(C)c1-n1c2ccccc2c2ccc(-n3c4ccccc4c4ccccc43)c(-c3cc(C#N)cc(C#N)c3)c21] 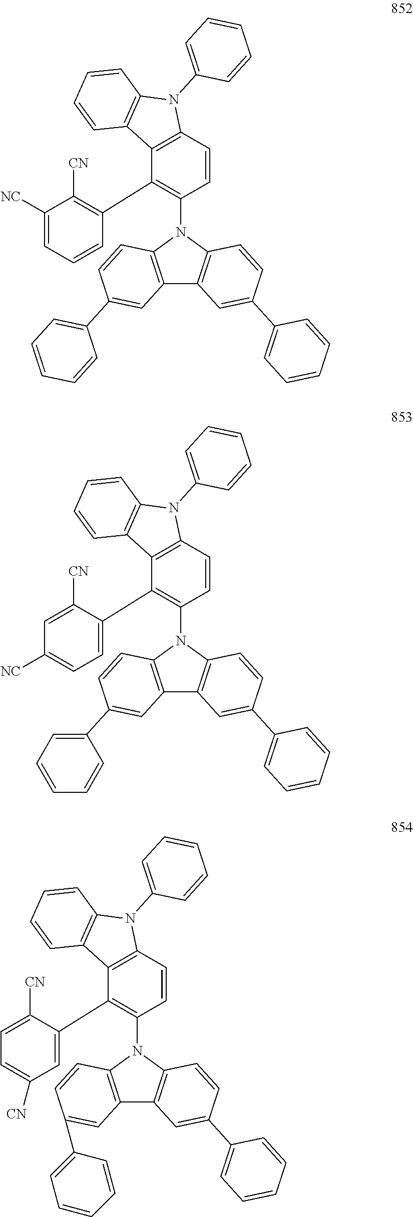 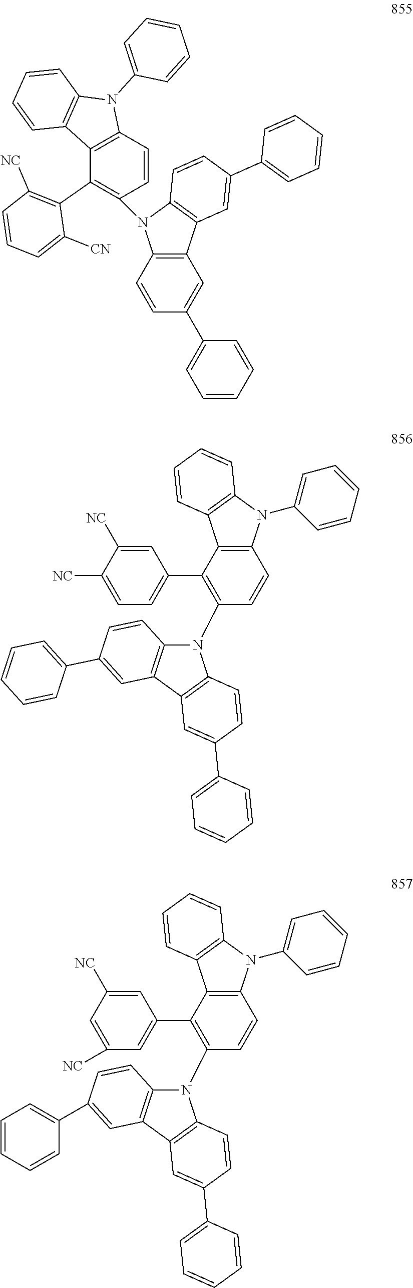 [SMILES: N#Cc1cccc(C#N)c1-c1c(-n2c3ccc(-c4ccccc4)cc3c3cc(-c4ccccc4)ccc32)ccc2c1c1ccccc1n2-c1ccccc1 ; N#Cc1ccc(-c2c(-n3c4ccc(-c5ccccc5)cc4c4cc(-c5ccccc5)ccc43)ccc3c2c2ccccc2n3-c2ccccc2)cc1C#N ; N#Cc1cc(C#N)cc(-c2c(-n3c4ccc(-c5ccccc5)cc4c4cc(-c5ccccc5)ccc43)ccc3c2c2ccccc2n3-c2ccccc2)c1] 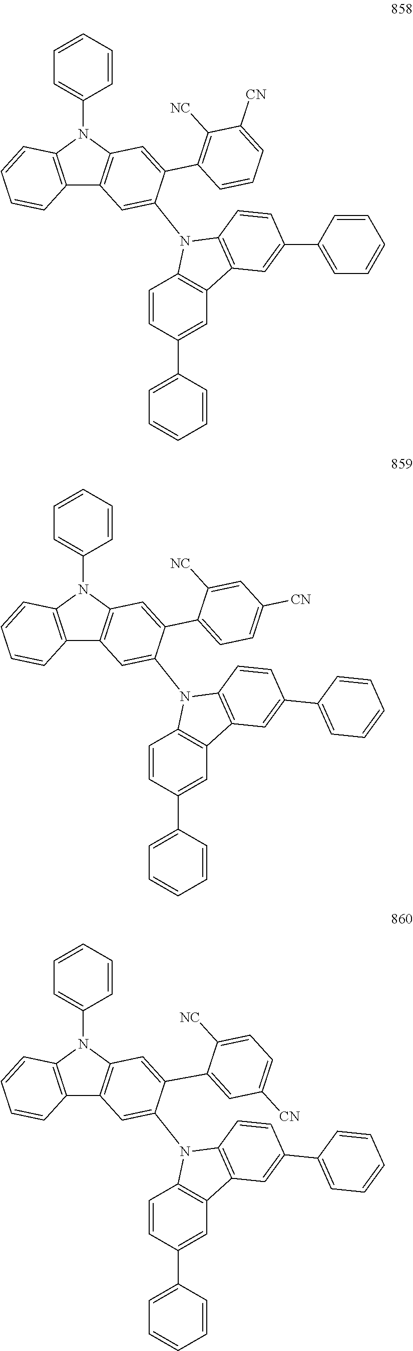 [SMILES: N#Cc1cccc(-c2cc3c(cc2-n2c4ccc(-c5ccccc5)cc4c4cc(-c5ccccc5)ccc42)c2ccccc2n3-c2ccccc2)c1C#N ; N#Cc1ccc(-c2cc3c(cc2-n2c4ccc(-c5ccccc5)cc4c4cc(-c5ccccc5)ccc42)c2ccccc2n3-c2ccccc2)c(C#N)c1 ; N#Cc1ccc(C#N)c(-c2cc3c(cc2-n2c4ccc(-c5ccccc5)cc4c4cc(-c5ccccc5)ccc42)c2ccccc2n3-c2ccccc2)c1] 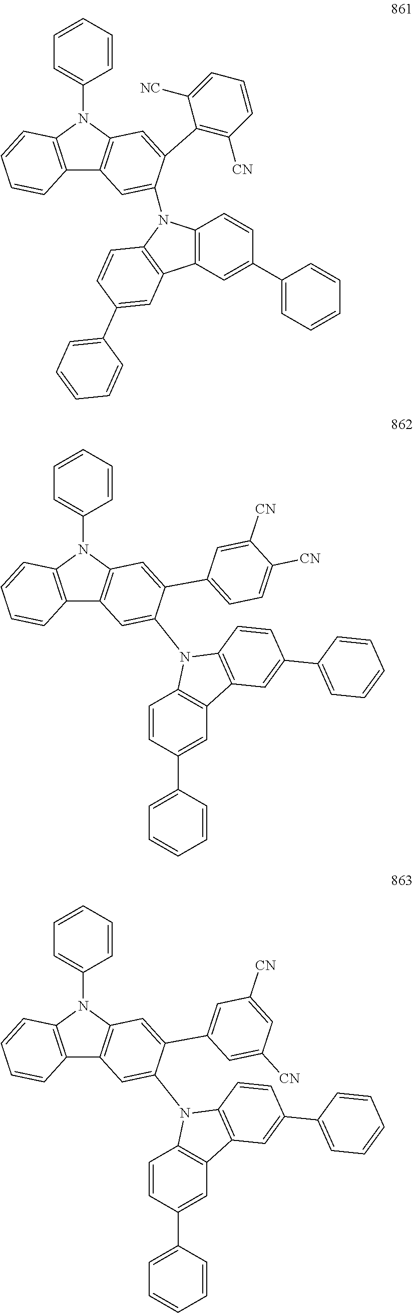 [SMILES: N#Cc1cccc(C#N)c1-c1cc2c(cc1-n1c3ccc(-c4ccccc4)cc3c3cc(-c4ccccc4)ccc31)c1ccccc1n2-c1ccccc1 ; N#Cc1ccc(-c2cc3c(cc2-n2c4ccc(-c5ccccc5)cc4c4cc(-c5ccccc5)ccc42)c2ccccc2n3-c2ccccc2)cc1C#N ; N#Cc1cc(C#N)cc(-c2cc3c(cc2-n2c4ccc(-c5ccccc5)cc4c4cc(-c5ccccc5)ccc42)c2ccccc2n3-c2ccccc2)c1] 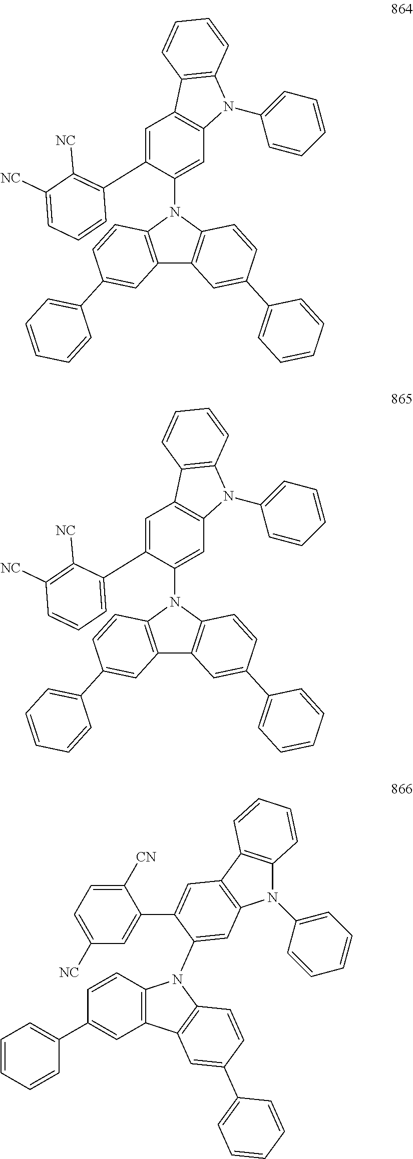 [SMILES: N#Cc1cccc(-c2cc3c4ccccc4n(-c4ccccc4)c3cc2-n2c3ccc(-c4ccccc4)cc3c3cc(-c4ccccc4)ccc32)c1C#N ; N#Cc1cccc(-c2cc3c4ccccc4n(-c4ccccc4)c3cc2-n2c3ccc(-c4ccccc4)cc3c3cc(-c4ccccc4)ccc32)c1C#N ; N#Cc1ccc(C#N)c(-c2cc3c4ccccc4n(-c4ccccc4)c3cc2-n2c3ccc(-c4ccccc4)cc3c3cc(-c4ccccc4)ccc32)c1] 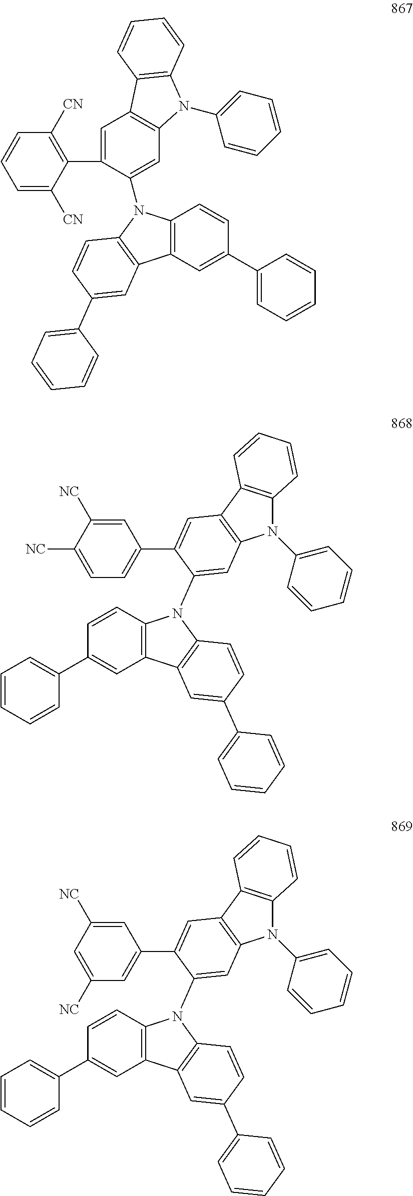 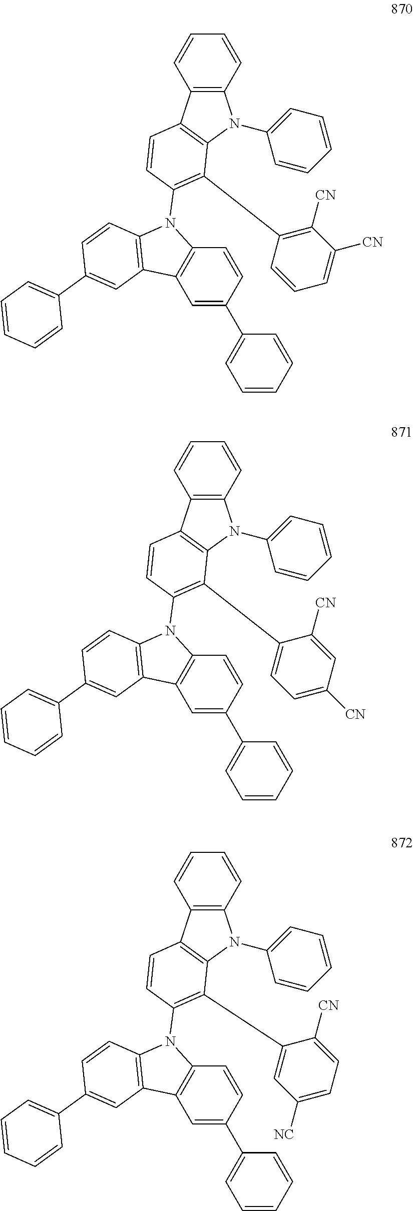 [SMILES: N#Cc1cccc(-c2c(-n3c4ccc(-c5ccccc5)cc4c4cc(-c5ccccc5)ccc43)ccc3c4ccccc4n(-c4ccccc4)c23)c1C#N ; N#Cc1ccc(-c2c(-n3c4ccc(-c5ccccc5)cc4c4cc(-c5ccccc5)ccc43)ccc3c4ccccc4n(-c4ccccc4)c23)c(C#N)c1 ; N#Cc1ccc(C#N)c(-c2c(-n3c4ccc(-c5ccccc5)cc4c4cc(-c5ccccc5)ccc43)ccc3c4ccccc4n(-c4ccccc4)c23)c1] 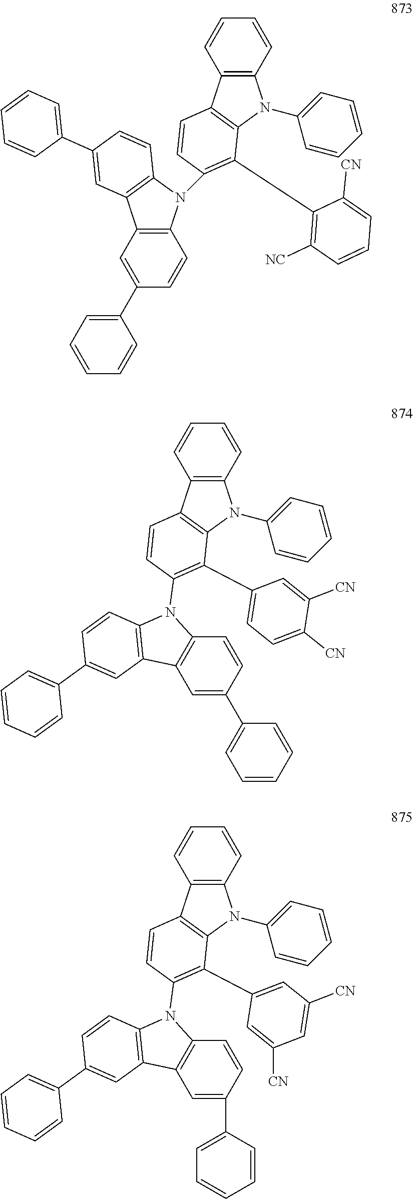 [SMILES: N#Cc1cccc(C#N)c1-c1c(-n2c3ccc(-c4ccccc4)cc3c3cc(-c4ccccc4)ccc32)ccc2c3ccccc3n(-c3ccccc3)c12 ; N#Cc1ccc(-c2c(-n3c4ccc(-c5ccccc5)cc4c4cc(-c5ccccc5)ccc43)ccc3c4ccccc4n(-c4ccccc4)c23)cc1C#N ; N#Cc1cc(C#N)cc(-c2c(-n3c4ccc(-c5ccccc5)cc4c4cc(-c5ccccc5)ccc43)ccc3c4ccccc4n(-c4ccccc4)c23)c1] 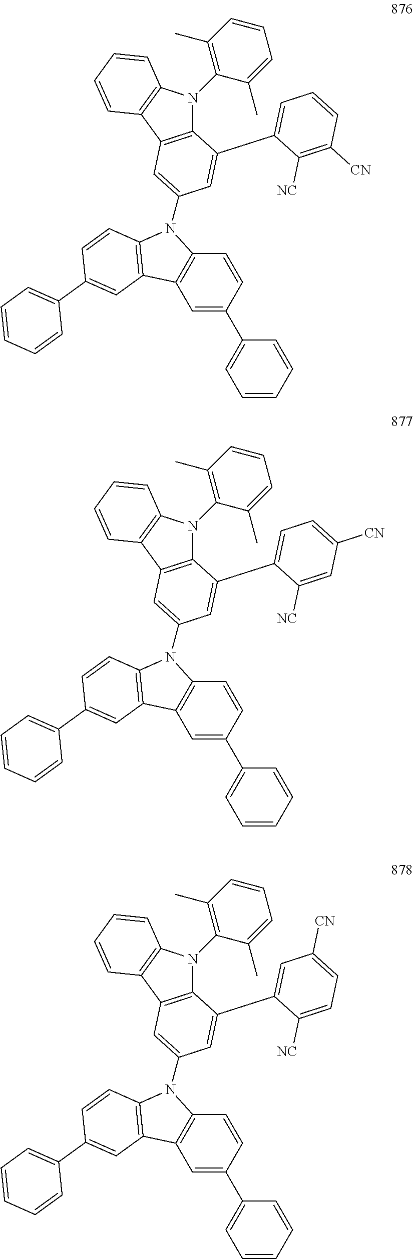 [SMILES: Cc1cccc(C)c1-n1c2ccccc2c2cc(-n3c4ccc(-c5ccccc5)cc4c4cc(-c5ccccc5)ccc43)cc(-c3cccc(C#N)c3C#N)c21 ; Cc1cccc(C)c1-n1c2ccccc2c2cc(-n3c4ccc(-c5ccccc5)cc4c4cc(-c5ccccc5)ccc43)cc(-c3ccc(C#N)cc3C#N)c21 ; Cc1cccc(C)c1-n1c2ccccc2c2cc(-n3c4ccc(-c5ccccc5)cc4c4cc(-c5ccccc5)ccc43)cc(-c3cc(C#N)ccc3C#N)c21] 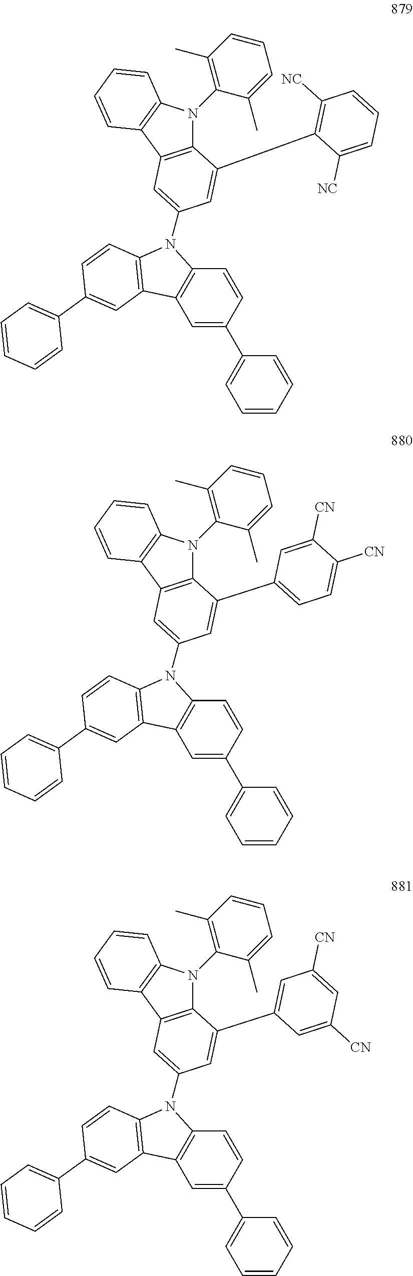 [SMILES: Cc1cccc(C)c1-n1c2ccccc2c2cc(-n3c4ccc(-c5ccccc5)cc4c4cc(-c5ccccc5)ccc43)cc(-c3c(C#N)cccc3C#N)c21 ; Cc1cccc(C)c1-n1c2ccccc2c2cc(-n3c4ccc(-c5ccccc5)cc4c4cc(-c5ccccc5)ccc43)cc(-c3ccc(C#N)c(C#N)c3)c21 ; Cc1cccc(C)c1-n1c2ccccc2c2cc(-n3c4ccc(-c5ccccc5)cc4c4cc(-c5ccccc5)ccc43)cc(-c3cc(C#N)cc(C#N)c3)c21] 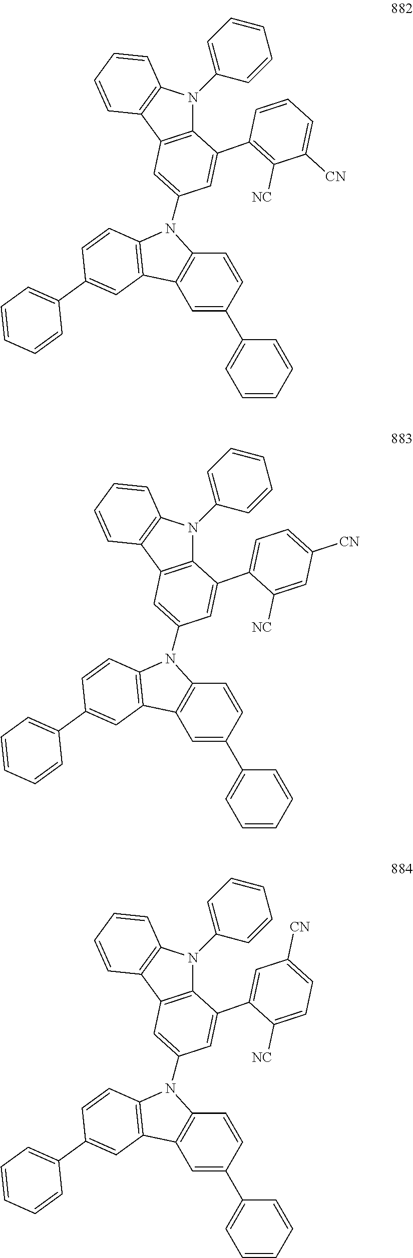 [SMILES: N#Cc1cccc(-c2cc(-n3c4ccc(-c5ccccc5)cc4c4cc(-c5ccccc5)ccc43)cc3c4ccccc4n(-c4ccccc4)c23)c1C#N ; N#Cc1ccc(-c2cc(-n3c4ccc(-c5ccccc5)cc4c4cc(-c5ccccc5)ccc43)cc3c4ccccc4n(-c4ccccc4)c23)c(C#N)c1 ; N#Cc1ccc(C#N)c(-c2cc(-n3c4ccc(-c5ccccc5)cc4c4cc(-c5ccccc5)ccc43)cc3c4ccccc4n(-c4ccccc4)c23)c1] 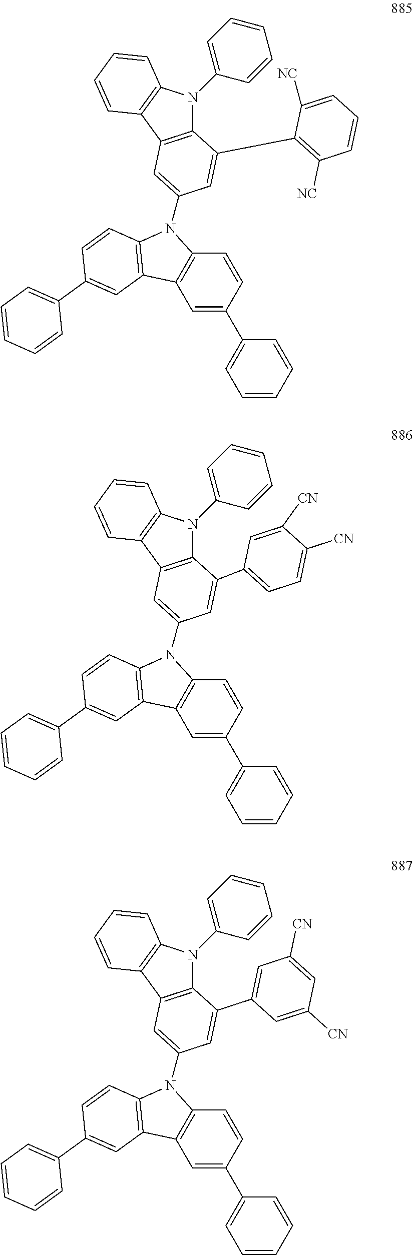 [SMILES: N#Cc1cccc(C#N)c1-c1cc(-n2c3ccc(-c4ccccc4)cc3c3cc(-c4ccccc4)ccc32)cc2c3ccccc3n(-c3ccccc3)c12 ; N#Cc1ccc(-c2cc(-n3c4ccc(-c5ccccc5)cc4c4cc(-c5ccccc5)ccc43)cc3c4ccccc4n(-c4ccccc4)c23)cc1C#N ; N#Cc1cc(C#N)cc(-c2cc(-n3c4ccc(-c5ccccc5)cc4c4cc(-c5ccccc5)ccc43)cc3c4ccccc4n(-c4ccccc4)c23)c1] 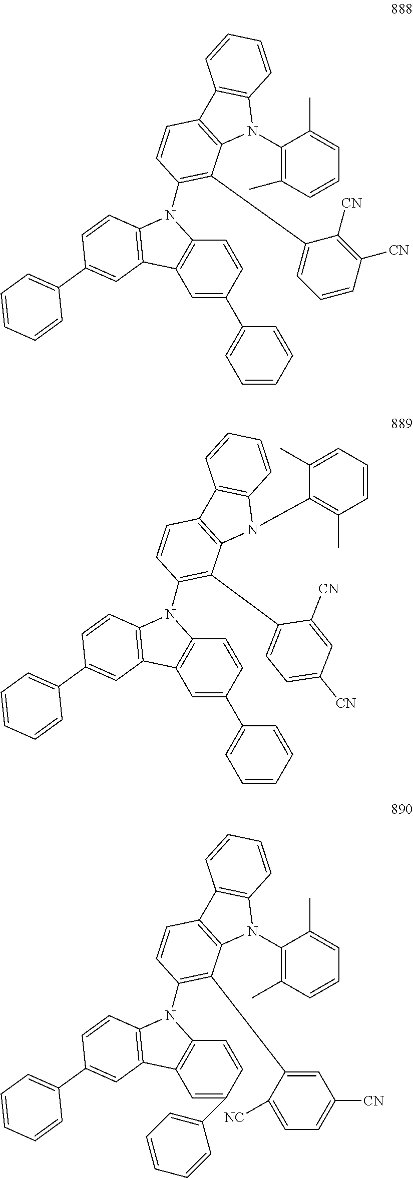 [SMILES: Cc1cccc(C)c1-n1c2ccccc2c2ccc(-n3c4ccc(-c5ccccc5)cc4c4cc(-c5ccccc5)ccc43)c(-c3cccc(C#N)c3C#N)c21 ; Cc1cccc(C)c1-n1c2ccccc2c2ccc(-n3c4ccc(-c5ccccc5)cc4c4cc(-c5ccccc5)ccc43)c(-c3ccc(C#N)cc3C#N)c21 ; Cc1cccc(C)c1-n1c2ccccc2c2ccc(-n3c4ccc(-c5ccccc5)cc4c4cc(-c5ccccc5)ccc43)c(-c3cc(C#N)ccc3C#N)c21] 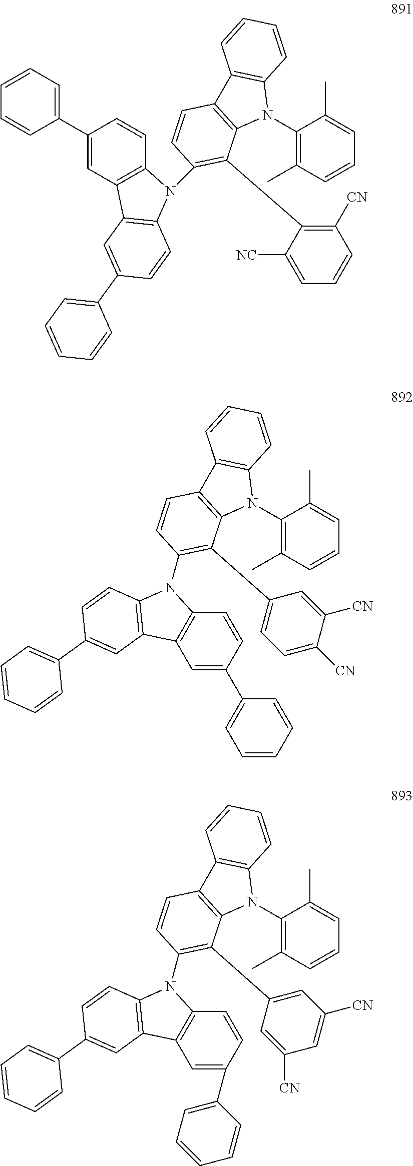 [SMILES: Cc1cccc(C)c1-n1c2ccccc2c2ccc(-n3c4ccc(-c5ccccc5)cc4c4cc(-c5ccccc5)ccc43)c(-c3c(C#N)cccc3C#N)c21 ; Cc1cccc(C)c1-n1c2ccccc2c2ccc(-n3c4ccc(-c5ccccc5)cc4c4cc(-c5ccccc5)ccc43)c(-c3ccc(C#N)c(C#N)c3)c21 ; Cc1cccc(C)c1-n1c2ccccc2c2ccc(-n3c4ccc(-c5ccccc5)cc4c4cc(-c5ccccc5)ccc43)c(-c3cc(C#N)cc(C#N)c3)c21] 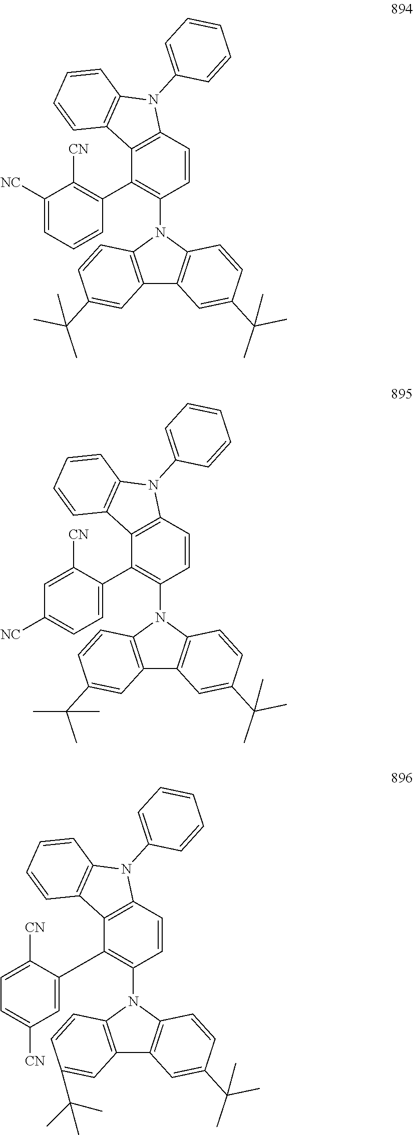 [SMILES: CC(C)(C)c1ccc2c(c1)c1cc(C(C)(C)C)ccc1n2-c1ccc2c(c1-c1cccc(C#N)c1C#N)c1ccccc1n2-c1ccccc1 ; CC(C)(C)c1ccc2c(c1)c1cc(C(C)(C)C)ccc1n2-c1ccc2c(c1-c1ccc(C#N)cc1C#N)c1ccccc1n2-c1ccccc1 ; CC(C)(C)c1ccc2c(c1)c1cc(C(C)(C)C)ccc1n2-c1ccc2c(c1-c1cc(C#N)ccc1C#N)c1ccccc1n2-c1ccccc1] 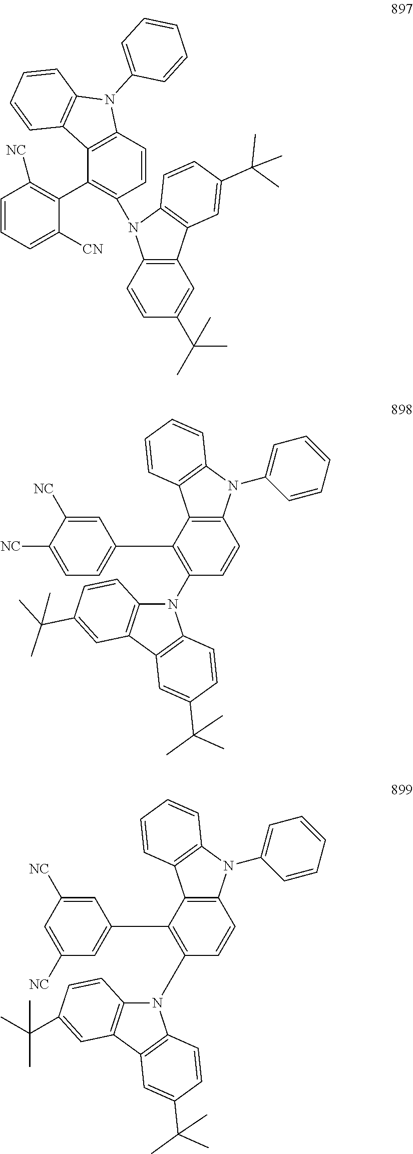 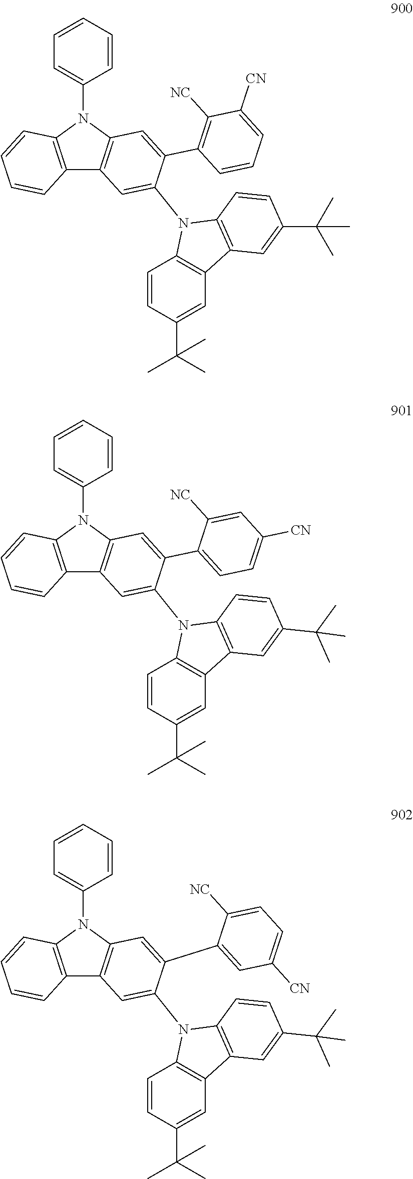 [SMILES: CC(C)(C)c1ccc2c(c1)c1cc(C(C)(C)C)ccc1n2-c1cc2c3ccccc3n(-c3ccccc3)c2cc1-c1cccc(C#N)c1C#N ; CC(C)(C)c1ccc2c(c1)c1cc(C(C)(C)C)ccc1n2-c1cc2c3ccccc3n(-c3ccccc3)c2cc1-c1ccc(C#N)cc1C#N ; CC(C)(C)c1ccc2c(c1)c1cc(C(C)(C)C)ccc1n2-c1cc2c3ccccc3n(-c3ccccc3)c2cc1-c1cc(C#N)ccc1C#N] 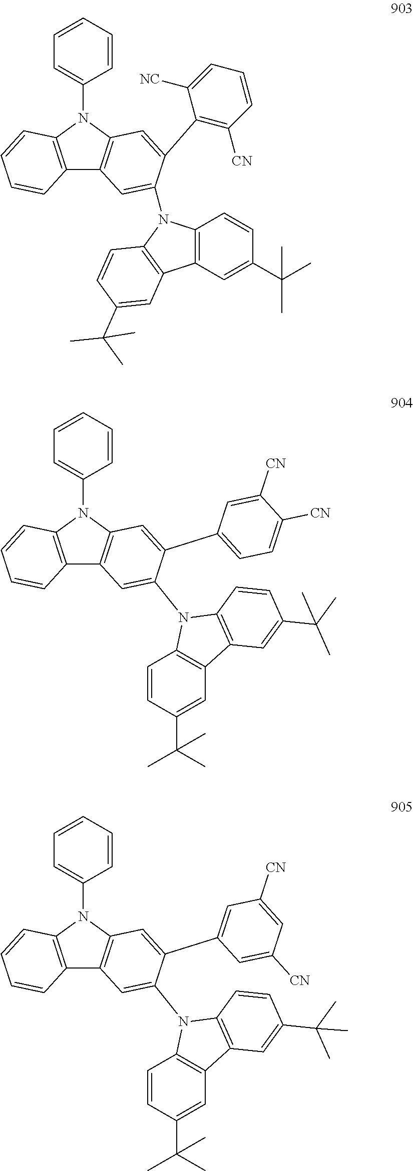 [SMILES: CC(C)(C)c1ccc2c(c1)c1cc(C(C)(C)C)ccc1n2-c1cc2c3ccccc3n(-c3ccccc3)c2cc1-c1c(C#N)cccc1C#N ; CC(C)(C)c1ccc2c(c1)c1cc(C(C)(C)C)ccc1n2-c1cc2c3ccccc3n(-c3ccccc3)c2cc1-c1ccc(C#N)c(C#N)c1 ; CC(C)(C)c1ccc2c(c1)c1cc(C(C)(C)C)ccc1n2-c1cc2c3ccccc3n(-c3ccccc3)c2cc1-c1cc(C#N)cc(C#N)c1] 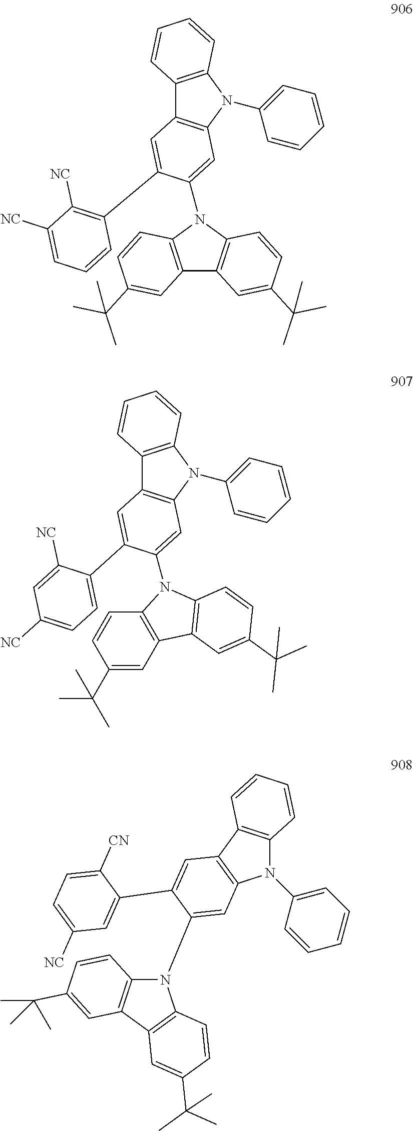 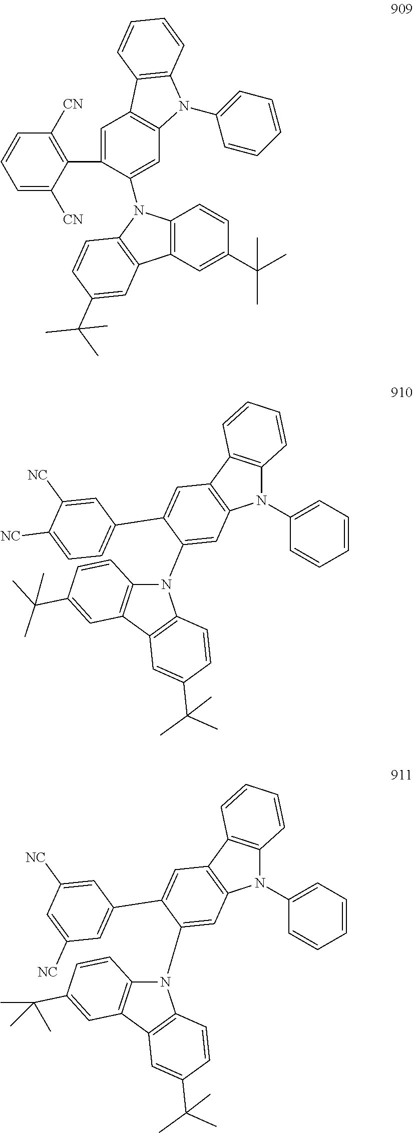 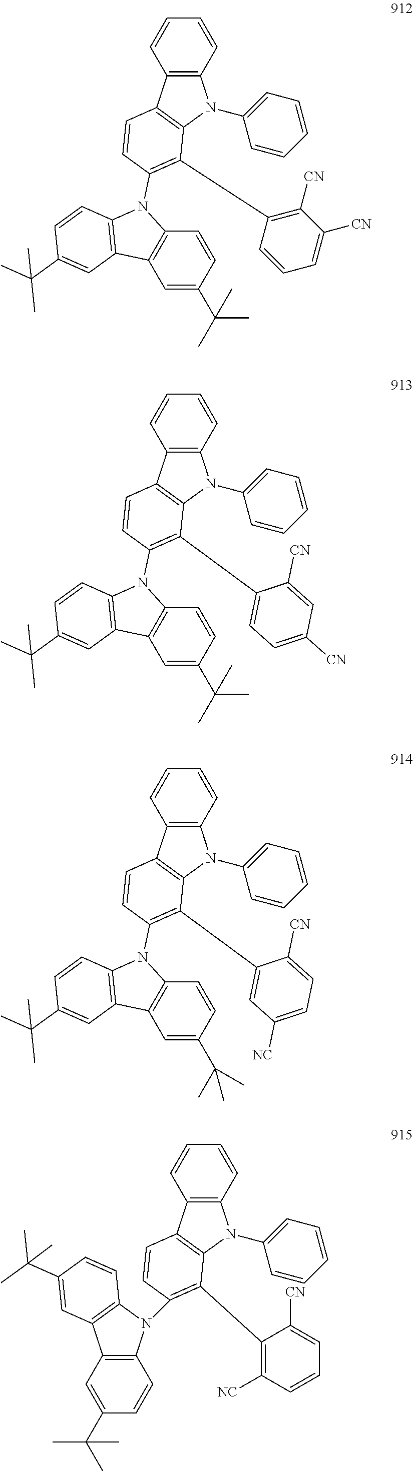 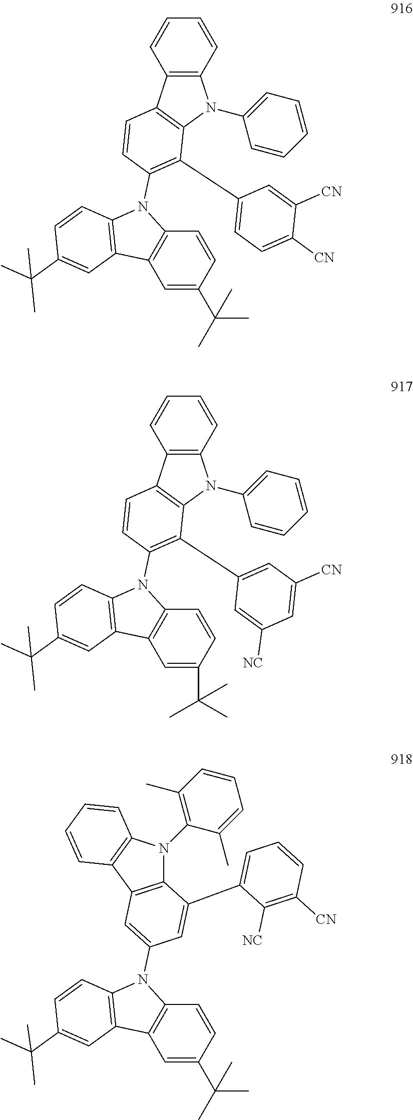 [SMILES: CC(C)(C)c1ccc2c(c1)c1cc(C(C)(C)C)ccc1n2-c1ccc2c3ccccc3n(-c3ccccc3)c2c1-c1ccc(C#N)c(C#N)c1 ; CC(C)(C)c1ccc2c(c1)c1cc(C(C)(C)C)ccc1n2-c1ccc2c3ccccc3n(-c3ccccc3)c2c1-c1cc(C#N)cc(C#N)c1 ; Cc1cccc(C)c1-n1c2ccccc2c2cc(-n3c4ccc(C(C)(C)C)cc4c4cc(C(C)(C)C)ccc43)cc(-c3cccc(C#N)c3C#N)c21] 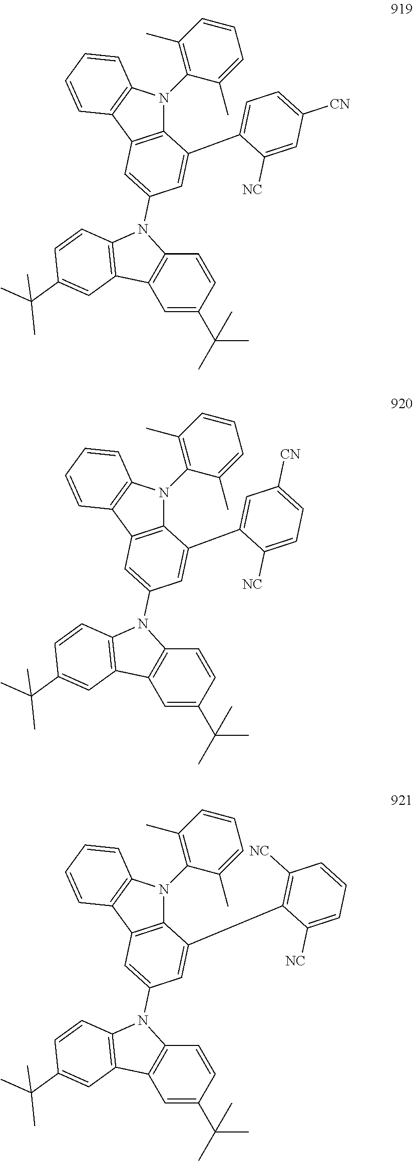 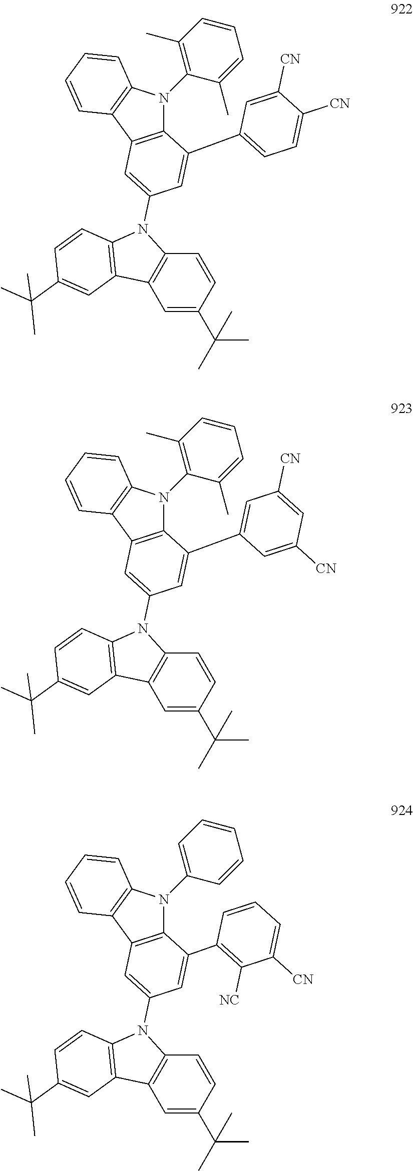 [SMILES: Cc1cccc(C)c1-n1c2ccccc2c2cc(-n3c4ccc(C(C)(C)C)cc4c4cc(C(C)(C)C)ccc43)cc(-c3ccc(C#N)c(C#N)c3)c21 ; Cc1cccc(C)c1-n1c2ccccc2c2cc(-n3c4ccc(C(C)(C)C)cc4c4cc(C(C)(C)C)ccc43)cc(-c3cc(C#N)cc(C#N)c3)c21 ; CC(C)(C)c1ccc2c(c1)c1cc(C(C)(C)C)ccc1n2-c1cc(-c2cccc(C#N)c2C#N)c2c(c1)c1ccccc1n2-c1ccccc1] 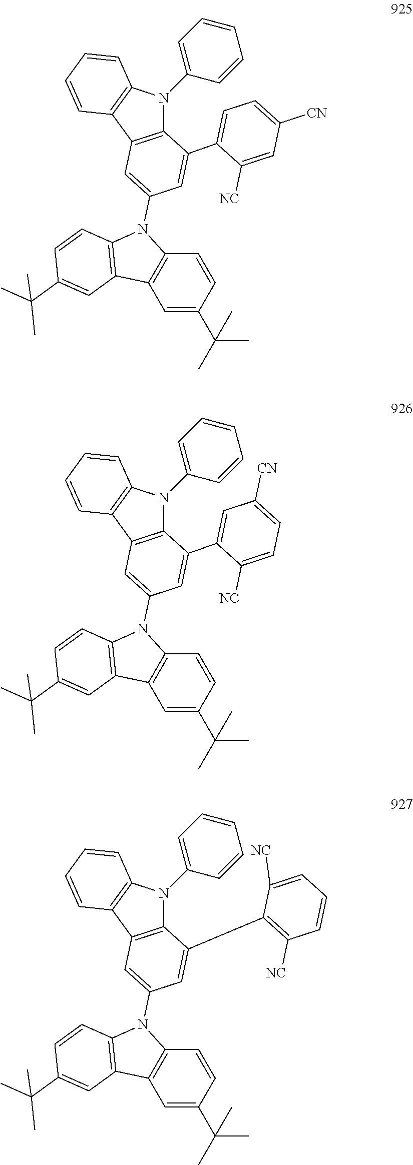 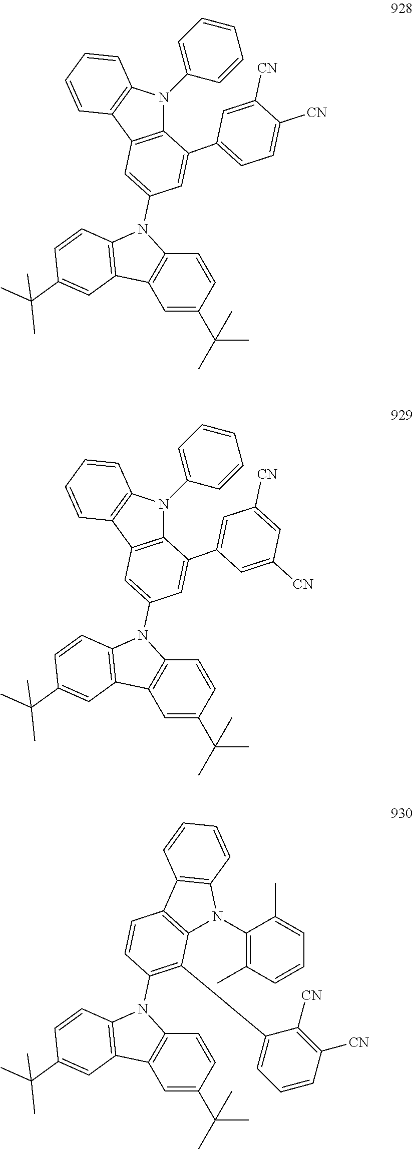 [SMILES: CC(C)(C)c1ccc2c(c1)c1cc(C(C)(C)C)ccc1n2-c1cc(-c2ccc(C#N)c(C#N)c2)c2c(c1)c1ccccc1n2-c1ccccc1 ; CC(C)(C)c1ccc2c(c1)c1cc(C(C)(C)C)ccc1n2-c1cc(-c2cc(C#N)cc(C#N)c2)c2c(c1)c1ccccc1n2-c1ccccc1 ; Cc1cccc(C)c1-n1c2ccccc2c2ccc(-n3c4ccc(C(C)(C)C)cc4c4cc(C(C)(C)C)ccc43)c(-c3cccc(C#N)c3C#N)c21] 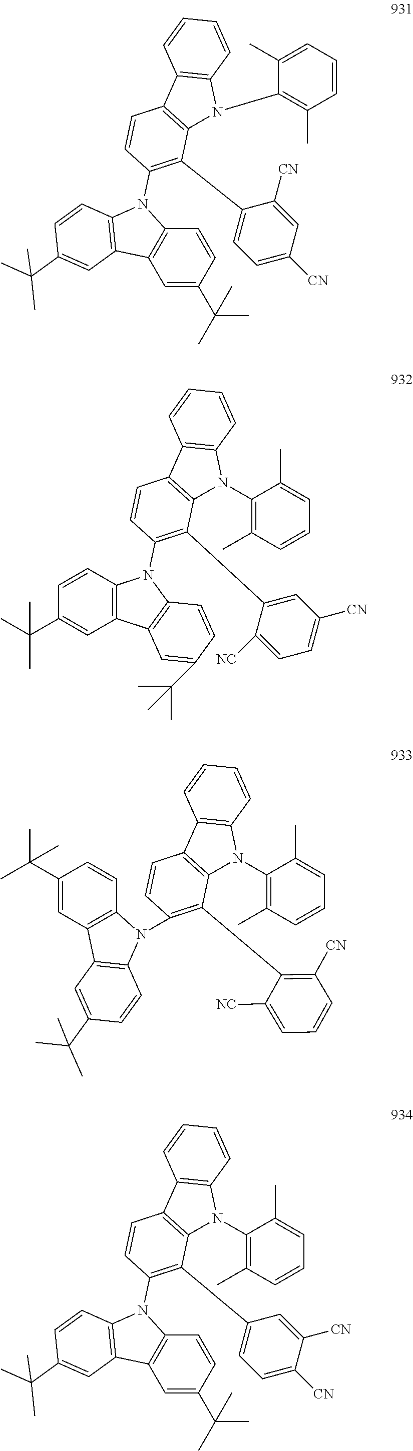 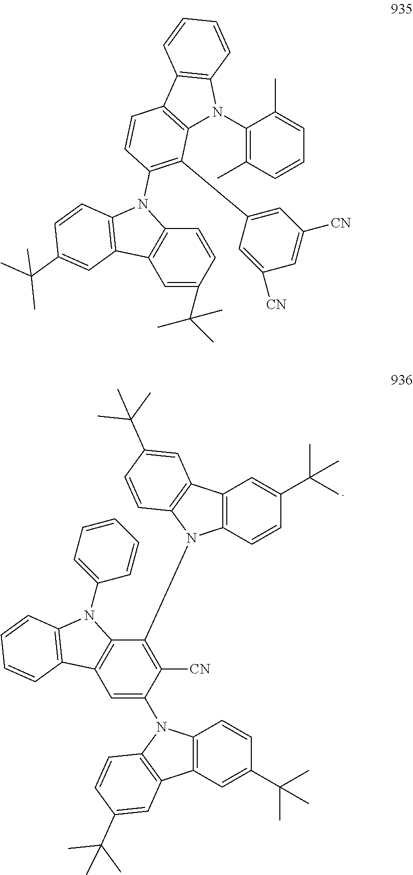 [SMILES: Cc1cccc(C)c1-n1c2ccccc2c2ccc(-n3c4ccc(C(C)(C)C)cc4c4cc(C(C)(C)C)ccc43)c(-c3cc(C#N)cc(C#N)c3)c21 ; CC(C)(C)c1ccc2c(c1)c1cc(C(C)(C)C)ccc1n2-c1cc2c3ccccc3n(-c3ccccc3)c2c(-n2c3ccc(C(C)(C)C)cc3c3cc(C(C)(C)C)ccc32)c1C#N]
